SEQUENCE LISTING (108 sequences)

SEQ ID NO: 1 - protein, 595 aa, Medicago truncatula
MSAFFLPSSSHALFLVLMLFFLTNISAQPLYISETNFTCPVDSPPSCETYVAYRAQSPNFLSLSNISDIFNLSPLRIAKASNIEAEDKKLIPDQLLLVPVTCGCTKNHSFANITYSIKQGDNFFILSITSYQNLTNYLEFKNFNPNLSPTLLPLDTKVSVPLFCKCPSKNQLNKGIKYLITYVWQDNDNVTLVSSKFGASQVEMLAENNHNFTASTNRSVLIPVTSLPKLDQPSSNGRKSSSQNLALIIGISLGSAFFILVLTLSLVYVYCLKMKRLNRSTSSSETADKLLSGVSGYVSKPTMYEIDAIMEGTTNLSDNCKIGESVYKANIDGRVLAVKKIKKDASEELKILQKVNHGNLVKLMGVSSDNDGNCFLVYEYAENGSLEEWLFSESSKTSNSVVSLTWSQRITIAMDVAIGLQYMHEHTYPRIIHRDITTSNILLGSNFKAKIANFGMARTSTNSMMPKIDVFAFGVVLIELLTGKKAMTTKENGEVVILWKDFWKIFDLEGNREERLRKWMDPKLESFYPIDNALSLASLAVNCTADKSLSRPTIAEIVLCLSLLNQPSSEPMLERSLTSGLDAEATHVVTSVIAR

SEQ ID NO: 2 - protein, 595 aa, Lotus japonicus
MAVFFLTSGSLSLFLALTLLFTNIAARSEKISGPDFSCPVDSPPSCETYVTYTAQSPNLLSLTNISDIFDISPLSIARASNIDAGKDKLVPGQVLLVPVTCGCAGNHSSANTSYQIQLGDSYDFVATTLYENLTNWNIVQASNPGVNPYLLPERVKVVFPLFCRCPSKNQLNKGIQYLITYVWKPNDNVSLVSAKFGASPADILTENRYGQDFTAATNLPILIPVTQLPELTQPSSNGRKSSIHLLVILGITLGCTLLTAVLTGTLVYVYCRRKKALNRTASSAETADKLLSGVSGYVSKPNVYEIDEIMEATKDFSDECKVGESVYKANIEGRVVAVKKIKEGGANEELKILQKVNHGNLVKLMGVSSGYDGNCFLVYEYAENGSLAEWLFSKSSGTPNSLTWSQRISIAVDVAVGLQYMHEHTYPRIIHRDITTSNILLDSNFKAKIANFAMARTSTNPMMPKIDVFAFGVLLIELLTGRKAMTTKENGEVVMLWKDMWEIFDIEENREERIRKWMDPNLESFYHIDNALSLASLAVNCTADKSLSRPSMAEIVLSLSFLTQQSSNPTLERSLTSSGLDVEDDAHIVTSITAR

SEQ ID NO: 3 - protein, 594 aa, Pisum sativum
MAIFFLPSSSHALFLALMFFVTNISAQPLQLSGTNFSCPVDSPPSCETYVTYFARSPNFLSLTNISDIFDMSPLSIAKASNIEDEDKKLVEGQVLLIPVTCGCTRNRYFANFTYTIKLGDNYFIVSTTSYQNLTNYVEMENFNPNLSPNLLPPEIKVVVPLFCKCPSKNQLSKGIKHLITYVWQANDNVTRVSSKFGASQVDMFTENNQNFTASTNVPILIPVTKLPVIDQPSSNGRKNSTQKPAFIIGISLGCAFFVVVLTLSLVYVYCLKMKRLNRSTSLAETADKLLSGVSGYVSKPTMYEMDAIMEATMNLSENCKIGESVYKANIDGRVLAVKKIKKDASEELKILQKVNHGNLVKLMGVSSDNDGNCFLVYEYAENGSLDEWLFSESSKTSNSVVSLTWSQRITVAVDVAVGLQYMHEHTYPRIIHRDITTSNILLDSNFKAKIANFSMARTSTNSMMPKIDVFAFGVVLIELLTGKKAITTMENGEVVILWKDFWKIFDLEGNREESLRKWMDPKLENFYPIDNALSLASLAVNCTADKSLSRPSIAEIVLCLSLLNQSSSEPMLERSLTSGLDVEATHVVTSIVAR

SEQ ID NO: 4 - protein, 598 aa, Glycine max
MAVFFPFLPLHSQILCLVIMLFSTNIVAQSQQDNRTNFSCPSDSPPSCETYVTYIAQSPNFLSLTNISNIFDTSPLSIARASNLEPMDDKLVKDQVLLVPVTCGCTGNRSFANISYEINQGDSFYFVATTSYENLTNWRAVMDLNPVLSPNKLPIGIQVVFPLFCKCPSKNQLDKEIKYLITYVWKPGDNVSLVSDKFGASPEDIMSENNYGQNFTAANNLPVLIPVTRLPVLARSPSDGRKGGIRLPVIIGISLGCTLLVLVLAVLLVYVYCLKMKTLNRSASSAETADKLLSGVSGYVSKPTMYETDAIMEATMNLSEQCKIGESVYKANIEGKVLAVKRFKEDVTEELKILQKVNHGNLVKLMGVSSDNDGNCFVVYEYAENGSLDEWLFSKSCSDTSNSRASLTWCQRISMAVDVAMGLQYMHEHAYPRIVHRDITSSNILLDSNFKAKIANFSMARTFTNPMMPKIDVFAFGVVLIELLTGRKAMTTKENGEVVMLWKDIWKIFDQEENREERLKKWMDPKLESYYPIDYALSLASLAVNCTADKSLSRPTIAEIVLSLSLLTQPSPATLERSLTSSGLDVEATQIVTSIAAR

SEQ ID NO: 5 - protein, 557 aa, Cicer arietinum
MSVFFLPSRSHVLFLALMLFLTNISAQSQHLSGTNFSCPVDSPPSCETYVTYIAQSPNFLSLTNISDLFDISPLSIARASNIDDEDKELIPGQVLLVPVTCGCTKHRSFANNTYTIKLGDSYILVSTTSYQNLTNYLEMEDSNPGLNPNLIPPFIKVVVPIFCRCPSKTQLNKGIKYLITYVWHANDNVSTVSSKFGASQVDILTENNYNQNFASAANLPVLIPVTRLPILAQPSSNGRKRSIQLPVIIDKLLSGVSGYVSKPTMYEMDVIMEATMNLSDQCKIGESVYKANIDGKVLAVKKTKKDASEELKILQKVNHGNLVKLMGVSSDNEGNCFLVYEYAENGSLDEWLFLESSKTSDSTVSLTWSQRIGIAVDVAVGLQYMHEHTYPRIIHRDITTSNILLDANFKAKIANFSMARTSTNPMMPKIDVFAFGVVLIELLTGKKGVTTKENGEVVIMWKDFWMIFDLEGNKEERLRKWMDPKLENFYPIDNALSLASLAVNCTADKSLSRPTIEEIVLCLNLLNQPSSEPTLERSLTFGLDVEDTQIVTSIAAR

SEQ ID NO: 6 - DNA, 2144 bp, Cicer arietinum
  source 1..2144: mol_type = unassigned DNA
ttgaaccttcgagctcttgcatataacttattcaatgtcactagccaaattactactgagatttaaacataagtaatctatttttttatatgacaggactttttgatagaggaagaaaatgattggtatgcgaaaagatattttattgacaacaaatgctccatatcaccaatgctaagttcctgcattaagaaacactcttctctcttcccctcataataacttccatttcttcacaactttcacaaaatgtctgtcttctttcttccctctagatctcatgttctttttcttgcactcatgttgtttctcactaacatctcagctcaatcacaacacctcagtggaacaaacttttcatgccctgtggattcacctccttcatgtgaaacttatgtgacatacattgctcagtctccaaattttttaagcctaacaaacatatctgatttatttgatatcagtcctttatccatcgcaagagcgagtaacatagacgacgaggataaagagctgataccaggtcaagtcttattagtacctgtaacttgtggctgcactaaacatcgctctttcgccaataacacctacacgatcaagctcggcgacagctacattctagtttcaaccacttcatatcagaatctcaccaattatcttgaaatggaagattccaaccctggtctaaatcctaatcttattccaccattcatcaaagttgtagtcccaatattctgcaggtgcccttcaaagactcagctgaacaaaggaataaagtatctgataacttacgtgtggcacgctaacgacaatgtttcaactgtaagttccaaatttggtgcatcacaagttgatatattgactgaaaacaattacaatcaaaactttgcttctgcagccaaccttccagttttgattcctgtgacaaggttacctattctagctcaaccgtcttcgaatggaagaaagagaagcattcaacttcctgttataattggtattagtataggaagtgcttttttcgttacagttttaacagtatcacttgtgtatttatactgtctgaaaatgaagagattgaataggactgcttctttatctgagactgcagataagttactttcaggagtttcgggttacgtaagcaagccaacaatgtatgaaatggatgtgatcatggaagctacaatgaacctgagtgaccaatgtaagattggtgaatcagtttataaggctaatatagatggtaaagttttagcagtgaaaaaaactaagaaagatgcttctgaggagctgaaaattctgcagaaggtaaatcatggaaatctggtgaaactaatgggtgtgtcttcggacaacgagggtaactgttttctggtttatgagtatgctgaaaatggttctcttgatgaatggttgttcttggaatcttcgaaaacttcggattcgacagtctcccttacatggtctcagagaataggcatagcagtggatgttgcagttggtctgcaatacatgcatgaacatacttatccaaggataatccacagagacatcacaacaagtaatatccttctcgacgcgaactttaaggccaagatagcgaatttttcgatggctagaacttcaaccaacccgatgatgccgaaaatagatgttttcgcttttggggtggttctgatagagttgctaaccggaaaaaaaggcgtaacaacgaaagaaaatggtgaggttgttattatgtggaaggatttttggatgatttttgatctagaagggaataaagaagagaggctaagaaaatggatggatcctaagttagaaaacttttatcctatagataatgctcttagtttggcttctttggcagtgaattgcactgctgataaatcattgtcaagaccaactattgaagaaattgttctttgtcttaaccttctcaatcaaccatcatctgaaccaacattagaaagatctttgacatttgggttagatgttgaagatactcaaattgttacttctatagcagctcgttgatcaagtgaagataatattaattctgttttctttcatattgaagatggtactttgtttacatgataactatatttttatgcgtggaagtatatggttagtttaattaa

SEQ ID NO: 7 - protein, 597 aa, Phaseolus vulgaris
MAVFFVSLTLGAQILYVVLMFFTCIEAQSQQTNGTNFSCPSNSPPSCETYVTYISQSPNFLSLTSVSNIFDTSPLSIARASNLQHEEDKLIPGQVLLIPVTCGCTGNRSFANISYEINQGDSFYFVATTLYQNLTNWHAVMDLNPGLSPFTLPIGIQVVIPLFCKCPSKNQLDRGIKYLITHVWQPNDNVSFVSNKLGASPQDILSENNYGQNFTAASNLPVLIPVTLLPDLIQSPSDGRKHRIGLPVIIGISLGCTLLVVVSAILLVCVCCLKMKSLNRSASSAETADKLLSGVSGYVSKPTMYETGAILEATMNLSEQCKIGESVYKANIEGKVLAVKRFKEDVTEELKILQKVNHGNLVKLMGVSSDNDGNCFVVYEYAENGSLQEWLFAKSCSETLNSRTSLTWCQRISIAVDVSMGLQYMHEHAYPRIVHRDITSSNILLDSNFKAKIANFSMARTFTNPMMSKIDVFAFGVVLIELLTGRKAMTTKENGEVVMLWTDIWKIFDQEENREERLRKWMDPKLDNYYPIDYALSLASLAMNCTADKSLSRPTIAEIVLSLSLLTQPSPATLERSLTSSGLDVEATQIVTSISAR

SEQ ID NO: 8 - DNA, 2192 bp, Phaseolus vulgaris
  source 1..2192: mol_type = unassigned DNA
ggaaatttagttaaagctaatgacacaaacaggaccatatttttatattaagccaaaagatatttttattgacaaagaactacatatcaacaacgacgattgccagtgatagtagactgcctcataactttcatttgttcacaacttcacatcaatggctgtcttctttgtttctcttactcttggtgctcagattctttatgtggtactcatgtttttcacttgtattgaagctcaatcacaacagaccaatggaacaaacttttcatgcccttccaattcacctccttcatgtgaaacctatgtgacatacatatcccagtcgccaaattttttgagtctgaccagcgtatctaatatatttgacacgagtcctttgtcaattgccagagccagcaacttacagcatgaggaagacaagttgattccaggccaagtcttactgataccagtaacctgtggttgcactggaaaccgctctttcgccaacatctcctatgagatcaaccaaggtgatagcttctactttgttgcgaccactttataccagaatctcacaaattggcatgcagtgatggatttaaacccaggtctaagtccatttactttgccaataggcatccaagttgtaattcctttattctgcaagtgtccttcaaagaaccagctggatagagggataaagtacctgatcactcacgtctggcagcccaatgacaatgtttcctttgtaagtaacaagttaggtgcatcaccacaggacatattgagtgaaaacaactatggtcaaaacttcactgccgcaagcaaccttccagttttgatcccagttacactcttgccagatcttattcaatctccttcagatggaagaaaacacagaattggtcttccagttataattggtatcagcctgggatgcacactactggttgtggtttcagcaatattactggtgtgtgtatgttgtctgaaaatgaagagtttgaataggagtgcttcatcagctgaaactgcagataaactactttctggagtttcaggctatgtaagtaagcctacaatgtatgaaactggtgcaatattggaagctactatgaacctcagtgagcagtgcaagattggggaatcagtgtacaaggctaacatagagggtaaggttttagcagtaaaaagattcaaggaagatgtcacggaggagctgaaaattctgcagaaggtgaatcatggaaatctggtgaaactaatgggtgtctcatcagataatgatggaaattgttttgtggtttatgaatatgctgaaaatgggtctcttcaagagtggcttttcgccaagtcttgttcagagacattaaactcgaggacctcccttacatggtgccagaggataagcatagcagtggatgtttcaatgggtctgcagtacatgcatgaacatgcttatccaagaatagtccacagggacatcacaagcagtaatatccttcttgactccaactttaaggccaagatagcaaatttctccatggccagaacttttaccaaccccatgatgtcaaaaatagatgtatttgcttttggggtggttctgatagaattgcttactggcaggaaagccatgacaaccaaagaaaatggtgaggtggttatgctgtggacggacatttggaagatctttgatcaagaagagaatagagaggagaggctcagaaaatggatggatcctaagttagataattattatcctattgattatgctctcagcttggcctccttggcaatgaattgcactgcagacaagtctttgtccagaccaaccatagcagaaattgtccttagtctctcccttctcactcaaccatctcccgcgacactggagagatccttgacttcttctggattagatgtagaagctactcaaattgtcacttccatctcagctcgttgattgagtgaagccaatctagtttctcacatccaagatggtacttttttttaaataatgattgcaccttagtcaataatgatgaacttggggagttttcaacatttagtgtttccatccctgttgttctttatgtttgaggtagagttcgtaaaacgaatagcaattgcagttctcctcagactaaatttgcttatttctctgtacttcttttatatgacaattgaaagtgaatcaaatgatggag

SEQ ID NO: 9 - protein, 595 aa, Arachis hypogaea
  source 1..595: note = subsp. Hypogaea
MAFFLPSLSSSIFLAFMLFSVTSIPTQSQQVNGTDFSCPVDSPSSCGTYVTYIAKSPNFLSLSNISDIFDTSPLSIARASNIKNEGDKLVPGQVLLIPVTCGCTQNQSFANITYELRQGDVYDIVSKTTYENLTNWRAVNNSNPDLNPVLLPIGVKVLFPLFCRCPSKKQLQKGIEYMITYVWQNNDNVSSVAAKFGASPVDILSENNYGGNFTAATYLPVLIPVTKLPVLTQPEASHGRKRSIQIPVIISISLGFTLVVAVIVISMVYAYLYQRKRTLNRGDLSAGTADKLLSGVSGYVSKPTVYEANEVIKATMNLSGQCKLGGTVYKAKIEGQVLAVKKVNQVVSEELNILQKVNHGNLVKLMGVSSDSDGNHFLVYEYADNGSLDGWLFSKLSLKASLTWYQRINIALDVAMGLQYLHEHTYPRIVHRDITTSNILLDSNFKAKIGNFSMVRTTTNPMISKIDVFAFGAVLIELLTGMKAMTTKADGEVVMLWKDIRKMFEVEDEKEKEECLRRWMDPKLECLYPVDYALSLATLAANCTADVSLSRPTMAEVVLGLSLLTQPSQAALERSLTSSALEAEVTHVATSITAR

SEQ ID NO: 10 - DNA, 2683 bp, Arachis hypogaea
  source 1..2683: mol_type = unassigned DNA; sub_species = Hypogaea
atctttctcttaacagtagtagctagattacagaatctcttattctattgttttcgattagattagatggaatcacgtatatgaaccgacaccgtgaagcatgaaagccgcactcaaatattcaagaaggctatttcaaaaatatgaaaccagggaaaaaatgcaagtgtaaaataatgcttatgaaaaattgtatgcagtgtaggtagtaattcttataaaattcattagacccgaatacttttagcacgtataaatcttgtggagacactacattttaaggcgtggtctagtgatagatagacatggaattggcatcagaattttctcttatgatgtctcaaattggagattctttgcatataagttaaaaatgaccatcctggaatcttcattggcattttattatactctgctgtgcgcatgaatagcatactattgatatatgtaaagtcaatgttttccttaacatataatcttagacttacttggaaatttgatatcactgcataaataattaggaatagatatggtaaaacagtataccaataactcttctgttaaaccaaagggtattttattgacaaagaatcctccatatcaccaaaaatcctgagtgtctttgacttctgataataaaagtttcctttatgttctttccctccttctcaacttcagaaaaatggctttctttctaccctctctctcaagtagtatttttcttgtattcatgttctccatcaccagcatcccaactcaatcacaacaggttaatggaacagacttttcatgcccagtggattcaccttcttcctgtggaacatatgtgacatacatcgctaaatctccaaacttcttgagcctttctaacatatctgacatatttgacaccagccctttatccattgcaagagcaagtaacataaagaatgagggtgacaagctggttccaggccaagtcttactgatacctgtcacttgtggttgcactcaaaaccaatctttcgccaatatcacctatgagctaaggcagggtgatatgtacgactttgtctcaaaaacaacatatgagaatctcacaaattggcgtgctgtcaacgattcaaacccagatttgaatccagttctgctgccagtaggtgtgaaagtattgttccctttattctgcaggtgcccttctaagaagcagttacaaaaagggatagaatatatgatcacctatgtgtggcagaacaatgacaatgtttcctctgtagcagccaagtttggtgcatcggcagtggacatattgtccgaaaacaactatggtggaaacttcacagctgcaacctatcttccggttttgattcctgtgacgaagttgccggttcttactcaacccgagccttcacatggaagaaagagaagcattcaaatccctgttataatcagtattagcctggggttcacccttgttgttgctgttatagtaatatcaatggtttatgcttatctttatcagagaaagaggactttgaataggagagactcatctgctgggacagcagataagctactctctggagtctcaggctacgtgagtaagccaaccgtgtatgaagccaatgaggttatcaaagccaccatgaatctcagcgaacagtgcaagcttgggggcacagtttacaaggccaaaatagaagggcaggtcttggcagtgaaaaaagtgaatcaagtagtttctgaggagctgaatattctgcagaaggtgaatcatggaaacctggtgaaactgatgggtgtatcttcagacagtgatggaaaccatttcctggtttatgagtatgctgataacgggtcccttgatgggtggctcttctccaagttgtctttgaaggcctcgcttacatggtatcagaggattaacatagcattggatgttgccatgggtctgcaatacttgcatgagcacacttatccaagaatagtccatagggacatcacaacaagtaacatccttcttgactccaacttcaaggccaagatagggaacttctccatggtcagaactactacaaatcccatgatttccaagatcgatgtctttgctttcggggttgttctgattgagttgcttacaggcaggaaagccatgacaacaaaggcagatggtgaggtagtaatgctgtggaaggatattaggaagatgtttgaagtggaagatgaaaaggaaaaggaggaatgtctgagaagatggatggatcctaagctagagtgcctttaccctgtggattatgctctcagcttggccacgttggccgcgaattgcacggcggatgtatcattgtctagaccaaccatggcagaagttgttcttggcctctcccttctcactcaaccatctcaagctgcactagagagatcattgacttcttctgcgttggaagcagaggttactcatgtggctactcccatagcagcacgttaattggtgacgaaagtaattcagtttctcaggttcaagagagtgttttcttcacatgactactgcctataagctttgttaataagtgtccaagtttattgtcgctttaaagtttctttgtttcatcccttatattctttatctgtttgtagtcgaggaatgactgcttatttctctcaagttgacatatatgatatatataacaaaaagaatattttg

SEQ ID NO: 11 - protein, 591 aa, Lotus japonicus
MTSFFLFTNTLFLALMMFFSTTHHILAQLSHTNGTNFSCPVDSPPSCDTYVTYFAQSPNFLTLTSISDLFDTSPLSIARASNIKDENQNLVPGQLLLVPVTCACSGSNSFSNISHMIKEGESYYYLSTTSYENLTNWETVQDSNPNYNPYLLPVGIKVVIPLFCKCPSNYHLNKGIEYLITYVWHNNDNVSLVASKFGVSTQDIISENNFSHQNFTAATNFPILIPVTQLPSLSQSYSSSERKRSNHIHIIISIGISLGSTLLIALLVLVSVTCLRKRKSSENKSLLSVEIAGKKLISGVSNYVSKSILYEFRLIMEATLNLNEQCKIGESVYKAKLDGQVLAVKKVKEDVTEEVMILQKVNHLNLVKLMGVSSGHDGNHFLVYEFAENGSLHNWLFSNSSTGSRFLTWSQRISIAVDVAMGLQYMHEHTQPSIVHRDITSSNILLDSNFKAKIANFSVARTSINPMILKVDVFGYGVVLLELLSGKKSLTNNEINHIREIFDLKEKREERIRRWMDPKIESLYPIDDALSLAFLAMNCTSEKPLSRPTMGEVVLSLSLLMTQHSPTTLERSWTCGLDVDVTEMQTLIAAR

SEQ ID NO: 12 - protein, 590 aa, Medicago truncatula
MVSSFFHTLIFFSATHILLQLPQANGKNFSCTLNSSPSCDTYVAYFANSPNFLTLTAISDIFDTSPQSIARASNIKDENMNLIHGQLLLIPITCGCNGNGNYSFANISHLIKESESYYYLSTISYQNLTNWQTVEDSNPNLNPYLLKIGTKINIPLFCRCPSNYFAKGIEYLITYVWQPNDNLTLVASKLGASPKDIITANTNNFGQNFTVAINLPVFIPVKNLPALSQSYYSSSERKRINHFSIIISIGICLGCTILISLLLLLFYVYCLRKRKACENKCVPSVEITDKLISEVSNYVSKPTVYEVGMIMKATMNLNEMCKIGKSVYKAKIDGLVLAVKNVKGHITVTEELMILQKVNHANLVKLVGVSSGYDGNHFLVYEYAENGSLYNWLLSEFCTLSWSQRLSIAVDIAIGLQYLHEHTQPCIVHRNIKSSNILLDSKFKAKIANFSVARTTKNPMITKVDVLGYGMVLMELITGKKFLSYSEHSEVNMLWKDFKCVFDTEQKREEIVRRWMDPKLGRFYNVVEALSLFTLAVNCIEEQPLLRPTMGEVVLSLSLLTQPSPTLLEVSWTYGLDVEVAEMVTPIIAR

SEQ ID NO: 13 - protein, 613 aa, Parasponia andersonii
MAISLYLLLFFITHISAQSPPTLATNFSCSTNSSQPSCKTYVAYFAQPPLFMDLKSISNLFGVSPSSISEASNLVSESTKLTRGQLLLIPLSCSCNGSHYFSNVTYNITMGDSYYLVSIHSFENLTNWPLVRDTNPTLNPNLLQIGTKVIFPLYCGCPSKSHSKNGIKYLITYVWQPSDDIYRVSAMFNASEVDIIIENNYQDFKAAVGYPVLIPVSRMPALSQPPYPSHSHHRSQLKHRWFLIAVISSAGALLILFLATFLVHSIGLYEKKKNLSHEESSLETTDLIQVKNFSKSDTLELQAKHDKLLPGVSVYLGKPIMYEIKMIMEATMNFNDQYKIGGSVYRAMINGSFLAVKKAKENVTEELHILQKVNHGNLVKLMGISLDRDGNCFFVYEYAENGSLDKWLNPQSSTSTSSSVGILSWSQRLNIALDVANGLQYMHEHTQPSIVHKEIRTSNILLDSRFKAKIANFSMARSAASAGMTKVDVFAFGVVLLKLLSGRKAMATRENGEIVMLWKEAKAVLEEEEKRAEKVREWIDPKLESFYPIDGALSLMTLAKACTQEKASARPSIGEVVFSLCVLTQSFSETLEPSWTCTLEGEDVVQITSPIVA

SEQ ID NO: 14 - protein, 582 aa, Parasponia andersonii
MADSYFPFQAIFLLLLLFSTLNMAASQLNNSATDFSCSDSPPSCEAYVAYFSQPPNYMNVGNISDLFGISQALIAKSSNLVSKDSPLIPQQLLLIPLTCTCTGNHYFANITYQVEPGDTYHYLSTLLFENLTNSQVMKKMNPEISPEYVLPYIDIIIPVFCRCPSKSHLKSEIQQFITYVWQPNDQVSNVSAKFNTSASEIVNENKYNNFSSAVGLPVLIPVSKLPVLARVKPPKSVRSKKQWILIGVESLGGIVLITLFATLLVYSNRLLKKRRKILEARRLEPRIIIQDKLLSGVSEYLGRPIMYDNKMVVEGTMDFSEQCRIGGSVYRGEIYGEVFAVKKTKQDITDELNLLQKVNHVNLVNLMGASYDTDGNRFLVYEYVENGSLERWLDLKPSSLAAASSSSSVQFLSWSQRIQIALDVANGLQYLHEHTQPNIAHWNIRTSTILLDSKFRAKIANFEVARPVGNPAMLKVDIFAFGIVLLALVSGKKALQTIENGEVIMLWKDLAKEVFEVEEKKEDRLRKWMDPNLQSFYPIDGALSLSSLARACIREKSSARPKMAEIVFSLSVLAQSSSPGTP

SEQ ID NO: 15 - skipped

SEQ ID NO: 16 - protein, 670 aa, Hordeum vulgare
MAAPPGRRGLAFGTAALALLAILAVARGQQQYEANAQTNCYGRNGSSVLGYVCNATAAAAPCATYVVFRSSPPYYGTAVSISYLLGSDPEAVADANGVPTVSPLADSRLVLAPVPCGCSPRGYYQHNSSHTIELRGETYFIIANNTYQGLTTCQALLAQNPRHGSRDLVAGNNLTVPIRCACPTPAQAAAGVRHLLTYLVTWGDSVSAIADRFRVDAQAVFQANNLTAREIIFPFTTLLIPLKSAPTPDMLVSPAPPPAPAPPQAQQPPASGSGKWIAVGVGVGVGVLALASLIGLMLLCVRRRRTRQGVRERGRLSKVVLDVPSSADYNALASGKHASSATTTSASSSALVSSDARAAVESLTVYKYSELEKATAGFSEDRRVKNASVYRAEINGDAAAVKRVAGDVSGEVGILKRVNHSSLVRLSGLCVHHGETYLVFEFAENGALSDWLHGGGATLVWKQRVQAAFDVADGLNYLHHYTNPPCVHKNLKSSNVLLDANLRAKVSSFALARSVPTGADGGDAQLTRHVVGTQGYLAPEYLEHGLITPKLDVFAFGVILLELLSGKEAMFNGGDKRGETLLWESAEGLVVDNEDARGKVRPFMDPRLHGDYPLDLAVAVASLAVRCVAREPRRRPSIDVVFATLSAVYNSTLDWDPSDDGNSRSSIVGR

SEQ ID NO: 17 - protein, 650 aa, Hordeum vulgare
MAPLTRRRRLLATLLCLCALPAPARSQNASATPAPASVEGFNCSANGTYPCQAYALYRAGLAGVPPDLSAAGDLFGVSRFMLAHANNLSTSAAPAAGQPLLVPLQCGCPSGSPNAYAPTQYQISSGDTFWIVSVTKLQNLTQYQAVERVNPTVVPTKLEVGDMVTFPIFCQCPTAAQNATALVTYVMQQGDTYASIAAAFAVDAQSLVSLNGPEQGTQLFSEILVPLRRQVPKWLPPIVTRNDASATPPSPSPPPTTTPGPSDVADNRDGVVTGLAVGLGVVGGLWLLQLLLLGCLWRRLKAKGRRGDAVASGEGGEGGRSAKTASASGGVGGERFLVTDISEWLDKYRVFKVEELERGTDGFDDAHLIQGSVYKANIGGEVFAVKKMKWDACEELKILQKVNHSNLVKLEGFCINTATGDCFLVYEYVENGSLDLCLLDRGRARRLDWRTRLHIALDLAHGLQYIHEHTWPRVVHKDVKSSNVLLDARMRAKIANFGLAKTGHNAVTTHIVGTQGYIAPEYLVDGLVTTKMDVFAYGVVLLELVSGREAAGDGGDLLLADAEERVFRGREDRLEARAAAWMDPVLAEQTCPPGSVATVMGVARACLQRDPSKRPSMVDVAYTLSRADEYFADYSGESVSVDGSGEIAAR

SEQ ID NO: 18 - protein, 681 aa, Hordeum vulgare
MATPTRWRGLAAVGRAALAFLVLLAVAAPWCPVARGQQEYEANAQNNCYGNNGSSVLGYTCNATAAVRPCASYVVFRSSPPYESPITISYLLNTTPAALADANAVPTVSSVAASRLVLAPLNCGCAPGGYYQHNASYTLQFSNETYFITANITYQGLTTCQALMAQNPNHDSRNLVVGNNLTVPIRCACPSPAQAASGVRHLLTYLVASGDTIADIATRFRVDAQAVLRANRLTDSENIYPFTTLLIPLKSAPTPDMLVSPAPPPAPVPPQAQQPLPTGGSGSGKGVAIGVGVGVGVLALAGLLGLMFLCVRRRRRLRPGVGENGHPGKVVIDVPSSADYDPLASGKHTSSATTTSSSSSAFVSSDARAAVESLTVYKYSELEKATAGFSEDRRVKDASVYRAVINGDTAAVKRVAGDVSGEVGILKRVNHSSLVRLSGLCVHHGDTYLVFEFAENGALSDWLHGGGATLVWKQRVQAAFDVADGLNYLHHYSTPPCVHKNLKSSNVLLDADLRAKVSSFALARSVPTGAEGGDAQLTRHVVGTQGYLAPEYLEHGLITPKLDVFAFGVILLELLSGKEATFNGGDKRGEKLLWESAEGLVVDGEDARSKVRAFMDPQLSGDYPLDLAVAVASLALRCVAREPRGRPSMYEVFVTLSAVYNSTLDWDPSDYSNSRSSIVGR

SEQ ID NO: 19 - protein, 633 aa, Hordeum vulgare
MEPRRFLCCCLVAVLAVASRRCDAQGGAGNGTGRFACVVPAPCDTFVLYRTQSPGSLDLGAISDLFGVSRAMIAAANNLSLIDEDAALLPDQPLLVPVRCGCTGNRSFVNVTYPIHSGDTFYALALTGYENLTTPDVIQELNPQAVFNKLNVSQLVTVPLFCRCPTPAERSAGVLQQITYMWRPVDTMSRVSKLMGSDASAIAAANNVSADFTSTTMLPMLIPVARPPVLPPLRYGPSATTGDPGATKRFSGATVAASIAGSLVAVAALCVAIFGYRRYRRKKATVHSASRFASPRFCFNQNAYGIQSSSSIARMINGGDKLLTSVSQFIDKPVIFGTAEIMEATMNLDERCRIGSSYYRAKLEGEVFAVKPAKGDVSAELRMMQMVNHANLIRLAGISIGADGDYTFLVYEFAEKGSLDKWLYQKPPSSLPSSSSSVDTLSWNQRLGIALDVANGLLYMHEHTQPSMVHGDVRARNILLTADFRARISNFSVATPAMADAAATSSDVFAFGLLVLELLSGRTAMEARVGAEIGMLWRDIRAVLEAGDKRDAKLRKWMDPALGDEYYLDAALSLAGMARACTEEDAARRPKMADVVFSLSMLVQPLPVGDAFEKLWQPSSEENIRIVNEVAAR

SEQ ID NO: 20 - protein, 638 aa, Zea mays
MEPRHFCRALLLLLVVLLLGFRRAGAQDSTNYTVPARFACNVSSPCDTYVVYRTQSPGYLDLGSISDLFGTSQARIASANGLSSEDGVLQPGQPLLVPVRCGCTGAWSFANATYPIRQGDTFYNLARLSYENLTEYHLIHDLNPRSEPTSLQIGQEVTVPLLCRCPPARAVQSFITYVWQPGDTLSQVSKLMNATADEIAEANNVTSSSVSSASAAGLPMLIPVQQRPRLPPLLYAASAGEGRSSRSRRRALIIIGASVSGSLVALAALLVAIMAQRRYRRKKPSMRLGSPFAVNTKLSWSVNQYGHGSSNSFAHVMKGGKLLTGVSQFIDKPIIFVEEEIVEATMNLDERCKIGSTYYRAKLDGEVFAVKPAKGDVSAELRMMQMVNHANLIKLAGISIGADGDYAFLVYEFAEKASLDKWLYHNHQKPPSALLPSSSCTVPTTLSWGQRLSIALDVANGLLYMHEHTQPSMVHGDIRARNILLTADFRAKISSFSLAKPATADAAATSSDVFAFGLLLLELMSGRRAMEARIGSEIGMLWREIRAVLEAGDKREAKLRKWMDPALGSEYQMDAALSLAGMARACTDEDAARRPNMTEVVFSLSMLAQPLSVADGFEKLWQPSSEDNIRIAGSVAAR

SEQ ID NO: 21 - protein, 636 aa, Zea mays
MELRHFRCCASRLLLLVTLLLGFRRAGAQDSTSYTVPAQFACDVSSPCDTYVVYRTQSPGYLDLGSISDLFGTSQARIASANGLSSEDGVLQPGQPLLVPVRCGCAGAWSFANVTYPIRQGDTFYNLAKASYENLTEYHLIQNLNPGSEPTSLQIGQEVTVPLLCRCPARAERSRGVQSLITYMWQAGDTMSQVSKLMNATVDEIAEANNVTANTSASASFVGQPMLIPVRQRPRLPAPLYAAAAADGKSRSRRRAAVIGASVSGSLVALAALFVAILARRRYRKKPSMRLGSRFAVNTKLSWSRNQFGHDGSNSFAHVMKGGKLLTGVSQFIDKPIIFVEEEIMEATMNLDERCKIGSTYYRAKLDGEVFAVKPAKGDVSAELKMMQMVNHANLIKLAGISIGADGDYAFLVYEFAEKGSLDKWLYEKPPSALPSSSCTVATLSWGQRLSIALDVANGLLYMHEHTQPSMVHDDIRARNILLTADFRAKISGFSLAKPAMVDAAATSSDVFAFGLLLLELLSGRRAMEARIGSEIGMLWREIRGVLETGDKREAKLRKWMDPALGSEYHMDVALSLASMARACTEEDAARRPNMTEVVFSLSVLAQPLSVADGFEKLWQPSSEDNIRIAGSVAAR

SEQ ID NO: 22 - protein, 620 aa, Malus domestica
MAISFLCSKPLCILLLLLFFTARILAQSTPSNSSTSFSCSVDAPPSCDTYVSYFARPQFMSLENISHLFGVSPLSIAKASNLVSEHIRLIAGQLLLVPISCGCSGNSYFSNITYEIKSGDSFYLVSINSFENLTDWHEVLNMNPTLDPSLLQIGQKVIFPLFCKCPSKMYTENGIKYHITYIWQPNDDISRVSSRFNVSTLDISSANNLHNDSAAVELPVVIPVSRLPALVQPKPPQGRNIFKQRWWLILIIILGGVLLVSSLLAIFAVYTRHQHKVKKALDGPGSSLESAEWFKMKEGKIDENFDLKFIQDKLLPGVSSYLGKPIMYEVKTIMEATMNLNEHCRIGGSVYRAIVDGQVLAVKNTKEDVTEELNILQKVNHANLVKLMGVSSETDGSRFLVYEYAANGSLDKWLYSKSSATSSSAELLTWNQRLSIALDIANGLQYMHEHTQRSIVHMDIRTSNILLDSKFKAKIANFSMARAAANDVTPKVDVFAFGVVLLALLSGKKGMEAKENGEAIMLWKDVRWVLEAEEEKVERLRKWMDPNLENFYPIDGALSLTALARACTQEKPSTRPSMGEVVFNLSVLTHSSSQSTLERSWTSALEAEEVLETISPIAAR

SEQ ID NO: 23 - protein, 624 aa, Fragaria vesca
  source 1..624: note = subsp. Vesca
MAVSFLCSSMVCILLLFFFTSQILAQPAPQSNSTTSFSCAVDAPSSCETYVAYFVESPGYMNLENISDLFGVSVSSISQASNLASSYTGQTRLVAGQLLLVPITCGCTGNRSFANITYSIKRGDSYYVVSMYTFENLTRWPLVVEMNPALVPSLLQIGVKVIFPLFCKCPSKMYSDLGIKYLLTYVWQTNDDIFRVSAKFNISALNISGANNFDNGSPVVGQPVLIPLTKLPALSQPLPPHGKHIFKHRLMLIVIICLGVALSVASLLAIFLVHTHRLRKRQKLLNDKSLSLESAEWFRMKEGKSEEKIEMKFIQDKLLPGVSSYLGKAILYDVKTIMEATMNLNDHCGIGGSVYRAVIDGKVLAVKKTKEDVTEELNILQKVNHANLVKLMGISSEIDGVRFLVYEYAENGSLDKWLYHKTSTNSSSGAFLTWSQRLSIALDVANGLQYLHEHTQPSIVHMDIRTSNILLDSKYKAKIANFSMARTAANSVTPKVDVFSFGVILLSLLSGKKGMETTDNGEVIMLWKDVRGVLEAEEKKQEKLRAWMDPTLESFYPIDGALSLTALASACTQEKSSARPSMAEVVFNLSVLTHSSSESTLERSWNSALEVEEVLQTISPIKAR

SEQ ID NO: 24 - DNA, 1317 bp, Lotus japonicus
  source 1..1317: mol_type = unassigned DNA
ggacatgagattgaagctccaaaattagctcttttttctgatgaatacttaatgctttgttgtattcacttgattaagtgctagaaatcatctttgcatgatcatagattaaatgaatttccagttggtgtgtggagagctattttgttatgctgacatctgcaatttgcagggcatctaatgattgtcatttcttaaattattattggttgtttccgtttctttaattatctgttttaatcttgcaggtcatacaaattaaaatactagccaccacccaagacatactaaatggggtagtagagggaagggtaaggtcgataaggatgactttttattctataaaatttaggagaatttgagcttaagtggcaaggcaaacgacattactatacgaattggctttgtaccagaaacagggaacaaataatattttacaaataagctattatcatgtcagctcatttgttcaactttgatttgattaaaaattaaatgaagttgaatttgttgagctgctttattatatatgccactggatgtttccgcattctaagtgcatgtttgaaaacatttctacaattgattacgaaggaaaaattaatcatggagagaagcttatgtgcgtagcttctgtatttctgaattgattctatctgtacagtagcatttagataatgaatgatcttggttctcgctaagcatcaaaccaatctctacccttttaaaattgcaagaattataagtcatgcattgacccaaatccttctgtggttatgccccttaaaaatccggcaagacatcaagttagttggtcattagggttccaccagctagctgacaccttgtacaacaactggccgtcctaaagttgggtaagcattacaatactaaatgccattttattatattttgcgcatggttatatacctaagtaggatttgtccacagtttctttgattcggaaaggaaaaaatatttagttgacactgacagaagcagattttatatacatatattatgaaatgactcctacatgagatacacgaatctcatccccatgagttgcagtttgacagagtacacacttatcaacttgctggaatataggaaagtctaaccaatgatgtcgatccgtattgccttaattttggtaaatttagtattacatgatcattattgatatactaaaccacaggatattttattgacaatgtgaatgttccatattttcaacaatgctgattccctctgataaagaacaagttccttttctctttccctgttaactatcatttgttccccacttcacaaaca

SEQ ID NO: 25 - DNA, 1317 bp, Lotus japonicus; identical to SEQ ID NO: 24
  source 1..1317: mol_type = unassigned DNA

SEQ ID NO: 26 - protein, 61 aa, Medicago truncatula
CTKNHSFANITYSIKQGDNFFILSITSYQNLTNYLEFKNFNPNLSPTLLPLDTKVSVPLFC

SEQ ID NO: 27 - protein, 61 aa, Lotus japonicus
CAGNHSSANTSYQIQLGDSYDFVATTLYENLTNWNIVQASNPGVNPYLLPERVKVVFPLFC

SEQ ID NO: 28 - protein, 12 aa, Lotus japonicus
PGVFILQNITTF

SEQ ID NO: 29 - protein, 9 aa, Lotus japonicus
LNDINIQSF

SEQ ID NO: 30 - protein, 11 aa, Lotus japonicus
NGSNLTYISEI

SEQ ID NO: 31 - protein, 9 aa, Lotus japonicus
ASKDSVQAG

SEQ ID NO: 32 - protein, 67 aa, Lotus japonicus
CLKGCDLALASYYILPGVFILQNITTFMQSEIVSSNDAITSYNKDKILNDINIQSFQRLNIPFPCDC

SEQ ID NO: 33 - protein, 68 aa, Medicago truncatula
CVKGCDVALASYYIIPSIQLRNISNFMQSKIVLTNSFDVIMSYNRDVVFDKSGLISYTRINVPFPCEC

SEQ ID NO: 34 - protein, 622 aa, Lotus japonicus
MEHPRLGFPITLLLFSFILLPSTSQSKCTHGCALAQASYYLLNGSNLTYISEIMQSSLLTKPEDIVSYNQDTIASKDSVQAGQRINVPFPCDCIEGEFLGHTFQYDVQKGDRYDTIAGTNYANLTTVEWLRRFNSYPPDNIPDTGTLNVTVNCSCGDSGVGDYGLFVTYPLRPGETLGSVASNVKLDSALLQKYNPNVNFNQGSGIVYIPAKDQNGSYVLLGSSSGGLAGGAIAGIAAGVAVCLLLLAGFIYVGYFRKKRIQKEELLSQETRAIFPQDGKDENPRSTVNETPGPGGPAAMAGITVDKSVEFSYDELATATDNFSLANKIGQGGFGSVYYAELRGERAAIKKMDMQASKEFLAELKVLTRVHHLNLVRLIGYSIEGSLFLVYEFIENGNLSQHLRGSGRDPLPWATRVQIALDSARGLEYIHEHTVPVYIHRDIKSANILIDKNYRGKVADFGLTKLTEVGSSSLPTGRLVGTFGYMPPEYAQYGDVSPKVDVYAFGVVLYELISAKDAIVKTSESITDSKGLVALFEGVLSQPDPTEDLRKLVDQRLGDNYPVDSVRKMAQLAKACTQDNPQLRPSMRSIVVALMTLSSTTDDWDVGSFYENQNLVNLMSGR

SEQ ID NO: 35 - protein, 212 aa, Medicago truncatula
MNLKNGLLLFILFLDCVFFKVESKCVKGCDVALASYYIIPSIQLRNISNFMQSKIVLTNSFDVIMSYNRDVVFDKSGLISYTRINVPFPCECIGGEFLGHVFEYTTKEGDDYDLIANTYYASLTTVELLKKFNSYDPNHIPVKAKINVTVICSCGNSQISKDYGLFVTYPLRSDDTLAKIATKAGLDEGLIQNFNQDANFSIGSGIVFIPGR

SEQ ID NO: 36 - protein, 212 aa, Lotus japonicus
MKLKTGLLLFFILLLGHVCFHVESNCLKGCDLALASYYILPGVFILQNITTFMQSEIVSSNDAITSYNKDKILNDINIQSFQRLNIPFPCDCIGGEFLGHVFEYSASKGDTYETIANLYYANLTTVDLLKRFNSYDPKNIPVNAKVNVTVNCSCGNSQVSKDYGLFITYPIRPGDTLQDIANQSSLDAGLIQSFNPSVNFSKDSGIAFIPGR

SEQ ID NO: 37 - protein, 211 aa, synthetic construct
  REGION 1..211: note = Synthetic Construct
MKLKTGLLLFFILLLGHVCFHVESNCLKGCDLALASYYILPSIQLRNISNFMQSEIVSSNDAITSYNKDKIFDKSGLISYQRLNIPFPCDCIGGEFLGHVFEYSASKGDTYETIANLYYANLTTVDLLKRFNSYDPKNIPVNAKVNVTVNCSCGNSQVSKDYGLFITYPIRPGDTLQDIANQSSLDAGLIQSFNPSVNFSKDSGIAFIPGR

SEQ ID NO: 38 - protein, 9 aa, synthetic construct
  REGION 1..9: note = Synthetic Construct
SIQLRNISN

SEQ ID NO: 39 - protein, 9 aa, synthetic construct
  REGION 1..9: note = Synthetic Construct
FDKSGLISY

SEQ ID NO: 40 - protein, 213 aa, synthetic construct
  REGION 1..213: note = Synthetic Construct
MNLKNGLLLFILFLDCVFFKVESKCVKGCDVALASYYIIPGVFILQNITTFMQSKIVLTNSFDVIMSYNRDVVLNDINIQSFTRINVPFPCECIGGEFLGHVFEYTTKEGDDYDLIANTYYASLTTVELLKKFNSYDPNHIPVKAKINVTVICSCGNSQISKDYGLFVTYPLRSDDTLAKIATKAGLDEGLIQNFNQDANFSIGSGIVFIPGR

SEQ ID NO: 41 - protein, 12 aa, synthetic construct
  REGION 1..12: note = Synthetic Construct
PGVFILQNITTF

SEQ ID NO: 42 - protein, 8 aa, synthetic construct
  REGION 1..8: note = Synthetic Construct
NDINIQSF

SEQ ID NO: 43 - protein, 213 aa, synthetic construct
  REGION 1..213: note = Synthetic Construct
MKLKTGLLLFFILLLGHVCFHVESNCLKGCDLALASYYILPSIQLRNISNFMQSKIVLTNSFDVIMSYNRDVVFDKSGLISYTRLNIPFPCDCIGGEFLGHVFEYSASKGDTYETIANLYYANLTTVDLLKRFNSYDPKNIPVNAKVNVTVNCSCGNSQVSKDYGLFITYPIRPGDTLQDIANQSSLDAGLIQSFNPSVNFSKDSGIAFIPGR

SEQ ID NO: 44 - protein, 42 aa, synthetic construct
  REGION 1..42: note = Synthetic Construct
SIQLRNISNFMQSKIVLTNSFDVIMSYNRDVVFDKSGLISYT

SEQ ID NO: 45 - protein, 213 aa, synthetic construct
  REGION 1..213: note = Synthetic Construct
MKLKTGLLLFFILLLGHVCFHVESNCLKGCDLALASYYILPSIQLRNISNFMQSEIVLTNSFDAITSYNKDKIFDKSGLISYTRLNIPFPCDCIGGEFLGHVFEYSASKGDTYETIANLYYANLTTVDLLKRFNSYDPKNIPVNAKVNVTVNCSCGNSQVSKDYGLFITYPIRPGDTLQDIANQSSLDAGLIQSFNPSVNFSKDSGIAFIPGR

SEQ ID NO: 46 - protein, 6 aa, synthetic construct
  REGION 1..6: note = Synthetic Construct
LTNSFD

SEQ ID NO: 47 - protein, 10 aa, synthetic construct
  REGION 1..10: note = Synthetic Construct
FDKSGLISYT

SEQ ID NO: 48 - protein, 213 aa, synthetic construct
  REGION 1..213: note = Synthetic Construct
MKLKTGLLLFFILLLGHVCFHVESNCLKGCDVALASYYIIPSIQLRNISNFMQSKIVLTNSFDVIMSYNRDVVFDKSGLISYTRINVPFPCDCIGGEFLGHVFEYSASKGDTYETIANLYYANLTTVDLLKRFNSYDPKNIPVNAKVNVTVNCSCGNSQVSKDYGLFITYPIRPGDTLQDIANQSSLDAGLIQSFNPSVNFSKDSGIAFIPGR

SEQ ID NO: 49 - protein, 60 aa, synthetic construct
  REGION 1..60: note = Synthetic Construct
DVALASYYIIPSIQLRNISNFMQSKIVLTNSFDVIMSYNRDVVFDKSGLISYTRINVPFP

SEQ ID NO: 50 - protein, 211 aa, synthetic construct
  REGION 1..211: note = Synthetic Construct
MNLKNGLLLFILFLDCVFFKVESKCVKGCDVALASYYIIPGVFILQNITTFMQSEIVSSNDAITSYNKDKILNDINIQSFQRINVPFPCECIGGEFLGHVFEYTTKEGDDYDLIANTYYASLTTVELLKKFNSYDPNHIPVKAKINVTVICSCGNSQISKDYGLFVTYPLRSDDTLAKIATKAGLDEGLIQNFNQDANFSIGSGIVFIPGR

SEQ ID NO: 51 - protein, 42 aa, synthetic construct
  REGION 1..42: note = Synthetic Construct
PGVFILQNITTFMQSEIVSSNDAITSYNKDKILNDINIQSFQ

SEQ ID NO: 52 - protein, 211 aa, synthetic construct
  REGION 1..211: note = Synthetic Construct
MNLKNGLLLFILFLDCVFFKVESKCVKGCDVALASYYIIPGVFILQNITTFMQSKIVSSNDVIMSYNRDVVLNDINIQSFQRINVPFPCECIGGEFLGHVFEYTTKEGDDYDLIANTYYASLTTVELLKKFNSYDPNHIPVKAKINVTVICSCGNSQISKDYGLFVTYPLRSDDTLAKIATKAGLDEGLIQNFNQDANFSIGSGIVFIPGR

SEQ ID NO: 53 - protein, 4 aa, synthetic construct
  REGION 1..4: note = Synthetic Construct
SSND

SEQ ID NO: 54 - protein, 211 aa, synthetic construct
  REGION 1..211: note = Synthetic Construct
MNLKNGLLLFILFLDCVFFKVESKCVKGCDLALASYYILPGVFILQNITTFMQSEIVSSNDAITSYNKDKILNDINIQSFQRLNIPFPCECIGGEFLGHVFEYTTKEGDDYDLIANTYYASLTTVELLKKFNSYDPNHIPVKAKINVTVICSCGNSQISKDYGLFVTYPLRSDDTLAKIATKAGLDEGLIQNFNQDANFSIGSGIVFIPGR

SEQ ID NO: 55 - protein, 59 aa, synthetic construct
  REGION 1..59: note = Synthetic Construct
DLALASYYILPGVFILQNITTFMQSEIVSSNDAITSYNKDKILNDINIQSFQRLNIPFP

SEQ ID NO: 56 - protein, 222 aa, synthetic construct
  REGION 1..222: note = Synthetic Construct
MVSAIVLYVLLAAAAHSAFACPVDSPPSCDTYVTYFAQSPNFLTLTSISDLFDTSPLSIARASNIKDENQNLVPGQLLLVPVTCACSGSNSFSNISHMIKEGESYYYLSTTSYENLTNWETVQDSNPNYNPYLLPVGIKVVIPLFCKCPSNYHLNKGIEYLITYVWHNNDNVSLVASKFGVSTQDIISENNFSHQNFTAATNFPILIPVTQLPSLSHHHHHH

SEQ ID NO: 57 - protein, 222 aa, synthetic construct
  REGION 1..222: note = Synthetic Construct
MVSAIVLYVLLAAAAHSAFACPVDSPPSCDTYVTYFAQSPNFLTLTSISDLFDTSPLSIARASNIKDENQNLVPGQLLLVPVTCACAGNHSSANTSYQIQLGDSYDFVATTLYENLTNWNIVQASNPGVNPYLLPERVKVVFPLFCKCPSNYHLNKGIEYLITYVWHNNDNVSLVASKFGVSTQDIISENNFSHQNFTAATNFPILIPVTQLPSLSHHHHHH

SEQ ID NO: 58 - protein, 222 aa, synthetic construct
  REGION 1..222: note = Synthetic Construct
MVSAIVLYVLLAAAAHSAFACPVDSPPSCDTYVTYFAQSPNFLTLTSISDLFDTSPLSIARASNIKDENQNLVPGQLLLVPVTCACSGSNSFSNISHMIQLGDSYDYLSTTSYENLTNWETVQDSNPGVNPYLLPVGIKVVIPLFCKCPSNYHLNKGIEYLITYVWHNNDNVSLVASKFGVSTQDIISENNFSHQNFTAATNFPILIPVTQLPSLSHHHHHH

SEQ ID NO: 59 - protein, 20 aa, synthetic construct
  REGION 1..20: note = Synthetic Construct
MVSAIVLYVLLAAAAHSAFA

SEQ ID NO: 60 - protein, 196 aa, synthetic construct
  REGION 1..196: note = Synthetic Construct
CPVDSPPSCDTYVTYFAQSPNFLTLTSISDLFDTSPLSIARASNIKDENQNLVPGQLLLVPVTCACSGSNSFSNISHMIKEGESYYYLSTTSYENLTNWETVQDSNPNYNPYLLPVGIKVVIPLFCKCPSNYHLNKGIEYLITYVWHNNDNVSLVASKFGVSTQDIISENNFSHQNFTAATNFPILIPVTQLPSLS

SEQ ID NO: 61 - protein, 6 aa, synthetic construct
  REGION 1..6: note = Synthetic Construct
HHHHHH

SEQ ID NO: 62 - protein, 59 aa, synthetic construct
  REGION 1..59: note = Synthetic Construct
AGNHSSANTSYQIQLGDSYDFVATTLYENLTNWNIVQASNPGVNPYLLPERVKVVFPLF

SEQ ID NO: 63 - protein, 7 aa, synthetic construct
  REGION 1..7: note = Synthetic Construct
QLGDSYD

SEQ ID NO: 64 - skipped

SEQ ID NO: 65 - protein, 233 aa, synthetic construct
  REGION 1..233: note = Synthetic Construct
MVSAIVLYVLLAAAAHSAFAQGGAGNGTGRFACVVPAPCDTFVLYRTQSPGSLDLGAISDLFGVSRAMIAAANNLSLIDEDAALLPDQPLLVPVRCGCTGNRSFVNVTYPIHSGDTFYALALTGYENLTTPDVIQELNPQAVFNKLNVSQLVTVPLFCRCPTPAERSAGVLQQITYMWRPVDTMSRVSKLMGSDASAIAAANNVSADFTSTTMLPMLIPVARPPVLPHHHHHH

SEQ ID NO: 66 - protein, 207 aa, synthetic construct
  REGION 1..207: note = Synthetic Construct
QGGAGNGTGRFACVVPAPCDTFVLYRTQSPGSLDLGAISDLFGVSRAMIAAANNLSLIDEDAALLPDQPLLVPVRCGCTGNRSFVNVTYPIHSGDTFYALALTGYENLTTPDVIQELNPQAVFNKLNVSQLVTVPLFCRCPTPAERSAGVLQQITYMWRPVDTMSRVSKLMGSDASAIAAANNVSADFTSTTMLPMLIPVARPPVLP

SEQ ID NO: 67 - protein, 228 aa, synthetic construct
  REGION 1..228: note = Synthetic Construct
MVSAIVLYVLLAAAAHSAFAGDGCSRGCDLALASYYIAPNQNVTYIASLFGFSEYRVLGQYNPGVNNLDYVVAGDRLNVSLTCKCLASLSAPASTFLAASIPYKVATGETYLRIADNYNNLTTADWLVATNTYPANNIPDVATVNATVNCSCGDAGISTDYGLFLTYPLRDRETLASVAANHGFSSPEKMDLLKKYNPGMDGVTGSGIVYIPAKDPNGSYRPHHHHHH

SEQ ID NO: 68 - protein, 202 aa, synthetic construct
  REGION 1..202: note = Synthetic Construct
GDGCSRGCDLALASYYIAPNQNVTYIASLFGFSEYRVLGQYNPGVNNLDYVVAGDRLNVSLTCKCLASLSAPASTFLAASIPYKVATGETYLRIADNYNNLTTADWLVATNTYPANNIPDVATVNATVNCSCGDAGISTDYGLFLTYPLRDRETLASVAANHGFSSPEKMDLLKKYNPGMDGVTGSGIVYIPAKDPNGSYRP

SEQ ID NO: 69 - protein, 621 aa, Medicago truncatula
MEHQPRFTSFISLPLFSIFLASIPFITESKCTKGCSLALANFYVSQGSNLTYISSIMRSNIQTRPEDIVEYSREIIPSKDSVQAGQRLNVPFPCDCIDGQFLGHKFSYDVETGDTYETVATNNYANLTNVEWLRRFNTYPPNDIPDTGTLNVTVNCSCGDADVGNYALFVTYPLRPGETLVSVANSSKVDSSLLQRYNPGVNFNQGSGIVFVPGKDQNGSFVFLGSSSGLGGGAIGGIAVGIVVVLLLVAAAIYFGYFRKKKIQKEELFSRDSTALFSQDGKDENSHGAANVTQRPGVMTGITVDKSVEFSYDELAAASDNFSMANKIGQGGFGSVYYAELRGEKAAIKKMDMQATKEFLAELKVLTRVHHLNLVRLIGYSIEGSLFLVYEYIENGNLSQHLRGSGRDPLPWATRVQIALDSARGLEYIHEHTVPVYIHRDIKPANILIDKNFRGKVADFGLTKLTEVGSSSLPTGRLVGTFGYMPPEYAQYGDVSPKVDVYAFGVVLYELISAKEAIVKSSESVADSKGLVGLFEGVLSQPDPTEDLRKIVDPRLGDNYPADSVRKMAQLAKACTQENPQLRPSMRSIVVALMTLSSTTDDWDVGSFYENQNLVNLMSGR

SEQ ID NO: 70 - protein, 623 aa, Lotus japonicus
MKLKTGLLLFFILLLGHVCFHVESNCLKGCDLALASYYILPGVFILQNITTFMQSEIVSSNDAITSYNKDKILNDINIQSFQRLNIPFPCDCIGGEFLGHVFEYSASKGDTYETIANLYYANLTTVDLLKRFNSYDPKNIPVNAKVNVTVNCSCGNSQVSKDYGLFITYPIRPGDTLQDIANQSSLDAGLIQSFNPSVNFSKDSGIAFIPGRYKNGVYVPLYHRTAGLASGAAVGISIAGTFVLLLLAFCMYVRYQKKEEEKAKLPTDISMALSTQDGNASSSAEYETSGSSGPGTASATGLTSIMVAKSMEFSYQELAKATNNFSLDNKIGQGGFGAVYYAELRGKKTAIKKMDVQASTEFLCELKVLTHVHHLNLVRLIGYCVEGSLFLVYEHIDNGNLGQYLHGSGKEPLPWSSRVQIALDAARGLEYIHEHTVPVYIHRDVKSANILIDKNLRGKVADFGLTKLIEVGNSTLQTRLVGTFGYMPPEYAQYGDISPKIDVYAFGVVLFELISAKNAVLKTGELVAESKGLVALFEEALNKSDPCDALRKLVDPRLGENYPIDSVLKIAQLGRACTRDNPLLRPSMRSLVVALMTLSSLTEDCDDESSYESQTLINLLSVR

SEQ ID NO: 71 - protein, 620 aa, Medicago truncatula
MNLKNGLLLFILFLDCVFFKVESKCVKGCDVALASYYIIPSIQLRNISNFMQSKIVLTNSFDVIMSYNRDVVFDKSGLISYTRINVPFPCECIGGEFLGHVFEYTTKEGDDYDLIANTYYASLTTVELLKKFNSYDPNHIPVKAKINVTVICSCGNSQISKDYGLFVTYPLRSDDTLAKIATKAGLDEGLIQNFNQDANFSIGSGIVFIPGRDQNGHFFPLYSRTGIAKGSAVGIAMAGIFGLLLFVIYIYAKYFQKKEEEKTKLPQTSRAFSTQDASGSAEYETSGSSGHATGSAAGLTGIMVAKSTEFTYQELAKATNNFSLDNKIGQGGFGAVYYAELRGEKTAIKKMDVQASSEFLCELKVLTHVHHLNLVRLIGYCVEGSLFLVYEHIDNGNLGQYLHGIGTEPLPWSSRVQIALDSARGLEYIHEHTVPVYIHRDVKSANILIDKNLRGKVADFGLTKLIEVGNSTLHTRLVGTFGYMPPEYAQYGDVSPKIDVYAFGVVLYELITAKNAVLKTGESVAESKGLVQLFEEALHRMDPLEGLRKLVDPRLKENYPIDSVLKMAQLGRACTRDNPLLRPSMRSIVVALMTLSSPTEDCDDDSSYENQSLINLLSTR

SEQ ID NO: 72 - protein, 617 aa, Zea mays
MARILMRLLLLAAAAAVAAGDGCLNSGCVALGSYLVARNQNLTYIASLFGIGDYHALARYNPGTTNLDYIQAGQSVNISFTCGCHTFPNSDATYLGGSFPHKVVTGDTYGGIAQNYNNLTSAAWLAVTNPYPTNNIPDTNTVVNVTVNCTCGDPKISSDYGFFLTYPLMGQTLAAVAANYSFNSSSQLDLLRKYNPGMDTATSGLVFIPVKDGNGSYHPLKPPGNGGSIGAIVGGVVGGVAILVLGVLLYIMFYRRKKANKAALLPSSEDSTQLATTSMDKSALSTSQADSSSGVPGITVDKSVEFSYEELFNATEGFSMSNKIGQGGFGAVYYAELRGEKAAIKKMDMQASHEFLAELKVLTHVHHLNLVRLIGFCTESSLFLVYEFIENGNLSQHLRGTGYEPLSWAARVQIALDSARGLEYIHEHTVPVYIHRDIKSANILIDKNYRAKVADFGLTKLTEVGNTSLPTRGIVGTFGYMPPEYARYGDVSPKVDVYAFGVVLYELISAKDAIVRSTESSSDSKGLVYLFEEALNTPDPKEGLQRLIDPALGEDYPIDSILKMTVLARACTQEDPKARPTMRSIVVALMTLSSTSEFWDMNAIQENQGVVNLMSGR

SEQ ID NO: 73 - protein, 626 aa, Hordeum vulgare
MEAPLHSLLLLLLLAAAAGPKTAAAVGDGCSRGCDLALASYYIAPNQNVTYIASLFGFSEYRVLGQYNPGVNNLDYVVAGDRLNVSLTCKCLASLSAPASTFLAASIPYKVATGETYLRIADNYNNLTTADWLVATNTYPANNIPDVATVNATVNCSCGDAGISTDYGLFLTYPLRDRETLASVAANHGFSSPEKMDLLKKYNPGMDGVTGSGIVYIPAKDPNGSYRPLESPGKKSSAGAIAGGVVAGVVALVLGVVLFLFYRRRKAKKDALLPSSEESTRLASAISMQKVTPSTSQADGASPAAGITVDKSVEFSYEELFNATEGFNIIHKIGQGGFGAVYYAELRGEKAAIKKMDMQATQEFLAELKVLTHVHHLNLVRLIGYCTESSLFLVYEFIENGNLSQHLRGTGYEPLSWVERVQIALDSARGLEYIHEHTVPVYIHRDIKSANILIDKNTRAKVADFGLTKLTEVGGGTSLQTRVVGTFGYMPPEYARYGDVSPKVDVYAFGVVLYELISAKDAIVRSAESTSDSKGLVYLFEEALSAPDPKEGIRRLMDPKLGDDYPIDAILKMTHLANACTQEDPKLRPTMRSVVVALMTLSSTSEFWDMNALYENPGLVNLMSGR

SEQ ID NO: 74 - protein, 613 aa, Oryza sativa
  source 1..613: note = Japonica Group
MFSLPALLIGACAFAAAAVAASGDGCRAGCSLAIAAYYFSEGSNLTFIATIFAIGGGGYQALLPYNPAITNPDYVVTGDRVLVPFPCSCLGLPAAPASTFLAGAIPYPLPLPRGGGDTYDAVAANYADLTTAAWLEATNAYPPGRIPGGDGRVNVTINCSCGDERVSPRYGLFLTYPLWDGETLESVAAQYGFSSPAEMELIRRYNPGMGGVSGKGIVFIPVKDPNGSYHPLKSGVGIVLLFCGMGNSLSGGAIAGIVIACIAIFIVAIWLIIMFYRWQKFRKATSRPSPEETSHLDDASQAEGIKVERSIEFSYEEIFNATQGFSMEHKIGQGGFGSVYYAELRGEKTAIKKMGMQATQEFLAELKVLTHVHHLNLVRLIGYCVENCLFLVYEFIDNGNLSQHLQRTGYAPLSWATRVQIALDSARGLEYLHEHVVPVYVHRDIKSANILLDKDFRAKIADFGLAKLTEVGSMSQSLSTRVAGTFGYMPPEARYGEVSPKVDVYAFGVVLYELLSAKQAIVRSSESVSESKGLVFLFEEALSAPNPTEALDELIDPSLQGDYPVDSALKIASLAKSCTHEEPGMRPTMRSVVVALMALTANTDLRDMDYHPF

SEQ ID NO: 75 - protein, 617 aa, Arabidopsis thaliana
MKLKISLIAPILLLFSFFFAVESKCRTSCPLALASYYLENGTTLSVINQNLNSSIAPYDQINFDPILRYNSNIKDKDRIQMGSRVLVPFPCECQPGDFLGHNFSYSVRQEDTYERVAISNYANLTTMESLQARNPFPATNIPLSATLNVLVNCSCGDESVSKDFGLFVTYPLRPEDSLSSIARSSGVSADILQRYNPGVNFNSGNGIVYVPGRDPNGAFPPFKSSKQDGVGAGVIAGIVIGVIVALLLILFIVYYAYRKNKSKGDSFSSSIPLSTKADHASSTSLQSGGLGGAGVSPGIAAISVDKSVEFSLEELAKATDNFNLSFKIGQGGFGAVYYAELRGEKAAIKKMDMEASKQFLAELKVLTRVHHVNLVRLIGYCVEGSLFLVYEYVENGNLGQHLHGSGREPLPWTKRVQIALDSARGLEYIHEHTVPVYVHRDIKSANILIDQKFRAKVADFGLTKLTEVGGSATRGAMGTFGYMAPETVYGEVSAKVDVYAFGVVLYELISAKGAVVKMTEAVGEFRGLVGVFEESFKETDKEEALRKIIDPRLGDSYPFDSVYKMAELGKACTQENAQLRPSMRYIVVALSTLFSSTGNWDVGNFQNEDLVSLMSGR

SEQ ID NO: 76 - protein, 620 aa, Lotus japonicus
MFYDFTTMASLTHPLCVLLTLMAAASFASVFSLEVSSKTTYMEPFNCSTKIRTCNSLLYHISIGLKVEEIARFYSVNLSRIKPITRGTKQDYLVSVPCTCRNTNGLNGYFYHTSYKVKVNDSFVDIQNLFYSGQAWPVNEDLVVPNETMTIHIPCGCSESGSQIVVTYTVQRNDTPLSIALLLNATVEGMVSVNSVMAPNPTFIDVGWVLYVPKELNPISHGKENKHKLEKIIGILAGVILLSIITLIILIVRRNRSYETCKDDPRAISKRSIGKRTSSLMNRDFHKEYMEDATSFDSERPVIYTLEEIEQATNDFDETRRIGVGGYGTVYFGVLGEKEVAIKKMKSNKSKEFYAELKALCKIHHINIVELLGYASGDDHLYLVYEYVPNGSLSEHLHDPLLKGHQPLSWCARIQIALDSAKGIEYIHDYTKAQYVHRDIKTSNILLDEKLRAKVADFGLAKLVERTNDEEFIATRLVGTPGYLPPESLKELQVTVKTDVFAFGVVMLELITGKRALFRDNQEANNMRSLVAVVNQIFQEDNPETALEVTVDGNLQRSYPMEDVYNMAELSHWCLRENPVDRPEMSEIVVKLSKIIMSSIEWEASLGGDSQVFSGVFDGR

SEQ ID NO: 77 - protein, 613 aa, Medicago truncatula
MASLIQLLSIFLPLLASSLPTIFSIEVSMKKAYMEPYKCSTKMRTCNASLYHINYNHNIEQIANFYSIDPSQIKPIIRSTKQDYLVKVPCSCKNIKDLSGYFYETTYKVSPNETSVDIMNLIYSGQAWQVNEDLVANENVTIHIPCGCSEFESQIVVTYTVQQSDTPTSISLLLNATIDGMVRINQILGPNPTFIDIGWVLYVPKELKGSPLYHGKEKKHKWVIIIGILVSVTLLSVITLIIFILRRNKAYETSKYDPKTVSKRSFGNRTISLRNHEFHKEYMEDATQFDSERPVIYDFEEIEHATNNFDETRRIGVGGYGTVYFGMLEEKEVAVKKMKSNKSKEFYAELKALCKIHHINIVELLGYASGDDHLYLVYEYVPNGSLSEHLHDPLLKGHQPLSWCARTQIALDSAKGIEYIHDYTKARYVHRDIKTSNILLDEKLRAKVADFGLAKLVERTNDEEFLATRLVGTPGYLPPESVKELQVTIKTDVFAFGVVISELITGKRALFRDNKEANNMKSLIAVVNKIFQDEDPVAALEAVVDGNLLRNYPIEGVYKMAELSHWCLSEEPVDRPEMKEIVVAVSKIVMSSIEWEASLGGDSQVFSGVFDGR

SEQ ID NO: 78 - protein, 595 aa, Lotus japonicus; identical to SEQ ID NO: 2

SEQ ID NO: 79 - protein, 632 aa, Medicago truncatula
MKTLKPHHHQLSTFIFILLFPFLKSQTARQQNNTGYTCPNNNNNNNNTYPCQTYVYYKATPPNYLDLATISDLFQLSRLMISKPSNISSPSSPLLPNQPLLIPLTCSCNFINTTFGSISYSNITYTIKPNDTFFLVSTINFQNLTTYPSVQVVNPNLVATNLSIGDNAVFPIFCKCPDKTKTNSSFMISYVVQPHDNVSSIASMFGTSEKSIVDVNGERLYDYDTIFVPVTELPVLKQPSTIVPSPAPRGNSDDGDDDDDKSGIVKGLAIGLGILGFLLILVIVFWFYREVLFKKEKKGKGLYFGDKGYKGNDEKKKKMDVNFMANVSDCLDKYRVFGFDELVEATDGFDERFLIQGSVYKGEIDGQVYAIKKMKWNAYEELKILQKVNHGNLVKLEGFCIEPEESNCYLVYEYVENGSLYSWLHEDKNEKLNWVTRLRIAVDIANGLLYIHEHTRPKVVHKDIKSSNILLDSNMRAKIANFGLAKSGINAITMHIVGTQGYISPEYLADGIVSTKMDVFSFGIVLLELISGKEVIDEEGNVLWASAIKTFEVKNEQEKARRLKEWLDRTMLKETCSMESLMGVLHVAIACLNRDPSKRPSIIDIVYSLSKCEEAGFELSDDGFGSERLVAR

SEQ ID NO: 80 - protein, 423 aa, Arabidopsis thaliana
MKNPEKPLLLFLILASSLASMATAKSTIEPCSSKDTCNSLLGYTLYTDLKVTEVASLFQVDPVSMLLSNSIDISYPDVENHVLPAKLFLKIPITCSCVDGIRKSLSTHYKTRTSDTLGSIADSVYGGLVSPEQIQVANSETDLSVLDVGTKLVIPLPCACFNGTDESLPALYLSYVVRGIDTMAGIAKRFSTSVTDLTNVNAMGAPDINPGDILAVPLLACSSNFPKYATDYGLIIPNGSYALTAGHCVQCSCVLGSRSMYCEPASISVSCSSMRCRNSNFMLGNITSQQSSSGCKLTTCSYNGFASGTILTTLSMSLQPRCPGPQQLAPLIAPPDNVPKELMYLPSPSPSPSPEFDDIAGGGSSIAAVPAASPGGATVSSSNSIPGNPANGPGGSISIASCPLSYYSFIALLIPIGSCFFVF

SEQ ID NO: 81 - protein, 382 aa, Oryza sativa
  source 1..382: note = Japonica Group
PAPVSTRQSNKHQASHSHLSRRAFPTMASLTAALATPAAAALLLLVLLAAPASAANFTCAVASGTTCKSAILYTSPNATTYGNLVARFNTTTLPDLLGANGLPDGTLSSAPVAANSTVKIPFRCRCNGDVGQSDRLPIYVVQPQDGLDAIARNVFNAFVTYQEIAAANNIPDPNKINVSQTLWIPLPCSCDKEEGSNVMHLAYSVGKGENTSAIAAKYGVTESTLLTRNKIDDPTKLQMGQILDVPLPVCRSSISDTSADHNLMLLPDGTYGFTAGNCIRCSCSSTTYQLNCTAVQNKGCPSVPLCNGTLKLGETNGTGCGSTTCAYSGYSNSSSLIIQTSLATNQTTACQRGGSGRSQFARSMWSMSVISFHMVLIIICFL

SEQ ID NO: 82 - protein, 350 aa, Arabidopsis thaliana
METSCFTLLGLLVSLSFFLTLSAQMTGNFNCSGSTSTCQSLVGYSSKNATTLRNIQTLFAVKNLRSILGANNLPLNTSRDQRVNPNQVVRVPIHCSCSNGTGVSNRDIEYTIKKDDILSFVATEIFGGLVTYEKISEVNKIPDPNKIEIGQKFWIPLPCSCDKLNGEDVVHYAHVVKLGSSLGEIAAQFGTDNTTLAQLNGIIGDSQLLADKPLDVPLKACSSSVRKDSLDAPLLLSNNSYVFTANNCVKCTCDALKNWTLSCQSSSEIKPSNWQTCPPFSQCDGALLNASCRQPRDCVYAGYSNQTIFTTASPACPDSAGPDNYASTLSSSFNFVIVLIQCALLCLCLL

SEQ ID NO: 83 - protein, 632 aa, Medicago truncatula; identical to SEQ ID NO: 79

SEQ ID NO: 84 - protein, 647 aa, Zea mays
MRPPALRLSALLLFLCLYAAPARSQNATTVTAAPASVEGFNCSVNRTYPCQAYALYRAGFAGVPLNLAAIGDLFAASRFMVAHANNLSTAAAPATGQPLLVPLQCGCPSGSPNSYAPMQYQIASGDTYWIISTTKLQNLTQYQAVERVNPTLVPTNLDVGTMVTFPIFCQCPAAADNATALVTYVMQPGDTYSTIAAAFSVDAQSLVSLNGPEPRTQQFAEILVPLRRQVPGWLPPIVLRNNASATPAAPPPSASPNATVVRNDRNGVVTGLAVGLGVVGALWLLQMLLLACLCRRLRANGRRGDAVLSGDGVEGGVFAKGSSAAAAGGGERFLVSDMSEWLDKYRVFTVEELERGTGGFDDAHLVNGSVYKANIDGLVFAVKKMKWDACEELKILQKVNHSNLVKLEGFCIDSATGDCYLVYEYVENGSLDLWLLDRDHARRLNWRARLHIALDLAHGLQYIHEHTWPRVVHKDMKSSNVLLDARMRAKIANFGLAKTGHNAITTHIVGTQGYIAPEYLADGLVTTKIDVFAYGVVLLELVSGREAADESGEPLWADAEDRVFRGRDERLEARVAAWMDPALAEQTCPLGSVATVVSVARACLHKDPSKRPSMVDVAYTLSKADEHFADYSGESVSVDGSGEIAAR

SEQ ID NO: 85 - protein, 680 aa, Zea mays
MAPPPQPELPAAALALLVLLLAAAVPARAQQEYEANKQNACYATNASSVLGYTCNATAASTPACESYLIFRSSPSYYNTPVSISYLLNSSPATVAAANAVPTVSPLAASSLVLVPVPCACTPGGYYQHNSSYTIEFQSETYFIIANITYQGLTTCQALIAQNPLHDSRGLVAGNNLTVPLRCACPSPAQAAKGFRYLLSYLVMWGDGVPSIAARFRVDPQAVLDANSLTADDIIFPFTTLLIPLKAAPTPDMLASPAPPPSPTPPQPTPAPSGGSGSGKWVGVGVGLGCGALALAAILGLLFLRTRRRRGQRFADGESVRQGSKVVIDVSSSAEYGALASGKQTSNTTTSTTSSATRSLVASDVRGAVESLTVYKYSELEKATAGFAEERQVPGTSVFRAVINGDAAAVKLVAGDVRDEVSILMRVNHSCLVRLSGLCVHRGDTYLVFEFAENGALSDWIHGGGGSTLRWRQRVQVAFDVADGLNYLHHYTNPPCVHKNLKSSNVLLDADLRAKVSSFGLARTVAASDGGAQLTRHVAGTQGYLAPEYLEDGLITPKLDVFAFGVVLLELLSGKEAAFADAGTGEETLLWEAAEEALVADGGEDVDRAKVRAFMDPRLHGDFPIDLALAMAALALRCVATEPRARPAMDEVFVSLTAVHNSTLDWDPSDYGTSGSSMVGR

SEQ ID NO: 86 - protein, 684 aa, Zea mays
MMASPPQTELQAVALALLVLLLAGAAPARAQQEYEANKQNACYATNASSVLGYTCNATTASTPACDSYLIFRSSPTYYNTPVSISYLLNSSVSATAAANAVPSVSPLAPSSLVLVPVPCACTPGGYYQHNSSYTIQFRGETYFIIANITYQGLTTCQALIAHNPLHDSRGLVAGNNLTVPLRCACPSPAQAAKGFKYLLSYLIMWGDDVTSIAARFRADPQAVLDANSLTADDIIFPFTTLLIPLKTAPTLDMLASTAPPPAPTPPQPAPAPSGRSGSGKLVGFGVGLGCGALALAGILGLLFLRARRRQRLPVGESVRQGSKVVIDVSSSADYGALASGKKITNTTTSSMSSAAWSLVASDVRGAVESLTVYKYSELEKATAGFAEEHQVPGTSVYRAVINGDAAAVKRLAGDVSGEVGILMRVNHSCLVRLSGLCVHRGDTYLVFEFAENGALSDWIHGGSGSCSGSNTLRWRQRVQVAFDIADGLNYLHHYTNPPCVHKNLKSSNVLLDADLRAKVSGFGLARAVTAAHGGAQLTGHVVGTQGYLAPEYLEDGLITPKLDVFAFGVVLLELLSGKEAGFADAGTGEEILLCESAEEALVADGGEDMDRAKVRAFMDPRLHGDFPMDLALSMAALALRCVAMEPRARPAMDEVFISLSAVYNSTMDCDPSDYGTSGSSMIGR

SEQ ID NO: 87 - protein, 723 aa, Zea mays
MNFSTWKTKEVQVRSFTTRKPTPQAACAMASFHDLTATAVLLLLFSILSGGLAPLQVQAQQPYGSQIADCTNQHNSSSLLGYFCGAAGSAPSCPTFLTFTARAQYSSLATIGALLGADPASVLAPNEATGADAPLPAGTRVLVPATCACTATPGGRFYQRNATYVAVAGDTLLIIANNTFQGLTSCQALEAQALRGAPPQSLDVGQSLPVPLRCACPSAAQAAAGARYLVSYLVDVFDDLTTVAARFGVDMGTVAASNQLQPPFTIDPYTTLLIPVSAQPNVSRIQTPPSPPPPPPVVARAPAPGKKSSNHVGVYIGVAVAVVVVAAIVSAGAFLAVRARRRRAGAVLATGEVAKKESKAGNDRAATSSGFTGGEFSLSTSEAFSSISVTDIKSSLKVYTYAELKAATDDFSPEHRIGGSVYRAAFNGDAAAVEVVDRNVSTEVEIMRKINHLNLIRLIGLCHHRGRWYLVTEYAEHGALRDRLLASATGTAAPLTWAQRVHIALDVAEGLRYLHEYARPAWVHMDVSSGSVLLAGDGPRAKLRGFGAARAITGATAGVDGEEGAEEALFTMTSRIAGTRGYIAPEYLEHGVVSPKADVYSLGVVLLELVTGRDAEELVGDGVGDPFVALRELAEELDGGGDAVLQRLEELVDPALPAGSCPQDAVVMVVRLIERCVRQDPARRPTTGEVAQRLLKLSGVSVVSWRNSPESPRSSGSGKGLMY

SEQ ID NO: 88 - protein, 587 aa, Zea mays
MTKHGLLFFLAIALLLHRHYTSASSTGQPTRSSSTASRSTAEWQPLHCSPVSSCGSFLYVTPGGRNLSEIASVFNGNASLIQPVKRLSGSEDLLMAVACECQAISNTTTAAAFLHDTQYKVEPDAIPDDVKSNTFSGLAMDVGDGFPLTPGATVTVRLPCGCSSSTASKGVLSYSVQEEDTLSTIASLFSSSPEAILNLNPSVKNPDFIKPGWILFVPMGVAGSSKKKRVGSTTITIAASVSAIILSVCVLTVILRLRRRPSQQNAEAPEIKMERAPNIDPFQTERPVIFSLKVVGDATANFDEKRKIGEGGYGSVYLGFIGTHEIAVKKMRASKSKEFFAELKALCKVHHINVVELIGYAAGDDHLYLVYEYVQNGSLSEHLHDPLLKGHQPLSWTARTQIALDAARGIEYIHDHTKACYVADFGLVKLVERSDEEEWVATRLVGTPGYLPPESVLELHMTTKSDVYAFGVVLAELITGLRALIRDNKEVNKTKSIISIMRKAFDSEDLERSLETIIDPNLKDSYPIEEVCKMANVSMWCLSEDPLNRPEMRDTMPALCQIHLASIEWEASLGGDGEVFSGVSYGR

SEQ ID NO: 89 - protein, 629 aa, Zea mays
MLYPHSAQQTRTAWTTRSRSRMDTACHHYTSASFTDQPSSIASAAEWQPLTCNAAVSNNPSCGSFLYVTPRGRTLSEVVSVFNGNASLIQPIKRLSGSEDLLVGVACKCEAINDTMTAFFHDTQYEVEPGDTPDNVKSNNFSGLAMNVGDGRTLIAGTTIAVHLPCGCSSTAPEGVLSYSVQEEDTLSTIASLFSSRQQDILNLNPILRNADFIRTGWILFIPMGVAGSSKKGIGSMRIIIAASVSAAVLLFCVLAVILRRRRRSSQHNVEAPEIKMERAPSNTSIAALESRFFPTMRTNDTDPFQTERPVIFSLKQVGDATADFSEKRKIGEGGYGSVYLGFIGAHEIAIKKMKASKSKEFFAELKALCKVHHINVVELIGYAAGDDHLYLVYEYVQNGSLTDHLHDPLLKGHQPLSWTARTQIALDAARGIEYIHDHTKACYVHRDIKTSNILLDNGLRAKVADFGLVKLVERSDEEEFVATRLVGTPGYLPPESVLELHMTTKSDVYAFGVVLAELITGLRALIRDNKEVNKTKSITSIMREVFKSEDLERSLETIIDPNLKDSYPIEEVCKMANVSMWCLSEDPLNRPETRDIMSTLGQIHLASIEWEASLCGDGEVFSGVSYGR

SEQ ID NO: 90 - protein, 612 aa, Hordeum vulgare
MARHSLFFFFLALSLQHLHISVGLPGRSLATATEQWQPMQCDAASLNASCSSYLYVTPQGRSLSEIASLFNGSASRTQPIKRLSGSEDLLVPVPCMCDAINDNMSGLFHDTAYKVNLNDTADNINSIFSGLAWNITATANTTITVHLLCGCSSTAPEGVISYMVQARDTLSNIATLFRSGSSEILSLNAGVTDPDFLQPGWILFIPMGVASSSKRKFGGLPIIIAVSISAAIMLLCTLTIVLRLRRRSLVPNAEVPKKEMERVPSNTSIAILESRYFPSKRIDDIDPFQTERPVIFSLKAVGEATANFDEKRKIGEGGYGMVYLGFIGTHEIAVKMMKDSKSKEFFAELKVLCKVHHINVVELIGYASGEDHLYLVYEYVQNGSLSEHLHDPLLKGHQPLSWTARTQIATDAARGIEYIHDHTKACYVHRDIKTSNILLDDGLRAKVADFGLVKLVERSDEEDCLATRLVGTPGYLPPESVRELHMTTKSDVYAFGVVLAELITGLRALVRDNKEANKTKSLISTMRKAFKSEDVESSLENIIDPSLKDNYPIEEVCKLANISMWCLSEDPLDRPEMREIMPMLSRIHLTSIEWEASLGGDHEVFSGVFNGR

SEQ ID NO: 91 - protein, 673 aa, Zea mays
MPPHRLLPLLLLLLPLGVSGAAAAGGNATSAPLPCSELSRVCTAFIAFPTAGAGPANATVLESMFDAAPGDLTADAAASPRYAFVRKNCSCLPSRTYLANTTYTIPSSATTSFPNTTAADVAAAAYSGLAVPPPGGAAQRPPRPGAVVALHLLCGCSSGPWNYLLTYVGVEGDTVESLSSRFGASMDAIETANAMAGPDPITAGKVYYIPLNSVPGQAYVTLPAPPAPAPAPTDYTLSGTPDYHSSKFPYGWVIGSMGVALALIVIAVLALVLWKFFGYKPQDRNGQRKSPDRHKFQLLKSGSFCYGSGRYLCCQFGNAKPTRADGGEHHINVPKGVAADVFDREKPIVFTHEEILISTDSFSDANLLGHGTYGSVYYGVLREQEVAIKRMMATKTKEFIVEMKVLCKVHHASLVELIGYAAGKDELFLVYEYSQNGSLKNHLHDPERKGCSSLSWIFRVQIALDAARGLEYIHEHTKDHYVHRDIKSSNILLDGSFRAKISDFGLAKLVVKSNDAEASVTKVVGTFGYLAPEYLRDGLATTKSDVYAFGVVLFELISGKEAITRAEGMGASSNSERCSLASVMLAAVRKCPNSTYMGNLKDCIDHNLRDLYPYDCAYKMAMLAKQCVDEDPVLRPDMKQVVITLSQILLSSIEWEATQAGNSQVFSGLVAGR

SEQ ID NO: 92 - protein, 722 aa, Hordeum vulgare
MPPRRRLLLLLLALACSGGGAAVDTAPGNGTSSPLACSELSRVCTAFLAFPAAGNASVLQSMFDASPGDLTSDPAASPGYAFVRKNCSCLASRTYLANTTYTIPSTVPLNATAAQVAAAAYGGLAVPPPGGALQRPPRPGAVVALHLICGCSSGPWNYLLSYVGSDGDTVESLSSRFGASMDAIEAANGMPGPDPITTGKVYYIPLNSVPGQPYVAMSSAPVPAPAPTQNTLSEISDHHSAKFPYGWVIGGMGVALALIAIALLALLMCKSFQYNHQGSNNQGKSPDQPMPHNFQLLKSGSFCYGSGRYFCCQFGNAKQSRKGGEDHHINVPKGMVVDVFDREKPIVFTYEEILASTDLFSDANLLGHGTYGSVYYGVLRDQEVAIKRMTSTNTKEFIVEMKVLCKVHHASLVELIGYAASKDELFLVYEYSQKGSLRNHLHDPQSKGYTSLSWIYRVQIALDAARGLEYIHEHTKDHYVHRDIKSSNILLDGSFRAKISDFGLAKLGLRSNDAEASVTKVVGTFGYLAPEYLRDGLATAKCDVYAFGVVLFELISGKEAITKADAVGASSNSERRSLASVVSFLTCTQAVIQSTACVFAVISLPKVYIGISSTSFYTSNLKDLSRFGLTGQMLTALRNCHDPTCVGSLKDCIDPNLMDLYPHDCIYQMAMLAKQCADEDPVLRPDMKQAVITLSQILLSSIEWEATLGGNSQVFSGLVAGR

SEQ ID NO: 93 - protein, 613 aa, Hordeum vulgare
MPPPPAYHHLLLLLLLLLRLHGAIASATGFTCTKPSTCQSAVIGYVVPNTITYKELISQFSPTTLHDVVAANQLPFNTATKQVIPPKTTLTIPFRCRCTGNGIGQSGLYIAQNKLDDGLATYQPEIVSSKSIADNAANWKGWIPLPCSCDGADVTHFPYIVRSGDSALAIAAKYGVLLSVLLEINNITNHASLYQGQVLDIPLQGKVGEELSSMGRWSRVYYISGYRKRRLGWFNSAAAEQSAHAAAEAVAATKEAAENSHYSPEAADTVVKRVLVLLLIIIILSLLYFTFYYWKSACESLSSRTNGVIQFYYSDLARATHRFSKESKIGEGQYGTVYKATIKGHEMAVKKLKAEGETKELHRELQTISNTKHTNLVSLKGWCGRLRLIDGKSCWKRQIKVELLLVFEWIPNGNLADHLHNREQVLSWEKRYKIVKGIGSALRYLHHECKPSILHRDIKPDNILLDYHFNAKLADFGLSMITDQNGATVFTIAIGPRRYMDPQLMKEGEFRFNHKSDIYSFGIVLLEIACTGKSRENILHILGGGSGQHVQVDGLADHRLSIFDRTEMARVVVLGLQCSHPDERQRPSMYMAMRFLEEGIELPIASHNRRERL

SEQ ID NO: 94 - protein, 416 aa, Arabidopsis thaliana
MKIPEKPIFLIFVSLILASSLTFTATAKSTIEPCSSNDTCNALLGYTLYTDLKVSEVASLFQVDPISILLANAIDISYPDVENHILPSKLFLKIPITCSCVDGIRKSVSTHYKTRPSDNLGSIADSVYGGLVSAEQIQEANSVNDPSLLDVGTSLVIPLPCACFNGTDNSLPAVYLSYVVKEIDTLVGIARRYSTTITDLMNVNAMGAPDVSSGDILAVPLSACASKFPRYASDFGLIVPNGSYALAAGHCVQCSCALGSRNLYCEPASLAVSCSSMQCRNSNLMLGNITVQQTSAGCNVTTCDYNGIANGTILTMLTRSLQPRCPGPQQFAPLLAPPDTVPRDVMYAPAPSPDFDGPGSIASSPRSSMLPGGGILPGNPANGPAGSISTASASSVSYFFITFLISIASFSLALSS

SEQ ID NO: 95 - protein, 415 aa, Lotus japonicus
MRMQQQQSTLLFLVVLLFHSLTTTTCKSTIEPCTNSDSCNALLGYTLYTDLKVSEVASLFGIDPISLLTANAIDISYPDAEHHILPPKLFLKIPISCSCVDGIRKSVSTSYKTRPSDTLSSIADSVYGGLVSADQLTDPSVLDVGQSLVVPLPCTCFNGSDNSLPAIYLSYVVQPVDSLAAIAARYLTTLTDLMNVNAMGSTAISDGDILAVPIPACASNFPKSASDFGLLVPNGSYAITAGHCVQCSCGPRNLNLYCMPTSLSASCSSMQCKNSNLMLGNVTAQQSSAGCNVSSCSYDGLVNGTIATTLSASLQPRCPGLQEFPPLVAPPTSVEKDPTFASGPAPSPSPQSHGSGLPSPKSSGMPGLPGFSPANGPVSGISSGASAACSLVKPSPTLTSALVLLLAMLVIPVAL

SEQ ID NO: 96 - protein, 394 aa, Lotus japonicus
MRNHLQFLWRHILVFLLLSVSYQVEAKSTIEPCSSGFPCPSLLSYILPWDSKLSEIATRFSVNVSNILAANSVFPITPSSGHQILSAKSIVKIPFSCPCVDGIRRSISTIYNVEASDTLASISEGYGGLVSAEQIKTMNSINETNPLTYGSSIVIPLPCKCLNNVNNGDTTVYMSYVVQKGQSLGSIATMYGTTVSDLESVNGLGQNAVDPGDILSVPVAACSSATLNWYSENLIVPNGSYILTASNCIQCTCTPRDLKMECLPSGMDVPCYNLHCKGSNLIIGNEYVEHSQTSCNVSQCVYRGHRGGKILSSIINSSYLQCPDNQSYSGPSRWPSLTPYAAEYPFDISPSPSSPPLPVSEAALRTRASGGWQGQSLINVMQLFLIKLILYFIM

SEQ ID NO: 97 - protein, 370 aa, Lotus japonicus
MGTVWLSKLVATTMLVAVLGLLAEAQIEAKFKCISENAPCHALADYSHPNGTTLRRIQTLFTVKYLPDILGANNLPANTTRVAPDQVIKVPFPCRCSNGTGLSNKVPRYKIKKGDTLYDIATTVFAGLVKYPQIQVANEIPDANNITAGDTIWIPLPCSCDAVAGSSVVHYAHLVQDGSSVESIAQEYGSTQQILLSLNGISDPKLLQARQLLDVPLQACSSSVKNDSPDYPLLVPNATYVYTAKECVKCKCDSSNNFRLQCEPSQHKPINDWSVCPSMECSKNVLIGNTTSTDSCNRTICDYAGYSNSKISTILATQNTCAVPPSGSGTSSGSGSGDSGSGASRSNLHGWVWSSPLIVIHFLLFVVFLL

SEQ ID NO: 98 - protein, 211 aa, Hordeum vulgare
SVEGFNCSANGTYPCQAYALYRAGLAGVPPDLSAAGDLFGVSRFMLAHANNLSTSAAPAAGQPLLVPLQCGCPSGSPNAYAPTQYQISSGDTFWIVSVTKLQNLTQYQAVERVNPTVVPTKLEVGDMVTFPIFCQCPTAAQNATALVTYVMQQGDTYASIAAAFAVDAQSLVSLNGPEQGTQLFSEILVPLRRQVPKWLPPIVTRNDASAT

SEQ ID NO: 99 - protein, 274 aa, Lotus japonicus
MKLKTGLLLFFILLLGHVCFHVESNCLKGCDLALASYYILPGVFILQNITTFMQSEIVSSNDAITSYNKDKILNDINIQSFQRLNIPFPCDCIGGEFLGHVFEYSASKGDTYETIANLYYANLTTVDLLKRFNSYDPKNIPVNAKVNVTVNCSCGNSQVSKDYGLFITYPIRPGDTLQDIANQSSLDAGLIQSFNPSVNFSKDSGIAFIPGRYKNGVYVPLYHRTAGLASGAAVGISIAGTFVLLLLAFCMYVRYQKKEEEKAKLPTDISMALS

SEQ ID NO: 100 - protein, 275 aa, Lotus japonicus
MEHPRLGFPITLLLFSFILLPSTSQSKCTHGCALAQASYYLLNGSNLTYISEIMQSSLLTKPEDIVSYNQDTIASKDSVQAGQRINVPFPCDCIEGEFLGHTFQYDVQKGDRYDTIAGTNYANLTTVEWLRRFNSYPPDNIPDTGTLNVTVNCSCGDSGVGDYGLFVTYPLRPGETLGSVASNVKLDSALLQKYNPNVNFNQGSGIVYIPAKDQNGSYVLLGSSSGGLAGGAIAGIAAGVAVCLLLLAGFIYVGYFRKKRIQKEELLSQETRAIF

SEQ ID NO: 101 - protein, 43 aa, Medicago truncatula
PSIQLRNISNFMQSKIVLTNSFDVIMSYNRDVVFDKSGLISYT

SEQ ID NO: 102 - protein, 59 aa, Medicago truncatula
VALASYYIIPSIQLRNISNFMQSKIVLTNSFDVIMSYNRDVVFDKSGLISYTRINVPFP

SEQ ID NO: 103 - protein, 58 aa, Lotus japonicus
LALASYYILPGVFILQNITTFMQSEIVSSNDAITSYNKDKILNDINIQSFQRLNIPFP

SEQ ID NO: 104 - protein, 42 aa, Medicago truncatula; identical to SEQ ID NO: 44

SEQ ID NO: 105 - protein, 60 aa, Medicago truncatula; identical to SEQ ID NO: 49

SEQ ID NO: 106 - protein, 59 aa, Lotus japonicus; identical to SEQ ID NO: 55

SEQ ID NO: 107 - protein, 58 aa, Lotus japonicus
ALAQASYYLLNGSNLTYISEIMQSSLLTKPEDIVSYNQDTIASKDSVQAGQRINVPFP

SEQ ID NO: 108 - skipped

(19) United States
(12) Patent Application Publication
(10) Pub. No.: US 2025/0140337 A1
(43) Pub. Date: May 1, 2025

(54) GENETICALLY ALTERED LYSM RECEPTORS WITH ALTERED AGONIST SPECIFICITY AND AFFINITY

(71) Applicant: Aarhus Universitet, Aarhus (DK)

(72) Inventors: Kasper Røjkjær ANDERSEN, Aarhus (DK); Kira GYSEL, Aarhus (DK); Elena Simona RADUTOIU, Aarhus (DK); Zoltan BOZSOKI, Aarhus (DK); Lene Heegaard MADSEN, Aarhus (DK); Simon Boje HANSEN, Aarhus (DK); Jens Stougaard JENSEN, Aarhus (DK)

(73) Assignee: Aarhus Universitet, Aarhus (DK)

(21) Appl. No.: 18/913,825

(22) Filed: Oct. 11, 2024

Related U.S. Application Data
(62) Division of application No. 17/267,240, filed on Feb. 9, 2021, now Pat. No. 12,190,997.
(60) Provisional application No. 62/718,282, filed on Aug. 13, 2018.

(51) Int. Cl. G16B 15/30; C12N 9/12

(52) U.S. Cl. CPC G16B 15/30; C12N 9/1205

(57) ABSTRACT

Aspects of the present disclosure relates to genetically altered LysM receptors. In particular, the present disclosure relates to a hydrophobic patch into the LysM2 domain which can increase affinity and/or selectivity for LCOs and by replacement of regions in the LysM1 domain with the corresponding regions of the LysM1 domain from a donor LysM receptor that can alter the affinity and/or selectivity for the oligosaccharide particularly for LCOs and can alter the specificity between LCO when using regions from a high affinity and specificity LCO LysM receptor such as a legume NFR1 receptor. The present disclosure also relates to genetically altering LysM receptors in plants to include a hydrophobic patch or alter the hydrophobic patch and to genetically altering LysM receptors in plants by replacement of regions in the LysM2 domain.

### CROSS-REFERENCE TO RELATED APPLICATIONS

This application is a divisional of U.S. application Ser. No. 17/267,240, filed Feb. 9, 2021, which is a national stage application under 35 U.S.C. § 371 of International Application No. PCT/EP2019/071705, filed Aug. 13, 2019, which claims the benefit of U.S. Provisional Application No. 62/718,282, filed Aug. 13, 2018, all of which are hereby incorporated by reference in their entirety.

### INCORPORATION BY REFERENCE OF SEQUENCE LISTING

The present application is being filed along with a Sequence Listing in electronic format. The Sequence Listing is provided as a file entitled 794542000410SubSequenceListing.xml, created Jan. 17, 2025, which is 148,445 bytes in size. The information in the electronic format of the Sequence Listing is incorporated by reference in its entirety.

### TECHNICAL FIELD

The present disclosure relates to genetically altered LysM receptors. In particular, the present disclosure relates to a hydrophobic patch into the LysM2 domain which can increase affinity and/or selectivity for LCOs and by replacement of regions in the LysM1 domain with the corresponding regions of the LysM1 domain from a donor LysM receptor that can alter the affinity and/or selectivity for the oligosaccharide, particularly for LCOs, and can alter the specificity between LCOs when using regions from a high affinity and specificity LCO LysM receptor such as a legume NFR1 receptor. The present disclosure also relates to genetically altering LysM receptors in plants to include a hydrophobic patch or alter the hydrophobic patch and to genetically altering LysM receptors in plants by replacement of regions in the LysM2 domain.

### BACKGROUND

Plants are exposed to a wide variety of microbes in their environment, both benign and pathogenic. To protect against the pathogenic microbes, plants have the ability to recognize specific molecular signals of the microbes through an array of receptors and, depending upon the pattern of the signals, can initiate an appropriate immune response. The molecular signals are derived from secreted materials, cell-wall components, and even cytosolic proteins of the microbes. Chitooligosaccharides (COs) are an important fungal molecular signal that plants recognize through the chitin receptors CEBiP, CERK1, LYK5, and CERK6 (previously called LYS6) found on the plasma membrane. These receptors are in the LysM class of receptors and recognize the size and the acetylation of COs from fungi. Lipo-chitooligosaccharides (LCOs) are another important molecular signal that can be found on both bacteria and fungi that are recognized by other LysM receptors.

In addition to benign and pathogenic microbes, some microbes can be beneficial to plants through association or symbiosis. Plants that enter into symbiotic relationships with certain nitrogen fixing bacteria and fungi need to be able to recognize the specific bacterial or fungal species to initiate the symbiosis while still being able to activate their immune systems to respond to other bacteria and fungi. One important mechanism that allows plants to recognize these specific bacteria or fungi is through specialized LysM receptors that have high affinity, high selectivity, and/or high specificity for the form of LCOs produced by the specific bacteria or fungi while LCOs from other bacteria and fungi are not recognized by these specialized LysM receptors.

Experimental and computational approaches have been used to identify a number of these specialized LysM receptors (also referred to as high affinity and specificity LCO receptors). As these receptors are required for recognizing symbiotic bacterial and fungal species, and for initiating symbiosis, these receptors represent an important component of any plant engineering strategy. Using these receptors, however, will not be particularly straightforward; transferring a specialized LysM receptor into a plant that does not currently have one may require codon optimization, the identification of suitable promoters, the use of targeting signals, and further engineering approaches needed to adapt exogenous sequences for optimal expression. Further, the number of these receptors that have been identified is currently limited.

Moreover, species that already have specialized LysM receptors, e.g., legumes, cannot be easily engineered with new specialized LysM receptors. Currently, legumes are limited to the specific bacterial or fungal species with which they form symbiotic associations. While legumes may have the benefit of existing symbiotic associations, their agricultural potential is limited. For example, legumes cannot currently be easily engineered to have different specificity for different symbiotic microbial species, which would allow legumes to better form associations with the bacterial or fungal species in different soils. Moreover, legumes cannot be easily engineered to have improved specialized LysM receptors. Further, legumes cannot currently be engineered to have synergistic symbiotic requirements with other crops grown in rotation with them. Editing approaches are needed for both the modification of endogenous LysM receptors into specialized LysM receptors able to perceive symbiotic bacterial and fungal species, and the modification of specialized LysM receptors into specialized LysM receptors with different specific recognition of symbiotic bacterial and fungal species.

### BRIEF SUMMARY

In order to meet these needs, the present disclosure provides complementary means of modifying LysM receptors by introduction of a hydrophobic patch into the LysM2 domain which can increase affinity and/or selectivity for LCOs, and by replacement of regions in the LysM1 domain with the corresponding regions of the LysM1 domain from a donor LysM receptor that can alter the affinity and/or selectivity for the oligosaccharide, particularly for LCOs, and can alter the specificity between LCOs when using regions from a high affinity and specificity LCO LysM receptor such as a legume NFR1 receptor.

Certain aspects of the present disclosure relate to a modified plant LysM receptor comprising a LysM2 domain modified to comprise a hydrophobic patch on the surface of the LysM2 domain. In some embodiments, the modified LysM2 domain binds a lipo-chitooligosaccharide (LCO). In some embodiments, the modified LysM2 domain binds the LCO with higher affinity than the unmodified LysM2 domain. In some embodiments, the modified LysM2 domain binds the LCO with higher selectivity for the LCO than the unmodified LysM2 domain. In some embodiments, the higher affinity or higher selectivity is due to the hydrophobic patch interacting with the LCO. In some embodiments, the higher affinity or higher selectivity is due to the hydrophobic patch interacting with the lipid of the LCO. In some embodiments, the LCO is produced by nitrogen-fixing bacteria or by mycorrhizal fungi. In some embodiments, the LCO is produced by nitrogen-fixing bacteria selected from the group of Mesorhizobium loti, Mesorhizobium huakuii, Mesorhizobium mediterraneum, Mesorhizobium ciceri, Mesorhizobium spp., Rhizobium mongolense, Rhizobium tropici, Rhizobium etli phaseoli, Rhizobium giardinii, Rhizobium leguminosarum optionally R. leguminosarum trifolii, R. leguminosarum viciae, and R. leguminosarum phaseoli, Burkholderiales optionally symbionts of Mimosa, Sinorhizobium meliloti, Sinorhizobium medicae, Sinorhizobium fredii, Sinorhizobium NGR234, Azorhizobium caulinodans, Bradyrhizobium japonicum, Bradyrhizobium elkanii, Bradyrhizobium liaonginense, Frankia spp., and any combination thereof, or by mycorrhizal fungi selected from the group consisting of Acaulosporaceae spp., Diversisporaceae spp., Gigasporaceae spp., Pacisporaceae spp., Funneliformis spp., Glomus spp., Rhizophagus spp., Sclerocystis spp., Septoglomus spp., Claroideoglomus spp., Ambispora spp., Archaeospora spp., Geosiphon pyriformis, Paraglomus spp., other species in the division Glomeromycota, or any combination thereof.

In some embodiments of any of the above embodiments, the LysM receptor is selected from the group consisting of a LysM chitooligosaccharide (CO) receptor, a LysM LCO receptor, and a LysM peptidoglycan (PGN) receptor. In some embodiments, the hydrophobic patch is adjacent to a chitin binding motif if the LysM receptor is the LysM CO receptor or the LysM LCO receptor. In some embodiments, the hydrophobic patch is within 30 Å, 20 Å, 10 Å, 7.5 Å, 5 Å, 4 Å, 3 Å, 2 Å, 1.5 Å, or 1 Å of a chitin binding motif if the LysM receptor is the LysM CO receptor or the LysM LCO receptor. In some embodiments, the hydrophobic patch is adjacent to a glycan binding motif if the LysM receptor is the LysM PGN receptor. In some embodiments, the hydrophobic patch is within 30 Å, 20 Å, 10 Å, 7.5 Å, 5 Å, 4 Å, 3 Å, 2 Å, 1.5 Å, or 1 Å of a glycan binding motif if the LysM receptor is the LysM PGN receptor. In some embodiments, the LysM receptor is not an exopolysaccharide (EPS) receptor.

In some embodiments of any of the above embodiments, the LysM receptor is a polypeptide having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO:34 (i.e., Lotus CERK6; BAI79273.1_LjCERK6). In some embodiments of any of the above embodiments, the LysM receptor is a polypeptide having amino acid sequence SEQ ID NO:34 (i.e., Lotus CERK6; BAI79273.1_LjCERK6).

In some embodiments of any of the above embodiments, the hydrophobic patch was generated by deleting at least one non-hydrophobic amino acid residue, substituting at least one amino acid residue with a more hydrophobic amino acid, or combinations thereof. In some embodiments, the at least one amino acid was identified by an amino acid sequence alignment with a LysM2 domain from a LysM high affinity LCO receptor that naturally has a hydrophobic patch that interacts with LCO. In some embodiments, the at least one amino acid corresponds to an amino acid that is in bold in FIGS. 12A-12E and FIG. 13C. In some embodiments, the at least one amino acid corresponds to an amino acid that is in bold in FIGS. 12A-12E and FIG. 13C or corresponds to an amino acid that is immediately N-terminal or C-terminal to an amino acid that is in bold in FIGS. 12A-12E and FIG. 13C. In some embodiments, the at least one amino acid corresponds to an amino acid that is in bold in FIGS. 12A-12E in a known LCO receptor. In some embodiments, the at least one amino acid corresponds to an amino acid that is in bold in FIGS. 12A-12E in a known LCO receptor or corresponds to an amino acid that is immediately N-terminal or C-terminal to an amino acid that is in bold in FIGS. 12A-12E in a known LCO receptor. In some embodiments, the at least one amino acid was identified by structural modelling to identify a region in LysM2 where the hydrophobic patch can be engineered. In some embodiments, the structural modeling used the unmodified plant LysM amino acid sequence and a LysM domain three dimensional structure that has a known hydrophobic patch. In some embodiments, the LysM domain three dimensional structure is a Medicago NFP ectodomain. In some embodiments, the known hydrophobic patch amino acid residues of the LysM domain three dimensional structure are or correspond to L147, L151, L152, L154, T156, K157 and V158 of the Medicago NFP ectodomain. In some embodiments, the alpha carbon of at least one amino acid was within 3 Å of an alpha carbon of a known hydrophobic patch amino acid residue in the structural alignment. In some embodiments, the structural modeling was performed using SWISS-MODEL, PDB2PQR, APBS, PyMol, and APBS tools 2.1.

In some aspects, the present disclosure relates to a modified plant LysM receptor comprising a first LysM1 domain modified to replace at least part of the first LysM1 domain with at least part of a second LysM1 domain. In some embodiments, the first LysM1 domain is modified by substituting a first part of the first LysM1 domain with a third part of a second LysM1 domain and/or by substituting a second part of the first LysM1 domain with a fourth part of the second LysM1 domain. In some embodiments, the first LysM1 domain and the second LysM1 domain have different affinities, selectivities, and/or specificities for oligosaccharides and the modification of the first LysM1 domain alters the affinity, selectivity, and/or specificity to be more like the second LysM1 domain. In some embodiments, the first part and the third part correspond to SEQ ID NO:30 [Lotus CERK6 region II 43-53] or NGSNLTYISEI, SEQ ID NO: 28 [Lotus NFR 1 region II 41-52] or PGVFILQNITTF; and wherein the second part and the fourth part correspond to SEQ ID NO:31 [Lotus CERK6 region IV 74-82] or ASKDSVQAG; SEQ ID NO:29 [Lotus NFR1 region IV 73-81], or LNDINIQSF. In some embodiments, the first LysM1 domain is selected from the group of SEQ ID NO:32 [LysM1 domain Lotus NFR1; LjNFR1/26-95], SEQ ID NO:33 [LysM1 domain Medicago LYK3; MtLYK3/25-95], or NFR1 DLALASYYILPGVFILQNITTFMQSEIVSSNDAITSYNKDKILNDINIQSFQRLNIPFP (SEQ ID NO: 55); and the second LysM1 domain is CERK6: ALAQASYYLLNGSNLTYISEIMQSSLLTKPEDIVSYNQDTIASKDSVQAGQRINVPFP (SEQ ID NO:107). In some embodiments, the first part is selected from SEQ ID NO:30 [Lotus CERK6 region II 43-53] or NGSNLTYISEI; the second part is selected from SEQ ID NO: 31 [Lotus CERK6 region IV 74-82] or ASKDSVQAG; the third part is selected from SEQ ID NO: 28 [Lotus NFR1 region II 41-52] or PGVFILQNITTF; and the fourth part is selected from SEQ ID NO: 29 [Lotus NFR1 region IV 73-81] or LNDINIQSF. In some embodiments, the entire first LysM1 domain was replaced with the entire second LysM1 domain. In some embodiments, the modified LysM1 domain binds a lipo-chitooligosaccharide (LCO) produced by nitrogen-fixing bacteria or by mycorrhizal fungi. In some embodiments, the LCO is produced by nitrogen-fixing bacteria selected from the group consisting of Mesorhizobium loti, Mesorhizobium huakuii, Mesorhizobium mediterraneum, Mesorhizobium ciceri, Mesorhizobium spp., Rhizobium mongolense, Rhizobium tropici, Rhizobium etli phaseoli, Rhizobium giardinii, Rhizobium leguminosarum optionally R. leguminosarum trifolii, R. leguminosarum viciae, and R. leguminosarum phaseoli, Burkholderiales optionally symbionts of Mimosa, Sinorhizobium caulinodans, Bradyrhizobium japonicum, Bradyrhizobium elkanii, Bradyrhizobium liaonginense, Frankia spp., and any combination thereof, or by mycorrhizal fungi selected from the group consisting of Acaulosporaceae spp., Diversisporaceae spp., Gigasporaceae spp., Pacisporaceae spp., Funneliformis spp., Glomus spp., Rhizophagus spp., Sclerocystis spp., Septoglomus spp., Claroideoglomus spp., Ambispora spp., Archaeospora spp., Geosiphon pyriformis, Paraglomus spp., other species in the division Glomeromycota, and any combination thereof. In some embodiments, the modified LysM1 domain binds an LCO with higher affinity than an unmodified LysM1 domain. In some embodiments, the modified LysM1 domain binds LCOs with higher selectivity than an unmodified LysM1 domain. In some embodiments, the modified LysM1 domain binds LCOs with altered specificity as compared to an unmodified LysM1 domain. In some embodiments, structural modelling was used to define the LysM1 domain and was used to identify the first part, the second part, the third part, and/or the fourth part for substitution. In some embodiments, the receptor of the above embodiments further contains a LysM2 domain modified to contain a hydrophobic patch as in any one of the previous embodiments relating to modifying the LysM2 domain.

In some aspects, the present disclosure relates to a genetically altered plant or part thereof, comprising a nucleic acid sequence encoding the modified plant LysM receptor of any one of the preceding embodiments. In some embodiments, the modified plant LysM receptor has higher affinity, higher selectivity, and/or altered specificity for LCOs than the unmodified plant LysM receptor and the expression of the modified plant LysM receptor allows the plant or part thereof to recognize LCOs with high affinity, high selectivity, and/or altered specificity. In some embodiments, the LCOs are produced by nitrogen-fixing bacteria or by mycorrhizal fungi. In some embodiments, the LCOs are produced by nitrogen-fixing bacteria selected from the group consisting of Mesorhizobium loti, Mesorhizobium huakuii, Mesorhizobium mediterraneum, Mesorhizobium ciceri, Mesorhizobium spp., Rhizobium mongolense, Rhizobium tropici, Rhizobium etli phaseoli, Rhizobium giardinii, Rhizobium leguminosarum optionally R. leguminosarum trifolii, R. leguminosarum viciae, and R. leguminosarum phaseoli, Burkholderiales optionally symbionts of Mimosa, Sinorhizobium meliloti, Sinorhizobium medicae, Sinorhizobium fredii, Sinorhizobium NGR234, Azorhizobium caulinodans, Bradyrhizobium japonicum, Bradyrhizobium elkanii, Bradyrhizobium liaonginense, Frankia spp., and any combination thereof, or by or by mycorrhizal fungi selected from the group consisting of Acaulosporaceae spp., Diversisporaceae spp., Gigasporaceae spp., Pacisporaceae spp., Funneliformis spp., Glomus spp., Rhizophagus spp., Sclerocystis spp., Septoglomus spp., Claroideoglomus spp., Ambispora spp., Archaeospora spp., Geosiphon pyriformis, Paraglomus spp., other species in the division Glomeromycota, and any combination thereof. In some embodiments, the modified polypeptide is localized to a plant cell plasma membrane. In some embodiments, the plant cell is a root cell. In some embodiments, the root cell is a root epidermal cell. In some embodiments, the modified polypeptide is expressed in a developing plant root system. In some embodiments, the nucleic acid sequence is operably linked to a promoter. In some embodiments, the promoter is a root specific promoter. In some embodiments, the promoter is selected from the group of a NFR1/LYK3/CERK6 or NFR5/NFP promoter, the Lotus NFR5 promoter (SEQ ID NO:24) and the Lotus NFR1 promoters (SEQ ID NO:25) the maize allothioneine promoter, the chitinase promoter, the maize ZRP2 promoter, the tomato LeExtl promoter, the glutamine synthetase soybean root promoter, the RCC3 promoter, the rice antiquitine promoter, the LRR receptor kinase promoter, or the Arabidopsis pCO2 promoter. In some embodiments, the promoter is a constitutive promoter optionally selected from the group of the CaMV35S promoter, a derivative of the CaMV35S promoter, the maize ubiquitin promoter, the trefoil promoter, a vein mosaic cassava virus promoter, or the Arabidopsis UBQ10 promoter. In some embodiments, the plant is selected from the group of corn, rice, barley, wheat, Trema spp., apple, pear, plum, apricot, peach, almond, walnut, strawberry, raspberry, blackberry, red currant, black currant, melon, cucumber, pumpkin, squash, grape, bean, pea, chickpea, cowpca, pigeon pea, lentil, Bambara groundnut, lupin, pulses, Medicago spp., Lotus spp., forage legumes, indigo, legume trees, or hemp. In some embodiments, the plant part is a leaf, a stem, a root, a root primordia, a flower, a seed, a fruit, a kernel, a grain, a cell, or a portion thereof.

In some aspects, the present disclosure relates to a genetically altered plant or part thereof, comprising a first nucleic acid sequence encoding a modified plant LysM receptor where the LysM1 domain has been modified as in any of the preceding embodiments relating to modification to the LysM1 domain and a second nucleic acid sequence encoding a modified plant LysM receptor where the LysM2 domain has been modified to include a hydrophobic patch as in any of the preceding embodiments relating to modifications to the LysM2 domain. In some embodiments, the modified plant LysM receptor has higher affinity, higher selectivity, and/or altered specificity for LCOs than the unmodified plant LysM receptor and the expression of the modified plant LysM receptor allows the plant or part thereof to recognize LCOs with high affinity, high selectivity, and/or altered specificity. In some embodiments, the LCOs are produced by nitrogen-fixing bacteria or by mycorrhizal fungi. In some embodiments, the LCOs are produced by nitrogen-fixing bacteria selected from the group consisting of Mesorhizobium loti, Mesorhizobium huakuii, Mesorhizobium mediterraneum, Mesorhizobium ciceri, Mesorhizobium spp., Rhizobium mongolense, Rhizobium tropici, Rhizobium etli phaseoli, Rhizobium giardinii, Rhizobium leguminosarum optionally R. leguminosarum trifolii, R. leguminosarum viciae, and R. leguminosarum phaseoli, Burkholderiales optionally symbionts of Mimosa, Sinorhizobium meliloti, Sinorhizobium medicae, Sinorhizobium fredii, Sinorhizobium NGR234, Azorhizobium caulinodans, Bradyrhizobium japonicum, Bradyrhizobium elkanii, Bradyrhizobium liaonginense, Frankia spp., and any combination thereof, or by or by mycorrhizal fungi selected from the group consisting of Acaulosporaceae spp., Diversisporaceae spp., Gigasporaceae spp., Pacisporaceae spp., Funneliformis spp., Glomus spp., Rhizophagus spp., Sclerocystis spp., Septoglomus spp., Claroideoglomus spp., Ambispora spp., Archaeospora spp., Geosiphon pyriformis, Paraglomus spp., other species in the division Glomeromycota, and any combination thereof. In some embodiments, the modified polypeptide is localized to a plant cell plasma membrane. In some embodiments, the plant cell is a root cell. In some embodiments, the root cell is a root epidermal cell. In some embodiments, the modified polypeptide is expressed in a developing plant root system. In some embodiments, the first nucleic acid or second nucleic acid sequence is operably linked to a promoter. In some embodiments, the promoter is a root specific promoter. In some embodiments, the promoter is selected from the group of a NFR1/LYK3/CERK6 or NFR5/NFP promoter, the Lotus NFR5 promoter (SEQ ID NO: 24) and the Lotus NFR1 promoters (SEQ ID NO:25) the maize allothioneine promoter, the chitinase promoter, the maize ZRP2 promoter, the tomato LeExtl promoter, the glutamine synthetase soybean root promoter, the RCC3 promoter, the rice antiquitine promoter, the LRR receptor kinase promoter, or the Arabidopsis pCO2 promoter. In some embodiments, the promoter is a constitutive promoter optionally selected from the group of the CaMV35S promoter, a derivative of the CaMV35S promoter, the maize ubiquitin promoter, the trefoil promoter, a vein mosaic cassava virus promoter, or the Arabidopsis UBQ10 promoter. In some embodiments, the plant is selected from the group of corn, rice, barley, wheat, Trema spp., apple, pear, plum, apricot, peach, almond, walnut, strawberry, raspberry, blackberry, red currant, black currant, melon, cucumber, pumpkin, squash, grape, bean, pea, chickpea, cowpea, pigeon pea, lentil, Bambara groundnut, lupin, pulses, Medicago spp., Lotus spp., forage legumes, indigo, legume trees, or hemp. In some embodiments, the plant part is a leaf, a stem, a root, a root primordia, a flower, a seed, a fruit, a kernel, a grain, a cell, or a portion thereof. In some embodiments, the plant part is a fruit, a kernel, or a grain.

In some aspects, the present disclosure relates to a pollen grain or an ovule of a genetically altered plant of any of the above embodiments relating to plants.

In some aspects, the present disclosure relates to a protoplast from a genetically altered plant of any of the above embodiments relating to plants.

In some aspects, the present disclosure relates to a tissue culture produced from protoplasts or cells from a genetically altered plant of any of the above embodiments relating to plants, wherein the cells or protoplasts are produced from a plant part selected from the group consisting of leaf, anther, pistil, stem, petiole, root, root primordia, root tip, fruit, seed, flower, cotyledon, hypocotyl, embryo, and meristematic cell.

In some aspects, the present disclosure relates to a method of producing the genetically altered plant of any one of the above embodiments relating to plants, comprising introducing a genetic alteration to the plant comprising the nucleic acid sequence. In some embodiments, the nucleic acid sequence, the first nucleic acid sequence, and/or the second nucleic acid sequence is operably linked to a promoter. In some embodiments, the promoter is a root specific promoter. In some embodiments, the promoter is selected from the group of a NFR1/LYK3/CERK6 or NFR5/NFP promoter, the Lotus NFR5 promoter (SEQ ID NO: 24) and the Lotus NFR 1 promoters (SEQ ID NO:25) the maize allothioneine promoter, the chitinase promoter, the maize ZRP2 promoter, the tomato LeExtl promoter, the glutamine synthetase soybean root promoter, the RCC3 promoter, the rice antiquitine promoter, the LRR receptor kinase promoter, or the Arabidopsis pCO2 promoter. In some embodiments, the promoter is a constitutive promoter optionally selected from the group of the CaMV35S promoter (KAY et al. Science, 236, 4805, 1987), a derivative of the CaMV35S promoter, the maize ubiquitin promoter, the trefoil promoter, a vein mosaic cassava virus promoter, or the Arabidopsis UBQ10 promoter. In some embodiments, the nucleic acid sequence, the first nucleic acid sequence, and/or the second nucleic acid sequence is inserted into the genome of the plant so that the nucleic acid sequence, the first nucleic acid sequence, and/or the second nucleic acid sequence is operably linked to an endogenous promoter. In some embodiments, the endogenous promoter is a root specific promoter.

Additional aspects of the present disclosure relate to a modified plant LysM receptor including a LysM2 domain modified to include a hydrophobic patch on the surface of the LysM2 domain. In some embodiments, the modified LysM2 domain binds a lipo-chitooligosaccharide (LCO). In some embodiments, the modified LysM2 domain binds the LCO with higher affinity than the unmodified LysM2 domain. In some embodiments, the modified LysM2 domain binds the LCO with higher selectivity for the LCO than the unmodified LysM2 domain. In some embodiments, the higher affinity or higher selectivity is due to the hydrophobic patch interacting with the LCO. In some embodiments, the higher affinity or higher selectivity is due to the hydrophobic patch interacting with the lipid of the LCO. In some embodiments, the LCO is produced by nitrogen-fixing bacteria or by mycorrhizal fungi. In some embodiments, the LCO is produced by nitrogen-fixing bacteria selected from the group of Mesorhizobium loti, Mesorhizobium huakuii, Mesorhizobium mediterraneum, Mesorhizobium ciceri, Mesorhizobium spp., Rhizobium mongolense, Rhizobium tropici, Rhizobium etli phaseoli, Rhizobium giardinii, Rhizobium leguminosarum optionally R. leguminosarum trifolii, R. leguminosarum viciae, and R. leguminosarum phaseoli, Burkholderiales optionally symbionts of Mimosa, Sinorhizobium meliloti, Sinorhizobium medicae, Sinorhizobium fredii, Sinorhizobium NGR234, Azorhizobium caulinodans, Bradyrhizobium japonicum, Bradyrhizobium elkanii, Bradyrhizobium liaonginense, Frankia spp., or any combination thereof, or by mycorrhizal fungi selected from the group of Acaulosporaceae spp., Diversisporaceae spp., Gigasporaceae spp., Pacisporaceae spp., Funneliformis spp., Glomus spp., Rhizophagus spp., Sclerocystis spp., Septoglomus spp., Claroideoglomus spp., Ambispora spp., Archaeospora spp., Geosiphon pyriformis, Paraglomus spp., other species in the division Glomeromycota, or any combination thereof.

In some embodiments of any of the above embodiments, the LysM receptor is selected from the group of a LysM chitooligosaccharide (CO) receptor, a LysM LCO receptor, or a LysM peptidoglycan (PGN) receptor. In some embodiments, the hydrophobic patch is adjacent to a chitin binding motif if the LysM receptor is the LysM CO receptor or the LysM LCO receptor. In some embodiments, the hydrophobic patch is within 30 Å, 20 Å, 10 Å, 7.5 Å, 5 Å, 4 Å, 3 Å, 2 Å, 1.5 Å, or 1 Å of a chitin binding motif if the LysM receptor is the LysM CO receptor or the LysM LCO receptor. In some embodiments, the hydrophobic patch is adjacent to a glycan binding motif if the LysM receptor is the LysM PGN receptor. In some embodiments, the hydrophobic patch is within 30 Å, 20 Å, 10 Å, 7.5 Å, 5 Å, 4 Å, 3 Å, 2 Å, 1.5 Å, or 1 Å of a glycan binding motif if the LysM receptor is the LysM PGN receptor. In some embodiments, the LysM receptor is not an exopolysaccharide (EPS) receptor.

In some embodiments of any of the above embodiments, the LysM receptor is a polypeptide having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO:34 (i.e., Lotus CERK6; BAI79273.1_LjCERK6). In some embodiments of any of the above embodiments, the LysM receptor is a polypeptide having amino acid sequence SEQ ID NO:34 (i.e., Lotus CERK6; BAI79273.1_LjCERK6).

In some embodiments of any of the above embodiments, the hydrophobic patch was generated by deleting at least one non-hydrophobic amino acid residue, substituting at least one amino acid residue with a more hydrophobic amino acid, or combinations thereof. In some embodiments of any of the above embodiments, the hydrophobic patch was generated by modifying an existing hydrophobic patch in the unmodified LysM receptor. In some embodiments, the unmodified LysM receptor was modified by deleting at least one non-hydrophobic amino acid residue, substituting at least one amino acid residue with a more hydrophobic amino acid, substituting at least one hydrophobic amino acid residue with another hydrophobic amino acid residue, or combinations thereof. In some embodiments, the at least one amino acid was identified by an amino acid sequence alignment with a LysM2 domain from a LysM high affinity LCO receptor that naturally has a hydrophobic patch that interacts with LCO. In some embodiments, the at least one amino acid corresponds to an amino acid that is in bold underline in FIGS. 12A-12E and FIG. 13C. In some embodiments, the at least one amino acid corresponds to an amino acid that is in bold underline in FIGS. 12A-12E and FIG. 13C or corresponds to an amino acid that is immediately N-terminal or C-terminal to an amino acid that is in bold underline in FIGS. 12A-12E and FIG. 13C. In some embodiments, the at least one amino acid corresponds to an amino acid that is in bold underline in FIGS. 12A-12E in a known LCO receptor. In some embodiments, the at least one amino acid corresponds to an amino acid that is in bold underline in FIGS. 12A-12E in a known LCO receptor or corresponds to an amino acid that is immediately N-terminal or C-terminal to an amino acid that is in bold underline in FIGS. 12A-12E in a known LCO receptor. In some embodiments, the at least one amino acid was identified by structural modelling to identify a region in LysM2 where the hydrophobic patch can be engineered. In some embodiments, the structural modeling used the unmodified plant LysM amino acid sequence and a LysM domain three dimensional structure that has a known hydrophobic patch. In some embodiments, the LysM domain three dimensional structure is a Medicago NFP ectodomain. In some embodiments, the known hydrophobic patch amino acid residues of the LysM domain three dimensional structure are or correspond to L147, L151, L152, L154, T156, K157 and V158 of the Medicago NFP ectodomain. In some embodiments, the LysM domain three dimensional structure is a Lotus LYS11 ectodomain. In some embodiments, the unmodified LysM receptor is the Lotus LYS11 receptor and the existing hydrophobic patch amino acid residues of the LysM domain that are modified are or correspond to K100, E101, G102, E103, S104, Y105, Y106, N128, and Y129 of the Lotus LYS11 ectodomain. In some embodiments, the alpha carbon of at least one amino acid was within 3 Å of an alpha carbon of a known hydrophobic patch amino acid residue in the structural alignment. In some embodiments, the structural modeling was performed using SWISS-MODEL, PDB2PQR, APBS, PyMol, and APBS tools 2.1. In some embodiments of any of the above embodiments, cither or both (i) 80% or fewer, 70% or fewer, 60% or fewer, 50% or fewer, 40% or fewer, 30% or fewer, or 20% or fewer of amino acid residues in the LysM2 domain of the unmodified LysM receptor were substituted or deleted to generate the modified plant LysM receptor, and (ii) the entire LysM2 domain in the unmodified plant LysM receptor was not substituted with another entire LysM2 domain to generate the modified plant LysM receptor. In some embodiments of any of the above embodiments, the unmodified plant LysM receptor was selected using the method of any one of the aspects of the present disclosure relating to such selection including any and all embodiments thereof.

In some aspects, the present disclosure relates to a modified plant LysM receptor including a first LysM1 domain modified to replace at least part of the first LysM1 domain with at least part of a second LysM1 domain. In some embodiments, the first LysM1 domain is modified by substituting a first part of the first LysM1 domain with a third part of a second LysM1 domain and/or by substituting a second part of the first LysM1 domain with a fourth part of the second LysM1 domain. In some embodiments, the first LysM1 domain and the second LysM1 domain have different affinities, selectivities, and/or specificities for oligosaccharides and the modification of the first LysM1 domain alters the affinity, selectivity, and/or specificity to be more like the second LysM1 domain. In some embodiments, the first part and the third part correspond to SEQ ID NO:30 [Lotus CERK6 region II 43-53] or NGSNLTYISEI, SEQ ID NO: 28 [Lotus NFR1 region II 41-52] or PGVFILQNITTF; and wherein the second part and the fourth part correspond to SEQ ID NO: 31 [Lotus CERK6 region IV 74-82] or ASKDSVQAG; SEQ ID NO:29 [Lotus NFR1 region IV 73-81], or LNDINIQSF. In some embodiments, the first LysM1 domain is selected from the group of SEQ ID NO:32 [LysM1 domain Lotus NFR1; LjNFR1/26-95], SEQ ID NO:33 [LysM1 domain Medicago LYK3; MtLYK3/25-95], or NFR1 DLALASYYILPGVFILQNITTFMQSEIVSSNDAITSYNKDKILNDINIQSFORLNIPFP (SEQ ID NO: 55); and the second LysM1 domain is CERK6: ALAQASYYLLNGSNLTYISEIMQSSLLTKPEDIVSYNQDTIASKDSVQAGQRINVPFP (SEQ ID NO:107). In some embodiments, the first part is selected from SEQ ID NO:30 [Lotus CERK6 region II 43-53] or NGSNLTYISEI; the second part is selected from SEQ ID NO: 31 [Lotus CERK6 region IV 74-82] or ASKDSVQAG; the third part is selected from SEQ ID NO: 28 [Lotus NFR 1 region II 41-52] or PGVFILQNITTF; and the fourth part is selected from SEQ ID NO: 29 [Lotus NFR 1 region IV 73-81] or LNDINIQSF. In some embodiments of any of the above embodiments including the first LysM1 domain being modified to replace at least part of the first LysM1 domain with at least part of a second LysM1 domain, the first LysM1 domain is further modified by substituting a fifth part of the first LysM1 domain with a sixth part of a second LysM1 domain. In some embodiments, the first LysM1 domain is SEQ ID NO:115 [LysM1 domain Lotus NFR1; LjNFR1/32-89] or SEQ ID NO: 106 [LysM1 domain Lotus NFR1; LjNFR1/31-89] and the second LysM1 domain is SEQ ID NO:114 [LysM1 domain Medicago LYK3; MtLYK3/31-89] or SEQ ID NO: 105 [LysM1 domain Medicago LYK3; MtLYK3/30-89]. In some embodiments, wherein the fifth part is SEQ ID NO:53 [Lotus NFR1 region III 59-62; LjNFR1/56-92], and wherein the sixth part is SEQ ID NO:46 [Medicago LYK3 region III 57-62; MtLYK3/57-62]. In some embodiments, the first LysM1 domain is modified by substituting a seventh part of the first LysM1 domain, wherein the seventh part spans the first part of the first LysM1 domain, the second part of the first LysM1 domain, and the fifth part of the first LysM1 domain, with an eighth part of the second LysM1 domain, wherein the eighth part spans the third part of the second LysM1 domain, the fourth part of the second LysM1 domain, and the sixth part of the second LysM1 domain. In some embodiments, the seventh part of the first LysM1 domain is SEQ ID NO:51 [Lotus NFR1 regions II-IV 41-82; LjNFR1/41-82], and the eighth part of the second LysM1 domain is SEQ ID NO:113 [Medicago LYK3 regions II-IV 40-82; MtLYK3/40-82] or SEQ ID NO: 104 [Medicago LYK3 regions II-IV 41-82; MtLYK3/41-82]. In some embodiments, the first LysM1 domain is SEQ ID NO:33 [LysM1 domain Medicago LYK3; MtLYK3/31-89] and the second LysM1 domain is SEQ ID NO:32 [LysM1 domain Lotus NFR1; LjNFR1/32-89]. In some embodiments, the fifth part is SEQ ID NO:46 [Medicago LYK3 region III 57-62; MtLYK3/57-62], and the sixth part is SEQ ID NO:53 [Lotus NFR1 region III 59-62; LjNFR1/59-62]. In some embodiments of any of the above embodiments including the first LysM1 domain being SEQ ID NO:33 and the second LysM1 domain being SEQ ID NO:32, the first LysM1 domain is modified by substituting a seventh part of the first LysM1 domain, wherein the seventh part spans the first part of the first LysM1 domain, the second part of the first LysM1 domain, and the fifth part of the first LysM1 domain, with an eighth part of the second LysM1 domain, wherein the eighth part spans the third part of the second LysM1 domain, the fourth part of the second LysM1 domain, and the sixth part of the second LysM1 domain. In some embodiments, the seventh part of the first LysM1 domain is SEQ ID NO:51 [Lotus NFR1 regions II-IV 41-82; LjNFR1/41-82], and the eighth part of the second LysM1 domain is SEQ ID NO:113 [Medicago LYK3 regions II-IV 40-82; MtLYK3/40-82] or SEQ ID NO: 104 [Medicago LYK3 regions II-IV 41-82; MtLYK3/41-82].

In some embodiments of any of the above embodiments including the first LysM1 domain being modified to replace at least part of the first LysM1 domain with at least part of a second LysM1 domain, the entire first LysM1 domain was replaced with the entire second LysM1 domain. In some embodiments of any of the above embodiments including the first LysM1 domain being modified to replace at least part of the first LysM1 domain with at least part of a second LysM1 domain, either or both (i) 80% or fewer, 70% or fewer, 60% or fewer, 50% or fewer, 40% or fewer, 30% or fewer, or 20% or fewer of amino acid residues in the first LysM1 domain were substituted or deleted with the corresponding amino acid residues of the second LysM1 domain, and (ii) the entire LysM1 domain in the unmodified plant LysM receptor was not substituted with another entire LysM2 domain to generate the modified plant LysM receptor. In some embodiments, the modified LysM1 domain binds a lipo-chitooligosaccharide (LCO) produced by nitrogen-fixing bacteria or by mycorrhizal fungi. In some embodiments, the LCO is produced by nitrogen-fixing bacteria selected from the group of Mesorhizobium loti, Mesorhizobium huakuii, Mesorhizobium mediterraneum, Mesorhizobium ciceri, Mesorhizobium spp., Rhizobium mongolense, Rhizobium tropici, Rhizobium etli phaseoli, Rhizobium giardinii, Rhizobium leguminosarum optionally R. leguminosarum trifolii, R. leguminosarum viciae, and R. leguminosarum phaseoli, Burkholderiales optionally symbionts of Mimosa, Sinorhizobium meliloti, Sinorhizobium medicae, Sinorhizobium fredii, Sinorhizobium NGR234, Azorhizobium caulinodans, Bradyrhizobium japonicum, Bradyrhizobium elkanii, Bradyrhizobium liaonginense, Frankia spp., or any combination thereof, or by mycorrhizal fungi selected from the group of Acaulosporaceae spp., Diversisporaceae spp., Gigasporaceae spp., Pacisporaceae spp., Funneliformis spp., Glomus spp., Rhizophagus spp., Sclerocystis spp., Septoglomus spp., Claroideoglomus spp., Ambispora spp., Archaeospora spp., Geosiphon pyriformis, Paraglomus spp., other species in the division Glomeromycota, or any combination thereof. In some embodiments, the modified LysM1 domain binds an LCO with higher affinity than an unmodified LysM1 domain. In some embodiments, the modified LysM1 domain binds LCOs with higher selectivity than an unmodified LysM1 domain. In some embodiments, the modified LysM1 domain binds LCOs with altered specificity as compared to an unmodified LysM1 domain. In some embodiments, structural modelling was used to define the LysM1 domain and was used to identify the first part, the second part, the third part, and/or the fourth part for substitution. In some embodiments, the unmodified plant LysM receptor was selected using the method of any one of the aspects of the present disclosure relating to such selection including any and all embodiments thereof and the second LysM2 domain is from the donor plant LysM receptor. In some embodiments, the receptor of the above embodiments further contains a LysM2 domain modified to contain a hydrophobic patch as in any one of the previous embodiments relating to modifying the LysM2 domain.

In some aspects, the present disclosure relates to a genetically altered plant or part thereof, including a nucleic acid sequence encoding the modified plant LysM receptor of any one of the preceding embodiments. In some embodiments, the modified plant LysM receptor has higher affinity, higher selectivity, and/or altered specificity for LCOs than the unmodified plant LysM receptor and the expression of the modified plant LysM receptor allows the plant or part thereof to recognize LCOs with high affinity, high selectivity, and/or altered specificity. In some embodiments, the LCOs are produced by nitrogen-fixing bacteria or by mycorrhizal fungi. In some embodiments, the LCOs are produced by nitrogen-fixing bacteria selected from the group of Mesorhizobium loti, Mesorhizobium huakuii, Mesorhizobium mediterraneum, Mesorhizobium ciceri, Mesorhizobium spp., Rhizobium mongolense, Rhizobium tropici, Rhizobium etli phaseoli, Rhizobium giardinii, Rhizobium leguminosarum optionally R. leguminosarum trifolii, R. leguminosarum viciae, and R. leguminosarum phaseoli, Burkholderiales optionally symbionts of Mimosa, Sinorhizobium meliloti, Sinorhizobium medicae, Sinorhizobium fredii, Sinorhizobium NGR234, Azorhizobium caulinodans, Bradyrhizobium japonicum, Bradyrhizobium elkanii, Bradyrhizobium liaonginense, Frankia spp., or any combination thereof, or by or by mycorrhizal fungi selected from the group of Acaulosporaceae spp., Diversisporaceae spp., Gigasporaceae spp., Pacisporaceae spp., Funneliformis spp., Glomus spp., Rhizophagus spp., Sclerocystis spp., Septoglomus spp., Claroideoglomus spp., Ambispora spp., Archaeospora spp., Geosiphon pyriformis, Paraglomus spp., other species in the division Glomeromycota, or any combination thereof. In some embodiments, the modified polypeptide is localized to a plant cell plasma membrane. In some embodiments, the plant cell is a root cell. In some embodiments, the root cell is a root epidermal cell. In some embodiments, the modified polypeptide is expressed in a developing plant root system. In some embodiments, the nucleic acid sequence is operably linked to a promoter. In some embodiments, the promoter is a root specific promoter. In some embodiments, the promoter is selected from the group of a NFR1/LYK3/CERK6 or NFR5/NFP promoter, the Lotus NFR5 promoter (SEQ ID NO:24) and the Lotus NFR1 promoters (SEQ ID NO: 25) the maize allothioneine promoter, the chitinase promoter, the maize ZRP2 promoter, the tomato LeExtl promoter, the glutamine synthetase soybean root promoter, the RCC3 promoter, the rice antiquitine promoter, the LRR receptor kinase promoter, or the Arabidopsis pCO2 promoter. In some embodiments, the promoter is a constitutive promoter optionally selected from the group of the CaMV35S promoter, a derivative of the CaMV35S promoter, the maize ubiquitin promoter, the trefoil promoter, a vein mosaic cassava virus promoter, or the Arabidopsis UBQ10 promoter. In some embodiments, the plant is selected from the group of corn, rice, barley, wheat, Trema spp., apple, pear, plum, apricot, peach, almond, walnut, strawberry, raspberry, blackberry, red currant, black currant, melon, cucumber, pumpkin, squash, grape, bean, soybean, pea, chickpea, cowpea, pigeon pea, lentil, Bambara groundnut, lupin, pulses, Medicago spp., Lotus spp., forage legumes, indigo, legume trees, or hemp. In some embodiments, the plant part is a leaf, a stem, a root, a root primordia, a flower, a seed, a fruit, a kernel, a grain, a cell, or a portion thereof.

In some aspects, the present disclosure relates to a genetically altered plant or part thereof, including a first nucleic acid sequence encoding a modified plant LysM receptor where the LysM1 domain has been modified as in any of the preceding embodiments relating to modification to the LysM1 domain and a second nucleic acid sequence encoding a modified plant LysM receptor where the LysM2 domain has been modified to include a hydrophobic patch as in any of the preceding embodiments relating to modifications to the LysM2 domain. In some embodiments, the modified plant LysM receptor has higher affinity, higher selectivity, and/or altered specificity for LCOs than the unmodified plant LysM receptor and the expression of the modified plant LysM receptor allows the plant or part thereof to recognize LCOs with high affinity, high selectivity, and/or altered specificity. In some embodiments, the LCOs are produced by nitrogen-fixing bacteria or by mycorrhizal fungi. In some embodiments, the LCOs are produced by nitrogen-fixing bacteria selected from the group of Mesorhizobium loti, Mesorhizobium huakuii, Mesorhizobium mediterraneum, Mesorhizobium ciceri, Mesorhizobium spp., Rhizobium mongolense, Rhizobium tropici, Rhizobium etli phaseoli, Rhizobium giardinii, Rhizobium leguminosarum optionally R. leguminosarum trifolii, R. leguminosarum viciae, and R. leguminosarum phaseoli, Burkholderiales optionally symbionts of Mimosa, Sinorhizobium meliloti, Sinorhizobium medicae, Sinorhizobium fredii, Sinorhizobium NGR234, Azorhizobium caulinodans, Bradyrhizobium japonicum, Bradyrhizobium elkanii, Bradyrhizobium liaonginense, Frankia spp., or any combination thereof, or by or by mycorrhizal fungi selected from the group of Acaulosporaceae spp., Diversisporaceae spp., Gigasporaceae spp., Pacisporaceae spp., Funneliformis spp., Glomus spp., Rhizophagus spp., Sclerocystis spp., Septoglomus spp., Claroideoglomus spp., Ambispora spp., Archaeospora spp., Geosiphon pyriformis, Paraglomus spp., other species in the division Glomeromycota, or any combination thereof. In some embodiments, the modified polypeptide is localized to a plant cell plasma membrane. In some embodiments, the plant cell is a root cell. In some embodiments, the root cell is a root epidermal cell. In some embodiments, the modified polypeptide is expressed in a developing plant root system. In some embodiments, the first nucleic acid or second nucleic acid sequence is operably linked to a promoter. In some embodiments, the promoter is a root specific promoter. In some embodiments, the promoter is selected from the group of a NFR1/LYK3/CERK6 or NFR5/NFP promoter, the Lotus NFR5 promoter (SEQ ID NO: 24) and the Lotus NFR1 promoters (SEQ ID NO: 25) the maize allothioneine promoter, the chitinase promoter, the maize ZRP2 promoter, the tomato LeExtl promoter, the glutamine synthetase soybean root promoter, the RCC3 promoter, the rice antiquitine promoter, the LRR receptor kinase promoter, or the Arabidopsis pCO2 promoter. In some embodiments, the promoter is a constitutive promoter optionally selected from the group of the CaMV35S promoter, a derivative of the CaMV35S promoter, the maize ubiquitin promoter, the trefoil promoter, a vein mosaic cassava virus promoter, or the Arabidopsis UBQ10 promoter. In some embodiments, the plant is selected from the group of corn, rice, barley, wheat, Trema spp., apple, pear, plum, apricot, peach, almond, walnut, strawberry, raspberry, blackberry, red currant, black currant, melon, cucumber, pumpkin, squash, grape, bean, soybean, pea, chickpea, cowpea, pigeon pea, lentil, Bambara groundnut, lupin, pulses, Medicago spp., Lotus spp., forage legumes, indigo, legume trees, or hemp. In some embodiments, the plant part is a leaf, a stem, a root, a root primordia, a flower, a seed, a fruit, a kernel, a grain, a cell, or a portion thereof. In some embodiments, the plant part is a fruit, a kernel, or a grain.

In some aspects, the present disclosure relates to a pollen grain or an ovule of a genetically altered plant of any of the above embodiments relating to plants.

In some aspects, the present disclosure relates to a protoplast from a genetically altered plant of any of the above embodiments relating to plants.

In some aspects, the present disclosure relates to a tissue culture produced from protoplasts or cells from a genetically altered plant of any of the above embodiments relating to plants, wherein the cells or protoplasts are produced from a plant part selected from the group of leaf, anther, pistil, stem, petiole, root, root primordia, root tip, fruit, seed, flower, cotyledon, hypocotyl, embryo, or meristematic cell.

In some aspects, the present disclosure relates to a method of producing the genetically altered plant of any one of the above embodiments relating to plants, including introducing a genetic alteration to the plant having the nucleic acid sequence. In some embodiments, the nucleic acid sequence, the first nucleic acid sequence, and/or the second nucleic acid sequence is operably linked to a promoter. In some embodiments, the promoter is a root specific promoter. In some embodiments, the promoter is selected from the group of a NFR1/LYK3/CERK6 or NFR5/NFP promoter, the Lotus NFR5 promoter (SEQ ID NO: 24) and the Lotus NFR 1 promoters (SEQ ID NO:25) the maize allothioneine promoter, the chitinase promoter, the maize ZRP2 promoter, the tomato LeExtl promoter, the glutamine synthetase soybean root promoter, the RCC3 promoter, the rice antiquitine promoter, the LRR receptor kinase promoter, or the Arabidopsis pCO2 promoter. In some embodiments, the promoter is a constitutive promoter optionally selected from the group of the CaMV35S promoter (KAY et al. Science, 236, 4805, 1987), a derivative of the CaMV35S promoter, the maize ubiquitin promoter, the trefoil promoter, a vein mosaic cassava virus promoter, or the Arabidopsis UBQ10 promoter. In some embodiments, the nucleic acid sequence, the first nucleic acid sequence, and/or the second nucleic acid sequence is inserted into the genome of the plant so that the nucleic acid sequence, the first nucleic acid sequence, and/or the second nucleic acid sequence is operably linked to an endogenous promoter. In some embodiments, the endogenous promoter is a root specific promoter.

In further aspects, the present disclosure relates to methods for selection of a target plant LysM receptor for modifying the target plant LysM receptor to have a desired receptor characteristic, wherein the method includes the steps of: a) providing a structural model, a molecular model, a surface characteristics model, and/or an electrostatic potential model of a donor plant LysM receptor having the desired receptor characteristic and two or more potential target plant LysM receptors; b) comparing each of the two or more potential target plant LysM receptors with the structural model, the molecular model, the surface characteristics model, and/or the electrostatic potential model of the donor plant LysM receptor, and/or comparing each of the two or more potential target plant LysM receptors with the donor plant LysM receptor using structural overlay; and c) selecting the potential target plant LysM receptor with a suitable match for the donor plant LysM receptor to be the target plant LysM receptor. In some embodiments, the criteria for determining that the potential target plant LysM receptor is a suitable match for the donor plant LysM receptor in step (c) are selected from the group of goodness of fit to template structure; similarity; phylogenetic relation; surface potential; coverage to template structure; GMQE, QMEAN, and Local Quality estimates from SWISS-Model; or any combination thereof. In some embodiments, the structural model of a donor plant LysM receptor is a protein crystal structure, a molecular model, a cryo-EM structure, and a NMR structure. In some embodiments, the donor plant LysM receptor model is of an entire ectodomain and the two or more potential target plant LysM receptor models are of entire ectodomains. In some embodiments, the donor plant LysM receptor model is of a LysM1 domain, a LysM2 domain, a LysM3 domain, or any combination thereof, and the two or more potential target plant LysM receptor models are of LysM1 domains, LysM2 domains, LysM3 domains, or any combination thereof.

In some embodiments, the donor plant LysM receptor is Medicago NFP, Medicago LYK3, Lotus NFR1, Lotus NFR5, Lotus LYS11, or Arabidopsis CERK1. In some embodiments, the two or more target plant LysM receptors are additionally compared to Lotus CERK6. In some embodiments, the two or more potential target plant LysM receptor polypeptides are all from the same plant species or plant variety. In some embodiments, the desired receptor characteristic is affinity, selectivity, and/or specificity for an oligosaccharide or class of oligosaccharides. In some embodiments, the desired receptor characteristic is binding kinetics for an oligosaccharide or class of oligosaccharides, wherein the binding kinetics include off-rate and on-rate. In some embodiments, the class of oligosaccharides is selected from the group of LCOs, COs, beta-glucans, cyclic-beta-glucans, exopolysaccharides, or optionally LPS. In some embodiments, the class of oligosaccharides is LCOs or COs. In some embodiments, the class of oligosaccharides is LCOs, optionally produced by a produced by a nitrogen-fixing bacteria optionally selected from the group of Mesorhizobium loti, Mesorhizobium huakuii, Mesorhizobium mediterraneum, Mesorhizobium ciceri, Mesorhizobium spp., Rhizobium mongolense, Rhizobium tropici, Rhizobium etli phaseoli, Rhizobium giardinii, Rhizobium leguminosarum optionally R. leguminosarum trifolii, R. leguminosarum viciae, and R. leguminosarum phaseoli, Burkholderiales optionally symbionts of Mimosa, Sinorhizobium meliloti, Sinorhizobium medicae, Sinorhizobium fredii, Sinorhizobium NGR234, Azorhizobium caulinodans, Bradyrhizobium japonicum, Bradyrhizobium elkanii, Bradyrhizobium liaonginense, Frankia spp., or any combination thereof; or optionally produced by a mycorrhizal fungi optionally selected from the group of Acaulosporaceae spp., Diversisporaceae spp., Gigasporaceae spp., Pacisporaceae spp., Funneliformis spp., Glomus spp., Rhizophagus spp., Sclerocystis spp., Septoglomus spp., Claroideoglomus spp., Ambispora spp., Archaeospora spp., Geosiphon pyriformis, Paraglomus spp., other species in the division Glomeromycota, pr any combination thereof. In some embodiments, the LCOs are M. loti LCO, S. meliloti LCO-IV, or S. meliloti LCO-V.

In some embodiments, the method further includes step d) identifying one or more amino acid residues for modification in the target LysM receptor by comparing amino acid residues of a first oligosaccharide binding feature in the donor plant LysM receptor with the corresponding amino acid residues in the target plant LysM receptor, and optionally identifying one or more amino acid residues for modification in the target LysM receptor by comparing amino acid residues of a second oligosaccharide binding feature in the donor plant LysM receptor with the corresponding amino acid residues in the target plant LysM receptor. In some embodiments, the method further includes step e) generating a modified plant LysM receptor wherein the one or more amino acid residues in the first oligosaccharide binding feature of the target plant LysM receptor have been substituted with corresponding amino acid residues from the donor LysM; generating a modified plant LysM receptor wherein the one or more amino acid residues in the second oligosaccharide binding feature of the target plant LysM receptor have been substituted with corresponding amino acid residues from the donor LysM; or generating a modified plant LysM receptor wherein the one or more amino acid residues in the first oligosaccharide binding feature of the target plant LysM receptor have been substituted with corresponding amino acid residues from the donor LysM and the one or more amino acid residues in the second oligosaccharide binding feature of the target plant LysM receptor have been substituted with corresponding amino acid residues from the donor LysM. In some embodiments, the first oligosaccharide binding feature is a hydrophobic patch on the surface of the LysM2 domain. In some embodiments, the second oligosaccharide binding feature is a part of the LysM1 domain of the donor plant LysM receptor.

In additional aspects, the present disclosure relates to a a modified plant LysM receptor produced using any one of the preceding methods, wherein the modified plant LysM receptor includes a LysM2 domain modified to include a hydrophobic patch on the surface of the LysM2 domain. In some embodiments, the modified LysM2 domain binds a lipo-chitooligosaccharide (LCO). In some embodiments, the modified LysM2 domain binds the LCO with higher affinity than the unmodified LysM2 domain. In some embodiments, the modified LysM2 domain binds the LCO with higher selectivity for the LCO than the unmodified LysM2 domain. In some embodiments, the higher affinity or higher selectivity is due to the hydrophobic patch interacting with the LCO. In some embodiments, the higher affinity or higher selectivity is due to the hydrophobic patch interacting with the lipid of the LCO. In some embodiments, the LCO is produced by nitrogen-fixing bacteria or by mycorrhizal fungi. In some embodiments, the LCO is produced by nitrogen-fixing bacteria selected from the group of Mesorhizobium loti, Mesorhizobium huakuii, Mesorhizobium mediterraneum, Mesorhizobium ciceri, Mesorhizobium spp., Rhizobium mongolense, Rhizobium tropici, Rhizobium etli phaseoli, Rhizobium giardinii, Rhizobium leguminosarum optionally R. leguminosarum trifolii, R. leguminosarum viciae, and R. leguminosarum phaseoli, Burkholderiales optionally symbionts of Mimosa, Sinorhizobium meliloti, Sinorhizobium medicae, Sinorhizobium fredii, Sinorhizobium NGR234, Azorhizobium caulinodans, Bradyrhizobium japonicum, Bradyrhizobium elkanii, Bradyrhizobium liaonginense, Frankia spp., and any combination thereof, or by mycorrhizal fungi selected from the group of Acaulosporaceae spp., Diversisporaceae spp., Gigasporaceae spp., Pacisporaceae spp., Funneliformis spp., Glomus spp., Rhizophagus spp., Sclerocystis spp., Septoglomus spp., Claroideoglomus spp., Ambispora spp., Archaeospora spp., Geosiphon pyriformis, Paraglomus spp., other species in the division Glomeromycota, or any combination thereof.

In some embodiments of any of the above embodiments, the LysM receptor is selected from the group of a LysM chitooligosaccharide (CO) receptor, a LysM LCO receptor, or a LysM peptidoglycan (PGN) receptor. In some embodiments, the hydrophobic patch is adjacent to a chitin binding motif if the LysM receptor is the LysM CO receptor or the LysM LCO receptor. In some embodiments, the hydrophobic patch is within 30 Å, 20 Å, 10 Å, 7.5 Å, 5 Å, 4 Å, 3 Å, 2 Å, 1.5 Å, or 1 Å of a chitin binding motif if the LysM receptor is the LysM CO receptor or the LysM LCO receptor. In some embodiments, the hydrophobic patch is adjacent to a glycan binding motif if the LysM receptor is the LysM PGN receptor. In some embodiments, the hydrophobic patch is within 30 Å, 20 Å, 10 Å, 7.5 Å, 5 Å, 4 Å, 3 Å, 2 Å, 1.5 Å, or 1 Å of a glycan binding motif if the LysM receptor is the LysM PGN receptor. In some embodiments, the LysM receptor is not an exopolysaccharide (EPS) receptor.

In some embodiments of any of the above embodiments, the LysM receptor is a polypeptide having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO:34 (i.e., Lotus CERK6; BAI79273.1_LjCERK6). In some embodiments of any of the above embodiments, the LysM receptor is a polypeptide having amino acid sequence SEQ ID NO:34 (i.e., Lotus CERK6; BAI79273.1_LjCERK6).

In some embodiments of any of the above embodiments, the hydrophobic patch was generated by deleting at least one non-hydrophobic amino acid residue, substituting at least one amino acid residue with a more hydrophobic amino acid, or combinations thereof. In some embodiments of any of the above embodiments, the hydrophobic patch was generated by modifying an existing hydrophobic patch in the unmodified LysM receptor. In some embodiments, the unmodified LysM receptor was modified by deleting at least one non-hydrophobic amino acid residue, substituting at least one amino acid residue with a more hydrophobic amino acid, substituting at least one hydrophobic amino acid residue with another hydrophobic amino acid residue, or combinations thereof. In some embodiments, the at least one amino acid was identified by an amino acid sequence alignment with a LysM2 domain from a LysM high affinity LCO receptor that naturally has a hydrophobic patch that interacts with LCO. In some embodiments, the at least one amino acid corresponds to an amino acid that is in bold underline in FIGS. 12A-12E and FIG. 13C. In some embodiments, the at least one amino acid corresponds to an amino acid that is in bold underline in FIGS. 12A-12E and FIG. 13C or corresponds to an amino acid that is immediately N-terminal or C-terminal to an amino acid that is in bold underline in FIGS. 12A-12E and FIG. 13C. In some embodiments, the at least one amino acid corresponds to an amino acid that is in bold underline in FIGS. 12A-12E in a known LCO receptor. In some embodiments, the at least one amino acid corresponds to an amino acid that is in bold underline in FIGS. 12A-12E in a known LCO receptor or corresponds to an amino acid that is immediately N-terminal or C-terminal to an amino acid that is in bold underline in FIGS. 12A-12E in a known LCO receptor. In some embodiments, the at least one amino acid was identified by structural modelling to identify a region in LysM2 where the hydrophobic patch can be engineered. In some embodiments, the structural modeling used the unmodified plant LysM amino acid sequence and a LysM domain three dimensional structure that has a known hydrophobic patch. In some embodiments, the LysM domain three dimensional structure is a Medicago NFP ectodomain. In some embodiments, the known hydrophobic patch amino acid residues of the LysM domain three dimensional structure are or correspond to L147, L151, L152, L154, T156, K157 and V158 of the Medicago NFP ectodomain. In some embodiments, the LysM domain three dimensional structure is a Lotus LYS11 ectodomain. In some embodiments, the unmodified LysM receptor is the Lotus LYS11 receptor and the existing hydrophobic patch amino acid residues of the LysM domain that are modified are or correspond to K100, E101, G102, E103, S104, Y105, Y106, N128, and Y129 of the Lotus LYS11 ectodomain. In some embodiments, the alpha carbon of at least one amino acid was within 3 Å of an alpha carbon of a known hydrophobic patch amino acid residue in the structural alignment. In some embodiments, the structural modeling was performed using SWISS-MODEL, PDB2PQR, APBS, PyMol, and APBS tools 2.1. In some embodiments of any of the above embodiments, cither or both (i) 80% or fewer, 70% or fewer, 60% or fewer, 50% or fewer, 40% or fewer, 30% or fewer, or 20% or fewer of amino acid residues in the LysM2 domain of the unmodified LysM receptor were substituted or deleted to generate the modified plant LysM receptor, and (ii) the entire LysM2 domain in the unmodified plant LysM receptor was not substituted with another entire LysM2 domain to generate the modified plant LysM receptor. In some embodiments that may be combined with any of the preceding embodiments, the present disclosure related to a genetically altered plant or part thereof including the modified plant LysM receptor of any of the above embodiments.

In further aspects, the present disclosure relates to a a modified plant LysM receptor produced using any one of the preceding methods, wherein the modified plant LysM receptor includes a first LysM1 domain modified to replace at least part of the first LysM1 domain with at least part of a second LysM1 domain. In some embodiments, the first LysM1 domain is modified by substituting a first part of the first LysM1 domain with a third part of a second LysM1 domain and/or by substituting a second part of the first LysM1 domain with a fourth part of the second LysM1 domain. In some embodiments, the first LysM1 domain and the second LysM1 domain have different affinities, selectivities, and/or specificities for oligosaccharides and the modification of the first LysM1 domain alters the affinity, selectivity, and/or specificity to be more like the second LysM1 domain. In some embodiments, the first part and the third part correspond to SEQ ID NO:30 [Lotus CERK6 region II 43-53] or NGSNLTYISEI, SEQ ID NO: 28 [Lotus NFR1 region II 41-52] or PGVFILQNITTF; and wherein the second part and the fourth part correspond to SEQ ID NO:31 [Lotus CERK6 region IV 74-82] or ASKDSVQAG; SEQ ID NO:29 [Lotus NFR1 region IV 73-81], or LNDINIQSF. In some embodiments, the first LysM1 domain is selected from the group of SEQ ID NO:32 [LysM1 domain Lotus NFR1; LjNFR1/26-95], SEQ ID NO:33 [LysM1 domain Medicago LYK3; MtLYK3/25-95], or NFR1 DLALASYYILPGVFILQNITTFMQSEIVSSNDAITSYNKDKILNDINIQSFQRLNIPFP (SEQ ID NO: 55); and the second LysM1 domain is CERK6: ALAQASYYLLNGSNLTYISEIMQSSLLTKPEDIVSYNQDTIASKDSVQAGQRINVPFP (SEQ ID NO:107). In some embodiments, the first part is selected from SEQ ID NO:30 [Lotus CERK6 region II 43-53] or NGSNLTYISEI; the second part is selected from SEQ ID NO: 31 [Lotus CERK6 region IV 74-82] or ASKDSVQAG; the third part is selected from SEQ ID NO: 28 [Lotus NFR1 region II 41-52] or PGVFILQNITTF; and the fourth part is selected from SEQ ID NO: 29 [Lotus NFR 1 region IV 73-81] or LNDINIQSF. In some embodiments of any of the above embodiments including the first LysM1 domain being modified to replace at least part of the first LysM1 domain with at least part of a second LysM1 domain, the first LysM1 domain is further modified by substituting a fifth part of the first LysM1 domain with a sixth part of a second LysM1 domain. In some embodiments, the first LysM1 domain is SEQ ID NO:115 [LysM1 domain Lotus NFR1; LjNFR1/32-89] or SEQ ID NO: 106 [LysM1 domain Lotus NFR1; LjNFR1/31-89] and the second LysM1 domain is SEQ ID NO:114 [LysM1 domain Medicago LYK3; MtLYK3/31-89] or SEQ ID NO: 105 [LysM1 domain Medicago LYK3; MtLYK3/30-89]. In some embodiments, wherein the fifth part is SEQ ID NO:53 [Lotus NFR1 region III 59-62; LjNFR1/59-62], and wherein the sixth part is SEQ ID NO:46 [Medicago LYK3 region III 57-62; MtLYK3/57-62]. In some embodiments, the first LysM1 domain is modified by substituting a seventh part of the first LysM1 domain, wherein the seventh part spans the first part of the first LysM1 domain, the second part of the first LysM1 domain, and the fifth part of the first LysM1 domain, with an eighth part of the second LysM1 domain, wherein the eighth part spans the third part of the second LysM1 domain, the fourth part of the second LysM1 domain, and the sixth part of the second LysM1 domain. In some embodiments, the seventh part of the first LysM1 domain is SEQ ID NO:51 [Lotus NFR1 regions II-IV 41-82; LjNFR1/41-82], and the cighth part of the second LysM1 domain is SEQ ID NO:113 [Medicago LYK3 regions II-IV 40-82; MtLYK3/40-82] or SEQ ID NO: 104 [Medicago LYK3 regions II-IV 41-82; MtLYK3/41-82]. In some embodiments, the first LysM1 domain is SEQ ID NO:33 [LysM1 domain Medicago LYK3; MtLYK3/31-89] and the second LysM1 domain is SEQ ID NO:32 [LysM1 domain Lotus NFR1; LjNFR1/32-89]. In some embodiments, the fifth part is SEQ ID NO:46 [Medicago LYK3 region III 57-62; MtLYK3/57-62], and the sixth part is SEQ ID NO:53 [Lotus NFR1 region III 59-62; LjNFR1/59-62]. In some embodiments of any of the above embodiments including the first LysM1 domain being SEQ ID NO:33 and the second LysM1 domain being SEQ ID NO:32, the first LysM1 domain is modified by substituting a seventh part of the first LysM1 domain, wherein the seventh part spans the first part of the first LysM1 domain, the second part of the first LysM1 domain, and the fifth part of the first LysM1 domain, with an eighth part of the second LysM1 domain, wherein the eighth part spans the third part of the second LysM1 domain, the fourth part of the second LysM1 domain, and the sixth part of the second LysM1 domain. In some embodiments, the seventh part of the first LysM1 domain is SEQ ID NO:51 [Lotus NFR1 regions II-IV 41-82; LjNFR1/41-82], and the eighth part of the second LysM1 domain is SEQ ID NO:113 [Medicago LYK3 regions II-IV 40-82; MtLYK3/40-82] or SEQ ID NO: 104 [Medicago LYK3 regions II-IV 41-82; MtLYK3/41-82].

In some embodiments of any of the above embodiments including the first LysM1 domain being modified to replace at least part of the first LysM1 domain with at least part of a second LysM1 domain, the entire first LysM1 domain was replaced with the entire second LysM1 domain. In some embodiments of any of the above embodiments including the first LysM1 domain being modified to replace at least part of the first LysM1 domain with at least part of a second LysM1 domain, either or both (i) 80% or fewer, 70% or fewer, 60% or fewer, 50% or fewer, 40% or fewer, 30% or fewer, or 20% or fewer of amino acid residues in the first LysM1 domain were substituted or deleted with the corresponding amino acid residues of the second LysM1 domain, and (ii) the entire LysM1 domain in the unmodified plant LysM receptor was not substituted with another entire LysM2 domain to generate the modified plant LysM receptor. In some embodiments, the modified LysM1 domain binds a lipo-chitooligosaccharide (LCO) produced by nitrogen-fixing bacteria or by mycorrhizal fungi. In some embodiments, the LCO is produced by nitrogen-fixing bacteria selected from the group of Mesorhizobium loti, Mesorhizobium huakuii, Mesorhizobium mediterraneum, Mesorhizobium ciceri, Mesorhizobium spp., Rhizobium mongolense, Rhizobium tropici, Rhizobium etli phaseoli, Rhizobium giardinii, Rhizobium leguminosarum optionally R. leguminosarum trifolii, R. leguminosarum viciae, and R. leguminosarum phaseoli, Burkholderiales optionally symbionts of Mimosa, Sinorhizobium caulinodans, Bradyrhizobium japonicum, Bradyrhizobium elkanii, Bradyrhizobium liaonginense, Frankia spp., or any combination thereof, or by mycorrhizal fungi selected from the group of Acaulosporaceae spp., Diversisporaceae spp., Gigasporaceae spp., Pacisporaceae spp., Funneliformis spp., Glomus spp., Rhizophagus spp., Sclerocystis spp., Septoglomus spp., Claroideoglomus spp., Ambispora spp., Archaeospora spp., Geosiphon pyriformis, Paraglomus spp., other species in the division Glomeromycota, or any combination thereof. In some embodiments, the modified LysM1 domain binds an LCO with higher affinity than an unmodified LysM1 domain. In some embodiments, the modified LysM1 domain binds LCOs with higher selectivity than an unmodified LysM1 domain. In some embodiments, the modified LysM1 domain binds LCOs with altered specificity as compared to an unmodified LysM1 domain. In some embodiments, structural modelling was used to define the LysM1 domain and was used to identify the first part, the second part, the third part, and/or the fourth part for substitution. In some embodiments, the receptor of the above embodiments further contains a LysM2 domain modified to contain a hydrophobic patch as in any one of the previous embodiments relating to modifying the LysM2 domain. In some embodiments that may be combined with any of the preceding embodiments, the present disclosure related to a genetically altered plant or part thereof including the modified plant LysM receptor of any of the above embodiments.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

FIG. 1 shows the structure of the NFP receptor ectodomain (NFP-ECD) with the three LysM domains labeled and colored in blue (LysM1), green (LysM2), and red (LysM3). Motifs within the LysM domains are also labeled: LysM1 motifs=α1, α2, β1, and β2; LysM2 motifs=α3, α4, 3, and β4; and LysM3 motifs=α5, α6, β5, and β6. Glycosylations (di-GlcNAc cores are shown (projecting from α1 at upper; additional cores visible at center adjacent to β2 and β1 as well as at bottom left behind α4), and disulfide bridges are indicated with arrows and labeled with the residue numbers (C47-C166; C39-C104; and C102-C164).

FIGS. 2A-2B show the hydrophobic patch in the Medicago NFP LysM2 domain, and binding assay measurements using mutants of important residues within the hydrophobic patch. FIG. 2A shows molecular docking of CO4 (designated as “Ligand”) onto Medicago NFP colored with electrostatic surface potential (negative=red; hydrophobic=white; positive=blue). The hydrophobic patch is circled by a dashed black line, and the locations of important residues L147 and L154 are shown using arrows. The position of the LCO fatty-acid is depicted with a dashed orang line. FIG. 2B shows binding assay measurements comparing a wild type (WT) NFP (“NFP WT”; in green) with an NFP mutated at residues 147 and 154 (“NFP L147D L154D”; in black). The results shown for NFP WT are from seven replicates and the results shown for NFP L147D L154D are from four replicates.

FIG. 3 shows the general schematic of the construct used for mutant complementation experiments. Designations are as follows: T-DNA left border=LB, T-DNA right border=RB, nuclear localized triple yellow fluorescent protein=tYFPnls, buffer sequence=buffer, constitutive ubiquitin promoter=pUbi, Nfr1 promoter=pNfr1, Cerk6 promoter=pCerk6. The arrows indicate the directions of gene transcription.

FIGS. 4A-4B show complementation assays of Medicago nfp mutants. FIG. 4A shows complementation tested by inoculation with S. meliloti strain 2011. FIG. 4B shows complementation tested by inoculation with S. medicae. Columns represent the mean nodule numbers, while circles represent the individual counts. Empty circles=Medicago A17 wild type; filled circles=Medicago nfp mutant; EVC=empty vector control; and WT=wild type. Error bars show the SEM. Different letters indicate significant differences between the samples (ANOVA, Tukey, P<0.05).

FIGS. 5A-5B show results of functional studies measuring nodulation and defense using domain swaps between the Lotus japonicus (Lj) LCO receptor NFR1 and the Lotus japonicus (Lj) CO receptor CERK6. FIG. 5A shows complementation experiments of a Lotus nfr1-1 single mutant with different domain-swapped protein constructs. Nodules were counted on hairy root transformed L. japonicus nfr1-1 mutant roots after the indicated days post inoculation (dpi) with M. loti R7A. FIG. 5B shows complementation of a Lotus cerk6 single mutant with different domain-swapped protein constructs. Ratios of CO8 and flg22 elicited ROS peak values are plotted normalized to the wild type sample (Gifu; transformed with the empty vector) set as 1. In FIG. 5A-5B, black dots represent individual transformed plants, and error bars show the SEM. Different letters indicate significant differences among the samples (ANOVA, Tukey, P<0.01). The compositions of the recombinant receptors are shown by color-coding of the respective parts, with pink indicating LjNFR1 derived sequences and green indicating LjCERK6 derived sequences. Colored bars indicate multiple amino acid long region swaps (pink bars are LjNFR1 derived sequences; green bars are LjCERK6 derived sequences), while white bars with a red border indicate single amino acid mutations introducing a bulky amino acid (Trp) to the LysM structure. LysM domains are labelled at the left of the recombinant receptors as LysM1, LysM2, and LysM3, and transmembrane and intracellular domains are labelled at the left of the recombinant receptors as TM+IC.

FIG. 6 shows a 3D structure of the Lotus CERK6 ectodomain with the three LysM domains labeled and colored in blue (LysM1), green (LysM2), and red (LysM3). Region II and region IV in LysM1 are labeled and colored in yellow.

FIG. 7 shows complementation of Lotus japonicus nfr1-1 mutants with LjNFR1/MtLYK3 chimeras depicted at the bottom of the graph. Complementation was assayed by counting nodules formed per plant, which is shown at the top of FIG. 7. Black dots represent individual plants, columns indicate the mean values, and error bars show the SEM. Different letters indicate significant difference among the samples (ANOVA, Tukey, P<0.01). The schematics of the individual chimeric receptors tested are shown at the bottom of FIG. 7, with white indicating LjNFR1 domains, grey indicating MtLYK3 domains, and black indicating LjCERK6 domains (control). LysM domains are labelled as LysM1, LysM2, and LysM3; transmembrane and juxtamembrane domains are labelled as TM and JM; and the kinase domain is labelled as K

FIGS. 8A-8C show an alignment of selected LysM receptors from Arabidopsis thaliana (At; AT3G21630_CERK1 (SEQ ID NO:75), AT1G77630_LYP3 (SEQ ID NO:80), AT2G17120_LYP1 (SEQ ID NO:82)), Zea mays (Zm; ZM9_NP_001146346.1 (SEQ ID NO: 72)), Hordeum vulgare (Hv; HvLysMRLK4_AK369594.1 (SEQ ID NO:73)), Medicago truncatula (Mt or Medtr; Mt_LYK9_XP_003601376 (SEQ ID NO:69), Mt_LYK3_XP_003616958 (SEQ ID NO:71), Mt_LYK10_XP_003613165 (SEQ ID NO:77), Medtr5g042440.1 (SEQ ID NO:79)), Oryza sativa (Os; XP_015611967_OsCERK1 (SEQ ID NO: 74), OsCeBiP (SEQ ID NO:81)) and Lotus japonicus (Lj; BAI79273.1_CERK6 (SEQ ID NO: 34), CAE02590.1_NFR1 (SEQ ID NO:70), BAI79284.1_EPR3 (SEQ ID NO:76), CAE02597.1_NFR5 (SEQ ID NO:78)). NFR1 and NFR5 are Nod factor receptors, EPR3 is an exopolysaccharide receptor, AtLYP1 and AtLYP3 are peptidoglycan receptors, AtCERK1, OsCERK1, OsCeBIP, CERK6 are chitooligosaccharide receptors. C(x)XXXC (SEQ ID NO: 108) and CxC motifs flanking the three LysM domains are shown. LysM1 (black line), LysM2 (green line) and LysM3 (brown line) are shown. FIG. 8A shows the first two portions of the alignment including all of the LysM1 domain and part of the LysM2 domain. FIG. 8B shows the third and fourth portions of the alignment including the rest of the LysM2 domain and all of the LysM3 domain. FIG. 8C shows the fifth portion of the alignment.

FIGS. 9A-9B show an alignment of selected LysM receptors from Arabidopsis thaliana (At; AT3G21630_CERK1 (SEQ ID NO:75)), Zea mays (Zm; XP_020399958_ZM1 (SEQ ID NO:20), XP_008652982.1_ZM5 (SEQ ID NO:21), AQK73561.1_ZM7 (SEQ ID NO: 84), NP_001147981.1_ZM3 (SEQ ID NO:85), NP_001147941.2_ZM6 (SEQ ID NO:86), AQK58792.1_ZM4 (SEQ ID NO:87), ZM9_NP_001146346.1 (SEQ ID NO:72)), Hordeum vulgare (Hv; HORVU4Hr1G066170_HvLysMRLK10 (SEQ ID NO:19), AK357612_HvLysMRLK2 (SEQ ID NO:17), AK370300_HvLysmRLKI (SEQ ID NO:16), AK372128_HvLysMRLK3 (SEQ ID NO:18), HvLysMRLK4_AK369594.1 (SEQ ID NO:73)), Oryza sativa (Os; XP_015611967_OsCERK1 (SEQ ID NO:74)), Medicago truncatula (Mt; XP_003613904.2_MtNFP (SEQ ID NO:83), Mt_LYK3_XP_003616958 (SEQ ID NO:71), Mt_LYK9_XP_003601376 (SEQ ID NO:69)), and Lotus japonicus (Lj; CAE02590.1_NFR 1 (SEQ ID NO:70), CAE02597.1_NFR5 (SEQ ID NO:78), BAI79273.1_CERK6 (SEQ ID NO: 34),). LjNFR1, LjNFR5, MtLYK3 and MtNFP are functional Nod factor receptors, AtCERK1, OsCERK1, LjCERK6 are functional chitin receptors. C(x)XXXC (SEQ ID NO:108) and CxC motifs flanking the three LysM domains are shown. LysM1 (black line), LysM2 (green line) and LysM3 (brown line) are shown. The number of “X” residues in the C(x)XXXC motif (SEQ ID NO: 108) located before LysM1 varies between receptors and therefore the location of LysM1 (black line) changes accordingly in the alignments in this figure and in successive figures. FIG. 9A shows the first and second portions of the alignment including all of the LysM1 domain and part of the LysM2 domain. FIG. 9B shows the third and fourth portions of the alignment including the rest of the LysM2 domain and all of the LysM3 domain.

FIGS. 10A-10B show an alignment of selected LysM receptors from Zea mays (Zm; ONM41523.1_ZM8 (SEQ ID NO:88), XP_008657477.1_ZM2 (SEQ ID NO:89), Zm00001d043516 ZM10 (SEQ ID NO:91)), Hordeum vulgare (Hv; MLOC_5489.2_HvLysM-RLK9 (SEQ ID NO:90), MLOC_18610.1_HvLysM-RLK8 (SEQ ID NO:92), MLOC_57536.1 HvLysM-RLK6 (SEQ ID NO:93)), Medicago truncatula (Mt; Mt_LYK10_XP_003613165 (SEQ ID NO:77), Mt_LYK3_XP_003616958 (SEQ ID NO:71), XP_003613904.2_MtNFP (SEQ ID NO: 83)), and Lotus japonicus (Lj; BAI79284.1_EPR3 (SEQ ID NO:76), CAE02590.1 NFR1 (SEQ ID NO:70), CAE02597.1_NFR5 (SEQ ID NO:78)). LjNFR1, LjNFR5, MtLYK3 and MtNFP are functional Nod factor receptors, LjEPR3 is functional EPS receptor. C(x)XXXC (SEQ ID NO:108) and CxC motifs flanking the three LysM domains are shown. LysM1 (black line), LysM2 (green line) and LysM3 (brown line) are shown. FIG. 10A shows the first, second, and third portions of the alignment including all of the LysM1 domain and all of the LysM2 domain. FIG. 10B shows the fourth, fifth, and sixth portions of the alignment including all of the LysM3 domain.

FIGS. 11A-11B show an alignment of selected LysM receptors from Arabidopsis thaliana (At; AT1G21880.2_LYP2 (SEQ ID NO:94), AT1G77630_LYP3 (SEQ ID NO:80), AT2G17120_LYP1 (SEQ ID NO:82)), Oryza sativa (Os; OsCeBiP (SEQ ID NO:81)), and Lotus japonicus (Lj; LjLYP1 (SEQ ID NO:95), LjLYP2 (SEQ ID NO:96), LjLYP3 (SEQ ID NO:97), CAE02590.1_NFR1 (SEQ ID NO:70), CAE02597.1_NFR5 (SEQ ID NO:78)). LjNFR1, LjNFR5, are functional Nod factor receptors, AtLYP2 and AtLYP3, are PGN receptors, OsCeBiP is a functional chitin receptor. C(x)XXXC (SEQ ID NO:108) and CxC motifs flanking the three LysM domains are shown. LysM1 (black line), LysM2 (green line) and LysM3 (brown line) are shown. FIG. 11A shows the first, second, third, and fourth portions of the alignment including all of the LysM1 domain, all of the LysM2 domain, and all of the LysM3 domain. FIG. 11B shows the fifth, sixth, and seventh portions of the alignment.

FIGS. 12A-12E show annotated amino acid sequences of previously known LCO receptors and newly identified LCO receptors. FIG. 12A shows the annotation key; the LysM1 domain is shown with a dashed underline, the LysM2 domain is shown with a solid underline, the hydrophobic patch residues are shown in bold, and the LysM3 domain is shown with residues italicized. Medicago NFP (MtNFP/1-595; SEQ ID NO:1), Lotus NFR5 (a known LCO receptor; LjNFR5/1-595; SEQ ID NO:2), Pea SYM10 (a known LCO receptor; Pea_SYM10/1-594; SEQ ID NO: 3), and Soybean NFR5α (a known LCO receptor; GmNFR5α/1-598 max; SEQ ID NO:4) are shown. FIG. 12B shows Chickpea NFR5 (a new LCO receptor; ChickpeaNFR5/1-557 (Cicer arictinum); SEQ ID NO:5), Bean NFR5 (a new LCO receptor; BeanNFR5/1-597 (Phaseolus vulgaris); SEQ ID NO:7), Peanut NFR5 (a new LCO receptor; PeanutNFR5/1-595 [Arachis hypogaca subsp. hypogaca]; SEQ ID NO:9), and Lotus LYS11 (a new LCO receptor; LjLYS11/1-591; SEQ ID NO:11). FIG. 12C shows Medicago LYRI (a new LCO receptor; MtLYR1/1-590; SEQ ID NO:12), Parasponia NFP1 (a new LCO receptor; PanNFP1/1-613; SEQ ID NO: 13), Parasponia NFP2 (a known LCO receptor; PanNFP2/1-582; SEQ ID NO:14), and Barley receptor HvLysM-RLK1 (a new LCO receptor; HvLysM-RLK1 (AK370300); SEQ ID NO: 16). FIG. 12D shows Barley receptor HvLysM-RLK2 (a new LCO receptor; HvLysM-RLK2 (AK357612); SEQ ID NO:17), Barley receptor HvLysM-RLK3 AK372128 (a new LCO receptor; HvLysM-RLK3 AK372128; SEQ ID NO:18), Barley receptor HvLysM-RLK10 (a new LCO receptor; HvLysM-RLK10 (HORVU4Hr1G066170); SEQ ID NO:19), and Maize receptor ZM1 (a new LCO receptor; ZM1 (XP_020399958); SEQ ID NO:20). FIG. 12E shows Maize receptor ZM5 (a new LCO receptor; ZM5 (XP_008652982.1); SEQ ID NO:21), Apple NFP5 (a new LCO receptor; XP_008338966.1 PREDICTED: serine/threonine receptor-like kinase NFP [Malus domestica]; SEQ ID NO:22), and Strawberry NFR5 (a new LCO receptor; XP_004300586.2 PREDICTED: protein LYK5-like [Fragaria vesca subsp. vesca]; SEQ ID NO: 23).

FIGS. 13A-13C show structural modelling of the HvLysM-RLK2/37-247 LysM1-3 domains and selection of residues for modification to introduce a hydrophobic patch. FIG. 13A shows the PyMol visualization of the LysM1-3 domains of the HvLysM-RLK2/37-247 model with the LysM1 domain labeled and in blue, the LysM2 labeled and in green, and the LysM3 labeled and in light grey. FIG. 13B shows the electrostatic surface potential of the model with chitin modeled in the binding groove. FIG. 13C has the amino acid sequence of the HvLysM-RLK2/37-247 LysM1-3 domains (SEQ ID NO: 98) with the LysM1 domain with a dashed underline, the LysM2 domain with a solid underline, and the LysM3 domain with no underline, and the residues that can be modified to create the hydrophobic patch in bold.

FIG. 14 shows an alignment of the Lotus japonicus (Lj) NFR1 and Lotus japonicus (Lj) CERK6 LysM1 domains. Viewed top-down, LjNFR1 (NFR1_Lj2g2904690; SEQ ID NO: 99) is shown in the top row, LjCERK6 (CERK6_AB503687; SEQ ID NO:100) is shown in the second row, sequence conservation is shown in the third row. Region II and region IV in the LysM1 domain are denoted by light grey arrows and labelled as “II.” or “IV.”

FIGS. 15A-15F show L. japonicus LjLYS11 ectodomain model and crystal structure, modified LjLYS11 ectodomains, and testing of modified LjLYS11 ectodomains. FIG. 15A shows a comparison of the LjLYS11 ectodomain model (LYS11-model; left) with the crystal structure of the LjLYS11 ectodomain (LYS11-crystal structure; right). FIG. 15B shows schematics of modified LjLYS11 ectodomains (LjLYS11-LjNFR5 chimeras) used for testing. The top schematic shows an ectodomain with entirely LjLYS11 domains (black), the middle schematic shows an ectodomain where the LysM2 domain from LjLYS11 was replaced with the LysM2 domain from LjNFR5 (grey), and the bottom schematic shows an ectodomain where key residues from LjLYS11 were replaced with key residues from LjNFR5 (grey) (N-terminus=N′; LysM1=M1; LysM2=M2; LysM3=M3; 6×HIS tag used for purification=6×HIS; C-terminus=C′). FIG. 15C shows the results of binding assays with the ectodomain with entirely LjLYS11 components (ectodomain schematic shown at top with LjLYS11 domains in black; results of binding assays shown at bottom). The Kd is shown in the title of each graph (CO5 (Kd=11.4 μM), M. loti LCO (Kd=38.6 μM), and S. meliloti LCO (weak binding)). FIG. 15D shows the results of binding assays with the ectodomain where LysM2 from LjLYS11 was replaced with LysM2 from LjNFR5 (ectodomain schematic shown at top with LjLYS11 domains in black and LjNFR5 domains in grey; results of binding assays shown at bottom). FIG. 15E shows the results of binding assays with the ectodomain where key residues from LjLYS11 were replaced with key residues from LjNFR5 (ectodomain schematic shown at top with LjLYS11 domains in black and LjNFR5 residues in grey; results of binding assays shown at bottom). For FIGS. 15C-15E, binding in nm is shown on the y-axes, time in seconds(s) is shown on the x-axes, and the tested molecules are shown in the titles of the graphs (CO5, M. loti LCO, and S. meliloti LCO). FIG. 15F shows complementation of L. japonicus nfr5 (Ljnfr5) mutants with LjNFR5/LjLYS11 chimeras depicted at the bottom of the graph. Complementation was assayed by counting nodules formed per plant, which is shown at the top of FIG. 15F. Black dots represent individual plants, columns indicate the mean values, and error bars show the SEM. Different letters indicate significant difference among the samples (ANOVA, Tukey, P<0.01). The schematics of the individual chimeric ectodomains tested are shown at the bottom of FIG. 15F, with light grey indicating LjNFR5 domains, grey indicating LjLYS11 domains, and empty vector denoted by a label (LysM1, LysM2 and LysM3 are shown as boxes; transmembrane domain is shown as a wavy shape; kinase domain is shown as an oval shape). Below the receptor schematics, the number of plants (Plant), the number of plants without nodules (neg), the number of plants with nodules (pos), and the frequency (freq) of plants forming nodules when transformed with the depicted vector is provided.

FIGS. 16A-16H show homology modelling of the barley RLK10 receptor (HvRLK10) ectodomain and of the barley RLK4 receptor (HvRLK4) ectodomain as well as results of binding experiments using the HvRLK10 ectodomain and the HvRLK4 ectodomain. FIG. 16A shows a schematic of the purified HvRLK10 ectodomain at the top (N-terminus=N′; LysM1=M1; LysM2=M2; LysM3=M3; 6×HIS tag used for purification=6×HIS; C-terminus=C′) and the results of binding assays of HvRLK10 ectodomain with CO5 at the bottom. FIG. 16B shows homology modelling of the Barley receptor RLK10 (HvRLK10) ectodomain with surface representation shaded according to its electrostatic potential. The hydrophobic patch is circled by a dashed black line, and the ligand is shown at the top of the hydrophobic patch. FIG. 16C shows the results of binding assays of HvRLK10 ectodomain with M. loti LCO. FIG. 16D shows the results of binding assays of HvRLK10 ectodomain with S. meliloti LCO. FIG. 16E shows a schematic of the purified HvRLK4 ectodomain at the top (N-terminus=N′; LysM1=M1; LysM2=M2; LysM3=M3; 6×HIS tag used for purification=6×HIS; C-terminus=C′) and the results of binding assays of HvRLK4 ectodomain with CO5 at the bottom. FIG. 16F shows a 3D structure of the HvRLK4 ectodomain with a ligand shown at the top. FIG. 16G shows the results of binding assays of HvRLK4 ectodomain with M. loti LCO. FIG. 16H shows the results of binding assays of HvRLK4 ectodomain with S. meliloti LCO. For FIGS. 16A, 16C-16D, 16E, and 16G-16H, binding in nm is shown on the y-axes, time in seconds(s) is shown on the x-axes, and the tested molecules are shown in the titles of the graphs (CO5, M. loti LCO, and S. meliloti LCO).

FIGS. 17A-17B show complementation of Medicago truncatula lyk3 mutants with LjNFR1/MtLYK3 chimeras as well as the MtLYK3 ectodomain structure and schematics of LjNFR1/MtLYK3 chimeras. FIG. 17A shows complementation of Medicago lyk3 mutants (Mtlyk3) with LjNFR1/MtLYK3 chimeras depicted at the bottom of the graph. The controls Medicago wild type (WT) transformed with empty vector and Mtlyk3 transformed with empty vector were included. Complementation was assayed by counting nodules formed per plant, which is shown at the top of FIG. 17A. Black dots represent individual plants, columns indicate the mean values, and error bars show the SEM. Different letters indicate significant difference among the samples (ANOVA, Tukey, P<0.01). The schematics of the individual chimeric receptors tested are shown at the bottom of FIG. 17A, with grey indicating MtLYK3 domains, black indicating LjNFR1 domains, and empty vector denoted by a label. Below the receptor schematics, the frequency (freq) of plants forming nodules when transformed with the depicted vector is provided. LysM domains are labelled as LysM1, LysM2, and LysM3; transmembrane domain is labelled as TM; and the kinase domain is labelled as kinase. FIG. 17B shows the MtLYK3 ectodomain structure, with Region II (dark grey helix), Region III (light grey helix), and Region IV (grey linker) labeled (top); and schematic representation of the engineered LjNFR1 ectodomain (black) with indicated regions from the MtLYK3 ectodomain (grey; regions from the MtLYK3 ectodomain=regions II-IV (“2 to 4”), regions II, III, and IV (“2,3,4”), and regions II and IV (“2,4”)) (bottom).

FIGS. 18A-18F show BLI binding curves for A. thaliana CERK1 (A/CERK1) binding to chitopentaose (CO5) and chitooctaose (CO8), models of CO and LCO perception, and structural alignment of the ectodomains of Medicago NFP, Arabidopsis CERK1 and Lotus CERK6. FIG. 18A shows ArCERK1 binding to chitopentaose (Chitin (CO5)). FIG. 18B shows AtCERK1 binding to chitooctaose (Chitin (CO8)). For FIGS. 18A-18B, seven 2-fold dilution series of analyte (1.56-100 M) were used for each experiment; experimental binding curves are represented in solid lines, fitting curves in dashed lines; goodness of fit is described by the global fit R2 on the mean value of each point; number of replicates performed using independent protein preparations (n) indicated; and kinetic parameters (kₒₙ and k_{off}) are shown. FIG. 18C shows a model of CO perception by CO receptors (e.g., CERK1, LYK5). FIG. 18D shows a model of LCO perception by LCO receptors (e.g., NFP, LYK). FIG. 18E shows a model of LCO perception by hydrophobic patch mutant LCO receptors (e.g., NFP, LYK). FIG. 18F shows structural alignment of the ectodomains of Medicago NFP, Arabidopsis CERK1 and Lotus CERK6. Molecular fits (RMSD values) based on structural superposition of the ectodomains are shown in Å (Angstrom). The structures (above) are shaded according to the schematic representation of the ectodomain (below). The conserved disulfide connectivity pattern between Medicago NFP, Arabidopsis CERK1 and Lotus CERK6 is highlighted.

FIGS. 19A-19D show BLI binding curves for WT NFP-ECD and hydrophobic patch mutant NFP-ECD (L147D/L154D) binding to S. meliloti LCO-IV and a schematic of the NFP receptor. FIG. 19A shows WT NFP-ECD binding to S. meliloti LCO-IV. FIG. 19B shows L147D/L154D NFP-ECD binding to S. meliloti LCO-IV. For FIGS. 19A-19B, seven 2-fold dilution series of analyte (1.56-100 μM) were used for each experiment; and experimental binding curves are represented in solid lines, fitting curves in dashed lines. FIG. 19C shows a table summarizing the kinetic parameters of FIGS. 19A-19B, with goodness of fit described by the global fit R2 on the mean value of each point, and number of replicates performed using independent protein preparations (n) indicated. FIG. 19D shows a schematic of the NFP receptor with LysM1, LysM2, LysM3, stem, and transmembrane (TM) and kinase domains labeled, and the location of the hydrophobic patch in LysM2 indicated by a grey bar. Numbers below the schematic provide the corresponding amino acid residues, and the locations of the CxC motifs flanking the LysM domains are shown.

### DETAILED DESCRIPTION

The following description sets forth exemplary methods, parameters, and the like. It should be recognized, however, that such description is not intended as a limitation on the scope of the present disclosure but is instead provided as a description of exemplary embodiments.

### Modified Plant LysM Receptors

Certain aspects of the present disclosure relate to a modified plant LysM receptor comprising a LysM2 domain modified to comprise a hydrophobic patch on the surface of the LysM2 domain. In some embodiments, the modified LysM2 domain binds a lipo-chitooligosaccharide (LCO). In some embodiments, the modified LysM2 domain binds the LCO with higher affinity than the unmodified LysM2 domain. In some embodiments, the modified LysM2 domain binds the LCO with higher selectivity for the LCO than the unmodified LysM2 domain. In some embodiments, the higher affinity or higher selectivity is due to the hydrophobic patch interacting with the LCO. In some embodiments, the higher affinity or higher selectivity is due to the hydrophobic patch interacting with the lipid of the LCO. In some embodiments, the LCO is produced by nitrogen-fixing bacteria or by mycorrhizal fungi. In some embodiments, the LCO is produced by nitrogen-fixing bacteria selected from the group of Mesorhizobium loti, Mesorhizobium huakuii, Mesorhizobium mediterraneum, Mesorhizobium ciceri, Mesorhizobium spp., Rhizobium mongolense, Rhizobium tropici, Rhizobium etli phaseoli, Rhizobium giardinii, Rhizobium leguminosarum optionally R. leguminosarum trifolii, R. leguminosarum viciae, and R. leguminosarum phaseoli, Burkholderiales optionally symbionts of Mimosa, Sinorhizobium meliloti, Sinorhizobium medicae, Sinorhizobium fredii, Sinorhizobium NGR234, Azorhizobium caulinodans, Bradyrhizobium japonicum, Bradyrhizobium elkanii, Bradyrhizobium liaonginense, Frankia spp., and any combination thereof, or by mycorrhizal fungi selected from the group consisting of Acaulosporaceae spp., Diversisporaceae spp., Gigasporaceae spp., Pacisporaceae spp., Funneliformis spp., Glomus spp., Rhizophagus spp., Sclerocystis spp., Septoglomus spp., Claroideoglomus spp., Ambispora spp., Archaeospora spp., Geosiphon pyriformis, Paraglomus spp., other species in the division Glomeromycota, or any combination thereof.

In some embodiments of any of the above embodiments, the LysM receptor is selected from the group consisting of a LysM chitooligosaccharide (CO) receptor, a LysM LCO receptor, and a LysM peptidoglycan (PGN) receptor. In some embodiments, the hydrophobic patch is adjacent to a chitin binding motif if the LysM receptor is the LysM CO receptor or the LysM LCO receptor. In some embodiments, the hydrophobic patch is within 30 Å, 29 Å, 28 Å, 27 Å, 26 Å, 25 Å, 24 Å, 23 Å, 22 Å, 21 Å, 20 Å, 19 Å, 18 Å, 17 Å, 16 Å, 15 Å, 14 Å, 13 Å, 12 Å, 11 Å, 10 Å, 9.5 Å, 9 Å, 8.5 Å, 8 Å, 7.5 Å, 7 Å, 6.5 Å, 6 Å, 5.5 Å, 5 Å, 4.5 Å, 4 Å, 3.5 Å, 3 Å, 2.5 Å, 2 Å, 1.5 Å, or 1 Å of a chitin binding motif if the LysM receptor is the LysM CO receptor or the LysM LCO receptor. In some embodiments, the hydrophobic patch is adjacent to a glycan binding motif if the LysM receptor is the LysM PGN receptor. In some embodiments, the hydrophobic patch is within 30 Å, 29 Å, 28 Å, 27 Å, 26 Å, 25 Å, 24 Å, 23 Å, 22 Å, 21 Å, 20 Å, 19 Å, 18 Å, 17 Å, 16 Å, 15 Å, 14 Å, 13 Å, 12 Å, 11 Å, 10 Å, 9.5 Å, 9 Å, 8.5 Å, 8 Å, 7.5 Å, 7 Å, 6.5 Å, 6 Å, 5.5 Å, 5 Å, 4.5 Å, 4 Å, 3.5 Å, 3 Å, 2.5 Å, 2 Å, 1.5 Å, or 1 Å of a glycan binding motif if the LysM receptor is the LysM PGN receptor. In some embodiments, the LysM receptor is not an exopolysaccharide (EPS) receptor.

In some embodiments of any of the above embodiments, the LysM receptor is a polypeptide with at least 70% sequence identity, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to SEQ ID NO:34 (i.e., Lotus CERK6; BAI79273.1_LjCERK6). In some embodiments of any of the above embodiments, the LysM receptor is a polypeptide having amino acid sequence SEQ ID NO:34 (i.e., Lotus CERK6; BAI79273.1_LJCERK6).

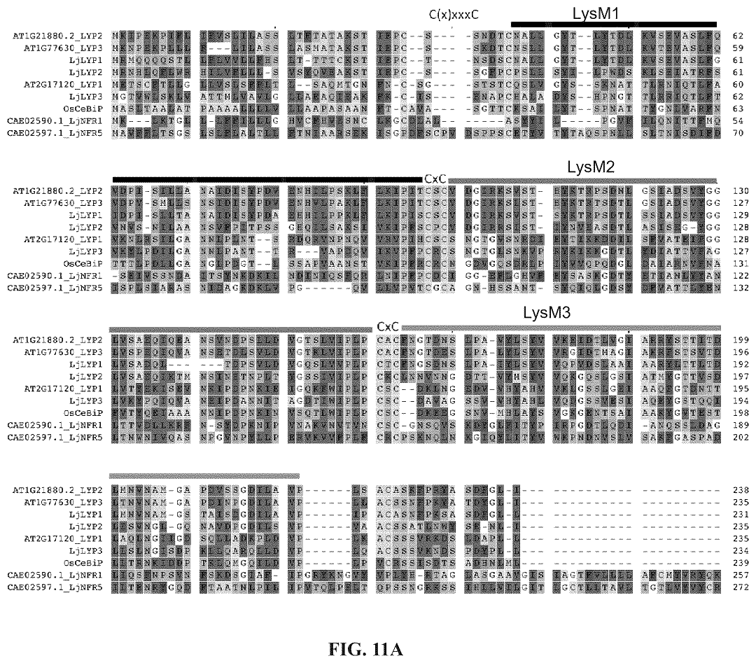

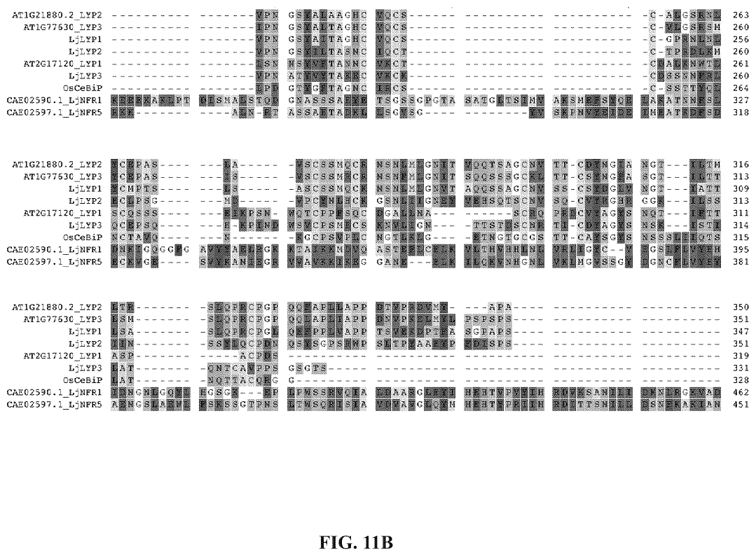

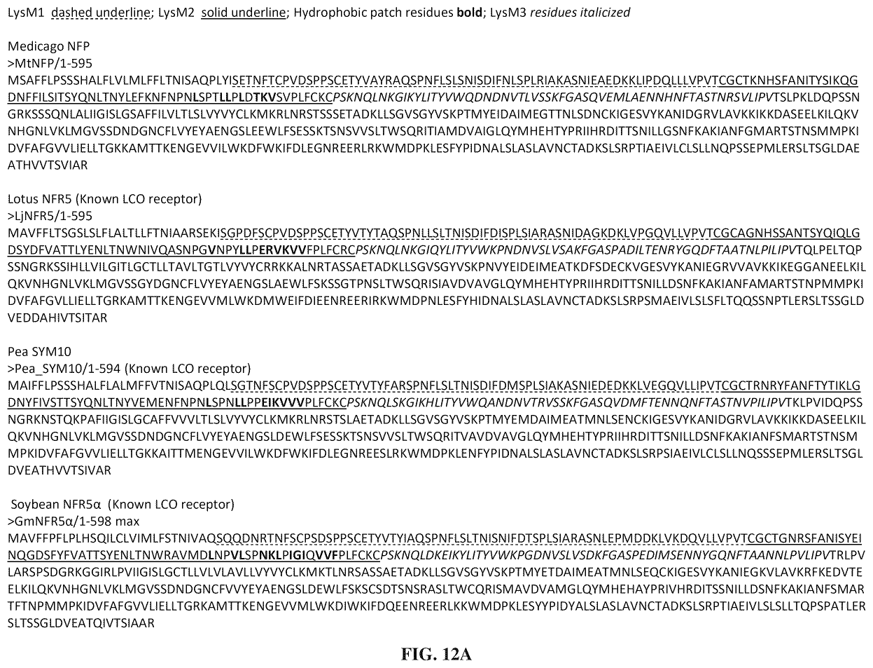

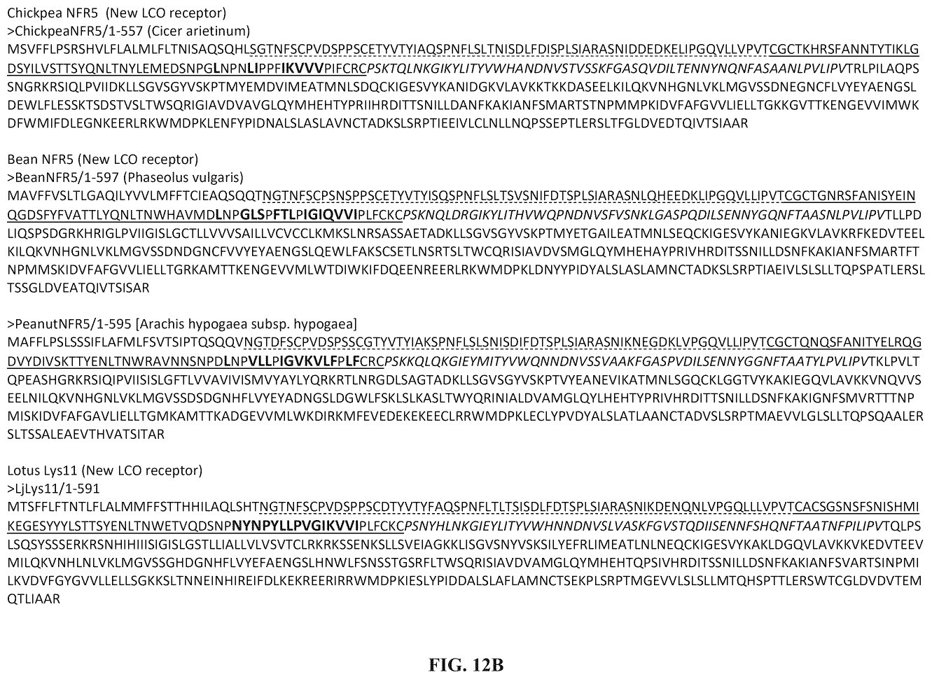

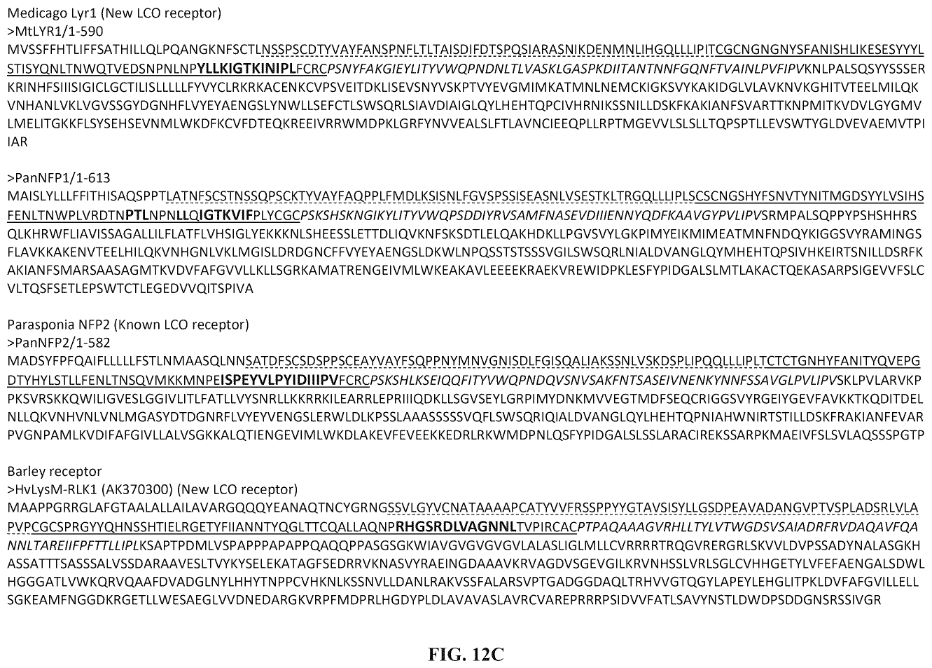

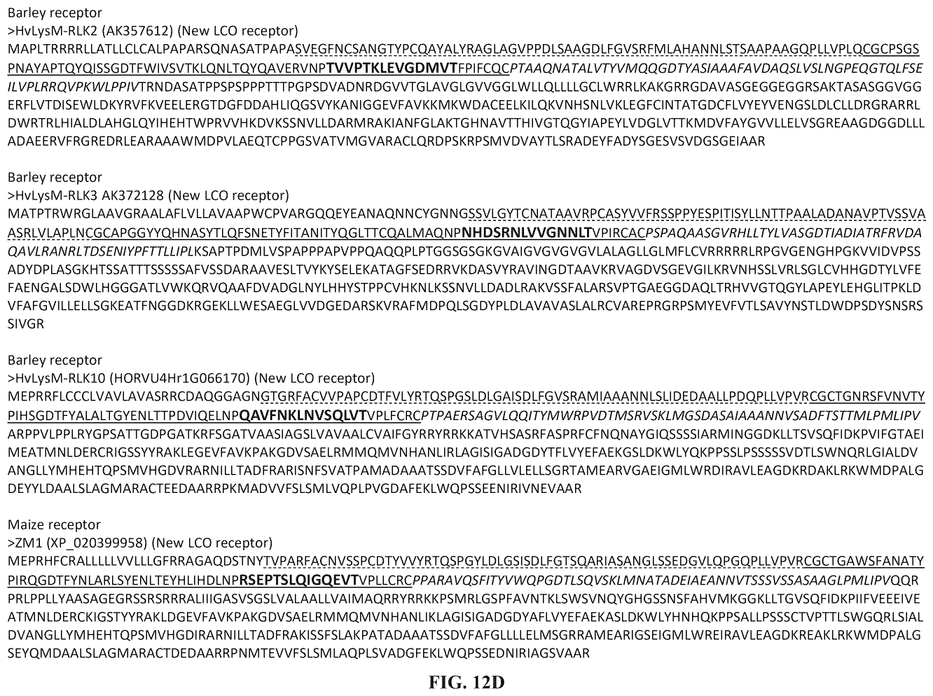

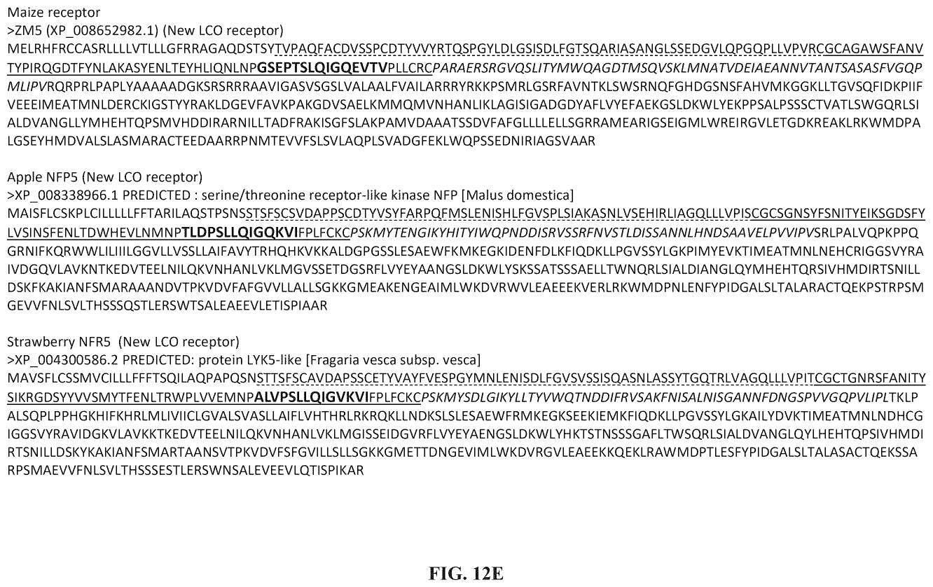

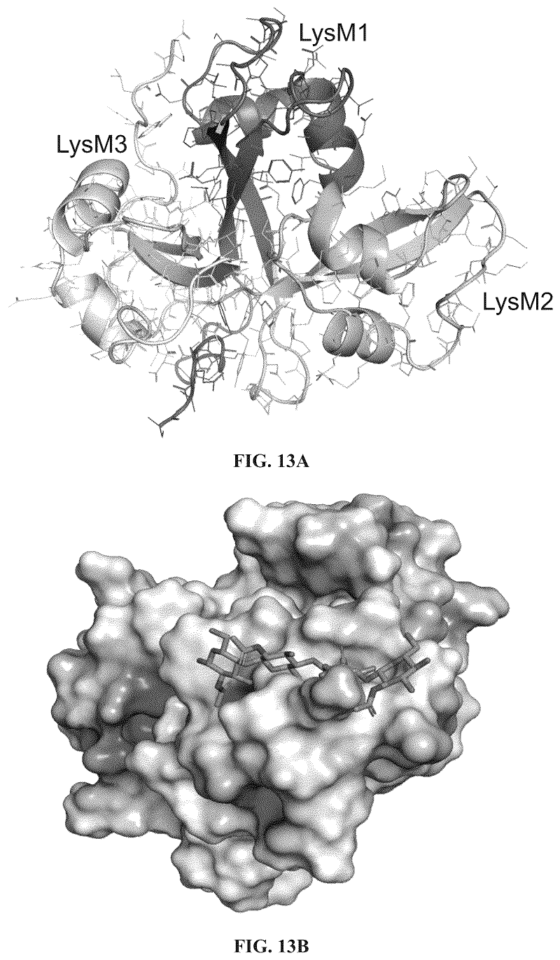

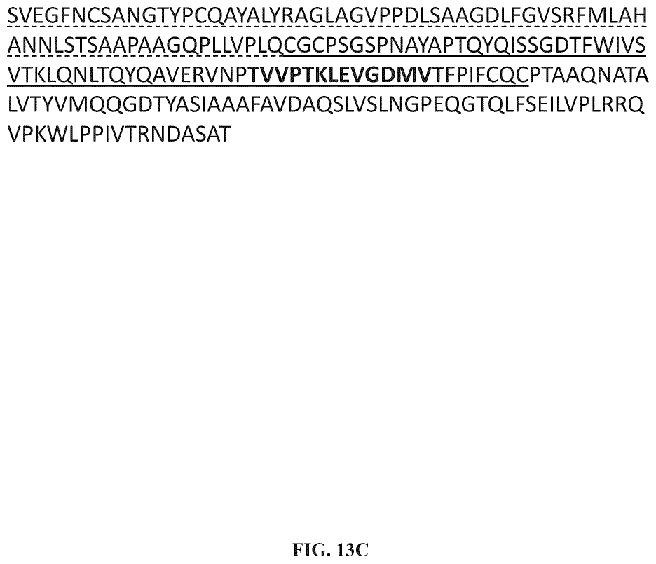

In some embodiments of any of the above embodiments, the hydrophobic patch was generated by deleting at least one non-hydrophobic amino acid residue, substituting at least one amino acid residue with a more hydrophobic amino acid, or combinations thereof. In some embodiments, the at least one amino acid was identified by an amino acid sequence alignment with a LysM2 domain from a LysM high affinity LCO receptor that naturally has a hydrophobic patch that interacts with LCO. In some embodiments, the at least one amino acid corresponds to an amino acid that is in bold in FIGS. 12A-12E and FIG. 13C. In some embodiments, the at least one amino acid corresponds to an amino acid that is in bold in FIGS. 12A-12E and FIG. 13C or corresponds to an amino acid that is immediately N-terminal or C-terminal to an amino acid that is in bold in FIGS. 12A-12E and FIG. 13C. In some embodiments, the at least one amino acid corresponds to an amino acid that is in bold in FIGS. 12A-12E in a known LCO receptor. In some embodiments, the at least one amino acid corresponds to an amino acid that is in bold in FIGS. 12A-12E in a known LCO receptor or corresponds to an amino acid that is immediately N-terminal or C-terminal to an amino acid that is in bold in FIGS. 12A-12E in a known LCO receptor. In some embodiments, the at least one amino acid was identified by structural modelling to identify a region in LysM2 where the hydrophobic patch can be engineered. In some embodiments, the structural modeling used the unmodified plant LysM amino acid sequence and a LysM domain three dimensional structure that has a known hydrophobic patch. In some embodiments, the LysM domain three dimensional structure is a Medicago NFP ectodomain. In some embodiments, the known hydrophobic patch amino acid residues of the LysM domain three dimensional structure are or correspond to L147, L151, L152, L154, T156, K157 and V158 of the Medicago NFP ectodomain. In some embodiments, the alpha carbon of at least one amino acid was within 3 Å of an alpha carbon of a known hydrophobic patch amino acid residue in the structural alignment. In some embodiments, the structural modeling was performed using SWISS-MODEL, PDB2PQR, APBS, PyMol, and APBS tools 2.1.

In some aspects, the present disclosure relates to a modified plant LysM receptor comprising a first LysM1 domain modified to replace at least part of the first LysM1 domain with at least part of a second LysM1 domain. In some embodiments, the first LysM1 domain is modified by substituting a first part of the first LysM1 domain with a third part of a second LysM1 domain and/or by substituting a second part of the first LysM1 domain with a fourth part of the second LysM1 domain. In some embodiments, the first LysM1 domain and the second LysM1 domain have different affinities and/or selectivities for oligosaccharides and the modification of the first LysM1 domain alters the affinity, selectivity, and/or specificity to be more like the second LysM1 domain. In some embodiments, the first part and the third part correspond to SEQ ID NO:30 [Lotus CERK6 region II 43-53] or NGSNLTYISEI, SEQ ID NO: 28 [Lotus NFR1 region II 41-52] or PGVFILQNITTF; and wherein the second part and the fourth part correspond to SEQ ID NO:31 [Lotus CERK6 region IV 74-82] or ASKDSVQAG; SEQ ID NO:29 [Lotus NFR1 region IV 73-81], or LNDINIQSF. In some embodiments, the first LysM1 domain is selected from the group of SEQ ID NO:32 [LysM1 domain Lotus NFR1; LjNFR1/26-95], SEQ ID NO:33 [LysM1 domain Medicago LYK3; MtLYK3/25-95], or NFR1 DLALASYYILPGVFILQNITTFMQSEIVSSNDAITSYNKD KILNDINIQSFORLNIPFP (SEQ ID NO: 55); and the second LysM1 domain is CERK6: ALAQASYYLLNGSNLTYISEIMQSSLLTKPEDIVSYNQDTIASKDSVQAGQRINVPFP (SEQ ID NO:107). In some embodiments, the first part is selected from SEQ ID NO:30 [Lotus CERK6 region II 43-53] or NGSNLTYISEI; the second part is selected from SEQ ID NO:31 [Lotus CERK6 region IV 74-82] or ASKDSVQAG; the third part is selected from SEQ ID NO: 28 [Lotus NFR1 region II 41-52] or PGVFILQNITTF; and the fourth part is selected from SEQ ID NO:29 [Lotus NFR1 region IV 73-81] or LNDINIQSF. In some embodiments, the entire first LysM1 domain was replaced with the entire second LysM1 domain. In some embodiments, the modified LysM1 domain binds a lipo-chitooligosaccharide (LCO) produced by nitrogen-fixing bacteria or by mycorrhizal fungi. In some embodiments, the LCO is produced by nitrogen-fixing bacteria selected from the group consisting of Mesorhizobium loti, Mesorhizobium huakuii, Mesorhizobium mediterraneum, Mesorhizobium ciceri, Mesorhizobium spp., Rhizobium mongolense, Rhizobium tropici, Rhizobium etli phaseoli, Rhizobium giardinii, Rhizobium leguminosarum optionally R. leguminosarum trifolii, R. leguminosarum viciae, and R. leguminosarum phaseoli, Burkholderiales optionally symbionts of Mimosa, Sinorhizobium caulinodans, Bradyrhizobium japonicum, Bradyrhizobium elkanii, Bradyrhizobium liaonginense, Frankia spp., and any combination thereof, or by mycorrhizal fungi selected from the group consisting of Acaulosporaceae spp., Diversisporaceae spp., Gigasporaceae spp., Pacisporaceae spp., Funneliformis spp., Glomus spp., Rhizophagus spp., Sclerocystis spp., Septoglomus spp., Claroideoglomus spp., Ambispora spp., Archaeospora spp., Geosiphon pyriformis, Paraglomus spp., other species in the division Glomeromycota, and any combination thereof. In some embodiments, the modified LysM1 domain binds an LCO with higher affinity than an unmodified LysM1 domain. In some embodiments, the modified LysM1 domain binds LCOs with higher selectivity than an unmodified LysM1 domain. In some embodiments, the modified LysM1 domain binds LCOs with altered specificity as compared to an unmodified LysM1 domain. In some embodiments, structural modelling was used to define the LysM1 domain and was used to identify the first part, the second part, the third part, and/or the fourth part for substitution. In some embodiments, the receptor of the above embodiments further contains a LysM2 domain modified to contain a hydrophobic patch as in any one of the previous embodiments relating to modifications to the LysM2 domain.

Additional aspects of the present disclosure relate to a modified plant LysM receptor including a LysM2 domain modified to include a hydrophobic patch on the surface of the LysM2 domain. In some embodiments, the modified LysM2 domain binds a lipo-chitooligosaccharide (LCO). In some embodiments, the modified LysM2 domain binds the LCO with higher affinity than the unmodified LysM2 domain. In some embodiments, the modified LysM2 domain binds the LCO with higher selectivity for the LCO than the unmodified LysM2 domain. In some embodiments, the higher affinity or higher selectivity is due to the hydrophobic patch interacting with the LCO. In some embodiments, the higher affinity or higher selectivity is due to the hydrophobic patch interacting with the lipid of the LCO. In some embodiments, the LCO is produced by nitrogen-fixing bacteria or by mycorrhizal fungi. In some embodiments, the LCO is produced by nitrogen-fixing bacteria selected from the group of Mesorhizobium loti, Mesorhizobium huakuii, Mesorhizobium mediterraneum, Mesorhizobium ciceri, Mesorhizobium spp., Rhizobium mongolense, Rhizobium tropici, Rhizobium etli phaseoli, Rhizobium giardinii, Rhizobium leguminosarum optionally R. leguminosarum trifolii, R. leguminosarum viciae, and R. leguminosarum phaseoli, Burkholderiales optionally symbionts of Mimosa, Sinorhizobium caulinodans, Bradyrhizobium japonicum, Bradyrhizobium elkanii, Bradyrhizobium liaonginense, Frankia spp., or any combination thereof, or by mycorrhizal fungi selected from the group of Acaulosporaceae spp., Diversisporaceae spp., Gigasporaceae spp., Pacisporaceae spp., Funneliformis spp., Glomus spp., Rhizophagus spp., Sclerocystis spp., Septoglomus spp., Claroideoglomus spp., Ambispora spp., Archaeospora spp., Geosiphon pyriformis, Paraglomus spp., other species in the division Glomeromycota, or any combination thereof.

In some embodiments of any of the above embodiments, the LysM receptor is selected from the group of a LysM chitooligosaccharide (CO) receptor, a LysM LCO receptor, or a LysM peptidoglycan (PGN) receptor. In some embodiments, the hydrophobic patch is adjacent to a chitin binding motif if the LysM receptor is the LysM CO receptor or the LysM LCO receptor. In some embodiments, the hydrophobic patch is within 30 Å, 29 Å, 28 Å, 27 Å, 26 Å, 25 Å, 24 Å, 23 Å, 22 Å, 21 Å, 20 Å, 19 Å, 18 Å, 17 Å, 16 Å, 15 Å, 14 Å, 13 Å, 12 Å, 11 Å, 10 Å, 9.5 Å, 9 Å, 8.5 Å, 8 Å, 7.5 Å, 7 Å, 6.5 Å, 6 Å, 5.5 Å, 5 Å, 4.5 Å, 4 Å, 3.5 Å, 3 Å, 2.5 Å, 2 Å, 1.5 Å, or 1 Å of a chitin binding motif if the LysM receptor is the LysM CO receptor or the LysM LCO receptor. In some embodiments, the hydrophobic patch is adjacent to a glycan binding motif if the LysM receptor is the LysM PGN receptor. In some embodiments, the hydrophobic patch is within 30 Å, 29 Å, 28 Å, 27 Å, 26 Å, 25 Å, 24 Å, 23 Å, 22 Å, 21 Å, 20 Å, 19 Å, 18 Å, 17 Å, 16 Å, 15 Å, 14 Å, 13 Å, 12 Å, 11 Å, 10 Å, 9.5 Å, 9 Å, 8.5 Å, 8 Å, 7.5 Å, 7 Å, 6.5 Å, 6 Å, 5.5 Å, 5 Å, 4.5 Å, 4 Å, 3.5 Å, 3 Å, 2.5 Å, 2 Å, 1.5 Å, or 1 Å of a glycan binding motif if the LysM receptor is the LysM PGN receptor. In some embodiments, the LysM receptor is not an exopolysaccharide (EPS) receptor.

In some embodiments of any of the above embodiments, the LysM receptor is a polypeptide having at least 70% sequence identity, at least 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% sequence identity to SEQ ID NO:34 (i.e., Lotus CERK6; BAI79273.1_LjCERK6). In some embodiments of any of the above embodiments, the LysM receptor is a polypeptide having amino acid sequence SEQ ID NO:34 (i.e., Lotus CERK6; BAI79273.1_LjCERK6).

In some aspects, the present disclosure relates to a modified plant LysM receptor including a first LysM1 domain modified to replace at least part of the first LysM1 domain with at least part of a second LysM1 domain. In some embodiments, the first LysM1 domain is modified by substituting a first part of the first LysM1 domain with a third part of a second LysM1 domain and/or by substituting a second part of the first LysM1 domain with a fourth part of the second LysM1 domain. In some embodiments, the first LysM1 domain and the second LysM1 domain have different affinities, selectivities, and/or specificities for oligosaccharides and the modification of the first LysM1 domain alters the affinity, selectivity, and/or specificity to be more like the second LysM1 domain. In some embodiments, the first part and the third part correspond to SEQ ID NO:30 [Lotus CERK6 region II 43-53] or NGSNLTYISEI, SEQ ID NO: 28 [Lotus NFR 1 region II 41-52] or PGVFILQNITTF; and wherein the second part and the fourth part correspond to SEQ ID NO:31 [Lotus CERK6 region IV 74-82] or ASKDSVQAG; SEQ ID NO:29 [Lotus NFR1 region IV 73-81], or LNDINIQSF. In some embodiments, the first LysM1 domain is selected from the group of SEQ ID NO:32 [LysM1 domain Lotus NFR1; LjNFR1/26-95], SEQ ID NO:33 [LysM1 domain Medicago LYK3; MtLYK3/25-95], or NFR1 DLALASYYILPGVFILQNITTFMQSEIVSSNDAITSYNKDKILNDINIQSFORLNIPFP (SEQ ID NO: 55); and the second LysM1 domain is CERK6: ALAQASYYLLNGSNLTYISEIMQSSLLTKPEDIVSYNQDTIASKDSVQAGQRINVPFP (SEQ ID NO:107). In some embodiments, the first part is selected from SEQ ID NO:30 [Lotus CERK6 region II 43-53] or NGSNLTYISEI; the second part is selected from SEQ ID NO: 31 [Lotus CERK6 region IV 74-82] or ASKDSVQAG; the third part is selected from SEQ ID NO: 28 [Lotus NFR 1 region II 41-52] or PGVFILQNITTF; and the fourth part is selected from SEQ ID NO: 29 [Lotus NFR 1 region IV 73-81] or LNDINIQSF. In some embodiments of any of the above embodiments including the first LysM1 domain being modified to replace at least part of the first LysM1 domain with at least part of a second LysM1 domain, the first LysM1 domain is further modified by substituting a fifth part of the first LysM1 domain with a sixth part of a second LysM1 domain. In some embodiments, the first LysM1 domain is SEQ ID NO:115 [LysM1 domain Lotus NFR1; LjNFR1/32-89] or SEQ ID NO: 106 [LysM1 domain Lotus NFR1; LjNFR1/31-89] and the second LysM1 domain is SEQ ID NO:114 [LysM1 domain Medicago LYK3; MtLYK3/31-89] or SEQ ID NO: 105 [LysM1 domain Medicago LYK3; MtLYK3/30-89]. In some embodiments, wherein the fifth part is SEQ ID NO:53 [Lotus NFR1 region III 59-62; LjNFR1/56-92], and wherein the sixth part is SEQ ID NO:46 [Medicago LYK3 region III 57-62; MtLYK3/57-62]. In some embodiments, the first LysM1 domain is modified by substituting a seventh part of the first LysM1 domain, wherein the seventh part spans the first part of the first LysM1 domain, the second part of the first LysM1 domain, and the fifth part of the first LysM1 domain, with an eighth part of the second LysM1 domain, wherein the eighth part spans the third part of the second LysM1 domain, the fourth part of the second LysM1 domain, and the sixth part of the second LysM1 domain. In some embodiments, the seventh part of the first LysM1 domain is SEQ ID NO:51 [Lotus NFR1 regions II-IV 41-82; LjNFR1/41-82], and the eighth part of the second LysM1 domain is SEQ ID NO:113 [Medicago LYK3 regions II-IV 40-82; MtLYK3/40-82] or SEQ ID NO: 104 [Medicago LYK3 regions II-IV 41-82; MtLYK3/41-82]. In some embodiments, the first LysM1 domain is SEQ ID NO:33 [LysM1 domain Medicago LYK3; MtLYK3/31-89] and the second LysM1 domain is SEQ ID NO:32 [LysM1 domain Lotus NFR1; LjNFR1/32-89]. In some embodiments, the fifth part is SEQ ID NO:46 [Medicago LYK3 region III 57-62; MtLYK3/57-62], and the sixth part is SEQ ID NO:53 [Lotus NFR1 region III 59-62; LjNFR1/59-62]. In some embodiments of any of the above embodiments including the first LysM1 domain being SEQ ID NO:33 and the second LysM1 domain being SEQ ID NO:32, the first LysM1 domain is modified by substituting a seventh part of the first LysM1 domain, wherein the seventh part spans the first part of the first LysM1 domain, the second part of the first LysM1 domain, and the fifth part of the first LysM1 domain, with an eighth part of the second LysM1 domain, wherein the eighth part spans the third part of the second LysM1 domain, the fourth part of the second LysM1 domain, and the sixth part of the second LysM1 domain. In some embodiments, the seventh part of the first LysM1 domain is SEQ ID NO:51 [Lotus NFR1 regions II-IV 41-82; LjNFR1/41-82], and the eighth part of the second LysM1 domain is SEQ ID NO:113 [Medicago LYK3 regions II-IV 40-82; MtLYK3/40-82] or SEQ ID NO: 104 [Medicago LYK3 regions II-IV 41-82; MtLYK3/41-82].

In some embodiments of any of the above embodiments including the first LysM1 domain being modified to replace at least part of the first LysM1 domain with at least part of a second LysM1 domain, the entire first LysM1 domain was replaced with the entire second LysM1 domain. In some embodiments of any of the above embodiments including the first LysM1 domain being modified to replace at least part of the first LysM1 domain with at least part of a second LysM1 domain, either or both (i) 80% or fewer, 79% or fewer, 78% or fewer, 77% or fewer, 76% or fewer, 75% or fewer, 74% or fewer, 73% or fewer, 72% or fewer, 71% or fewer, 70% or fewer, 69% or fewer, 68% or fewer, 67% or fewer, 66% or fewer, 65% or fewer, 64% or fewer, 63% or fewer, 62% or fewer, 61% or fewer, 60% or fewer, 59% or fewer, 58% or fewer, 57% or fewer, 56% or fewer, 55% or fewer, 54% or fewer, 53% or fewer, 52% or fewer, 51% or fewer, 50% or fewer, 49% or fewer, 48% or fewer, 47% or fewer, 46% or fewer, 45% or fewer, 44% or fewer, 43% or fewer, 42% or fewer, 41% or fewer, 40% or fewer, 39% or fewer, 38% or fewer, 37% or fewer, 36% or fewer, 35% or fewer, 34% or fewer, 33% or fewer, 32% or fewer, 31% or fewer, 30% or fewer, 29% or fewer, 28% or fewer, 27% or fewer, 26% or fewer, 25% or fewer, 24% or fewer, 23% or fewer, 22% or fewer, 21% or fewer, or 20% or fewer of amino acid residues in the first LysM1 domain were substituted or deleted with the corresponding amino acid residues of the second LysM1 domain, and (ii) the entire LysM1 domain in the unmodified plant LysM receptor was not substituted with another entire LysM2 domain to generate the modified plant LysM receptor. In some embodiments, the modified LysM1 domain binds a lipo-chitooligosaccharide (LCO) produced by nitrogen-fixing bacteria or by mycorrhizal fungi. In some embodiments, the LCO is produced by nitrogen-fixing bacteria selected from the group of Mesorhizobium loti, Mesorhizobium huakuii, Mesorhizobium mediterraneum, Mesorhizobium ciceri, Mesorhizobium spp., Rhizobium mongolense, Rhizobium tropici, Rhizobium etli phaseoli, Rhizobium giardinii, Rhizobium leguminosarum optionally R. leguminosarum trifolii, R. leguminosarum viciae, and R. leguminosarum phaseoli, Burkholderiales optionally symbionts of Mimosa, Sinorhizobium meliloti, Sinorhizobium medicae, Sinorhizobium fredii, Sinorhizobium NGR234, Azorhizobium caulinodans, Bradyrhizobium japonicum, Bradyrhizobium elkanii, Bradyrhizobium liaonginense, Frankia spp., or any combination thereof, or by mycorrhizal fungi selected from the group of Acaulosporaceae spp., Diversisporaceae spp., Gigasporaceae spp., Pacisporaceae spp., Funneliformis spp., Glomus spp., Rhizophagus spp., Sclerocystis spp., Septoglomus spp., Claroideoglomus spp., Ambispora spp., Archaeospora spp., Geosiphon pyriformis, Paraglomus spp., other species in the division Glomeromycota, or any combination thereof. In some embodiments, the modified LysM1 domain binds an LCO with higher affinity than an unmodified LysM1 domain. In some embodiments, the modified LysM1 domain binds LCOs with higher selectivity than an unmodified LysM1 domain. In some embodiments, the modified LysM1 domain binds LCOs with altered specificity as compared to an unmodified LysM1 domain. In some embodiments, structural modelling was used to define the LysM1 domain and was used to identify the first part, the second part, the third part, and/or the fourth part for substitution. In some embodiments, the unmodified plant LysM receptor was selected using the method of any one of the aspects of the present disclosure relating to such selection including any and all embodiments thereof and the second LysM2 domain is from the donor plant LysM receptor. In some embodiments, the receptor of the above embodiments further contains a LysM2 domain modified to contain a hydrophobic patch as in any one of the previous embodiments relating to modifying the LysM2 domain.

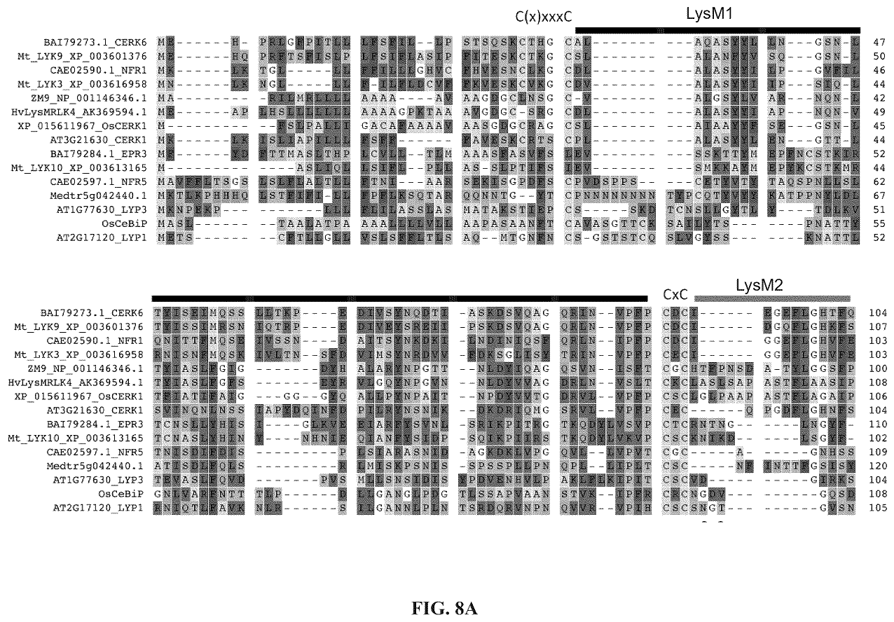

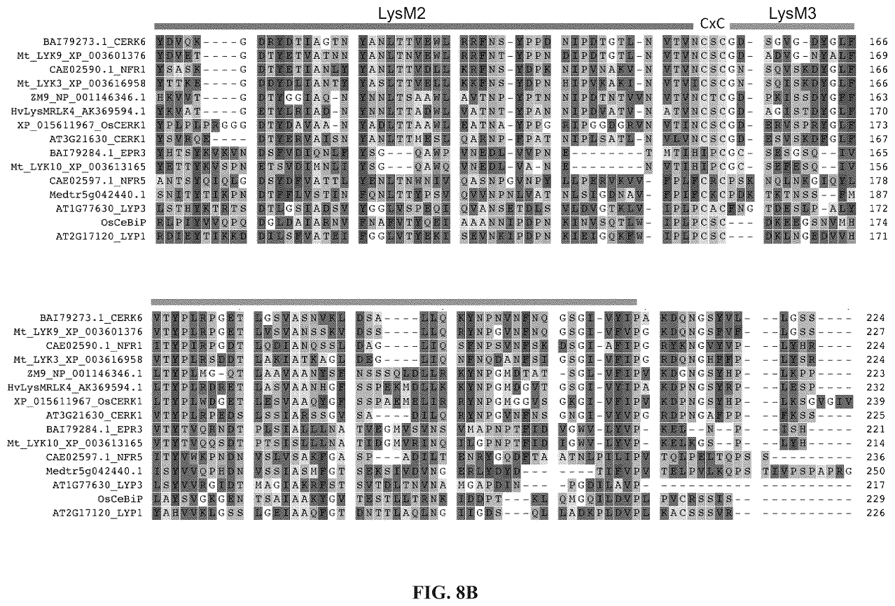

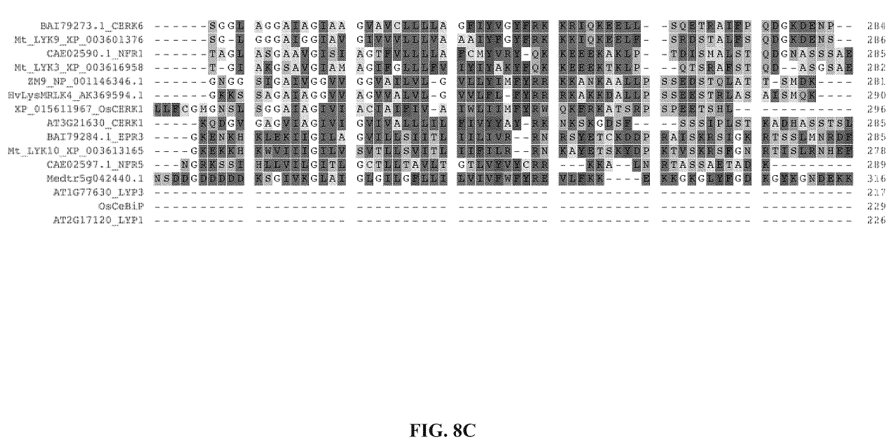

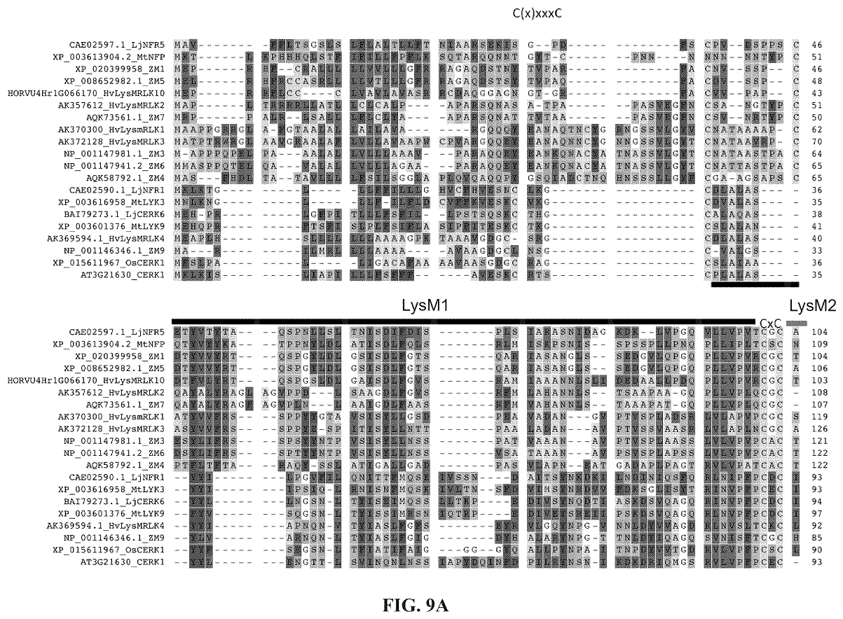

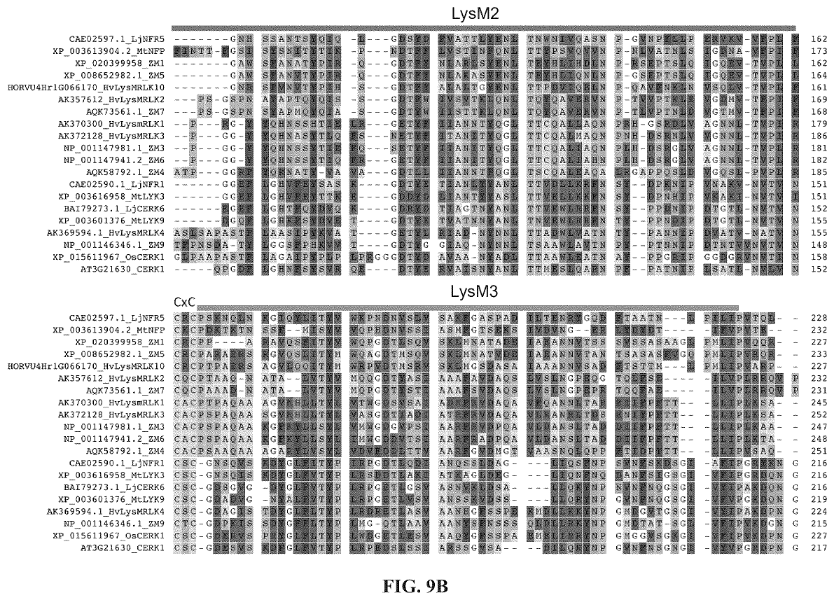

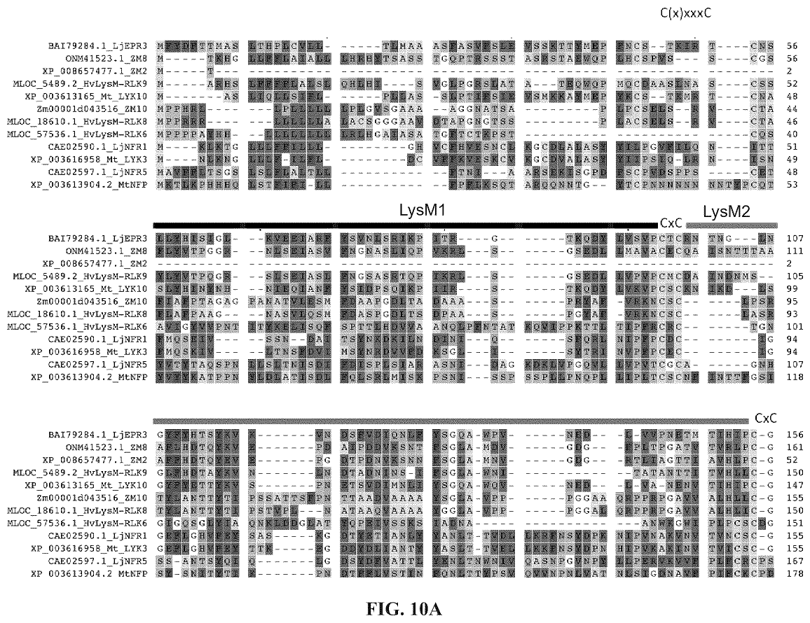

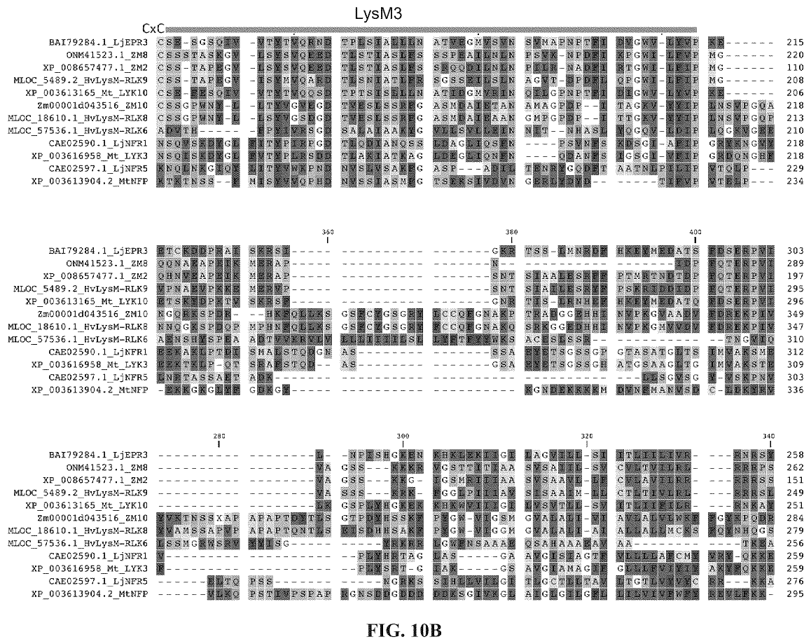

LysM receptors are a well known and well understood type of receptor. LysM receptors have three characteristic domains located in the ectodomain of the protein: LysM1, LysM2, and LysM3, which are present in this order on the protein sequence. The LysM1 domain is located toward the N-terminal end of the protein sequence, and is preceded by an N-terminal signal peptide as well as a C(x)xxxC motif (SEQ ID NO:108). The LysM1 domain is separated from the LysM2 domain by a CxC motif, and the LysM2 domain is separated from the LysM3 domain by a CxC motif as well. The three LysM domains, as well as the C(x)xxxC (SEQ ID NO: 108) and CxC motif are clearly shown in FIGS. 8A-8C, FIGS. 9A-9B, FIGS. 10A-10B, and FIGS. 11A-11B that show individual alignments of Nod factor (e.g., LCO) LysM receptors, EPS LysM receptors, and chitin (CO) as well as PGN LysM receptors, again clearly depicting the three LysM domains as well as the C(x)xxxC (SEQ ID NO: 108) and CxC motifs. The category of LysM receptors is therefore known by one of skill in the art.

As used in the present disclosure, the term “affinity” refers to affinity for LCOs generally. The LysM receptors of the present disclosure may contain a hydrophobic patch in their LysM2 domain. Without wanting to be limited to theory, it is believed that LysM receptors with the hydrophobic patch have higher affinity for LCOs as compared to LysM receptors without the hydrophobic patch, but LysM receptors with domain-swapped LysM1 domains would also provide higher affinity for LCOs and other agonists. Affinity can be measured using the methods described in the Examples below, and using other methods known in the art that measure binding kinetics, association, dissociation, and K_{D}.

As used in the present disclosure, the term “selectivity” refers to the differentiation between different polysaccharide ligands, specifically between lipo-chitooligosaccharides (LCOs) as a class and other polysaccharide ligands, preferably chitooligosaccharides (COs). Without wanting to be limited to theory, it is believed that this hydrophobic patch confers selective recognition of LCOs over COs, and that therefore LysM receptors with the hydrophobic patch have increased selectivity as compared to LysM receptors without the hydrophobic patch. In addition, the LysM receptors with domain-swapped LysM1 domains should also have higher or altered selectivity depend upon the choice of the donor receptor.

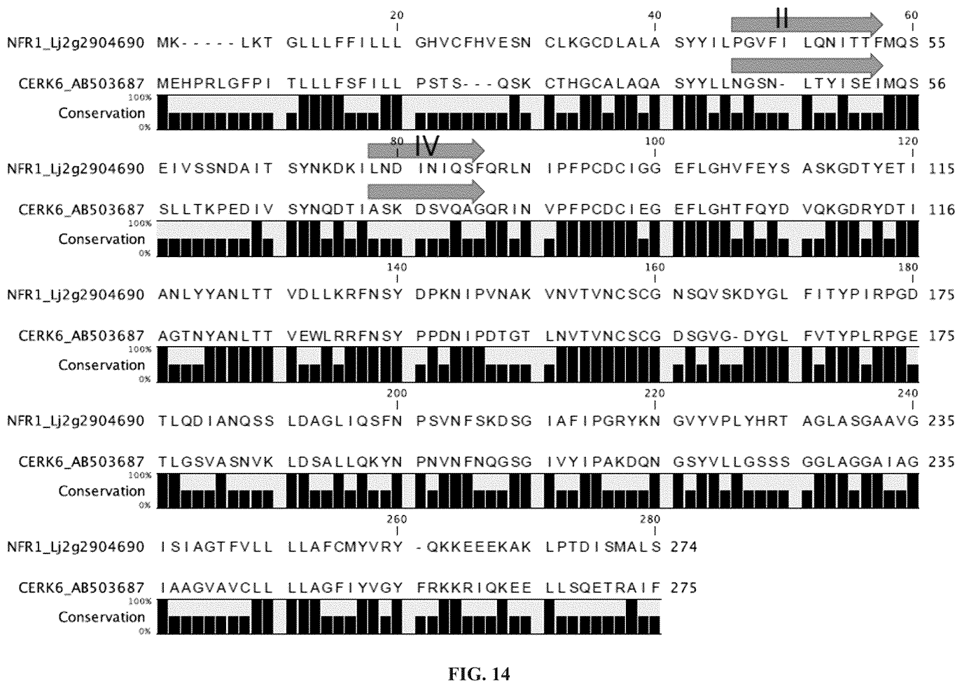

As used in the present disclosure, the term “specificity” refers to the differentiation between different lipo-chitooligosaccharides (LCOs) produced by different nitrogen-fixing bacterial species and/or mycorrhizal fungi. The LysM receptors of the present disclosure may contain a LysM1 domain where regions (e.g., partial, entire) in the LysM1 domain have been replaced with the corresponding regions of the LysM1 domain from a donor LysM receptor. Without wanting to be limited to theory, it is believed that if the donor LysM receptor is a high affinity and specificity LCO LysM receptor such as a legume NFR1 receptor, this replacement can alter the specificity of the LysM receptor, but LysM receptors with a hydrophobic patch in the LysM2 domain may also provide specificity for specific LCOs. The LysM1 domain is clearly shown in FIG. 14, which shows an alignment between Lotus NFR1 and Lotus CERK6, and clearly designates region II and region IV within the LysM1 domain. LysM1 domain replacement can confer highly specific recognition of LCOs produced by particular nitrogen-fixing bacterial species and/or mycorrhizal fungal species, and therefore LysM receptors with the replaced domain can have altered specificity as compared to LysM receptors without the replaced domain, which allows the modified receptors to recognize different nitrogen-fixing bacterial species and/or mycorrhizal fungal species. For at least these reasons, the high affinity, high selectivity, and/or high specificity LysM receptors of the present disclosure will be readily understood by one of skill in the art.

### Genetically Altered Plants and Seeds

In some aspects, the present disclosure relates to a genetically altered plant or part thereof, comprising a nucleic acid sequence encoding a modified plant LysM receptor of any one of the embodiments described in the section “Modified plant LysM receptors”. In some embodiments, the modified plant LysM receptor has higher selectivity and/or affinity for LCOs than the unmodified plant LysM receptor and the expression of the modified plant LysM receptor allows the plant or part thereof to recognize LCOs with high selectivity and/or affinity. In some embodiments, the LCOs are produced by nitrogen-fixing bacteria or by mycorrhizal fungi. In some embodiments, the LCOs are produced by nitrogen-fixing bacteria selected from the group consisting of Mesorhizobium loti, Mesorhizobium huakuii, Mesorhizobium mediterraneum, Mesorhizobium ciceri, Mesorhizobium spp., Rhizobium mongolense, Rhizobium tropici, Rhizobium etli phaseoli, Rhizobium giardinii, Rhizobium leguminosarum optionally R. leguminosarum trifolii, R. leguminosarum viciae, and R. leguminosarum phaseoli, Burkholderiales optionally symbionts of Mimosa, Sinorhizobium meliloti, Sinorhizobium medicae, Sinorhizobium fredii, Sinorhizobium NGR234, Azorhizobium caulinodans, Bradyrhizobium japonicum, Bradyrhizobium elkanii, Bradyrhizobium liaonginense, Frankia spp., and any combination thereof, or by or by mycorrhizal fungi selected from the group consisting of Acaulosporaceae spp., Diversisporaceae spp., Gigasporaceae spp., Pacisporaceae spp., Funneliformis spp., Glomus spp., Rhizophagus spp., Sclerocystis spp., Septoglomus spp., Claroideoglomus spp., Ambispora spp., Archaeospora spp., Geosiphon pyriformis, Paraglomus spp., other species in the division Glomeromycota, and any combination thereof. In some embodiments, the modified polypeptide is localized to a plant cell plasma membrane. In some embodiments, the plant cell is a root cell. In some embodiments, the root cell is a root epidermal cell. In some embodiments, the root cell is a root cortex cell. In some embodiments, the modified polypeptide is expressed in a developing plant root system. In some embodiments, the nucleic acid sequence is operably linked to a promoter. In some embodiments, the promoter is a root specific promoter. In some embodiments, the promoter is selected from the group of a NFR1/LYK3/CERK6 or NFR5/NFP promoter, the Lotus NFR5 promoter (SEQ ID NO:24) and the Lotus NFR1 promoters (SEQ ID NO:25) the maize allothioneine promoter, the chitinase promoter, the maize ZRP2 promoter, the tomato LeExtl promoter, the glutamine synthetase soybean root promoter, the RCC3 promoter, the rice antiquitine promoter, the LRR receptor kinase promoter, or the Arabidopsis pCO2 promoter. In some embodiments, the promoter is a constitutive promoter optionally selected from the group of the CaMV35S promoter, a derivative of the CaMV35S promoter, the maize ubiquitin promoter, the trefoil promoter, a vein mosaic cassava virus promoter, or the Arabidopsis UBQ10 promoter. In some embodiments, the plant is selected from the group of corn (e.g., maize, Zea mays), rice (e.g., Oryza sativa, Oryza glaberrima, Zizania spp.), barley (e.g., Hordeum vulgare), wheat (e.g., common wheat, spelt, durum, Triticum aestivum, Triticum spelta, Triticum durum, Triticum spp.), Trema spp. (e.g., Trema cannabina, Trema cubense, Trema discolor, Trema domingensis, Trema integerrima, Trema lamarckiana, Trema micrantha, Trema orientalis, Trema philippinensis, Trema strigilosa, Trema tomentosa), apple (e.g., Malus pumila), pear (e.g., Pyrus communis, Pyrus x bretschneideri, Pyrus pyrifolia, Pyrus sinkiangensis, Pyrus pashia, Pyrus spp.), plum (e.g., prune, damson, bullaces, Prunus domestica, Prunus salicina), apricot (e.g., Prunus armeniaca, Prunus brigantina, Prunus mandshurica, Prunus mume, Prunus sibirica), peach (e.g., nectarine, Prunus persica), almond (e.g., Prunus dulcis, Prunus amygdalus), walnut (e.g., Persian walnut, English walnut, black walnut, Juglans regia, Juglans nigra, Juglans cinerea, Juglans californica), strawberry (e.g., Fragaria x ananassa, Fragaria chiloensis, Fragaria virginiana, Fragaria vesca), raspberry (e.g., European red raspberry, black raspberry, Rubus idaeus, Rubus occidentalis, Rubus strigosus), blackberry (e.g., evergreen blackberry, Himalayan blackberry, Rubus fruticosus, Rubus ursinus, Rubus laciniatus, Rubus argutus, Rubus armeniacus, Rubus plicatus, Rubus ulmifolius, Rubus allegheniensis), red currant (e.g., Ribes rubrum, Ribes spicatum, Ribesbes alpinum, Ribes schlechtendalii, Ribes multiflorum, Ribes petraeum, Ribes triste), black currant (e.g., Ribes nigrum), melon (e.g., watermelon, winter melon, casabas, cantaloupe, honeydew, muskmelon, Citrullus lanatus, Benincasa hispida, Cucumis melo cantalupensis, Cucumis melo inodorus, Cucumis melo reticulatus), cucumber (e.g., slicing cucumbers, pickling cucumbers, English cucumber, Cucumis sativus), pumpkin (e.g., Cucurbita pepo, Cucurbita maxima), squash (e.g., gourd, Cucurbita argyrosperma, Cucurbita ficifolia, Cucurbita maxima, Cucurbita moschata), grape (e.g., Vitis vinifera, Vitis amurensis, Vitis labrusca, Vitis mustangensis, Vitis riparia, Vitis rotundifolia), bean (e.g., Phaseolus vulgaris, Phaseolus lunatus, Vigna angularis, Vigna radiate, Vigna mungo, Phaseolus coccineus, Vigna umbellate, Vigna acontifolia, Phaseolus acutifolius, Vicia faba, Vicia faba equine, Phaseolus spp., Vigna spp.), soybean (e.g., soy, soya bean, Glycine max, Glycine soja), pea (e.g., Pisum spp., Pisum sativum var. sativum, Pisum sativum var. arvense), chickpea (e.g., garbanzo, Bengal gram, Cicer arietinum), cowpea (e.g., black-eyed pea, blackeye bean, Vigna unguiculata), pigcon pea (e.g., Arhar/Toor, cajan pea, Congo bean, gandules, Caganus cajan), lentil (e.g., Lens culinaris), Bambara groundnut (e.g., earth pea, Vigna subterranea), lupin (e.g., Lupinus spp.), pulses (e.g., minor pulses, Lablab purpureaus, Canavalia ensiformis, Canavalia gladiate, Psophocarpus tetragonolobus, Mucuna pruriens var. utilis, Pachyrhizus erosus), Medicago spp. (e.g., Medicago sativa, Medicago truncatula, Medicago arborea), Lotus spp. (e.g., Lotus japonicus), forage legumes (e.g., Leucaena spp., Albizia spp., Cyamopsis spp., Sesbania spp., Stylosanthes spp., Trifolium spp., Vicia spp.), indigo (e.g., Indigofera spp., Indigofera tinctoria, Indigofera suffruticosa, Indigofera articulata, Indigofera oblongifolia, Indigofera aspalthoides, Indigofera suffruticosa, Indigofera arrecta), legume trees (e.g., locust trees, Gleditsia spp., Robinia spp., Kentucky coffeetree, Gymnocladus dioicus, Acacia spp., Laburnum spp., Wisteria spp.), or hemp (e.g., cannabis, Cannabis sativa).

In some aspects, the present disclosure relates to a genetically altered plant or part thereof, comprising a first nucleic acid sequence encoding a modified plant LysM receptor where the LysM1 domain has been modified as in any of the preceding embodiments relating to modification of the LysM1 domain and a second nucleic acid sequence encoding a modified plant LysM receptor where the LysM2 domain has been modified to include a hydrophobic patch as in any of the preceding embodiments relating to modification of the LysM2 domain. In some embodiments, the modified plant LysM receptor has higher selectivity and/or affinity for LCOs than the unmodified plant LysM receptor and the expression of the modified plant LysM receptor allows the plant or part thereof to recognize LCOs with high selectivity and/or affinity. In some embodiments, the LCOs are produced by nitrogen-fixing bacteria or by mycorrhizal fungi. In some embodiments, the LCOs are produced by nitrogen-fixing bacteria selected from the group consisting of Mesorhizobium loti, Mesorhizobium huakuii, Mesorhizobium mediterraneum, Mesorhizobium ciceri, Mesorhizobium spp., Rhizobium mongolense, Rhizobium tropici, Rhizobium etli phaseoli, Rhizobium giardinii, Rhizobium leguminosarum optionally R. leguminosarum trifolii, R. leguminosarum viciae, and R. leguminosarum phaseoli, Burkholderiales optionally symbionts of Mimosa, Sinorhizobium meliloti, Sinorhizobium medicae, Sinorhizobium fredii, Sinorhizobium NGR234, Azorhizobium caulinodans, Bradyrhizobium japonicum, Bradyrhizobium elkanii, Bradyrhizobium liaonginense, Frankia spp., and any combination thereof, or by or by mycorrhizal fungi selected from the group consisting of Acaulosporaceae spp., Diversisporaceae spp., Gigasporaceae spp., Pacisporaceae spp., Funneliformis spp., Glomus spp., Rhizophagus spp., Sclerocystis spp., Septoglomus spp., Claroideoglomus spp., Ambispora spp., Archaeospora spp., Geosiphon pyriformis, Paraglomus spp., other species in the division Glomeromycota, and any combination thereof. In some embodiments, the modified polypeptide is localized to a plant cell plasma membrane. In some embodiments, the plant cell is a root cell. In some embodiments, the root cell is a root epidermal cell. In some embodiments, the root cell is a root cortex cell. In some embodiments, the modified polypeptide is expressed in a developing plant root system. In some embodiments, the first nucleic acid or second nucleic acid sequence is operably linked to a promoter. In some embodiments, the promoter is a root specific promoter. In some embodiments, the promoter is selected from the group of a NFR1/LYK3/CERK6 or NFR5/NFP promoter, the Lotus NFR5 promoter (SEQ ID NO:24) and the Lotus NFR 1 promoters (SEQ ID NO:25) the maize allothioneine promoter, the chitinase promoter, the maize ZRP2 promoter, the tomato LeExtl promoter, the glutamine synthetase soybean root promoter, the RCC3 promoter, the rice antiquitine promoter, the LRR receptor kinase promoter, or the Arabidopsis pCO2 promoter. In some embodiments, the promoter is a constitutive promoter optionally selected from the group of the CaMV35S promoter, a derivative of the CaMV35S promoter, the maize ubiquitin promoter, the trefoil promoter, a vein mosaic cassava virus promoter, or the Arabidopsis UBQ10 promoter. In some embodiments, the plant is selected from the group of corn (e.g., maize, Zea mays), rice (e.g., Oryza sativa, Oryza glaberrima, Zizania spp.), barley (e.g., Hordeum vulgare), wheat (e.g., common wheat, spelt, durum, Triticum aestivum, Triticum spelta, Triticum durum, Triticum spp.), Trema spp. (e.g., Trema cannabina, Trema cubense, Trema discolor, Trema domingensis, Trema integerrima, Trema lamarckiana, Trema micrantha, Trema orientalis, Trema philippinensis, Trema strigilosa, Trema tomentosa), apple (e.g., Malus pumila), pear (e.g., Pyrus communis, Pyrus x bretschneideri, Pyrus pyrifolia, Pyrus sinkiangensis, Pyrus pashia, Pyrus spp.), plum (e.g., prune, damson, bullaces, Prunus domestica, Prunus salicina), apricot (e.g., Prunus armeniaca, Prunus brigantina, Prunus mandshurica, Prunus mume, Prunus sibirica), peach (e.g., nectarine, Prunus persica), almond (e.g., Prunus dulcis, Prunus amygdalus), walnut (e.g., Persian walnut, English walnut, black walnut, Juglans regia, Juglans nigra, Juglans cinerea, Juglans californica), strawberry (e.g., Fragaria x ananassa, Fragaria chiloensis, Fragaria virginiana, Fragaria vesca), raspberry (e.g., European red raspberry, black raspberry, Rubus idaeus, Rubus occidentalis, Rubus strigosus), blackberry (e.g., evergreen blackberry, Himalayan blackberry, Rubus fruticosus, Rubus ursinus, Rubus laciniatus, Rubus argutus, Rubus armeniacus, Rubus plicatus, Rubus ulmifolius, Rubus allegheniensis), red currant (e.g., Ribes rubrum, Ribes spicatum, Ribesbes alpinum, Ribes schlechtendalii, Ribes multiflorum, Ribes petraeum, Ribes triste), black currant (e.g., Ribes nigrum), melon (e.g., watermelon, winter melon, casabas, cantaloupe, honeydew, muskmelon, Citrullus lanatus, Benincasa hispida, Cucumis melo cantalupensis, Cucumis melo inodorus, Cucumis melo reticulatus), cucumber (e.g., slicing cucumbers, pickling cucumbers, English cucumber, Cucumis sativus), pumpkin (e.g., Cucurbita pepo, Cucurbita maxima), squash (e.g., gourd, Cucurbita argyrosperma, Cucurbita ficifolia, Cucurbita maxima, Cucurbita moschata), grape (e.g., Vitis vinifera, Vitis amurensis, Vitis labrusca, Vitis mustangensis, Vitis riparia, Vitis rotundifolia), bean (e.g., Phaseolus vulgaris, Phaseolus lunatus, Vigna angularis, Vigna radiate, Vigna mungo, Phaseolus coccineus, Vigna umbellata, Vigna acontifolia, Phaseolus acutifolius, Vicia faba, Vicia faba equine, Phaseolus spp., Vigna spp.), soybean (e.g., soy, soya bean, Glycine max, Glycine soja), pea (e.g., Pisum spp., Pisum sativum var. sativum, Pisum sativum var. arvense), chickpea (e.g., garbanzo, Bengal gram, Cicer arietinum), cowpea (e.g., black-eyed pea, blackeye bean, Vigna unguiculata), pigeon pea (e.g., Arhar/Toor, cajan pea, Congo bean, gandules, Caganus cajan), lentil (e.g., Lens culinaris), Bambara groundnut (e.g., earth pea, Vigna subterranea), lupin (e.g., Lupinus spp.), pulses (e.g., minor pulses, Lablab purpureaus, Canavalia ensiformis, Canavalia gladiate, Psophocarpus tetragonolobus, Mucuna pruriens var. utilis, Pachyrhizus erosus), Medicago spp. (e.g., Medicago sativa, Medicago truncatula, Medicago arborea), Lotus spp. (e.g., Lotus japonicus), forage legumes (e.g., Leucaena spp., Albizia spp., Cyamopsis spp., Sesbania spp., Stylosanthes spp., Trifolium spp., Vicia spp.), indigo (e.g., Indigofera spp., Indigofera tinctoria, Indigofera suffruticosa, Indigofera articulata, Indigofera oblongifolia, Indigofera aspalthoides, Indigofera suffruticosa, Indigofera arrecta), legume trees (e.g., locust trees, Gleditsia spp., Robinia spp., Kentucky coffeetree, Gymnocladus dioicus, Acacia spp., Laburnum spp., Wisteria spp.), or hemp (e.g., cannabis, Cannabis sativa). In some embodiments, the plant part is a leaf, a stem, a root, a root primordia, a flower, a seed, a fruit, a kernel, a grain, a cell, or a portion thereof. In some embodiments, the plant part is a fruit, a kernel, or a grain.

In some aspects, the present disclosure relates to a pollen grain or an ovule of a genetically altered plant of any of the above embodiments relating to plants.

In some aspects, the present disclosure relates to a protoplast from a genetically altered plant of any of the above embodiments relating to plants.

In some aspects, the present disclosure relates to a tissue culture produced from protoplasts or cells from a genetically altered plant of any of the above embodiments relating to plants, wherein the cells or protoplasts are produced from a plant part selected from the group consisting of leaf, anther, pistil, stem, petiole, root, root primordia, root tip, fruit, seed, flower, cotyledon, hypocotyl, embryo, and meristematic cell.

### Methods of Producing and Cultivating Genetically Altered Plants

Certain aspects of the present disclosure relate to a method of producing the genetically altered plant of any one of the above embodiments relating to plants as described in the section “Genetically altered plants and seeds”, comprising introducing a genetic alteration to the plant comprising the nucleic acid sequence. In some embodiments, the nucleic acid sequence, the first nucleic acid sequence, and/or the second nucleic acid sequence is operably linked to a promoter. In some embodiments, the promoter is a root specific promoter. In some embodiments, the promoter is selected from the group of a NFR1/LYK3/CERK6 or NFR5/NFP promoter, the Lotus NFR5 promoter (SEQ ID NO:24) and the Lotus NFR1 promoters (SEQ ID NO:25) the maize allothioneine promoter, the chitinase promoter, the maize ZRP2 promoter, the tomato LeExtl promoter, the glutamine synthetase soybean root promoter, the RCC3 promoter, the rice antiquitine promoter, the LRR receptor kinase promoter, or the Arabidopsis pCO2 promoter. In some embodiments, the promoter is a constitutive promoter optionally selected from the group of the CaMV35S promoter (KAY et al. Science, 236, 4805, 1987), a derivative of the CaMV35S promoter, the maize ubiquitin promoter, the trefoil promoter, a vein mosaic cassava virus promoter, or the Arabidopsis UBQ10 promoter. In some embodiments, the nucleic acid sequence, the first nucleic acid sequence, and/or the second nucleic acid sequence is inserted into the genome of the plant so that the nucleic acid sequence, the first nucleic acid sequence, and/or the second nucleic acid sequence is operably linked to an endogenous promoter. In some embodiments, the endogenous promoter is a root specific promoter.

In some aspects, the present disclosure relates to a method of producing a genetically altered plant able to recognize LCOs, comprising the steps of: introducing a genetic alteration to the plant comprising the provision of an ability for LCOs produced by nitrogen-fixing bacteria and/or mycorrhizal fungi to be recognized, thereby enabling the plant to recognize LCOs.

In some aspects, the present disclosure relates to a method of producing a genetically altered plant able to recognize LCOs, comprising the steps of: introducing a genetic alteration to the plant comprising the provision of an ability for LCOs produced by nitrogen-fixing bacteria and/or mycorrhizal fungi to be recognized with high affinity, high selectivity, and/or high specificity, thereby enabling the plant to recognize LCOs with high affinity, high selectivity, and/or high specificity.

In some aspects, the present disclosure relates to a method of producing a genetically altered plant able to recognize LCOs produced by a specific nitrogen-fixing bacterial species and/or a specific mycorrhizal fungal species, comprising the steps of: introducing a genetic alteration to the plant comprising the provision of an ability for LCOs produced by the specific nitrogen-fixing bacterial species and/or the specific mycorrhizal fungal species to be recognized with altered specificity, thereby enabling the plant to recognize LCOs with altered specificity. In some embodiments, the genetic alteration allows the genetically altered plant to recognize a different specific nitrogen-fixing bacterial species and/or specific mycorrhizal fungal species as compared to a plant without the genetic alteration. In some embodiments, the genetically altered plant is able to be grown in different agricultural conditions (e.g., different soils containing different symbiotic microbial species, etc.). In some embodiments, the genetic alteration allows the genetically altered plant to be grown in different agricultural conditions containing specific bacterial strains producing LCOs detected with high specificity, sensitivity, and/or selectivity by the genetically altered plant. In some embodiments, the bacterial strains are added as a seed coating or as a soil inoculum. In some embodiments, the genetically altered plant is able to be grown with different crop species (e.g., different crop rotations, etc.).

In some aspects, the present disclosure relates to a method of cultivating a plant with the ability to recognize LCOs, comprising the steps of: providing a seed with one or more genetic alterations that provide an ability for LCOs produced by nitrogen-fixing bacteria and/or mycorrhizal fungi to be recognized, wherein the seed produces a plant with the ability to recognize LCOs produced by nitrogen-fixing bacteria and/or mycorrhizal fungi; cultivating the plant under conditions where the ability to recognize LCOs produced by nitrogen-fixing bacteria and/or mycorrhizal fungi results in increased growth, yield, and/or biomass, as compared to a plant grown under the same conditions that lacks the one or more genetic alterations. In some embodiments, the plant is cultivated in nutrient-poor soil.

In some aspects, the present disclosure relates to a method of cultivating a plant with the ability to recognize LCOs with high affinity, high selectivity, and/or high specificity, comprising the steps of: providing a seed with one or more genetic alterations that provide an ability for LCOs produced by nitrogen-fixing bacteria and/or mycorrhizal fungi to be recognized with high affinity, high selectivity, and/or high specificity, wherein the seed produces a plant with the ability to recognize LCOs produced by nitrogen-fixing bacteria and/or mycorrhizal fungi with high affinity, high selectivity, and/or high specificity; cultivating the plant under conditions where the ability to recognize LCOs produced by nitrogen-fixing bacteria and/or mycorrhizal fungi with high affinity, high selectivity, and/or high specificity results in increased growth, yield, and/or biomass, as compared to a plant grown under the same conditions that lacks the one or more genetic alterations. In some embodiments, the plant is cultivated in nutrient-poor soil.

In some aspects, the present disclosure relates to a method of cultivating a plant with the ability to recognize LCOs with altered specificity, comprising the steps of: providing a seed with one or more genetic alterations that provide an ability for LCOs produced by nitrogen-fixing bacteria and/or mycorrhizal fungi to be recognized with altered specificity, wherein the seed produces a plant with the ability to recognize LCOs produced by nitrogen-fixing bacteria and/or mycorrhizal fungi with high specificity; cultivating the plant under conditions where the ability to recognize LCOs produced by nitrogen-fixing bacteria and/or mycorrhizal fungi with altered specificity results in increased growth, yield, and/or biomass, as compared to a plant grown under the same conditions that lacks the one or more genetic alterations. In some embodiments, the plant is cultivated in nutrient-poor soil. In some embodiments, the genetic alteration allows the genetically altered plant to recognize a different specific nitrogen-fixing bacterial species and/or specific mycorrhizal fungal species as compared to a plant without the genetic alteration. In some embodiments, the genetically altered plant is able to be grown in different agricultural conditions (e.g., different soils containing different symbiotic microbial species, etc.). In some embodiments, the genetically altered plant is able to be grown with different crop species (e.g., different crop rotations, etc.).

In some aspects, the present disclosure relates to a method of cultivating a plant with the ability to recognize LCOs, comprising the steps of: providing a tissue culture or protoplast with one or more genetic alterations that provide an ability for LCOs produced by nitrogen-fixing bacteria and/or mycorrhizal fungi to be recognized; regenerating the tissue culture or protoplast into a plantlet; growing the plantlet into a plant, wherein the plant has the ability to recognize LCOs produced by nitrogen-fixing bacteria and/or mycorrhizal fungi; transplanting the plant into conditions where the ability to recognize LCOs produced by nitrogen-fixing bacteria and/or mycorrhizal fungi results in increased growth, yield, and/or biomass, as compared to a plant grown under the same conditions that lacks the one or more genetic alterations. In some embodiments, the plant is cultivated in nutrient-poor soil.

In some aspects, the present disclosure relates to a method of cultivating a plant with the ability to recognize LCOs with high affinity, high selectivity, and/or high specificity, comprising the steps of: providing a tissue culture or protoplast with one or more genetic alterations that provide an ability for LCOs produced by nitrogen-fixing bacteria and/or mycorrhizal fungi to be recognized with high affinity, high selectivity, and/or high specificity, regenerating the tissue culture or protoplast into a plantlet; growing the plantlet into a plant, wherein the plant has the ability to recognize LCOs produced by produced by nitrogen-fixing bacteria and/or mycorrhizal fungi with high affinity, high selectivity, and/or high specificity; transplanting the plant into conditions where the ability to recognize LCOs produced by nitrogen-fixing bacteria and/or mycorrhizal fungi with high affinity, high selectivity, and/or high specificity results in increased growth, yield, and/or biomass, as compared to a plant grown under the same conditions that lacks the one or more genetic alterations. In some embodiments, the plant is cultivated in nutrient-poor soil.

In some aspects, the present disclosure relates to a method of cultivating a plant with the ability to recognize LCOs with altered specificity, comprising the steps of: providing a tissue culture or protoplast with one or more genetic alterations that provide an ability for LCOs produced by nitrogen-fixing bacteria and/or mycorrhizal fungi to be recognized with altered specificity, regenerating the tissue culture or protoplast into a plantlet; growing the plantlet into a plant, wherein the plant has the ability to recognize LCOs produced by produced by nitrogen-fixing bacteria and/or mycorrhizal fungi with high specificity; transplanting the plant into conditions where the ability to recognize LCOs produced by nitrogen-fixing bacteria and/or mycorrhizal fungi with altered specificity results in increased growth, yield, and/or biomass, as compared to a plant grown under the same conditions that lacks the one or more genetic alterations. In some embodiments, the plant is cultivated in nutrient-poor soil. In some embodiments, the genetic alteration allows the genetically altered plant to recognize a different specific nitrogen-fixing bacterial species and/or specific mycorrhizal fungal species as compared to a plant without the genetic alteration. In some embodiments, the genetically altered plant is able to be grown in different agricultural conditions (e.g., different soils containing different symbiotic microbial species, etc.). In some embodiments, the genetically altered plant is able to be grown with different crop species (e.g., different crop rotations, etc.).

In some embodiments of any of the above methods, the ability to recognize LCOs is conferred by a nucleic acid sequence encoding a modified plant LysM receptor of any one of the embodiments described in the section “Modified plant LysM receptors”. In some embodiments, the modified plant LysM receptor has higher selectivity and/or affinity for LCOs than the unmodified plant LysM receptor and the expression of the modified plant LysM receptor allows the plant or part thereof to recognize LCOs with high selectivity and/or affinity. In some embodiments, the LCOs are produced by nitrogen-fixing bacteria or by mycorrhizal fungi. In some embodiments, the LCOs are produced by nitrogen-fixing bacteria selected from the group consisting of Mesorhizobium loti, Mesorhizobium huakuii, Mesorhizobium mediterraneum, Mesorhizobium ciceri, Mesorhizobium spp., Rhizobium mongolense, Rhizobium tropici, Rhizobium etli phaseoli, Rhizobium giardinii, Rhizobium leguminosarum optionally R. leguminosarum trifolii, R. leguminosarum viciae, and R. leguminosarum phaseoli, Burkholderiales optionally symbionts of Mimosa, Sinorhizobium meliloti, Sinorhizobium medicae, Sinorhizobium fredii, Sinorhizobium NGR234, Azorhizobium caulinodans, Bradyrhizobium japonicum, Bradyrhizobium elkanii, Bradyrhizobium liaonginense, Frankia spp., and any combination thereof, or by or by mycorrhizal fungi selected from the group consisting of Acaulosporaceae spp., Diversisporaceae spp., Gigasporaceae spp., Pacisporaceae spp., Funneliformis spp., Glomus spp., Rhizophagus spp., Sclerocystis spp., Septoglomus spp., Claroideoglomus spp., Ambispora spp., Archaeospora spp., Geosiphon pyriformis, Paraglomus spp., other species in the division Glomeromycota, and any combination thereof. In some embodiments, the modified polypeptide is localized to a plant cell plasma membrane. In some embodiments, the plant cell is a root cell. In some embodiments, the root cell is a root epidermal cell. In some embodiments, the root cell is a root cortex cell. In some embodiments, the modified polypeptide is expressed in a developing plant root system. In some embodiments, the nucleic acid sequence is operably linked to a promoter. In some embodiments, the promoter is a root specific promoter. In some embodiments, the promoter is selected from the group of a NFR1/LYK3/CERK6 or NFR5/NFP promoter, the Lotus NFR5 promoter (SEQ ID NO:24) and the Lotus NFR1 promoters (SEQ ID NO:25) the maize allothioneine promoter, the chitinase promoter, the maize ZRP2 promoter, the tomato LeExtl promoter, the glutamine synthetase soybean root promoter, the RCC3 promoter, the rice antiquitine promoter, the LRR receptor kinase promoter, or the Arabidopsis pCO2 promoter. In some embodiments, the promoter is a constitutive promoter optionally selected from the group of the CaMV35S promoter, a derivative of the CaMV35S promoter, the maize ubiquitin promoter, the trefoil promoter, a vein mosaic cassava virus promoter, or the Arabidopsis UBQ10 promoter. In some embodiments, the plant is selected from the group of corn (e.g., maize, Zea mays), rice (e.g., Oryza sativa, Oryza glaberrima, Zizania spp.), barley (e.g., Hordeum vulgare), wheat (e.g., common wheat, spelt, durum, Triticum aestivum, Triticum spelta, Triticum durum, Triticum spp.), Trema spp. (e.g., Trema cannabina, Trema cubense, Trema discolor, Trema domingensis, Trema integerrima, Trema lamarckiana, Trema micrantha, Trema orientalis, Trema philippinensis, Trema strigilosa, Trema tomentosa), apple (e.g., Malus pumila), pear (e.g., Pyrus communis, Pyrus xbretschneideri, Pyrus pyrifolia, Pyrus sinkiangensis, Pyrus pashia, Pyrus spp.), plum (e.g., prune, damson, bullaces, Prunus domestica, Prunus salicina), apricot (e.g., Prunus armeniaca, Prunus brigantina, Prunus mandshurica, Prunus mume, Prunus sibirica), peach (e.g., nectarine, Prunus persica), almond (e.g., Prunus dulcis, Prunus amygdalus), walnut (e.g., Persian walnut, English walnut, black walnut, Juglans regia, Juglans nigra, Juglans cinerea, Juglans californica), strawberry (e.g., Fragaria x ananassa, Fragaria chiloensis, Fragaria virginiana, Fragaria vesca), raspberry (e.g., European red raspberry, black raspberry, Rubus idaeus, Rubus occidentalis, Rubus strigosus), blackberry (e.g., evergreen blackberry, Himalayan blackberry, Rubus fruticosus, Rubus ursinus, Rubus laciniatus, Rubus argutus, Rubus armeniacus, Rubus plicatus, Rubus ulmifolius, Rubus allegheniensis), red currant (e.g., Ribes rubrum, Ribes spicatum, Ribesbes alpinum, Ribes schlechtendalii, Ribes multiflorum, Ribes petraeum, Ribes triste), black currant (e.g., Ribes nigrum), melon (e.g., watermelon, winter melon, casabas, cantaloupe, honeydew, muskmelon, Citrullus lanatus, Benincasa hispida, Cucumis melo cantalupensis, Cucumis melo inodorus, Cucumis melo reticulatus), cucumber (e.g., slicing cucumbers, pickling cucumbers, English cucumber, Cucumis sativus), pumpkin (e.g., Cucurbita pepo, Cucurbita maxima), squash (e.g., gourd, Cucurbita argyrosperma, Cucurbita ficifolia, Cucurbita maxima, Cucurbita moschata), grape (e.g., Vitis vinifera, Vitis amurensis, Vitis labrusca, Vitis mustangensis, Vitis riparia, Vitis rotundifolia), bean (e.g., Phaseolus vulgaris, Phaseolus lunatus, Vigna angularis, Vigna radiate, Vigna mungo, Phaseolus coccineus, Vigna umbellate, Vigna acontifolia, Phaseolus acutifolius, Vicia faba, Vicia faba equine, Phaseolus spp., Vigna spp.), soybean (e.g., soy, soya bean, Glycine max, Glycine soja), pea (e.g., Pisum spp., Pisum sativum var. sativum, Pisum sativum var. arvense), chickpea (e.g., garbanzo, Bengal gram, Cicer arietinum), cowpea (e.g., black-eyed pea, blackeye bean, Vigna unguiculata), pigcon pea (e.g., Arhar/Toor, cajan pea, Congo bean, gandules, Caganus cajan), lentil (e.g., Lens culinaris), Bambara groundnut (e.g., earth pea, Vigna subterranea), lupin (e.g., Lupinus spp.), pulses (e.g., minor pulses, Lablab purpureaus, Canavalia ensiformis, Canavalia gladiate, Psophocarpus tetragonolobus, Mucuna pruriens var. utilis, Pachyrhizus erosus), Medicago spp. (e.g., Medicago sativa, Medicago truncatula, Medicago arborea), Lotus spp. (e.g., Lotus japonicus), forage legumes (e.g., Leucaena spp., Albizia spp., Cyamopsis spp., Sesbania spp., Stylosanthes spp., Trifolium spp., Vicia spp.), indigo (e.g., Indigofera spp., Indigofera tinctoria, Indigofera suffruticosa, Indigofera articulata, Indigofera oblongifolia, Indigofera aspalthoides, Indigofera suffruticosa, Indigofera arrecta), legume trees (e.g., locust trees, Gleditsia spp., Robinia spp., Kentucky coffeetree, Gymnocladus dioicus, Acacia spp., Laburnum spp., Wisteria spp.), or hemp (e.g., cannabis, Cannabis sativa).

### Molecular Biological Methods to Produce Genetically Altered Plants and Plant Cells

One embodiment of the present invention provides a genetically altered plant or plant cell comprising one or more modified endogenous plant genes. For example, the present disclosure provides plants with genetically altered LysM receptors modified to include a hydrophobic patch or alter the hydrophobic patch in the LysM2 domain and plants with genetically altered LysM receptors modified by replacing regions in the LysM1 domain with corresponding donor LysM1 domain regions. Plants with these modified receptors can have increased affinity, selectivity, and/or specificity for LCOs.

Certain aspects of the present disclosure relate to methods for selection of a target plant LysM receptor for modifying the target plant LysM receptor to have a desired receptor characteristic, wherein the method includes the steps of: a) providing a structural model, a molecular model, a surface characteristics model, and/or an electrostatic potential model of a donor plant LysM receptor having the desired receptor characteristic and two or more potential target plant LysM receptors; b) comparing each of the two or more potential target plant LysM receptors with the structural model, the molecular model, the surface characteristics model, and/or the electrostatic potential model of the donor plant LysM receptor, and/or comparing each of the two or more potential target plant LysM receptors with the donor plant LysM receptor using structural overlay; and c) selecting the potential target plant LysM receptor with a suitable match for the donor plant LysM receptor to be the target plant LysM receptor. In some embodiments, the criteria for determining that the potential target plant LysM receptor is a suitable match for the donor plant LysM receptor in step (c) are selected from the group of goodness of fit to template structure; similarity; phylogenetic relation; surface potential; coverage to template structure; GMQE, QMEAN, and Local Quality estimates from SWISS-Model; or any combination thereof. In some embodiments, the structural model of a donor plant LysM receptor is a protein crystal structure, a molecular model, a cryo-EM structure, and a NMR structure. In some embodiments, the donor plant LysM receptor model is of an entire ectodomain and the two or more potential target plant LysM receptor models are of entire ectodomains. In some embodiments, the donor plant LysM receptor model is of a LysM1 domain, a LysM2 domain, a LysM3 domain, or any combination thereof, and the two or more potential target plant LysM receptor models are of LysM1 domains, LysM2 domains, LysM3 domains, or any combination thereof.

In some embodiments, the donor plant LysM receptor is Medicago NFP, Medicago LYK3, Lotus NFR1, Lotus NFR5, Lotus LYS11, or Arabidopsis CERK1. In some embodiments, the two or more target plant LysM receptors are additionally compared to Lotus CERK6. In some embodiments, the two or more potential target plant LysM receptor polypeptides are all from the same plant species or plant variety. In some embodiments, the desired receptor characteristic is affinity, selectivity, and/or specificity for an oligosaccharide or class of oligosaccharides. In some embodiments, the desired receptor characteristic is binding kinetics for an oligosaccharide or class of oligosaccharides, wherein the binding kinetics include off-rate and on-rate. In some embodiments, the class of oligosaccharides is selected from the group of LCOs, COs, beta-glucans, cyclic-beta-glucans, exopolysaccharides, or optionally LPS. In some embodiments, the class of oligosaccharides is LCOs or COs. In some embodiments, the class of oligosaccharides is LCOs, optionally produced by a produced by a nitrogen-fixing bacteria optionally selected from the group of Mesorhizobium loti, Mesorhizobium huakuii, Mesorhizobium mediterraneum, Mesorhizobium ciceri, Mesorhizobium spp., Rhizobium mongolense, Rhizobium tropici, Rhizobium etli phaseoli, Rhizobium giardinii, Rhizobium leguminosarum optionally R. leguminosarum trifolii, R. leguminosarum viciae, and R. leguminosarum phaseoli, Burkholderiales optionally symbionts of Mimosa, Sinorhizobium meliloti, Sinorhizobium medicae, Sinorhizobium fredii, Sinorhizobium NGR234, Azorhizobium caulinodans, Bradyrhizobium japonicum, Bradyrhizobium elkanii, Bradyrhizobium liaonginense, Frankia spp., or any combination thereof; or optionally produced by a mycorrhizal fungi optionally selected from the group of Acaulosporaceae spp., Diversisporaceae spp., Gigasporaceae spp., Pacisporaceae spp., Funneliformis spp., Glomus spp., Rhizophagus spp., Sclerocystis spp., Septoglomus spp., Claroideoglomus spp., Ambispora spp., Archaeospora spp., Geosiphon pyriformis, Paraglomus spp., other species in the division Glomeromycota, pr any combination thereof. In some embodiments, the LCOs are M. loti LCO, S. meliloti LCO-IV, or S. meliloti LCO-V.

In some embodiments, the method further includes step d) identifying one or more amino acid residues for modification in the target LysM receptor by comparing amino acid residues of a first oligosaccharide binding feature in the donor plant LysM receptor with the corresponding amino acid residues in the target plant LysM receptor, and optionally identifying one or more amino acid residues for modification in the target LysM receptor by comparing amino acid residues of a second oligosaccharide binding feature in the donor plant LysM receptor with the corresponding amino acid residues in the target plant LysM receptor. In some embodiments, the method further includes step e) generating a modified plant LysM receptor wherein the one or more amino acid residues in the first oligosaccharide binding feature of the target plant LysM receptor have been substituted with corresponding amino acid residues from the donor LysM; generating a modified plant LysM receptor wherein the one or more amino acid residues in the second oligosaccharide binding feature of the target plant LysM receptor have been substituted with corresponding amino acid residues from the donor LysM; or generating a modified plant LysM receptor wherein the one or more amino acid residues in the first oligosaccharide binding feature of the target plant LysM receptor have been substituted with corresponding amino acid residues from the donor LysM and the one or more amino acid residues in the second oligosaccharide binding feature of the target plant LysM receptor have been substituted with corresponding amino acid residues from the donor LysM. In some embodiments, the first oligosaccharide binding feature is a hydrophobic patch on the surface of the LysM2 domain. In some embodiments, the second oligosaccharide binding feature is a part of the LysM1 domain of the donor plant LysM receptor. In some embodiments, the modified LysM receptor is an endogenous LysM receptor modified using one or more gene editing components. In some embodiments, the one or more gene editing components target a nuclear genome sequence operably linked to a nucleic acid encoding an endogenous LysM receptor (e.g., a soybean LysM receptor). A further embodiment of this aspect includes the one or more gene editing components being selected from the group of a ribonucleoprotein complex that targets the nuclear genome sequence; a vector comprising a TALEN protein encoding sequence, wherein the TALEN protein targets the nuclear genome sequence; a vector comprising a ZFN protein encoding sequence, wherein the ZEN protein targets the nuclear genome sequence; an oligonucleotide donor (ODN), wherein the ODN targets the nuclear genome sequence; or a vector comprising a CRISPR/Cas enzyme encoding sequence and a targeting sequence, wherein the targeting sequence targets the nuclear genome sequence.

Additional aspects of the present disclosure relate to a modified plant LysM receptor produced using any one of the above methods, wherein the modified plant LysM receptor includes a LysM2 domain modified to comprise a hydrophobic patch on the surface of the LysM2 domain. Further aspects of the present disclosure relate to a modified plant LysM receptor produced using any one of the above methods, wherein the modified plant LysM receptor includes a first LysM1 domain modified to replace at least part of the first LysM1 domain with at least part of a second LysM1 domain. The modified plant LysM receptors of the present disclosure may be used to produce the genetically altered plant of any one of the above embodiments relating to plants as described in the section “Genetically altered plants and seeds”

Transformation and generation of genetically altered monocotyledonous and dicotyledonous plant cells is well known in the art. Sec, e.g., Weising, et al., Ann. Rev. Genet. 22:421-477 (1988); U.S. Pat. No. 5,679,558; Agrobacterium Protocols, ed: Gartland, Humana Press Inc. (1995); and Wang, et al. Acta Hort. 461:401-408 (1998). The choice of method varies with the type of plant to be transformed, the particular application and/or the desired result. The appropriate transformation technique is readily chosen by the skilled practitioner.

Any methodology known in the art to delete, insert or otherwise modify the cellular DNA (e.g., genomic DNA and organelle DNA) can be used in practicing the inventions disclosed herein. For example, a disarmed Ti plasmid, containing a genetic construct for deletion or insertion of a target gene, in Agrobacterium tumefaciens can be used to transform a plant cell, and thereafter, a transformed plant can be regenerated from the transformed plant cell using procedures described in the art, for example, in EP 0116718, EP 0270822, PCT publication WO 84/02913 and published European Patent application (“EP”) 0242246. Ti-plasmid vectors each contain the gene between the border sequences, or at least located to the left of the right border sequence, of the T-DNA of the Ti-plasmid. Of course, other types of vectors can be used to transform the plant cell, using procedures such as direct gene transfer (as described, for example in EP 0233247), pollen mediated transformation (as described, for example in EP 0270356, PCT publication WO 85/01856, and U.S. Pat. No. 4,684,611), plant RNA virus-mediated transformation (as described, for example in EP 0 067 553 and U.S. Pat. No. 4,407,956), liposome-mediated transformation (as described, for example in U.S. Pat. No. 4,536,475), and other methods such as the methods for transforming certain lines of corn (e.g., U.S. Pat. No. 6,140,553; Fromm et al., Bio/Technology (1990) 8, 833 839); Gordon-Kamm et al., The Plant Cell, (1990) 2, 603 618) and rice (Shimamoto et al., Nature, (1989) 338, 274 276; Datta et al., Bio/Technology, (1990) 8, 736 740) and the method for transforming monocots generally (PCT publication WO 92/09696). For cotton transformation, the method described in PCT patent publication WO 00/71733 can be used. For soybean transformation, reference is made to methods known in the art, e.g., Hinchee et al. (Bio/Technology, (1988) 6, 915) and Christou et al. (Trends Biotech, (1990) 8, 145) or the method of WO 00/42207.

Genetically altered plants of the present invention can be used in a conventional plant breeding scheme to produce more genetically altered plants with the same characteristics, or to introduce the genetic alteration(s) in other varieties of the same or related plant species. Seeds, which are obtained from the altered plants, preferably contain the genetic alteration(s) as a stable insert in chromosomal or organelle DNA or as modifications to an endogenous gene or promoter. Plants comprising the genetic alteration(s) in accordance with the invention include plants comprising, or derived from, root stocks of plants comprising the genetic alteration(s) of the invention, e.g., fruit trees or ornamental plants. Hence, any non-transgenic grafted plant parts inserted on a transformed plant or plant part are included in the invention.

Introduced genetic elements, whether in an expression vector or expression cassette, which result in the expression of an introduced gene will typically utilize a plant-expressible promoter. A ‘plant-expressible promoter’ as used herein refers to a promoter that ensures expression of the genetic alteration(s) of the invention in a plant cell. Examples of promoters directing constitutive expression in plants are known in the art and include: the strong constitutive 35S promoters (the “35S promoters”) of the cauliflower mosaic virus (CaMV), e.g., of isolates CM 1841 (Gardner et al., Nucleic Acids Res, (1981) 9, 2871 2887), CabbB S (Franck et al., Cell (1980) 21, 285 294) and CabbB JI (Hull and Howell, Virology, (1987) 86, 482 493); promoters from the ubiquitin family (e.g., the maize ubiquitin promoter of Christensen et al., Plant Mol Biol, (1992) 18, 675-689), the gos2 promoter (de Pater et al., The Plant J (1992) 2, 834-844), the emu promoter (Last et al., Theor Appl Genet, (1990) 81, 581-588), actin promoters such as the promoter described by An et al. (The Plant J, (1996) 10, 107), the rice actin promoter described by Zhang et al. (The Plant Cell, (1991) 3, 1155-1165); promoters of the Cassava vein mosaic virus (WO 97/48819, Verdaguer et al. (Plant Mol Biol, (1998) 37, 1055-1067), the pPLEX series of promoters from Subterranean Clover Stunt Virus (WO 96/06932, particularly the S4 or S7 promoter), an alcohol dehydrogenase promoter, e.g., pAdh1S (GenBank accession numbers X04049, X00581), and the TR1′ promoter and the TR2′ promoter (the “TR1′ promoter” and “TR2′ promoter”, respectively) which drive the expression of the l′ and 2′ genes, respectively, of the T DNA (Velten et al., EMBO J, (1984) 3, 2723 2730).

Alternatively, a plant-expressible promoter can be a tissue-specific promoter, i.e., a promoter directing a higher level of expression in some cells or tissues of the plant, e.g., in root epidermal cells or root cortex cells. Examples of constitutive promoters that are often used in plant cells are the cauliflower mosaic (CaMV) 35S promoter (KAY et al. Science, 236, 4805, 1987), and various derivatives of the promoter, virus promoter vein mosaic cassava (International Application WO 97/48819), the maize ubiquitin promoter (CHRISTENSEN & QUAIL, Transgenic Res, 5, 213-8, 1996), trefoil (Ljubql, MAEKAWA et al. Mol Plant Microbe Interact. 21, 375-82, 2008) and Arabidopsis (UBQ10, Norris et al. Plant Mol. Biol. 21, 895-906, 1993).

In preferred embodiments, root specific promoters will be used. Non-limiting examples include the promoter of the maize allothioneine (DE FRAMOND et al, FEBS 290, 103.-106, 1991 Application EP 452269), the chitinase promoter (SAMAC et al. Plant Physiol 93, 907-914, 1990), the glutamine synthetase soybean root promoter (HIREL et al. Plant Mol. Biol. 20, 207-218, 1992), the RCC3 promoter (PCT Application WO 2009/016104), the rice antiquitine promoter (PCT Application WO 2007/076115), the LRR receptor kinase promoter (PCT application WO 02/46439), the maize ZRP2 promoter (U.S. Pat. No. 5,633,363), the tomato LExtl promoter (Bucher et al. Plant Physiol. 128, 911-923, 2002), and the Arabidopsis pCO2 promoter (HEIDSTRA et al, Genes Dev. 18, 1964-1969, 2004). These plant promoters can be combined with enhancer elements, they can be combined with minimal promoter elements, or can comprise repeated elements to ensure the expression profile desired.

In some embodiments, genetic elements to increase expression in plant cells can be utilized. For example, an intron at the 5′ end or 3′ end of an introduced gene, or in the coding sequence of the introduced gene, e.g., the hsp70 intron. Other such genetic elements can include, but are not limited to, promoter enhancer elements, duplicated or triplicated promoter regions, 5′ leader sequences different from another transgene or different from an endogenous (plant host) gene leader sequence, 3′ trailer sequences different from another transgene used in the same plant or different from an endogenous (plant host) trailer sequence.

An introduced gene of the present invention can be inserted in host cell DNA so that the inserted gene part is upstream (i.e., 5′) of suitable 3′ end transcription regulation signals (e.g., transcript formation and polyadenylation signals). This is preferably accomplished by inserting the gene in the plant cell genome (nuclear or chloroplast). Preferred polyadenylation and transcript formation signals include those of the nopaline synthase gene (Depicker et al., J. Molec Appl Gen, (1982) 1, 561-573), the octopine synthase gene (Gielen et al., EMBO J, (1984) 3:835 845), the SCSV or the Malic enzyme terminators (Schunmann et al., Plant Funct Biol, (2003) 30:453-460), and the T DNA gene 7 (Velten and Schell, Nucleic Acids Res, (1985) 13, 6981 6998), which act as 3′ untranslated DNA sequences in transformed plant cells. In some embodiments, one or more of the introduced genes are stably integrated into the nuclear genome. Stable integration is present when the nucleic acid sequence remains integrated into the nuclear genome and continues to be expressed (e.g., detectable mRNA transcript or protein is produced) throughout subsequent plant generations. Stable integration into and/or editing of the nuclear genome can be accomplished by any known method in the art (e.g., microparticle bombardment, Agrobacterium-mediated transformation, CRISPR/Cas9, electroporation of protoplasts, microinjection, etc.).

The term recombinant or modified nucleic acids refers to polynucleotides which are made by the combination of two otherwise separated segments of sequence accomplished by the artificial manipulation of isolated segments of polynucleotides by genetic engineering techniques or by chemical synthesis. In so doing one may join together polynucleotide segments of desired functions to generate a desired combination of functions.

As used herein, the terms “overexpression” and “upregulation” refer to increased expression (e.g., of mRNA, polypeptides, etc.) relative to expression in a wild type organism (e.g., plant) as a result of genetic modification. In some embodiments, the increase in expression is a slight increase of about 10% more than expression in wild type. In some embodiments, the increase in expression is an increase of 50% or more (e.g., 60%, 70%, 80%, 100%, etc.) relative to expression in wild type. In some embodiments, an endogenous gene is overexpressed. In some embodiments, an exogenous gene is overexpressed by virtue of being expressed. Overexpression of a gene in plants can be achieved through any known method in the art, including but not limited to, the use of constitutive promoters, inducible promoters, high expression promoters (e.g., PsaD promoter), enhancers, transcriptional and/or translational regulatory sequences, codon optimization, modified transcription factors, and/or mutant or modified genes that control expression of the gene to be overexpressed.

Where a recombinant nucleic acid is intended for expression, cloning, or replication of a particular sequence, DNA constructs prepared for introduction into a host cell will typically comprise a replication system (e.g. vector) recognized by the host, including the intended DNA fragment encoding a desired polypeptide, and can also include transcription and translational initiation regulatory sequences operably linked to the polypeptide-encoding segment. Additionally, such constructs can include cellular localization signals (e.g., plasma membrane localization signals). In preferred embodiments, such DNA constructs are introduced into a host cell's genomic DNA, chloroplast DNA or mitochondrial DNA.

In some embodiments, a non-integrated expression system can be used to induce expression of one or more introduced genes. Expression systems (expression vectors) can include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Signal peptides can also be included where appropriate from secreted polypeptides of the same or related species, which allow the protein to cross and/or lodge in cell membranes, cell wall, or be secreted from the cell.

Selectable markers useful in practicing the methodologies of the invention disclosed herein can be positive selectable markers. Typically, positive selection refers to the case in which a genetically altered cell can survive in the presence of a toxic substance only if the recombinant polynucleotide of interest is present within the cell. Negative selectable markers and screenable markers are also well known in the art and are contemplated by the present invention. One of skill in the art will recognize that any relevant markers available can be utilized in practicing the inventions disclosed herein.

Screening and molecular analysis of recombinant strains of the present invention can be performed utilizing nucleic acid hybridization techniques. Hybridization procedures are useful for identifying polynucleotides, such as those modified using the techniques described herein, with sufficient homology to the subject regulatory sequences to be useful as taught herein. The particular hybridization techniques are not essential to the subject invention. As improvements are made in hybridization techniques, they can be readily applied by one of skill in the art. Hybridization probes can be labeled with any appropriate label known to those of skill in the art. Hybridization conditions and washing conditions, for example temperature and salt concentration, can be altered to change the stringency of the detection threshold. Sec, e.g., Sambrook et al. (1989) vide infra or Ausubel et al. (1995) Current Protocols in Molecular Biology, John Wiley & Sons, NY, N.Y., for further guidance on hybridization conditions.

Additionally, screening and molecular analysis of genetically altered strains, as well as creation of desired isolated nucleic acids can be performed using Polymerase Chain Reaction (PCR). PCR is a repetitive, enzymatic, primed synthesis of a nucleic acid sequence. This procedure is well known and commonly used by those skilled in this art (see Mullis, U.S. Pat. Nos. 4,683,195, 4,683,202, and 4,800,159; Saiki et al. (1985) Science 230:1350-1354). PCR is based on the enzymatic amplification of a DNA fragment of interest that is flanked by two oligonucleotide primers that hybridize to opposite strands of the target sequence. The primers are oriented with the 3′ ends pointing towards each other. Repeated cycles of heat denaturation of the template, annealing of the primers to their complementary sequences, and extension of the annealed primers with a DNA polymerase result in the amplification of the segment defined by the 5′ ends of the PCR primers. Because the extension product of each primer can serve as a template for the other primer, each cycle essentially doubles the amount of DNA template produced in the previous cycle. This results in the exponential accumulation of the specific target fragment, up to several million-fold in a few hours. By using a thermostable DNA polymerase such as the Taq polymerase, which is isolated from the thermophilic bacterium Thermus aquaticus, the amplification process can be completely automated. Other enzymes which can be used are known to those skilled in the art.

Nucleic acids and proteins of the present invention can also encompass homologues of the specifically disclosed sequences. Homology (e.g., sequence identity) can be 50%-100%. In some instances, such homology is greater than 80%, greater than 85%, greater than 90%, or greater than 95%. The degree of homology or identity needed for any intended use of the sequence(s) is readily identified by one of skill in the art. As used herein percent sequence identity of two nucleic acids is determined using an algorithm known in the art, such as that disclosed by Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:402-410. BLAST nucleotide searches are performed with the NBLAST program, score=100, wordlength=12, to obtain nucleotide sequences with the desired percent sequence identity. To obtain gapped alignments for comparison purposes, Gapped BLAST is used as described in Altschul et al. (1997) Nucl. Acids. Res. 25:3389-3402. When utilizing BLAST and Gapped BLAST programs, the default parameters of the respective programs (NBLAST and XBLAST) are used. See www.ncbi.nih.gov.

Preferred host cells are plant cells. Recombinant host cells, in the present context, are those which have been genetically modified to contain an isolated nucleic molecule, contain one or more deleted or otherwise non-functional genes normally present and functional in the host cell, or contain one or more genes to produce at least one recombinant protein. The nucleic acid(s) encoding the protein(s) of the present invention can be introduced by any means known to the art which is appropriate for the particular type of cell, including without limitation, transformation, lipofection, electroporation or any other methodology known by those skilled in the art.

Having generally described this invention, the same will be better understood by reference to certain specific examples, which are included herein to further illustrate the invention and are not intended to limit the scope of the invention as defined by the claims.

### EXAMPLES

The present disclosure is described in further detail in the following examples which are not in any way intended to limit the scope of the disclosure as claimed. The attached figures are meant to be considered as integral parts of the specification and description of the disclosure. The following examples are offered to illustrate, but not to limit the claimed disclosure.

### Example 1: Structural Characterization of Medicago NFP Ectodomain

The following example describes the structural characterization of the Medicago NFP protein ectodomain.

### Materials and Methods

Expression and purification of Medicago NFP ectodomain: The Medicago truncatula NFP ectodomain (residues 28-246) was codon-optimized for insect cell expression (Genscript, Piscataway, USA) and cloned into the POET4 baculovirus transfer vector (Oxford Expression Technologies). The native NFP signal peptide (residues 1-27, predicted by SignalP 4.1) was replaced with the AcMNPV gp67 signal peptide to facilitate secretion and a hexa-histidine tag was added to the C-terminus. Recombinant baculoviruses were produced in Sf9 cells (Spodoptera frugiperda) using the FlashBac Gold kit (Oxford Expression technologies) according to the manufacturer's instructions with Lipofectin (ThermoFisher Scientific) as a transfection reagent. Protein expression was performed as follows. Suspension-cultured Sf9 cells were maintained with shaking at 299 K in serum-free MAX-XP (BD-Biosciences, discontinued) or HyClone SFX (GE Healthcare) medium supplemented with 1% Pen-Strep (10000 U/ml, Life technologies) and 1% CD lipid concentrate (Gibco). Protein expression was induced by adding recombinant passage 3 virus once the Sf9 cells reached a cell density of 1.0*10\hat{ }6 cells/ml. After 5-7 days of expression, medium supernatant containing NFP ectodomains was harvested by centrifugation. This was followed by an overnight dialysis step against 50 mM Tris-HCl pH 8, 200 mM NaCl at 277 K. The NFP ectodomain was enriched by two subsequent steps of Ni-IMAC purification (HisTrap excel/HisTrap HP, both GE Healthcare). For crystallography, N-glycans were removed using the endoglycosidase PNGase F (1:15 (w/w), room temperature, overnight). As a final purification step, NFP ectodomains was purified by SEC on a Superdex 200 10/300 or HiLoad Superdex 200 16/600 (both GE Healthcare) in phosphate buffered saline at pH 7.2 supplemented to a total of 500 mM NaCl (for binding assays) or 50 mM Tris-HCl, 200 mM NaCl (for crystallography). NFP ectodomain elutes as a single, homogeneous peak corresponding to a monomer.

Crystallization and structure determination: Crystals of deglycosylated NFP ectodomain were obtained using a vapour diffusion setup at 3-5 mg/ml in 0.2 M Na-acetate, 0.1 M Na-cacodylate pH 6.5, and 30% (w/v) PEG-8000. Crystals were cryoprotected in their crystallization condition by supplementing with 5% (w/v) PEG-400 before being snap-frozen in liquid nitrogen. Complete diffraction data to 2.85 Å resolution was obtained at the MaxLab 1911-3 beamline. The phase problem was solved by molecular replacement using Phaser from the PHENIX suite with a homology model based on the AtCERK1 ectodomain structure (PDB coordinates 4EBZ) as a search model. Model building and refinement was done using COOT and the PHENIX suite, respectively. The output pdb filled structural model was generated and its electrostatic surface potential was calculated using the PDB2PQR and APBS webservers (PMID: 21425296). The results were visualized in PyMol using APBS tools 2.1 (DeLano, W. L. (2002). PyMOL. DeLano Scientific, San Carlos, CA, 700.).

### Results

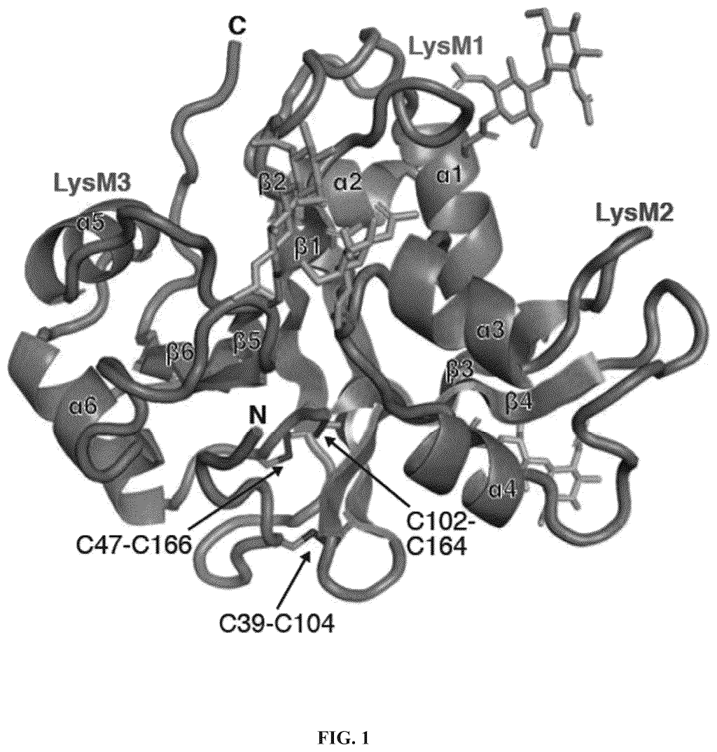

The structure of Medicago NFP was determined by molecular replacement using a homology model based on the inner low B-factor scaffold of AtCERK1. The complete structure of NFP (residues 33-233) was built this way, including four N-glycosylations that were clearly resolved in the 2.8 Å electron density map. NFP forms a compact structure where three classical βααβ LysM domains are tightly interconnected and stabilized by 3 conserved disulfide bridges (C3-C104, C47-C166 and C102-C164) (FIG. 1). The disulfide connectivity pattern and the overall scaffold arrangement is shared with other LysM-RLK proteins involved in chitin defense signaling, supporting a common evolutionary origin of these class of receptors.

### Example 2: Identification of Important Residues for Lipo-Chitooligosaccharide (LCO) Perception

The following example describes the use of a structurally-guided approach to identify important residues in NFP for LCO perception. After identifying important residues, NFP point mutations were created, and tested using ligand-binding assays.

### Materials and Methods

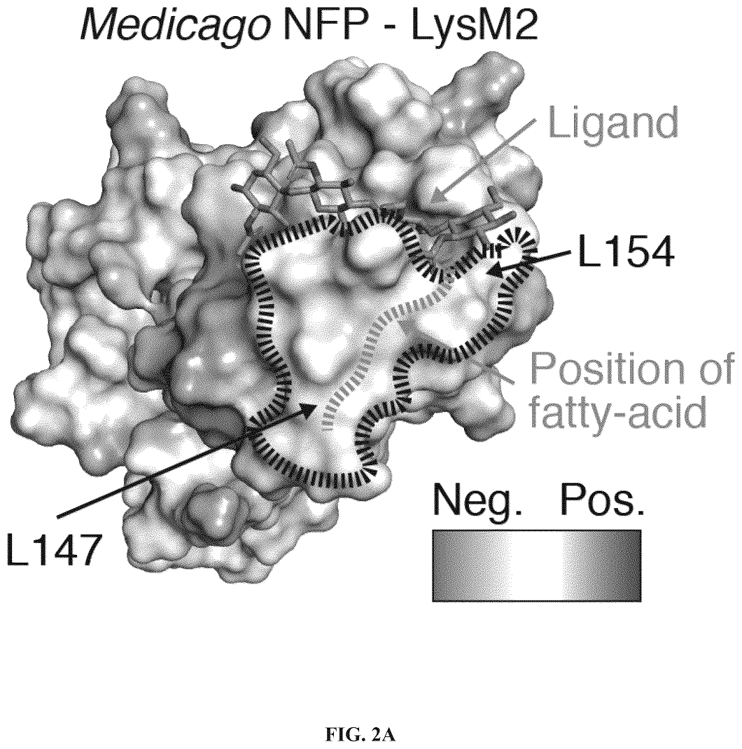

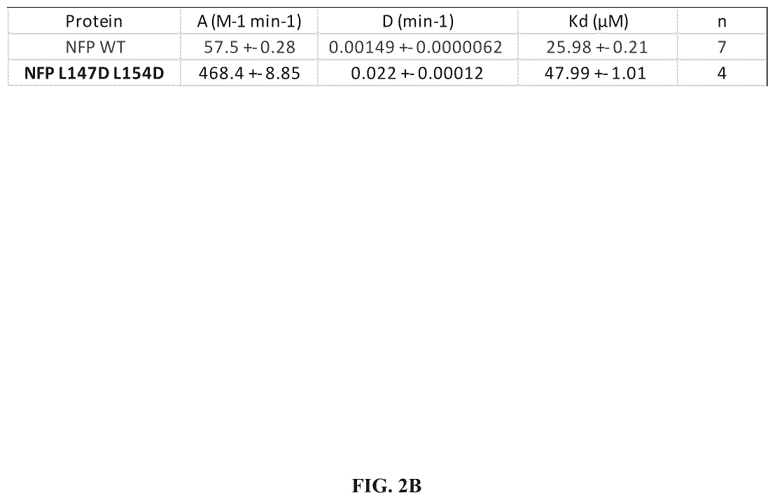

Structurally-guided residue identification: The NFP ectodomain was structurally aligned to ligand-bound CERK1. Then, the electrostatic surface potential was mapped to the previously-developed structure of the NFP ectodomain. The predicted ligand-binding location and electrostatic surface potential are depicted in FIG. 2A.

Creation of NFP point mutations: The NFP leucine residues L147 and L154 were replaced with aspartate residues. Aspartate is similar in size to leucine, but negatively charged where leucine is hydrophobic. Point mutants of NFP were engineered using site-directed mutagenesis. In particular, a double-mutated NFP was engineered where the leucine residues L147 and L154 were replaced with aspartate residues to create the mutant NFP L147D L154D. Point mutated versions of NFP were expressed and purified as described in Example 1.

NFP mutant binding assays: The binding assay using NFP wild type (WT) protein was replicated seven times, while the binding assay using the NFP mutant NFP L147D L154D was replicated four times. A summary of results is shown in FIG. 2B.

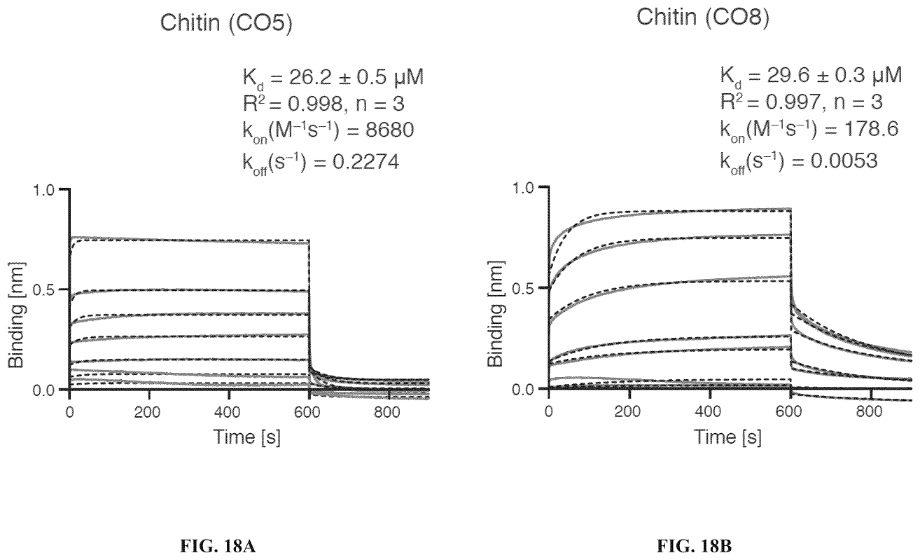

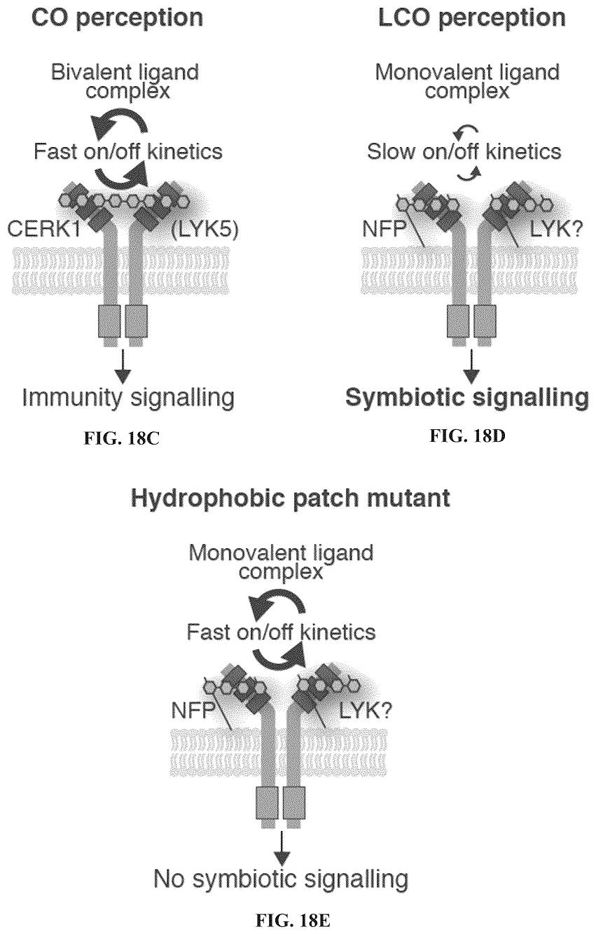

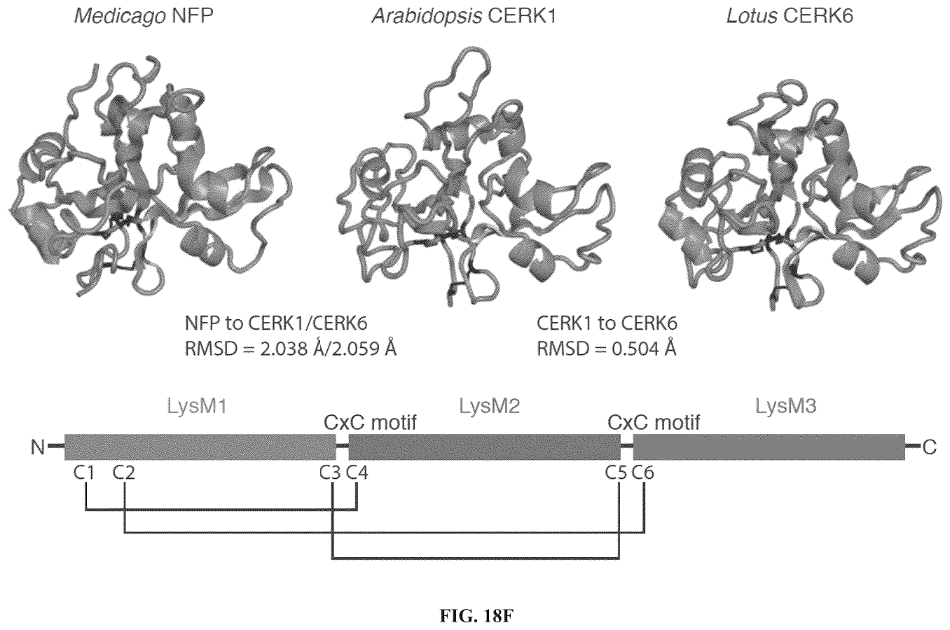

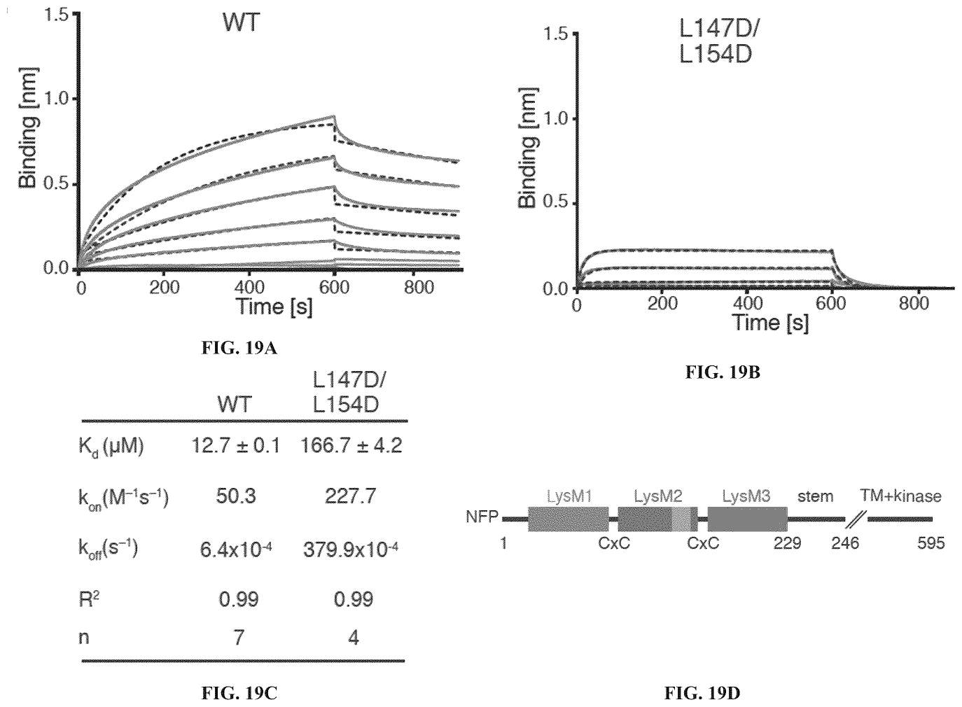

Biolayer interferometry (BLI): Binding of NFP WT and NFP L147D/L154D mutant to S. meliloti LCO-IV was measured on an Octet RED 96 system (Pall ForteBio). S. meliloti LCO consists of a tetrameric/pentameric N-acetylglucosamine backbone that is O-sulfated on the reducing terminal residue, O-acetylated on the non-reducing terminal residue, and mono-N-acylated by unsaturated C16 acyl groups. Biotinylated ligand conjugates were immobilized on streptavidin biosensors (kinetic quality, Pall ForteBio) at a concentration of 125-250 nM for 5 minutes. The binding assays were replicated 7 times for the NFP WT, and 4 times for the NFP L147D/L154D mutant. Data analysis was performed in GraphPad Prism 6 software (GraphPad Software, Inc.). Equilibrium dissociation constants derived from the steady-state were determined by applying a non-linear regression (one site, specific binding) to the response at equilibrium plotted against the protein concentration. Kinetic parameters were determined by non-linear regression (association followed by dissociation) on the subtracted data. Results are shown in FIGS. 19A-19C. Binding of A. thaliana CERK1 (ArCERK1) to chitopentaose (CO5) and chitooctaose (CO8) was measured in the same way. Results are shown in FIGS. 18A-18B.

### Results

FIG. 2A shows modelling of the NFP ectodomain bound to a ligand with predicted chitin and LCO fatty acid chain locations. Structural alignment of the NFP ectodomain with ligand-bound CERK1 positions chitin in the LysM2 binding groove of NFP without any obvious clashes. Strikingly, the electrostatic surface potential revealed a hydrophobic patch on the NFP ectodomain that is located near the non-reducing moiety of the docked chitin molecule, which potentially could accommodate the fatty acid chain of the LCO ligand. Two leucine residues (L147 and L154) were identified as the residues that give this patch its hydrophobic character.

To test the contribution of these two residues to LCO binding, both residues were replaced with similarly sized but negatively charged aspartate residues to produce NFP L147D L154D. Interestingly, the double mutated NFP L147D L154D ectodomain bound S. meliloti LCO-IV with approximately two times lower affinity; Kd of 48.0±1.0 μM (FIG. 2B). Closer inspection of the binding kinetics revealed that the association (Kₒₙ) was almost unaffected whereas the dissociation (K_{off}) was approximately 15 times faster in the double mutant. These results show that the hydrophobic patch of the NFP ectodomain is stabilizing the LCO bound state, and that this stabilization is most likely occurring via the fatty acid chain. Docking the LCO fatty acid in this hydrophobic patch and the chitin backbone in the LysM2 binding site (derived from CERK1) would place the sulphate and acetyl side groups facing K141.

Biochemical analysis of LCO binding to the hydrophobic patch mutant reveals that purified L147D/L154D NFP-ECD bound S. meliloti LCO-IV with 13-fold lower affinity (Kd of 166.7±4.2 μM) compared to WT NFP-ECD (FIGS. 19A-19C). The association rate (kₒₙ) was 4.5-fold faster and the dissociation rate (k_{off}) was dramatically increased with 59-fold in the double mutant compared to the WT NFP-ECD, suggesting that the hydrophobic patch had a strong stabilizing effect on LCO binding mediated by the acyl chain.

The binding kinetics of ArCERK1 binding to chitin fragments were measured as a comparison. As shown in FIGS. 18A-18B, fast association and dissociation rates were seen. These kinetics were reminiscent of the kinetics observed for the mutant L147D/L154D NFP-ECD (FIG. 19B). The binding kinetics of ArCERK1 to chitin fragments were clearly different than the binding kinetics of NFP to LCO (FIG. 19A).

Together, the data provided evidence that the hydrophobic patch in NFP (shown in FIG. 19D) was a conserved structural imprint critical for LCO perception and symbiotic signaling.

### Example 3: Complementation Test in Medicago Nfp Mutants

To confirm the biochemical observations described in the previous examples, next a complementation test was performed in Medicago nfp mutants using hairy root transformation.

### Materials and Methods

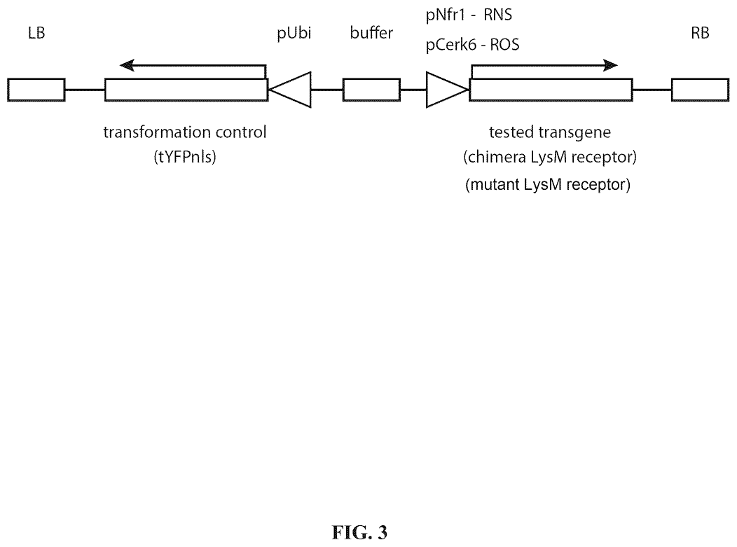

Complementation assay: Construct assembly, plant growth conditions, hairy root transformations, nodulation and ROS assays were generally conducted as described in Bozsoki et al. (2017) (Bozsoki Z, Cheng J, Feng F, Gysel K, Vinther M, Andersen K R, Oldroyd G, Blaise M, Radutoiu S, Stougaard J (2017) Receptor-mediated chitin perception in legume roots is functionally separable from Nod factor perception. Proc Natl Acad Sci 114: E8118-E8127). A general schematic of the construct is provided in FIG. 3. The tested transgenes were the mutated LysM receptors described in Example 2.

### Results

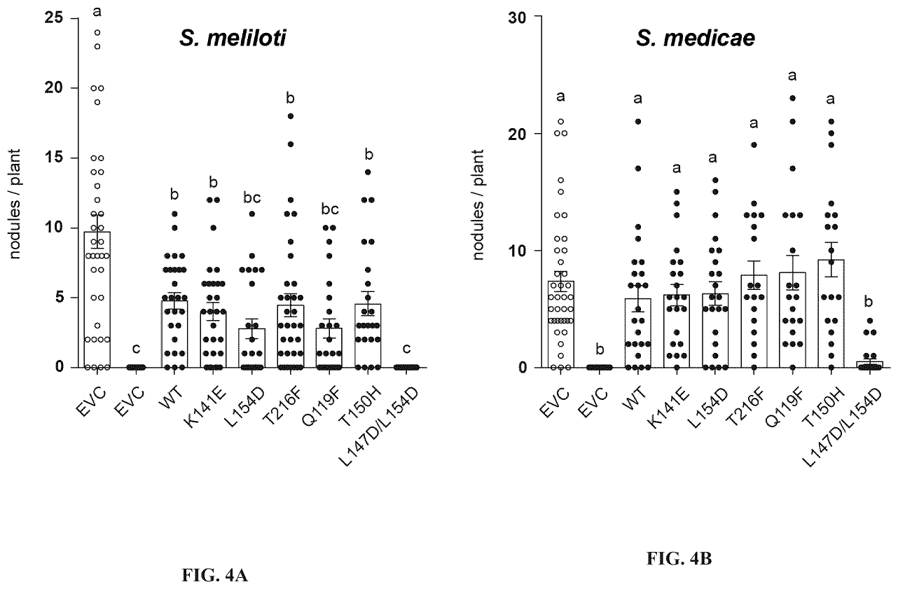

FIGS. 4A-4B shows the results of the complementation test. The results shown in FIG. 4A are complementation tests where the plants were inoculated with S. meliloti strain 2011. When Medicago nfp mutants are transformed with the wild type Nfp gene, complementation is seen, which is defined as an average of 10 nodules per plant 6-7 weeks after inoculation with S. meliloti strain 2011. In contrast, roots transformed with the double mutant construct (L147D/L154D) did not develop any nodules per plant 6-7 weeks after inoculation with S. meliloti strain 2011.

These complementation experiments were repeated using S. medicae inoculation, which has been reported to nodulate Medicago with higher efficiency. The results shown in FIG. 4B are complementation tests where the plants were inoculated with S. medicae. The S. medicae results confirm that the double mutant construct (L147D/L154D) complements poorly. Taken together, these results show that the hydrophobic patch in NFP is required for LCO recognition, and for functional symbiotic signaling.

### Example 4: Functional Characterization of CO and LCO Receptors Using Domain Swaps

The following example describes functional characterization of the Lotus LCO receptor NFR 1 and the Lotus CO receptor CERK6. This was done using domain swaps, and by measuring nodulation and defense (reactive oxygen species, ROS) responses to assess complementation.

### Materials and Methods

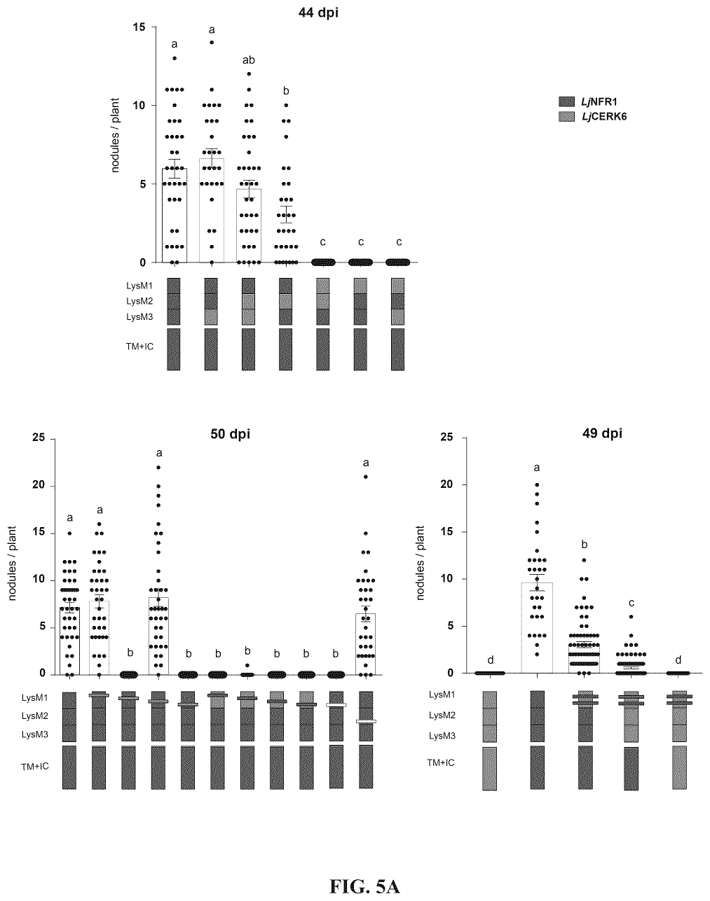

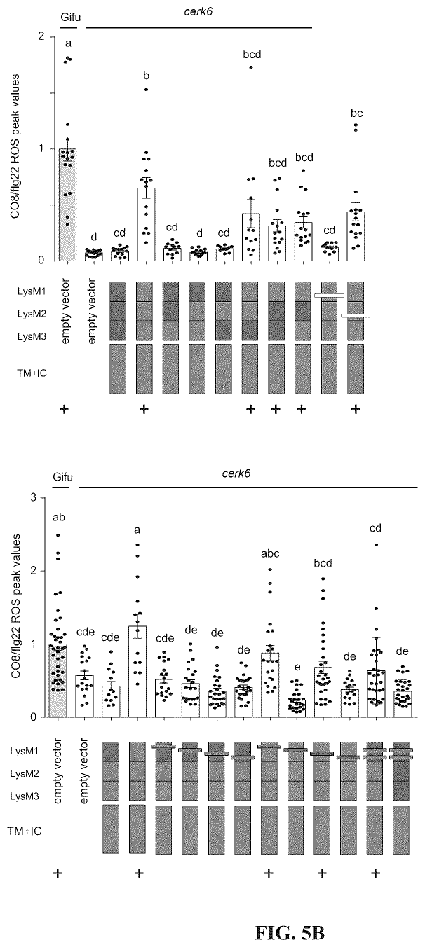

Complementation assay: Construct assembly, plant growth conditions, hairy root transformations, nodulation and ROS assays were generally conducted as described in Bozsoki et al. (2017) (Bozsoki Z, Cheng J, Feng F, Gysel K, Vinther M, Andersen K R, Oldroyd G, Blaise M, Radutoiu S, Stougaard J (2017) Receptor-mediated chitin perception in legume roots is functionally separable from Nod factor perception. Proc Natl Acad Sci 114: E8118-E8127). A general schematic of the construct is provided in FIG. 3, whereby the pNfr1 promoter was used for the constructs tested in nodulation assays, and the pCerk6 promoter was used for the constructs tested in ROS assays. For functional complementation of nfr1 and cerk6 mutants, only plants expressing the YFP marker protein from the transformation control (FIG. 3) were used. For the nodulation assays, nodules were counted on hairy root transformed L. japonicus nfr1-1 mutant roots after the indicated days post inoculation (dpi) (e.g., 44 dpi, 49 dpi, and 50 dpi) with M. loti R7A (FIG. 5A). For the ROS assays, transformed roots were harvested from individual plants, then the root material was divided into two halves, each half being tested for ROS response to CO8 or flg22. For each transformed plant the ratio of CO8 and flg22 elicited ROS peak values after were plotted normalized to the wild type sample, which was set as 1 (FIG. 5B). The tested chimeric receptors are depicted as shaded block diagrams in FIGS. 5A-5B.

### Results

FIGS. 5A-5B show results of functional studies measuring nodulation and defense using domain swaps between the Lotus LCO receptor NFR 1 and the Lotus CO receptor CERK6. FIG. 5A shows complementation experiments of a Lotus nfr1 single mutant with different domain-swapped protein constructs expressed under the pNFR1 promoter. Nodulation was used to assess complementation. FIG. 5B shows complementation experiments of a Lotus cerk6 single mutant with different domain-swapped protein constructs expressed under the pCerk6 promoter. The level of elicited ROS response was used to assess complementation. The results of these experiments show that the LysM1 domain of the NFR1 ectodomain is important to perceive both LCO (in the case of NFR1) and CO (in the case of CERK6) ligands.

Additional experiments, also depicted in FIGS. 5A-5B, swapped smaller sections, referred to as regions, of the domains. These experiments showed that two regions, namely region II and region IV, were particularly important for specific recognition of a ligand. Taken together, these results show that swapping either the entire LysM1 domain, or swapping only region II and region IV, is sufficient to convert a CO receptor into an LCO receptor.

### Example 5: Structural Characterization of Lotus CERK6 Ectodomain

The following example describes the structural characterization of the Lotus CERK6 protein ectodomain.

### Materials and Methods

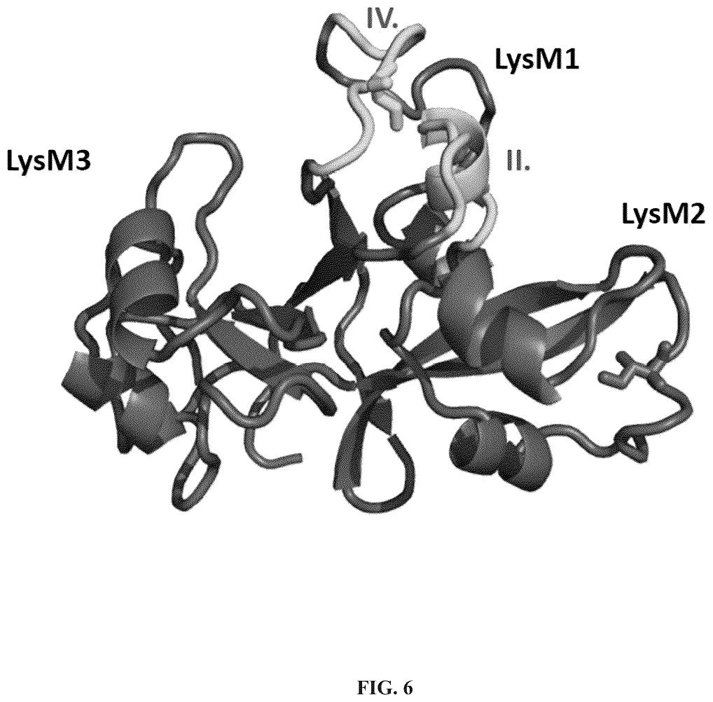

Modelling: The target LysM receptor amino acid sequence (Lotus CERK6) was aligned with a known receptor sequence (Medicago NFP). Then, the LysM1-3 domains of the target sequence were used as an input in SWISS-MODEL (Biasini 2014). The structural coordinate file (.pdb) of the Medicago NFP crystal structure as template file in SWISS-MODEL (Biasini 2014), and the modelling program was run using the command ‘Build Model’. The electrostatic surface potential of the output target (.pdb) model generated with SWISS-MODEL was calculated using PDB2PQR & APBS webservers (PMID: 21425296) and visualized in PyMol using APBS tools 2.1 (DeLano, W. L. 2002). The 3D structure of the Lotus CERK6 ectodomain is depicted in FIG. 6, and corresponds to that published by Bozsoki et al., 2017 (Bozsoki Z, Cheng J, Feng F, Gysel K, Vinther M, Andersen K R, Oldroyd G, Blaise M, Radutoiu S, Stougaard J (2017) Receptor-mediated chitin perception in legume roots is functionally separable from Nod factor perception. Proc Natl Acad Sci 114: E8118-E8127).

### Results

The 3D structure of CERK6 shows that region II and region IV are located adjacent to each other as shown in FIG. 6. This indicates the potential involvement of these two regions in a possible binding site.

### Example 6: Functional Characterization of LCO Receptors Using Domain Swaps

The following example describes functional characterization of the Lotus LCO receptor NFR1 and the Medicago LCO receptor LYK3. This was done using domain swaps, and by measuring nodulation to assess complementation.

### Materials and Methods

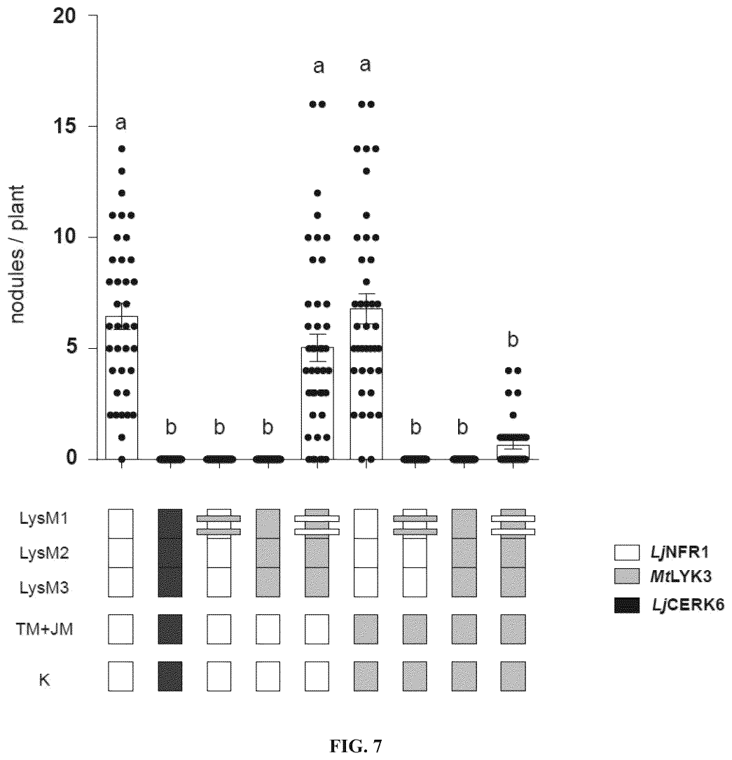

Complementation assay: Construct assembly, plant growth conditions, hairy root transformations, nodulation and ROS assays were generally conducted as described in Bozsoki et al. (2017) (Bozsoki Z, Cheng J, Feng F, Gysel K, Vinther M, Andersen K R, Oldroyd G, Blaise M, Radutoiu S, Stougaard J (2017) Receptor-mediated chitin perception in legume roots is functionally separable from Nod factor perception. Proc Natl Acad Sci 114: E8118-E8127). A general schematic of the construct is provided in FIG. 3, whereby the pNFR1 promoter was used to drive the chimeric constructs. The tested chimeric receptors in Lotus are depicted as block diagrams below the graph in FIG. 7, where NFR1 domains are shown in white and LYK3 domains are shown in grey, and transverse lines across the block depicting the LysM1 domain indicate sections II and IV. Unaltered Lotus CERK6 protein was used as a negative control (zero nodulation). Nodules were counted on hairy root transformed L. japonicus nfr1-1 mutant roots 35 days post inoculation with M. loti R7A. M. loti R7A is the cognate N-fixing bacterial strain for L. japonicus, and is not recognized by M. truncatula.

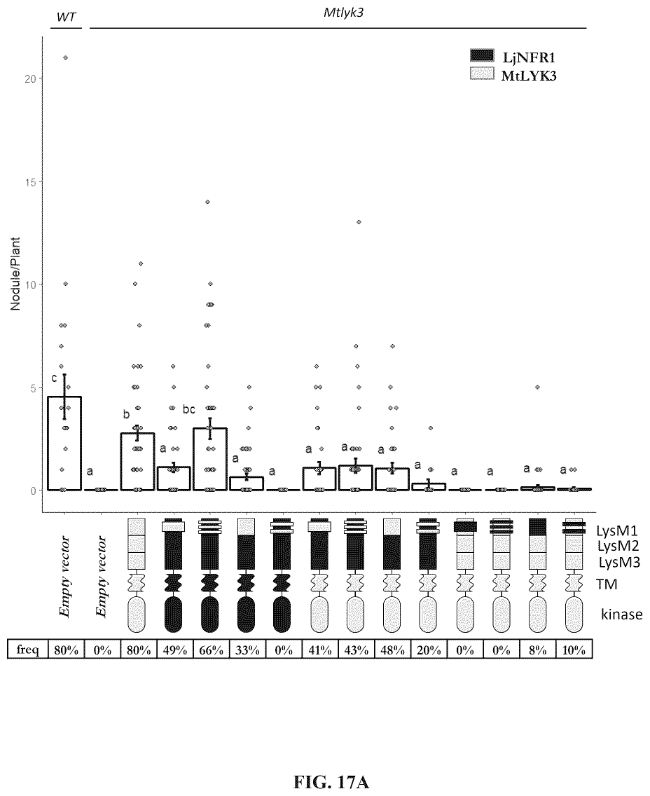

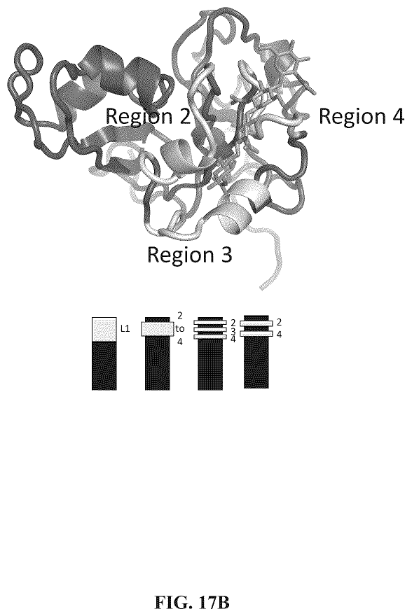

The tested chimeric receptors in Medicago are depicted as block diagrams in FIGS. 17A-17B, where NFR1 domains are shown in black, LYK3 domains are shown in grey, and transverse lines across the block depicting the LysM1 domain indicate regions II, III, and IV. The pLYK3 promoter was used to drive the chimeric constructs. Nodules were counted on hairy root transformed M. truncatula WT or M. truncatula lyk3 mutant roots 35 days post inoculation with S. meliloti. S. meliloti is the cognate N-fixing bacterial strain for M. truncatula, and is not recognized by L. japonicus.

### Results

FIG. 7 shows results of functional studies measuring nodulation using domain swaps between the Lotus LCO receptor NFR1 and the Medicago LCO receptor LYK3. In addition, LjCERK6 was included as a negative control (zero nodulation). In a Lotus nfr1-1 mutant, two recombinant receptors were able to complement. Domains of NFR1 (white) and LYK3 (grey) proteins were assembled in different chimeric constructs as shown in the diagram below the graph. Transversal lines across LysM1 show the where the section II and IV were derived from. CERK6 protein was used as control. Only chimeric receptors that contained regions II and IV of the LysM1 domain or the entire LysM1 domain from NFR1 complemented the nfr1 mutant when inoculated with M. loti. This indicates that the LysM1 domain is essential for allowing specific M. loti LCO recognition. The importance of region II and region IV is shown by a recombinant receptor entirely consisting of MtLYK3 except for LjNFR1 region II and region IV, which was able to functionally complement the nfr1 mutant, even though the efficiency did not reach wild-type levels. This chimeric MtLYK3 receptor shows that the swap of regions II and IV is sufficient to change the specificity to M. loti LCO. Taken together, these results indicate that the LysM1 domain is essential for recognizing those LCOs produced by the cognate N-fixing bacterial strain of a legume species. Moreover, regions II and IV are particularly important for this recognition, because when they are replaced, recognition is lost.

The results from FIG. 7 showed that a chimeric MtLYK3 receptor with a swap of regions II and IV (rightmost receptor on graph) was sufficient to change the specificity of an otherwise fully MtLYK3 protein, which would normally recognize S. meliloti LCO, to M. loti LCO. Engineering the LjNFR1 receptor with regions II and IV from MtLyk3 resulted in a receptor that was not able to recognize M. loti LCO (receptor third from left on graph and receptor third from right on graph). This chimeric receptor showed that the swap of regions II and IV was sufficient to abolish recognition of M. loti by an otherwise fully LjNFR1 protein.

FIG. 17A shows that in a M. truncatula lyk3 mutant, recombinant receptors containing sections of LysM1 that included region III from MtLYK3 were able to complement. Region III is six amino acids located between region II and region IV (FIG. 17B). Engineered receptors that had the entire LysM1 from MtLYK3 (e.g., the receptor sixth from left (empty vectors counted) or the receptor sixth from right), a section of LysM1 spanning region II to region IV from MtLYK3 (e.g., the receptor fourth from left (empty vectors counted) or the receptor eighth from right), or region II, region III, and region IV from MtLYK3 (e.g., the receptor fifth from left (empty vectors counted) or the receptor seventh from right) were all able to complement the symbiotic deficient phenotype of the M. truncatula lyk3 mutant when inoculated with S. meliloti. In contrast, engineered receptors containing only region II and region IV from MtLYK3 (e.g., the receptor seventh from left (empty vectors counted) or the receptor fifth from right) were not able to specifically recognize S. meliloti LCO. It was observed that the chimeric receptor with the MtLYK3 transmembrane and kinase domains (receptor fifth from right) had a very low complementation ability (3 plants of the 15 analyzed had 1 or 2 nodules), which was thought to be due to the high efficiency of these additional regions from MtLYK3. This result was interpreted as region III being required for specific and efficient recognition of S. meliloti LCO, but regions II and IV being critical. Engineering the MtLYK3 receptor with regions II to IV from LjNFR1 (the receptor fourth from right), regions II, III, and IV from LjNFR1 (the receptor fourth from right), the entire LysM1 from LjNFR1 (the receptor second from right), or regions II and IV from LjNFR1 (the rightmost receptor) resulted in a receptor that was not able to recognize S. meliloti LCO.

Overall, these results indicated that the LysM1 domain was essential for recognizing those LCOs produced by the cognate N-fixing bacterial strain of a legume species. When the chimeric receptors were expressed in M. truncatula, the regions II, III, and IV of the LysM1 domain were identified as particularly important for this recognition. Replacing regions II and IV were sufficient to obtain a loss of recognition. Replacing regions II, III, and IV were required to obtain gain of recognition for S. meliloti LCO and optimal functionality of the receptor.

### Example 7: Engineering Specific LCO Perception

The following example describes engineering of the Lotus receptor LYS11 (LjLYS11) to specifically perceive LCOs. This was done using domain swaps, by measuring ligand binding, and by measuring nodulation to assess complementation.

### Materials and Methods

LjLYS11 ectodomain production and purification: The LjLYS11 ectodomain (residues 26-234; SEQ ID NO:60) was codon-optimized for insect cell expression (Genscript, Piscataway, USA) and cloned into the pOET4 baculovirus transfer vector (Oxford Expression Technologies). The native LjLYS11 signal peptide was replaced with the gp64 signal peptide (SEQ ID NO:59) to facilitate secretion and a hexa-histidine (6×His; SEQ ID NO:61) tag was added to the C-terminus (LjLYS11-ecto (26-234), N-term gp64, C-term 6His=SEQ ID NO:56). Recombinant baculoviruses were produced in Sf9 cells (Spodoptera frugiperda) using the FlashBac Gold kit (Oxford Expression technologies) according to the manufacturer's instructions with Lipofectin (ThermoFisher Scientific) as a transfection reagent. Protein expression was performed as follows. Suspension-cultured Sf9 cells were maintained with shaking at 299 K in serum-free MAX-XP (BD-Biosciences, discontinued) or HyClone SFX (GE Healthcare) medium supplemented with 1% Pen-Strep (10000 U/ml, Life technologies) and 1% CD lipid concentrate (Gibco). Protein expression was induced by adding recombinant passage 3 virus once the Sf9 cells reached a cell density of 1.0*10\hat{ }6 cells/ml. After 5-7 days of expression, medium supernatant containing LjLYS11 ectodomains was harvested by centrifugation. This was followed by an overnight dialysis step against 50 mM Tris-HCl pH 8, 200 mM NaCl at 277 K. The LjLYS11 ectodomain was enriched by two subsequent steps of Ni-IMAC purification (HisTrap excel/HisTrap HP, both GE Healthcare). For crystallography experiments, N-glycans were removed using the endoglycosidase PNGase F (1:15 (w/w), room temperature, overnight). As a final purification step, LjLYS11 ectodomain was purified by SEC on a Superdex 200 10/300 or HiLoad Superdex 200 16/600 (both GE Healthcare) in phosphate buffered saline at pH 7.2 supplemented to a total of 500 mM NaCl (for binding assays) or 50 mM Tris-HCl, 200 mM NaCl (for crystallography).

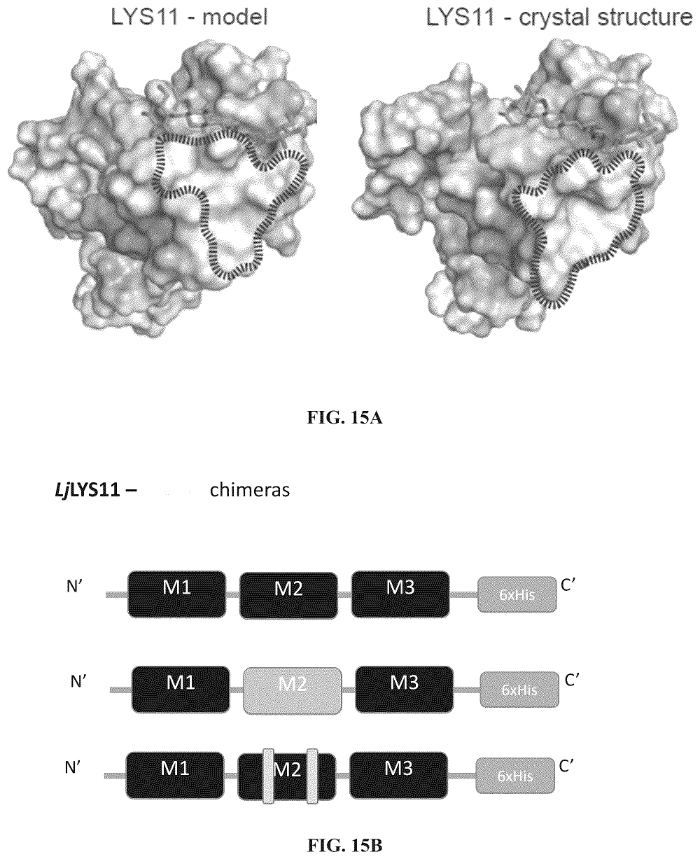

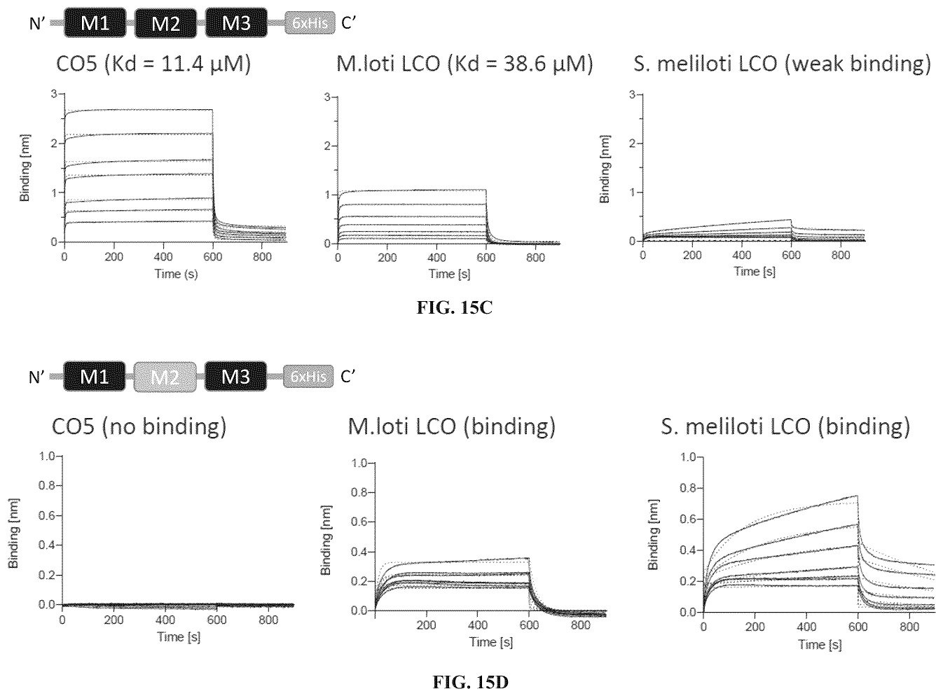

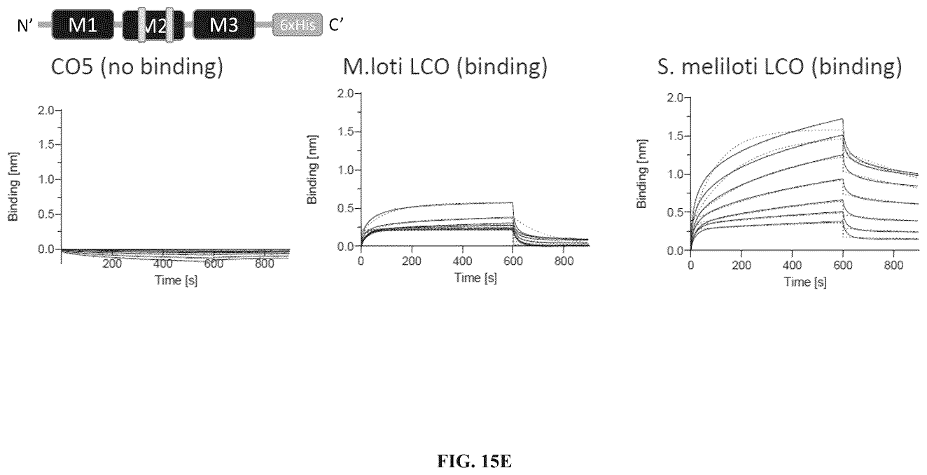

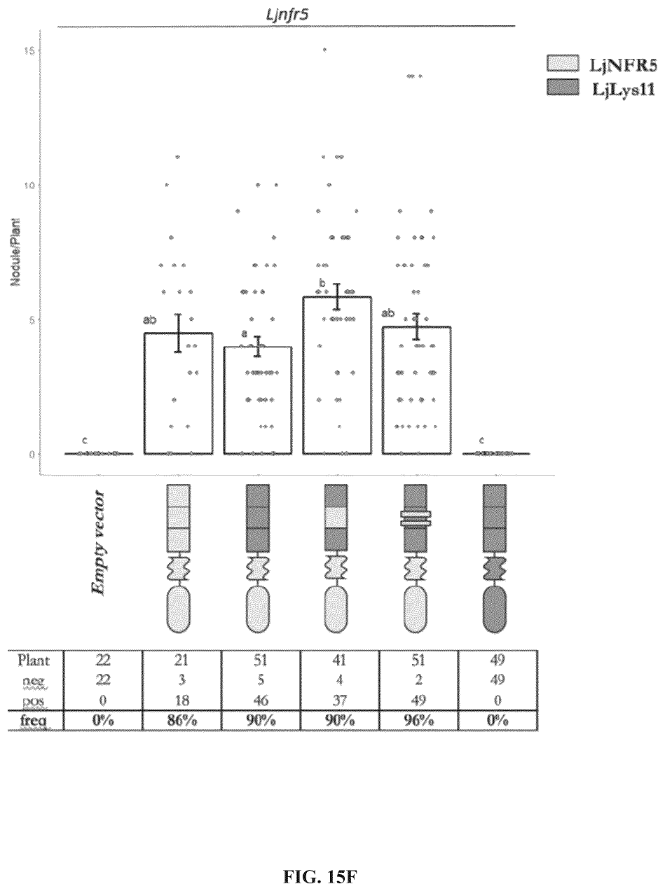

Biolayer interferometry (BLI): Binding of LjLYS11 ectodomain and domain-swapped versions of LjLYS11 ectodomain to ligands was measured on an Octet RED 96 system (Pall ForteBio). The ligands used were CO5 chitin oligomer (corresponding to the backbone of S. meliloti LCO-V), M. loti LCO, and S. meliloti LCO. S. meliloti LCO consists of a tetrameric/pentameric N-acetylglucosamine backbone that is O-sulfated on the reducing terminal residue, O-acetylated on the non-reducing terminal residue, and mono-N-acylated by unsaturated C16 acyl groups. M. loti LCO is a pentameric N-acetylglucosamine with a cis-vaccenic acid and a carbamoyl group at the non-reducing terminal residue together with a 2,4-O-acetylfucose at the reducing terminal residue. Biotinylated ligand conjugates were immobilized on streptavidin biosensors (kinetic quality, Pall ForteBio) at a concentration of 125-250 nM for 5 minutes. Data analysis was performed in GraphPad Prism 6 software (GraphPad Software, Inc.). Equilibrium dissociation constants derived from the steady-state were determined by applying a non-linear regression (one site, specific binding) to the response at equilibrium plotted against the protein concentration. Kinetic parameters were determined by non-linear regression (association followed by dissociation) on the subtracted data. The tested chimeric receptors are depicted as block diagrams in FIG. 15B, with LjLYS11 domains shown in black and LjNFR5 and above the binding assay results in FIGS. 15C-15E.

Complementation assay: The complementation assay was done as in Example 6. The tested chimeric receptors are depicted as block diagrams in FIG. 15F, where LjNFR5 domains are shown in light grey, LjLYS11 domains are shown in grey, and transverse lines across the block depicting the LysM2 domain indicate regions QLGDSYD (SEQ ID NO:63) and GV (SEQ ID NO: 64) from LjNFR5. Empty vector and full-length LjLYS11 were used as negative controls (zero nodulation). Nodules were counted on hairy root transformed L. japonicus nfr5-2 mutant roots 35 days post inoculation with M. loti R7A. M. loti R7A is the cognate N-fixing bacterial strain for L. japonicus.

### Results

Based on modelling and crystal structure determination of LjLYS11 ectodomain (FIG. 15A), it was predicted that the receptor would likely be a LCO receptor. To experimentally validate this prediction, binding experiments were performed. As shown in FIG. 15C, LjLYS11 ectodomain was able to bind CO5 (left graph), M. loti LCO (middle graph; M. loti is the cognate N-fixing bacterial strain for L. japonicus), and S. meliloti LCO (right graph; weak binding). This result indicated that the identified hydrophobic patch in the LjLYS11 ectodomain allowed it to bind LCO. Therefore, the hydrophobic patch was predictive of LCO-binding ability.

Next, it was tested whether stringent and specific LCO recognition could be engineered. For these tests, LjLYS11 ectodomains were engineered to contain parts of LjNFR5 receptors. Either the entire LysM2 or key residues from the LysM2 hydrophobic patch from LjLYS11 were replaced with the corresponding regions QLGDSYD (SEQ ID NO:63) and GV (SEQ ID NO:64) from LjNFR5, and ligand binding of these chimeric ectodomains was measured. As shown in FIG. 15D, replacing the entire LysM2 resulted in improved affinity to LCOs (both M. loti and S. meliloti LCOs), and resulted in a loss of ability to bind CO. A similar result was seen when only key residues of LysM2 were replaced (FIG. 15E).

Then, chimeric receptors were tested in planta. For these tests, the same chimeric LjLYS11 ectodomains were used (the entire LysM2, or key residues from LysM2 from LjLYS11 were replaced with the corresponding regions from LjNFR5) or the entire LjLYS11 ectodomain (LysM1, LysM2, and LysM3) was used, and these were attached to the transmembrane domain (wavy shape in schematic of FIG. 15F) and kinase domain (oval shape in schematic of FIG. 15F) of LjNFR5. In addition, full-length LjNFR5 and full-length LjLYS11 were tested. As shown in FIG. 15F, chimeric receptors with any one of these modifications (the receptors fourth from right, third from right, and second from right) retained their capacity to perceive the M. loti Nod factor and to initiate a symbiotic signaling event with similar efficiency as LjNFR5.

Interestingly, the chimeric LjLYS11/LjNFR5 ectodomains had different LCO binding kinetics with slow on/off rates that resembled the binding kinetics of M. truncatula NFP. As shown in FIG. 18D, slow on/off rate binding kinetics are thought to be important for functional symbiotic signaling. The fast on/off rate binding kinetics seen with hydrophobic patch mutants does not result in symbiotic signalling (FIG. 18E). Further, fast on/off kinetics also appear to be a hallmark of CO perception (FIG. 18C). As shown in FIG. 18F, NFP shared the cysteine bridge connectivity pattern and the overall arrangement of the scaffold with other LysM receptor kinases involved in chitin-elicited defence signalling. This result supported the hypothesis that despite their different function, these LysM receptors shared a common evolutionary origin (Zhang, X.-C. et al. Molecular evolution of lysin motif-type receptor-like kinases in plants. Plant Physiol. 144, 623-636 (2007)). The shared structural features of the LysM receptors provided further support for the ability to engineer these receptors to have different binding kinetics. For example, the altered binding kinetics observed with the chimeric LjLYS11/LjNFR5 ectodomains indicate that receptors can be engineered to have LCO binding kinetics characteristics of functional symbiotic signalling.

Taken together, the results seen with chimeric LjLYS11/LjNFR5 ectodomains show that LysM2 engineering can create receptors with higher stringency toward LCO as well as higher specificity toward LCO.

### Example 8: Identifying Target LysM Receptors for Engineering

The following example describes homology modelling in barley (H. vulgare) to identify target LysM receptors for use in engineering.

### Materials and Methods

Modelling: Homology modelling was performed with SWISS-MODEL (Biasini, M. et al. SWISS-MODEL: modelling protein tertiary and quaternary structure using evolutionary information. Nucleic Acids Res. 42, W252-W258 (2014)). For barley RLK10 (HvRLK10), the crystal structure of Medicago NFP served as the template model onto which the amino acid sequence of the target receptor was mapped. For barley RLK4 (HvRLK4), the crystal structure of Medicago LYK3 served as the template model onto which the amino acid sequence of the target receptor was mapped. The output pdb filled structural model was generated and its electrostatic surface potential was calculated using the PDB2PQR and APBS webservers (PMID: 21425296). The results were visualized in PyMol using APBS tools 2.1 (DeLano, W. L. (2002). PyMOL. DeLano Scientific, San Carlos, CA, 700.).

Expression and purification of ectodomain: The HvRLK10 ectodomain (residues 25-231; SEQ ID NO:66) was codon-optimized for insect cell expression (Genscript, Piscataway, USA) and cloned into the POET4 baculovirus transfer vector (Oxford Expression Technologies). The native HvRLK10 signal peptide was replaced with the gp64 signal peptide (SEQ ID NO:59) to facilitate secretion and a hexa-histidine (6×HIS; SEQ ID NO:61) tag was added to the C-terminus (HvRLK10-ecto (25-231), N-term gp64, C-term 6His=SEQ ID NO:65). The HvRLK4 ectodomain (residues 27-228; SEQ ID NO:68) was codon-optimized for insect cell expression (Genscript, Piscataway, USA) and cloned into the POET4 baculovirus transfer vector (Oxford Expression Technologies). The native HvRLK4 signal peptide was replaced with the gp64 signal peptide (SEQ ID NO:59) to facilitate secretion and a hexa-histidine (6×HIS; SEQ ID NO:61) tag was added to the C-terminus (RLK4-ecto (27-228), N-term gp64, C-term 6His=SEQ ID NO: 67). Recombinant baculoviruses were produced in Sf9 cells (Spodoptera frugiperda) using the FlashBac Gold kit (Oxford Expression technologies) according to the manufacturer's instructions with Lipofectin (ThermoFisher Scientific) as a transfection reagent.

Protein expression was performed as follows. Suspension-cultured Sf9 cells were maintained with shaking at 299 K in serum-free MAX-XP (BD-Biosciences, discontinued) or HyClone SFX (GE Healthcare) medium supplemented with 1% Pen-Strep (10000 U/ml, Life technologies) and 1% CD lipid concentrate (Gibco). Protein expression was induced by adding recombinant passage 3 virus once the Sf9 cells reached a cell density of 1.0*10\hat{ }6 cells/ml. After 5-7 days of expression, medium supernatant containing HvRLK10 ectodomain or HvRLK4 ectodomain was harvested by centrifugation. This was followed by an overnight dialysis step against 50 mM Tris-HCl pH 8, 200 mM NaCl at 277 K. The HvRLK10 ectodomain or HvRLK4 ectodomain was enriched by two subsequent steps of Ni-IMAC purification (HisTrap excel/HisTrap HP, both GE Healthcare).

Biolayer interferometry (BLI): Binding of HvRLK10 ectodomain or HvRLK4 ectodomain to ligands was measured on an Octet RED 96 system (Pall ForteBio). The ligands used were CO5 chitin oligomer (corresponding to the backbone of S. meliloti LCO-V), M. loti LCO, and S. meliloti LCO. S. meliloti LCO consists of a tetrameric/pentameric N-acetylglucosamine backbone that is O-sulfated on the reducing terminal residue, O-acetylated on the non-reducing terminal residue, and mono-N-acylated by unsaturated C16 acyl groups. M. loti LCO is a pentameric N-acetylglucosamine with a cis-vaccenic acid and a carbamoyl group at the non-reducing terminal residue together with a 2,4-O-acetylfucose at the reducing terminal residue. Biotinylated ligand conjugates were immobilized on streptavidin biosensors (kinetic quality, Pall ForteBio) at a concentration of 125-250 nM for 5 minutes. Data analysis was performed in GraphPad Prism 6 software (GraphPad Software, Inc.). Equilibrium dissociation constants derived from the steady-state were determined by applying a non-linear regression (one site, specific binding) to the response at equilibrium plotted against the protein concentration. Kinetic parameters were determined by non-linear regression (association followed by dissociation) on the subtracted data.

### Results

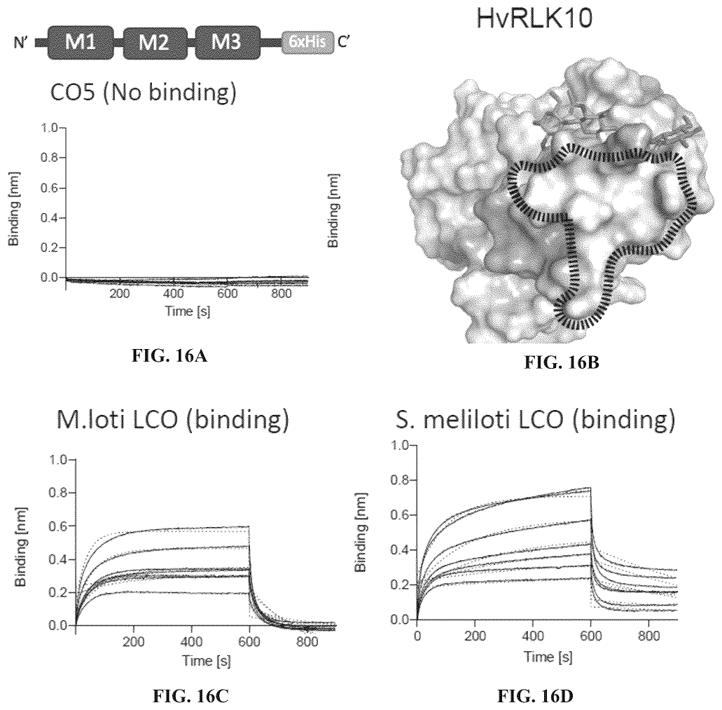

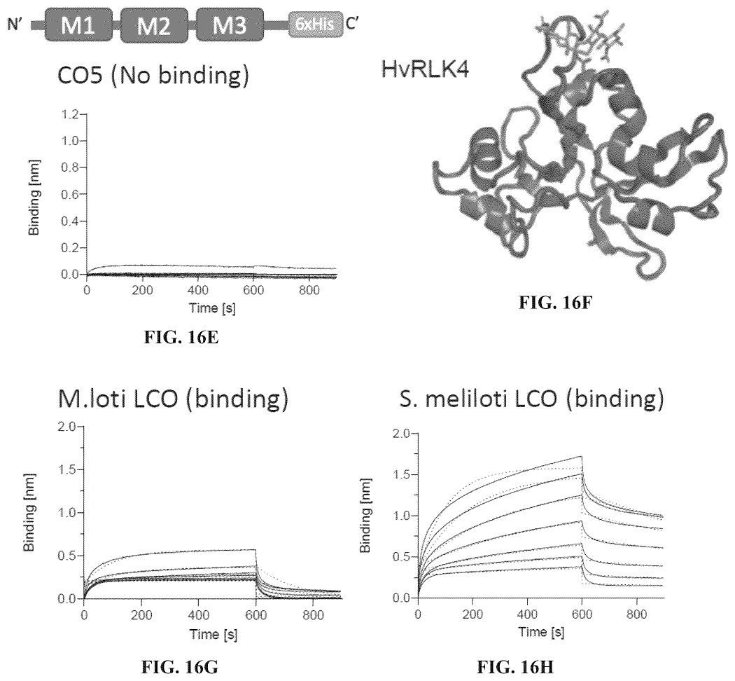

Homology modelling of all ten barley LysM receptor-like kinases (RLKs) was done using the Medicago NFP structure as a template. Of the barley LysM RLKs, HvRLK10 was the receptor that was closest to Medicago NFP and modelled the best using this approach. FIG. 16B shows homology modelling results for HvRLK10, which revealed that the hydrophobic patch was indeed present in the equivalent positions immediately below the LysM2 domain of this receptor. This clear hydrophobic patch indicated that HvRLK10 was a NFP/NFR5 type of LCO receptor.

To experimentally validate this prediction, the HvRLK10 ectodomain was expressed and purified for use in binding experiments (ectodomain schematic shown at top of FIG. 16A). The HvRLK10 ectodomain was shown to bind both M. loti LCO (FIG. 16C) and S. meliloti LCO (FIG. 16D). In contrast, the HvRLK10 ectodomain did not bind CO5 (FIG. 16A). These results provided functional characterization of the HvRLK10 ectodomain, and showed that it bound LCOs, but not COs. The results confirmed that the HvRLK10 receptor was a LCO receptor, as had been predicted by the homology modelling.

In addition, homology modelling of all ten barley LysM RLKs was done using the Medicago LYK3 structure as a template. HvRLK4 was the receptor that was closest to Medicago LYK3 and modelled the best using this approach. FIG. 16F shows homology modelling results for HvRLK4. These results indicated that this receptor was a NFR1/LYK3 type of LCO receptor.

To experimentally validate this prediction, the HvRLK4 ectodomain was expressed and purified for use in binding experiments (ectodomain schematic shown at top of FIG. 16E). The HvRLK4 ectodomain was shown to bind both M. loti LCO (FIG. 16G) and S. meliloti LCO (FIG. 16H). In contrast, the HvRLK4 ectodomain did not bind CO5 (FIG. 16E). These results provided functional characterization of the HvRLK4 ectodomain, and showed that it bound LCOs, but not COs. The results confirmed that the HvRLK4 receptor was a LCO receptor, as had been predicted by the homology modelling.

Both HvRLK10 and HvRLK4 were initially identified by homology modelling, and then confirmed to be LCO receptors by biochemical characterization. HvRLK10 and HvRLK4 therefore represent promising target receptors for engineering in barley particularly for engineering receptors that recognize LCOs in a manner similar to the donor receptors used to select them, Medicago NFP and Medicago LYK3, respectively.

Overall, these results show that the homology modelling approach can be used to identify LCO receptors of both the NFP/NFR5 type and the NFR1/LYK3 type specifically and that this method may be used to identify good target LysM receptor to modify to alter a desired receptor characteristic to be that of the donor LysM receptor used to select the target LysM receptor.

### Example 9: Exemplary Structural Alignment to Identify of Target Residues to Modify for Insertion of a Hydrophobic Patch

One of skill in the art would have no difficulty applying the teachings of this disclosure to genetically alter LysM receptors to include a hydrophobic patch or alter an existing hydrophobic patch. Exemplary steps would be:

1. Align the target LysM receptor amino acid sequence with one or more known LCO receptor sequences (See, e.g., FIGS. 8A-8C, FIGS. 9A-9B, FIGS. 10A-10B, FIGS. 11A-11B) to identify the sequence of the LysM1-3 domains in the target amino acid sequence.

Applying this step to the HvLysM-RLK2/37-247 sequence produced the following amino acid sequence:

>HvLysM-RLK2/37-247
(SEQ ID NO: 98)
SVEGFNCSANGTYPCQAYALYRAGLAGVPPDLSAAGDLFGVSRFMLAHA
NNLSTSAAPAAGQPLLVPLQCGCPSGSPNAYAPTQYQISSGDTFWIVSV
TKLQNLTQYQAVERVNPTVVPTKLEVGDMVTFPIFCQCPTAAQNATALV
TYVMQQGDTYASIAAAFAVDAQSLVSLNGPEQGTQLFSEILVPLRRQVP
KWLPPIVTRNDASAT

2. Use the LysM1-3 domain amino acid sequence as the input sequence to be modeled in an appropriate molecular modeling program such as SWISS-MODEL (Biasini 2014). SWISS-MODEL can be readily accessed at swissmodel.expasy.org under interactive #structure.
3. Input the structural template to the molecular modelling program, for example from a structural coordinate file (e.g., a pdb format file).

The HvLysM-RLK2/37-247 LysM1-3 domain amino acid sequence was entered into SWISS-MODEL as was the Medicago NFP receptor ectodomain crystal structure.pdb file (the atomic coordinates are reproduced at the end of the specification). The SWISS-MODEL program was run by the command ‘Build Model’. The Medicago NFP receptor ectodomain crystal structure was chosen as it has a known hydrophobic patch. One of skill in the art can readily select others based upon the teachings in this specification.

4. Optionally create an electrostatic surface potential of the target model and structurally align with a structure with chitin (or glycan) bound to the LysM2 domain to align the ligand binding grooves.

An electrostatic surface potential of the output target (.pdb) model generated with SWISS-MODEL was calculated using PDB2PQR & APBS webservers (PMID: 21425296) and visualized in PyMol using APBS tools 2.1 (DeLano, W. L. 2002). The AtCERK1 ectodomain structure (PDB coordinates 4EBZ) which has the chitin bound in the structure was aligned to the target model in PyMol. One of skill in the art would readily understand the position of the chitin binding domain as the LysM chitin binding motif is defined structurally in Liu et al. Science 2012 for AtCERK1. This aligned the chitin (CO4) ligand in the LysM2 ligand binding groove of the target model. FIGS. 13A-13B show the PyMol visualization of the LysM1-3 domains of the HvLysM-RLK2/37-247 model with the LysM1, LysM2, and LysM3 domains labeled (FIG. 13A), and the electrostatic surface potential of the model with chitin modeled in the binding groove (FIG. 13B).

5. Select the residues from the alignment in the target model that align with the known hydrophobic patch.

From the sequence alignment (1), structural alignment of the target model with the crystal structure of Medicago NFP and the electrostatic surface potential information (5) the hydrophobic patch was identified (with the placed chitin from AtCERK1 as reference for locating the CO binding groove as shown in (FIG. 13B). Hot-spot residues corresponding to the Medicago NFP ectodomain hydrophobic patch (L147, L151, L152, L154, T156, K157 and V158) were identified based on the amino acid being within 3 Å of an alpha carbon of a known hydrophobic patch amino acid residue (Medicago NFP L147, L151, L152, L154, T156, K157 and V158) in the structural alignment. As one of skill in the art would appreciate, residues like lysine (K) and arginine (R) that are not classically characterized as hydrophobic, do contain hydrophobic properties related to the Calpha, Cbeta, Cgamma, Cdelta and Cepsilon atoms that might be important for LCO binding, selectivity, promiscuity, stringency, and affinity and therefore are still potentially important (e.g., K157 of the Medicago NFP hydrophobic patch). The identified residues in the HvLysM-RLK2/37-247 model (bolded in FIG. 13C) can be mutated, preferably with additional modeling, to obtain engineered LCO binding, LCO/CO selectivity, LCO promiscuity, LCO stringency, LCO affinity. One of skill in the art would appreciate that similar structural modeling can be used to structurally align LysM1 domains to identify regions II and IV in order to substitute and alter specificity, affinity and selectivity of a target LysM receptor for an agonist.

The Medicago NFP ectodomain crystal structure

| | | | | | | | | | | | | | | |
|---|---|---|---|---|---|---|---|---|---|---|---|---|---|---|
| LINK | | C1 | NAG A | 2076 | | O4 | NAG A | 1076 | | | | | | |
| LINK | | C1 | NAG A | 2123 | | O4 | NAG A | 1123 | | | | | | |
| LINK | | C1 | NAG A | 2144 | | O4 | NAG A | 1144 | | | | | | |
| LINK | | C1 | NAG A | 2228 | | O4 | NAG A | 1228 | | | | | | |
| LINK | | C1 | NAG A | 1076 | | ND2 | ASN A | 76 | | | | | | |
| LINK | | C1 | NAG A | 1123 | | ND2 | ASN A | 123 | | | | | | |
| LINK | | C1 | NAG A | 1144 | | ND2 | ASN A | 144 | | | | | | |
| LINK | | C1 | NAG A | 1228 | | ND2 | ASN A | 228 | | | | | | |
| SSBOND | 1 | CYS A | 50 | CYS A | 115 | | | | | | | | | |
| SSBOND | 2 | CYS A | 58 | CYS A | 177 | | | | | | | | | |
| SSBOND | 3 | CYS A | 113 | CYS A | 175 | | | | | | | | | |
| CRYST1 | 77.410 | 98.160 | 71.890 | 90.00 | 90.00 | 90.00 | C | 2 | 2 | 21 | | | | |
| SCALE1 | 0.012918 | 0.000000 | 0.000000 | 0.00000 | | | | | | | | | | |
| SCALE2 | 0.000000 | 0.010187 | 0.000000 | 0.00000 | | | | | | | | | | |
| SCALE3 | 0.000000 | 0.000000 | 0.013910 | 0.00000 | | | | | | | | | | |
| ATOM | 1 | N | SER A | 44 | 47.490 | 34.635 | 22.387 | | | 1.00 | 132.78 | | A | N |
| ANISOU | 1 | N | SER A | 44 | 14506 | 18967 | 16976 | −687 | −2436 | | | 7410 | A | N |
| ATOM | 2 | CA | SER A | 44 | 48.575 | 35.497 | 22.832 | | | 1.00 | 128.50 | | A | C |
| ANISOU | 2 | CA | SER A | 44 | 14079 | 17864 | 16882 | −714 | −2577 | | | 7389 | A | C |
| ATOM | 3 | C | SER A | 44 | 49.218 | 34.959 | 24.094 | | | 1.00 | 121.82 | | A | C |
| ANISOU | 3 | C | SER A | 44 | 13323 | 16878 | 16087 | −720 | −2520 | | | 7111 | A | C |
| ATOM | 4 | O | SER A | 44 | 50.272 | 34.341 | 24.053 | | | 1.00 | 114.50 | | A | O |
| ANISOU | 4 | O | SER A | 44 | 12311 | 16241 | 14951 | −801 | −2396 | | | 7114 | A | O |
| ATOM | 5 | CB | SER A | 44 | 48.060 | 36.911 | 23.094 | | | 1.00 | 126.83 | | A | C |
| ANISOU | 5 | CB | SER A | 44 | 13992 | 17346 | 16852 | −547 | −2697 | | | 7268 | A | C |
| ATOM | 6 | OG | SER A | 44 | 49.010 | 37.665 | 23.828 | | | 1.00 | 128.24 | | A | O |
| ANISOU | 6 | OG | SER A | 44 | 14237 | 17006 | 17480 | −596 | −2848 | | | 7359 | A | O |
| ATOM | 7 | N | GLU A | 45 | 48.551 | 35.184 | 25.216 | | | 1.00 | 120.97 | | A | N |
| ANISOU | 7 | N | GLU A | 45 | 13377 | 16340 | 16246 | −626 | −2607 | | | 6862 | A | N |
| ATOM | 8 | CA | GLU A | 45 | 49.040 | 34.751 | 26.516 | | | 1.00 | 122.62 | | A | C |
| ANISOU | 8 | CA | GLU A | 45 | 13672 | 16343 | 16575 | −651 | −2584 | | | 6624 | A | C |
| ATOM | 9 | C | GLU A | 45 | 49.060 | 33.238 | 26.635 | | | 1.00 | 120.68 | | A | C |
| ANISOU | 9 | C | GLU A | 45 | 13458 | 16423 | 15972 | −535 | −2435 | | | 6316 | A | C |
| ATOM | 10 | O | GLU A | 45 | 48.274 | 32.543 | 25.998 | | | 1.00 | 122.15 | | A | O |
| ANISOU | 10 | O | GLU A | 45 | 13634 | 16922 | 15853 | −411 | −2368 | | | 6224 | A | O |
| ATOM | 11 | CB | GLU A | 45 | 48.163 | 35.326 | 27.628 | | | 1.00 | 121.51 | | A | C |
| ANISOU | 11 | CB | GLU A | 45 | 13688 | 15639 | 16840 | −586 | −2726 | | | 6442 | A | C |
| ATOM | 12 | CG | GLU A | 45 | 48.212 | 36.836 | 27.755 | | | 1.00 | 127.27 | | A | C |
| ANISOU | 12 | CG | GLU A | 45 | 14403 | 15998 | 17957 | −715 | −2869 | | | 6708 | A | C |
| ATOM | 13 | CD | GLU A | 45 | 49.555 | 37.327 | 28.243 | | | 1.00 | 132.32 | | A | C |
| ANISOU | 13 | CD | GLU A | 45 | 14930 | 16696 | 18650 | −952 | −2852 | | | 6959 | A | C |
| ATOM | 14 | OE1 | GLU A | 45 | 49.586 | 38.123 | 29.205 | | | 1.00 | 136.87 | | A | O |
| ANISOU | 14 | OE1 | GLU A | 45 | 15373 | 17434 | 19196 | −1069 | −2872 | | | 7310 | A | O |
| ATOM | 15 | OE2 | GLU A | 45 | 50.579 | 36.907 | 27.669 | | | 1.00 | 131.61 | | A | O1− |
| ANISOU | 15 | OE2 | GLU A | 45 | 14877 | 16507 | 18623 | −1022 | −2821 | | | 6805 | A | O1− |
| ATOM | 16 | N | THR A | 46 | 49.991 | 32.743 | 27.440 | | | 1.00 | 124.90 | | A | N |
| ANISOU | 16 | N | THR A | 46 | 14032 | 16873 | 16550 | −581 | −2389 | | | 6151 | A | N |
| ATOM | 17 | CA | THR A | 46 | 50.130 | 31.318 | 27.669 | | | 1.00 | 110.09 | | A | C |
| ANISOU | 17 | CA | THR A | 46 | 12173 | 15318 | 14337 | −492 | −2238 | | | 5885 | A | C |
| ATOM | 18 | C | THR A | 46 | 49.107 | 30.887 | 28.697 | | | 1.00 | 103.70 | | A | C |
| ANISOU | 18 | C | THR A | 46 | 11505 | 14437 | 13459 | −283 | −2255 | | | 5524 | A | C |
| ATOM | 19 | O | THR A | 46 | 49.388 | 30.892 | 29.895 | | | 1.00 | 100.49 | | A | O |
| ANISOU | 19 | O | THR A | 46 | 11098 | 14391 | 12694 | −175 | −2139 | | | 5343 | A | O |
| ATOM | 20 | CB | THR A | 46 | 51.526 | 30.968 | 28.209 | | | 1.00 | 90.86 | | A | C |
| ANISOU | 20 | CB | THR A | 46 | 9735 | 12798 | 11989 | −599 | −2195 | | | 5824 | A | C |
| ATOM | 21 | OG1 | THR A | 46 | 51.550 | 29.598 | 28.624 | | | 1.00 | 82.38 | | A | O |
| ANISOU | 21 | OG1 | THR A | 46 | 8675 | 12043 | 10583 | −499 | −2045 | | | 5566 | A | O |
| ATOM | 22 | CG2 | THR A | 46 | 51.868 | 31.850 | 29.398 | | | 1.00 | 77.97 | | A | C |
| ANISOU | 22 | CG2 | THR A | 46 | 8237 | 10610 | 10779 | −617 | −2337 | | | 5681 | A | C |
| ATOM | 23 | N | ASN A | 47 | 47.911 | 30.561 | 28.230 | | | 1.00 | 89.04 | | A | N |
| ANISOU | 23 | N | ASN A | 47 | 9765 | 12123 | 11942 | −226 | −2396 | | | 5399 | A | N |
| ATOM | 24 | CA | ASN A | 47 | 46.844 | 30.100 | 29.104 | | | 1.00 | 83.06 | | A | C |
| ANISOU | 24 | CA | ASN A | 47 | 9114 | 11294 | 11149 | −31 | −2436 | | | 5105 | A | C |
| ATOM | 25 | C | ASN A | 47 | 46.404 | 31.034 | 30.221 | | | 1.00 | 80.85 | | A | C |
| ANISOU | 25 | C | ASN A | 47 | 8941 | 10474 | 11302 | 9 | −2591 | | | 5004 | A | C |
| ATOM | 26 | O | ASN A | 47 | 46.004 | 32.161 | 29.974 | | | 1.00 | 86.84 | | A | O |
| ANISOU | 26 | O | ASN A | 47 | 9724 | 11126 | 12145 | 112 | −2671 | | | 5008 | A | O |
| ATOM | 27 | CB | ASN A | 47 | 47.083 | 28.674 | 29.595 | | | 1.00 | 84.42 | | A | C |
| ANISOU | 27 | CB | ASN A | 47 | 9345 | 11671 | 11058 | 72 | −2332 | | | 4752 | A | C |
| ATOM | 28 | CG | ASN A | 47 | 46.851 | 27.659 | 28.508 | | | 1.00 | 86.04 | | A | C |
| ANISOU | 28 | CG | ASN A | 47 | 9512 | 12360 | 10818 | 171 | −2218 | | | 4673 | A | C |
| ATOM | 29 | OD1 | ASN A | 47 | 46.815 | 26.461 | 28.751 | | | 1.00 | 87.76 | | A | O |
| ANISOU | 29 | OD1 | ASN A | 47 | 9702 | 12671 | 10972 | 219 | −2256 | | | 4778 | A | O |
| ATOM | 30 | ND2 | ASN A | 47 | 46.678 | 28.143 | 27.293 | | | 1.00 | 88.00 | | A | N |
| ANISOU | 30 | ND2 | ASN A | 47 | 9761 | 12911 | 10762 | 198 | −2077 | | | 4483 | A | N |
| ATOM | 31 | N | PHE A | 48 | 46.498 | 30.555 | 31.453 | | | 1.00 | 77.89 | | A | N |
| ANISOU | 31 | N | PHE A | 48 | 8628 | 9762 | 11206 | −71 | −2633 | | | 4906 | A | N |
| ATOM | 32 | CA | PHE A | 48 | 45.991 | 31.292 | 32.602 | | | 1.00 | 75.80 | | A | C |
| ANISOU | 32 | CA | PHE A | 48 | 8491 | 9027 | 11281 | 11 | −2743 | | | 4640 | A | C |
| ATOM | 33 | C | PHE A | 48 | 46.493 | 32.702 | 32.781 | | | 1.00 | 86.98 | | A | C |
| ANISOU | 33 | C | PHE A | 48 | 9926 | 10018 | 13104 | −64 | −2869 | | | 4809 | A | C |
| ATOM | 34 | O | PHE A | 48 | 47.684 | 32.980 | 32.841 | | | 1.00 | 94.16 | | A | O |
| ANISOU | 34 | O | PHE A | 48 | 10808 | 10921 | 14046 | −4 | −2923 | | | 4965 | A | O |
| ATOM | 35 | CB | PHE A | 48 | 46.320 | 30.503 | 33.864 | | | 1.00 | 65.86 | | A | C |
| ANISOU | 35 | CB | PHE A | 48 | 7303 | 7645 | 10076 | −14 | −2713 | | | 4362 | A | C |
| ATOM | 36 | CG | PHE A | 48 | 46.172 | 29.034 | 33.683 | | | 1.00 | 63.01 | | A | C |
| ANISOU | 36 | CG | PHE A | 48 | 6933 | 7684 | 9323 | 87 | −2597 | | | 4158 | A | C |
| ATOM | 37 | CD2 | PHE A | 48 | 47.112 | 28.323 | 32.976 | | | 1.00 | 62.96 | | A | C |
| ANISOU | 37 | CD2 | PHE A | 48 | 6835 | 8073 | 9013 | 6 | −2469 | | | 4295 | A | C |
| ATOM | 38 | CD1 | PHE A | 48 | 45.066 | 28.381 | 34.164 | | | 1.00 | 60.68 | | A | C |
| ANISOU | 38 | CD1 | PHE A | 48 | 6713 | 7401 | 8941 | 267 | −2609 | | | 3827 | A | C |
| ATOM | 39 | CE2 | PHE A | 48 | 46.969 | 26.974 | 32.779 | | | 1.00 | 60.62 | | A | C |
| ANISOU | 39 | CE2 | PHE A | 48 | 6536 | 8154 | 8341 | 108 | −2350 | | | 4087 | A | C |
| ATOM | 40 | CE1 | PHE A | 48 | 44.914 | 27.031 | 33.971 | | | 1.00 | 58.32 | | A | C |
| ANISOU | 40 | CE1 | PHE A | 48 | 6413 | 7479 | 8266 | 348 | −2488 | | | 3607 | A | C |
| ATOM | 41 | CZ | PHE A | 48 | 45.864 | 26.329 | 33.274 | | | 1.00 | 58.33 | | A | C |
| ANISOU | 41 | CZ | PHE A | 48 | 6338 | 7852 | 7972 | 270 | −2349 | | | 3723 | A | C |
| ATOM | 42 | N | THR A | 49 | 45.510 | 33.574 | 32.909 | | | 1.00 | 82.31 | | A | N |
| ANISOU | 42 | N | THR A | 49 | 9387 | 9064 | 12823 | −188 | −2920 | | | 4756 | A | N |
| ATOM | 43 | CA | THR A | 49 | 45.676 | 34.992 | 33.123 | | | 1.00 | 98.91 | | A | C |
| ANISOU | 43 | CA | THR A | 49 | 11496 | 10792 | 15293 | −304 | −3029 | | | 4935 | A | C |
| ATOM | 44 | C | THR A | 49 | 44.436 | 35.410 | 33.889 | | | 1.00 | 99.11 | | A | C |
| ANISOU | 44 | C | THR A | 49 | 11606 | 10480 | 15570 | −157 | −3127 | | | 4794 | A | C |
| ATOM | 45 | O | THR A | 49 | 43.357 | 34.846 | 33.711 | | | 1.00 | 96.68 | | A | O |
| ANISOU | 45 | O | THR A | 49 | 11283 | 9961 | 15491 | −209 | −3212 | | | 5006 | A | O |
| ATOM | 46 | CB | THR A | 49 | 45.709 | 35.771 | 31.797 | | | 1.00 | 109.76 | | A | C |
| ANISOU | 46 | CB | THR A | 49 | 12731 | 12391 | 16580 | −449 | −3025 | | | 5389 | A | C |
| ATOM | 47 | OG1 | THR A | 49 | 44.666 | 35.294 | 30.938 | | | 1.00 | 106.67 | | A | O |
| ANISOU | 47 | OG1 | THR A | 49 | 12281 | 12270 | 15978 | −332 | −3012 | | | 5538 | A | O |
| ATOM | 48 | CG2 | THR A | 49 | 47.041 | 35.602 | 31.095 | | | 1.00 | 111.16 | | A | C |
| ANISOU | 48 | CG2 | THR A | 49 | 12807 | 12921 | 16508 | −598 | −2915 | | | 5529 | A | C |
| ATOM | 49 | N | CYS A | 50 | 44.549 | 36.431 | 34.711 | | | 1.00 | 92.80 | | A | N |
| ANISOU | 49 | N | CYS A | 50 | 10892 | 9630 | 14739 | 27 | −3116 | | | 4435 | A | N |
| ATOM | 50 | CA | CYS A | 50 | 43.388 | 36.843 | 35.468 | | | 1.00 | 92.80 | | A | C |
| ANISOU | 50 | CA | CYS A | 50 | 10978 | 9292 | 14991 | 171 | −3192 | | | 4217 | A | C |
| ATOM | 51 | C | CYS A | 50 | 42.305 | 37.431 | 34.561 | | | 1.00 | 97.62 | | A | C |
| ANISOU | 51 | C | CYS A | 50 | 11539 | 9963 | 15591 | 278 | −3243 | | | 4441 | A | C |
| ATOM | 52 | O | CYS A | 50 | 42.612 | 38.040 | 33.542 | | | 1.00 | 93.35 | | A | O |
| ANISOU | 52 | O | CYS A | 50 | 10906 | 9567 | 14996 | 182 | −3258 | | | 4821 | A | O |
| ATOM | 53 | CB | CYS A | 50 | 43.807 | 37.863 | 36.515 | | | 1.00 | 100.59 | | A | C |
| ANISOU | 53 | CB | CYS A | 50 | 12040 | 9808 | 16371 | 64 | −3262 | | | 4119 | A | C |
| ATOM | 54 | SG | CYS A | 50 | 42.820 | 37.796 | 38.012 | | | 1.00 | 113.19 | | A | S |
| ANISOU | 54 | SG | CYS A | 50 | 13708 | 11276 | 18022 | −52 | −3216 | | | 3794 | A | S |
| ATOM | 55 | N | PRO A | 51 | 41.028 | 37.208 | 34.907 | | | 1.00 | 111.76 | | A | N |
| ANISOU | 55 | N | PRO A | 51 | 13379 | 11653 | 17432 | 474 | −3270 | | | 4214 | A | N |
| ATOM | 56 | CA | PRO A | 51 | 39.911 | 37.793 | 34.166 | | | 1.00 | 113.17 | | A | C |
| ANISOU | 56 | CA | PRO A | 51 | 13516 | 11829 | 17655 | 585 | −3332 | | | 4416 | A | C |
| ATOM | 57 | C | PRO A | 51 | 39.756 | 39.242 | 34.592 | | | 1.00 | 105.69 | | A | C |
| ANISOU | 57 | C | PRO A | 51 | 12623 | 10423 | 17113 | 568 | −3426 | | | 4447 | A | C |
| ATOM | 58 | O | PRO A | 51 | 39.514 | 39.517 | 35.764 | | | 1.00 | 98.68 | | A | O |
| ANISOU | 58 | O | PRO A | 51 | 11831 | 9213 | 16451 | 618 | −3441 | | | 4121 | A | O |
| ATOM | 59 | CB | PRO A | 51 | 38.718 | 36.977 | 34.648 | | | 1.00 | 118.11 | | A | C |
| ANISOU | 59 | CB | PRO A | 51 | 14160 | 12600 | 18115 | 806 | −3308 | | | 4130 | A | C |
| ATOM | 60 | CG | PRO A | 51 | 39.086 | 36.599 | 36.038 | | | 1.00 | 118.64 | | A | C |
| ANISOU | 60 | CG | PRO A | 51 | 14325 | 12479 | 18272 | 816 | −3271 | | | 3702 | A | C |
| ATOM | 61 | CD | PRO A | 51 | 40.568 | 36.342 | 36.004 | | | 1.00 | 117.49 | | A | C |
| ANISOU | 61 | CD | PRO A | 51 | 14183 | 12346 | 18114 | 611 | −3232 | | | 3770 | A | C |
| ATOM | 62 | N | VAL A | 52 | 39.897 | 40.159 | 33.645 | | | 1.00 | 116.60 | | A | N |
| ANISOU | 62 | N | VAL A | 52 | 13941 | 11785 | 18577 | 498 | −3487 | | | 4833 | A | N |
| ATOM | 63 | CA | VAL A | 52 | 39.805 | 41.578 | 33.949 | | | 1.00 | 117.67 | | A | C |
| ANISOU | 63 | CA | VAL A | 52 | 14126 | 11488 | 19094 | 454 | −3580 | | | 4905 | A | C |
| ATOM | 64 | C | VAL A | 52 | 38.391 | 42.137 | 33.940 | | | 1.00 | 122.26 | | A | C |
| ANISOU | 64 | C | VAL A | 52 | 14743 | 11887 | 19825 | 673 | −3636 | | | 4811 | A | C |
| ATOM | 65 | O | VAL A | 52 | 38.127 | 43.157 | 34.571 | | | 1.00 | 120.01 | | A | O |
| ANISOU | 65 | O | VAL A | 52 | 14537 | 11203 | 19857 | 700 | −3691 | | | 4683 | A | O |
| ATOM | 66 | CB | VAL A | 52 | 40.659 | 42.406 | 32.975 | | | 1.00 | 116.82 | | A | C |
| ANISOU | 66 | CB | VAL A | 52 | 13934 | 11432 | 19022 | 267 | −3627 | | | 5366 | A | C |
| ATOM | 67 | CG1 | VAL A | 52 | 40.126 | 43.825 | 32.878 | | | 1.00 | 118.92 | | A | C |
| ANISOU | 67 | CG1 | VAL A | 52 | 14256 | 11249 | 19680 | 218 | −3730 | | | 5446 | A | C |
| ATOM | 68 | CG2 | VAL A | 52 | 42.116 | 42.396 | 33.409 | | | 1.00 | 109.61 | | A | C |
| ANISOU | 68 | CG2 | VAL A | 52 | 12973 | 10720 | 17953 | 53 | −3562 | | | 5448 | A | C |
| ATOM | 69 | N | ASP A | 53 | 37.478 | 41.484 | 33.230 | | | 1.00 | 118.46 | | A | N |
| ANISOU | 69 | N | ASP A | 53 | 14200 | 11702 | 19106 | 835 | −3620 | | | 4860 | A | N |
| ATOM | 70 | CA | ASP A | 53 | 36.112 | 42.001 | 33.169 | | | 1.00 | 124.86 | | A | C |
| ANISOU | 70 | CA | ASP A | 53 | 15021 | 12394 | 20024 | 1052 | −3671 | | | 4808 | A | C |
| ATOM | 71 | C | ASP A | 53 | 35.126 | 41.211 | 34.021 | | | 1.00 | 117.67 | | A | C |
| ANISOU | 71 | C | ASP A | 53 | 14162 | 11498 | 19050 | 1236 | −3622 | | | 4372 | A | C |
| ATOM | 72 | O | ASP A | 53 | 33.916 | 41.293 | 33.794 | | | 1.00 | 110.53 | | A | O |
| ANISOU | 72 | O | ASP A | 53 | 13233 | 10639 | 18122 | 1433 | −3644 | | | 4330 | A | O |
| ATOM | 73 | CB | ASP A | 53 | 35.624 | 42.040 | 31.724 | | | 1.00 | 139.47 | | A | C |
| ANISOU | 73 | CB | ASP A | 53 | 16759 | 14568 | 21665 | 1114 | −3699 | | | 5161 | A | C |
| ATOM | 74 | CG | ASP A | 53 | 36.545 | 42.840 | 30.834 | | | 1.00 | 155.01 | | A | C |
| ANISOU | 74 | CG | ASP A | 53 | 18664 | 16546 | 23688 | 929 | −3746 | | | 5602 | A | C |
| ATOM | 75 | OD1 | ASP A | 53 | 37.265 | 43.718 | 31.374 | | | 1.00 | 159.96 | | A | O |
| ANISOU | 75 | OD1 | ASP A | 53 | 19345 | 16824 | 24608 | 799 | −3791 | | | 5646 | A | O |
| ATOM | 76 | OD2 | ASP A | 53 | 36.550 | 42.588 | 29.603 | | | 1.00 | 160.52 | | A | O1− |
| ANISOU | 76 | OD2 | ASP A | 53 | 19253 | 17611 | 24126 | 906 | −3738 | | | 5895 | A | O1− |
| ATOM | 77 | N | SER A | 54 | 35.610 | 40.452 | 34.990 | | | 1.00 | 120.11 | | A | N |
| ANISOU | 77 | N | SER A | 54 | 14531 | 11779 | 19326 | 1172 | −3556 | | | 4053 | A | N |
| ATOM | 78 | CA | SER A | 54 | 34.790 | 39.596 | 35.836 | | | 1.00 | 123.38 | | A | C |
| ANISOU | 78 | CA | SER A | 54 | 14987 | 12234 | 19659 | 1316 | −3500 | | | 3629 | A | C |
| ATOM | 79 | C | SER A | 54 | 35.259 | 39.713 | 37.272 | | | 1.00 | 125.41 | | A | C |
| ANISOU | 79 | C | SER A | 54 | 15348 | 12172 | 20129 | 1235 | −3474 | | | 3284 | A | C |
| ATOM | 80 | O | SER A | 54 | 36.319 | 40.283 | 37.552 | | | 1.00 | 125.91 | | A | O |
| ANISOU | 80 | O | SER A | 54 | 15445 | 12038 | 20357 | 1058 | −3494 | | | 3378 | A | O |
| ATOM | 81 | CB | SER A | 54 | 34.856 | 38.128 | 35.376 | | | 1.00 | 119.00 | | A | C |
| ANISOU | 81 | CB | SER A | 54 | 14380 | 12123 | 18712 | 1326 | −3429 | | | 3574 | A | C |
| ATOM | 82 | OG | SER A | 54 | 34.328 | 37.982 | 34.070 | | | 1.00 | 118.74 | | A | O |
| ANISOU | 82 | OG | SER A | 54 | 14247 | 12423 | 18445 | 1404 | −3447 | | | 3853 | A | O |
| ATOM | 83 | N | PRO A | 55 | 34.482 | 39.189 | 38.219 | | | 1.00 | 130.50 | | A | N |
| ANISOU | 83 | N | PRO A | 55 | 16040 | 12781 | 20764 | 1353 | −3428 | | | 2874 | A | N |
| ATOM | 84 | CA | PRO A | 55 | 34.996 | 39.044 | 39.577 | | | 1.00 | 129.39 | | A | C |
| ANISOU | 84 | CA | PRO A | 55 | 15988 | 12434 | 20741 | 1257 | −3383 | | | 2517 | A | C |
| ATOM | 85 | C | PRO A | 55 | 36.197 | 38.126 | 39.585 | | | 1.00 | 123.67 | | A | C |
| ANISOU | 85 | C | PRO A | 55 | 15261 | 11884 | 19842 | 1090 | −3336 | | | 2530 | A | C |
| ATOM | 86 | O | PRO A | 55 | 36.288 | 37.184 | 38.775 | | | 1.00 | 126.40 | | A | O |
| ANISOU | 86 | O | PRO A | 55 | 15546 | 12573 | 19906 | 1106 | −3308 | | | 2660 | A | O |
| ATOM | 87 | CB | PRO A | 55 | 33.813 | 38.431 | 40.342 | | | 1.00 | 128.42 | | A | C |
| ANISOU | 87 | CB | PRO A | 55 | 15885 | 12361 | 20548 | 1424 | −3330 | | | 2112 | A | C |
| ATOM | 88 | CG | PRO A | 55 | 32.901 | 37.892 | 39.286 | | | 1.00 | 128.89 | | A | C |
| ANISOU | 88 | CG | PRO A | 55 | 15858 | 12736 | 20380 | 1582 | −3339 | | | 2264 | A | C |
| ATOM | 89 | CD | PRO A | 55 | 33.062 | 38.817 | 38.131 | | | 1.00 | 130.49 | | A | C |
| ANISOU | 89 | CD | PRO A | 55 | 16004 | 12921 | 20654 | 1573 | −3416 | | | 2719 | A | C |
| ATOM | 90 | N | PRO A | 56 | 37.157 | 38.366 | 40.475 | | | 1.00 | 118.72 | | A | N |
| ANISOU | 90 | N | PRO A | 56 | 14695 | 11054 | 19359 | 929 | −3325 | | | 2403 | A | N |
| ATOM | 91 | CA | PRO A | 56 | 38.400 | 37.588 | 40.446 | | | 1.00 | 108.59 | | A | C |
| ANISOU | 91 | CA | PRO A | 56 | 13402 | 9928 | 17929 | 763 | −3286 | | | 2460 | A | C |
| ATOM | 92 | C | PRO A | 56 | 38.343 | 36.302 | 41.258 | | | 1.00 | 102.85 | | A | C |
| ANISOU | 92 | C | PRO A | 56 | 12713 | 9342 | 17023 | 789 | −3204 | | | 2076 | A | C |
| ATOM | 93 | O | PRO A | 56 | 39.000 | 35.326 | 40.882 | | | 1.00 | 101.03 | | A | O |
| ANISOU | 93 | O | PRO A | 56 | 12454 | 9360 | 16571 | 734 | −3165 | | | 2148 | A | O |
| ATOM | 94 | CB | PRO A | 56 | 39.426 | 38.573 | 41.013 | | | 1.00 | 106.15 | | A | C |
| ANISOU | 94 | CB | PRO A | 56 | 13131 | 9329 | 17871 | 577 | −3322 | | | 2519 | A | C |
| ATOM | 95 | CG | PRO A | 56 | 38.631 | 39.431 | 41.954 | | | 1.00 | 108.06 | | A | C |
| ANISOU | 95 | CG | PRO A | 56 | 13441 | 9272 | 18345 | 663 | −3339 | | | 2249 | A | C |
| ATOM | 96 | CD | PRO A | 56 | 37.222 | 39.496 | 41.421 | | | 1.00 | 113.49 | | A | C |
| ANISOU | 96 | CD | PRO A | 56 | 14102 | 10022 | 18998 | 884 | −3355 | | | 2260 | A | C |
| ATOM | 97 | N | SER A | 57 | 37.572 | 36.266 | 42.350 | | | 1.00 | 94.60 | | A | N |
| ANISOU | 97 | N | SER A | 57 | 11727 | 8168 | 16048 | 871 | −3171 | | | 1675 | A | N |
| ATOM | 98 | CA | SER A | 57 | 37.539 | 35.116 | 43.241 | | | 1.00 | 87.59 | | A | C |
| ANISOU | 98 | CA | SER A | 57 | 10879 | 7404 | 15000 | 875 | −3089 | | | 1290 | A | C |
| ATOM | 99 | C | SER A | 57 | 36.227 | 34.364 | 43.073 | | | 1.00 | 79.26 | | A | C |
| ANISOU | 99 | C | SER A | 57 | 9804 | 6543 | 13768 | 1064 | −3061 | | | 1108 | A | C |
| ATOM | 100 | O | SER A | 57 | 35.162 | 34.981 | 42.975 | | | 1.00 | 89.94 | | A | O |
| ANISOU | 100 | O | SER A | 57 | 11141 | 7819 | 15214 | 1193 | −3089 | | | 1097 | A | O |
| ATOM | 101 | CB | SER A | 57 | 37.710 | 35.542 | 44.692 | | | 1.00 | 91.40 | | A | C |
| ANISOU | 101 | CB | SER A | 57 | 11426 | 7674 | 15629 | 798 | −3052 | | | 955 | A | C |
| ATOM | 102 | OG | SER A | 57 | 38.093 | 34.435 | 45.490 | | | 1.00 | 92.41 | | A | O |
| ANISOU | 102 | OG | SER A | 57 | 11581 | 7949 | 15581 | 742 | −2969 | | | 660 | A | O |
| ATOM | 103 | N | CYS A | 58 | 36.357 | 33.055 | 42.866 | | | 1.00 | 68.90 | | A | N |
| ANISOU | 103 | N | CYS A | 58 | 8492 | 5487 | 12201 | 1080 | −3006 | | | 961 | A | N |
| ATOM | 104 | CA | CYS A | 58 | 35.226 | 32.186 | 42.590 | | | 1.00 | 72.63 | | A | C |
| ANISOU | 104 | CA | CYS A | 58 | 8925 | 6227 | 12445 | 1243 | −2991 | | | 883 | A | C |
| ATOM | 105 | C | CYS A | 58 | 35.514 | 30.734 | 42.965 | | | 1.00 | 61.26 | | A | C |
| ANISOU | 105 | C | CYS A | 58 | 7515 | 5006 | 10756 | 1224 | −2921 | | | 627 | A | C |
| ATOM | 106 | O | CYS A | 58 | 36.592 | 30.418 | 43.450 | | | 1.00 | 56.36 | | A | O |
| ANISOU | 106 | O | CYS A | 58 | 6931 | 4354 | 10129 | 1096 | −2892 | | | 599 | A | O |
| ATOM | 107 | CB | CYS A | 58 | 34.888 | 32.277 | 41.093 | | | 1.00 | 85.20 | | A | C |
| ANISOU | 107 | CB | CYS A | 58 | 10428 | 8024 | 13921 | 1304 | −3045 | | | 1309 | A | C |
| ATOM | 108 | SG | CYS A | 58 | 35.994 | 31.399 | 39.947 | | | 1.00 | 103.73 | | A | S |
| ANISOU | 108 | SG | CYS A | 58 | 12751 | 10337 | 16325 | 1115 | −3076 | | | 1740 | A | S |
| ATOM | 109 | N | GLU A | 59 | 34.539 | 29.858 | 42.742 | | | 1.00 | 60.93 | | A | N |
| ANISOU | 109 | N | GLU A | 59 | 7452 | 5194 | 10503 | 1352 | −2896 | | | 445 | A | N |
| ATOM | 110 | CA | GLU A | 59 | 34.692 | 28.438 | 43.035 | | | 1.00 | 52.11 | | A | C |
| ANISOU | 110 | CA | GLU A | 59 | 6368 | 4304 | 9129 | 1349 | −2831 | | | 184 | A | C |
| ATOM | 111 | C | GLU A | 59 | 34.633 | 27.639 | 41.738 | | | 1.00 | 47.29 | | A | C |
| ANISOU | 111 | C | GLU A | 59 | 5684 | 4068 | 8215 | 1420 | −2850 | | | 397 | A | C |
| ATOM | 112 | O | GLU A | 59 | 33.701 | 27.807 | 40.946 | | | 1.00 | 52.46 | | A | O |
| ANISOU | 112 | O | GLU A | 59 | 6268 | 4876 | 8788 | 1525 | −2886 | | | 529 | A | O |
| ATOM | 113 | CB | GLU A | 59 | 33.606 | 27.967 | 44.008 | | | 1.00 | 46.43 | | A | C |
| ANISOU | 113 | CB | GLU A | 59 | 5685 | 3598 | 8360 | 1403 | −2773 | | | −244 | A | C |
| ATOM | 114 | CG | GLU A | 59 | 33.389 | 28.875 | 45.241 | | | 1.00 | 60.21 | | A | C |
| ANISOU | 114 | CG | GLU A | 59 | 7464 | 5073 | 10340 | 1334 | −2749 | | | −438 | A | C |
| ATOM | 115 | CD | GLU A | 59 | 34.557 | 28.856 | 46.271 | | | 1.00 | 75.79 | | A | C |
| ANISOU | 115 | CD | GLU A | 59 | 9490 | 6941 | 12367 | 1157 | −2689 | | | −564 | A | C |
| ATOM | 116 | OE1 | GLU A | 59 | 35.364 | 27.889 | 46.275 | | | 1.00 | 76.96 | | A | O |
| ANISOU | 116 | OE1 | GLU A | 59 | 9668 | 7219 | 12355 | 1097 | −2646 | | | −613 | A | O |
| ATOM | 117 | OE2 | GLU A | 59 | 34.659 | 29.813 | 47.091 | | | 1.00 | 79.30 | | A | O1− |
| ANISOU | 117 | OE2 | GLU A | 59 | 9937 | 7199 | 12994 | 1089 | −2680 | | | −609 | A | O1− |
| ATOM | 118 | N | THR A | 60 | 35.623 | 26.779 | 41.521 | | | 1.00 | 45.71 | | A | N |
| ANISOU | 118 | N | THR A | 60 | 5493 | 4048 | 7825 | 1355 | −2822 | | | 430 | A | N |
| ATOM | 119 | CA | THR A | 60 | 35.731 | 26.019 | 40.266 | | | 1.00 | 52.03 | | A | C |
| ANISOU | 119 | CA | THR A | 60 | 6231 | 5286 | 8252 | 1352 | −2770 | | | 620 | A | C |
| ATOM | 120 | C | THR A | 60 | 36.275 | 24.623 | 40.587 | | | 1.00 | 42.81 | | A | C |
| ANISOU | 120 | C | THR A | 60 | 5128 | 4362 | 6776 | 1277 | −2612 | | | 345 | A | C |
| ATOM | 121 | O | THR A | 60 | 36.201 | 24.161 | 41.730 | | | 1.00 | 40.64 | | A | O |
| ANISOU | 121 | O | THR A | 60 | 4934 | 3952 | 6555 | 1266 | −2565 | | | −14 | A | O |
| ATOM | 122 | CB | THR A | 60 | 36.581 | 26.793 | 39.229 | | | 1.00 | 48.35 | | A | C |
| ANISOU | 122 | CB | THR A | 60 | 5689 | 4858 | 7824 | 1278 | −2816 | | | 1111 | A | C |
| ATOM | 123 | OG1 | THR A | 60 | 36.518 | 26.138 | 37.954 | | | 1.00 | 48.75 | | A | O |
| ANISOU | 123 | OG1 | THR A | 60 | 5668 | 5355 | 7499 | 1287 | −2764 | | | 1294 | A | O |
| ATOM | 124 | CG2 | THR A | 60 | 38.024 | 26.915 | 39.669 | | | 1.00 | 47.96 | | A | C |
| ANISOU | 124 | CG2 | THR A | 60 | 5677 | 4680 | 7865 | 1117 | −2761 | | | 1178 | A | C |
| ATOM | 125 | N | THR A | 61 | 36.793 | 23.927 | 39.578 | | | 1.00 | 42.76 | | A | N |
| ANISOU | 125 | N | THR A | 61 | 5085 | 4722 | 6440 | 1233 | −2528 | | | 506 | A | N |
| ATOM | 126 | CA | THR A | 61 | 37.179 | 22.539 | 39.764 | | | 1.00 | 40.22 | | A | C |
| ANISOU | 126 | CA | THR A | 61 | 4826 | 4648 | 5808 | 1190 | −2378 | | | 243 | A | C |
| ATOM | 127 | C | THR A | 61 | 38.529 | 22.289 | 39.110 | | | 1.00 | 40.64 | | A | C |
| ANISOU | 127 | C | THR A | 61 | 4849 | 4896 | 5696 | 1087 | −2289 | | | 482 | A | C |
| ATOM | 128 | O | THR A | 61 | 38.967 | 23.032 | 38.240 | | | 1.00 | 43.93 | | A | O |
| ANISOU | 128 | O | THR A | 61 | 5181 | 5363 | 6147 | 1056 | −2338 | | | 866 | A | O |
| ATOM | 129 | CB | THR A | 61 | 36.124 | 21.571 | 39.203 | | | 1.00 | 39.50 | | A | C |
| ANISOU | 129 | CB | THR A | 61 | 4734 | 4882 | 5394 | 1272 | −2342 | | | 75 | A | C |
| ATOM | 130 | CG | THR A | 61 | 34.859 | 21.547 | 40.017 | | | 1.00 | 42.21 | | A | C |
| ANISOU | 130 | CG | THR A | 61 | 5109 | 5074 | 5854 | 1360 | −2400 | | | −233 | A | C |
| ATOM | 131 | CD1 | THR A | 61 | 33.833 | 22.463 | 39.791 | | | 1.00 | 48.15 | | A | C |
| ANISOU | 131 | CD1 | THR A | 61 | 5789 | 5717 | 6788 | 1464 | −2533 | | | −114 | A | C |
| ATOM | 132 | CD2 | THR A | 61 | 34.690 | 20.619 | 41.019 | | | 1.00 | 43.46 | | A | C |
| ANISOU | 132 | CD2 | THR A | 61 | 5362 | 5206 | 5945 | 1343 | −2321 | | | −634 | A | C |
| ATOM | 133 | CE1 | THR A | 61 | 32.667 | 22.448 | 40.565 | | | 1.00 | 50.86 | | A | C |
| ANISOU | 133 | CE1 | THR A | 61 | 6149 | 5938 | 7239 | 1554 | −2580 | | | −403 | A | C |
| ATOM | 134 | CE2 | THR A | 61 | 33.542 | 20.594 | 41.797 | | | 1.00 | 42.89 | | A | C |
| ANISOU | 134 | CE2 | THR A | 61 | 5309 | 5015 | 5972 | 1416 | −2369 | | | −917 | A | C |
| ATOM | 135 | CZ | THR A | 61 | 32.535 | 21.497 | 41.581 | | | 1.00 | 48.98 | | A | C |
| ANISOU | 135 | CZ | THR A | 61 | 6003 | 5690 | 6918 | 1523 | −2494 | | | −811 | A | C |
| ATOM | 136 | OH | THR A | 61 | 31.418 | 21.414 | 42.405 | | | 1.00 | 45.79 | | A | O |
| ANISOU | 136 | OH | THR A | 61 | 5609 | 5189 | 6600 | 1599 | −2529 | | | −1112 | A | O |
| ATOM | 137 | N | VAL A | 62 | 39.182 | 21.228 | 39.552 | | | 1.00 | 38.47 | | A | N |
| ANISOU | 137 | N | VAL A | 62 | 4641 | 4735 | 5242 | 1038 | −2156 | | | 254 | A | N |
| ATOM | 138 | CA | VAL A | 62 | 40.508 | 20.850 | 39.098 | | | 1.00 | 38.61 | | A | C |
| ANISOU | 138 | CA | VAL A | 62 | 4633 | 4942 | 5096 | 953 | −2050 | | | 421 | A | C |
| ATOM | 139 | C | VAL A | 62 | 40.444 | 19.365 | 38.774 | | | 1.00 | 43.15 | | A | C |
| ANISOU | 139 | C | VAL A | 62 | 5257 | 5854 | 5282 | 986 | −1909 | | | 191 | A | C |
| ATOM | 140 | O | VAL A | 62 | 39.796 | 18.599 | 39.494 | | | 1.00 | 42.73 | | A | O |
| ANISOU | 140 | O | VAL A | 62 | 5294 | 5762 | 5178 | 1025 | −1878 | | | −168 | A | O |
| ATOM | 141 | CB | VAL A | 62 | 41.567 | 21.143 | 40.184 | | | 1.00 | 37.82 | | A | C |
| ANISOU | 141 | CB | VAL A | 62 | 4566 | 4571 | 5233 | 852 | −2033 | | | 371 | A | C |
| ATOM | 142 | CG1 | VAL A | 62 | 42.600 | 20.052 | 40.238 | | | 1.00 | 36.37 | | A | C |
| ANISOU | 142 | CG1 | VAL A | 62 | 4406 | 4604 | 4808 | 806 | −1876 | | | 275 | A | C |
| ATOM | 143 | CG2 | VAL A | 62 | 42.195 | 22.513 | 39.992 | | | 1.00 | 40.26 | | A | C |
| ANISOU | 143 | CG2 | VAL A | 62 | 4800 | 4674 | 5823 | 772 | −2134 | | | 742 | A | C |
| ATOM | 144 | N | ALA A | 63 | 41.084 | 18.943 | 37.689 | | | 1.00 | 37.91 | | A | N |
| ANISOU | 144 | N | ALA A | 63 | 4540 | 5523 | 4340 | 971 | −1824 | | | 389 | A | N |
| ATOM | 145 | CA | ALA A | 63 | 40.997 | 17.540 | 37.298 | | | 1.00 | 36.64 | | A | C |
| ANISOU | 145 | CA | ALA A | 63 | 4436 | 5679 | 3807 | 1009 | −1691 | | | 167 | A | C |
| ATOM | 146 | C | ALA A | 63 | 42.219 | 16.762 | 37.774 | | | 1.00 | 35.29 | | A | C |
| ANISOU | 146 | C | ALA A | 63 | 4308 | 5545 | 3554 | 965 | −1551 | | | 55 | A | C |
| ATOM | 147 | O | ALA A | 63 | 43.356 | 17.193 | 37.580 | | | 1.00 | 36.35 | | A | O |
| ANISOU | 147 | O | ALA A | 63 | 4370 | 5696 | 3743 | 903 | −1522 | | | 305 | A | O |
| ATOM | 148 | CB | ALA A | 63 | 40.833 | 17.397 | 35.785 | | | 1.00 | 38.60 | | A | C |
| ANISOU | 148 | CB | ALA A | 63 | 4606 | 6310 | 3750 | 1036 | −1670 | | | 399 | A | C |
| ATOM | 149 | N | THR A | 64 | 41.975 | 15.599 | 38.362 | | | 1.00 | 33.11 | | A | N |
| ANISOU | 149 | N | THR A | 64 | 4144 | 5295 | 3141 | 997 | −1464 | | | −309 | A | N |
| ATOM | 150 | CA | THR A | 64 | 42.981 | 14.803 | 39.034 | | | 1.00 | 31.59 | | A | C |
| ANISOU | 150 | CA | THR A | 64 | 4008 | 5088 | 2906 | 973 | −1342 | | | −467 | A | C |
| ATOM | 151 | C | THR A | 64 | 42.871 | 13.361 | 38.587 | | | 1.00 | 30.73 | | A | C |
| ANISOU | 151 | C | THR A | 64 | 3980 | 5258 | 2440 | 1031 | −1207 | | | −692 | A | C |
| ATOM | 152 | O | THR A | 64 | 41.800 | 12.882 | 38.238 | | | 1.00 | 36.32 | | A | O |
| ANISOU | 152 | O | THR A | 64 | 4739 | 6072 | 2988 | 1075 | −1224 | | | −850 | A | O |
| ATOM | 153 | CB | THR A | 64 | 42.805 | 14.863 | 40.542 | | | 1.00 | 29.64 | | A | C |
| ANISOU | 153 | CB | THR A | 64 | 3839 | 4503 | 2920 | 944 | −1384 | | | −725 | A | C |
| ATOM | 154 | CG | THR A | 64 | 44.008 | 14.414 | 41.308 | | | 1.00 | 28.55 | | A | C |
| ANISOU | 154 | CG | THR A | 64 | 3724 | 4304 | 2819 | 898 | −1291 | | | −793 | A | C |
| ATOM | 155 | CD1 | THR A | 64 | 44.259 | 13.067 | 41.527 | | | 1.00 | 27.03 | | A | C |
| ANISOU | 155 | CD1 | THR A | 64 | 3620 | 4244 | 2407 | 937 | −1161 | | | −1041 | A | C |
| ATOM | 156 | CD2 | THR A | 64 | 44.907 | 15.346 | 41.830 | | | 1.00 | 29.18 | | A | C |
| ANISOU | 156 | CD2 | THR A | 64 | 3736 | 4188 | 3161 | 814 | −1339 | | | −603 | A | C |
| ATOM | 157 | CE1 | THR A | 64 | 45.391 | 12.666 | 42.225 | | | 1.00 | 26.20 | | A | C |
| ANISOU | 157 | CE1 | THR A | 64 | 3523 | 4091 | 2341 | 905 | −1079 | | | −1082 | A | C |
| ATOM | 158 | CE2 | THR A | 64 | 46.007 | 14.952 | 42.521 | | | 1.00 | 28.32 | | A | C |
| ANISOU | 158 | CE2 | THR A | 64 | 3633 | 4046 | 3081 | 768 | −1260 | | | −652 | A | C |
| ATOM | 159 | CZ | THR A | 64 | 46.254 | 13.613 | 42.720 | | | 1.00 | 26.82 | | A | C |
| ANISOU | 159 | CZ | THR A | 64 | 3520 | 4002 | 2670 | 820 | −1130 | | | −886 | A | C |
| ATOM | 160 | OH | THR A | 64 | 47.381 | 13.238 | 43.420 | | | 1.00 | 26.12 | | A | O |
| ANISOU | 160 | OH | THR A | 64 | 3425 | 3884 | 2617 | 783 | −1055 | | | −914 | A | O |
| ATOM | 161 | N | ARG A | 65 | 43.984 | 12.662 | 38.620 | | | 1.00 | 30.38 | | A | N |
| ANISOU | 161 | N | ARG A | 65 | 3946 | 5323 | 2273 | 1031 | −1075 | | | −710 | A | N |
| ATOM | 162 | CA | ARG A | 65 | 44.068 | 11.316 | 38.084 | | | 1.00 | 33.86 | | A | C |
| ANISOU | 162 | CA | ARG A | 65 | 4446 | 5955 | 2464 | 1054 | −889 | | | −855 | A | C |
| ATOM | 163 | C | ARG A | 65 | 44.681 | 10.402 | 39.134 | | | 1.00 | 41.31 | | A | C |
| ANISOU | 163 | C | ARG A | 65 | 5451 | 6680 | 3567 | 1006 | −743 | | | −1025 | A | C |
| ATOM | 164 | O | ARG A | 65 | 45.781 | 10.668 | 39.626 | | | 1.00 | 51.64 | | A | O |
| ANISOU | 164 | O | ARG A | 65 | 6736 | 8007 | 4877 | 1031 | −767 | | | −989 | A | O |
| ATOM | 165 | CB | ARG A | 65 | 44.889 | 11.309 | 36.788 | | | 1.00 | 42.50 | | A | C |
| ANISOU | 165 | CB | ARG A | 65 | 5448 | 7410 | 3290 | 1089 | −824 | | | −606 | A | C |
| ATOM | 166 | CG | ARG A | 65 | 44.085 | 11.668 | 35.528 | | | 1.00 | 34.03 | | A | C |
| ANISOU | 166 | CG | ARG A | 65 | 4317 | 6541 | 2072 | 1091 | −869 | | | −439 | A | C |
| ATOM | 167 | CD | ARG A | 65 | 44.997 | 12.183 | 34.411 | | | 1.00 | 40.56 | | A | C |
| ANISOU | 167 | CD | ARG A | 65 | 5004 | 7694 | 2713 | 1101 | −856 | | | −77 | A | C |
| ATOM | 168 | NE | ARG A | 65 | 44.226 | 12.503 | 33.222 | | | 1.00 | 52.64 | | A | N |
| ANISOU | 168 | NE | ARG A | 65 | 6475 | 9397 | 4128 | 1085 | −889 | | | 83 | A | N |
| ATOM | 169 | CZ | ARG A | 65 | 43.484 | 11.604 | 32.576 | | | 1.00 | 68.98 | | A | C |
| ANISOU | 169 | CZ | ARG A | 65 | 8607 | 11503 | 6098 | 1066 | −780 | | | −102 | A | C |
| ATOM | 170 | NH1 | ARG A | 65 | 43.423 | 10.355 | 33.030 | | | 1.00 | 66.13 | | A | N1+ |
| ANISOU | 170 | NH1 | ARG A | 65 | 8358 | 10972 | 5797 | 1048 | −639 | | | −406 | A | N1+ |
| ATOM | 171 | NH2 | ARG A | 65 | 42.794 | 11.941 | 31.489 | | | 1.00 | 79.93 | | A | N |
| ANISOU | 171 | NH2 | ARG A | 65 | 9933 | 13063 | 7371 | 1049 | −822 | | | 48 | A | N |
| ATOM | 172 | N | ALA A | 66 | 43.962 | 9.339 | 39.487 | | | 1.00 | 43.87 | | A | N |
| ANISOU | 172 | N | ALA A | 66 | 7435 | 3386 | 5848 | −754 | 216 | | | −677 | A | N |
| ATOM | 173 | CA | ALA A | 66 | 44.450 | 8.408 | 40.490 | | | 1.00 | 45.88 | | A | C |
| ANISOU | 173 | CA | ALA A | 66 | 7716 | 3568 | 6149 | −692 | 181 | | | −669 | A | C |
| ATOM | 174 | C | ALA A | 66 | 45.790 | 7.821 | 40.058 | | | 1.00 | 53.76 | | A | C |
| ANISOU | 174 | C | ALA A | 66 | 8782 | 4455 | 7189 | −527 | 228 | | | −711 | A | C |
| ATOM | 175 | O | ALA A | 66 | 45.982 | 7.460 | 38.893 | | | 1.00 | 59.13 | | A | O |
| ANISOU | 175 | O | ALA A | 66 | 9596 | 5089 | 7780 | −507 | 285 | | | −769 | A | O |
| ATOM | 176 | CB | ALA A | 66 | 43.419 | 7.300 | 40.716 | | | 1.00 | 40.04 | | A | C |
| ANISOU | 176 | CB | ALA A | 66 | 7103 | 2812 | 5296 | −851 | 128 | | | −672 | A | C |
| ATOM | 177 | N | GLN A | 67 | 46.727 | 7.818 | 41.001 | | | 1.00 | 47.91 | | A | N |
| ANISOU | 177 | N | GLN A | 67 | 7951 | 3671 | 6580 | −404 | 204 | | | −680 | A | N |
| ATOM | 178 | CA | GLN A | 67 | 48.086 | 7.348 | 40.794 | | | 1.00 | 44.34 | | A | C |
| ANISOU | 178 | CA | GLN A | 67 | 7515 | 3123 | 6210 | −232 | 245 | | | −700 | A | C |
| ATOM | 179 | C | GLN A | 67 | 48.243 | 5.998 | 41.449 | | | 1.00 | 47.46 | | A | C |
| ANISOU | 179 | C | GLN A | 67 | 8003 | 3422 | 6607 | −216 | 207 | | | −720 | A | C |
| ATOM | 180 | O | GLN A | 67 | 47.653 | 5.733 | 42.485 | | | 1.00 | 39.50 | | A | O |
| ANISOU | 18C | O | GLN A | 67 | 6979 | 2427 | 5604 | −294 | 142 | | | −686 | A | O |
| ATOM | 181 | CB | GLN A | 67 | 49.094 | 8.336 | 41.366 | | | 1.00 | 42.20 | | A | C |
| ANISOU | 181 | CB | GLN A | 67 | 7045 | 2866 | 6123 | −99 | 227 | | | −639 | A | C |
| ATOM | 182 | CG | GLN A | 67 | 49.000 | 9.737 | 40.786 | | | 1.00 | 50.85 | | A | C |
| ANISOU | 182 | CG | GLN A | 67 | 8037 | 4032 | 7251 | −84 | 266 | | | −605 | A | C |
| ATOM | 183 | CD | GLN A | 67 | 49.956 | 10.708 | 41.451 | | | 1.00 | 62.56 | | A | C |
| ANISOU | 183 | CD | GLN A | 67 | 9331 | 5516 | 8923 | 40 | 195 | | | −528 | A | C |
| ATOM | 184 | OE1 | GLN A | 67 | 51.122 | 10.392 | 41.671 | | | 1.00 | 75.67 | | A | O |
| ANISOU | 184 | OE1 | GLN A | 67 | 10969 | 7105 | 10678 | 181 | 192 | | | −508 | A | O |
| ATOM | 185 | NE2 | GLN A | 67 | 49.466 | 11.894 | 41.773 | | | 1.00 | 61.83 | | A | N |
| ANISOU | 185 | NE2 | GLN A | 67 | 9119 | 5501 | 8872 | −10 | 118 | | | −484 | A | N |
| ATOM | 186 | N | SER A | 68 | 49.053 | 5.149 | 40.837 | | | 1.00 | 58.67 | | A | N |
| ANISOU | 186 | N | SER A | 68 | 9521 | 4740 | 8029 | −95 | 260 | | | −768 | A | N |
| ATOM | 187 | CA | SER A | 68 | 49.216 | 3.783 | 41.301 | | | 1.00 | 67.34 | | A | C |
| ANISOU | 187 | CA | SER A | 68 | 10770 | 5724 | 9091 | −86 | 238 | | | −810 | A | C |
| ATOM | 188 | C | SER A | 68 | 49.768 | 3.559 | 42.702 | | | 1.00 | 61.51 | | A | C |
| ANISOU | 188 | C | SER A | 68 | 9951 | 4947 | 8473 | −67 | 167 | | | −760 | A | C |
| ATOM | 189 | O | SER A | 68 | 49.242 | 2.730 | 43.436 | | | 1.00 | 71.08 | | A | O |
| ANISOU | 189 | O | SER A | 68 | 11287 | 6096 | 9624 | −145 | 124 | | | −771 | A | O |
| ATOM | 190 | CB | SER A | 68 | 50.118 | 3.031 | 40.314 | | | 1.00 | 78.31 | | A | C |
| ANISOU | 190 | CB | SER A | 68 | 12272 | 7013 | 10471 | 78 | 326 | | | −874 | A | C |
| ATOM | 191 | OG | SER A | 68 | 49.957 | 3.503 | 38.985 | | | 1.00 | 76.80 | | A | O |
| ANISOU | 191 | OG | SER A | 68 | 12219 | 6837 | 10123 | 59 | 405 | | | −934 | A | O |
| ATOM | 192 | N | PRO A | 69 | 50.846 | 4.252 | 43.073 | | | 1.00 | 60.66 | | A | N |
| ANISOU | 192 | N | PRO A | 69 | 9651 | 4861 | 8538 | 31 | 146 | | | −703 | A | N |
| ATOM | 193 | CA | PRO A | 69 | 51.344 | 3.980 | 44.427 | | | 1.00 | 52.69 | | A | C |
| ANISOU | 193 | CA | PRO A | 69 | 8586 | 3813 | 7619 | 31 | 83 | | | −658 | A | C |
| ATOM | 194 | C | PRO A | 69 | 50.578 | 4.510 | 45.650 | | | 1.00 | 51.56 | | A | C |
| ANISOU | 194 | C | PRO A | 69 | 8364 | 3770 | 7455 | −79 | 37 | | | −601 | A | C |
| ATOM | 195 | O | PRO A | 69 | 50.237 | 3.724 | 46.522 | | | 1.00 | 44.25 | | A | O |
| ANISOU | 195 | O | PRO A | 69 | 7544 | 2839 | 6429 | −190 | 8 | | | −588 | A | O |
| ATOM | 196 | CB | PRO A | 69 | 52.747 | 4.602 | 44.414 | | | 1.00 | 53.75 | | A | C |
| ANISOU | 196 | CB | PRO A | 69 | 8572 | 3895 | 7955 | 199 | 85 | | | −630 | A | C |
| ATOM | 197 | CG | PRO A | 69 | 53.025 | 4.983 | 42.997 | | | 1.00 | 54.85 | | A | C |
| ANISOU | 197 | CG | PRO A | 69 | 8614 | 4104 | 8122 | 255 | 112 | | | −622 | A | C |
| ATOM | 198 | CD | PRO A | 69 | 51.701 | 5.188 | 42.339 | | | 1.00 | 53.14 | | A | C |
| ANISOU | 198 | CD | PRO A | 69 | 8557 | 3927 | 7704 | 168 | 180 | | | −680 | A | C |
| ATOM | 199 | N | ASN A | 70 | 50.243 | 5.796 | 45.681 | | | 1.00 | 47.40 | | A | N |
| ANISOU | 199 | N | ASN A | 70 | 7668 | 3328 | 7013 | −47 | 32 | | | −566 | A | N |
| ATOM | 200 | CA | ASN A | 70 | 49.609 | 6.362 | 46.872 | | | 1.00 | 49.75 | | A | C |
| ANISOU | 200 | CA | ASN A | 70 | 7881 | 3701 | 7321 | −98 | −3 | | | −516 | A | C |
| ATOM | 201 | C | ASN A | 70 | 48.268 | 7.030 | 46.712 | | | 1.00 | 53.44 | | A | C |
| ANISOU | 201 | C | ASN A | 70 | 8381 | 4272 | 7652 | −228 | −5 | | | −513 | A | C |
| ATOM | 202 | O | ASN A | 70 | 47.674 | 7.441 | 47.696 | | | 1.00 | 56.95 | | A | O |
| ANISOU | 202 | O | ASN A | 70 | 8798 | 4768 | 8073 | −269 | −30 | | | −477 | A | O |
| ATOM | 203 | CB | ASN A | 70 | 50.524 | 7.421 | 47.486 | | | 1.00 | 53.92 | | A | C |
| ANISOU | 203 | CB | ASN A | 70 | 8208 | 4249 | 8029 | 6 | −1 | | | −478 | A | C |
| ATOM | 204 | CG | ASN A | 70 | 51.985 | 7.063 | 47.395 | | | 1.00 | 55.37 | | A | C |
| ANISOU | 204 | CG | ASN A | 70 | 8333 | 4348 | 8359 | 128 | 18 | | | −460 | A | C |
| ATOM | 205 | OD1 | ASN A | 70 | 52.468 | 6.200 | 48.114 | | | 1.00 | 56.47 | | A | O |
| ANISOU | 205 | OD1 | ASN A | 70 | 8371 | 4503 | 8583 | 220 | 23 | | | −446 | A | O |
| ATOM | 206 | ND2 | ASN A | 70 | 52.703 | 7.748 | 46.521 | | | 1.00 | 51.47 | | A | N |
| ANISOU | 206 | ND2 | ASN A | 70 | 7917 | 3766 | 7875 | 132 | 15 | | | −451 | A | N |
| ATOM | 207 | N | PHE A | 71 | 47.784 | 7.148 | 45.491 | | | 1.00 | 58.34 | | A | N |
| ANISOU | 207 | N | PHE A | 71 | 9064 | 4922 | 8181 | −289 | 28 | | | −548 | A | N |
| ATOM | 208 | CA | PHE A | 71 | 46.544 | 7.868 | 45.272 | | | 1.00 | 43.36 | | A | C |
| ANISOU | 208 | CA | PHE A | 71 | 7151 | 3129 | 6195 | −403 | 32 | | | −536 | A | C |
| ATOM | 209 | C | PHE A | 71 | 45.328 | 7.092 | 44.798 | | | 1.00 | 41.88 | | A | C |
| ANISOU | 209 | C | PHE A | 71 | 7120 | 2944 | 5848 | −559 | 47 | | | −559 | A | C |
| ATOM | 210 | O | PHE A | 71 | 44.518 | 7.608 | 44.047 | | | 1.00 | 45.19 | | A | O |
| ANISOU | 210 | O | PHE A | 71 | 7546 | 3425 | 6200 | −639 | 71 | | | −574 | A | O |
| ATOM | 211 | CB | PHE A | 71 | 46.809 | 9.130 | 44.466 | | | 1.00 | 34.21 | | A | C |
| ANISOU | 211 | CB | PHE A | 71 | 5866 | 2022 | 5109 | −340 | 51 | | | −538 | A | C |
| ATOM | 212 | CG | PHE A | 71 | 47.746 | 10.073 | 45.156 | | | 1.00 | 45.20 | | A | C |
| ANISOU | 212 | CG | PHE A | 71 | 7083 | 3419 | 6672 | −221 | 12 | | | −500 | A | C |
| ATOM | 213 | CD2 | PHE A | 71 | 49.109 | 9.890 | 45.083 | | | 1.00 | 44.16 | | A | C |
| ANISOU | 213 | CD2 | PHE A | 71 | 6874 | 3215 | 6690 | −86 | 4 | | | −493 | A | C |
| ATOM | 214 | CD1 | PHE A | 71 | 47.262 | 11.114 | 45.916 | | | 1.00 | 53.96 | | A | C |
| ANISOU | 214 | CD1 | PHE A | 71 | 8111 | 4595 | 7796 | −247 | −14 | | | −472 | A | C |
| ATOM | 215 | CE2 | PHE A | 71 | 49.972 | 10.745 | 45.730 | | | 1.00 | 37.67 | | A | C |
| ANISOU | 215 | CE2 | PHE A | 71 | 5874 | 2435 | 6005 | −1 | −10 | | | −438 | A | C |
| ATOM | 216 | CE1 | PHE A | 71 | 48.121 | 11.975 | 46.567 | | | 1.00 | 39.41 | | A | C |
| ANISOU | 216 | CE1 | PHE A | 71 | 6117 | 2756 | 6100 | −147 | −18 | | | −441 | A | C |
| ATOM | 217 | CZ | PHE A | 71 | 49.478 | 11.792 | 46.473 | | | 1.00 | 28.09 | | A | C |
| ANISOU | 217 | CZ | PHE A | 71 | 4595 | 1302 | 4774 | −39 | 17 | | | −413 | A | C |
| ATOM | 218 | N | LEU A | 72 | 45.232 | 5.836 | 45.207 | | | 1.00 | 39.64 | | A | N |
| ANISOU | 218 | N | LEU A | 72 | 6964 | 2591 | 5508 | −617 | 23 | | | −553 | A | N |
| ATOM | 219 | CA | LEU A | 72 | 44.085 | 5.014 | 44.845 | | | 1.00 | 43.24 | | A | C |
| ANISOU | 219 | CA | LEU A | 72 | 7562 | 3038 | 5828 | −789 | 9 | | | −555 | A | C |
| ATOM | 220 | C | LEU A | 72 | 42.904 | 5.133 | 45.806 | | | 1.00 | 51.37 | | A | C |
| ANISOU | 220 | C | LEU A | 72 | 8597 | 4125 | 6795 | −935 | −14 | | | −486 | A | C |
| ATOM | 221 | O | LEU A | 72 | 41.952 | 4.360 | 45.674 | | | 1.00 | 66.20 | | A | O |
| ANISOU | 221 | O | LEU A | 72 | 10584 | 5988 | 8581 | −1094 | −38 | | | −466 | A | O |
| ATOM | 222 | CB | LEU A | 72 | 44.480 | 3.539 | 44.769 | | | 1.00 | 44.76 | | A | C |
| ANISOU | 222 | CB | LEU A | 72 | 7911 | 3100 | 5997 | −781 | −14 | | | −583 | A | C |
| ATOM | 223 | CG | LEU A | 72 | 45.483 | 3.108 | 43.701 | | | 1.00 | 42.58 | | A | C |
| ANISOU | 223 | CG | LEU A | 72 | 7693 | 2741 | 5746 | −660 | 17 | | | −659 | A | C |
| ATOM | 224 | CD1 | LEU A | 72 | 45.339 | 3.996 | 42.513 | | | 1.00 | 42.61 | | A | C |
| ANISOU | 224 | CD1 | LEU A | 72 | 7659 | 2816 | 5716 | −656 | 64 | | | −695 | A | C |
| ATOM | 225 | CD2 | LEU A | 72 | 46.868 | 3.189 | 44.234 | | | 1.00 | 53.14 | | A | C |
| ANISOU | 225 | CD2 | LEU A | 72 | 8941 | 4026 | 7224 | −480 | 30 | | | −656 | A | C |
| ATOM | 226 | N | SER A | 73 | 42.930 | 6.042 | 46.782 | | | 1.00 | 46.27 | | A | N |
| ANISOU | 226 | N | SER A | 73 | 7851 | 3536 | 6194 | −889 | −10 | | | −445 | A | N |
| ATOM | 227 | CA | SER A | 73 | 41.781 | 6.273 | 47.641 | | | 1.00 | 32.79 | | A | C |
| ANISOU | 227 | CA | SER A | 73 | 6164 | 1882 | 4411 | −1025 | −12 | | | −380 | A | C |
| ATOM | 228 | C | SER A | 73 | 41.442 | 7.750 | 47.627 | | | 1.00 | 33.18 | | A | C |
| ANISOU | 228 | C | SER A | 73 | 6083 | 2038 | 4487 | −1009 | 13 | | | −375 | A | C |
| ATOM | 229 | O | SER A | 73 | 42.331 | 8.610 | 47.605 | | | 1.00 | 38.74 | | A | O |
| ANISOU | 229 | O | SER A | 73 | 6677 | 2752 | 5289 | −855 | 11 | | | −403 | A | O |
| ATOM | 230 | CB | SER A | 73 | 42.029 | 5.826 | 49.083 | | | 1.00 | 46.84 | | A | C |
| ANISOU | 230 | CB | SER A | 73 | 8013 | 3601 | 6182 | −996 | −34 | | | −330 | A | C |
| ATOM | 231 | OG | SER A | 73 | 42.698 | 6.829 | 49.822 | | | 1.00 | 44.92 | | A | O |
| ANISOU | 231 | OG | SER A | 73 | 7676 | 3381 | 6010 | −853 | −38 | | | −332 | A | O |
| ATOM | 232 | N | LEU A | 74 | 40.142 | 8.039 | 47.658 | | | 1.00 | 31.62 | | A | N |
| ANISOU | 232 | N | LEU A | 74 | 5888 | 1915 | 4210 | −1178 | 32 | | | −333 | A | N |
| ATOM | 233 | CA | LEU A | 74 | 39.715 | 9.428 | 47.794 | | | 1.00 | 35.61 | | A | C |
| ANISOU | 233 | CA | LEU A | 74 | 6284 | 2512 | 4736 | −1174 | 58 | | | −325 | A | C |
| ATOM | 234 | C | LEU A | 74 | 40.224 | 10.065 | 49.090 | | | 1.00 | 42.75 | | A | C |
| ANISOU | 234 | C | LEU A | 74 | 7185 | 3385 | 5672 | −1044 | 51 | | | −314 | A | C |
| ATOM | 235 | O | LEU A | 74 | 40.401 | 11.288 | 49.154 | | | 1.00 | 46.89 | | A | O |
| ANISOU | 235 | O | LEU A | 74 | 7613 | 3947 | 6255 | −954 | 49 | | | −336 | A | O |
| ATOM | 236 | CB | LEU A | 74 | 38.196 | 9.519 | 47.730 | | | 1.00 | 40.21 | | A | C |
| ANISOU | 236 | CB | LEU A | 74 | 6860 | 3183 | 5234 | −1399 | 87 | | | −265 | A | C |
| ATOM | 237 | CG | LEU A | 74 | 37.577 | 9.108 | 46.405 | | | 1.00 | 48.99 | | A | C |
| ANISOU | 237 | CG | LEU A | 74 | 7952 | 4346 | 6317 | −1533 | 69 | | | −274 | A | C |
| ATOM | 238 | CD1 | LEU A | 74 | 36.079 | 9.229 | 46.463 | | | 1.00 | 50.42 | | A | C |
| ANISOU | 238 | CD1 | LEU A | 74 | 8075 | 4640 | 6441 | −1761 | 85 | | | −190 | A | C |
| ATOM | 239 | CD2 | LEU A | 74 | 38.126 | 9.994 | 45.296 | | | 1.00 | 60.72 | | A | C |
| ANISOU | 239 | CD2 | LEU A | 74 | 9341 | 5867 | 7862 | −1430 | 71 | | | −341 | A | C |
| ATOM | 240 | N | SER A | 75 | 40.437 | 9.276 | 50.151 | | | 1.00 | 44.10 | | A | N |
| ANISOU | 240 | N | SER A | 75 | 7473 | 3480 | 5801 | −1033 | 36 | | | −280 | A | N |
| ATOM | 241 | CA | SER A | 75 | 40.966 | 9.877 | 51.376 | | | 1.00 | 41.91 | | A | C |
| ANISOU | 241 | CA | SER A | 75 | 7217 | 3169 | 5539 | −899 | 12 | | | −275 | A | C |
| ATOM | 242 | C | SER A | 75 | 42.369 | 10.426 | 51.139 | | | 1.00 | 44.10 | | A | C |
| ANISOU | 242 | C | SER A | 75 | 7359 | 3431 | 5964 | −692 | −38 | | | −329 | A | C |
| ATOM | 243 | O | SER A | 75 | 42.684 | 11.551 | 51.550 | | | 1.00 | 37.81 | | A | O |
| ANISOU | 243 | O | SER A | 75 | 6487 | 2656 | 5224 | −581 | −66 | | | −346 | A | O |
| ATOM | 244 | CB | SER A | 75 | 40.943 | 8.876 | 52.533 | | | 1.00 | 46.11 | | A | C |
| ANISOU | 244 | CB | SER A | 75 | 7916 | 3618 | 5984 | −935 | 1 | | | −218 | A | C |
| ATOM | 245 | OG | SER A | 75 | 41.528 | 7.639 | 52.175 | | | 1.00 | 58.18 | | A | O |
| ANISOU | 245 | OG | SER A | 75 | 9479 | 5083 | 7542 | −933 | −27 | | | −225 | A | O |
| ATOM | 246 | N | ASN A | 76 | 43.189 | 9.688 | 50.377 | | | 1.00 | 47.35 | | A | N |
| ANISOU | 246 | N | ASN A | 76 | 7735 | 3808 | 6448 | −649 | −46 | | | −355 | A | N |
| ATOM | 247 | CA | ASN A | 76 | 44.579 | 10.085 | 50.134 | | | 1.00 | 41.60 | | A | C |
| ANISOU | 247 | CA | ASN A | 76 | 6872 | 3057 | 5876 | −482 | −71 | | | −387 | A | C |
| ATOM | 248 | C | ASN A | 76 | 44.683 | 11.368 | 49.298 | | | 1.00 | 33.03 | | A | C |
| ANISOU | 248 | C | ASN A | 76 | 5633 | 2037 | 4880 | −434 | −62 | | | −411 | A | C |
| ATOM | 249 | O | ASN A | 76 | 45.577 | 12.195 | 49.541 | | | 1.00 | 30.27 | | A | O |
| ANISOU | 249 | O | ASN A | 76 | 5160 | 1685 | 4656 | −312 | −78 | | | −415 | A | O |
| ATOM | 250 | CB | ASN A | 76 | 45.338 | 8.922 | 49.492 | | | 1.00 | 40.88 | | A | C |
| ANISOU | 250 | CB | ASN A | 76 | 6812 | 2897 | 5826 | −461 | −63 | | | −406 | A | C |
| ATOM | 251 | CG | ASN A | 76 | 45.993 | 8.026 | 50.530 | | | 1.00 | 55.01 | | A | C |
| ANISOU | 251 | CG | ASN A | 76 | 8677 | 4600 | 7623 | −414 | −93 | | | −383 | A | C |
| ATOM | 252 | OD1 | ASN A | 76 | 45.516 | 7.922 | 51.650 | | | 1.00 | 60.05 | | A | O |
| ANISOU | 252 | OD1 | ASN A | 76 | 9411 | 5229 | 8174 | −451 | −117 | | | −345 | A | O |
| ATOM | 253 | ND2 | ASN A | 76 | 47.101 | 7.390 | 50.164 | | | 1.00 | 68.33 | | A | N |
| ANISOU | 253 | ND2 | ASN A | 76 | 10337 | 6217 | 9410 | −331 | −90 | | | −403 | A | N |
| ATOM | 254 | N | ILE A | 77 | 43.795 | 11.546 | 48.305 | | | 1.00 | 30.11 | | A | N |
| ANISOU | 254 | N | ILE A | 77 | 5270 | 1723 | 4449 | −542 | −29 | | | −421 | A | N |
| ATOM | 255 | CA | ILE A | 77 | 43.771 | 12.767 | 47.488 | | | 1.00 | 26.61 | | A | C |
| ANISOU | 255 | CA | ILE A | 77 | 4700 | 1337 | 4076 | −514 | −25 | | | −435 | A | C |
| ATOM | 256 | C | ILE A | 77 | 43.168 | 13.923 | 48.267 | | | 1.00 | 28.36 | | A | C |
| ANISOU | 256 | C | ILE A | 77 | 4885 | 1600 | 4291 | −505 | −44 | | | −424 | A | C |
| ATOM | 257 | O | ILE A | 77 | 43.596 | 15.095 | 48.173 | | | 1.00 | 26.44 | | A | C |
| ANISOU | 257 | O | ILE A | 77 | 4517 | 1371 | 4157 | −411 | −67 | | | −432 | A | O |
| ATOM | 258 | CB | ILE A | 77 | 42.975 | 12.504 | 46.201 | | | 1.00 | 35.47 | | A | O |
| ANISOU | 258 | CB | ILE A | 77 | 5862 | 2503 | 5113 | −648 | 18 | | | −446 | A | C |
| ATOM | 259 | CG1 | ILE A | 77 | 43.558 | 11.298 | 45.464 | | | 1.00 | 27.74 | | A | C |
| ANISOU | 259 | CG1 | ILE A | 77 | 4966 | 1461 | 4114 | −642 | 35 | | | −472 | A | C |
| ATOM | 260 | CG2 | ILE A | 77 | 42.886 | 13.769 | 45.354 | | | 1.00 | 26.02 | | A | C |
| ANISOU | 260 | CG2 | ILE A | 77 | 4549 | 1362 | 3974 | −634 | 23 | | | −451 | A | C |
| ATOM | 261 | CD1 | ILE A | 77 | 42.601 | 10.698 | 44.478 | | | 1.00 | 28.51 | | A | C |
| ANISOU | 261 | CD1 | ILE A | 77 | 5159 | 1588 | 4087 | −801 | 65 | | | −487 | A | C |
| ATOM | 262 | N | SER A | 78 | 42.124 | 13.603 | 49.009 | | | 1.00 | 35.73 | | A | N |
| ANISOU | 262 | N | SER A | 78 | 5939 | 2544 | 5093 | −613 | −26 | | | −404 | A | N |
| ATOM | 263 | CA | SER A | 78 | 41.533 | 14.530 | 49.955 | | | 1.00 | 40.34 | | A | C |
| ANISOU | 263 | CA | SER A | 78 | 6551 | 3139 | 5638 | −595 | −37 | | | −400 | A | C |
| ATOM | 264 | C | SER A | 78 | 42.587 | 15.111 | 50.903 | | | 1.00 | 35.58 | | A | C |
| ANISOU | 264 | C | SER A | 78 | 5904 | 2493 | 5123 | −404 | −110 | | | −413 | A | C |
| ATOM | 265 | O | SER A | 78 | 42.535 | 16.297 | 51.274 | | | 1.00 | 27.11 | | A | O |
| ANISOU | 265 | O | SER A | 78 | 4783 | 1433 | 4084 | −316 | −151 | | | −431 | A | O |
| ATOM | 266 | CB | SER A | 78 | 40.454 | 13.779 | 50.726 | | | 1.00 | 47.62 | | A | C |
| ANISOU | 266 | CB | SER A | 78 | 7645 | 4050 | 6400 | −748 | 17 | | | −359 | A | C |
| ATOM | 267 | OG | SER A | 78 | 39.876 | 14.626 | 51.695 | | | 1.00 | 66.19 | | A | O |
| ANISOU | 267 | OG | SER A | 78 | 10100 | 6354 | 8698 | −661 | −5 | | | −356 | A | O |
| ATOM | 268 | N | ASP A | 79 | 43.571 | 14.291 | 51.285 | | | 1.00 | 31.96 | | A | N |
| ANISOU | 268 | N | ASP A | 79 | 5457 | 1985 | 4703 | −344 | −128 | | | −404 | A | N |
| ATOM | 269 | CA | ASP A | 79 | 44.591 | 14.738 | 52.231 | | | 1.00 | 37.06 | | A | C |
| ANISOU | 269 | CA | ASP A | 79 | 6067 | 2602 | 5413 | −200 | −181 | | | −403 | A | C |
| ATOM | 270 | C | ASP A | 79 | 45.445 | 15.866 | 51.663 | | | 1.00 | 44.41 | | A | C |
| ANISOU | 270 | C | ASP A | 79 | 6832 | 3565 | 6475 | −109 | −173 | | | −405 | A | C |
| ATOM | 271 | O | ASP A | 79 | 45.833 | 16.781 | 52.403 | | | 1.00 | 51.82 | | A | O |
| ANISOU | 271 | O | ASP A | 79 | 7757 | 4516 | 7418 | −14 | −236 | | | −397 | A | O |
| ATOM | 272 | CB | ASP A | 79 | 45.467 | 13.559 | 52.668 | | | 1.00 | 42.08 | | A | C |
| ANISOU | 272 | CB | ASP A | 79 | 6753 | 3175 | 6060 | −180 | −187 | | | −382 | A | C |
| ATOM | 273 | CG | ASP A | 79 | 44.736 | 12.602 | 53.624 | | | 1.00 | 61.16 | | A | C |
| ANISOU | 273 | CG | ASP A | 79 | 9378 | 5547 | 8314 | −253 | −201 | | | −356 | A | C |
| ATOM | 274 | OD1 | ASP A | 79 | 43.692 | 12.988 | 54.198 | | | 1.00 | 65.98 | | A | O |
| ANISOU | 274 | OD1 | ASP A | 79 | 10106 | 6167 | 8797 | −298 | −198 | | | −349 | A | O |
| ATOM | 275 | OD2 | ASP A | 79 | 45.208 | 11.465 | 53.841 | | | 1.00 | 65.70 | | A | O1− |
| ANISOU | 275 | OD2 | ASP A | 79 | 10017 | 6064 | 8880 | −268 | −204 | | | −336 | A | O1− |
| ATOM | 276 | N | ILE A | 80 | 45.744 | 15.842 | 50.360 | | | 1.00 | 39.50 | | A | N |
| ANISOU | 276 | N | ILE A | 80 | 6122 | 2958 | 5930 | −139 | −103 | | | −405 | A | N |
| ATOM | 277 | CA | ILE A | 80 | 46.558 | 16.927 | 49.814 | | | 1.00 | 34.04 | | A | C |
| ANISOU | 277 | CA | ILE A | 80 | 5358 | 2296 | 5280 | −63 | −87 | | | −372 | A | C |
| ATOM | 278 | C | ILE A | 80 | 45.721 | 18.093 | 49.307 | | | 1.00 | 28.37 | | A | C |
| ANISOU | 278 | C | ILE A | 80 | 4593 | 1640 | 4548 | −81 | −124 | | | −379 | A | C |
| ATOM | 279 | O | ILE A | 80 | 46.294 | 19.135 | 48.960 | | | 1.00 | 23.66 | | A | O |
| ANISOU | 279 | O | ILE A | 80 | 3910 | 1082 | 3996 | −9 | −189 | | | −342 | A | O |
| ATOM | 280 | CB | ILE A | 80 | 47.508 | 16.426 | 48.708 | | | 1.00 | 35.19 | | A | C |
| ANISOU | 280 | CB | ILE A | 80 | 5498 | 2411 | 5460 | −54 | −3 | | | −348 | A | C |
| ATOM | 281 | CG1 | ILE A | 80 | 46.761 | 16.162 | 47.409 | | | 1.00 | 41.25 | | A | C |
| ANISOU | 281 | CG1 | ILE A | 80 | 6235 | 3218 | 6221 | −148 | 51 | | | −375 | A | C |
| ATOM | 282 | CG2 | ILE A | 80 | 48.172 | 15.132 | 49.138 | | | 1.00 | 33.60 | | A | C |
| ANISOU | 282 | CG2 | ILE A | 80 | 5319 | 2150 | 5299 | −42 | 25 | | | −351 | A | C |
| ATOM | 283 | CD1 | ILE A | 80 | 47.638 | 15.507 | 46.337 | | | 1.00 | 26.52 | | A | C |
| ANISOU | 283 | CD1 | ILE A | 80 | 4384 | 1326 | 4366 | −125 | 124 | | | −360 | A | C |
| ATOM | 284 | N | PHE A | 81 | 44.386 | 17.970 | 49.258 | | | 1.00 | 33.24 | | A | N |
| ANISOU | 284 | N | PHE A | 81 | 5235 | 2267 | 5128 | −175 | −120 | | | −418 | A | N |
| ATOM | 285 | CA | PHE A | 81 | 43.590 | 19.120 | 48.829 | | | 1.00 | 32.09 | | A | C |
| ANISOU | 285 | CA | PHE A | 81 | 5033 | 2171 | 4988 | −186 | −167 | | | −423 | A | C |
| ATOM | 286 | C | PHE A | 81 | 42.785 | 19.835 | 49.907 | | | 1.00 | 46.01 | | A | C |
| ANISOU | 286 | C | PHE A | 81 | 6827 | 3935 | 6719 | −132 | −250 | | | −452 | A | C |
| ATOM | 287 | O | PHE A | 81 | 42.121 | 20.821 | 49.574 | | | 1.00 | 52.42 | | A | O |
| ANISOU | 287 | O | PHE A | 81 | 7557 | 4782 | 7579 | −120 | −302 | | | −466 | A | O |
| ATOM | 288 | CB | PHE A | 81 | 42.640 | 18.727 | 47.706 | | | 1.00 | 26.08 | | A | C |
| ANISOU | 288 | CB | PHE A | 81 | 4269 | 1427 | 4213 | −342 | −118 | | | −437 | A | C |
| ATOM | 289 | CG | PHE A | 81 | 43.339 | 18.484 | 46.396 | | | 1.00 | 23.48 | | A | C |
| ANISOU | 289 | CG | PHE A | 81 | 3915 | 1100 | 3906 | −364 | −60 | | | −416 | A | C |
| ATOM | 290 | CD1 | PHE A | 81 | 43.894 | 19.545 | 45.675 | | | 1.00 | 22.98 | | A | C |
| ANISOU | 290 | CD1 | PHE A | 81 | 3724 | 1081 | 3926 | −287 | −119 | | | −374 | A | C |
| ATOM | 291 | CD2 | PHE A | 81 | 43.419 | 17.209 | 45.872 | | | 1.00 | 23.98 | | A | C |
| ANISOU | 291 | CD2 | PHE A | 81 | 3983 | 1138 | 3990 | −421 | −63 | | | −430 | A | C |
| ATOM | 292 | CE1 | PHE A | 81 | 44.524 | 19.333 | 44.513 | | | 1.00 | 23.01 | | A | C |
| ANISOU | 292 | CE1 | PHE A | 81 | 3707 | 1077 | 3960 | −292 | −84 | | | −349 | A | C |
| ATOM | 293 | CE2 | PHE A | 81 | 44.049 | 16.997 | 44.682 | | | 1.00 | 30.82 | | A | C |
| ANISOU | 293 | CE2 | PHE A | 81 | 4844 | 1996 | 4872 | −409 | −72 | | | −413 | A | C |
| ATOM | 294 | CZ | PHE A | 81 | 44.614 | 18.061 | 44.006 | | | 1.00 | 25.71 | | A | C |
| ANISOU | 294 | CZ | PHE A | 81 | 4198 | 1361 | 4210 | −360 | −45 | | | −370 | A | C |
| ATOM | 295 | N | ASN A | 82 | 42.785 | 19.367 | 51.161 | | | 1.00 | 44.06 | | A | N |
| ANISOU | 295 | N | ASN A | 82 | 6698 | 3646 | 6395 | −90 | −271 | | | −466 | A | N |
| ATOM | 296 | CA | ASN A | 82 | 42.104 | 20.067 | 52.266 | | | 1.00 | 43.67 | | A | C |
| ANISOU | 296 | CA | ASN A | 82 | 6735 | 3585 | 6274 | 1 | −345 | | | −502 | A | C |
| ATOM | 297 | C | ASN A | 82 | 40.588 | 20.057 | 52.119 | | | 1.00 | 38.27 | | A | C |
| ANISOU | 297 | C | ASN A | 82 | 6035 | 3043 | 5463 | −95 | −246 | | | −492 | A | C |
| ATOM | 298 | O | ASN A | 82 | 39.937 | 21.107 | 52.174 | | | 1.00 | 45.53 | | A | O |
| ANISOU | 298 | O | ASN A | 82 | 6839 | 4082 | 6377 | −22 | −251 | | | −503 | A | O |
| ATOM | 299 | CB | ASN A | 82 | 42.558 | 21.522 | 52.413 | | | 1.00 | 52.07 | | A | C |
| ANISOU | 299 | CB | ASN A | 82 | 7669 | 4691 | 7424 | 146 | −442 | | | −510 | A | C |
| ATOM | 300 | CG | ASN A | 82 | 43.950 | 21.665 | 52.949 | | | 1.00 | 50.89 | | A | C |
| ANISOU | 300 | CG | ASN A | 82 | 7483 | 4544 | 7308 | 223 | −500 | | | −472 | A | C |
| ATOM | 301 | OD1 | ASN A | 82 | 44.602 | 20.685 | 53.340 | | | 1.00 | 53.99 | | A | O |
| ANISOU | 301 | OD1 | ASN A | 82 | 7939 | 4899 | 7675 | 199 | −470 | | | −451 | A | O |
| ATOM | 302 | ND2 | ASN A | 82 | 44.415 | 22.912 | 52.990 | | | 1.00 | 42.82 | | A | N |
| ANISOU | 302 | ND2 | ASN A | 82 | 6358 | 3561 | 6351 | 311 | −602 | | | −461 | A | N |
| ATOM | 303 | N | LEU A | 83 | 40.021 | 18.875 | 51.909 | | | 1.00 | 27.33 | | A | N |
| ANISOU | 303 | N | LEU A | 83 | 4706 | 1705 | 3973 | −261 | −134 | | | −450 | A | N |
| ATOM | 304 | CA | LEU A | 83 | 38.567 | 18.731 | 51.894 | | | 1.00 | 28.54 | | A | C |
| ANISOU | 304 | CA | LEU A | 83 | 4783 | 2064 | 3996 | −372 | −12 | | | −399 | A | C |
| ATOM | 305 | C | LEU A | 83 | 38.237 | 17.257 | 52.101 | | | 1.00 | 30.85 | | A | C |
| ANISOU | 305 | C | LEU A | 83 | 5210 | 2335 | 4178 | −539 | 67 | | | −345 | A | C |
| ATOM | 306 | O | LEU A | 83 | 39.120 | 16.402 | 52.113 | | | 1.00 | 31.30 | | A | O |
| ANISOU | 306 | O | LEU A | 83 | 5412 | 2216 | 4263 | −562 | 28 | | | −360 | A | O |
| ATOM | 307 | CB | LEU A | 83 | 37.942 | 19.294 | 50.601 | | | 1.00 | 41.10 | | A | C |
| ANISOU | 307 | CB | LEU A | 83 | 6172 | 3793 | 5653 | −456 | 17 | | | −381 | A | C |
| ATOM | 308 | CG | LEU A | 83 | 38.271 | 18.721 | 49.210 | | | 1.00 | 33.52 | | A | C |
| ANISOU | 308 | CG | LEU A | 83 | 5162 | 2756 | 4820 | −514 | −21 | | | −396 | A | C |
| ATOM | 309 | CD1 | LEU A | 83 | 37.498 | 17.431 | 48.874 | | | 1.00 | 29.44 | | A | C |
| ANISOU | 309 | CD1 | LEU A | 83 | 4772 | 2154 | 4261 | −675 | 24 | | | −384 | A | C |
| ATOM | 310 | CD2 | LEU A | 83 | 38.041 | 19.792 | 48.152 | | | 1.00 | 26.22 | | A | C |
| ANISOU | 310 | CD2 | LEU A | 83 | 4034 | 1992 | 3938 | −554 | −8 | | | −372 | A | C |
| ATOM | 311 | N | SER A | 84 | 36.962 | 16.965 | 52.283 | | | 1.00 | 31.49 | | A | N |
| ANISOU | 311 | N | SER A | 84 | 5234 | 2589 | 4143 | −653 | 173 | | | −273 | A | N |
| ATOM | 312 | CA | SER A | 84 | 36.576 | 15.604 | 52.613 | | | 1.00 | 32.35 | | A | C |
| ANISOU | 312 | CA | SER A | 84 | 5468 | 2675 | 4149 | −819 | 237 | | | −201 | A | C |
| ATOM | 313 | C | SER A | 84 | 36.619 | 14.713 | 51.373 | | | 1.00 | 29.45 | | A | C |
| ANISOU | 313 | C | SER A | 84 | 5112 | 2243 | 3833 | −1015 | 229 | | | −194 | A | C |
| ATOM | 314 | O | SER A | 84 | 36.458 | 15.192 | 50.252 | | | 1.00 | 40.82 | | A | O |
| ANISOU | 314 | O | SER A | 84 | 6418 | 3744 | 5346 | −1055 | 216 | | | −216 | A | O |
| ATOM | 315 | CB | SER A | 84 | 35.177 | 15.616 | 53.224 | | | 1.00 | 43.32 | | A | C |
| ANISOU | 315 | CB | SER A | 84 | 6768 | 4282 | 5410 | −880 | 356 | | | −101 | A | C |
| ATOM | 316 | OG | SER A | 84 | 34.304 | 16.553 | 52.566 | | | 1.00 | 49.11 | | A | O |
| ANISOU | 316 | OG | SER A | 84 | 7269 | 5208 | 6184 | −881 | 391 | | | −86 | A | O |
| ATOM | 317 | N | PRO A | 85 | 36.809 | 13.403 | 51.535 | | | 1.00 | 30.18 | | A | N |
| ANISOU | 317 | N | PRO A | 85 | 5378 | 2209 | 3881 | −1139 | 231 | | | −165 | A | N |
| ATOM | 318 | CA | PRO A | 85 | 36.553 | 12.512 | 50.394 | | | 1.00 | 30.48 | | A | C |
| ANISOU | 318 | CA | PRO A | 85 | 5440 | 2206 | 3936 | −1343 | 226 | | | −153 | A | C |
| ATOM | 319 | C | PRO A | 85 | 35.150 | 12.662 | 49.838 | | | 1.00 | 50.41 | | A | C |
| ANISOU | 319 | C | PRO A | 85 | 7789 | 4939 | 6425 | −1513 | 276 | | | −74 | A | C |
| ATOM | 320 | O | PRO A | 85 | 34.921 | 12.361 | 48.662 | | | 1.00 | 58.33 | | A | O |
| ANISOU | 320 | O | PRO A | 85 | 8766 | 5939 | 7458 | −1651 | 246 | | | −86 | A | O |
| ATOM | 321 | CB | PRO A | 85 | 36.763 | 11.104 | 50.969 | | | 1.00 | 31.60 | | A | C |
| ANISOU | 321 | CB | PRO A | 85 | 5804 | 2187 | 4017 | −1446 | 219 | | | −114 | A | C |
| ATOM | 322 | CG | PRO A | 85 | 37.378 | 11.264 | 52.291 | | | 1.00 | 31.62 | | A | C |
| ANISOU | 322 | CG | PRO A | 85 | 5906 | 2123 | 3986 | −1278 | 209 | | | −116 | A | C |
| ATOM | 323 | CD | PRO A | 85 | 37.086 | 12.657 | 52.774 | | | 1.00 | 34.36 | | A | C |
| ANISOU | 323 | CD | PRO A | 85 | 6093 | 2637 | 4325 | −1113 | 235 | | | −128 | A | C |
| ATOM | 324 | N | LEU A | 86 | 34.187 | 13.100 | 50.652 | | | 1.00 | 51.28 | | A | N |
| ANISOU | 324 | N | LEU A | 86 | 7779 | 5234 | 6470 | −1502 | 351 | | | 12 | A | N |
| ATOM | 325 | CA | LEU A | 86 | 32.821 | 13.248 | 50.160 | | | 1.00 | 54.62 | | A | C |
| ANISOU | 325 | CA | LEU A | 86 | 8006 | 5867 | 6879 | −1660 | 399 | | | 108 | A | C |
| ATOM | 326 | C | LEU A | 86 | 32.768 | 14.251 | 49.024 | | | 1.00 | 44.39 | | A | C |
| ANISOU | 326 | C | LEU A | 86 | 6543 | 4653 | 5672 | −1619 | 358 | | | 49 | A | C |
| ATOM | 327 | O | LEU A | 86 | 32.116 | 14.015 | 48.004 | | | 1.00 | 39.57 | | A | O |
| ANISOU | 327 | O | LEU A | 86 | 5849 | 4105 | 5081 | −1795 | 331 | | | 84 | A | O |
| ATOM | 328 | CB | LEU A | 86 | 31.882 | 13.693 | 51.274 | | | 1.00 | 59.57 | | A | C |
| ANISOU | 328 | CB | LEU A | 86 | 8513 | 6694 | 7426 | −1601 | 509 | | | 210 | A | C |
| ATOM | 329 | CG | LEU A | 86 | 30.438 | 13.975 | 50.857 | | | 1.00 | 60.40 | | A | C |
| ANISOU | 329 | CG | LEU A | 86 | 8370 | 7043 | 7538 | −1736 | 569 | | | 328 | A | C |
| ATOM | 330 | CD1 | LEU A | 86 | 29.766 | 12.699 | 50.354 | | | 1.00 | 48.56 | | A | C |
| ANISOU | 330 | CD1 | LEU A | 86 | 6895 | 5522 | 6035 | −2043 | 546 | | | 439 | A | C |
| ATOM | 331 | CD2 | LEU A | 86 | 29.698 | 14.543 | 52.037 | | | 1.00 | 77.58 | | A | C |
| ANISOU | 331 | CD2 | LEU A | 86 | 10434 | 9413 | 9630 | −1607 | 700 | | | 414 | A | C |
| ATOM | 332 | N | ARG A | 87 | 33.420 | 15.397 | 49.197 | | | 1.00 | 30.63 | | A | N |
| ANISOU | 332 | N | ARG A | 87 | 4749 | 2907 | 3980 | −1392 | 340 | | | −32 | A | N |
| ATOM | 333 | CA | ARG A | 87 | 33.286 | 16.438 | 48.198 | | | 1.00 | 29.78 | | A | C |
| ANISOU | 333 | CA | ARG A | 87 | 4467 | 2891 | 3956 | −1349 | 304 | | | −65 | A | C |
| ATOM | 334 | C | ARG A | 87 | 33.850 | 15.984 | 46.865 | | | 1.00 | 45.48 | | A | C |
| ANISOU | 334 | C | ARG A | 87 | 6521 | 4765 | 5994 | −1452 | 236 | | | −116 | A | C |
| ATOM | 335 | O | ARG A | 87 | 33.266 | 16.254 | 45.805 | | | 1.00 | 49.67 | | A | O |
| ANISOU | 335 | O | ARG A | 87 | 6929 | 5396 | 6547 | −1554 | 213 | | | −95 | A | O |
| ATOM | 336 | CB | ARG A | 87 | 33.975 | 17.700 | 48.667 | | | 1.00 | 28.72 | | A | C |
| ANISOU | 336 | CB | ARG A | 87 | 4291 | 2739 | 3882 | −1093 | 274 | | | −138 | A | C |
| ATOM | 337 | CG | ARG A | 87 | 33.175 | 18.461 | 49.634 | | | 1.00 | 29.48 | | A | C |
| ANISOU | 337 | CG | ARG A | 87 | 4278 | 2997 | 3927 | −976 | 338 | | | −98 | A | C |
| ATOM | 338 | CD | ARG A | 87 | 33.829 | 19.788 | 49.921 | | | 1.00 | 41.62 | | A | C |
| ANISOU | 338 | CD | ARG A | 87 | 5781 | 4497 | 5535 | −730 | 273 | | | −182 | A | C |
| ATOM | 339 | NE | ARG A | 87 | 33.793 | 20.718 | 48.796 | | | 1.00 | 43.59 | | A | N |
| ANISOU | 339 | NE | ARG A | 87 | 5867 | 4797 | 5899 | −717 | 215 | | | −199 | A | N |
| ATOM | 340 | CZ | ARG A | 87 | 34.089 | 22.012 | 48.925 | | | 1.00 | 48.77 | | A | C |
| ANISOU | 340 | CZ | ARG A | 87 | 6447 | 5450 | 6633 | −525 | 152 | | | −249 | A | C |
| ATOM | 341 | NH1 | ARG A | 87 | 34.031 | 22.812 | 47.864 | | | 1.00 | 41.79 | | A | N1+ |
| ANISOU | 341 | NH1 | ARG A | 87 | 5415 | 4610 | 5854 | −530 | 97 | | | −247 | A | N1+ |
| ATOM | 342 | NH2 | ARG A | 87 | 34.435 | 22.507 | 50.132 | | | 1.00 | 48.53 | | A | N |
| ANISOU | 342 | NH2 | ARG A | 87 | 6504 | 5365 | 6570 | −328 | 131 | | | −299 | A | N |
| ATOM | 343 | N | ILE A | 88 | 34.978 | 15.273 | 46.903 | | | 1.00 | 42.69 | | A | N |
| ANISOU | 343 | N | ILE A | 88 | 6367 | 4204 | 5649 | −1420 | 206 | | | −181 | A | N |
| ATOM | 344 | CA | ILE A | 88 | 35.557 | 14.744 | 45.678 | | | 1.00 | 37.36 | | A | C |
| ANISOU | 344 | CA | ILE A | 88 | 5782 | 3415 | 4998 | −1494 | 163 | | | −234 | A | C |
| ATOM | 345 | C | ILE A | 88 | 34.618 | 13.710 | 45.067 | | | 1.00 | 41.23 | | A | C |
| ANISOU | 345 | C | ILE A | 88 | 6318 | 3932 | 5414 | −1748 | 153 | | | −183 | A | C |
| ATOM | 346 | O | ILE A | 88 | 34.350 | 13.737 | 43.862 | | | 1.00 | 46.70 | | A | O |
| ANISOU | 346 | O | ILE A | 88 | 6977 | 4662 | 6106 | −1845 | 115 | | | −196 | A | O |
| ATOM | 347 | CB | ILE A | 88 | 36.963 | 14.181 | 45.954 | | | 1.00 | 32.76 | | A | C |
| ANISOU | 347 | CB | ILE A | 88 | 5394 | 2605 | 4448 | −1381 | 144 | | | −306 | A | C |
| ATOM | 348 | CG1 | ILE A | 88 | 37.858 | 15.293 | 46.479 | | | 1.00 | 26.83 | | A | C |
| ANISOU | 348 | CG1 | ILE A | 88 | 4573 | 1828 | 3792 | −1147 | 122 | | | −343 | A | C |
| ATOM | 349 | CG2 | ILE A | 88 | 37.573 | 13.616 | 44.684 | | | 1.00 | 37.72 | | A | C |
| ANISOU | 349 | CG2 | ILE A | 88 | 6073 | 3174 | 5085 | −1379 | 116 | | | −352 | A | C |
| ATOM | 350 | CD1 | ILE A | 88 | 39.257 | 14.821 | 46.815 | | | 1.00 | 31.57 | | A | C |
| ANISOU | 350 | CD1 | ILE A | 88 | 5304 | 2242 | 4450 | −1008 | 88 | | | −389 | A | C |
| ATOM | 351 | N | ALA A | 89 | 34.044 | 12.829 | 45.897 | | | 1.00 | 37.95 | | A | N |
| ANISOU | 351 | N | ALA A | 89 | 5978 | 3506 | 4936 | −1868 | 176 | | | −113 | A | N |
| ATOM | 352 | CA | ALA A | 89 | 33.108 | 11.828 | 45.379 | | | 1.00 | 32.60 | | A | C |
| ANISOU | 352 | CA | ALA A | 89 | 5329 | 2850 | 4206 | −2115 | 141 | | | −46 | A | C |
| ATOM | 353 | C | ALA A | 89 | 31.901 | 12.475 | 44.709 | | | 1.00 | 37.73 | | A | C |
| ANISOU | 353 | C | ALA A | 89 | 5758 | 3710 | 4869 | −2245 | 130 | | | 27 | A | C |
| ATOM | 354 | O | ALA A | 89 | 31.416 | 11.985 | 43.684 | | | 1.00 | 46.97 | | A | O |
| ANISOU | 354 | O | ALA A | 89 | 6916 | 4908 | 6022 | −2352 | 53 | | | 36 | A | O |
| ATOM | 355 | CB | ALA A | 89 | 32.644 | 10.881 | 46.486 | | | 1.00 | 33.98 | | A | C |
| ANISOU | 355 | CB | ALA A | 89 | 5586 | 2998 | 4326 | −2219 | 172 | | | 50 | A | C |
| ATOM | 356 | N | LYS A | 90 | 31.386 | 13.565 | 45.273 | | | 1.00 | 36.57 | | A | N |
| ANISOU | 356 | N | LYS A | 90 | 5394 | 3750 | 4750 | −2132 | 191 | | | 78 | A | N |
| ATOM | 357 | CA | LYS A | 90 | 30.193 | 14.172 | 44.695 | | | 1.00 | 37.29 | | A | C |
| ANISOU | 357 | CA | LYS A | 90 | 5249 | 4055 | 4866 | −2231 | 179 | | | 161 | A | C |
| ATOM | 358 | C | LYS A | 90 | 30.526 | 14.975 | 43.450 | | | 1.00 | 44.55 | | A | C |
| ANISOU | 358 | C | LYS A | 90 | 6106 | 4991 | 5832 | −2175 | 115 | | | 87 | A | C |
| ATOM | 359 | O | LYS A | 90 | 29.705 | 15.045 | 42.523 | | | 1.00 | 53.45 | | A | O |
| ANISOU | 359 | O | LYS A | 90 | 7124 | 6220 | 6964 | −2326 | 53 | | | 137 | A | O |
| ATOM | 360 | CB | LYS A | 90 | 29.468 | 15.048 | 45.721 | | | 1.00 | 33.76 | | A | C |
| ANISOU | 360 | CB | LYS A | 90 | 4592 | 3806 | 4428 | −2113 | 276 | | | 244 | A | C |
| ATOM | 361 | CG | LYS A | 90 | 28.806 | 14.247 | 46.864 | | | 1.00 | 49.80 | | A | C |
| ANISOU | 361 | CG | LYS A | 90 | 6640 | 5884 | 6398 | −2207 | 358 | | | 366 | A | C |
| ATOM | 362 | CD | LYS A | 90 | 27.687 | 15.019 | 47.584 | | | 1.00 | 64.87 | | A | C |
| ANISOU | 362 | CD | LYS A | 90 | 8294 | 8049 | 8305 | −2143 | 466 | | | 485 | A | C |
| ATOM | 363 | CE | LYS A | 90 | 28.188 | 16.259 | 48.342 | | | 1.00 | 73.33 | | A | C |
| ANISOU | 363 | CE | LYS A | 90 | 9327 | 9163 | 9373 | −1829 | 529 | | | 404 | A | C |
| ATOM | 364 | NZ | LYS A | 90 | 27.077 | 16.921 | 49.102 | | | 1.00 | 76.22 | | A | N1+ |
| ANISOU | 364 | NZ | LYS A | 90 | 9470 | 9774 | 9716 | −1745 | 651 | | | 515 | A | N1+ |
| ATOM | 365 | N | ALA A | 91 | 31.730 | 15.550 | 43.385 | | | 1.00 | 37.90 | | A | N |
| ANISOU | 365 | N | ALA A | 91 | 5332 | 4044 | 5025 | −1970 | 118 | | | −19 | A | N |
| ATOM | 366 | CA | ALA A | 91 | 32.117 | 16.278 | 42.182 | | | 1.00 | 29.97 | | A | C |
| ANISOU | 366 | CA | ALA A | 91 | 4275 | 3051 | 4062 | −1918 | 66 | | | −71 | A | C |
| ATOM | 367 | C | ALA A | 91 | 32.497 | 15.337 | 41.037 | | | 1.00 | 38.82 | | A | C |
| ANISOU | 367 | C | ALA A | 91 | 5584 | 4042 | 5125 | −2051 | 6 | | | −125 | A | C |
| ATOM | 368 | O | ALA A | 91 | 32.286 | 15.662 | 39.856 | | | 1.00 | 35.13 | | A | O |
| ANISOU | 368 | O | ALA A | 91 | 5070 | 3631 | 4647 | −2106 | −49 | | | −130 | A | O |
| ATOM | 369 | CB | ALA A | 91 | 33.255 | 17.233 | 42.501 | | | 1.00 | 28.41 | | A | C |
| ANISOU | 369 | CB | ALA A | 91 | 4067 | 2790 | 3939 | −1666 | 86 | | | −140 | A | C |
| ATOM | 370 | N | SER A | 92 | 33.034 | 14.165 | 41.363 | | | 1.00 | 34.42 | | A | N |
| ANISOU | 370 | N | SER A | 92 | 5243 | 3316 | 4520 | −2076 | 11 | | | −165 | A | N |
| ATOM | 371 | CA | SER A | 92 | 33.494 | 13.192 | 40.378 | | | 1.00 | 31.48 | | A | C |
| ANISOU | 371 | CA | SER A | 92 | 5041 | 2839 | 4082 | −2075 | −39 | | | −226 | A | C |
| ATOM | 372 | C | SER A | 92 | 32.397 | 12.252 | 39.890 | | | 1.00 | 38.94 | | A | C |
| ANISOU | 372 | C | SER A | 92 | 6008 | 3830 | 4955 | −2264 | −116 | | | −172 | A | C |
| ATOM | 373 | O | SER A | 92 | 32.632 | 11.476 | 38.957 | | | 1.00 | 45.25 | | A | O |
| ANISOU | 373 | O | SER A | 92 | 6961 | 4548 | 5685 | −2287 | −172 | | | −227 | A | O |
| ATOM | 374 | CB | SER A | 92 | 34.639 | 12.369 | 40.978 | | | 1.00 | 40.72 | | A | C |
| ANISOU | 374 | CB | SER A | 92 | 6394 | 3829 | 5248 | −1938 | −6 | | | −288 | A | C |
| ATOM | 375 | OG | SER A | 92 | 35.686 | 13.225 | 41.440 | | | 1.00 | 35.93 | | A | C |
| ANISOU | 375 | OG | SER A | 92 | 5758 | 3170 | 4723 | −1751 | 46 | | | −329 | A | O |
| ATOM | 376 | N | ASN A | 93 | 31.213 | 12.298 | 40.505 | | | 1.00 | 41.81 | | A | N |
| ANISOU | 376 | N | ASN A | 93 | 6226 | 4321 | 5339 | −2399 | −120 | | | −61 | A | N |
| ATOM | 377 | CA | ASN A | 93 | 30.100 | 11.386 | 40.212 | | | 1.00 | 37.39 | | A | C |
| ANISOU | 377 | CA | ASN A | 93 | 5663 | 3804 | 4741 | −2585 | −202 | | | 16 | A | C |
| ATOM | 378 | C | ASN A | 93 | 30.435 | 9.944 | 40.609 | | | 1.00 | 42.72 | | A | C |
| ANISOU | 378 | C | ASN A | 93 | 6550 | 4312 | 5368 | −2610 | −223 | | | −3 | A | C |
| ATOM | 379 | O | ASN A | 93 | 30.073 | 8.983 | 39.926 | | | 1.00 | 41.63 | | A | O |
| ANISOU | 379 | O | ASN A | 93 | 6522 | 4117 | 5180 | −2717 | −319 | | | −6 | A | O |
| ATOM | 380 | CB | ASN A | 93 | 29.661 | 11.484 | 38.748 | | | 1.00 | 36.98 | | A | C |
| ANISOU | 380 | CB | ASN A | 93 | 5600 | 3802 | 4649 | −2659 | −305 | | | −1 | A | C |
| ATOM | 381 | CG | ASN A | 93 | 28.337 | 10.795 | 38.481 | | | 1.00 | 48.90 | | A | C |
| ANISOU | 381 | CG | ASN A | 93 | 7052 | 5382 | 6147 | −2857 | −406 | | | 100 | A | C |
| ATOM | 382 | OD1 | ASN A | 93 | 27.369 | 10.906 | 39.244 | | | 1.00 | 49.43 | | A | O |
| ANISOU | 382 | OD1 | ASN A | 93 | 6938 | 5571 | 6273 | −2944 | −383 | | | 225 | A | O |
| ATOM | 383 | ND2 | ASN A | 93 | 28.296 | 10.061 | 37.379 | | | 1.00 | 60.69 | | A | N |
| ANISOU | 383 | ND2 | ASN A | 93 | 8705 | 6792 | 7561 | −2930 | −519 | | | 50 | A | N |
| ATOM | 384 | N | ILE A | 94 | 31.062 | 9.787 | 41.769 | | | 1.00 | 53.62 | | A | N |
| ANISOU | 384 | N | ILE A | 94 | 7995 | 5611 | 6766 | −2513 | −143 | | | −13 | A | N |
| ATOM | 385 | CA | ILE A | 94 | 31.422 | 8.460 | 42.250 | | | 1.00 | 60.33 | | A | C |
| ANISOU | 385 | CA | ILE A | 94 | 9014 | 6322 | 7586 | −2535 | −154 | | | −4 | A | C |
| ATOM | 386 | C | ILE A | 94 | 30.409 | 7.959 | 43.276 | | | 1.00 | 62.25 | | A | C |
| ANISOU | 386 | C | ILE A | 94 | 9144 | 6660 | 7847 | −2676 | −119 | | | 145 | A | C |
| ATOM | 387 | O | ILE A | 94 | 30.173 | 8.600 | 44.297 | | | 1.00 | 59.20 | | A | O |
| ANISOU | 387 | O | ILE A | 94 | 8623 | 6379 | 7489 | −2650 | −26 | | | 202 | A | O |
| ATOM | 388 | CB | ILE A | 94 | 32.823 | 8.459 | 42.881 | | | 1.00 | 51.19 | | A | C |
| ANISOU | 388 | CB | ILE A | 94 | 7994 | 5018 | 6437 | −2327 | −95 | | | −98 | A | C |
| ATOM | 389 | CG1 | ILE A | 94 | 33.858 | 8.929 | 41.865 | | | 1.00 | 47.82 | | A | C |
| ANISOU | 389 | CG1 | ILE A | 94 | 7657 | 4515 | 5997 | −2192 | −114 | | | −220 | A | C |
| ATOM | 390 | CG2 | ILE A | 94 | 33.176 | 7.075 | 43.391 | | | 1.00 | 51.23 | | A | C |
| ANISOU | 390 | CG2 | ILE A | 94 | 8160 | 4888 | 6417 | −2339 | −109 | | | −79 | A | C |
| ATOM | 391 | CD1 | ILE A | 94 | 35.281 | 8.828 | 42.357 | | | 1.00 | 50.65 | | A | C |
| ANISOU | 391 | CD1 | ILE A | 94 | 8079 | 4771 | 6395 | −1977 | −55 | | | −290 | A | C |
| ATOM | 392 | N | GLU A | 95 | 29.800 | 6.815 | 42.980 | | | 1.00 | 64.94 | | A | N |
| ANISOU | 392 | N | GLU A | 95 | 9539 | 6966 | 8170 | −2831 | −189 | | | 218 | A | N |
| ATOM | 393 | CA | GLU A | 95 | 28.798 | 6.213 | 43.857 | | | 1.00 | 80.14 | | A | C |
| ANISOU | 393 | CA | GLU A | 95 | 11343 | 8989 | 10116 | −2973 | −147 | | | 385 | A | C |
| ATOM | 394 | C | GLU A | 95 | 29.319 | 5.760 | 45.221 | | | 1.00 | 81.49 | | A | C |
| ANISOU | 394 | C | GLU A | 95 | 11677 | 9023 | 10261 | −2960 | −115 | | | 413 | A | C |
| ATOM | 395 | O | GLU A | 95 | 28.667 | 5.979 | 46.238 | | | 1.00 | 85.23 | | A | O |
| ANISOU | 395 | O | GLU A | 95 | 12070 | 9574 | 10741 | −3068 | −60 | | | 565 | A | O |
| ATOM | 396 | CB | GLU A | 95 | 28.083 | 5.057 | 43.145 | | | 1.00 | 90.92 | | A | C |
| ANISOU | 396 | CB | GLU A | 95 | 12606 | 10435 | 11504 | −3177 | −250 | | | 490 | A | C |
| ATOM | 397 | CG | GLU A | 95 | 29.007 | 3.977 | 42.603 | | | 1.00 | 99.86 | | A | C |
| ANISOU | 397 | CG | GLU A | 95 | 13949 | 11395 | 12600 | −3245 | −393 | | | 427 | A | C |
| ATOM | 398 | CD | GLU A | 95 | 28.267 | 2.922 | 41.804 | | | 1.00 | 107.46 | | A | C |
| ANISOU | 398 | CD | GLU A | 95 | 14812 | 12431 | 13587 | −3446 | −519 | | | 521 | A | C |
| ATOM | 399 | OE1 | GLU A | 95 | 28.832 | 1.828 | 41.595 | | | 1.00 | 116.17 | | A | O |
| ANISOU | 399 | OE1 | GLU A | 95 | 15891 | 13527 | 14721 | −3602 | −558 | | | 652 | A | O |
| ATOM | 400 | OE2 | GLU A | 95 | 27.121 | 3.188 | 41.384 | | | 1.00 | 104.53 | | A | O1− |
| ANISOU | 400 | OE2 | GLU A | 95 | 14386 | 12124 | 13207 | −3450 | −584 | | | 473 | A | O1− |
| ATOM | 401 | N | ALA A | 96 | 30.494 | 5.140 | 45.246 | | | 1.00 | 71.81 | | A | N |
| ANISOU | 401 | N | ALA A | 96 | 10668 | 7610 | 9009 | −2822 | −142 | | | 283 | A | N |
| ATOM | 402 | CA | ALA A | 96 | 31.051 | 4.670 | 46.505 | | | 1.00 | 66.37 | | A | C |
| ANISOU | 402 | CA | ALA A | 96 | 10141 | 6778 | 8297 | −2792 | −130 | | | 302 | A | C |
| ATOM | 403 | C | ALA A | 96 | 32.259 | 5.498 | 46.890 | | | 1.00 | 73.06 | | A | C |
| ANISOU | 403 | C | ALA A | 96 | 11011 | 7611 | 9137 | −2635 | −20 | | | 285 | A | C |
| ATOM | 404 | O | ALA A | 96 | 33.271 | 5.502 | 46.198 | | | 1.00 | 77.47 | | A | O |
| ANISOU | 404 | O | ALA A | 96 | 11602 | 8162 | 9669 | −2679 | 35 | | | 389 | A | O |
| ATOM | 405 | CE | ALA A | 96 | 31.432 | 3.205 | 46.396 | | | 1.00 | 57.47 | | A | C |
| ANISOU | 405 | CB | ALA A | 96 | 9223 | 5461 | 7152 | −2715 | −220 | | | 181 | A | C |
| ATOM | 406 | N | GLU A | 97 | 32.150 | 6.179 | 48.020 | | | 1.00 | 65.72 | | A | N |
| ANISOU | 406 | N | GLU A | 97 | 10071 | 6673 | 8227 | −2454 | 7 | | | 163 | A | N |
| ATOM | 407 | CA | GLU A | 97 | 33.216 | 7.029 | 48.520 | | | 1.00 | 56.65 | | A | C |
| ANISOU | 407 | CA | GLU A | 97 | 8947 | 5493 | 7085 | −2284 | 86 | | | 124 | A | C |
| ATOM | 408 | C | GLU A | 97 | 34.472 | 6.245 | 48.879 | | | 1.00 | 47.54 | | A | C |
| ANISOU | 408 | C | GLU A | 97 | 7976 | 4162 | 5925 | −2141 | 60 | | | 69 | A | C |
| ATOM | 409 | O | GLU A | 97 | 35.585 | 6.678 | 48.606 | | | 1.00 | 40.15 | | A | O |
| ANISOU | 409 | O | GLU A | 97 | 7070 | 3156 | 5028 | −1933 | 57 | | | −37 | A | O |
| ATOM | 410 | CB | GLU A | 97 | 32.707 | 7.816 | 49.727 | | | 1.00 | 59.24 | | A | C |
| ANISOU | 410 | CB | GLU A | 97 | 9191 | 5939 | 7380 | −2374 | 190 | | | 256 | A | C |
| ATOM | 411 | CG | GLU A | 97 | 33.656 | 8.866 | 50.273 | | | 1.00 | 74.07 | | A | C |
| ANISOU | 411 | CG | GLU A | 97 | 11099 | 7784 | 9260 | −2198 | 263 | | | 197 | A | C |
| ATOM | 412 | CD | GLU A | 97 | 32.990 | 9.742 | 51.316 | | | 1.00 | 93.12 | | A | C |
| ANISOU | 412 | CD | GLU A | 97 | 13369 | 10401 | 11610 | −2149 | 375 | | | 313 | A | C |
| ATOM | 413 | OE1 | GLU A | 97 | 33.630 | 10.058 | 52.340 | | | 1.00 | 90.17 | | A | O |
| ANISOU | 413 | OE1 | GLU A | 97 | 13062 | 9999 | 11198 | −1945 | 409 | | | 281 | A | O |
| ATOM | 414 | OE2 | GLU A | 97 | 31.814 | 10.105 | 51.116 | | | 1.00 | 106.91 | | A | O1− |
| ANISOU | 414 | OE2 | GLU A | 97 | 14933 | 12344 | 13343 | −2296 | 425 | | | 438 | A | O1− |
| ATOM | 415 | N | ASP A | 98 | 34.280 | 5.082 | 49.489 | | | 1.00 | 53.16 | | A | N |
| ANISOU | 415 | N | ASP A | 98 | 8787 | 4812 | 6599 | −2250 | 38 | | | 154 | A | N |
| ATOM | 416 | CA | ASP A | 98 | 35.390 | 4.249 | 49.941 | | | 1.00 | 65.58 | | A | C |
| ANISOU | 416 | CA | ASP A | 98 | 10522 | 6225 | 8169 | −2122 | 8 | | | 115 | A | C |
| ATOM | 417 | C | ASP A | 98 | 35.971 | 3.263 | 48.934 | | | 1.00 | 61.88 | | A | C |
| ANISOU | 417 | C | ASP A | 98 | 10134 | 5649 | 7730 | −2056 | −72 | | | 16 | A | C |
| ATOM | 418 | O | ASP A | 98 | 36.968 | 2.610 | 49.224 | | | 1.00 | 67.72 | | A | O |
| ANISOU | 418 | O | ASP A | 98 | 10968 | 6271 | 8490 | −1905 | −91 | | | −38 | A | O |
| ATOM | 419 | CB | ASP A | 98 | 34.987 | 3.496 | 51.210 | | | 1.00 | 77.98 | | A | C |
| ANISOU | 419 | CB | ASP A | 98 | 12180 | 7762 | 9687 | −2261 | 17 | | | 255 | A | C |
| ATOM | 420 | CG | ASP A | 98 | 34.719 | 4.424 | 52.374 | | | 1.00 | 93.26 | | A | C |
| ANISOU | 420 | CG | ASP A | 98 | 14093 | 9774 | 11568 | −2275 | 121 | | | 355 | A | C |
| ATOM | 421 | OD1 | ASP A | 98 | 35.651 | 5.141 | 52.792 | | | 1.00 | 89.20 | | A | O |
| ANISOU | 421 | OD1 | ASP A | 98 | 13623 | 9216 | 11053 | −2087 | 150 | | | 283 | A | O |
| ATOM | 422 | OD2 | ASP A | 98 | 33.577 | 4.432 | 52.875 | | | 1.00 | 107.87 | | A | O1− |
| ANISOU | 422 | OD2 | ASP A | 98 | 15873 | 11736 | 13375 | −2469 | 177 | | | 515 | A | O1− |
| ATOM | 423 | N | LYS A | 99 | 35.363 | 3.139 | 47.762 | | | 1.00 | 62.44 | | A | N |
| ANISOU | 423 | N | LYS A | 99 | 10169 | 5757 | 7797 | −2162 | −118 | | | −5 | A | N |
| ATOM | 424 | CA | LYS A | 99 | 35.872 | 2.193 | 46.776 | | | 1.00 | 64.48 | | A | C |
| ANISOU | 424 | CA | LYS A | 99 | 10545 | 5897 | 8056 | −2122 | −190 | | | −90 | A | C |
| ATOM | 425 | C | LYS A | 99 | 37.267 | 2.572 | 46.296 | | | 1.00 | 57.96 | | A | C |
| ANISOU | 425 | C | LYS A | 99 | 9725 | 5026 | 7273 | −1878 | −160 | | | −214 | A | C |
| ATOM | 426 | O | LYS A | 99 | 37.553 | 3.743 | 46.071 | | | 1.00 | 56.91 | | A | O |
| ANISOU | 426 | O | LYS A | 99 | 9473 | 4980 | 7171 | −1780 | −108 | | | −248 | A | O |
| ATOM | 427 | CE | LYS A | 99 | 34.916 | 2.081 | 45.589 | | | 1.00 | 69.66 | | A | C |
| ANISOU | 427 | CB | LYS A | 99 | 11178 | 6604 | 8686 | −2286 | −253 | | | −90 | A | C |
| ATOM | 428 | CG | LYS A | 99 | 35.383 | 1.111 | 44.516 | | | 1.00 | 77.75 | | A | C |
| ANISOU | 428 | CG | LYS A | 99 | 12353 | 7501 | 9688 | −2245 | −326 | | | −189 | A | C |
| ATOM | 429 | CD | LYS A | 99 | 34.251 | 0.732 | 43.578 | | | 1.00 | 80.79 | | A | C |
| ANISOU | 429 | CD | LYS A | 99 | 12765 | 7899 | 10033 | −2455 | −426 | | | −157 | A | C |
| ATOM | 430 | CE | LYS A | 99 | 33.577 | 1.963 | 43.001 | | | 1.00 | 78.59 | | A | C |
| ANISOU | 430 | CE | LYS A | 99 | 12333 | 7779 | 9747 | −2514 | −415 | | | −154 | A | C |
| ATOM | 431 | NZ | LYS A | 99 | 32.376 | 1.606 | 42.199 | | | 1.00 | 84.63 | | A | N1+ |
| ANISOU | 431 | NZ | LYS A | 99 | 13114 | 8559 | 10481 | −2730 | −534 | | | −107 | A | N1+ |
| ATOM | 432 | N | LYS A | 100 | 38.133 | 1.577 | 46.135 | | | 1.00 | 53.53 | | A | N |
| ANISOU | 432 | N | LYS A | 100 | 9290 | 4325 | 6722 | −1782 | −193 | | | −269 | A | N |
| ATOM | 433 | CA | LYS A | 100 | 39.488 | 1.833 | 45.672 | | | 1.00 | 50.73 | | A | C |
| ANISOU | 433 | CA | LYS A | 100 | 8925 | 3928 | 6422 | −1558 | −161 | | | −368 | A | C |
| ATOM | 434 | C | LYS A | 100 | 39.471 | 2.305 | 44.219 | | | 1.00 | 58.40 | | A | C |
| ANISOU | 434 | C | LYS A | 100 | 9872 | 4945 | 7374 | −1546 | −153 | | | −444 | A | C |
| ATOM | 435 | O | LYS A | 100 | 38.823 | 1.697 | 43.357 | | | 1.00 | 61.43 | | A | O |
| ANISOU | 435 | O | LYS A | 100 | 10347 | 5301 | 7691 | −1673 | −208 | | | −462 | A | O |
| ATOM | 436 | CB | LYS A | 100 | 40.350 | 0.573 | 45.822 | | | 1.00 | 51.07 | | A | C |
| ANISOU | 436 | CB | LYS A | 100 | 9109 | 3812 | 6483 | −1476 | −196 | | | −402 | A | C |
| ATOM | 437 | CG | LYS A | 100 | 41.860 | 0.828 | 45.638 | | | 1.00 | 61.24 | | A | C |
| ANISOU | 437 | CG | LYS A | 100 | 10356 | 5060 | 7854 | −1238 | −153 | | | −478 | A | C |
| ATOM | 438 | CD | LYS A | 100 | 42.737 | −0.388 | 45.997 | | | 1.00 | 63.80 | | A | C |
| ANISOU | 438 | CD | LYS A | 100 | 10800 | 5230 | 8213 | −1154 | −183 | | | −497 | A | C |
| ATOM | 439 | CE | LYS A | 100 | 44.230 | −0.064 | 45.824 | | | 1.00 | 68.69 | | A | C |
| ANISOU | 439 | CE | LYS A | 100 | 11343 | 5821 | 8934 | −927 | −137 | | | −558 | A | C |
| ATOM | 440 | NZ | LYS A | 100 | 45.147 | −1.185 | 46.205 | | | 1.00 | 74.32 | | A | N1+ |
| ANISOU | 440 | NZ | LYS A | 100 | 12152 | 6387 | 9698 | −836 | −162 | | | −574 | A | N1+ |
| ATOM | 441 | N | LEU A | 101 | 40.185 | 3.396 | 43.954 | | | 1.00 | 53.74 | | A | N |
| ANISOU | 441 | N | LEU A | 101 | 9165 | 4416 | 6837 | −1397 | −94 | | | −485 | A | N |
| ATOM | 442 | CA | LEU A | 101 | 40.242 | 3.971 | 42.625 | | | 1.00 | 50.02 | | A | C |
| ANISOU | 442 | CA | LEU A | 101 | 8674 | 3991 | 6342 | −1378 | −75 | | | −548 | A | C |
| ATOM | 443 | C | LEU A | 101 | 41.080 | 3.102 | 41.695 | | | 1.00 | 63.10 | | A | C |
| ANISOU | 443 | C | LEU A | 101 | 10477 | 5525 | 7972 | −1286 | −80 | | | −636 | A | C |
| ATOM | 444 | O | LEU A | 101 | 41.923 | 2.307 | 42.125 | | | 1.00 | 64.95 | | A | O |
| ANISOU | 444 | O | LEU A | 101 | 10780 | 5650 | 8246 | −1182 | −81 | | | −654 | A | O |
| ATOM | 445 | CB | LEU A | 101 | 40.840 | 5.375 | 42.681 | | | 1.00 | 45.05 | | A | C |
| ANISOU | 445 | CB | LEU A | 101 | 7881 | 3446 | 5791 | −1243 | −14 | | | −555 | A | C |
| ATOM | 446 | CG | LEU A | 101 | 40.106 | 6.319 | 43.607 | | | 1.00 | 33.81 | | A | C |
| ANISOU | 446 | CG | LEU A | 101 | 6325 | 2130 | 4391 | −1307 | −4 | | | −482 | A | C |
| ATOM | 447 | CD1 | LEU A | 101 | 40.797 | 7.675 | 43.726 | | | 1.00 | 32.04 | | A | C |
| ANISOU | 447 | CD1 | LEU A | 101 | 5951 | 1962 | 4259 | −1159 | 37 | | | −492 | A | C |
| ATOM | 448 | CD2 | LEU A | 101 | 38.707 | 6.469 | 43.071 | | | 1.00 | 34.38 | | A | C |
| ANISOU | 448 | CD2 | LEU A | 101 | 6383 | 2293 | 4388 | −1515 | −30 | | | −448 | A | C |
| ATOM | 449 | N | ILE A | 102 | 40.854 | 3.279 | 40.397 | | | 1.00 | 59.53 | | A | N |
| ANISOU | 449 | N | ILE A | 102 | 10082 | 5090 | 7448 | −1321 | −80 | | | −691 | A | N |
| ATOM | 450 | CA | ILE A | 102 | 41.634 | 2.585 | 39.379 | | | 1.00 | 58.07 | | A | C |
| ANISOU | 450 | CA | ILE A | 102 | 10060 | 4792 | 7212 | −1223 | −68 | | | −785 | A | C |
| ATOM | 451 | C | ILE A | 102 | 42.781 | 3.510 | 38.982 | | | 1.00 | 58.03 | | A | C |
| ANISOU | 451 | C | ILE A | 102 | 9969 | 4811 | 7268 | −1030 | 30 | | | −822 | A | C |
| ATOM | 452 | O | ILE A | 102 | 42.587 | 4.736 | 38.963 | | | 1.00 | 55.93 | | A | O |
| ANISOU | 452 | O | ILE A | 102 | 9555 | 4660 | 7038 | −1035 | 64 | | | −790 | A | O |
| ATOM | 453 | CE | ILE A | 102 | 40.756 | 2.202 | 38.180 | | | 1.00 | 56.86 | | A | C |
| ANISOU | 453 | CB | ILE A | 102 | 10051 | 4629 | 6925 | −1369 | −131 | | | −827 | A | C |
| ATOM | 454 | CG1 | ILE A | 102 | 39.584 | 1.341 | 38.647 | | | 1.00 | 59.34 | | A | C |
| ANISOU | 454 | CG1 | ILE A | 102 | 10422 | 4920 | 7206 | −1576 | −241 | | | −767 | A | C |
| ATOM | 455 | CG2 | ILE A | 102 | 41.570 | 1.482 | 37.121 | | | 1.00 | 62.57 | | A | C |
| ANISOU | 455 | CG2 | ILE A | 102 | 10977 | 5226 | 7572 | −1251 | −113 | | | −935 | A | C |
| ATOM | 456 | CD1 | ILE A | 102 | 38.730 | 0.817 | 37.518 | | | 1.00 | 66.14 | | A | C |
| ANISOU | 456 | CD1 | ILE A | 102 | 11434 | 5751 | 7947 | −1728 | −338 | | | −803 | A | C |
| ATOM | 457 | N | PRO A | 103 | 43.990 | 2.997 | 38.722 | | | 1.00 | 62.33 | | A | N |
| ANISOU | 457 | N | PRO A | 103 | 10592 | 5251 | 7842 | −856 | 78 | | | −879 | A | N |
| ATOM | 458 | CA | PRO A | 103 | 45.060 | 3.871 | 38.211 | | | 1.00 | 54.11 | | A | C |
| ANISOU | 458 | CA | PRO A | 103 | 9475 | 4227 | 6857 | −680 | 178 | | | −903 | A | C |
| ATOM | 459 | C | PRO A | 103 | 44.620 | 4.601 | 36.956 | | | 1.00 | 56.11 | | A | C |
| ANISOU | 459 | C | PRO A | 103 | 9772 | 4543 | 7004 | −730 | 218 | | | −938 | A | C |
| ATOM | 460 | O | PRO A | 103 | 44.032 | 4.011 | 36.047 | | | 1.00 | 59.88 | | A | O |
| ANISOU | 460 | O | PRO A | 103 | 10428 | 4987 | 7336 | −821 | 184 | | | −995 | A | O |
| ATOM | 461 | CB | PRO A | 103 | 46.213 | 2.904 | 37.930 | | | 1.00 | 49.39 | | A | C |
| ANISOU | 461 | CB | PRO A | 103 | 9005 | 3489 | 6270 | −514 | 218 | | | −965 | A | C |
| ATOM | 462 | CG | PRO A | 103 | 46.023 | 1.817 | 38.949 | | | 1.00 | 56.93 | | A | C |
| ANISOU | 462 | CG | PRO A | 103 | 10002 | 4371 | 7259 | −570 | 133 | | | −941 | A | C |
| ATOM | 463 | CD | PRO A | 103 | 44.512 | 1.683 | 39.161 | | | 1.00 | 64.17 | | A | C |
| ANISOU | 463 | CD | PRO A | 103 | 10945 | 5346 | 8093 | −802 | 45 | | | −901 | A | C |
| ATOM | 464 | N | ASP A | 104 | 44.870 | 5.910 | 36.938 | | | 1.00 | 57.11 | | A | N |
| ANISOU | 464 | N | ASP A | 104 | 9737 | 4757 | 7204 | −680 | 278 | | | −900 | A | N |
| ATOM | 465 | CA | ASP A | 104 | 44.496 | 6.860 | 35.882 | | | 1.00 | 57.32 | | A | C |
| ANISOU | 465 | CA | ASP A | 104 | 9768 | 4856 | 7155 | −728 | 327 | | | −916 | A | C |
| ATOM | 466 | C | ASP A | 104 | 43.014 | 7.218 | 35.823 | | | 1.00 | 47.29 | | A | C |
| ANISOU | 466 | C | ASP A | 104 | 8460 | 3693 | 5815 | −954 | 239 | | | −885 | A | C |
| ATOM | 467 | O | ASP A | 104 | 42.633 | 7.998 | 34.934 | | | 1.00 | 43.77 | | A | O |
| ANISOU | 467 | O | ASP A | 104 | 8017 | 3311 | 5303 | −1015 | 262 | | | −897 | A | O |
| ATOM | 468 | CB | ASP A | 104 | 44.913 | 6.385 | 34.494 | | | 1.00 | 72.35 | | A | C |
| ANISOU | 468 | CB | ASP A | 104 | 11894 | 6684 | 8912 | −655 | 398 | | | −1009 | A | C |
| ATOM | 469 | CG | ASP A | 104 | 45.339 | 7.524 | 33.610 | | | 1.00 | 89.28 | | A | C |
| ANISOU | 469 | CG | ASP A | 104 | 14010 | 8870 | 11043 | −575 | 521 | | | −1013 | A | C |
| ATOM | 470 | OD1 | ASP A | 104 | 45.462 | 8.663 | 34.119 | | | 1.00 | 92.67 | | A | O |
| ANISOU | 470 | OD1 | ASP A | 104 | 14233 | 9368 | 11612 | −563 | 547 | | | −939 | A | O |
| ATOM | 471 | OD2 | ASP A | 104 | 45.546 | 7.272 | 32.402 | | | 1.00 | 100.17 | | A | O1− |
| ANISOU | 471 | OD2 | ASP A | 104 | 15584 | 10208 | 12267 | −519 | 594 | | | −1089 | A | O1− |
| ATOM | 472 | N | GLN A | 105 | 42.176 | 6.695 | 36.724 | | | 1.00 | 48.14 | | A | N |
| ANISOU | 472 | N | GLN A | 105 | 8532 | 3823 | 5938 | −1081 | 146 | | | −839 | A | N |
| ATOM | 473 | CA | GLN A | 105 | 40.797 | 7.148 | 36.847 | | | 1.00 | 35.73 | | A | C |
| ANISOU | 473 | CA | GLN A | 105 | 6874 | 2367 | 4335 | −1282 | 75 | | | −785 | A | C |
| ATOM | 474 | C | GLN A | 105 | 40.753 | 8.656 | 37.058 | | | 1.00 | 42.95 | | A | C |
| ANISOU | 474 | C | GLN A | 105 | 7585 | 3397 | 5338 | −1264 | 117 | | | −736 | A | C |
| ATOM | 475 | O | GLN A | 105 | 41.622 | 9.230 | 37.714 | | | 1.00 | 42.73 | | A | O |
| ANISOU | 475 | O | GLN A | 105 | 7445 | 3358 | 5433 | −1116 | 168 | | | −714 | A | O |
| ATOM | 476 | CE | GLN A | 105 | 40.128 | 6.398 | 37.993 | | | 1.00 | 36.09 | | A | C |
| ANISOU | 476 | CB | GLN A | 105 | 6899 | 2405 | 4408 | −1379 | 5 | | | −727 | A | C |
| ATOM | 477 | CG | GLN A | 105 | 38.808 | 6.935 | 38.518 | | | 1.00 | 42.58 | | A | C |
| ANISOU | 477 | CG | GLN A | 105 | 7584 | 3354 | 5239 | −1557 | −44 | | | −644 | A | C |
| ATOM | 478 | CD | GLN A | 105 | 38.114 | 5.902 | 39.409 | | | 1.00 | 51.85 | | A | C |
| ANISOU | 478 | CD | GLN A | 105 | 8806 | 4491 | 6405 | −1675 | −107 | | | −590 | A | C |
| ATOM | 479 | OE1 | GLN A | 105 | 38.721 | 4.902 | 39.815 | | | 1.00 | 64.31 | | A | O |
| ANISOU | 479 | OE1 | GLN A | 105 | 10497 | 5949 | 7990 | −1606 | −113 | | | −611 | A | O |
| ATOM | 480 | NE2 | GLN A | 105 | 36.843 | 6.131 | 39.702 | | | 1.00 | 50.34 | | A | N |
| ANISOU | 480 | NE2 | GLN A | 105 | 8523 | 4401 | 6202 | −1856 | −153 | | | −512 | A | N |
| ATOM | 481 | N | LEU A | 106 | 39.759 | 9.310 | 36.461 | | | 1.00 | 46.07 | | A | N |
| ANISOU | 481 | N | LEU A | 106 | 5733 | 6445 | 5326 | −922 | 1721 | | | −1470 | A | N |
| ATOM | 482 | CA | LEU A | 106 | 39.670 | 10.766 | 36.469 | | | 1.00 | 45.16 | | A | C |
| ANISOU | 482 | CA | LEU A | 106 | 5498 | 6507 | 5154 | −817 | 1620 | | | −1382 | A | C |
| ATOM | 483 | C | LEU A | 106 | 38.638 | 11.233 | 37.498 | | | 1.00 | 44.35 | | A | C |
| ANISOU | 483 | C | LEU A | 106 | 5430 | 6310 | 5112 | −864 | 1605 | | | −1495 | A | C |
| ATOM | 484 | O | LEU A | 106 | 37.521 | 10.699 | 37.566 | | | 1.00 | 48.19 | | A | O |
| ANISOU | 484 | O | LEU A | 106 | 5859 | 6822 | 5629 | −979 | 1618 | | | −1682 | A | O |
| ATOM | 485 | CB | LEU A | 106 | 39.337 | 11.297 | 35.068 | | | 1.00 | 33.66 | | A | C |
| ANISOU | 485 | CE | LEU A | 106 | 3753 | 5467 | 3571 | −757 | 1522 | | | −1398 | A | C |
| ATOM | 486 | CG | LEU A | 106 | 39.301 | 12.816 | 34.865 | | | 1.00 | 32.57 | | A | C |
| ANISOU | 486 | CG | LEU A | 106 | 3431 | 5585 | 3359 | −630 | 1422 | | | −1266 | A | C |
| ATOM | 487 | CD1 | LEU A | 106 | 40.721 | 13.418 | 34.869 | | | 1.00 | 28.83 | | A | C |
| ANISOU | 487 | CD1 | LEU A | 106 | 3030 | 4991 | 2934 | −527 | 1327 | | | −899 | A | C |
| ATOM | 488 | CD2 | LEU A | 106 | 38.534 | 13.172 | 33.570 | | | 1.00 | 31.73 | | A | C |
| ANISOU | 488 | CD2 | LEU A | 106 | 3027 | 5922 | 3107 | −568 | 1305 | | | −1329 | A | C |
| ATOM | 489 | N | LEU A | 107 | 39.033 | 12.231 | 38.296 | | | 1.00 | 35.50 | | A | N |
| ANISOU | 489 | N | LEU A | 107 | 4392 | 5082 | 4014 | −788 | 1588 | | | −1384 | A | N |
| ATOM | 490 | CA | LEU A | 107 | 38.264 | 12.808 | 39.384 | | | 1.00 | 27.85 | | A | C |
| ANISOU | 490 | CA | LEU A | 107 | 3481 | 3993 | 3106 | −803 | 1570 | | | −1474 | A | C |
| ATOM | 491 | C | LEU A | 107 | 38.275 | 14.325 | 39.239 | | | 1.00 | 27.70 | | A | C |
| ANISOU | 491 | C | LEU A | 107 | 3328 | 4085 | 3111 | −647 | 1382 | | | −1337 | A | C |
| ATOM | 492 | O | LEU A | 107 | 39.252 | 14.901 | 38.777 | | | 1.00 | 27.08 | | A | O |
| ANISOU | 492 | O | LEU A | 107 | 3230 | 3992 | 3069 | −531 | 1219 | | | −1034 | A | O |
| ATOM | 493 | CB | LEU A | 107 | 38.867 | 12.458 | 40.767 | | | 1.00 | 38.01 | | A | C |
| ANISOU | 493 | CB | LEU A | 107 | 5071 | 4902 | 4469 | −836 | 1637 | | | −1383 | A | C |
| ATOM | 494 | CG | LEU A | 107 | 39.169 | 11.110 | 41.452 | | | 1.00 | 41.12 | | A | C |
| ANISOU | 494 | CG | LEU A | 107 | 5679 | 5036 | 4909 | −929 | 1743 | | | −1341 | A | C |
| ATOM | 495 | CD1 | LEU A | 107 | 38.079 | 10.128 | 41.233 | | | 1.00 | 48.16 | | A | C |
| ANISOU | 495 | CD1 | LEU A | 107 | 6483 | 5984 | 5831 | −1081 | 1803 | | | −1503 | A | C |
| ATOM | 496 | CD2 | LEU A | 107 | 40.473 | 10.528 | 41.027 | | | 1.00 | 50.97 | | A | C |
| ANISOU | 496 | CD2 | LEU A | 107 | 7032 | 6202 | 6133 | −870 | 1796 | | | −1195 | A | C |
| ATOM | 497 | N | LEU A | 108 | 37.223 | 14.993 | 39.696 | | | 1.00 | 32.50 | | A | N |
| ANISOU | 497 | N | LEU A | 108 | 3854 | 4741 | 3752 | −622 | 1327 | | | −1482 | A | N |
| ATOM | 498 | CA | LEU A | 108 | 37.258 | 16.446 | 39.836 | | | 1.00 | 41.10 | | A | C |
| ANISOU | 498 | CA | LEU A | 108 | 4882 | 5785 | 4950 | −443 | 1073 | | | −1270 | A | C |
| ATOM | 499 | C | LEU A | 108 | 37.277 | 16.827 | 41.315 | | | 1.00 | 51.67 | | A | C |
| ANISOU | 499 | C | LEU A | 108 | 6426 | 6802 | 6406 | −461 | 1060 | | | −1285 | A | C |
| ATOM | 500 | O | LEU A | 108 | 36.597 | 16.211 | 42.142 | | | 1.00 | 46.08 | | A | C |
| ANISOU | 500 | O | LEU A | 108 | 5810 | 6032 | 5667 | −589 | 1235 | | | −1546 | A | O |
| ATOM | 501 | CB | LEU A | 108 | 36.064 | 17.126 | 39.143 | | | 1.00 | 47.11 | | A | O |
| ANISOU | 501 | CB | LEU A | 108 | 5364 | 6871 | 5666 | −333 | 978 | | | −1396 | A | C |
| ATOM | 502 | CG | LEU A | 108 | 35.942 | 17.146 | 37.607 | | | 1.00 | 42.34 | | A | C |
| ANISOU | 502 | CG | LEU A | 108 | 4504 | 6660 | 4924 | −248 | 914 | | | −1340 | A | C |
| ATOM | 503 | CD1 | LEU A | 108 | 37.252 | 17.498 | 36.938 | | | 1.00 | 30.35 | | A | C |
| ANISOU | 503 | CD1 | LEU A | 108 | 3029 | 5079 | 3421 | −163 | 793 | | | −953 | A | C |
| ATOM | 504 | CD2 | LEU A | 108 | 35.406 | 15.844 | 37.070 | | | 1.00 | 34.62 | | A | C |
| ANISOU | 504 | CD2 | LEU A | 108 | 3422 | 5948 | 3784 | −424 | 1136 | | | −1672 | A | C |
| ATOM | 505 | N | VAL A | 109 | 38.053 | 17.854 | 41.641 | | | 1.00 | 45.38 | | A | N |
| ANISOU | 505 | N | VAL A | 109 | 5695 | 5812 | 5737 | −346 | 866 | | | −1015 | A | N |
| ATOM | 506 | CA | VAL A | 109 | 38.298 | 18.298 | 43.005 | | | 1.00 | 27.02 | | A | C |
| ANISOU | 506 | CA | VAL A | 109 | 3556 | 3193 | 3516 | −356 | 827 | | | −1006 | A | C |
| ATOM | 507 | C | VAL A | 109 | 37.893 | 19.762 | 43.087 | | | 1.00 | 35.67 | | A | C |
| ANISOU | 507 | C | VAL A | 109 | 4551 | 4250 | 4753 | −209 | 629 | | | −941 | A | C |
| ATOM | 508 | O | VAL A | 109 | 38.447 | 20.590 | 42.355 | | | 1.00 | 46.49 | | A | O |
| ANISOU | 508 | O | VAL A | 109 | 5842 | 5621 | 6199 | −93 | 468 | | | −681 | A | O |
| ATOM | 509 | CB | VAL A | 109 | 39.777 | 18.140 | 43.401 | | | 1.00 | 25.46 | | A | C |
| ANISOU | 509 | CB | VAL A | 109 | 3538 | 2782 | 3352 | −370 | 783 | | | −763 | A | C |
| ATOM | 510 | CG1 | VAL A | 109 | 40.045 | 18.740 | 44.795 | | | 1.00 | 25.30 | | A | C |
| ANISOU | 510 | CG1 | VAL A | 109 | 3680 | 2507 | 3425 | −370 | 711 | | | −761 | A | C |
| ATOM | 511 | CG2 | VAL A | 109 | 40.202 | 16.677 | 43.298 | | | 1.00 | 24.96 | | A | C |
| ANISOU | 511 | CG2 | VAL A | 109 | 3589 | 2732 | 3161 | −475 | 985 | | | −811 | A | C |
| ATOM | 512 | N | PRO A | 110 | 36.975 | 20.132 | 43.979 | | | 1.00 | 27.98 | | A | N |
| ANISOU | 512 | N | PRO A | 110 | 3588 | 3220 | 3825 | −208 | 648 | | | −1163 | A | N |
| ATOM | 513 | CA | PRO A | 110 | 36.577 | 21.539 | 44.103 | | | 1.00 | 29.10 | | A | C |
| ANISOU | 513 | CA | PRO A | 110 | 3645 | 3287 | 4124 | −47 | 472 | | | −1118 | A | C |
| ATOM | 514 | C | PRO A | 110 | 37.703 | 22.399 | 44.651 | | | 1.00 | 32.87 | | A | C |
| ANISOU | 514 | C | PRO A | 110 | 4264 | 3463 | 4760 | −18 | 329 | | | −883 | A | C |
| ATOM | 515 | O | PRO A | 110 | 38.524 | 21.949 | 45.455 | | | 1.00 | 37.50 | | A | O |
| ANISOU | 515 | O | PRO A | 110 | 5028 | 3894 | 5327 | −130 | 371 | | | −860 | A | O |
| ATOM | 516 | CB | PRO A | 110 | 35.392 | 21.492 | 45.068 | | | 1.00 | 29.83 | | A | C |
| ANISOU | 516 | CB | PRO A | 110 | 3732 | 3396 | 4207 | −88 | 571 | | | −1463 | A | C |
| ATOM | 517 | CG | PRO A | 110 | 35.018 | 20.051 | 45.153 | | | 1.00 | 31.54 | | A | C |
| ANISOU | 517 | CG | PRO A | 110 | 3985 | 3754 | 4243 | −275 | 809 | | | −1680 | A | C |
| ATOM | 518 | CD | PRO A | 110 | 36.231 | 19.260 | 44.888 | | | 1.00 | 31.02 | | A | C |
| ANISOU | 518 | CD | PRO A | 110 | 4063 | 3604 | 4118 | −357 | 854 | | | −1471 | A | C |
| ATOM | 519 | N | VAL A | 111 | 37.716 | 23.665 | 44.229 | | | 1.00 | 29.92 | | A | N |
| ANISOU | 519 | N | VAL A | 111 | 3811 | 3012 | 4546 | 138 | 163 | | | −717 | A | N |
| ATOM | 520 | CA | VAL A | 111 | 38.888 | 24.514 | 44.406 | | | 1.00 | 33.23 | | A | C |
| ANISOU | 520 | CA | VAL A | 111 | 4328 | 3171 | 5128 | 145 | 34 | | | −463 | A | C |
| ATOM | 521 | C | VAL A | 111 | 38.458 | 25.973 | 44.336 | | | 1.00 | 35.97 | | A | C |
| ANISOU | 521 | C | VAL A | 111 | 4620 | 3363 | 5684 | 312 | −99 | | | −400 | A | C |
| ATOM | 522 | O | VAL A | 111 | 37.504 | 26.324 | 43.642 | | | 1.00 | 41.50 | | A | O |
| ANISOU | 522 | O | VAL A | 111 | 5173 | 4216 | 6378 | 475 | −127 | | | −418 | A | O |
| ATOM | 523 | CB | VAL A | 111 | 39.963 | 24.177 | 43.338 | | | 1.00 | 29.33 | | A | C |
| ANISOU | 523 | CB | VAL A | 111 | 3807 | 2763 | 4576 | 120 | 13 | | | −168 | A | C |
| ATOM | 524 | CG1 | VAL A | 111 | 40.603 | 25.421 | 42.830 | | | 1.00 | 30.58 | | A | C |
| ANISOU | 524 | CG1 | VAL A | 111 | 3947 | 2759 | 4915 | 205 | −131 | | | 123 | A | C |
| ATOM | 525 | CG2 | VAL A | 111 | 40.987 | 23.211 | 43.889 | | | 1.00 | 27.76 | | A | C |
| ANISOU | 525 | CG2 | VAL A | 111 | 3744 | 2517 | 4288 | −34 | 88 | | | −161 | A | C |
| ATOM | 526 | N | THR A | 112 | 39.187 | 26.832 | 45.045 | | | 1.00 | 38.61 | | A | N |
| ANISOU | 526 | N | THR A | 112 | 5070 | 3394 | 6205 | 280 | −180 | | | −328 | A | N |
| ATOM | 527 | CA | THR A | 112 | 38.872 | 28.253 | 45.133 | | | 1.00 | 39.76 | | A | C |
| ANISOU | 527 | CA | THR A | 112 | 5207 | 3308 | 6591 | 422 | −284 | | | −285 | A | C |
| ATOM | 528 | C | THR A | 112 | 39.954 | 29.026 | 44.388 | | | 1.00 | 46.00 | | A | C |
| ANISOU | 528 | C | THR A | 112 | 6022 | 3929 | 7525 | 429 | −371 | | | 75 | A | C |
| ATOM | 529 | O | THR A | 112 | 41.116 | 29.032 | 44.810 | | | 1.00 | 56.96 | | A | O |
| ANISOU | 529 | O | THR A | 112 | 7504 | 5173 | 8966 | 269 | −385 | | | 152 | A | O |
| ATOM | 530 | CB | THR A | 112 | 38.767 | 28.688 | 46.598 | | | 1.00 | 39.12 | | A | C |
| ANISOU | 530 | CB | THR A | 112 | 5235 | 2999 | 6628 | 355 | −283 | | | −544 | A | C |
| ATOM | 531 | OG1 | THR A | 112 | 37.807 | 27.858 | 47.275 | | | 1.00 | 37.11 | | A | O |
| ANISOU | 531 | OG1 | THR A | 112 | 4964 | 2926 | 6208 | 319 | −173 | | | −866 | A | O |
| ATOM | 532 | CG2 | THR A | 112 | 38.343 | 30.153 | 46.721 | | | 1.00 | 37.45 | | A | C |
| ANISOU | 532 | CG2 | THR A | 112 | 5019 | 2520 | 6689 | 512 | −369 | | | −545 | A | C |
| ATOM | 533 | N | CYS A | 113 | 39.554 | 29.726 | 43.335 | | | 1.00 | 48.00 | | A | N |
| ANISOU | 533 | N | CYS A | 113 | 6185 | 4232 | 7823 | 613 | −422 | | | 295 | A | N |
| ATOM | 534 | CA | CYS A | 113 | 40.479 | 30.540 | 42.572 | | | 1.00 | 53.06 | | A | C |
| ANISOU | 534 | CA | CYS A | 113 | 6860 | 4680 | 8618 | 637 | −485 | | | 656 | A | C |
| ATOM | 535 | C | CYS A | 113 | 40.556 | 31.933 | 43.138 | | | 1.00 | 66.90 | | A | C |
| ANISOU | 535 | C | CYS A | 113 | 8725 | 6007 | 10686 | 660 | −535 | | | 645 | A | C |
| ATOM | 536 | O | CYS A | 113 | 39.559 | 32.498 | 43.578 | | | 1.00 | 75.07 | | A | O |
| ANISOU | 536 | O | CYS A | 113 | 9761 | 6934 | 11827 | 790 | −548 | | | 429 | A | O |
| ATOM | 537 | CB | CYS A | 113 | 39.975 | 30.766 | 41.153 | | | 1.00 | 56.88 | | A | C |
| ANISOU | 537 | CB | CYS A | 113 | 7227 | 5357 | 9029 | 859 | −518 | | | 909 | A | C |
| ATOM | 538 | SG | CYS A | 113 | 40.313 | 29.552 | 39.874 | | | 1.00 | 71.14 | | A | S |
| ANISOU | 538 | SG | CYS A | 113 | 8928 | 7527 | 10573 | 819 | −489 | | | 1204 | A | S |
| ATOM | 539 | N | GLY A | 114 | 41.757 | 32.479 | 43.130 | | | 1.00 | 72.53 | | A | N |
| ANISOU | 539 | N | GLY A | 114 | 9521 | 6480 | 11556 | 525 | −551 | | | 864 | A | N |
| ATOM | 540 | CA | GLY A | 114 | 41.958 | 33.868 | 43.477 | | | 1.00 | 74.83 | | A | C |
| ANISOU | 540 | CA | GLY A | 114 | 9917 | 6341 | 12174 | 542 | −580 | | | 920 | A | C |
| ATOM | 541 | C | GLY A | 114 | 42.963 | 34.456 | 42.510 | | | 1.00 | 70.71 | | A | C |
| ANISOU | 541 | C | GLY A | 114 | 9428 | 5734 | 11704 | 476 | −563 | | | 1297 | A | C |
| ATOM | 542 | O | GLY A | 114 | 43.636 | 33.728 | 41.778 | | | 1.00 | 57.89 | | A | O |
| ANISOU | 542 | O | GLY A | 114 | 7742 | 4320 | 9933 | 391 | −552 | | | 1497 | A | O |
| ATOM | 543 | N | CYS A | 115 | 43.081 | 35.769 | 42.570 | | | 1.00 | 68.06 | | A | N |
| ANISOU | 543 | N | CYS A | 115 | 9195 | 5144 | 11522 | 507 | −534 | | | 1363 | A | N |
| ATOM | 544 | CA | CYS A | 115 | 43.999 | 36.467 | 41.720 | | | 1.00 | 84.86 | | A | C |
| ANISOU | 544 | CA | CYS A | 115 | 11377 | 7210 | 13658 | 420 | −482 | | | 1671 | A | C |
| ATOM | 545 | C | CYS A | 115 | 45.105 | 37.110 | 42.523 | | | 1.00 | 97.68 | | A | C |
| ANISOU | 545 | C | CYS A | 115 | 13086 | 8618 | 15410 | 150 | −434 | | | 1525 | A | C |
| ATOM | 546 | O | CYS A | 115 | 44.857 | 37.977 | 43.355 | | | 1.00 | 99.49 | | A | O |
| ANISOU | 546 | O | CYS A | 115 | 13406 | 8595 | 15802 | 153 | −421 | | | 1321 | A | O |
| ATOM | 547 | CB | CYS A | 115 | 43.267 | 37.545 | 40.946 | | | 1.00 | 91.36 | | A | C |
| ANISOU | 547 | CB | CYS A | 115 | 12267 | 7904 | 14540 | 682 | −475 | | | 1914 | A | C |
| ATOM | 548 | SG | CYS A | 115 | 44.168 | 38.093 | 39.493 | | | 1.00 | 103.41 | | A | S |
| ANISOU | 548 | SG | CYS A | 115 | 13861 | 9421 | 16009 | 610 | −402 | | | 2344 | A | S |
| ATOM | 549 | N | THR A | 116 | 46.326 | 36.653 | 42.284 | | | 1.00 | 98.83 | | A | N |
| ANISOU | 549 | N | THR A | 116 | 13185 | 8890 | 15476 | −78 | −410 | | | 1608 | A | N |
| ATOM | 550 | CA | THR A | 116 | 47.507 | 37.227 | 42.923 | | | 1.00 | 104.76 | | A | C |
| ANISOU | 55C | CA | THR A | 116 | 13978 | 9488 | 16339 | −342 | −367 | | | 1503 | A | C |
| ATOM | 551 | C | THR A | 116 | 48.481 | 37.706 | 41.851 | | | 1.00 | 101.85 | | A | C |
| ANISOU | 551 | C | THR A | 116 | 13620 | 9100 | 15978 | −449 | −295 | | | 1812 | A | C |
| ATOM | 552 | O | THR A | 116 | 48.846 | 36.945 | 40.945 | | | 1.00 | 100.19 | | A | O |
| ANISOU | 552 | O | THR A | 116 | 13315 | 9149 | 15601 | −449 | −293 | | | 2019 | A | O |
| ATOM | 553 | CB | THR A | 116 | 48.202 | 36.224 | 43.872 | | | 1.00 | 103.62 | | A | C |
| ANISOU | 553 | CB | THR A | 116 | 13745 | 9527 | 16099 | −529 | −411 | | | 1265 | A | C |
| ATOM | 554 | OG1 | THR A | 116 | 48.492 | 35.001 | 43.183 | | | 1.00 | 101.20 | | A | O |
| ANISOU | 554 | OG1 | THR A | 116 | 13329 | 9526 | 15595 | −524 | −431 | | | 1417 | A | O |
| ATOM | 555 | CG2 | THR A | 116 | 47.326 | 35.926 | 45.092 | | | 1.00 | 101.92 | | A | C |
| ANISOU | 555 | CG2 | THR A | 116 | 13550 | 9297 | 15879 | −461 | −461 | | | 925 | A | C |
| ATOM | 556 | N | LYS A | 117 | 48.876 | 38.976 | 41.945 | | | 1.00 | 106.96 | | A | N |
| ANISOU | 556 | N | LYS A | 117 | 14390 | 9437 | 16814 | −544 | −224 | | | 1832 | A | N |
| ATOM | 557 | CA | LYS A | 117 | 49.850 | 39.591 | 41.034 | | | 1.00 | 107.18 | | A | C |
| ANISOU | 557 | CA | LYS A | 117 | 14456 | 9391 | 16876 | −684 | −131 | | | 2097 | A | C |
| ATOM | 558 | C | LYS A | 117 | 49.500 | 39.329 | 39.566 | | | 1.00 | 104.52 | | A | C |
| ANISOU | 558 | C | LYS A | 117 | 14101 | 9234 | 16377 | −498 | −118 | | | 2459 | A | C |
| ATOM | 559 | O | LYS A | 117 | 50.333 | 38.896 | 38.769 | | | 1.00 | 113.63 | | A | O |
| ANISOU | 559 | O | LYS A | 117 | 15172 | 10593 | 17409 | −608 | −80 | | | 2646 | A | O |
| ATOM | 560 | CB | LYS A | 117 | 51.271 | 39.117 | 41.366 | | | 1.00 | 101.15 | | A | C |
| ANISOU | 560 | CB | LYS A | 117 | 13574 | 8768 | 16089 | −989 | −114 | | | 2007 | A | C |
| ATOM | 561 | CG | LYS A | 117 | 51.846 | 39.752 | 42.638 | | | 1.00 | 97.84 | | A | C |
| ANISOU | 561 | CG | LYS A | 117 | 13190 | 8135 | 15849 | −1215 | −103 | | | 1695 | A | C |
| ATOM | 562 | CD | LYS A | 117 | 53.236 | 39.221 | 43.006 | | | 1.00 | 93.60 | | A | C |
| ANISOU | 562 | CD | LYS A | 117 | 12503 | 7781 | 15281 | −1500 | −108 | | | 1589 | A | C |
| ATOM | 563 | CE | LYS A | 117 | 54.329 | 39.840 | 42.139 | | | 1.00 | 97.27 | | A | C |
| ANISOU | 563 | CE | LYS A | 117 | 12971 | 8180 | 15808 | −1705 | 10 | | | 1808 | A | C |
| ATOM | 564 | NZ | LYS A | 117 | 55.707 | 39.616 | 42.690 | | | 1.00 | 98.21 | | A | N1+ |
| ANISOU | 564 | NZ | LYS A | 117 | 12938 | 8420 | 15955 | −2019 | 13 | | | 1641 | A | N1+ |
| ATOM | 565 | N | ASN A | 118 | 48.260 | 39.665 | 39.220 | | | 1.00 | 103.91 | | A | N |
| ANISOU | 565 | N | ASN A | 118 | 14089 | 9107 | 16286 | −203 | −154 | | | 2546 | A | N |
| ATOM | 566 | CA | ASN A | 118 | 47.709 | 39.528 | 37.868 | | | 1.00 | 91.53 | | A | C |
| ANISOU | 566 | CA | ASN A | 118 | 12514 | 7706 | 14555 | 20 | −156 | | | 2881 | A | C |
| ATOM | 567 | C | ASN A | 118 | 47.779 | 38.110 | 37.307 | | | 1.00 | 81.66 | | A | C |
| ANISOU | 567 | C | ASN A | 118 | 11074 | 6906 | 13047 | 56 | −209 | | | 2935 | A | C |
| ATOM | 568 | O | ASN A | 118 | 47.927 | 37.914 | 36.107 | | | 1.00 | 78.27 | | A | O |
| ANISOU | 568 | O | ASN A | 118 | 10608 | 6687 | 12443 | 205 | −207 | | | 3204 | A | O |
| ATOM | 569 | CB | ASN A | 118 | 48.326 | 40.557 | 36.904 | | | 1.00 | 88.71 | | A | C |
| ANISOU | 569 | CB | ASN A | 118 | 12269 | 7207 | 14229 | −76 | −48 | | | 3182 | A | C |
| ATOM | 570 | CG | ASN A | 118 | 47.423 | 40.884 | 35.736 | | | 1.00 | 86.71 | | A | C |
| ANISOU | 570 | CG | ASN A | 118 | 12133 | 6890 | 13923 | 219 | −49 | | | 3485 | A | C |
| ATOM | 571 | OD1 | ASN A | 118 | 46.834 | 39.998 | 35.121 | | | 1.00 | 81.39 | | A | O |
| ANISOU | 571 | OD1 | ASN A | 118 | 11659 | 5846 | 13421 | 263 | 6 | | | 3574 | A | O |
| ATOM | 572 | ND2 | ASN A | 118 | 47.314 | 42.168 | 35.419 | | | 1.00 | 86.46 | | A | N |
| ANISOU | 572 | ND2 | ASN A | 118 | 11978 | 7225 | 13646 | 425 | −112 | | | 3642 | A | N |
| ATOM | 573 | N | HIS A | 119 | 47.663 | 37.125 | 38.188 | | | 1.00 | 77.37 | | A | N |
| ANISOU | 573 | N | HIS A | 119 | 10415 | 6521 | 12459 | −71 | −254 | | | 2688 | A | N |
| ATOM | 574 | CA | HIS A | 119 | 47.667 | 35.725 | 37.785 | | | 1.00 | 84.38 | | A | C |
| ANISOU | 574 | CA | HIS A | 119 | 11144 | 7801 | 13114 | −30 | −298 | | | 2708 | A | C |
| ATOM | 575 | C | HIS A | 119 | 46.697 | 34.982 | 38.685 | | | 1.00 | 81.02 | | A | C |
| ANISOU | 575 | C | HIS A | 119 | 10685 | 7403 | 12697 | 54 | −376 | | | 2423 | A | C |
| ATOM | 576 | O | HIS A | 119 | 46.507 | 35.360 | 39.834 | | | 1.00 | 91.42 | | A | O |
| ANISOU | 576 | O | HIS A | 119 | 12060 | 8509 | 14165 | −43 | −390 | | | 2160 | A | O |
| ATOM | 577 | CB | HIS A | 119 | 49.065 | 35.122 | 37.878 | | | 1.00 | 90.48 | | A | C |
| ANISOU | 577 | CB | HIS A | 119 | 11816 | 8763 | 13798 | −282 | −261 | | | 2710 | A | C |
| ATOM | 578 | CG | HIS A | 119 | 49.966 | 35.507 | 36.747 | | | 1.00 | 93.10 | | A | C |
| ANISOU | 578 | CG | HIS A | 119 | 12154 | 9114 | 14107 | −387 | −173 | | | 2976 | A | C |
| ATOM | 579 | ND1 | HIS A | 119 | 50.772 | 36.623 | 36.784 | | | 1.00 | 97.41 | | A | N |
| ANISOU | 579 | ND1 | HIS A | 119 | 12820 | 9346 | 14847 | −524 | −102 | | | 3018 | A | N |
| ATOM | 580 | CD2 | HIS A | 119 | 50.191 | 34.922 | 35.548 | | | 1.00 | 93.35 | | A | C |
| ANISOU | 580 | CD2 | HIS A | 119 | 12089 | 9442 | 13938 | −387 | −132 | | | 3201 | A | C |
| ATOM | 581 | CE1 | HIS A | 119 | 51.453 | 36.711 | 35.657 | | | 1.00 | 100.49 | | A | C |
| ANISOU | 581 | CE1 | HIS A | 119 | 13193 | 9826 | 15164 | −607 | −18 | | | 3269 | A | C |
| ATOM | 582 | NE2 | HIS A | 119 | 51.118 | 35.691 | 34.889 | | | 1.00 | 97.52 | | A | N |
| ANISOU | 582 | NE2 | HIS A | 119 | 12681 | 9830 | 14544 | −520 | −39 | | | 3381 | A | N |
| ATOM | 583 | N | SER A | 120 | 46.072 | 33.931 | 38.174 | | | 1.00 | 72.27 | | A | N |
| ANISOU | 583 | N | SER A | 120 | 9477 | 6562 | 11419 | 225 | −420 | | | 2459 | A | N |
| ATOM | 584 | CA | SER A | 120 | 45.103 | 33.202 | 38.985 | | | 1.00 | 62.39 | | A | C |
| ANISOU | 584 | CA | SER A | 120 | 8200 | 5313 | 10194 | 318 | −481 | | | 2194 | A | C |
| ATOM | 585 | C | SER A | 120 | 45.565 | 31.838 | 39.499 | | | 1.00 | 52.73 | | A | C |
| ANISOU | 585 | C | SER A | 120 | 6898 | 4332 | 8805 | 172 | −476 | | | 2036 | A | C |
| ATOM | 586 | O | SER A | 120 | 46.116 | 31.044 | 38.744 | | | 1.00 | 56.94 | | A | O |
| ANISOU | 586 | O | SER A | 120 | 7346 | 5146 | 9143 | 118 | −440 | | | 2183 | A | O |
| ATOM | 587 | CB | SER A | 120 | 43.735 | 33.129 | 38.300 | | | 1.00 | 65.19 | | A | C |
| ANISOU | 587 | CB | SER A | 120 | 8490 | 5791 | 10489 | 635 | −529 | | | 2268 | A | C |
| ATOM | 588 | OG | SER A | 120 | 43.850 | 32.680 | 36.967 | | | 1.00 | 70.96 | | A | O |
| ANISOU | 588 | OG | SER A | 120 | 9106 | 6896 | 10960 | 733 | −517 | | | 2506 | A | O |
| ATOM | 589 | N | PHE A | 121 | 45.317 | 31.565 | 40.781 | | | 1.00 | 43.47 | | A | N |
| ANISOU | 589 | N | PHE A | 121 | 5764 | 3099 | 7654 | 114 | −490 | | | 1685 | A | N |
| ATOM | 590 | CA | PHE A | 121 | 45.764 | 30.314 | 41.379 | | | 1.00 | 52.78 | | A | C |
| ANISOU | 590 | CA | PHE A | 121 | 6916 | 4492 | 8646 | −22 | −464 | | | 1474 | A | C |
| ATOM | 591 | C | PHE A | 121 | 44.789 | 29.893 | 42.473 | | | 1.00 | 54.71 | | A | C |
| ANISOU | 591 | C | PHE A | 121 | 7199 | 4744 | 8844 | 28 | −464 | | | 1114 | A | C |
| ATOM | 592 | O | PHE A | 121 | 43.994 | 30.693 | 42.970 | | | 1.00 | 52.36 | | A | O |
| ANISOU | 592 | O | PHE A | 121 | 6946 | 4245 | 8704 | 125 | −493 | | | 993 | A | O |
| ATOM | 593 | CB | PHE A | 121 | 47.161 | 30.415 | 42.026 | | | 1.00 | 52.63 | | A | C |
| ANISOU | 593 | CB | PHE A | 121 | 6929 | 4342 | 8726 | −253 | −474 | | | 1465 | A | C |
| ATOM | 594 | CG | PHE A | 121 | 48.302 | 30.553 | 41.052 | | | 1.00 | 57.08 | | A | C |
| ANISOU | 594 | CG | PHE A | 121 | 7431 | 4966 | 9293 | −358 | −449 | | | 1773 | A | C |
| ATOM | 595 | CD1 | PHE A | 121 | 48.848 | 29.442 | 40.423 | | | 1.00 | 52.86 | | A | C |
| ANISOU | 595 | CD1 | PHE A | 121 | 6803 | 4757 | 8523 | −388 | −408 | | | 1847 | A | C |
| ATOM | 596 | CD2 | PHE A | 121 | 48.862 | 31.795 | 40.797 | | | 1.00 | 61.76 | | A | C |
| ANISOU | 596 | CD2 | PHE A | 121 | 8055 | 5378 | 10031 | −423 | −428 | | | 1891 | A | C |
| ATOM | 597 | CE1 | PHE A | 121 | 49.918 | 29.579 | 39.536 | | | 1.00 | 49.64 | | A | C |
| ANISOU | 597 | CE1 | PHE A | 121 | 6320 | 4432 | 8108 | −490 | −375 | | | 2110 | A | C |
| ATOM | 598 | CE2 | PHE A | 121 | 49.921 | 31.938 | 39.915 | | | 1.00 | 55.12 | | A | C |
| ANISOU | 598 | CE2 | PHE A | 121 | 7152 | 4648 | 9143 | −529 | −377 | | | 2112 | A | O |
| ATOM | 599 | CZ | PHE A | 121 | 50.453 | 30.828 | 39.285 | | | 1.00 | 46.92 | | A | C |
| ANISOU | 599 | CZ | PHE A | 121 | 6003 | 3916 | 7908 | −561 | −356 | | | 2215 | A | C |
| ATOM | 600 | N | ALA A | 122 | 44.900 | 28.631 | 42.872 | | | 1.00 | 43.87 | | A | N |
| ANISOU | 600 | N | ALA A | 122 | 5817 | 3592 | 7260 | −42 | −416 | | | 945 | A | N |
| ATOM | 601 | CA | ALA A | 122 | 44.227 | 28.101 | 44.041 | | | 1.00 | 35.63 | | A | C |
| ANISOU | 601 | CA | ALA A | 122 | 4832 | 2555 | 6149 | −51 | −388 | | | 613 | A | C |
| ATOM | 602 | C | ALA A | 122 | 45.277 | 27.890 | 45.108 | | | 1.00 | 41.58 | | A | C |
| ANISOU | 602 | C | ALA A | 122 | 5664 | 3220 | 6916 | −216 | −406 | | | 515 | A | C |
| ATOM | 603 | O | ALA A | 122 | 46.132 | 27.008 | 44.968 | | | 1.00 | 55.99 | | A | O |
| ANISOU | 603 | O | ALA A | 122 | 7475 | 5201 | 8597 | −292 | −378 | | | 584 | A | O |
| ATOM | 604 | CB | ALA A | 122 | 43.544 | 26.788 | 43.720 | | | 1.00 | 35.19 | | A | C |
| ANISOU | 604 | CB | ALA A | 122 | 4730 | 2803 | 5837 | −10 | −294 | | | 498 | A | C |
| ATOM | 605 | N | ASN A | 123 | 45.215 | 28.699 | 46.163 | | | 1.00 | 40.73 | | A | N |
| ANISOU | 605 | N | ASN A | 123 | 5625 | 2879 | 6972 | −258 | −454 | | | 346 | A | N |
| ATOM | 606 | CA | ASN A | 123 | 46.131 | 28.564 | 47.287 | | | 1.00 | 40.16 | | A | C |
| ANISOU | 606 | CA | ASN A | 123 | 5609 | 2760 | 6889 | −403 | −487 | | | 213 | A | C |
| ATOM | 607 | C | ASN A | 123 | 45.657 | 27.373 | 48.127 | | | 1.00 | 39.49 | | A | C |
| ANISOU | 607 | C | ASN A | 123 | 5592 | 2851 | 6563 | −392 | −421 | | | −9 | A | C |
| ATOM | 608 | O | ASN A | 123 | 44.535 | 27.372 | 48.641 | | | 1.00 | 42.92 | | A | O |
| ANISOU | 608 | O | ASN A | 123 | 6065 | 3269 | 6975 | −330 | −379 | | | −218 | A | O |
| ATOM | 609 | CB | ASN A | 123 | 46.185 | 29.898 | 48.046 | | | 1.00 | 43.73 | | A | C |
| ANISOU | 609 | CB | ASN A | 123 | 6101 | 2913 | 7603 | −459 | −553 | | | 89 | A | C |
| ATOM | 610 | CG | ASN A | 123 | 46.972 | 29.839 | 49.374 | | | 1.00 | 62.73 | | A | C |
| ANISOU | 610 | CG | ASN A | 123 | 8549 | 5307 | 9979 | −604 | −600 | | | −123 | A | C |
| ATOM | 611 | OD1 | ASN A | 123 | 47.501 | 28.795 | 49.772 | | | 1.00 | 71.30 | | A | O |
| ANISOU | 611 | OD1 | ASN A | 123 | 9644 | 6607 | 10839 | −640 | −591 | | | −152 | A | O |
| ATOM | 612 | ND2 | ASN A | 123 | 47.042 | 30.985 | 50.064 | | | 1.00 | 74.71 | | A | N |
| ANISOU | 612 | ND2 | ASN A | 123 | 10085 | 6641 | 11659 | −664 | −625 | | | −271 | A | N |
| ATOM | 613 | N | ILE A | 124 | 46.483 | 26.331 | 48.205 | | | 1.00 | 36.45 | | A | N |
| ANISOU | 613 | N | ILE A | 124 | 5220 | 2636 | 5994 | −443 | −394 | | | 47 | A | N |
| ATOM | 614 | CA | ILE A | 124 | 46.132 | 25.068 | 48.845 | | | 1.00 | 32.36 | | A | C |
| ANISOU | 614 | CA | ILE A | 124 | 4793 | 2268 | 5234 | −425 | −300 | | | −99 | A | C |
| ATOM | 615 | C | ILE A | 124 | 47.245 | 24.689 | 49.815 | | | 1.00 | 54.06 | | A | C |
| ANISOU | 615 | C | ILE A | 124 | 7599 | 5058 | 7883 | −491 | −348 | | | −130 | A | C |
| ATOM | 616 | O | ILE A | 124 | 48.372 | 24.429 | 49.391 | | | 1.00 | 58.16 | | A | O |
| ANISOU | 616 | O | ILE A | 124 | 8059 | 5661 | 8380 | −515 | −386 | | | 40 | A | O |
| ATOM | 617 | CB | ILE A | 124 | 45.938 | 23.957 | 47.808 | | | 1.00 | 28.44 | | A | C |
| ANISOU | 617 | CB | ILE A | 124 | 4265 | 1959 | 4582 | −374 | −192 | | | 20 | A | C |
| ATOM | 618 | CG1 | ILE A | 124 | 44.582 | 24.114 | 47.127 | | | 1.00 | 30.24 | | A | C |
| ANISOU | 618 | CG1 | ILE A | 124 | 4436 | 2221 | 4833 | −297 | −133 | | | −41 | A | C |
| ATOM | 619 | CG2 | ILE A | 124 | 46.121 | 22.561 | 48.441 | | | 1.00 | 26.53 | | A | C |
| ANISOU | 619 | CG2 | ILE A | 124 | 4141 | 1832 | 4105 | −381 | −85 | | | −59 | A | C |
| ATOM | 62C | CD1 | ILE A | 124 | 44.224 | 22.974 | 46.224 | | | 1.00 | 30.71 | | A | C |
| ANISOU | 620 | CD1 | ILE A | 124 | 4457 | 2489 | 4721 | −269 | −9 | | | −6 | A | C |
| ATOM | 621 | N | THR A | 125 | 46.929 | 24.636 | 51.109 | | | 1.00 | 53.68 | | A | N |
| ANISOU | 621 | N | THR A | 125 | 7653 | 4986 | 7756 | −508 | −345 | | | −351 | A | N |
| ATOM | 622 | CA | THR A | 125 | 47.926 | 24.379 | 52.144 | | | 1.00 | 44.84 | | A | C |
| ANISOU | 622 | CA | THR A | 125 | 6581 | 3939 | 6518 | −548 | −412 | | | −399 | A | C |
| ATOM | 623 | C | THR A | 125 | 48.328 | 22.898 | 52.179 | | | 1.00 | 41.87 | | A | C |
| ANISOU | 623 | C | THR A | 125 | 6289 | 3736 | 5883 | −480 | −324 | | | −303 | A | C |
| ATOM | 624 | O | THR A | 125 | 47.463 | 22.007 | 52.141 | | | 1.00 | 32.26 | | A | O |
| ANISOU | 624 | O | THR A | 125 | 5176 | 2549 | 4532 | −434 | −175 | | | −347 | A | O |
| ATOM | 625 | CB | THR A | 125 | 47.379 | 24.839 | 53.500 | | | 1.00 | 37.68 | | A | C |
| ANISOU | 625 | CB | THR A | 125 | 5756 | 2973 | 5587 | −580 | −431 | | | −672 | A | C |
| ATOM | 626 | OG1 | THR A | 125 | 47.144 | 26.254 | 53.446 | | | 1.00 | 41.68 | | A | O |
| ANISOU | 626 | OG1 | THR A | 125 | 6185 | 3285 | 6366 | −636 | −510 | | | −762 | A | O |
| ATOM | 627 | CG2 | THR A | 125 | 48.358 | 24.552 | 54.612 | | | 1.00 | 45.19 | | A | C |
| ANISOU | 627 | CG2 | THR A | 125 | 6748 | 4053 | 6370 | −603 | −509 | | | −732 | A | C |
| ATOM | 628 | N | THR A | 126 | 49.629 | 22.652 | 52.273 | | | 1.00 | 37.19 | | A | N |
| ANISOU | 628 | N | THR A | 126 | 5646 | 3256 | 5229 | −473 | −408 | | | −183 | A | N |
| ATOM | 629 | CA | THR A | 126 | 50.164 | 21.301 | 52.347 | | | 1.00 | 30.10 | | A | C |
| ANISOU | 629 | CA | THR A | 126 | 4828 | 2507 | 4102 | −373 | −342 | | | −75 | A | C |
| ATOM | 630 | C | THR A | 126 | 51.186 | 21.222 | 53.478 | | | 1.00 | 35.51 | | A | C |
| ANISOU | 630 | C | THR A | 126 | 5527 | 3318 | 4646 | −343 | −458 | | | −115 | A | C |
| ATOM | 631 | O | THR A | 126 | 51.924 | 22.171 | 53.714 | | | 1.00 | 31.79 | | A | O |
| ANISOU | 631 | O | THR A | 126 | 4918 | 2870 | 4290 | −425 | −611 | | | −161 | A | O |
| ATOM | 632 | CB | THR A | 126 | 50.800 | 20.912 | 51.009 | | | 1.00 | 31.11 | | A | C |
| ANISOU | 632 | CB | THR A | 126 | 4841 | 2709 | 4271 | −341 | −319 | | | 147 | A | C |
| ATOM | 633 | CG | TYR A | 126 | 51.470 | 19.557 | 50.989 | | | 1.00 | 28.17 | | A | C |
| ANISOU | 633 | CG | TYR A | 126 | 4543 | 2469 | 3692 | −213 | −246 | | | 258 | A | C |
| ATOM | 634 | CD1 | TYR A | 126 | 50.725 | 18.393 | 50.944 | | | 1.00 | 27.33 | | A | C |
| ANISOU | 634 | CD1 | TYR A | 126 | 4598 | 2334 | 3450 | −146 | −56 | | | 252 | A | C |
| ATOM | 635 | CD2 | TYR A | 126 | 52.848 | 19.446 | 51.001 | | | 1.00 | 29.18 | | A | C |
| ANISOU | 635 | CD2 | TYR A | 126 | 4571 | 2744 | 3771 | −160 | −356 | | | 360 | A | C |
| ATOM | 636 | CE1 | TYR A | 126 | 51.334 | 17.157 | 50.921 | | | 1.00 | 27.52 | | A | C |
| ANISOU | 636 | CE1 | TYR A | 126 | 4716 | 2427 | 3312 | −17 | 33 | | | 356 | A | C |
| ATOM | 637 | CE2 | TYR A | 126 | 53.464 | 18.215 | 50.980 | | | 1.00 | 29.34 | | A | C |
| ANISOU | 637 | CE2 | TYR A | 126 | 4665 | 2872 | 3613 | −4 | −287 | | | 466 | A | C |
| ATOM | 638 | CZ | TYR A | 126 | 52.701 | 17.076 | 50.939 | | | 1.00 | 28.52 | | A | C |
| ANISOU | 638 | CZ | TYR A | 126 | 4755 | 2691 | 3392 | 73 | −86 | | | 471 | A | C |
| ATOM | 639 | OH | TYR A | 126 | 53.313 | 15.849 | 50.917 | | | 1.00 | 36.25 | | A | O |
| ANISOU | 639 | OH | TYR A | 126 | 5838 | 3723 | 4213 | 239 | 9 | | | 578 | A | O |
| ATOM | 640 | N | SER A | 127 | 51.222 | 20.095 | 54.181 | | | 1.00 | 30.67 | | A | N |
| ANISOU | 640 | N | SER A | 127 | 5081 | 2794 | 3778 | −225 | −377 | | | −98 | A | N |
| ATOM | 641 | CA | SER A | 127 | 52.180 | 19.906 | 55.259 | | | 1.00 | 32.33 | | A | C |
| ANISOU | 641 | CA | SER A | 127 | 5317 | 3175 | 3794 | −142 | −484 | | | −108 | A | C |
| ATOM | 642 | C | SER A | 127 | 53.256 | 18.969 | 54.757 | | | 1.00 | 36.73 | | A | C |
| ANISOU | 642 | C | SER A | 127 | 5824 | 3876 | 4255 | −1 | −495 | | | 102 | A | C |
| ATOM | 643 | O | SER A | 127 | 52.975 | 17.857 | 54.328 | | | 1.00 | 35.95 | | A | O |
| ANISOU | 643 | O | SER A | 127 | 5864 | 3745 | 4051 | 112 | −336 | | | 221 | A | O |
| ATOM | 644 | CB | SER A | 127 | 51.513 | 19.322 | 56.501 | | | 1.00 | 32.94 | | A | C |
| ANISOU | 644 | CB | SER A | 127 | 5634 | 3257 | 3625 | −77 | −383 | | | −205 | A | C |
| ATOM | 645 | OG | SER A | 127 | 51.055 | 20.345 | 57.364 | | | 1.00 | 35.34 | | A | O |
| ANISOU | 645 | OG | SER A | 127 | 5913 | 3597 | 3919 | −160 | −498 | | | −418 | A | O |
| ATOM | 646 | N | ILE A | 128 | 54.499 | 19.416 | 54.843 | | | 1.00 | 33.78 | | A | N |
| ANISOU | 646 | N | ILE A | 128 | 5246 | 3664 | 3927 | −13 | −673 | | | 126 | A | N |
| ATOM | 647 | CA | ILE A | 128 | 55.615 | 18.635 | 54.353 | | | 1.00 | 34.32 | | A | C |
| ANISOU | 647 | CA | ILE A | 128 | 5227 | 3906 | 3906 | 135 | −699 | | | 303 | A | C |
| ATOM | 648 | C | ILE A | 128 | 55.809 | 17.311 | 55.070 | | | 1.00 | 43.93 | | A | C |
| ANISOU | 648 | C | ILE A | 128 | 6671 | 5187 | 4832 | 370 | −608 | | | 389 | A | C |
| ATOM | 649 | O | ILE A | 128 | 55.661 | 17.214 | 56.282 | | | 1.00 | 53.83 | | A | O |
| ANISOU | 649 | O | ILE A | 128 | 8088 | 6462 | 5903 | 417 | −611 | | | 303 | A | O |
| ATOM | 650 | CB | ILE A | 128 | 56.933 | 19.418 | 54.480 | | | 1.00 | 36.23 | | A | C |
| ANISOU | 650 | CB | ILE A | 128 | 5207 | 4367 | 4192 | 83 | −914 | | | 258 | A | C |
| ATOM | 651 | CG1 | ILE A | 128 | 56.895 | 20.684 | 53.629 | | | 1.00 | 40.89 | | A | C |
| ANISOU | 651 | CG1 | ILE A | 128 | 5599 | 4845 | 5092 | −162 | −966 | | | 205 | A | C |
| ATOM | 652 | CG2 | ILE A | 128 | 58.106 | 18.546 | 54.070 | | | 1.00 | 37.08 | | A | C |
| ANISOU | 652 | CG2 | ILE A | 128 | 5207 | 4694 | 4188 | 267 | −944 | | | 424 | A | C |
| ATOM | 653 | CD1 | ILE A | 128 | 56.997 | 20.422 | 52.145 | | | 1.00 | 34.33 | | A | C |
| ANISOU | 653 | CD1 | ILE A | 128 | 4699 | 3926 | 4420 | −192 | −857 | | | 384 | A | C |
| ATOM | 654 | N | LYS A | 129 | 56.129 | 16.289 | 54.289 | | | 1.00 | 40.69 | | A | N |
| ANISOU | 654 | N | LYS A | 129 | 6290 | 4792 | 4379 | 521 | −507 | | | 564 | A | N |
| ATOM | 655 | CA | LYS A | 129 | 56.436 | 14.968 | 54.814 | | | 1.00 | 42.82 | | A | C |
| ANISOU | 655 | CA | LYS A | 129 | 6758 | 5112 | 4398 | 783 | −423 | | | 692 | A | C |
| ATOM | 656 | C | LYS A | 129 | 57.887 | 14.643 | 54.466 | | | 1.00 | 51.07 | | A | C |
| ANISOU | 656 | C | LYS A | 129 | 7605 | 6391 | 5407 | 957 | −540 | | | 820 | A | C |
| ATOM | 657 | O | LYS A | 129 | 58.565 | 15.410 | 53.776 | | | 1.00 | 47.58 | | A | O |
| ANISOU | 657 | O | LYS A | 129 | 6879 | 6062 | 5136 | 839 | −661 | | | 799 | A | O |
| ATOM | 658 | CB | LYS A | 129 | 55.464 | 13.919 | 54.250 | | | 1.00 | 46.49 | | A | C |
| ANISOU | 658 | CB | LYS A | 129 | 7467 | 5336 | 4860 | 812 | −146 | | | 755 | A | C |
| ATOM | 659 | CG | LYS A | 129 | 54.001 | 14.413 | 54.159 | | | 1.00 | 45.71 | | A | C |
| ANISOU | 659 | CG | LYS A | 129 | 7460 | 5035 | 4874 | 590 | −29 | | | 600 | A | C |
| ATOM | 660 | CD | LYS A | 129 | 53.086 | 13.407 | 53.430 | | | 1.00 | 43.43 | | A | C |
| ANISOU | 660 | CD | LYS A | 129 | 7349 | 4547 | 4603 | 580 | 248 | | | 625 | A | C |
| ATOM | 661 | CE | LYS A | 129 | 51.605 | 13.699 | 53.670 | | | 1.00 | 53.17 | | A | C |
| ANISOU | 661 | CE | LYS A | 129 | 8702 | 5627 | 5874 | 401 | 376 | | | 452 | A | C |
| ATOM | 662 | NZ | LYS A | 129 | 50.738 | 12.508 | 53.362 | | | 1.00 | 53.84 | | A | N1+ |
| ANISOU | 662 | NZ | LYS A | 129 | 9012 | 5540 | 5905 | 400 | 672 | | | 446 | A | N1+ |
| ATOM | 663 | N | GLN A | 130 | 58.382 | 13.505 | 54.958 | | | 1.00 | 57.32 | | A | N |
| ANISOU | 663 | N | GLN A | 130 | 8548 | 7258 | 5974 | 1246 | −498 | | | 956 | A | N |
| ATOM | 664 | CA | GLN A | 130 | 59.803 | 13.196 | 54.799 | | | 1.00 | 62.21 | | A | C |
| ANISOU | 664 | CA | GLN A | 130 | 8962 | 8147 | 6529 | 1457 | −632 | | | 1060 | A | C |
| ATOM | 665 | C | GLN A | 130 | 60.107 | 12.847 | 53.346 | | | 1.00 | 65.58 | | A | C |
| ANISOU | 665 | C | GLN A | 130 | 9260 | 8521 | 7137 | 1446 | −527 | | | 1142 | A | C |
| ATOM | 666 | O | GLN A | 130 | 59.453 | 11.981 | 52.757 | | | 1.00 | 67.38 | | A | O |
| ANISOU | 666 | O | GLN A | 130 | 9688 | 8521 | 7391 | 1487 | −300 | | | 1208 | A | O |
| ATOM | 667 | CB | GLN A | 130 | 60.229 | 12.048 | 55.722 | | | 1.00 | 61.32 | | A | C |
| ANISOU | 667 | CB | GLN A | 130 | 9061 | 8121 | 6118 | 1820 | −612 | | | 1207 | A | C |
| ATOM | 668 | CG | GLN A | 130 | 61.751 | 11.820 | 55.791 | | | 1.00 | 74.05 | | A | C |
| ANISOU | 668 | CG | GLN A | 130 | 10428 | 10085 | 7623 | 2081 | −798 | | | 1290 | A | C |
| ATOM | 669 | CD | GLN A | 130 | 62.553 | 13.028 | 56.327 | | | 1.00 | 82.20 | | A | C |
| ANISOU | 669 | CD | GLN A | 130 | 11117 | 11469 | 8647 | 1956 | −1090 | | | 1125 | A | C |
| ATOM | 670 | OE1 | GLN A | 130 | 62.176 | 13.651 | 57.322 | | | 1.00 | 78.95 | | A | O |
| ANISOU | 670 | OE1 | GLN A | 130 | 10752 | 11111 | 8134 | 1852 | −1187 | | | 996 | A | O |
| ATOM | 671 | NE2 | GLN A | 130 | 63.668 | 13.349 | 55.661 | | | 1.00 | 87.16 | | A | N |
| ANISOU | 671 | NE2 | GLN A | 130 | 11391 | 12344 | 9382 | 1951 | −1217 | | | 1109 | A | N |
| ATOM | 672 | N | GLY A | 131 | 61.094 | 13.526 | 52.762 | | | 1.00 | 65.61 | | A | N |
| ANISOU | 672 | N | GLY A | 131 | 8920 | 8747 | 7262 | 1371 | −681 | | | 1120 | A | N |
| ATOM | 673 | CA | GLY A | 131 | 61.418 | 13.327 | 51.362 | | | 1.00 | 55.05 | | A | C |
| ANISOU | 673 | CA | GLY A | 131 | 7428 | 7402 | 6086 | 1333 | −590 | | | 1186 | A | C |
| ATOM | 674 | C | GLY A | 131 | 60.668 | 14.220 | 50.409 | | | 1.00 | 45.28 | | A | C |
| ANISOU | 674 | C | GLY A | 131 | 6112 | 6009 | 5084 | 1014 | −532 | | | 1121 | A | C |
| ATOM | 675 | O | GLY A | 131 | 60.667 | 13.951 | 49.208 | | | 1.00 | 52.13 | | A | O |
| ANISOU | 675 | O | GLY A | 131 | 6908 | 6843 | 6057 | 978 | −416 | | | 1180 | A | O |
| ATOM | 676 | N | ASP A | 132 | 60.035 | 15.273 | 50.897 | | | 1.00 | 36.09 | | A | N |
| ANISOU | 676 | N | ASP A | 132 | 4956 | 4760 | 3997 | 797 | −609 | | | 1003 | A | N |
| ATOM | 677 | CA | ASP A | 132 | 59.287 | 16.168 | 50.039 | | | 1.00 | 40.45 | | A | C |
| ANISOU | 677 | CA | ASP A | 132 | 5447 | 5153 | 4768 | 530 | −563 | | | 961 | A | C |
| ATOM | 678 | C | ASP A | 132 | 60.136 | 17.371 | 49.661 | | | 1.00 | 40.74 | | A | C |
| ANISOU | 678 | C | ASP A | 132 | 5173 | 5335 | 4970 | 341 | −717 | | | 935 | A | C |
| ATOM | 679 | O | ASP A | 132 | 60.843 | 17.942 | 50.497 | | | 1.00 | 39.54 | | A | O |
| ANISOU | 679 | O | ASP A | 132 | 4894 | 5341 | 4789 | 313 | −885 | | | 847 | A | O |
| ATOM | 680 | CB | ASP A | 132 | 57.999 | 16.640 | 50.726 | | | 1.00 | 42.67 | | A | C |
| ANISOU | 680 | CB | ASP A | 132 | 5928 | 5220 | 5066 | 410 | −531 | | | 839 | A | C |
| ATOM | 681 | CG | ASP A | 132 | 56.831 | 15.690 | 50.510 | | | 1.00 | 49.09 | | A | C |
| ANISOU | 681 | CG | ASP A | 132 | 7006 | 5829 | 5817 | 470 | −309 | | | 852 | A | C |
| ATOM | 682 | OD1 | ASP A | 132 | 57.025 | 14.586 | 49.961 | | | 1.00 | 53.68 | | A | O |
| ANISOU | 682 | OD1 | ASP A | 132 | 7653 | 6413 | 6330 | 617 | −175 | | | 949 | A | O |
| ATOM | 683 | OD2 | ASP A | 132 | 55.705 | 16.053 | 50.888 | | | 1.00 | 51.49 | | A | O1− |
| ANISOU | 683 | OD2 | ASP A | 132 | 7445 | 5971 | 6147 | 360 | −259 | | | 744 | A | O1− |
| ATOM | 684 | N | ASN A | 133 | 60.076 | 17.736 | 48.383 | | | 1.00 | 38.91 | | A | N |
| ANISOU | 684 | N | ASN A | 133 | 4818 | 5063 | 4904 | 203 | −647 | | | 1008 | A | N |
| ATOM | 685 | CA | ASN A | 133 | 60.546 | 19.033 | 47.923 | | | 1.00 | 45.30 | | A | C |
| ANISOU | 685 | CA | ASN A | 133 | 5393 | 5909 | 5911 | −38 | −739 | | | 998 | A | C |
| ATOM | 686 | C | ASN A | 133 | 59.533 | 19.564 | 46.919 | | | 1.00 | 36.88 | | A | C |
| ANISOU | 686 | C | ASN A | 133 | 4380 | 4630 | 5002 | −191 | −629 | | | 1055 | A | C |
| ATOM | 687 | O | ASN A | 133 | 58.673 | 18.831 | 46.438 | | | 1.00 | 46.45 | | A | O |
| ANISOU | 687 | O | ASN A | 133 | 5747 | 5745 | 6156 | −106 | −490 | | | 1093 | A | O |
| ATOM | 688 | CB | ASN A | 133 | 61.940 | 18.948 | 47.298 | | | 1.00 | 50.09 | | A | C |
| ANISOU | 688 | CB | ASN A | 133 | 5718 | 6787 | 6528 | −27 | −782 | | | 1072 | A | C |
| ATOM | 689 | CG | ASN A | 133 | 61.996 | 17.959 | 46.173 | | | 1.00 | 53.97 | | A | C |
| ANISOU | 689 | CG | ASN A | 133 | 6208 | 7330 | 6967 | 102 | −631 | | | 1204 | A | C |
| ATOM | 690 | OD1 | ASN A | 133 | 61.637 | 18.279 | 45.042 | | | 1.00 | 61.80 | | A | C |
| ANISOU | 690 | OD1 | ASN A | 133 | 7156 | 8255 | 8068 | −27 | −536 | | | 1286 | A | C |
| ATOM | 691 | ND2 | ASN A | 133 | 62.425 | 16.735 | 46.476 | | | 1.00 | 47.96 | | A | N |
| ANISOU | 691 | ND2 | ASN A | 133 | 5506 | 6687 | 6032 | 371 | −602 | | | 1224 | A | N |
| ATOM | 692 | N | PHE A | 134 | 59.651 | 20.852 | 46.598 | | | 1.00 | 33.97 | | A | N |
| ANISOU | 692 | N | PHE A | 134 | 3879 | 4195 | 4832 | −415 | −687 | | | 1057 | A | N |
| ATOM | 693 | CA | PHE A | 134 | 58.726 | 21.480 | 45.661 | | | 1.00 | 35.98 | | A | C |
| ANISOU | 693 | CA | PHE A | 134 | 4179 | 4261 | 5233 | −533 | −602 | | | 1138 | A | C |
| ATOM | 694 | C | PHE A | 134 | 58.721 | 20.775 | 44.310 | | | 1.00 | 49.87 | | A | C |
| ANISOU | 694 | C | PHE A | 134 | 5890 | 6122 | 6938 | −469 | −471 | | | 1294 | A | C |
| ATOM | 695 | O | PHE A | 134 | 57.668 | 20.629 | 43.675 | | | 1.00 | 55.12 | | A | O |
| ANISOU | 695 | O | PHE A | 134 | 6660 | 6682 | 7600 | −451 | −371 | | | 1330 | A | O |
| ATOM | 696 | CB | PHE A | 134 | 59.089 | 22.952 | 45.465 | | | 1.00 | 34.56 | | A | C |
| ANISOU | 696 | CB | PHE A | 134 | 3860 | 3989 | 5281 | −771 | −669 | | | 1155 | A | C |
| ATOM | 697 | CG | PHE A | 134 | 58.857 | 23.796 | 46.673 | | | 1.00 | 39.36 | | A | C |
| ANISOU | 697 | CG | PHE A | 134 | 4528 | 4448 | 5979 | −864 | −774 | | | 969 | A | C |
| ATOM | 698 | CD1 | PHE A | 134 | 58.482 | 23.229 | 47.880 | | | 1.00 | 40.06 | | A | C |
| ANISOU | 698 | CD1 | PHE A | 134 | 4760 | 4548 | 5913 | −736 | −820 | | | 809 | A | C |
| ATOM | 699 | CD2 | PHE A | 134 | 59.009 | 25.171 | 46.601 | | | 1.00 | 52.37 | | A | C |
| ANISOU | 699 | CD2 | PHE A | 134 | 6098 | 5934 | 7866 | −1088 | −812 | | | 951 | A | C |
| ATOM | 700 | CE1 | PHE A | 134 | 58.275 | 24.018 | 48.997 | | | 1.00 | 43.69 | | A | C |
| ANISOU | 700 | CE1 | PHE A | 134 | 5262 | 4900 | 6437 | −827 | −915 | | | 614 | A | C |
| ATOM | 701 | CE2 | PHE A | 134 | 58.794 | 25.970 | 47.714 | | | 1.00 | 57.20 | | A | C |
| ANISOU | 701 | CE2 | PHE A | 134 | 6760 | 6401 | 8573 | −1184 | −898 | | | 743 | A | C |
| ATOM | 702 | CZ | PHE A | 134 | 58.417 | 25.392 | 48.913 | | | 1.00 | 48.62 | | A | C |
| ANISOU | 702 | CZ | PHE A | 134 | 5796 | 5365 | 7313 | −1052 | −955 | | | 564 | A | C |
| ATOM | 703 | N | PHE A | 135 | 59.889 | 20.360 | 43.836 | | | 1.00 | 52.63 | | A | N |
| ANISOU | 703 | N | PHE A | 135 | 6057 | 6702 | 7238 | −437 | −470 | | | 1368 | A | N |
| ATOM | 704 | CA | PHE A | 135 | 59.959 | 19.733 | 42.524 | | | 1.00 | 48.96 | | A | C |
| ANISOU | 704 | CA | PHE A | 135 | 5525 | 6360 | 6719 | −388 | −341 | | | 1495 | A | C |
| ATOM | 705 | C | PHE A | 135 | 59.148 | 18.440 | 42.481 | | | 1.00 | 39.21 | | A | C |
| ANISOU | 705 | C | PHE A | 135 | 4480 | 5088 | 5330 | −199 | −219 | | | 1447 | A | C |
| ATOM | 706 | O | PHE A | 135 | 58.219 | 18.297 | 41.669 | | | 1.00 | 32.73 | | A | O |
| ANISOU | 706 | O | PHE A | 135 | 3725 | 4209 | 4501 | −213 | −110 | | | 1475 | A | O |
| ATOM | 707 | CE | PHE A | 135 | 61.406 | 19.460 | 42.155 | | | 1.00 | 42.16 | | A | C |
| ANISOU | 707 | CB | PHE A | 135 | 4423 | 5772 | 5823 | −372 | −360 | | | 1548 | A | C |
| ATOM | 708 | CG | PHE A | 135 | 61.557 | 18.603 | 40.951 | | | 1.00 | 34.39 | | A | C |
| ANISOU | 708 | CG | PHE A | 135 | 3373 | 4945 | 4748 | −283 | −221 | | | 1638 | A | C |
| ATOM | 709 | CD1 | PHE A | 135 | 61.391 | 19.144 | 39.692 | | | 1.00 | 33.94 | | A | C |
| ANISOU | 709 | CD1 | PHE A | 135 | 3220 | 4926 | 4749 | −423 | −144 | | | 1771 | A | C |
| ATOM | 710 | CD2 | PHE A | 135 | 61.867 | 17.258 | 41.068 | | | 1.00 | 33.72 | | A | C |
| ANISOU | 710 | CD2 | PHE A | 135 | 3329 | 4971 | 4514 | −52 | −159 | | | 1589 | A | C |
| ATOM | 711 | CE1 | PHE A | 135 | 61.525 | 18.370 | 38.584 | | | 1.00 | 36.21 | | A | C |
| ANISOU | 711 | CE1 | PHE A | 135 | 3435 | 5391 | 4934 | −350 | −15 | | | 1829 | A | C |
| ATOM | 712 | CE2 | PHE A | 135 | 62.011 | 16.476 | 39.946 | | | 1.00 | 35.50 | | A | C |
| ANISOU | 712 | CE2 | PHE A | 135 | 3486 | 5336 | 4666 | 21 | −18 | | | 1638 | A | C |
| ATOM | 713 | CZ | PHE A | 135 | 61.844 | 17.034 | 38.705 | | | 1.00 | 35.81 | | A | C |
| ANISOU | 713 | CZ | PHE A | 135 | 3410 | 5446 | 4750 | −135 | 50 | | | 1745 | A | C |
| ATOM | 714 | N | ILE A | 136 | 59.480 | 17.488 | 43.363 | | | 1.00 | 34.00 | | A | N |
| ANISOU | 714 | N | ILE A | 136 | 3913 | 4465 | 4542 | −19 | −227 | | | 1370 | A | N |
| ATOM | 715 | CA | ILE A | 136 | 58.830 | 16.182 | 43.318 | | | 1.00 | 31.46 | | A | C |
| ANISOU | 715 | CA | ILE A | 136 | 3785 | 4081 | 4087 | 151 | −76 | | | 1328 | A | C |
| ATOM | 716 | C | ILE A | 136 | 57.362 | 16.292 | 43.653 | | | 1.00 | 28.76 | | A | C |
| ANISOU | 716 | C | ILE A | 136 | 3658 | 3516 | 3755 | 99 | −17 | | | 1239 | A | C |
| ATOM | 717 | O | ILE A | 136 | 56.560 | 15.488 | 43.178 | | | 1.00 | 49.27 | | A | O |
| ANISOU | 717 | O | ILE A | 136 | 6373 | 6057 | 6290 | 144 | 142 | | | 1198 | A | O |
| ATOM | 718 | CB | ILE A | 136 | 59.515 | 15.175 | 44.251 | | | 1.00 | 37.89 | | A | C |
| ANISOU | 718 | CB | ILE A | 136 | 4682 | 4952 | 4764 | 377 | −90 | | | 1298 | A | C |
| ATOM | 719 | CG1 | ILE A | 136 | 59.110 | 13.766 | 43.833 | | | 1.00 | 51.14 | | A | C |
| ANISOU | 719 | CG1 | ILE A | 136 | 6523 | 6571 | 6338 | 541 | 112 | | | 1286 | A | C |
| ATOM | 720 | CG2 | ILE A | 136 | 59.072 | 15.382 | 45.665 | | | 1.00 | 41.46 | | A | C |
| ANISOU | 720 | CG2 | ILE A | 136 | 5312 | 5272 | 5169 | 399 | −174 | | | 1212 | A | C |
| ATOM | 721 | CD1 | ILE A | 136 | 59.979 | 12.673 | 44.397 | | | 1.00 | 70.98 | | A | C |
| ANISOU | 721 | CD1 | ILE A | 136 | 9092 | 9153 | 8726 | 808 | 129 | | | 1310 | A | C |
| ATOM | 722 | N | LEU A | 137 | 56.972 | 17.295 | 44.442 | | | 1.00 | 28.75 | | A | N |
| ANISOU | 722 | N | LEU A | 137 | 3693 | 3394 | 3836 | −8 | −131 | | | 1184 | A | N |
| ATOM | 723 | CA | LEU A | 137 | 55.549 | 17.524 | 44.674 | | | 1.00 | 35.47 | | A | C |
| ANISOU | 723 | CA | LEU A | 137 | 4709 | 4056 | 4711 | −64 | −76 | | | 1088 | A | C |
| ATOM | 724 | C | LEU A | 137 | 54.862 | 17.940 | 43.384 | | | 1.00 | 33.12 | | A | C |
| ANISOU | 724 | C | LEU A | 137 | 4329 | 3764 | 4490 | −156 | −10 | | | 1147 | A | C |
| ATOM | 725 | O | LEU A | 137 | 53.840 | 17.362 | 43.001 | | | 1.00 | 32.78 | | A | O |
| ANISOU | 725 | O | LEU A | 137 | 4382 | 3686 | 4389 | −132 | 119 | | | 1078 | A | O |
| ATOM | 726 | CB | LEU A | 137 | 55.341 | 18.586 | 45.746 | | | 1.00 | 28.02 | | A | C |
| ANISOU | 726 | CB | LEU A | 137 | 3800 | 2993 | 3853 | −154 | −214 | | | 1002 | A | C |
| ATOM | 727 | CG | LEU A | 137 | 55.460 | 18.040 | 47.160 | | | 1.00 | 36.11 | | A | C |
| ANISOU | 727 | CG | LEU A | 137 | 4980 | 3999 | 4742 | −47 | −246 | | | 900 | A | C |
| ATOM | 728 | CD1 | LEU A | 137 | 55.272 | 19.174 | 48.117 | | | 1.00 | 30.01 | | A | C |
| ANISOU | 728 | CD1 | LEU A | 137 | 4208 | 3137 | 4056 | −157 | −381 | | | 787 | A | C |
| ATOM | 729 | CD2 | LEU A | 137 | 54.435 | 16.946 | 47.406 | | | 1.00 | 40.36 | | A | C |
| ANISOU | 729 | CD2 | LEU A | 137 | 5753 | 4435 | 5147 | 46 | −74 | | | 829 | A | C |
| ATOM | 730 | N | SER A | 138 | 55.429 | 18.949 | 42.706 | | | 1.00 | 33.06 | | A | N |
| ANISOU | 730 | N | SER A | 138 | 4138 | 3817 | 4606 | −265 | −93 | | | 1273 | A | N |
| ATOM | 731 | CA | SER A | 138 | 54.926 | 19.409 | 41.416 | | | 1.00 | 27.54 | | A | C |
| ANISOU | 731 | CA | SER A | 138 | 3349 | 3160 | 3955 | −330 | −42 | | | 1379 | A | C |
| ATOM | 732 | C | SER A | 138 | 54.682 | 18.254 | 40.467 | | | 1.00 | 26.93 | | A | C |
| ANISOU | 732 | C | SER A | 138 | 3263 | 3234 | 3735 | −248 | 110 | | | 1373 | A | C |
| ATOM | 733 | O | SER A | 138 | 53.594 | 18.122 | 39.897 | | | 1.00 | 42.35 | | A | O |
| ANISOU | 733 | O | SER A | 138 | 5253 | 5190 | 5649 | −247 | 190 | | | 1327 | A | O |
| ATOM | 734 | CB | SER A | 138 | 55.902 | 20.381 | 40.775 | | | 1.00 | 28.95 | | A | C |
| ANISOU | 734 | CB | SER A | 138 | 3336 | 3414 | 4248 | −447 | −115 | | | 1552 | A | C |
| ATOM | 735 | OG | SER A | 138 | 55.726 | 21.689 | 41.267 | | | 1.00 | 35.51 | | A | O |
| ANISOU | 735 | OG | SER A | 138 | 4180 | 4058 | 5255 | −562 | −218 | | | 1562 | A | O |
| ATOM | 736 | N | ILE A | 139 | 55.677 | 17.393 | 40.267 | | | 1.00 | 27.36 | | A | N |
| ANISOU | 736 | N | ILE A | 139 | 3255 | 3434 | 3707 | −173 | 157 | | | 1399 | A | N |
| ATOM | 737 | CA | ILE A | 139 | 55.515 | 16.419 | 39.187 | | | 1.00 | 33.21 | | A | C |
| ANISOU | 737 | CA | ILE A | 139 | 3958 | 4327 | 4334 | −117 | 312 | | | 1386 | A | C |
| ATOM | 738 | C | ILE A | 139 | 54.922 | 15.106 | 39.670 | | | 1.00 | 42.52 | | A | C |
| ANISOU | 738 | C | ILE A | 139 | 5330 | 5417 | 5409 | −8 | 453 | | | 1216 | A | C |
| ATOM | 739 | O | ILE A | 139 | 54.781 | 14.172 | 38.872 | | | 1.00 | 61.86 | | A | O |
| ANISOU | 739 | O | ILE A | 139 | 7768 | 7966 | 7771 | 35 | 605 | | | 1159 | A | O |
| ATOM | 740 | CB | ILE A | 139 | 56.831 | 16.142 | 38.419 | | | 1.00 | 34.80 | | A | C |
| ANISOU | 740 | CB | ILE A | 139 | 3967 | 4753 | 4504 | −92 | 328 | | | 1496 | A | C |
| ATOM | 741 | CG1 | ILE A | 139 | 57.896 | 15.498 | 39.308 | | | 1.00 | 37.89 | | A | C |
| ANISOU | 741 | CG1 | ILE A | 139 | 4375 | 5153 | 4868 | 33 | 293 | | | 1465 | A | C |
| ATOM | 742 | CG2 | ILE A | 139 | 57.365 | 17.438 | 37.795 | | | 1.00 | 29.99 | | A | C |
| ANISOU | 742 | CG2 | ILE A | 139 | 3168 | 4231 | 3994 | −238 | 231 | | | 1676 | A | C |
| ATOM | 743 | CD1 | ILE A | 139 | 58.138 | 14.032 | 38.996 | | | 1.00 | 37.21 | | A | C |
| ANISOU | 743 | CD1 | ILE A | 139 | 4336 | 5138 | 4666 | 197 | 451 | | | 1390 | A | C |
| ATOM | 744 | N | THR A | 140 | 54.542 | 14.996 | 40.938 | | | 1.00 | 36.73 | | A | N |
| ANISOU | 744 | N | THR A | 140 | 4782 | 4495 | 4678 | 27 | 427 | | | 1125 | A | N |
| ATOM | 745 | CA | THR A | 140 | 53.898 | 13.741 | 41.336 | | | 1.00 | 43.41 | | A | C |
| ANISOU | 745 | CA | THR A | 140 | 5836 | 5232 | 5426 | 108 | 600 | | | 979 | A | C |
| ATOM | 746 | C | THR A | 140 | 52.613 | 13.964 | 42.114 | | | 1.00 | 43.64 | | A | C |
| ANISOU | 746 | C | THR A | 140 | 6032 | 5086 | 5464 | 44 | 622 | | | 845 | A | C |
| ATOM | 747 | O | THR A | 140 | 51.516 | 13.659 | 41.623 | | | 1.00 | 32.52 | | A | O |
| ANISOU | 747 | O | THR A | 140 | 4655 | 3672 | 4027 | −16 | 749 | | | 721 | A | O |
| ATOM | 748 | CB | THR A | 140 | 54.808 | 12.869 | 42.192 | | | 1.00 | 42.70 | | A | C |
| ANISOU | 748 | CB | THR A | 140 | 5861 | 5094 | 5270 | 271 | 617 | | | 998 | A | C |
| ATOM | 749 | OG1 | THR A | 140 | 55.167 | 13.616 | 43.350 | | | 1.00 | 57.69 | | A | O |
| ANISOU | 749 | OG1 | THR A | 140 | 7785 | 6935 | 7197 | 274 | 443 | | | 1034 | A | O |
| ATOM | 750 | CG2 | THR A | 140 | 56.066 | 12.474 | 41.432 | | | 1.00 | 37.57 | | A | C |
| ANISOU | 750 | CG2 | THR A | 140 | 5039 | 4633 | 4605 | 363 | 620 | | | 1097 | A | C |
| ATOM | 751 | N | SER A | 141 | 52.739 | 14.451 | 43.355 | | | 1.00 | 47.89 | | A | N |
| ANISOU | 751 | N | SER A | 141 | 6665 | 5506 | 6025 | 56 | 506 | | | 844 | A | N |
| ATOM | 752 | CA | SER A | 141 | 51.568 | 14.622 | 44.210 | | | 1.00 | 42.12 | | A | C |
| ANISOU | 752 | CA | SER A | 141 | 6096 | 4619 | 5289 | 2 | 538 | | | 702 | A | C |
| ATOM | 753 | C | SER A | 141 | 50.542 | 15.510 | 43.533 | | | 1.00 | 29.10 | | A | C |
| ANISOU | 753 | C | SER A | 141 | 4338 | 2994 | 3726 | −112 | 511 | | | 653 | A | C |
| ATOM | 754 | O | SER A | 141 | 49.338 | 15.226 | 43.570 | | | 1.00 | 27.69 | | A | O |
| ANISOU | 754 | O | SER A | 141 | 4239 | 2768 | 3516 | −156 | 627 | | | 497 | A | O |
| ATOM | 755 | CB | SER A | 141 | 51.960 | 15.209 | 45.580 | | | 1.00 | 49.10 | | A | C |
| ANISOU | 755 | CB | SER A | 141 | 7054 | 5419 | 6181 | 22 | 389 | | | 707 | A | C |
| ATOM | 756 | OG | SER A | 141 | 52.416 | 14.211 | 46.493 | | | 1.00 | 46.62 | | A | C |
| ANISOU | 756 | OG | SER A | 141 | 6921 | 5057 | 5734 | 153 | 452 | | | 708 | A | C |
| ATOM | 757 | N | TYR A | 142 | 50.998 | 16.568 | 42.881 | | | 1.00 | 24.32 | | A | N |
| ANISOU | 757 | N | TYR A | 142 | 3547 | 2469 | 3224 | −154 | 370 | | | 789 | A | N |
| ATOM | 758 | CA | TYR A | 142 | 50.089 | 17.527 | 42.281 | | | 1.00 | 34.59 | | A | C |
| ANISOU | 758 | CA | TYR A | 142 | 4754 | 3780 | 4607 | −220 | 325 | | | 785 | A | C |
| ATOM | 759 | C | TYR A | 142 | 50.009 | 17.425 | 40.749 | | | 1.00 | 31.57 | | A | C |
| ANISOU | 759 | C | TYR A | 142 | 4209 | 3597 | 4189 | −225 | 379 | | | 871 | A | C |
| ATOM | 760 | O | TYR A | 142 | 49.378 | 18.283 | 40.099 | | | 1.00 | 24.66 | | A | O |
| ANISOU | 760 | O | TYR A | 142 | 3235 | 2768 | 3365 | −247 | 325 | | | 921 | A | O |
| ATOM | 761 | CB | TYR A | 142 | 50.457 | 18.923 | 42.766 | | | 1.00 | 24.79 | | A | C |
| ANISOU | 761 | CB | TYR A | 142 | 3463 | 2433 | 3522 | −267 | 143 | | | 871 | A | C |
| ATOM | 762 | CG | TYR A | 142 | 50.083 | 19.098 | 44.235 | | | 1.00 | 35.82 | | A | C |
| ANISOU | 762 | CG | TYR A | 142 | 5015 | 3661 | 4934 | −273 | 104 | | | 719 | A | C |
| ATOM | 763 | CD1 | TYR A | 142 | 48.753 | 19.173 | 44.623 | | | 1.00 | 43.35 | | A | C |
| ANISOU | 763 | CD1 | TYR A | 142 | 6055 | 4534 | 5883 | −283 | 161 | | | 549 | A | C |
| ATOM | 764 | CD2 | TYR A | 142 | 51.058 | 19.175 | 45.235 | | | 1.00 | 41.77 | | A | C |
| ANISOU | 764 | CD2 | TYR A | 142 | 5809 | 4375 | 5686 | −266 | 13 | | | 729 | A | C |
| ATOM | 765 | CE1 | TYR A | 142 | 48.394 | 19.320 | 45.939 | | | 1.00 | 46.57 | | A | C |
| ANISOU | 765 | CE1 | TYR A | 142 | 6598 | 4812 | 6286 | −293 | 140 | | | 401 | A | C |
| ATOM | 766 | CE2 | TYR A | 142 | 50.704 | 19.333 | 46.583 | | | 1.00 | 35.82 | | A | C |
| ANISOU | 766 | CE2 | TYR A | 142 | 5192 | 3503 | 4913 | −269 | −22 | | | 582 | A | C |
| ATOM | 767 | CZ | TYR A | 142 | 49.355 | 19.402 | 46.911 | | | 1.00 | 49.45 | | A | C |
| ANISOU | 767 | CZ | TYR A | 142 | 7015 | 5138 | 6636 | −287 | 50 | | | 421 | A | C |
| ATOM | 768 | OH | TYR A | 142 | 48.927 | 19.555 | 48.205 | | | 1.00 | 54.77 | | A | O |
| ANISOU | 768 | OH | TYR A | 142 | 7820 | 5715 | 7276 | −298 | 34 | | | 263 | A | O |
| ATOM | 769 | N | GLN A | 143 | 50.585 | 16.357 | 40.180 | | | 1.00 | 28.53 | | A | N |
| ANISOU | 769 | N | GLN A | 143 | 3803 | 3337 | 3701 | −187 | 493 | | | 878 | A | N |
| ATOM | 770 | CA | GLN A | 143 | 50.428 | 15.961 | 38.780 | | | 1.00 | 24.67 | | A | C |
| ANISOU | 770 | CA | GLN A | 143 | 3174 | 3070 | 3128 | −192 | 584 | | | 894 | A | C |
| ATOM | 771 | C | GLN A | 143 | 50.505 | 17.128 | 37.809 | | | 1.00 | 25.33 | | A | C |
| ANISOU | 771 | C | GLN A | 143 | 3078 | 3285 | 3262 | −223 | 471 | | | 1084 | A | C |
| ATOM | 772 | O | GLN A | 143 | 49.653 | 17.279 | 36.936 | | | 1.00 | 32.04 | | A | O |
| ANISOU | 772 | O | GLN A | 143 | 3839 | 4283 | 4050 | −226 | 500 | | | 1061 | A | O |
| ATOM | 773 | CB | GLN A | 143 | 49.116 | 15.235 | 38.548 | | | 1.00 | 30.30 | | A | C |
| ANISOU | 773 | CB | GLN A | 143 | 3936 | 3823 | 3751 | −213 | 737 | | | 668 | A | C |
| ATOM | 774 | CG | GLN A | 143 | 48.772 | 14.161 | 39.477 | | | 1.00 | 40.60 | | A | C |
| ANISOU | 774 | CG | GLN A | 143 | 5446 | 4966 | 5013 | −210 | 885 | | | 478 | A | C |
| ATOM | 775 | CD | GLN A | 143 | 47.372 | 13.672 | 39.198 | | | 1.00 | 42.17 | | A | C |
| ANISOU | 775 | CD | GLN A | 143 | 5657 | 5221 | 5146 | −277 | 1033 | | | 236 | A | C |
| ATOM | 776 | OE1 | GLN A | 143 | 46.565 | 14.408 | 38.651 | | | 1.00 | 27.39 | | A | O |
| ANISOU | 776 | OE1 | GLN A | 143 | 3648 | 3481 | 3277 | −302 | 965 | | | 214 | A | O |
| ATOM | 777 | NE2 | GLN A | 143 | 47.089 | 12.421 | 39.530 | | | 1.00 | 58.71 | | A | N |
| ANISOU | 777 | NE2 | GLN A | 143 | 7905 | 7224 | 7179 | −304 | 1245 | | | 52 | A | N |
| ATOM | 778 | N | ASN A | 144 | 51.509 | 17.981 | 37.964 | | | 1.00 | 30.12 | | A | N |
| ANISOU | 778 | N | ASN A | 144 | 3627 | 3843 | 3974 | −249 | 345 | | | 1273 | A | N |
| ATOM | 779 | CA | ASN A | 144 | 51.745 | 19.031 | 36.977 | | | 1.00 | 32.88 | | A | C |
| ANISOU | 779 | CA | ASN A | 144 | 3825 | 4298 | 4371 | −289 | 271 | | | 1493 | A | C |
| ATOM | 780 | C | ASN A | 144 | 50.541 | 19.960 | 36.860 | | | 1.00 | 31.21 | | A | C |
| ANISOU | 780 | C | ASN A | 144 | 3627 | 4017 | 4214 | −277 | 212 | | | 1502 | A | C |
| ATOM | 781 | O | ASN A | 144 | 50.310 | 20.576 | 35.816 | | | 1.00 | 28.30 | | A | O |
| ANISOU | 781 | O | ASN A | 144 | 3149 | 3783 | 3820 | −264 | 192 | | | 1664 | A | O |
| ATOM | 782 | CB | ASN A | 144 | 52.090 | 18.428 | 35.613 | | | 1.00 | 32.46 | | A | C |
| ANISOU | 782 | CB | ASN A | 144 | 3625 | 4539 | 4170 | −277 | 368 | | | 1561 | A | C |
| ATOM | 783 | CG | ASN A | 144 | 53.428 | 17.735 | 35.614 | | | 1.00 | 35.40 | | A | C |
| ANISOU | 783 | CG | ASN A | 144 | 3946 | 4990 | 4515 | −273 | 411 | | | 1589 | A | C |
| ATOM | 784 | OD1 | ASN A | 144 | 54.440 | 18.335 | 36.005 | | | 1.00 | 52.45 | | A | O |
| ANISOU | 784 | OD1 | ASN A | 144 | 6063 | 7086 | 6779 | −317 | 321 | | | 1715 | A | O |
| ATOM | 785 | ND2 | ASN A | 144 | 53.450 | 16.459 | 35.205 | | | 1.00 | 36.56 | | A | N |
| ANISOU | 785 | ND2 | ASN A | 144 | 4089 | 5274 | 4529 | −218 | 554 | | | 1451 | A | N |
| ATOM | 786 | N | LEU A | 145 | 49.726 | 20.009 | 37.926 | | | 1.00 | 34.60 | | A | N |
| ANISOU | 786 | N | LEU A | 145 | 4189 | 4255 | 4700 | −263 | 193 | | | 1324 | A | N |
| ATOM | 787 | CA | LEU A | 145 | 48.752 | 21.068 | 38.129 | | | 1.00 | 27.02 | | A | C |
| ANISOU | 787 | CA | LEU A | 145 | 3251 | 3169 | 3845 | −238 | 111 | | | 1324 | A | C |
| ATOM | 788 | C | LEU A | 145 | 49.387 | 22.328 | 38.693 | | | 1.00 | 33.66 | | A | C |
| ANISOU | 788 | C | LEU A | 145 | 4119 | 3778 | 4892 | −290 | −16 | | | 1467 | A | C |
| ATOM | 789 | O | LEU A | 145 | 48.655 | 23.303 | 38.923 | | | 1.00 | 28.64 | | A | O |
| ANISOU | 789 | O | LEU A | 145 | 3515 | 2989 | 4378 | −259 | −83 | | | 1472 | A | O |
| ATOM | 790 | CB | LEU A | 145 | 47.636 | 20.588 | 39.052 | | | 1.00 | 26.28 | | A | C |
| ANISOU | 790 | CB | LEU A | 145 | 3274 | 2985 | 3726 | −216 | 161 | | | 1047 | A | C |
| ATOM | 791 | CG | LEU A | 145 | 46.547 | 19.615 | 38.552 | | | 1.00 | 36.11 | | A | C |
| ANISOU | 791 | CG | LEU A | 145 | 4487 | 4427 | 4806 | −188 | 293 | | | 846 | A | C |
| ATOM | 792 | CD1 | LEU A | 145 | 45.920 | 18.867 | 39.739 | | | 1.00 | 24.84 | | A | C |
| ANISOU | 792 | CD1 | LEU A | 145 | 3216 | 2866 | 3355 | −220 | 387 | | | 576 | A | C |
| ATOM | 793 | CD2 | LEU A | 145 | 45.473 | 20.347 | 37.734 | | | 1.00 | 26.78 | | A | C |
| ANISOU | 793 | CD2 | LEU A | 145 | 3180 | 3386 | 3610 | −113 | 244 | | | 873 | A | C |
| ATOM | 794 | N | THR A | 146 | 50.721 | 22.303 | 38.904 | | | 1.00 | 34.60 | | A | N |
| ANISOU | 794 | N | THR A | 146 | 4215 | 3880 | 5053 | −366 | −40 | | | 1560 | A | N |
| ATOM | 795 | CA | THR A | 146 | 51.568 | 23.398 | 39.386 | | | 1.00 | 37.87 | | A | C |
| ANISOU | 795 | CA | THR A | 146 | 4623 | 4109 | 5658 | −462 | −140 | | | 1672 | A | C |
| ATOM | 796 | C | THR A | 146 | 53.002 | 23.183 | 38.904 | | | 1.00 | 48.23 | | A | C |
| ANISOU | 796 | C | THR A | 146 | 5813 | 5566 | 6945 | −538 | −128 | | | 1817 | A | C |
| ATOM | 797 | O | THR A | 146 | 53.389 | 22.074 | 38.536 | | | 1.00 | 55.27 | | A | O |
| ANISOU | 797 | O | THR A | 146 | 6658 | 6663 | 7679 | −494 | −52 | | | 1787 | A | O |
| ATOM | 798 | CB | THR A | 146 | 51.600 | 23.492 | 40.910 | | | 1.00 | 39.98 | | A | C |
| ANISOU | 798 | CB | THR A | 146 | 5006 | 4179 | 6006 | −490 | −202 | | | 1474 | A | C |
| ATOM | 799 | OG1 | THR A | 146 | 52.148 | 24.761 | 41.299 | | | 1.00 | 46.34 | | A | O |
| ANISOU | 799 | OG1 | THR A | 146 | 5797 | 4787 | 7022 | −597 | −295 | | | 1544 | A | O |
| ATOM | 800 | CG2 | THR A | 146 | 52.497 | 22.408 | 41.480 | | | 1.00 | 36.85 | | A | C |
| ANISOU | 800 | CG2 | THR A | 146 | 4618 | 3894 | 5488 | −485 | −179 | | | 1389 | A | C |
| ATOM | 801 | N | ASN A | 147 | 53.797 | 24.259 | 38.928 | | | 1.00 | 46.89 | | A | N |
| ANISOU | 801 | N | ASN A | 147 | 5590 | 5282 | 6946 | −659 | −190 | | | 1958 | A | N |
| ATOM | 802 | CA | ASN A | 147 | 55.201 | 24.240 | 38.529 | | | 1.00 | 39.37 | | A | C |
| ANISOU | 802 | CA | ASN A | 147 | 4494 | 4468 | 5997 | −763 | −179 | | | 2084 | A | C |
| ATOM | 803 | C | ASN A | 147 | 56.086 | 24.379 | 39.745 | | | 1.00 | 42.28 | | A | C |
| ANISOU | 803 | C | ASN A | 147 | 4856 | 4751 | 6457 | −847 | −260 | | | 1947 | A | C |
| ATOM | 804 | O | ASN A | 147 | 55.707 | 25.025 | 40.716 | | | 1.00 | 39.27 | | A | O |
| ANISOU | 804 | O | ASN A | 147 | 4572 | 4145 | 6205 | −882 | −333 | | | 1826 | A | O |
| ATOM | 805 | CB | ASN A | 147 | 55.554 | 25.393 | 37.593 | | | 1.00 | 34.81 | | A | C |
| ANISOU | 805 | CB | ASN A | 147 | 3840 | 3845 | 5543 | −878 | −164 | | | 2349 | A | C |
| ATOM | 806 | CG | ASN A | 147 | 55.056 | 25.182 | 36.203 | | | 1.00 | 59.27 | | A | C |
| ANISOU | 806 | CG | ASN A | 147 | 6887 | 7139 | 8493 | −797 | −83 | | | 2530 | A | C |
| ATOM | 807 | OD1 | ASN A | 147 | 54.845 | 24.047 | 35.776 | | | 1.00 | 69.48 | | A | O |
| ANISOU | 807 | OD1 | ASN A | 147 | 8143 | 8671 | 9584 | −697 | −24 | | | 2452 | A | O |
| ATOM | 808 | ND2 | ASN A | 147 | 54.856 | 26.273 | 35.476 | | | 1.00 | 57.94 | | A | N |
| ANISOU | 808 | ND2 | ASN A | 147 | 6727 | 6884 | 8403 | −820 | −75 | | | 2726 | A | N |
| ATOM | 809 | N | TYR A | 148 | 57.293 | 23.816 | 39.666 | | | 1.00 | 47.38 | | A | N |
| ANISOU | 809 | N | TYR A | 148 | 5367 | 5602 | 7033 | −877 | −252 | | | 1958 | A | N |
| ATOM | 810 | CA | TYR A | 148 | 58.303 | 24.136 | 40.679 | | | 1.00 | 44.51 | | A | C |
| ANISOU | 810 | CA | TYR A | 148 | 4938 | 5217 | 6758 | −976 | −345 | | | 1851 | A | C |
| ATOM | 811 | C | TYR A | 148 | 58.563 | 25.642 | 40.751 | | | 1.00 | 43.93 | | A | C |
| ANISOU | 811 | C | TYR A | 148 | 4830 | 4933 | 6929 | −1186 | −385 | | | 1916 | A | C |
| ATOM | 812 | O | TYR A | 148 | 58.730 | 26.206 | 41.838 | | | 1.00 | 47.71 | | A | O |
| ANISOU | 812 | O | TYR A | 148 | 5336 | 5267 | 7525 | −1269 | −472 | | | 1756 | A | O |
| ATOM | 813 | CB | TYR A | 148 | 59.612 | 23.393 | 40.407 | | | 1.00 | 39.84 | | A | C |
| ANISOU | 813 | CB | TYR A | 148 | 4160 | 4917 | 6059 | −966 | −328 | | | 1872 | A | C |
| ATOM | 814 | CG | TYR A | 148 | 60.802 | 24.075 | 41.040 | | | 1.00 | 55.21 | | A | C |
| ANISOU | 814 | CG | TYR A | 148 | 5954 | 6898 | 8127 | −1130 | −415 | | | 1813 | A | C |
| ATOM | 815 | CD1 | TYR A | 148 | 61.104 | 23.900 | 42.387 | | | 1.00 | 51.22 | | A | C |
| ANISOU | 815 | CD1 | TYR A | 148 | 5467 | 6396 | 7598 | −1091 | −529 | | | 1606 | A | C |
| ATOM | 816 | CD2 | TYR A | 148 | 61.618 | 24.904 | 40.283 | | | 1.00 | 67.24 | | A | C |
| ANISOU | 816 | CD2 | TYR A | 148 | 7304 | 8469 | 9774 | −1336 | −376 | | | 1957 | A | C |
| ATOM | 817 | CE1 | TYR A | 148 | 62.178 | 24.524 | 42.953 | | | 1.00 | 59.53 | | A | C |
| ANISOU | 817 | CE1 | TYR A | 148 | 6348 | 7524 | 8745 | −1250 | −615 | | | 1516 | A | C |
| ATOM | 818 | CE2 | TYR A | 148 | 62.703 | 25.544 | 40.843 | | | 1.00 | 72.64 | | A | C |
| ANISOU | 818 | CE2 | TYR A | 148 | 7823 | 9199 | 10577 | −1522 | −440 | | | 1869 | A | C |
| ATOM | 819 | CZ | TYR A | 148 | 62.984 | 25.349 | 42.183 | | | 1.00 | 72.46 | | A | C |
| ANISOU | 819 | CZ | TYR A | 148 | 7798 | 9206 | 10527 | −1478 | −568 | | | 1633 | A | C |
| ATOM | 820 | OH | TYR A | 148 | 64.074 | 25.978 | 42.762 | | | 1.00 | 86.28 | | A | O |
| ANISOU | 820 | OH | TYR A | 148 | 9352 | 11049 | 12381 | −1670 | −642 | | | 1505 | A | O |
| ATOM | 821 | N | LEU A | 149 | 58.562 | 26.314 | 39.605 | | | 1.00 | 42.74 | | A | N |
| ANISOU | 821 | N | LEU A | 149 | 4633 | 4754 | 6854 | −1273 | −310 | | | 2149 | A | N |
| ATOM | 822 | CA | LEU A | 149 | 58.855 | 27.742 | 39.597 | | | 1.00 | 49.24 | | A | C |
| ANISOU | 822 | CA | LEU A | 149 | 5446 | 5336 | 7928 | −1484 | −312 | | | 2238 | A | C |
| ATOM | 823 | C | LEU A | 149 | 57.769 | 28.536 | 40.329 | | | 1.00 | 53.65 | | A | C |
| ANISOU | 823 | C | LEU A | 149 | 6192 | 5548 | 8645 | −1457 | −363 | | | 2139 | A | C |
| ATOM | 824 | O | LEU A | 149 | 58.072 | 29.458 | 41.102 | | | 1.00 | 42.06 | | A | O |
| ANISOU | 824 | O | LEU A | 149 | 4737 | 3859 | 7383 | −1615 | −408 | | | 2028 | A | O |
| ATOM | 825 | CB | LEU A | 149 | 59.029 | 28.201 | 38.146 | | | 1.00 | 50.97 | | A | C |
| ANISOU | 825 | CB | LEU A | 149 | 5607 | 5610 | 8150 | −1544 | −199 | | | 2537 | A | C |
| ATOM | 826 | CG | LEU A | 149 | 59.166 | 29.692 | 37.904 | | | 1.00 | 52.16 | | A | C |
| ANISOU | 826 | CG | LEU A | 149 | 5824 | 5512 | 8481 | −1667 | −154 | | | 2590 | A | C |
| ATOM | 827 | CD1 | LEU A | 149 | 60.488 | 30.158 | 38.438 | | | 1.00 | 59.93 | | A | C |
| ANISOU | 827 | CD1 | LEU A | 149 | 6664 | 6494 | 9612 | −1927 | −158 | | | 2502 | A | C |
| ATOM | 828 | CD2 | LEU A | 149 | 59.056 | 29.955 | 36.441 | | | 1.00 | 46.09 | | A | C |
| ANISOU | 828 | CD2 | LEU A | 149 | 5062 | 4849 | 7599 | −1605 | −46 | | | 2822 | A | C |
| ATOM | 829 | N | GLU A | 150 | 56.519 | 28.231 | 39.996 | | | 1.00 | 62.03 | | A | N |
| ANISOU | 829 | N | GLU A | 150 | 7385 | 6574 | 9609 | −1262 | −353 | | | 2143 | A | N |
| ATOM | 830 | CA | GLU A | 150 | 55.352 | 28.859 | 40.604 | | | 1.00 | 60.71 | | A | C |
| ANISOU | 830 | CA | GLU A | 150 | 7380 | 6108 | 9579 | −1205 | −394 | | | 2041 | A | C |
| ATOM | 831 | C | GLU A | 150 | 55.251 | 28.513 | 42.079 | | | 1.00 | 57.93 | | A | C |
| ANISOU | 831 | C | GLU A | 150 | 7089 | 5719 | 9203 | −1182 | −478 | | | 1723 | A | C |
| ATOM | 832 | O | GLU A | 150 | 54.910 | 29.352 | 42.907 | | | 1.00 | 64.26 | | A | O |
| ANISOU | 832 | O | GLU A | 150 | 7965 | 6265 | 10184 | −1250 | −527 | | | 1579 | A | O |
| ATOM | 833 | CB | GLU A | 150 | 54.081 | 28.446 | 39.868 | | | 1.00 | 48.37 | | A | C |
| ANISOU | 833 | CB | GLU A | 150 | 5894 | 4586 | 7899 | −1003 | −354 | | | 2137 | A | C |
| ATOM | 834 | CG | GLU A | 150 | 54.035 | 28.944 | 38.438 | | | 1.00 | 45.40 | | A | C |
| ANISOU | 834 | CG | GLU A | 150 | 5465 | 4333 | 7452 | −964 | −273 | | | 2385 | A | C |
| ATOM | 835 | CD | GLU A | 150 | 54.145 | 30.449 | 38.359 | | | 1.00 | 52.10 | | A | C |
| ANISOU | 835 | CD | GLU A | 150 | 6384 | 4924 | 8486 | −1017 | −252 | | | 2462 | A | C |
| ATOM | 836 | OE1 | GLU A | 150 | 53.542 | 31.137 | 39.204 | | | 1.00 | 56.22 | | A | O |
| ANISOU | 836 | OE1 | GLU A | 150 | 6888 | 5508 | 8966 | −999 | −182 | | | 2686 | A | O |
| ATOM | 837 | OE2 | GLU A | 150 | 54.834 | 30.944 | 37.446 | | | 1.00 | 46.34 | | A | O1− |
| ANISOU | 837 | OE2 | GLU A | 150 | 5740 | 3927 | 7940 | −1076 | −295 | | | 2292 | A | O1− |
| ATOM | 838 | N | PHE A | 151 | 55.554 | 27.258 | 42.395 | | | 1.00 | 57.95 | | A | N |
| ANISOU | 838 | N | PHE A | 151 | 7073 | 5969 | 8976 | −1076 | −482 | | | 1616 | A | N |
| ATOM | 839 | CA | PHE A | 151 | 55.521 | 26.764 | 43.763 | | | 1.00 | 34.94 | | A | C |
| ANISOU | 839 | CA | PHE A | 151 | 4205 | 3080 | 5991 | −1054 | −555 | | | 1357 | A | C |
| ATOM | 840 | C | PHE A | 151 | 56.549 | 27.520 | 44.588 | | | 1.00 | 43.35 | | A | C |
| ANISOU | 840 | C | PHE A | 151 | 5178 | 4087 | 7207 | −1243 | −637 | | | 1255 | A | C |
| ATOM | 841 | O | PHE A | 151 | 56.288 | 27.912 | 45.721 | | | 1.00 | 37.61 | | A | O |
| ANISOU | 841 | O | PHE A | 151 | 4516 | 3230 | 6545 | −1281 | −707 | | | 1036 | A | O |
| ATOM | 842 | CB | PHE A | 151 | 55.867 | 25.279 | 43.763 | | | 1.00 | 33.35 | | A | C |
| ANISOU | 842 | CB | PHE A | 151 | 3974 | 3162 | 5535 | −925 | −525 | | | 1341 | A | C |
| ATOM | 843 | CG | PHE A | 151 | 55.324 | 24.524 | 44.935 | | | 1.00 | 41.41 | | A | C |
| ANISOU | 843 | CG | PHE A | 151 | 5134 | 4190 | 6409 | −800 | −541 | | | 1137 | A | C |
| ATOM | 844 | CD1 | PHE A | 151 | 55.211 | 25.123 | 46.171 | | | 1.00 | 51.49 | | A | C |
| ANISOU | 844 | CD1 | PHE A | 151 | 6490 | 5320 | 7752 | −843 | −619 | | | 942 | A | C |
| ATOM | 845 | CD2 | PHE A | 151 | 54.950 | 23.202 | 44.801 | | | 1.00 | 39.40 | | A | C |
| ANISOU | 845 | CD2 | PHE A | 151 | 4939 | 4088 | 5944 | −648 | −460 | | | 1135 | A | C |
| ATOM | 846 | CE1 | PHE A | 151 | 54.717 | 24.422 | 47.251 | | | 1.00 | 51.23 | | A | C |
| ANISOU | 846 | CE1 | PHE A | 151 | 6595 | 5314 | 7556 | −732 | −617 | | | 773 | A | C |
| ATOM | 847 | CE2 | PHE A | 151 | 54.460 | 22.493 | 45.874 | | | 1.00 | 29.52 | | A | C |
| ANISOU | 847 | CE2 | PHE A | 151 | 3840 | 2824 | 4551 | −544 | −445 | | | 971 | A | C |
| ATOM | 848 | CZ | PHE A | 151 | 54.342 | 23.105 | 47.102 | | | 1.00 | 36.55 | | A | C |
| ANISOU | 848 | CZ | PHE A | 151 | 4809 | 3591 | 5489 | −584 | −525 | | | 803 | A | C |
| ATOM | 849 | N | LYS A | 152 | 57.728 | 27.710 | 44.007 | | | 1.00 | 47.68 | | A | N |
| ANISOU | 849 | N | LYS A | 152 | 5555 | 4754 | 7805 | −1378 | −621 | | | 1388 | A | N |
| ATOM | 850 | CA | LYS A | 152 | 58.804 | 28.440 | 44.652 | | | 1.00 | 56.30 | | A | C |
| ANISOU | 850 | CA | LYS A | 152 | 6520 | 5840 | 9033 | −1586 | −689 | | | 1258 | A | C |
| ATOM | 851 | C | LYS A | 152 | 58.452 | 29.905 | 44.873 | | | 1.00 | 58.92 | | A | C |
| ANISOU | 851 | C | LYS A | 152 | 6923 | 5805 | 9660 | −1759 | −679 | | | 1224 | A | C |
| ATOM | 852 | O | LYS A | 152 | 58.750 | 30.461 | 45.923 | | | 1.00 | 66.57 | | A | O |
| ANISOU | 852 | O | LYS A | 152 | 7880 | 6677 | 10736 | −1882 | −752 | | | 984 | A | O |
| ATOM | 853 | CB | LYS A | 152 | 60.086 | 28.326 | 43.831 | | | 1.00 | 57.30 | | A | C |
| ANISOU | 853 | CB | LYS A | 152 | 6423 | 6212 | 9137 | −1700 | −655 | | | 1394 | A | C |
| ATOM | 854 | CG | LYS A | 152 | 61.316 | 28.891 | 44.516 | | | 1.00 | 60.53 | | A | C |
| ANISOU | 854 | CG | LYS A | 152 | 6650 | 6670 | 9679 | −1942 | −715 | | | 1241 | A | C |
| ATOM | 855 | CD | LYS A | 152 | 62.563 | 28.646 | 43.684 | | | 1.00 | 64.48 | | A | C |
| ANISOU | 855 | CD | LYS A | 152 | 6902 | 7471 | 10127 | −2037 | −671 | | | 1362 | A | C |
| ATOM | 856 | CE | LYS A | 152 | 62.520 | 29.408 | 42.369 | | | 1.00 | 61.10 | | A | C |
| ANISOU | 856 | CE | LYS A | 152 | 6463 | 6940 | 9813 | −2151 | −522 | | | 1666 | A | C |
| ATOM | 857 | NZ | LYS A | 152 | 62.619 | 30.878 | 42.572 | | | 1.00 | 58.31 | | A | N1+ |
| ANISOU | 857 | NZ | LYS A | 152 | 6069 | 6333 | 9755 | −2470 | −462 | | | 1688 | A | N1+ |
| ATOM | 858 | N | ASN A | 153 | 57.811 | 30.531 | 43.890 | | | 1.00 | 50.89 | | A | N |
| ANISOU | 858 | N | ASN A | 153 | 5987 | 4580 | 8769 | −1753 | −589 | | | 1456 | A | N |
| ATOM | 859 | CA | ASN A | 153 | 57.464 | 31.940 | 44.022 | | | 1.00 | 48.84 | | A | C |
| ANISOU | 859 | CA | ASN A | 153 | 5828 | 3964 | 8766 | −1860 | −550 | | | 1422 | A | C |
| ATOM | 860 | C | ASN A | 153 | 56.334 | 32.161 | 45.016 | | | 1.00 | 45.60 | | A | C |
| ANISOU | 860 | C | ASN A | 153 | 5581 | 3355 | 8391 | −1742 | −604 | | | 1192 | A | C |
| ATOM | 861 | O | ASN A | 153 | 56.202 | 33.267 | 45.550 | | | 1.00 | 51.62 | | A | O |
| ANISOU | 861 | O | ASN A | 153 | 6417 | 3885 | 9313 | −1818 | −580 | | | 1034 | A | O |
| ATOM | 862 | CB | ASN A | 153 | 57.088 | 32.535 | 42.666 | | | 1.00 | 60.23 | | A | C |
| ANISOU | 862 | CB | ASN A | 153 | 7344 | 5336 | 10204 | −1777 | −418 | | | 1693 | A | C |
| ATOM | 863 | CG | ASN A | 153 | 58.221 | 32.450 | 41.638 | | | 1.00 | 81.82 | | A | C |
| ANISOU | 863 | CG | ASN A | 153 | 9926 | 8268 | 12895 | −1907 | −340 | | | 1907 | A | C |
| ATOM | 864 | OD1 | ASN A | 153 | 59.391 | 32.704 | 41.947 | | | 1.00 | 80.12 | | A | O |
| ANISOU | 864 | OD1 | ASN A | 153 | 9568 | 8107 | 12767 | −2137 | −339 | | | 1826 | A | O |
| ATOM | 865 | ND2 | ASN A | 153 | 57.870 | 32.086 | 40.407 | | | 1.00 | 84.62 | | A | N |
| ANISOU | 865 | ND2 | ASN A | 153 | 10292 | 8760 | 13100 | −1763 | −272 | | | 2156 | A | N |
| ATOM | 866 | N | PHE A | 154 | 55.574 | 31.114 | 45.298 | | | 1.00 | 45.23 | | A | N |
| ANISOU | 866 | N | PHE A | 154 | 5598 | 3420 | 8167 | −1550 | −649 | | | 1147 | A | N |
| ATOM | 867 | CA | PHE A | 154 | 54.470 | 31.184 | 46.243 | | | 1.00 | 54.36 | | A | C |
| ANISOU | 867 | CA | PHE A | 154 | 6895 | 4433 | 9327 | −1441 | −690 | | | 910 | A | C |
| ATOM | 868 | C | PHE A | 154 | 54.917 | 31.074 | 47.703 | | | 1.00 | 52.99 | | A | C |
| ANISOU | 868 | C | PHE A | 154 | 6693 | 4336 | 9104 | −1526 | −783 | | | 588 | A | C |
| ATOM | 869 | O | PHE A | 154 | 54.163 | 31.376 | 48.619 | | | 1.00 | 46.78 | | A | O |
| ANISOU | 869 | O | PHE A | 154 | 6008 | 3395 | 8371 | −1500 | −814 | | | 356 | A | O |
| ATOM | 870 | CB | PHE A | 154 | 53.462 | 30.088 | 45.916 | | | 1.00 | 51.51 | | A | C |
| ANISOU | 870 | CB | PHE A | 154 | 6608 | 4237 | 8726 | −1202 | −661 | | | 949 | A | C |
| ATOM | 871 | CG | PHE A | 154 | 52.039 | 30.547 | 45.936 | | | 1.00 | 58.75 | | A | C |
| ANISOU | 871 | CG | PHE A | 154 | 7652 | 4942 | 9727 | −1066 | −637 | | | 911 | A | C |
| ATOM | 872 | CD1 | PHE A | 154 | 51.089 | 29.858 | 46.658 | | | 1.00 | 63.32 | | A | C |
| ANISOU | 872 | CD1 | PHE A | 154 | 8267 | 5336 | 10457 | −1038 | −579 | | | 1026 | A | C |
| ATOM | 873 | CD2 | PHE A | 154 | 51.653 | 31.664 | 45.229 | | | 1.00 | 58.55 | | A | C |
| ANISOU | 873 | CD2 | PHE A | 154 | 7708 | 5006 | 9531 | −920 | −635 | | | 728 | A | C |
| ATOM | 874 | CE1 | PHE A | 154 | 49.778 | 30.275 | 46.674 | | | 1.00 | 64.91 | | A | C |
| ANISOU | 874 | CE1 | PHE A | 154 | 8562 | 5410 | 10690 | −867 | −557 | | | 977 | A | C |
| ATOM | 875 | CE2 | PHE A | 154 | 50.344 | 32.088 | 45.242 | | | 1.00 | 58.93 | | A | C |
| ANISOU | 875 | CE2 | PHE A | 154 | 7839 | 4904 | 9647 | −789 | −612 | | | 668 | A | C |
| ATOM | 876 | CZ | PHE A | 154 | 49.405 | 31.394 | 45.968 | | | 1.00 | 61.90 | | A | C |
| ANISOU | 876 | CZ | PHE A | 154 | 8230 | 5085 | 10205 | −750 | −584 | | | 792 | A | C |
| ATOM | 877 | N | ASN A | 155 | 56.150 | 30.634 | 47.907 | | | 1.00 | 56.07 | | A | N |
| ANISOU | 877 | N | ASN A | 155 | 6936 | 4984 | 9383 | −1618 | −830 | | | 560 | A | N |
| ATOM | 878 | CA | ASN A | 155 | 56.707 | 30.463 | 49.244 | | | 1.00 | 42.99 | | A | C |
| ANISOU | 878 | CA | ASN A | 155 | 5227 | 3477 | 7631 | −1674 | −935 | | | 269 | A | C |
| ATOM | 879 | C | ASN A | 155 | 58.071 | 31.121 | 49.302 | | | 1.00 | 53.14 | | A | C |
| ANISOU | 879 | C | ASN A | 155 | 6315 | 4824 | 9053 | −1926 | −971 | | | 215 | A | C |
| ATOM | 880 | O | ASN A | 155 | 59.078 | 30.474 | 49.607 | | | 1.00 | 55.45 | | A | O |
| ANISOU | 880 | O | ASN A | 155 | 6450 | 5445 | 9172 | −1934 | −1041 | | | 163 | A | O |
| ATOM | 881 | CB | ASN A | 155 | 56.806 | 28.983 | 49.583 | | | 1.00 | 40.81 | | A | C |
| ANISOU | 881 | CB | ASN A | 155 | 4957 | 3536 | 7012 | −1477 | −969 | | | 264 | A | C |
| ATOM | 882 | CG | ASN A | 155 | 55.471 | 28.344 | 49.652 | | | 1.00 | 47.59 | | A | C |
| ANISOU | 882 | CG | ASN A | 155 | 6004 | 4333 | 7743 | −1276 | −913 | | | 265 | A | C |
| ATOM | 883 | OD1 | ASN A | 155 | 54.914 | 28.192 | 50.736 | | | 1.00 | 50.43 | | A | O |
| ANISOU | 883 | OD1 | ASN A | 155 | 6468 | 4685 | 8007 | −1219 | −948 | | | 51 | A | O |
| ATOM | 884 | ND2 | ASN A | 155 | 54.915 | 28.003 | 48.494 | | | 1.00 | 37.01 | | A | N |
| ANISOU | 884 | ND2 | ASN A | 155 | 4701 | 2964 | 6395 | −1180 | −820 | | | 491 | A | N |
| ATOM | 885 | N | PRO A | 156 | 58.141 | 32.416 | 49.011 | | | 1.00 | 54.66 | | A | N |
| ANISOU | 885 | N | PRO A | 156 | 6504 | 4700 | 9563 | −2134 | −917 | | | 221 | A | N |
| ATOM | 886 | CA | PRO A | 156 | 59.457 | 33.049 | 48.818 | | | 1.00 | 67.76 | | A | C |
| ANISOU | 886 | CA | PRO A | 156 | 7962 | 6406 | 11376 | −2416 | −907 | | | 204 | A | C |
| ATOM | 887 | C | PRO A | 156 | 60.333 | 33.035 | 50.063 | | | 1.00 | 80.29 | | A | C |
| ANISOU | 887 | C | PRO A | 156 | 9388 | 8234 | 12885 | −2544 | −1032 | | | −143 | A | C |
| ATOM | 888 | O | PRO A | 156 | 61.564 | 33.003 | 49.949 | | | 1.00 | 89.55 | | A | O |
| ANISOU | 888 | O | PRO A | 156 | 10330 | 9651 | 14042 | −2705 | −1056 | | | −168 | A | O |
| ATOM | 889 | CB | PRO A | 156 | 59.091 | 34.475 | 48.388 | | | 1.00 | 64.42 | | A | C |
| ANISOU | 889 | CB | PRO A | 156 | 7673 | 5600 | 11203 | −2522 | −756 | | | 245 | A | C |
| ATOM | 890 | CG | PRO A | 156 | 57.728 | 34.708 | 48.978 | | | 1.00 | 58.74 | | A | C |
| ANISOU | 890 | CG | PRO A | 156 | 7173 | 4673 | 10473 | −2332 | −756 | | | 118 | A | C |
| ATOM | 891 | CD | PRO A | 156 | 57.031 | 33.384 | 48.935 | | | 1.00 | 52.19 | | A | C |
| ANISOU | 891 | CD | PRO A | 156 | 6379 | 3996 | 9454 | −2103 | −840 | | | 213 | A | C |
| ATOM | 892 | N | ASN A | 157 | 59.742 | 33.049 | 51.261 | | | 1.00 | 79.61 | | A | N |
| ANISOU | 892 | N | ASN A | 157 | 9396 | 8125 | 12727 | −2474 | −1114 | | | −426 | A | N |
| ATOM | 893 | CA | ASN A | 157 | 60.574 | 33.012 | 52.458 | | | 1.00 | 88.43 | | A | C |
| ANISOU | 893 | CA | ASN A | 157 | 10346 | 9528 | 13724 | −2577 | −1245 | | | −760 | A | C |
| ATOM | 894 | C | ASN A | 157 | 61.174 | 31.625 | 52.663 | | | 1.00 | 86.80 | | A | C |
| ANISOU | 894 | C | ASN A | 157 | 10032 | 9807 | 13141 | −2366 | −1344 | | | −700 | A | C |
| ATOM | 895 | O | ASN A | 157 | 62.312 | 31.493 | 53.138 | | | 1.00 | 92.75 | | A | O |
| ANISOU | 895 | O | ASN A | 157 | 10552 | 10899 | 13789 | −2455 | −1444 | | | −854 | A | O |
| ATOM | 896 | CB | ASN A | 157 | 59.764 | 33.466 | 53.680 | | | 1.00 | 95.73 | | A | C |
| ANISOU | 896 | CB | ASN A | 157 | 11403 | 10304 | 14665 | −2566 | −1296 | | | −1085 | A | C |
| ATOM | 897 | CG | ASN A | 157 | 60.570 | 33.393 | 55.010 | | | 1.00 | 105.12 | | A | C |
| ANISOU | 897 | CG | ASN A | 157 | 12417 | 11849 | 15675 | −2653 | −1448 | | | −1456 | A | C |
| ATOM | 898 | OD1 | ASN A | 157 | 60.221 | 32.631 | 55.924 | | | 1.00 | 103.15 | | A | O |
| ANISOU | 898 | OD1 | ASN A | 157 | 12233 | 11836 | 15124 | −2451 | −1540 | | | −1573 | A | O |
| ATOM | 899 | ND2 | ASN A | 157 | 61.647 | 34.193 | 55.112 | | | 1.00 | 108.12 | | A | N |
| ANISOU | 899 | ND2 | ASN A | 157 | 12568 | 12281 | 16231 | −2959 | −1467 | | | −1643 | A | N |
| ATOM | 900 | N | LEU A | 158 | 60.399 | 30.602 | 52.321 | | | 1.00 | 84.78 | | A | N |
| ANISOU | 900 | N | LEU A | 158 | 9934 | 9594 | 12684 | −2083 | −1310 | | | −484 | A | N |
| ATOM | 901 | CA | LEU A | 158 | 60.802 | 29.205 | 52.471 | | | 1.00 | 77.27 | | A | C |
| ANISOU | 901 | CA | LEU A | 158 | 8937 | 9041 | 11381 | −1848 | −1385 | | | −437 | A | C |
| ATOM | 902 | C | LEU A | 158 | 61.840 | 28.750 | 51.459 | | | 1.00 | 65.84 | | A | C |
| ANISOU | 902 | C | LEU A | 158 | 7292 | 7821 | 9904 | −1854 | −1360 | | | −219 | A | C |
| ATOM | 903 | O | LEU A | 158 | 62.032 | 29.379 | 50.425 | | | 1.00 | 69.25 | | A | O |
| ANISOU | 903 | O | LEU A | 158 | 7591 | 8152 | 10571 | −2081 | −1297 | | | −142 | A | O |
| ATOM | 904 | CB | LEU A | 158 | 59.584 | 28.286 | 52.410 | | | 1.00 | 76.99 | | A | C |
| ANISOU | 904 | CB | LEU A | 158 | 9160 | 8934 | 11160 | −1574 | −1331 | | | −327 | A | C |
| ATOM | 905 | CG | LEU A | 158 | 58.614 | 28.415 | 53.579 | | | 1.00 | 90.51 | | A | C |
| ANISOU | 905 | CG | LEU A | 158 | 11040 | 10455 | 12896 | −1583 | −1350 | | | −571 | A | C |
| ATOM | 906 | CD1 | LEU A | 158 | 57.597 | 27.286 | 53.540 | | | 1.00 | 92.23 | | A | C |
| ANISOU | 906 | CD1 | LEU A | 158 | 11503 | 10589 | 12951 | −1351 | −1270 | | | −486 | A | C |
| ATOM | 907 | CD2 | LEU A | 158 | 59.383 | 28.398 | 54.891 | | | 1.00 | 96.84 | | A | C |
| ANISOU | 907 | CD2 | LEU A | 158 | 11735 | 11524 | 13535 | −1626 | −1497 | | | −874 | A | C |
| ATOM | 908 | N | SER A | 159 | 62.519 | 27.656 | 51.778 | | | 1.00 | 59.54 | | A | N |
| ANISOU | 908 | N | SER A | 159 | 6479 | 7328 | 8814 | −1599 | −1397 | | | −121 | A | N |
| ATOM | 909 | CA | SER A | 159 | 63.544 | 27.113 | 50.903 | | | 1.00 | 56.08 | | A | C |
| ANISOU | 909 | CA | SER A | 159 | 5841 | 7161 | 8308 | −1556 | −1383 | | | 48 | A | C |
| ATOM | 910 | C | SER A | 159 | 63.128 | 25.783 | 50.300 | | | 1.00 | 57.78 | | A | C |
| ANISOU | 910 | C | SER A | 159 | 6212 | 7394 | 8350 | −1277 | −1291 | | | 292 | A | C |
| ATOM | 911 | O | SER A | 159 | 62.741 | 24.869 | 51.017 | | | 1.00 | 59.75 | | A | O |
| ANISOU | 911 | O | SER A | 159 | 6672 | 7616 | 8415 | −1063 | −1292 | | | 274 | A | O |
| ATOM | 912 | CB | SER A | 159 | 64.832 | 26.902 | 51.691 | | | 1.00 | 60.87 | | A | C |
| ANISOU | 912 | CB | SER A | 159 | 6204 | 8204 | 8719 | −1507 | −1539 | | | −125 | A | C |
| ATOM | 913 | OG | SER A | 159 | 64.659 | 25.875 | 52.652 | | | 1.00 | 60.11 | | A | O |
| ANISOU | 913 | OG | SER A | 159 | 6252 | 8250 | 8336 | −1242 | −1627 | | | −208 | A | O |
| ATOM | 914 | N | PRO A | 160 | 63.200 | 25.678 | 48.970 | | | 1.00 | 51.58 | | A | N |
| ANISOU | 914 | N | PRO A | 160 | 5328 | 6654 | 7618 | −1291 | −1195 | | | 508 | A | N |
| ATOM | 915 | CA | PRO A | 160 | 62.878 | 24.451 | 48.242 | | | 1.00 | 44.60 | | A | C |
| ANISOU | 915 | CA | PRO A | 160 | 4537 | 5864 | 6545 | −1027 | −1112 | | | 694 | A | C |
| ATOM | 916 | C | PRO A | 160 | 63.967 | 23.401 | 48.420 | | | 1.00 | 58.71 | | A | C |
| ANISOU | 916 | C | PRO A | 160 | 6196 | 8020 | 8090 | −810 | −1198 | | | 658 | A | C |
| ATOM | 917 | O | PRO A | 160 | 65.112 | 23.777 | 48.673 | | | 1.00 | 69.53 | | A | O |
| ANISOU | 917 | O | PRO A | 160 | 7422 | 9596 | 9402 | −839 | −1341 | | | 479 | A | O |
| ATOM | 918 | CB | PRO A | 160 | 62.837 | 24.914 | 46.791 | | | 1.00 | 49.03 | | A | C |
| ANISOU | 918 | CB | PRO A | 160 | 4997 | 6382 | 7250 | −1149 | −988 | | | 905 | A | C |
| ATOM | 919 | CG | PRO A | 160 | 63.771 | 26.067 | 46.750 | | | 1.00 | 52.48 | | A | C |
| ANISOU | 919 | CG | PRO A | 160 | 5374 | 6599 | 7968 | −1464 | −984 | | | 866 | A | C |
| ATOM | 920 | CD | PRO A | 160 | 63.601 | 26.766 | 48.065 | | | 1.00 | 45.65 | | A | C |
| ANISOU | 920 | CD | PRO A | 160 | 4415 | 5813 | 7117 | −1567 | −1126 | | | 596 | A | C |
| ATOM | 921 | N | THR A | 161 | 63.565 | 22.128 | 48.391 | | | 1.00 | 59.31 | | A | N |
| ANISOU | 921 | N | THR A | 161 | 6321 | 8192 | 8021 | −579 | −1111 | | | 816 | A | N |
| ATOM | 922 | CA | THR A | 161 | 64.388 | 20.896 | 48.471 | | | 1.00 | 54.15 | | A | C |
| ANISOU | 922 | CA | THR A | 161 | 5546 | 7872 | 7156 | −328 | −1165 | | | 829 | A | C |
| ATOM | 923 | C | THR A | 161 | 64.794 | 20.464 | 49.875 | | | 1.00 | 52.62 | | A | C |
| ANISOU | 923 | C | THR A | 161 | 5466 | 7790 | 6739 | −110 | −1285 | | | 710 | A | C |
| ATOM | 924 | O | THR A | 161 | 65.286 | 19.356 | 50.067 | | | 1.00 | 46.22 | | A | O |
| ANISOU | 924 | O | THR A | 161 | 4778 | 7059 | 5725 | 195 | −1251 | | | 793 | A | O |
| ATOM | 925 | CB | THR A | 161 | 65.613 | 20.840 | 47.511 | | | 1.00 | 54.54 | | A | C |
| ANISOU | 925 | CB | THR A | 161 | 5219 | 8231 | 7272 | −461 | −1235 | | | 794 | A | C |
| ATOM | 926 | OG1 | THR A | 161 | 66.657 | 21.696 | 47.993 | | | 1.00 | 62.79 | | A | O |
| ANISOU | 926 | OG1 | THR A | 161 | 6115 | 9338 | 8403 | −683 | −1373 | | | 588 | A | O |
| ATOM | 927 | CG2 | THR A | 161 | 65.223 | 21.254 | 46.104 | | | 1.00 | 45.47 | | A | C |
| ANISOU | 927 | CG2 | THR A | 161 | 3962 | 7011 | 6304 | −658 | −1096 | | | 944 | A | C |
| ATOM | 928 | N | LEU A | 162 | 64.562 | 21.330 | 50.851 | | | 1.00 | 52.04 | | A | N |
| ANISOU | 928 | N | LEU A | 162 | 5354 | 7728 | 6692 | −257 | −1418 | | | 516 | A | N |
| ATOM | 929 | CA | LEU A | 162 | 64.866 | 21.022 | 52.239 | | | 1.00 | 64.26 | | A | C |
| ANISOU | 929 | CA | LEU A | 162 | 6994 | 9420 | 8000 | −69 | −1543 | | | 392 | A | C |
| ATOM | 930 | C | LEU A | 162 | 63.957 | 21.827 | 53.142 | | | 1.00 | 63.20 | | A | C |
| ANISOU | 930 | C | LEU A | 162 | 6996 | 9064 | 7952 | −267 | −1578 | | | 213 | A | C |
| ATOM | 931 | O | LEU A | 162 | 64.129 | 23.034 | 53.279 | | | 1.00 | 64.43 | | A | O |
| ANISOU | 931 | O | LEU A | 162 | 6990 | 9167 | 8322 | −563 | −1625 | | | 71 | A | O |
| ATOM | 932 | CB | LEU A | 162 | 66.328 | 21.331 | 52.566 | | | 1.00 | 77.78 | | A | C |
| ANISOU | 932 | CB | LEU A | 162 | 8390 | 11584 | 9581 | −4 | −1729 | | | 271 | A | C |
| ATOM | 933 | CG | LEU A | 162 | 67.361 | 20.219 | 52.371 | | | 1.00 | 76.33 | | A | C |
| ANISOU | 933 | CG | LEU A | 162 | 8017 | 11739 | 9247 | 265 | −1755 | | | 388 | A | C |
| ATOM | 934 | CD1 | LEU A | 162 | 68.765 | 20.748 | 52.612 | | | 1.00 | 72.25 | | A | C |
| ANISOU | 934 | CD1 | LEU A | 162 | 7085 | 11662 | 8704 | 177 | −1941 | | | 202 | A | C |
| ATOM | 935 | CD2 | LEU A | 162 | 67.061 | 19.048 | 53.291 | | | 1.00 | 76.28 | | A | C |
| ANISOU | 935 | CD2 | LEU A | 162 | 8261 | 11778 | 8944 | 680 | −1752 | | | 500 | A | C |
| ATOM | 936 | N | LEU A | 163 | 62.992 | 21.167 | 53.761 | | | 1.00 | 61.17 | | A | N |
| ANISOU | 936 | N | LEU A | 163 | 7034 | 8675 | 7532 | −107 | −1542 | | | 208 | A | N |
| ATOM | 937 | CA | LEU A | 163 | 62.094 | 21.860 | 54.661 | | | 1.00 | 52.53 | | A | C |
| ANISOU | 937 | CA | LEU A | 163 | 6079 | 7399 | 6481 | −254 | −1569 | | | 18 | A | C |
| ATOM | 938 | C | LEU A | 163 | 61.801 | 20.948 | 55.832 | | | 1.00 | 45.80 | | A | C |
| ANISOU | 938 | C | LEU A | 163 | 5472 | 6613 | 5317 | 0 | −1577 | | | 6 | A | C |
| ATOM | 939 | O | LEU A | 163 | 61.715 | 19.737 | 55.664 | | | 1.00 | 45.72 | | A | O |
| ANISOU | 939 | O | LEU A | 163 | 5659 | 6547 | 5166 | 232 | −1456 | | | 195 | A | O |
| ATOM | 940 | CB | LEU A | 163 | 60.812 | 22.251 | 53.932 | | | 1.00 | 54.19 | | A | C |
| ANISOU | 940 | CB | LEU A | 163 | 6439 | 7205 | 6948 | −430 | −1419 | | | 63 | A | C |
| ATOM | 941 | CG | LEU A | 163 | 60.172 | 23.566 | 54.371 | | | 1.00 | 64.23 | | A | C |
| ANISOU | 941 | CG | LEU A | 163 | 7739 | 8253 | 8412 | −675 | −1448 | | | −151 | A | C |
| ATOM | 942 | CD1 | LEU A | 163 | 61.241 | 24.610 | 54.646 | | | 1.00 | 70.98 | | A | C |
| ANISOU | 942 | CD1 | LEU A | 163 | 8314 | 9251 | 9404 | −908 | −1584 | | | −343 | A | C |
| ATOM | 943 | CD2 | LEU A | 163 | 59.198 | 24.059 | 53.315 | | | 1.00 | 62.23 | | A | C |
| ANISOU | 943 | CD2 | LEU A | 163 | 7596 | 7638 | 8411 | −795 | −1307 | | | −58 | A | C |
| ATOM | 944 | N | PRO A | 164 | 61.634 | 21.520 | 57.028 | | | 1.00 | 46.59 | | A | N |
| ANISOU | 944 | N | PRO A | 164 | 5574 | 6829 | 5298 | −44 | −1699 | | | −213 | A | N |
| ATOM | 945 | CA | PRO A | 164 | 61.360 | 20.616 | 58.159 | | | 1.00 | 60.42 | | A | C |
| ANISOU | 945 | CA | PRO A | 164 | 7540 | 8718 | 6698 | 220 | −1716 | | | −198 | A | C |
| ATOM | 946 | C | PRO A | 164 | 60.024 | 19.900 | 58.023 | | | 1.00 | 61.61 | | A | C |
| ANISOU | 946 | C | PRO A | 164 | 8047 | 8552 | 6811 | 310 | −1508 | | | −70 | A | C |
| ATOM | 947 | O | PRO A | 164 | 59.100 | 20.357 | 57.345 | | | 1.00 | 54.48 | | A | O |
| ANISOU | 947 | O | PRO A | 164 | 7222 | 7333 | 6143 | 135 | −1384 | | | −80 | A | O |
| ATOM | 948 | CB | PRO A | 164 | 61.367 | 21.539 | 59.392 | | | 1.00 | 49.61 | | A | C |
| ANISOU | 948 | CB | PRO A | 164 | 6098 | 7493 | 5259 | 78 | −1866 | | | −500 | A | C |
| ATOM | 949 | CG | PRO A | 164 | 61.987 | 22.786 | 58.938 | | | 1.00 | 53.51 | | A | C |
| ANISOU | 949 | CG | PRO A | 164 | 6290 | 7992 | 6050 | −227 | −1951 | | | −673 | A | C |
| ATOM | 950 | CD | PRO A | 164 | 61.711 | 22.925 | 57.466 | | | 1.00 | 48.12 | | A | C |
| ANISOU | 950 | CD | PRO A | 164 | 5608 | 7001 | 5675 | −343 | −1801 | | | −490 | A | C |
| ATOM | 951 | N | LEU A | 165 | 59.945 | 18.750 | 58.683 | | | 1.00 | 64.65 | | A | N |
| ANISOU | 951 | N | LEU A | 165 | 8644 | 9036 | 6885 | 592 | −1465 | | | 53 | A | N |
| ATOM | 952 | CA | LEU A | 165 | 58.727 | 17.958 | 58.677 | | | 1.00 | 48.19 | | A | C |
| ANISOU | 952 | CA | LEU A | 165 | 6905 | 6674 | 4732 | 671 | −1247 | | | 159 | A | C |
| ATOM | 953 | C | LEU A | 165 | 57.590 | 18.754 | 59.291 | | | 1.00 | 58.93 | | A | C |
| ANISOU | 953 | C | LEU A | 165 | 8379 | 7861 | 6149 | 467 | −1211 | | | −53 | A | C |
| ATOM | 954 | O | LEU A | 165 | 57.779 | 19.464 | 60.284 | | | 1.00 | 76.17 | | A | O |
| ANISOU | 954 | O | LEU A | 165 | 10483 | 10215 | 8245 | 396 | −1356 | | | −258 | A | O |
| ATOM | 955 | CB | LEU A | 165 | 58.969 | 16.678 | 59.454 | | | 1.00 | 48.92 | | A | C |
| ANISOU | 955 | CB | LEU A | 165 | 7209 | 6913 | 4466 | 1003 | −1213 | | | 324 | A | C |
| ATOM | 956 | CG | LEU A | 165 | 57.953 | 15.562 | 59.415 | | | 1.00 | 47.98 | | A | C |
| ANISOU | 956 | CG | LEU A | 165 | 7458 | 6526 | 4245 | 1123 | −954 | | | 478 | A | C |
| ATOM | 957 | CD1 | LEU A | 165 | 57.933 | 14.995 | 58.007 | | | 1.00 | 42.01 | | A | C |
| ANISOU | 957 | CD1 | LEU A | 165 | 6694 | 5576 | 3694 | 1134 | −802 | | | 632 | A | C |
| ATOM | 958 | CD2 | LEU A | 165 | 58.321 | 14.498 | 60.460 | | | 1.00 | 48.24 | | A | C |
| ANISOU | 958 | CD2 | LEU A | 165 | 7703 | 6731 | 3895 | 1454 | −952 | | | 633 | A | C |
| ATOM | 959 | N | ASP A | 166 | 56.409 | 18.641 | 58.686 | | | 1.00 | 54.54 | | A | N |
| ANISOU | 959 | N | ASP A | 166 | 7993 | 6989 | 5741 | 376 | −1016 | | | −28 | A | N |
| ATOM | 960 | CA | ASP A | 166 | 55.166 | 19.290 | 59.105 | | | 1.00 | 57.07 | | A | C |
| ANISOU | 960 | CA | ASP A | 166 | 8430 | 7116 | 6138 | 206 | −943 | | | −219 | A | C |
| ATOM | 961 | C | ASP A | 166 | 55.162 | 20.786 | 58.824 | | | 1.00 | 47.93 | | A | C |
| ANISOU | 961 | C | ASP A | 166 | 7052 | 5877 | 5283 | −46 | −1055 | | | −416 | A | C |
| ATOM | 962 | O | ASP A | 166 | 54.197 | 21.467 | 59.193 | | | 1.00 | 53.48 | | A | O |
| ANISOU | 962 | O | ASP A | 166 | 7820 | 6424 | 6075 | −179 | −1014 | | | −603 | A | O |
| ATOM | 963 | CB | ASP A | 166 | 54.850 | 19.033 | 60.588 | | | 1.00 | 61.25 | | A | C |
| ANISOU | 963 | CB | ASP A | 166 | 9139 | 7781 | 6354 | 290 | −952 | | | −327 | A | C |
| ATOM | 964 | CG | ASP A | 166 | 54.318 | 17.619 | 60.839 | | | 1.00 | 74.83 | | A | C |
| ANISOU | 964 | CG | ASP A | 166 | 11174 | 9436 | 7822 | 487 | −748 | | | −139 | A | C |
| ATOM | 965 | OD1 | ASP A | 166 | 54.185 | 16.831 | 59.881 | | | 1.00 | 84.51 | | A | O |
| ANISOU | 965 | OD1 | ASP A | 166 | 12476 | 10506 | 9129 | 551 | −598 | | | 44 | A | O |
| ATOM | 966 | OD2 | ASP A | 166 | 54.019 | 17.285 | 62.001 | | | 1.00 | 81.83 | | A | O1− |
| ANISOU | 966 | OD2 | ASP A | 166 | 12243 | 10423 | 8426 | 569 | −720 | | | −179 | A | O1− |
| ATOM | 967 | N | THR A | 167 | 56.194 | 21.323 | 58.185 | | | 1.00 | 44.25 | | A | N |
| ANISOU | 967 | N | THR A | 167 | 6332 | 5498 | 4982 | −118 | −1178 | | | −384 | A | N |
| ATOM | 968 | CA | THR A | 167 | 56.174 | 22.717 | 57.782 | | | 1.00 | 47.51 | | A | C |
| ANISOU | 968 | CA | THR A | 167 | 6567 | 5769 | 5717 | −368 | −1243 | | | −534 | A | C |
| ATOM | 969 | C | THR A | 167 | 55.063 | 22.940 | 56.773 | | | 1.00 | 49.52 | | A | C |
| ANISOU | 969 | C | THR A | 167 | 6909 | 5700 | 6205 | −445 | −1087 | | | −466 | A | C |
| ATOM | 970 | O | THR A | 167 | 54.859 | 22.141 | 55.852 | | | 1.00 | 39.00 | | A | O |
| ANISOU | 970 | O | THR A | 167 | 5638 | 4311 | 4868 | −352 | −968 | | | −261 | A | O |
| ATOM | 971 | CB | THR A | 167 | 57.522 | 23.121 | 57.190 | | | 1.00 | 49.58 | | A | C |
| ANISOU | 971 | CB | THR A | 167 | 6548 | 6187 | 6103 | −440 | −1366 | | | −481 | A | C |
| ATOM | 972 | OG1 | THR A | 167 | 58.476 | 23.214 | 58.251 | | | 1.00 | 56.55 | | A | C |
| ANISOU | 972 | OG1 | THR A | 167 | 7298 | 7395 | 6792 | −410 | −1539 | | | −629 | A | C |
| ATOM | 973 | CG2 | THR A | 167 | 57.428 | 24.464 | 56.498 | | | 1.00 | 55.13 | | A | C |
| ANISOU | 973 | CG2 | THR A | 167 | 7111 | 6662 | 7174 | −702 | −1372 | | | −563 | A | C |
| ATOM | 974 | N | LYS A | 168 | 54.308 | 24.008 | 56.979 | | | 1.00 | 51.92 | | A | N |
| ANISOU | 974 | N | LYS A | 168 | 6625 | 6988 | 6112 | −2440 | −1048 | | | −799 | A | N |
| ATOM | 975 | CA | LYS A | 168 | 53.161 | 24.279 | 56.137 | | | 1.00 | 42.13 | | A | C |
| ANISOU | 975 | CA | LYS A | 168 | 5368 | 5684 | 4956 | −2199 | −873 | | | −907 | A | C |
| ATOM | 976 | C | LYS A | 168 | 53.621 | 25.152 | 54.980 | | | 1.00 | 39.36 | | A | C |
| ANISOU | 976 | C | LYS A | 168 | 4950 | 5136 | 4869 | −2069 | −869 | | | −801 | A | C |
| ATOM | 977 | O | LYS A | 168 | 54.306 | 26.158 | 55.193 | | | 1.00 | 40.91 | | A | O |
| ANISOU | 977 | O | LYS A | 168 | 5194 | 5185 | 5164 | −2173 | −951 | | | −823 | A | O |
| ATOM | 978 | CB | LYS A | 168 | 52.027 | 24.931 | 56.941 | | | 1.00 | 48.14 | | A | C |
| ANISOU | 978 | CB | LYS A | 168 | 6255 | 6441 | 5595 | −2206 | −751 | | | −1219 | A | C |
| ATOM | 979 | CG | LYS A | 168 | 51.295 | 23.958 | 57.901 | | | 1.00 | 54.24 | | A | C |
| ANISOU | 979 | CG | LYS A | 168 | 7061 | 7453 | 6096 | −2290 | −713 | | | −1304 | A | C |
| ATOM | 980 | CD | LYS A | 168 | 50.040 | 24.589 | 58.523 | | | 1.00 | 62.22 | | A | C |
| ANISOU | 980 | CD | LYS A | 168 | 8165 | 8474 | 7003 | −2237 | −537 | | | −1596 | A | C |
| ATOM | 981 | CE | LYS A | 168 | 50.352 | 25.792 | 59.424 | | | 1.00 | 71.52 | | A | C |
| ANISOU | 981 | CE | LYS A | 168 | 9524 | 9515 | 8134 | −2379 | −540 | | | −1839 | A | C |
| ATOM | 982 | NZ | LYS A | 168 | 49.103 | 26.391 | 59.996 | | | 1.00 | 78.54 | | A | N1+ |
| ANISOU | 982 | NZ | LYS A | 168 | 10506 | 10402 | 8934 | −2286 | −321 | | | −2133 | A | N1+ |
| ATOM | 983 | N | VAL A | 169 | 53.294 | 24.712 | 53.758 | | | 1.00 | 42.82 | | A | N |
| ANISOU | 983 | N | VAL A | 169 | 5282 | 5582 | 5407 | −1866 | −790 | | | −668 | A | N |
| ATOM | 984 | CA | VAL A | 169 | 53.573 | 25.411 | 52.509 | | | 1.00 | 42.56 | | A | C |
| ANISOU | 984 | CA | VAL A | 169 | 5165 | 5404 | 5600 | −1724 | −766 | | | −541 | A | C |
| ATOM | 985 | C | VAL A | 169 | 52.251 | 25.660 | 51.776 | | | 1.00 | 46.30 | | A | C |
| ANISOU | 985 | C | VAL A | 169 | 5616 | 5855 | 6120 | −1532 | −622 | | | −628 | A | C |
| ATOM | 986 | O | VAL A | 169 | 51.217 | 25.050 | 52.075 | | | 1.00 | 36.20 | | A | O |
| ANISOU | 986 | O | VAL A | 169 | 4358 | 4696 | 4699 | −1497 | −544 | | | −733 | A | O |
| ATOM | 987 | CB | VAL A | 169 | 54.553 | 24.630 | 51.595 | | | 1.00 | 41.33 | | A | C |
| ANISOU | 987 | CB | VAL A | 169 | 4894 | 5299 | 5510 | −1670 | −807 | | | −255 | A | C |
| ATOM | 988 | CG1 | VAL A | 169 | 55.944 | 24.474 | 52.265 | | | 1.00 | 43.32 | | A | C |
| ANISOU | 988 | CG1 | VAL A | 169 | 5121 | 5571 | 5767 | −1852 | −957 | | | −106 | A | C |
| ATOM | 989 | CG2 | VAL A | 169 | 53.963 | 23.278 | 51.189 | | | 1.00 | 34.39 | | A | C |
| ANISOU | 989 | CG2 | VAL A | 169 | 4000 | 4565 | 4503 | −1570 | −733 | | | −212 | A | C |
| ATOM | 990 | N | SER A | 170 | 52.321 | 26.559 | 50.779 | | | 1.00 | 41.45 | | A | N |
| ANISOU | 990 | N | SER A | 170 | 4938 | 5096 | 5716 | −1420 | −600 | | | −545 | A | N |
| ATOM | 991 | CA | SER A | 170 | 51.197 | 26.972 | 49.942 | | | 1.00 | 36.83 | | A | C |
| ANISOU | 991 | CA | SER A | 170 | 4296 | 4472 | 5224 | −1246 | −487 | | | −563 | A | C |
| ATOM | 992 | C | SER A | 170 | 51.411 | 26.462 | 48.522 | | | 1.00 | 38.80 | | A | C |
| ANISOU | 992 | C | SER A | 170 | 4442 | 4782 | 5519 | −1138 | −481 | | | −327 | A | C |
| ATOM | 993 | O | SER A | 170 | 52.249 | 26.987 | 47.775 | | | 1.00 | 42.38 | | A | O |
| ANISOU | 993 | O | SER A | 170 | 4831 | 5149 | 6122 | −1119 | −526 | | | −163 | A | O |
| ATOM | 994 | CB | SER A | 170 | 51.057 | 28.489 | 49.960 | | | 1.00 | 39.58 | | A | C |
| ANISOU | 994 | CB | SER A | 170 | 4658 | 4594 | 5788 | −1214 | −468 | | | −650 | A | C |
| ATOM | 995 | OG | SER A | 170 | 50.625 | 28.917 | 51.237 | | | 1.00 | 54.95 | | A | O |
| ANISOU | 995 | OG | SER A | 170 | 6732 | 6486 | 7660 | −1290 | −429 | | | −914 | A | O |
| ATOM | 996 | N | VAL A | 171 | 50.670 | 25.426 | 48.159 | | | 1.00 | 42.14 | | A | N |
| ANISOU | 996 | N | VAL A | 171 | 4857 | 5357 | 5797 | −1082 | −429 | | | −308 | A | N |
| ATOM | 997 | CA | VAL A | 171 | 50.707 | 24.895 | 46.804 | | | 1.00 | 39.99 | | A | C |
| ANISOU | 997 | CA | VAL A | 171 | 4527 | 5147 | 5520 | −990 | −409 | | | −122 | A | C |
| ATOM | 998 | C | VAL A | 171 | 49.867 | 25.831 | 45.945 | | | 1.00 | 45.35 | | A | C |
| ANISOU | 998 | C | VAL A | 171 | 5125 | 5761 | 6343 | −884 | −362 | | | −79 | A | C |
| ATOM | 999 | O | VAL A | 171 | 48.725 | 26.143 | 46.322 | | | 1.00 | 44.59 | | A | O |
| ANISOU | 999 | O | VAL A | 171 | 5018 | 5667 | 6256 | −849 | −309 | | | −195 | A | O |
| ATOM | 1000 | CB | VAL A | 171 | 50.195 | 23.449 | 46.745 | | | 1.00 | 31.91 | | A | C |
| ANISOU | 1000 | CB | VAL A | 171 | 3555 | 4286 | 4284 | −998 | −389 | | | −126 | A | C |
| ATOM | 1001 | CG1 | VAL A | 171 | 50.404 | 22.878 | 45.366 | | | 1.00 | 42.09 | | A | C |
| ANISOU | 1001 | CG1 | VAL A | 171 | 4832 | 5621 | 5541 | −925 | −367 | | | 42 | A | C |
| ATOM | 1002 | CG2 | VAL A | 171 | 50.892 | 22.585 | 47.776 | | | 1.00 | 31.65 | | A | C |
| ANISOU | 1002 | CG2 | VAL A | 171 | 3587 | 4304 | 4134 | −1108 | −440 | | | −167 | A | C |
| ATOM | 1003 | N | PRO A | 172 | 50.412 | 26.366 | 44.826 | | | 1.00 | 40.47 | | A | N |
| ANISOU | 1003 | N | PRO A | 172 | 4432 | 5089 | 5858 | −829 | −376 | | | 105 | A | N |
| ATOM | 1004 | CA | PRO A | 172 | 49.580 | 27.149 | 43.904 | | | 1.00 | 46.71 | | A | C |
| ANISOU | 1004 | CA | PRO A | 172 | 5125 | 5842 | 6779 | −737 | −346 | | | 195 | A | C |
| ATOM | 1005 | C | PRO A | 172 | 49.100 | 26.298 | 42.741 | | | 1.00 | 45.57 | | A | C |
| ANISOU | 1005 | C | PRO A | 172 | 4966 | 5861 | 6489 | −702 | −326 | | | 330 | A | C |
| ATOM | 1006 | O | PRO A | 172 | 49.918 | 25.785 | 41.976 | | | 1.00 | 50.60 | | A | O |
| ANISOU | 1006 | O | PRO A | 172 | 5620 | 6556 | 7051 | −704 | −332 | | | 457 | A | O |
| ATOM | 1007 | CB | PRO A | 172 | 50.530 | 28.256 | 43.431 | | | 1.00 | 41.01 | | A | C |
| ANISOU | 1007 | CB | PRO A | 172 | 4333 | 4972 | 6279 | −730 | −392 | | | 333 | A | C |
| ATOM | 1008 | CG | PRO A | 172 | 51.842 | 27.586 | 43.415 | | | 1.00 | 38.19 | | A | C |
| ANISOU | 1008 | CG | PRO A | 172 | 4007 | 4669 | 5833 | −788 | −428 | | | 415 | A | C |
| ATOM | 1009 | CD | PRO A | 172 | 51.841 | 26.524 | 44.511 | | | 1.00 | 37.99 | | A | C |
| ANISOU | 1009 | CD | PRO A | 172 | 4090 | 4726 | 5617 | −859 | −427 | | | 250 | A | C |
| ATOM | 1010 | N | LEU A | 173 | 47.793 | 26.123 | 42.591 | | | 1.00 | 36.58 | | A | N |
| ANISOU | 1010 | N | LEU A | 173 | 3799 | 4802 | 5297 | −678 | −300 | | | 309 | A | N |
| ATOM | 1011 | CA | LEU A | 173 | 47.268 | 25.366 | 41.465 | | | 1.00 | 39.04 | | A | C |
| ANISOU | 1011 | CA | LEU A | 173 | 4114 | 5267 | 5454 | −683 | −307 | | | 441 | A | C |
| ATOM | 1012 | C | LEU A | 173 | 46.806 | 26.314 | 40.361 | | | 1.00 | 41.87 | | A | C |
| ANISOU | 1012 | C | LEU A | 173 | 4336 | 5611 | 5962 | −632 | −320 | | | 634 | A | C |
| ATOM | 1013 | O | LEU A | 173 | 46.165 | 27.329 | 40.633 | | | 1.00 | 53.85 | | A | O |
| ANISOU | 1013 | O | LEU A | 173 | 5742 | 7031 | 7688 | −575 | −307 | | | 636 | A | O |
| ATOM | 1014 | CB | LEU A | 173 | 46.122 | 24.460 | 41.916 | | | 1.00 | 41.92 | | A | C |
| ANISOU | 1014 | CB | LEU A | 173 | 4521 | 5758 | 5647 | −727 | −302 | | | 349 | A | C |
| ATOM | 1015 | CG | LEU A | 173 | 46.455 | 23.409 | 42.978 | | | 1.00 | 38.39 | | A | C |
| ANISOU | 1015 | CG | LEU A | 173 | 4200 | 5348 | 5039 | −794 | −302 | | | 188 | A | C |
| ATOM | 1016 | CD1 | LEU A | 173 | 45.385 | 22.355 | 43.027 | | | 1.00 | 42.98 | | A | C |
| ANISOU | 1016 | CD1 | LEU A | 173 | 4824 | 6077 | 5428 | −857 | −320 | | | 168 | A | C |
| ATOM | 1017 | CD2 | LEU A | 173 | 47.789 | 22.761 | 42.741 | | | 1.00 | 34.05 | | A | C |
| ANISOU | 1017 | CD2 | LEU A | 173 | 3745 | 4779 | 4414 | −812 | −312 | | | 218 | A | C |
| ATOM | 1018 | N | PHE A | 174 | 47.184 | 26.020 | 39.125 | | | 1.00 | 36.56 | | A | N |
| ANISOU | 1018 | N | PHE A | 174 | 3674 | 5032 | 5186 | −650 | −339 | | | 802 | A | N |
| ATOM | 1019 | CA | PHE A | 174 | 46.785 | 26.850 | 37.999 | | | 1.00 | 47.05 | | A | C |
| ANISOU | 1019 | CA | PHE A | 174 | 4869 | 6390 | 6615 | −631 | −369 | | | 1023 | A | C |
| ATOM | 1020 | C | PHE A | 174 | 45.283 | 26.750 | 37.899 | | | 1.00 | 60.55 | | A | C |
| ANISOU | 1020 | C | PHE A | 174 | 6506 | 8197 | 8302 | −648 | −394 | | | 1067 | A | C |
| ATOM | 1021 | O | PHE A | 174 | 44.771 | 25.655 | 37.957 | | | 1.00 | 57.31 | | A | O |
| ANISOU | 1021 | O | PHE A | 174 | 6191 | 7935 | 7650 | −726 | −413 | | | 1035 | A | O |
| ATOM | 1022 | CB | PHE A | 174 | 47.376 | 26.312 | 36.700 | | | 1.00 | 41.11 | | A | C |
| ANISOU | 1022 | CB | PHE A | 174 | 4174 | 5766 | 5679 | −671 | −374 | | | 1179 | A | C |
| ATOM | 1023 | CG | PHE A | 174 | 48.848 | 26.520 | 36.567 | | | 1.00 | 42.57 | | A | C |
| ANISOU | 1023 | CG | PHE A | 174 | 4369 | 5883 | 5923 | −639 | −342 | | | 1224 | A | C |
| ATOM | 1024 | CD1 | PHE A | 174 | 49.357 | 27.772 | 36.312 | | | 1.00 | 42.26 | | A | C |
| ANISOU | 1024 | CD1 | PHE A | 174 | 4184 | 5724 | 6147 | −601 | −368 | | | 1361 | A | C |
| ATOM | 1025 | CD2 | PHE A | 174 | 49.717 | 25.456 | 36.675 | | | 1.00 | 37.78 | | A | C |
| ANISOU | 1025 | CD2 | PHE A | 174 | 3905 | 5329 | 5122 | −646 | −285 | | | 1157 | A | C |
| ATOM | 1026 | CE1 | PHE A | 174 | 50.712 | 27.964 | 36.182 | | | 1.00 | 42.70 | | A | C |
| ANISOU | 1026 | CE1 | PHE A | 174 | 4226 | 5738 | 6261 | −587 | −350 | | | 1441 | A | C |
| ATOM | 1027 | CE2 | PHE A | 174 | 51.070 | 25.639 | 36.547 | | | 1.00 | 35.11 | | A | C |
| ANISOU | 1027 | CE2 | PHE A | 174 | 3540 | 4949 | 4850 | −608 | −246 | | | 1240 | A | C |
| ATOM | 1028 | CZ | PHE A | 174 | 51.571 | 26.895 | 36.301 | | | 1.00 | 37.24 | | A | C |
| ANISOU | 1028 | CZ | PHE A | 174 | 3653 | 5123 | 5372 | −588 | −285 | | | 1388 | A | C |
| ATOM | 1029 | N | CYS A | 175 | 44.588 | 27.879 | 37.793 | | | 1.00 | 61.71 | | A | N |
| ANISOU | 1029 | N | CYS A | 175 | 6479 | 8255 | 8714 | −578 | −395 | | | 1160 | A | N |
| ATOM | 1030 | CA | CYS A | 175 | 43.129 | 27.898 | 37.672 | | | 1.00 | 66.01 | | A | C |
| ANISOU | 1030 | CA | CYS A | 175 | 6898 | 8901 | 9283 | −581 | −416 | | | 1270 | A | C |
| ATOM | 1031 | C | CYS A | 175 | 42.662 | 29.242 | 37.120 | | | 1.00 | 57.70 | | A | C |
| ANISOU | 1031 | C | CYS A | 175 | 5628 | 7749 | 8544 | −500 | −432 | | | 1494 | A | C |
| ATOM | 1032 | O | CYS A | 175 | 43.449 | 30.176 | 37.050 | | | 1.00 | 47.99 | | A | O |
| ANISOU | 1032 | O | CYS A | 175 | 4363 | 6347 | 7524 | −442 | −424 | | | 1524 | A | O |
| ATOM | 1033 | CB | CYS A | 175 | 42.466 | 27.637 | 39.013 | | | 1.00 | 63.25 | | A | C |
| ANISOU | 1033 | CB | CYS A | 175 | 6557 | 8539 | 8936 | −547 | −352 | | | 1068 | A | C |
| ATOM | 1034 | SG | CYS A | 175 | 42.297 | 29.145 | 39.959 | | | 1.00 | 61.36 | | A | S |
| ANISOU | 1034 | SG | CYS A | 175 | 6225 | 8034 | 9055 | −391 | −248 | | | 930 | A | S |
| ATOM | 1035 | N | LYS A | 176 | 41.393 | 29.348 | 36.731 | | | 1.00 | 57.88 | | A | N |
| ANISOU | 1035 | N | LYS A | 176 | 5494 | 7882 | 8614 | −505 | −464 | | | 1677 | A | N |
| ATOM | 1036 | CA | LYS A | 176 | 40.874 | 30.605 | 36.183 | | | 1.00 | 55.23 | | A | C |
| ANISOU | 1036 | CA | LYS A | 176 | 4926 | 7472 | 8586 | −432 | −490 | | | 1944 | A | C |
| ATOM | 1037 | C | LYS A | 176 | 39.350 | 30.760 | 36.267 | | | 1.00 | 63.84 | | A | C |
| ANISOU | 1037 | C | LYS A | 176 | 5838 | 8662 | 9757 | −404 | −484 | | | 2068 | A | C |
| ATOM | 1038 | O | LYS A | 176 | 38.629 | 29.791 | 36.085 | | | 1.00 | 73.90 | | A | O |
| ANISOU | 1038 | O | LYS A | 176 | 7174 | 10122 | 10783 | −495 | −505 | | | 2014 | A | O |
| ATOM | 1039 | CB | LYS A | 176 | 41.298 | 30.707 | 34.717 | | | 1.00 | 55.15 | | A | C |
| ANISOU | 1039 | CB | LYS A | 176 | 4886 | 7588 | 8482 | −538 | −601 | | | 2224 | A | C |
| ATOM | 1040 | CG | LYS A | 176 | 41.101 | 32.064 | 34.076 | | | 1.00 | 52.95 | | A | C |
| ANISOU | 1040 | CG | LYS A | 176 | 4368 | 7203 | 8549 | −472 | −643 | | | 2525 | A | C |
| ATOM | 1041 | CD | LYS A | 176 | 41.346 | 31.961 | 32.585 | | | 1.00 | 58.00 | | A | C |
| ANISOU | 1041 | CD | LYS A | 176 | 4995 | 8028 | 9013 | −611 | −755 | | | 2800 | A | C |
| ATOM | 1042 | CE | LYS A | 176 | 41.197 | 33.301 | 31.891 | | | 1.00 | 72.41 | | A | C |
| ANISOU | 1042 | CE | LYS A | 176 | 6594 | 9741 | 11179 | −561 | −809 | | | 3112 | A | C |
| ATOM | 1043 | NZ | LYS A | 176 | 41.327 | 33.159 | 30.416 | | | 1.00 | 71.50 | | A | N1+ |
| ANISOU | 1043 | NZ | LYS A | 176 | 6474 | 9828 | 10863 | −709 | −909 | | | 3382 | A | N1+ |
| ATOM | 1044 | N | CYS A | 177 | 38.862 | 31.969 | 36.552 | | | 1.00 | 66.54 | | A | N |
| ANISOU | 1044 | N | CYS A | 177 | 5945 | 8874 | 10463 | −277 | −458 | | | 2258 | A | N |
| ATOM | 1045 | CA | CYS A | 177 | 37.421 | 32.210 | 36.574 | | | 1.00 | 73.32 | | A | C |
| ANISOU | 1045 | CA | CYS A | 177 | 6575 | 9817 | 11464 | −219 | −437 | | | 2434 | A | C |
| ATOM | 1046 | C | CYS A | 177 | 37.072 | 32.752 | 35.183 | | | 1.00 | 67.92 | | A | C |
| ANISOU | 1046 | C | CYS A | 177 | 5637 | 9201 | 10967 | −243 | −549 | | | 2869 | A | C |
| ATOM | 1047 | O | CYS A | 177 | 37.906 | 33.385 | 34.541 | | | 1.00 | 70.67 | | A | O |
| ANISOU | 1047 | O | CYS A | 177 | 5967 | 9462 | 11421 | −256 | −611 | | | 3008 | A | O |
| ATOM | 1048 | CB | CYS A | 177 | 37.011 | 33.200 | 37.673 | | | 1.00 | 80.74 | | A | C |
| ANISOU | 1048 | CB | CYS A | 177 | 7449 | 10517 | 12711 | 2 | −251 | | | 2235 | A | C |
| ATOM | 1049 | SG | CYS A | 177 | 37.404 | 32.751 | 39.387 | | | 1.00 | 90.22 | | A | S |
| ANISOU | 1049 | SG | CYS A | 177 | 8896 | 11767 | 13615 | −26 | −148 | | | 1808 | A | S |
| ATOM | 1050 | N | PRO A | 178 | 35.840 | 32.512 | 34.711 | | | 1.00 | 63.67 | | A | N |
| ANISOU | 1050 | N | PRO A | 178 | 4885 | 8845 | 10461 | −269 | −592 | | | 3124 | A | N |
| ATOM | 1051 | CA | PRO A | 178 | 35.400 | 32.924 | 33.368 | | | 1.00 | 64.27 | | A | C |
| ANISOU | 1051 | CA | PRO A | 178 | 4729 | 9060 | 10631 | −360 | −746 | | | 3583 | A | C |
| ATOM | 1052 | C | PRO A | 178 | 35.217 | 34.427 | 33.128 | | | 1.00 | 68.37 | | A | C |
| ANISOU | 1052 | C | PRO A | 178 | 5022 | 9316 | 11641 | −172 | −699 | | | 3780 | A | C |
| ATOM | 1053 | O | PRO A | 178 | 35.038 | 35.192 | 34.071 | | | 1.00 | 58.31 | | A | O |
| ANISOU | 1053 | O | PRO A | 178 | 3702 | 7757 | 10695 | 59 | −526 | | | 3603 | A | O |
| ATOM | 1054 | CB | PRO A | 178 | 34.050 | 32.209 | 33.205 | | | 1.00 | 72.69 | | A | C |
| ANISOU | 1054 | CB | PRO A | 178 | 5604 | 10364 | 11652 | −419 | −789 | | | 3793 | A | C |
| ATOM | 1055 | CG | PRO A | 178 | 33.968 | 31.224 | 34.325 | | | 1.00 | 70.63 | | A | C |
| ANISOU | 1055 | CG | PRO A | 178 | 5568 | 10163 | 11105 | −446 | −705 | | | 3418 | A | C |
| ATOM | 1056 | CD | PRO A | 178 | 34.778 | 31.805 | 35.437 | | | 1.00 | 57.85 | | A | C |
| ANISOU | 1056 | CD | PRO A | 178 | 4124 | 8250 | 9606 | −270 | −533 | | | 3030 | A | C |
| ATOM | 1057 | N | SER A | 179 | 35.252 | 34.820 | 31.852 | | | 1.00 | 75.00 | | A | N |
| ANISOU | 1057 | N | SER A | 179 | 5736 | 10246 | 12514 | −286 | −856 | | | 4144 | A | N |
| ATOM | 1058 | CA | SER A | 179 | 35.072 | 36.212 | 31.420 | | | 1.00 | 77.18 | | A | C |
| ANISOU | 1058 | CA | SER A | 179 | 5771 | 10303 | 13251 | −146 | −849 | | | 4429 | A | C |
| ATOM | 1059 | C | SER A | 179 | 33.586 | 36.536 | 31.338 | | | 1.00 | 84.91 | | A | C |
| ANISOU | 1059 | C | SER A | 179 | 6558 | 11293 | 14413 | −77 | −800 | | | 4676 | A | C |
| ATOM | 1060 | O | SER A | 179 | 32.778 | 35.603 | 31.394 | | | 1.00 | 93.34 | | A | O |
| ANISOU | 1060 | O | SER A | 179 | 7611 | 12595 | 15260 | −160 | −821 | | | 4702 | A | O |
| ATOM | 1061 | CB | SER A | 179 | 35.708 | 36.422 | 30.052 | | | 1.00 | 75.06 | | A | C |
| ANISOU | 1061 | CB | SER A | 179 | 5547 | 10127 | 12846 | −332 | −1006 | | | 4701 | A | C |
| ATOM | 1062 | OG | SER A | 179 | 35.151 | 35.532 | 29.103 | | | 1.00 | 72.12 | | A | C |
| ANISOU | 1062 | OG | SER A | 179 | 5163 | 10066 | 12175 | −557 | −1143 | | | 4993 | A | O |
| ATOM | 1063 | N | LYS A | 180 | 33.242 | 37.844 | 31.319 | | | 1.00 | 85.37 | | A | N |
| ANISOU | 1063 | N | LYS A | 180 | 6460 | 11089 | 14888 | 67 | −735 | | | 4876 | A | N |
| ATOM | 1064 | CA | LYS A | 180 | 31.842 | 38.253 | 31.252 | | | 1.00 | 93.10 | | A | C |
| ANISOU | 1064 | CA | LYS A | 180 | 7231 | 12071 | 16073 | 136 | −675 | | | 5147 | A | C |
| ATOM | 1065 | C | LYS A | 180 | 31.115 | 37.485 | 30.153 | | | 1.00 | 80.47 | | A | C |
| ANISOU | 1065 | C | LYS A | 180 | 5617 | 10844 | 14113 | −148 | −863 | | | 5486 | A | C |
| ATOM | 1066 | O | LYS A | 180 | 30.079 | 36.848 | 30.401 | | | 1.00 | 73.21 | | A | O |
| ANISOU | 1066 | O | LYS A | 180 | 4637 | 10107 | 13075 | −178 | −856 | | | 5549 | A | O |
| ATOM | 1067 | CB | LYS A | 180 | 31.741 | 39.761 | 31.025 | | | 1.00 | 105.68 | | A | C |
| ANISOU | 1067 | CB | LYS A | 180 | 8666 | 13335 | 18155 | 284 | −598 | | | 5350 | A | C |
| ATOM | 1068 | CG | LYS A | 180 | 30.329 | 40.304 | 31.192 | | | 1.00 | 111.80 | | A | C |
| ANISOU | 1068 | CG | LYS A | 180 | 9205 | 14049 | 19225 | 418 | −479 | | | 5588 | A | C |
| ATOM | 1069 | CD | LYS A | 180 | 30.255 | 41.820 | 30.983 | | | 1.00 | 115.29 | | A | C |
| ANISOU | 1069 | CD | LYS A | 180 | 9487 | 14143 | 20175 | 560 | −393 | | | 5777 | A | C |
| ATOM | 1070 | CE | LYS A | 180 | 30.854 | 42.596 | 32.153 | | | 1.00 | 114.37 | | A | C |
| ANISOU | 1070 | CE | LYS A | 180 | 9436 | 13608 | 20413 | 852 | −187 | | | 5389 | A | C |
| ATOM | 1071 | NZ | LYS A | 180 | 30.531 | 44.055 | 32.078 | | | 1.00 | 116.90 | | A | N1+ |
| ANISOU | 1071 | NZ | LYS A | 180 | 9590 | 13575 | 21254 | 1011 | −74 | | | 5559 | A | N1+ |
| ATOM | 1072 | N | ASN A | 181 | 31.691 | 37.495 | 28.941 | | | 1.00 | 73.66 | | A | N |
| ANISOU | 1072 | N | ASN A | 181 | 4827 | 10108 | 13052 | −374 | −1033 | | | 5698 | A | N |
| ATOM | 1073 | CA | ASN A | 181 | 31.139 | 36.760 | 27.807 | | | 1.00 | 75.90 | | A | C |
| ANISOU | 1073 | CA | ASN A | 181 | 5137 | 10746 | 12957 | −680 | −1221 | | | 6005 | A | C |
| ATOM | 1074 | C | ASN A | 181 | 30.844 | 35.302 | 28.159 | | | 1.00 | 72.41 | | A | C |
| ANISOU | 1074 | C | ASN A | 181 | 4842 | 10580 | 12092 | −812 | −1280 | | | 5813 | A | C |
| ATOM | 1075 | O | ASN A | 181 | 29.760 | 34.789 | 27.869 | | | 1.00 | 75.78 | | A | O |
| ANISOU | 1075 | O | ASN A | 181 | 5209 | 11215 | 12370 | −950 | −1354 | | | 6032 | A | O |
| ATOM | 1076 | CE | ASN A | 181 | 32.101 | 36.850 | 26.618 | | | 1.00 | 82.18 | | A | C |
| ANISOU | 1076 | CB | ASN A | 181 | 6038 | 11644 | 13541 | −894 | −1360 | | | 6143 | A | C |
| ATOM | 1077 | CG | ASN A | 181 | 32.109 | 38.228 | 25.981 | | | 1.00 | 102.84 | | A | C |
| ANISOU | 1077 | CG | ASN A | 181 | 8473 | 14064 | 16536 | −857 | −1346 | | | 6463 | A | C |
| ATOM | 1078 | OD1 | ASN A | 181 | 31.496 | 39.165 | 26.500 | | | 1.00 | 112.69 | | A | C |
| ANISOU | 1078 | OD1 | ASN A | 181 | 9529 | 15052 | 18234 | −648 | −1221 | | | 6543 | A | O |
| ATOM | 1079 | ND2 | ASN A | 181 | 32.801 | 38.361 | 24.855 | | | 1.00 | 106.55 | | A | N |
| ANISOU | 1079 | ND2 | ASN A | 181 | 8993 | 14661 | 16830 | −1069 | −1461 | | | 6647 | A | N |
| ATOM | 1080 | N | GLN A | 182 | 31.792 | 34.613 | 28.785 | | | 1.00 | 74.11 | | A | N |
| ANISOU | 1080 | N | GLN A | 182 | 5245 | 10797 | 12115 | −789 | −1252 | | | 5418 | A | N |
| ATOM | 1081 | CA | GLN A | 182 | 31.560 | 33.205 | 29.089 | | | 1.00 | 82.34 | | A | C |
| ANISOU | 1081 | CA | GLN A | 182 | 6437 | 12093 | 12756 | −944 | −1314 | | | 5236 | A | C |
| ATOM | 1082 | C | GLN A | 182 | 30.464 | 33.033 | 30.130 | | | 1.00 | 94.59 | | A | C |
| ANISOU | 1082 | C | GLN A | 182 | 7857 | 13622 | 14460 | −798 | −1197 | | | 5178 | A | C |
| ATOM | 1083 | O | GLN A | 182 | 29.760 | 32.017 | 30.125 | | | 1.00 | 108.30 | | A | O |
| ANISOU | 1083 | O | GLN A | 182 | 9647 | 15596 | 15904 | −969 | −1281 | | | 5210 | A | O |
| ATOM | 1084 | CB | GLN A | 182 | 32.848 | 32.543 | 29.568 | | | 1.00 | 76.61 | | A | C |
| ANISOU | 1084 | CB | GLN A | 182 | 5924 | 11360 | 11824 | −954 | −1295 | | | 4838 | A | C |
| ATOM | 1085 | CG | GLN A | 182 | 33.959 | 32.506 | 28.529 | | | 1.00 | 68.61 | | A | C |
| ANISOU | 1085 | CG | GLN A | 182 | 5055 | 10428 | 10587 | −1118 | −1407 | | | 4887 | A | C |
| ATOM | 1086 | CD | GLN A | 182 | 35.307 | 32.146 | 29.143 | | | 1.00 | 65.51 | | A | C |
| ANISOU | 1086 | CD | GLN A | 182 | 4881 | 9921 | 10089 | −1052 | −1318 | | | 4479 | A | C |
| ATOM | 1087 | OE1 | GLN A | 182 | 35.849 | 32.884 | 29.976 | | | 1.00 | 70.21 | | A | O |
| ANISOU | 1087 | OE1 | GLN A | 182 | 5467 | 10218 | 10993 | −821 | −1165 | | | 4278 | A | O |
| ATOM | 1088 | NE2 | GLN A | 182 | 35.856 | 31.005 | 28.740 | | | 1.00 | 56.66 | | A | N |
| ANISOU | 1088 | NE2 | GLN A | 182 | 4065 | 8976 | 8487 | −1252 | −1373 | | | 4307 | A | N |
| ATOM | 1089 | N | LEU A | 183 | 30.293 | 34.002 | 31.032 | | | 1.00 | 91.16 | | A | N |
| ANISOU | 1089 | N | LEU A | 183 | 7257 | 12906 | 14474 | −491 | −996 | | | 5093 | A | N |
| ATOM | 1090 | CA | LEU A | 183 | 29.198 | 33.892 | 31.994 | | | 1.00 | 85.89 | | A | C |
| ANISOU | 1090 | CA | LEU A | 183 | 6443 | 12236 | 13954 | −338 | −857 | | | 5062 | A | C |
| ATOM | 1091 | C | LEU A | 183 | 27.841 | 34.054 | 31.297 | | | 1.00 | 85.84 | | A | C |
| ANISOU | 1091 | C | LEU A | 183 | 6258 | 12368 | 13990 | −429 | −932 | | | 5515 | A | C |
| ATOM | 1092 | O | LEU A | 183 | 26.934 | 33.240 | 31.499 | | | 1.00 | 86.51 | | A | O |
| ANISOU | 1092 | O | LEU A | 183 | 6319 | 12666 | 13886 | −529 | −971 | | | 5588 | A | O |
| ATOM | 1093 | CE | LEU A | 183 | 29.388 | 34.901 | 33.136 | | | 1.00 | 83.75 | | A | C |
| ANISOU | 1093 | CB | LEU A | 183 | 6061 | 11619 | 14140 | 18 | −597 | | | 4832 | A | C |
| ATOM | 1094 | CG | LEU A | 183 | 30.509 | 34.593 | 34.150 | | | 1.00 | 69.40 | | A | C |
| ANISOU | 1094 | CG | LEU A | 183 | 4399 | 9685 | 12284 | 115 | −491 | | | 4363 | A | C |
| ATOM | 1095 | CD1 | LEU A | 183 | 30.735 | 35.717 | 35.176 | | | 1.00 | 69.50 | | A | C |
| ANISOU | 1095 | CD1 | LEU A | 183 | 4329 | 9316 | 12761 | 456 | −238 | | | 4147 | A | C |
| ATOM | 1096 | CD2 | LEU A | 183 | 30.211 | 33.299 | 34.877 | | | 1.00 | 62.96 | | A | C |
| ANISOU | 1096 | CD2 | LEU A | 183 | 3661 | 9112 | 11148 | 17 | −482 | | | 4158 | A | C |
| ATOM | 1097 | N | ASN A | 184 | 27.707 | 35.056 | 30.410 | | | 1.00 | 78.48 | | A | N |
| ANISOU | 1097 | N | ASN A | 184 | 5202 | 11331 | 13286 | −432 | −971 | | | 5851 | A | N |
| ATOM | 1098 | CA | ASN A | 184 | 26.456 | 35.222 | 29.662 | | | 1.00 | 77.04 | | A | C |
| ANISOU | 1098 | CA | ASN A | 184 | 4838 | 11289 | 13144 | −552 | −1055 | | | 6327 | A | C |
| ATOM | 1099 | C | ASN A | 184 | 26.118 | 33.978 | 28.838 | | | 1.00 | 76.55 | | A | C |
| ANISOU | 1099 | C | ASN A | 184 | 4917 | 11598 | 12571 | −924 | −1292 | | | 6487 | A | C |
| ATOM | 1100 | O | ASN A | 184 | 24.959 | 33.562 | 28.797 | | | 1.00 | 80.77 | | A | O |
| ANISOU | 1100 | O | ASN A | 184 | 5347 | 12304 | 13039 | −1018 | −1338 | | | 6730 | A | O |
| ATOM | 1101 | CB | ASN A | 184 | 26.516 | 36.466 | 28.766 | | | 1.00 | 87.69 | | A | C |
| ANISOU | 1101 | CB | ASN A | 184 | 6044 | 12475 | 14798 | −541 | −1071 | | | 6665 | A | C |
| ATOM | 1102 | CG | ASN A | 184 | 25.140 | 36.836 | 28.159 | | | 1.00 | 107.46 | | A | C |
| ANISOU | 1102 | CG | ASN A | 184 | 8300 | 15076 | 17454 | −619 | −1111 | | | 7183 | A | C |
| ATOM | 1103 | OD1 | ASN A | 184 | 24.950 | 36.785 | 26.943 | | | 1.00 | 116.57 | | A | O |
| ANISOU | 1103 | OD1 | ASN A | 184 | 9442 | 16411 | 18438 | −899 | −1294 | | | 7548 | A | O |
| ATOM | 1104 | ND2 | ASN A | 184 | 24.189 | 37.229 | 29.013 | | | 1.00 | 107.94 | | A | N |
| ANISOU | 1104 | ND2 | ASN A | 184 | 8154 | 15019 | 17839 | −373 | −926 | | | 7221 | A | N |
| ATOM | 1105 | N | LYS A | 185 | 27.111 | 33.364 | 28.182 | | | 1.00 | 79.07 | | A | N |
| ANISOU | 1105 | N | LYS A | 185 | 5479 | 12041 | 12524 | −1147 | −1438 | | | 6356 | A | N |
| ATOM | 1106 | CA | LYS A | 185 | 26.912 | 32.079 | 27.494 | | | 1.00 | 80.45 | | A | C |
| ANISOU | 1106 | CA | LYS A | 185 | 5842 | 12545 | 12182 | −1512 | −1641 | | | 6417 | A | C |
| ATOM | 1107 | C | LYS A | 185 | 26.541 | 30.944 | 28.440 | | | 1.00 | 71.90 | | A | C |
| ANISOU | 1107 | C | LYS A | 185 | 4855 | 11574 | 10890 | −1525 | −1624 | | | 6158 | A | C |
| ATOM | 1108 | O | LYS A | 185 | 26.224 | 29.843 | 27.979 | | | 1.00 | 71.82 | | A | O |
| ANISOU | 1108 | O | LYS A | 185 | 5002 | 11810 | 10476 | −1834 | −1782 | | | 6207 | A | O |
| ATOM | 1109 | CB | LYS A | 185 | 28.174 | 31.616 | 26.742 | | | 1.00 | 85.85 | | A | C |
| ANISOU | 1109 | CB | LYS A | 185 | 6783 | 13317 | 12517 | −1719 | −1752 | | | 6249 | A | C |
| ATOM | 1110 | CG | LYS A | 185 | 28.488 | 32.285 | 25.405 | | | 1.00 | 92.80 | | A | C |
| ANISOU | 1110 | CG | LYS A | 185 | 7626 | 14242 | 13391 | −1890 | −1843 | | | 6563 | A | C |
| ATOM | 1111 | CD | LYS A | 185 | 29.791 | 31.714 | 24.839 | | | 1.00 | 77.13 | | A | C |
| ANISOU | 1111 | CD | LYS A | 185 | 5896 | 12362 | 11048 | −2070 | −1910 | | | 6327 | A | C |
| ATOM | 1112 | CE | LYS A | 185 | 30.209 | 32.415 | 23.566 | | | 1.00 | 72.78 | | A | C |
| ANISOU | 1112 | CE | LYS A | 185 | 5289 | 11863 | 10500 | −2206 | −1978 | | | 6537 | A | C |
| ATOM | 1113 | NZ | LYS A | 185 | 31.489 | 31.872 | 23.067 | | | 1.00 | 79.48 | | A | N1+ |
| ANISOU | 1113 | NZ | LYS A | 185 | 6357 | 12821 | 11019 | −2346 | −2018 | | | 6265 | A | N1+ |
| ATOM | 1114 | N | GLY A | 186 | 26.624 | 31.154 | 29.742 | | | 1.00 | 70.63 | | A | N |
| ANISOU | 1114 | N | GLY A | 186 | 4621 | 11238 | 10979 | −1227 | −1429 | | | 5869 | A | N |
| ATOM | 1115 | CA | GLY A | 186 | 26.231 | 30.118 | 30.660 | | | 1.00 | 70.28 | | A | C |
| ANISOU | 1115 | CA | GLY A | 186 | 4643 | 11311 | 10750 | −1249 | −1403 | | | 5651 | A | C |
| ATOM | 1116 | C | GLY A | 186 | 27.341 | 29.201 | 31.096 | | | 1.00 | 65.52 | | A | C |
| ANISOU | 1116 | C | GLY A | 186 | 4307 | 10733 | 9854 | −1325 | −1418 | | | 5219 | A | C |
| ATOM | 1117 | O | GLY A | 186 | 27.051 | 28.125 | 31.640 | | | 1.00 | 64.43 | | A | O |
| ANISOU | 1117 | O | GLY A | 186 | 4278 | 10735 | 9469 | −1439 | −1450 | | | 5067 | A | O |
| ATOM | 1118 | N | ILE A | 187 | 28.597 | 29.588 | 30.867 | | | 1.00 | 65.47 | | A | N |
| ANISOU | 1118 | N | ILE A | 187 | 4408 | 10594 | 9872 | −1276 | −1400 | | | 5037 | A | N |
| ATOM | 1119 | CA | ILE A | 187 | 29.761 | 28.855 | 31.363 | | | 1.00 | 73.01 | | A | C |
| ANISOU | 1119 | CA | ILE A | 187 | 5591 | 11539 | 10611 | −1312 | −1384 | | | 4629 | A | C |
| ATOM | 1120 | C | ILE A | 187 | 29.946 | 29.228 | 32.831 | | | 1.00 | 77.96 | | A | C |
| ANISOU | 1120 | C | ILE A | 187 | 6111 | 11970 | 11540 | −1014 | −1150 | | | 4333 | A | C |
| ATOM | 1121 | O | ILE A | 187 | 30.180 | 30.397 | 33.160 | | | 1.00 | 84.30 | | A | O |
| ANISOU | 1121 | O | ILE A | 187 | 6762 | 12531 | 12738 | −754 | −999 | | | 4322 | A | O |
| ATOM | 1122 | CB | ILE A | 187 | 31.027 | 29.189 | 30.545 | | | 1.00 | 69.03 | | A | C |
| ANISOU | 1122 | CB | ILE A | 187 | 5215 | 10976 | 10036 | −1367 | −1443 | | | 4589 | A | C |
| ATOM | 1123 | CG1 | ILE A | 187 | 30.790 | 29.057 | 29.028 | | | 1.00 | 61.27 | | A | C |
| ANISOU | 1123 | CG1 | ILE A | 187 | 4300 | 10185 | 8794 | −1648 | −1641 | | | 4923 | A | C |
| ATOM | 1124 | CG2 | ILE A | 187 | 32.223 | 28.355 | 30.985 | | | 1.00 | 58.51 | | A | C |
| ANISOU | 1124 | CG2 | ILE A | 187 | 4185 | 9612 | 8433 | −1412 | −1408 | | | 4167 | A | C |
| ATOM | 1125 | CD1 | ILE A | 187 | 30.728 | 27.630 | 28.502 | | | 1.00 | 60.46 | | A | C |
| ANISOU | 1125 | CD1 | ILE A | 187 | 4467 | 10338 | 8170 | −1995 | −1807 | | | 4861 | A | C |
| ATOM | 1126 | N | LYS A | 188 | 29.814 | 28.248 | 33.722 | | | 1.00 | 72.39 | | A | N |
| ANISOU | 1126 | N | LYS A | 188 | 5492 | 11363 | 10651 | −1064 | −1117 | | | 4097 | A | N |
| ATOM | 1127 | CA | LYS A | 188 | 30.026 | 28.530 | 35.133 | | | 1.00 | 72.37 | | A | C |
| ANISOU | 1127 | CA | LYS A | 188 | 5504 | 11153 | 10839 | −797 | −864 | | | 3756 | A | C |
| ATOM | 1128 | C | LYS A | 188 | 31.358 | 28.002 | 35.655 | | | 1.00 | 62.02 | | A | C |
| ANISOU | 1128 | C | LYS A | 188 | 4584 | 9693 | 9289 | −802 | −799 | | | 3292 | A | C |
| ATOM | 1129 | O | LYS A | 188 | 31.783 | 28.403 | 36.744 | | | 1.00 | 54.58 | | A | O |
| ANISOU | 1129 | O | LYS A | 188 | 3698 | 8541 | 8500 | −592 | −599 | | | 2991 | A | O |
| ATOM | 1130 | CB | LYS A | 188 | 28.860 | 27.974 | 35.962 | | | 1.00 | 77.56 | | A | C |
| ANISOU | 1130 | CB | LYS A | 188 | 6003 | 11977 | 11489 | −789 | −809 | | | 3804 | A | C |
| ATOM | 1131 | CG | LYS A | 188 | 27.510 | 28.520 | 35.480 | | | 1.00 | 89.47 | | A | C |
| ANISOU | 1131 | CG | LYS A | 188 | 7242 | 13559 | 13195 | −747 | −835 | | | 4227 | A | C |
| ATOM | 1132 | CD | LYS A | 188 | 26.347 | 28.191 | 36.396 | | | 1.00 | 92.88 | | A | C |
| ANISOU | 1132 | CD | LYS A | 188 | 7521 | 14101 | 13669 | −669 | −721 | | | 4267 | A | C |
| ATOM | 1133 | CE | LYS A | 188 | 26.032 | 26.702 | 36.372 | | | 1.00 | 94.49 | | A | C |
| ANISOU | 1133 | CE | LYS A | 188 | 7902 | 14562 | 13436 | −975 | −902 | | | 4250 | A | C |
| ATOM | 1134 | NZ | LYS A | 188 | 25.328 | 26.308 | 35.117 | | | 1.00 | 99.84 | | A | N1+ |
| ANISOU | 1134 | NZ | LYS A | 188 | 8624 | 15407 | 13905 | −1245 | −1152 | | | 4624 | A | N1+ |
| ATOM | 1135 | N | TYR A | 189 | 32.053 | 27.161 | 34.893 | | | 1.00 | 60.77 | | A | N |
| ANISOU | 1135 | N | TYR A | 189 | 4695 | 9628 | 8768 | −1034 | −954 | | | 3235 | A | N |
| ATOM | 1136 | CA | TYR A | 189 | 33.314 | 26.634 | 35.384 | | | 1.00 | 54.85 | | A | C |
| ANISOU | 1136 | CA | TYR A | 189 | 4283 | 8740 | 7817 | −1025 | −884 | | | 2833 | A | C |
| ATOM | 1137 | C | TYR A | 189 | 34.344 | 26.543 | 34.269 | | | 1.00 | 53.00 | | A | C |
| ANISOU | 1137 | C | TYR A | 189 | 4243 | 8499 | 7396 | −1147 | −986 | | | 2851 | A | C |
| ATOM | 1138 | O | TYR A | 189 | 34.051 | 26.018 | 33.195 | | | 1.00 | 51.29 | | A | C |
| ANISOU | 1138 | O | TYR A | 189 | 4068 | 8480 | 6940 | −1372 | −1158 | | | 3061 | A | C |
| ATOM | 1139 | CB | TYR A | 189 | 33.092 | 25.260 | 36.015 | | | 1.00 | 47.86 | | A | C |
| ANISOU | 1139 | CB | TYR A | 189 | 3588 | 7981 | 6615 | −1168 | −915 | | | 2642 | A | C |
| ATOM | 1140 | CG | TYR A | 189 | 32.078 | 25.277 | 37.146 | | | 1.00 | 48.77 | | A | C |
| ANISOU | 1140 | CG | TYR A | 189 | 3510 | 8142 | 6879 | −1058 | −804 | | | 2631 | A | C |
| ATOM | 1141 | CD1 | TYR A | 189 | 32.314 | 25.999 | 38.307 | | | 1.00 | 52.31 | | A | C |
| ANISOU | 1141 | CD1 | TYR A | 189 | 3908 | 8396 | 7572 | −805 | −583 | | | 2402 | A | C |
| ATOM | 1142 | CD2 | TYR A | 189 | 30.903 | 24.558 | 37.057 | | | 1.00 | 52.35 | | A | C |
| ANISOU | 1142 | CD2 | TYR A | 189 | 3841 | 8840 | 7210 | −1223 | −919 | | | 2849 | A | C |
| ATOM | 1143 | CE1 | TYR A | 189 | 31.398 | 26.015 | 39.331 | | | 1.00 | 64.50 | | A | C |
| ANISOU | 1143 | CE1 | TYR A | 189 | 5284 | 9997 | 9227 | −697 | −453 | | | 2383 | A | C |
| ATOM | 1144 | CE2 | TYR A | 189 | 29.969 | 24.567 | 38.083 | | | 1.00 | 63.04 | | A | C |
| ANISOU | 1144 | CE2 | TYR A | 189 | 4995 | 10263 | 8695 | −1117 | −801 | | | 2864 | A | C |
| ATOM | 1145 | CZ | TYR A | 189 | 30.218 | 25.298 | 39.220 | | | 1.00 | 65.24 | | A | C |
| ANISOU | 1145 | CZ | TYR A | 189 | 5226 | 10354 | 9208 | −843 | −554 | | | 2624 | A | C |
| ATOM | 1146 | OH | TYR A | 189 | 29.297 | 25.297 | 40.246 | | | 1.00 | 62.96 | | A | O |
| ANISOU | 1146 | OH | TYR A | 189 | 4748 | 10150 | 9024 | −732 | −409 | | | 2627 | A | O |
| ATOM | 1147 | N | LEU A | 190 | 35.577 | 26.917 | 34.598 | | | 1.00 | 52.03 | | A | N |
| ANISOU | 1147 | N | LEU A | 190 | 4237 | 8159 | 7371 | −1009 | −878 | | | 2639 | A | N |
| ATOM | 1148 | CA | LEU A | 190 | 36.736 | 26.753 | 33.730 | | | 1.00 | 49.76 | | A | C |
| ANISOU | 1148 | CA | LEU A | 190 | 4184 | 7867 | 6857 | −1107 | −927 | | | 2573 | A | C |
| ATOM | 1149 | C | LEU A | 190 | 37.484 | 25.706 | 34.553 | | | 1.00 | 50.35 | | A | C |
| ANISOU | 1149 | C | LEU A | 190 | 4550 | 7880 | 6699 | −1118 | −856 | | | 2206 | A | C |
| ATOM | 1150 | O | LEU A | 190 | 38.005 | 26.013 | 35.619 | | | 1.00 | 59.49 | | A | O |
| ANISOU | 1150 | O | LEU A | 190 | 5744 | 8851 | 8010 | −962 | −727 | | | 1973 | A | O |
| ATOM | 1151 | CB | LEU A | 190 | 37.537 | 28.040 | 33.617 | | | 1.00 | 49.86 | | A | C |
| ANISOU | 1151 | CB | LEU A | 190 | 4107 | 7697 | 7141 | −966 | −872 | | | 2636 | A | C |
| ATOM | 1152 | CG | LEU A | 190 | 36.985 | 29.101 | 32.669 | | | 1.00 | 48.62 | | A | C |
| ANISOU | 1152 | CG | LEU A | 190 | 3647 | 7542 | 7283 | −922 | −931 | | | 3007 | A | C |
| ATOM | 1153 | CD1 | LEU A | 190 | 37.837 | 30.357 | 32.717 | | | 1.00 | 48.76 | | A | C |
| ANISOU | 1153 | CD1 | LEU A | 190 | 3632 | 7368 | 7526 | −813 | −891 | | | 3042 | A | C |
| ATOM | 1154 | CD2 | LEU A | 190 | 36.923 | 28.549 | 31.259 | | | 1.00 | 59.49 | | A | C |
| ANISOU | 1154 | CD2 | LEU A | 190 | 5001 | 9200 | 8405 | −1161 | −1112 | | | 3307 | A | C |
| ATOM | 1155 | N | ILE A | 191 | 37.534 | 24.474 | 34.059 | | | 1.00 | 43.06 | | A | N |
| ANISOU | 1155 | N | ILE A | 191 | 3843 | 7106 | 5412 | −1313 | −947 | | | 2164 | A | N |
| ATOM | 1156 | CA | ILE A | 191 | 38.080 | 23.350 | 34.816 | | | 1.00 | 41.24 | | A | C |
| ANISOU | 1156 | CA | ILE A | 191 | 3870 | 6834 | 4967 | −1341 | −898 | | | 1860 | A | C |
| ATOM | 1157 | C | ILE A | 191 | 39.513 | 23.103 | 34.358 | | | 1.00 | 44.18 | | A | C |
| ANISOU | 1157 | C | ILE A | 191 | 4467 | 7119 | 5201 | −1328 | −849 | | | 1724 | A | C |
| ATOM | 1158 | O | ILE A | 191 | 39.759 | 22.857 | 33.178 | | | 1.00 | 63.43 | | A | O |
| ANISOU | 1158 | O | ILE A | 191 | 7008 | 9653 | 7441 | −1445 | −913 | | | 1839 | A | O |
| ATOM | 1159 | CB | ILE A | 191 | 37.213 | 22.092 | 34.634 | | | 1.00 | 41.71 | | A | C |
| ANISOU | 1159 | CB | ILE A | 191 | 4045 | 7073 | 4731 | −1560 | −1024 | | | 1893 | A | C |
| ATOM | 1160 | CG1 | ILE A | 191 | 35.798 | 22.304 | 35.201 | | | 1.00 | 48.59 | | A | C |
| ANISOU | 1160 | CG1 | ILE A | 191 | 4660 | 8050 | 5753 | −1564 | −1060 | | | 2049 | A | C |
| ATOM | 1161 | CG2 | ILE A | 191 | 37.880 | 20.886 | 35.240 | | | 1.00 | 40.88 | | A | C |
| ANISOU | 1161 | CG2 | ILE A | 191 | 4225 | 6905 | 4404 | −1600 | −987 | | | 1606 | A | C |
| ATOM | 1162 | CD1 | ILE A | 191 | 35.040 | 21.017 | 35.506 | | | 1.00 | 43.05 | | A | C |
| ANISOU | 1162 | CD1 | ILE A | 191 | 4067 | 7487 | 4801 | −1756 | −1162 | | | 2022 | A | C |
| ATOM | 1163 | N | THR A | 192 | 40.453 | 23.138 | 35.279 | | | 1.00 | 54.21 | | A | N |
| ANISOU | 1163 | N | THR A | 192 | 5810 | 8222 | 6564 | −1194 | −732 | | | 1493 | A | N |
| ATOM | 1164 | CA | THR A | 192 | 41.850 | 22.964 | 34.928 | | | 1.00 | 37.84 | | A | C |
| ANISOU | 1164 | CA | THR A | 192 | 3898 | 6069 | 4409 | −1158 | −671 | | | 1406 | A | C |
| ATOM | 1165 | C | THR A | 192 | 42.112 | 21.488 | 34.716 | | | 1.00 | 37.19 | | A | C |
| ANISOU | 1165 | C | THR A | 192 | 4098 | 6030 | 4001 | −1265 | −675 | | | 1251 | A | C |
| ATOM | 1166 | O | THR A | 192 | 41.721 | 20.664 | 35.517 | | | 1.00 | 36.39 | | A | O |
| ANISOU | 1166 | O | THR A | 192 | 4074 | 5913 | 3841 | −1289 | −675 | | | 1098 | A | O |
| ATOM | 1167 | CB | THR A | 192 | 42.770 | 23.641 | 35.946 | | | 1.00 | 36.84 | | A | C |
| ANISOU | 1167 | CB | THR A | 192 | 3714 | 5750 | 4534 | −991 | −567 | | | 1267 | A | C |
| ATOM | 1168 | OG1 | THR A | 192 | 42.544 | 23.095 | 37.239 | | | 1.00 | 37.49 | | A | O |
| ANISOU | 1168 | OG1 | THR A | 192 | 3994 | 5771 | 4480 | −984 | −508 | | | 1056 | A | O |
| ATOM | 1169 | CG2 | THR A | 192 | 42.518 | 25.147 | 35.952 | | | 1.00 | 37.08 | | A | C |
| ANISOU | 1169 | CG2 | THR A | 192 | 3572 | 5717 | 4801 | −911 | −543 | | | 1227 | A | C |
| ATOM | 1170 | N | TYR A | 193 | 42.805 | 21.173 | 33.631 | | | 1.00 | 37.82 | | A | N |
| ANISOU | 1170 | N | TYR A | 193 | 4334 | 6167 | 3870 | −1329 | −673 | | | 1301 | A | N |
| ATOM | 1171 | CA | TYR A | 193 | 42.999 | 19.788 | 33.209 | | | 1.00 | 38.00 | | A | C |
| ANISOU | 1171 | CA | TYR A | 193 | 4651 | 6229 | 3560 | −1446 | −677 | | | 1182 | A | C |
| ATOM | 1172 | C | TYR A | 193 | 44.421 | 19.603 | 32.695 | | | 1.00 | 46.25 | | A | C |
| ANISOU | 1172 | C | TYR A | 193 | 5845 | 7206 | 4524 | −1357 | −546 | | | 1113 | A | C |
| ATOM | 1173 | O | TYR A | 193 | 44.898 | 20.407 | 31.893 | | | 1.00 | 49.56 | | A | O |
| ANISOU | 1173 | O | TYR A | 193 | 6175 | 7664 | 4992 | −1315 | −515 | | | 1259 | A | O |
| ATOM | 1174 | CB | TYR A | 193 | 42.000 | 19.422 | 32.106 | | | 1.00 | 39.82 | | A | C |
| ANISOU | 1174 | CB | TYR A | 193 | 4949 | 6637 | 3544 | −1660 | −814 | | | 1338 | A | C |
| ATOM | 1175 | CG | TYR A | 193 | 42.226 | 18.071 | 31.470 | | | 1.00 | 40.61 | | A | C |
| ANISOU | 1175 | CG | TYR A | 193 | 5397 | 6757 | 3277 | −1799 | −817 | | | 1210 | A | C |
| ATOM | 1176 | CD1 | TYR A | 193 | 41.845 | 16.911 | 32.123 | | | 1.00 | 40.22 | | A | C |
| ANISOU | 1176 | CD1 | TYR A | 193 | 5519 | 6659 | 3105 | −1883 | −859 | | | 1051 | A | C |
| ATOM | 1177 | CD2 | TYR A | 193 | 42.822 | 17.948 | 30.219 | | | 1.00 | 42.03 | | A | C |
| ANISOU | 1177 | CD2 | TYR A | 193 | 5745 | 6996 | 3228 | −1847 | −771 | | | 1246 | A | C |
| ATOM | 1178 | CE1 | TYR A | 193 | 42.055 | 15.664 | 31.546 | | | 1.00 | 42.08 | | A | C |
| ANISOU | 1178 | CE1 | TYR A | 193 | 6101 | 6868 | 3021 | −2008 | −858 | | | 919 | A | C |
| ATOM | 1179 | CE2 | TYR A | 193 | 43.054 | 16.698 | 29.650 | | | 1.00 | 43.13 | | A | C |
| ANISOU | 1179 | CE2 | TYR A | 193 | 6241 | 7122 | 3026 | −1961 | −745 | | | 1093 | A | C |
| ATOM | 1180 | CZ | TYR A | 193 | 42.658 | 15.563 | 30.319 | | | 1.00 | 42.78 | | A | C |
| ANISOU | 1180 | CZ | TYR A | 193 | 6375 | 6993 | 2886 | −2039 | −793 | | | 925 | A | C |
| ATOM | 1181 | OH | TYR A | 193 | 42.873 | 14.306 | 29.797 | | | 1.00 | 44.14 | | A | O |
| ANISOU | 1181 | OH | TYR A | 193 | 6923 | 7106 | 2743 | −2149 | −766 | | | 758 | A | O |
| ATOM | 1182 | N | VAL A | 194 | 45.115 | 18.559 | 33.141 | | | 1.00 | 51.86 | | A | N |
| ANISOU | 1182 | N | VAL A | 194 | 6761 | 7819 | 5123 | −1321 | −464 | | | 917 | A | N |
| ATOM | 1183 | CA | VAL A | 194 | 46.489 | 18.347 | 32.692 | | | 1.00 | 45.92 | | A | C |
| ANISOU | 1183 | CA | VAL A | 194 | 6127 | 7004 | 4317 | −1210 | −313 | | | 870 | A | C |
| ATOM | 1184 | C | VAL A | 194 | 46.466 | 17.418 | 31.483 | | | 1.00 | 39.33 | | A | C |
| ANISOU | 1184 | C | VAL A | 194 | 5572 | 6244 | 3128 | −1322 | −288 | | | 843 | A | C |
| ATOM | 1185 | O | VAL A | 194 | 46.066 | 16.253 | 31.582 | | | 1.00 | 39.54 | | A | O |
| ANISOU | 1185 | O | VAL A | 194 | 5828 | 6237 | 2958 | −1422 | −319 | | | 706 | A | O |
| ATOM | 1186 | CB | VAL A | 194 | 47.379 | 17.803 | 33.817 | | | 1.00 | 44.75 | | A | C |
| ANISOU | 1186 | CE | VAL A | 194 | 6023 | 6701 | 4281 | −1086 | −221 | | | 706 | A | C |
| ATOM | 1187 | CG1 | VAL A | 194 | 48.585 | 17.068 | 33.240 | | | 1.00 | 40.42 | | A | C |
| ANISOU | 1187 | CG1 | VAL A | 194 | 5664 | 6097 | 3596 | −997 | −59 | | | 647 | A | C |
| ATOM | 1188 | CG2 | VAL A | 194 | 47.871 | 18.956 | 34.684 | | | 1.00 | 44.46 | | A | C |
| ANISOU | 1188 | CG2 | VAL A | 194 | 5732 | 6595 | 4567 | −966 | −209 | | | 755 | A | C |
| ATOM | 1189 | N | TRP A | 195 | 46.906 | 17.945 | 30.344 | | | 1.00 | 45.74 | | A | N |
| ANISOU | 1189 | N | TRP A | 195 | 6375 | 7153 | 3853 | −1315 | −232 | | | 974 | A | N |
| ATOM | 1190 | CA | TRP A | 195 | 46.798 | 17.271 | 29.062 | | | 1.00 | 42.69 | | A | C |
| ANISOU | 1190 | CA | TRP A | 195 | 6250 | 6871 | 3098 | −1446 | −211 | | | 966 | A | C |
| ATOM | 1191 | C | TRP A | 195 | 47.727 | 16.072 | 28.989 | | | 1.00 | 52.83 | | A | C |
| ANISOU | 1191 | C | TRP A | 195 | 7833 | 8033 | 4207 | −1360 | −30 | | | 753 | A | C |
| ATOM | 1192 | O | TRP A | 195 | 48.924 | 16.181 | 29.264 | | | 1.00 | 61.98 | | A | O |
| ANISOU | 1192 | O | TRP A | 195 | 8938 | 9103 | 5510 | −1159 | 141 | | | 728 | A | O |
| ATOM | 1193 | CB | TRP A | 195 | 47.116 | 18.270 | 27.959 | | | 1.00 | 44.02 | | A | C |
| ANISOU | 1193 | CB | TRP A | 195 | 6292 | 7191 | 3242 | −1447 | −185 | | | 1183 | A | C |
| ATOM | 1194 | CG | TRP A | 195 | 46.815 | 17.824 | 26.545 | | | 1.00 | 55.90 | | A | C |
| ANISOU | 1194 | CG | TRP A | 195 | 8034 | 8862 | 4344 | −1631 | −198 | | | 1222 | A | C |
| ATOM | 1195 | CD1 | TRP A | 195 | 47.702 | 17.288 | 25.662 | | | 1.00 | 59.43 | | A | C |
| ANISOU | 1195 | CD1 | TRP A | 195 | 8721 | 9336 | 4523 | −1591 | −11 | | | 1141 | A | C |
| ATOM | 1196 | CD2 | TRP A | 195 | 45.561 | 17.929 | 25.838 | | | 1.00 | 59.45 | | A | C |
| ANISOU | 1196 | CD2 | TRP A | 195 | 8496 | 9488 | 4605 | −1891 | −404 | | | 1368 | A | C |
| ATOM | 1197 | NE1 | TRP A | 195 | 47.086 | 17.033 | 24.463 | | | 1.00 | 68.01 | | A | N |
| ANISOU | 1197 | NE1 | TRP A | 195 | 10003 | 10601 | 5238 | −1822 | −87 | | | 1196 | A | N |
| ATOM | 1198 | CE2 | TRP A | 195 | 45.773 | 17.418 | 24.537 | | | 1.00 | 64.48 | | A | C |
| ANISOU | 1198 | CE2 | TRP A | 195 | 9415 | 10254 | 4832 | −2023 | −344 | | | 1350 | A | C |
| ATOM | 1199 | CE3 | TRP A | 195 | 44.284 | 18.389 | 26.180 | | | 1.00 | 65.27 | | A | C |
| ANISOU | 1199 | CE3 | TRP A | 195 | 9029 | 10294 | 5477 | −2025 | −626 | | | 1523 | A | C |
| ATOM | 1200 | CZ2 | TRP A | 195 | 44.757 | 17.358 | 23.571 | | | 1.00 | 53.06 | | A | C |
| ANISOU | 1200 | CZ2 | TRP A | 195 | 8043 | 8985 | 3132 | −2296 | −524 | | | 1471 | A | C |
| ATOM | 1201 | CZ3 | TRP A | 195 | 43.262 | 18.325 | 25.208 | | | 1.00 | 66.43 | | A | C |
| ANISOU | 1201 | CZ3 | TRP A | 195 | 9235 | 10644 | 5360 | −2303 | −807 | | | 1690 | A | C |
| ATOM | 1202 | CH2 | TRP A | 195 | 43.516 | 17.817 | 23.923 | | | 1.00 | 60.06 | | A | C |
| ANISOU | 1202 | CH2 | TRP A | 195 | 8692 | 9925 | 4202 | −2424 | −759 | | | 1641 | A | C |
| ATOM | 1203 | N | GLN A | 196 | 47.170 | 14.928 | 28.614 | | | 1.00 | 47.18 | | A | N |
| ANISOU | 1203 | N | GLN A | 196 | 7429 | 7303 | 3194 | −1516 | −70 | | | 614 | A | N |
| ATOM | 1204 | CA | GLN A | 196 | 47.888 | 13.665 | 28.606 | | | 1.00 | 56.27 | | A | C |
| ANISOU | 1204 | CA | GLN A | 196 | 8896 | 8294 | 4192 | −1439 | 95 | | | 392 | A | C |
| ATOM | 1205 | C | GLN A | 196 | 48.454 | 13.347 | 27.225 | | | 1.00 | 74.85 | | A | C |
| ANISOU | 1205 | C | GLN A | 196 | 11508 | 10711 | 6221 | −1446 | 261 | | | 354 | A | C |
| ATOM | 1206 | O | GLN A | 196 | 48.047 | 13.913 | 26.202 | | | 1.00 | 69.83 | | A | O |
| ANISOU | 1206 | O | GLN A | 196 | 10863 | 10271 | 5399 | −1588 | 197 | | | 491 | A | O |
| ATOM | 1207 | CB | GLN A | 196 | 46.976 | 12.513 | 29.044 | | | 1.00 | 55.67 | | A | C |
| ANISOU | 1207 | CB | GLN A | 196 | 9048 | 8122 | 3980 | −1612 | −41 | | | 240 | A | C |
| ATOM | 1208 | CG | GLN A | 196 | 46.531 | 12.514 | 30.503 | | | 1.00 | 43.32 | | A | C |
| ANISOU | 1208 | CG | GLN A | 196 | 7288 | 6479 | 2694 | −1591 | −160 | | | 233 | A | C |
| ATOM | 1209 | CD | GLN A | 196 | 47.678 | 12.692 | 31.466 | | | 1.00 | 49.26 | | A | C |
| ANISOU | 1209 | CD | GLN A | 196 | 7883 | 7097 | 3738 | −1337 | −10 | | | 199 | A | C |
| ATOM | 1210 | OE1 | GLN A | 196 | 48.449 | 11.766 | 31.713 | | | 1.00 | 57.39 | | A | O |
| ANISOU | 1210 | OE1 | GLN A | 196 | 9089 | 7961 | 4757 | −1228 | 126 | | | 63 | A | O |
| ATOM | 1211 | NE2 | GLN A | 196 | 47.796 | 13.890 | 32.025 | | | 1.00 | 53.64 | | A | N |
| ANISOU | 1211 | NE2 | GLN A | 196 | 8104 | 7713 | 4564 | −1251 | −41 | | | 335 | A | N |
| ATOM | 1212 | N | ASP A | 197 | 49.405 | 12.409 | 27.220 | | | 1.00 | 91.15 | | A | N |
| ANISOU | 1212 | N | ASP A | 197 | 13805 | 12607 | 8220 | −1288 | 486 | | | 172 | A | N |
| ATOM | 1213 | CA | ASP A | 197 | 49.941 | 11.846 | 25.988 | | | 1.00 | 98.99 | | A | C |
| ANISOU | 1213 | CA | ASP A | 197 | 15119 | 13621 | 8871 | −1277 | 689 | | | 70 | A | C |
| ATOM | 1214 | C | ASP A | 197 | 48.802 | 11.261 | 25.169 | | | 1.00 | 90.90 | | A | C |
| ANISOU | 1214 | C | ASP A | 197 | 14415 | 12672 | 7453 | −1594 | 525 | | | −11 | A | C |
| ATOM | 1215 | O | ASP A | 197 | 48.024 | 10.432 | 25.660 | | | 1.00 | 89.37 | | A | O |
| ANISOU | 1215 | O | ASP A | 197 | 14390 | 12357 | 7211 | −1743 | 373 | | | −134 | A | O |
| ATOM | 1216 | CB | ASP A | 197 | 50.994 | 10.773 | 26.315 | | | 1.00 | 107.68 | | A | C |
| ANISOU | 1216 | CB | ASP A | 197 | 16426 | 14476 | 10011 | −1046 | 952 | | | −130 | A | C |
| ATOM | 1217 | CG | ASP A | 197 | 51.927 | 10.465 | 25.138 | | | 1.00 | 111.71 | | A | C |
| ANISOU | 1217 | CG | ASP A | 197 | 17172 | 15012 | 10262 | −919 | 1260 | | | −203 | A | C |
| ATOM | 1218 | OD1 | ASP A | 197 | 51.861 | 11.164 | 24.100 | | | 1.00 | 114.07 | | A | O |
| ANISOU | 1218 | OD1 | ASP A | 197 | 17447 | 15542 | 10354 | −1007 | 1273 | | | −83 | A | O |
| ATOM | 1219 | OD2 | ASP A | 197 | 52.732 | 9.514 | 25.268 | | | 1.00 | 111.30 | | A | O1− |
| ANISOU | 1219 | OD2 | ASP A | 197 | 17321 | 14748 | 10219 | −723 | 1501 | | | −371 | A | O1− |
| ATOM | 1220 | N | ASN A | 198 | 48.661 | 11.749 | 23.941 | | | 1.00 | 94.34 | | A | N |
| ANISOU | 1220 | N | ASN A | 198 | 14894 | 13319 | 7630 | −1715 | 525 | | | 91 | A | N |
| ATOM | 1221 | CA | ASN A | 198 | 47.660 | 11.256 | 23.000 | | | 1.00 | 100.27 | | A | C |
| ANISOU | 1221 | CA | ASN A | 198 | 15803 | 14110 | 8184 | −1945 | 348 | | | 40 | A | C |
| ATOM | 1222 | C | ASN A | 198 | 46.232 | 11.537 | 23.475 | | | 1.00 | 87.84 | | A | C |
| ANISOU | 1222 | C | ASN A | 198 | 14031 | 12593 | 6754 | −2174 | 8 | | | 176 | A | C |
| ATOM | 1223 | O | ASN A | 198 | 45.317 | 10.764 | 23.185 | | | 1.00 | 97.24 | | A | O |
| ANISOU | 1223 | O | ASN A | 198 | 15376 | 13719 | 7850 | −2370 | −149 | | | 95 | A | O |
| ATOM | 1224 | CB | ASN A | 198 | 47.843 | 9.753 | 22.703 | | | 1.00 | 108.71 | | A | C |
| ANISOU | 1224 | CB | ASN A | 198 | 17283 | 14943 | 9078 | −1933 | 466 | | | −265 | A | C |
| ATOM | 1225 | CG | ASN A | 198 | 49.178 | 9.425 | 21.981 | | | 1.00 | 105.86 | | A | C |
| ANISOU | 1225 | CG | ASN A | 198 | 17131 | 14541 | 8550 | −1703 | 831 | | | −402 | A | C |
| ATOM | 1226 | OD1 | ASN A | 198 | 49.709 | 10.232 | 21.206 | | | 1.00 | 100.80 | | A | O |
| ANISOU | 1226 | OD1 | ASN A | 198 | 16376 | 14108 | 7815 | −1639 | 951 | | | −264 | A | O |
| ATOM | 1227 | ND2 | ASN A | 198 | 49.707 | 8.224 | 22.238 | | | 1.00 | 103.79 | | A | N |
| ANISOU | 1227 | ND2 | ASN A | 198 | 17160 | 14004 | 8270 | −1573 | 1012 | | | −657 | A | N |
| ATOM | 1228 | N | ASP A | 199 | 46.023 | 12.624 | 24.219 | | | 1.00 | 66.96 | | A | N |
| ANISOU | 1228 | N | ASP A | 199 | 11035 | 10061 | 4346 | −2151 | −97 | | | 393 | A | N |
| ATOM | 1229 | CA | ASP A | 199 | 44.699 | 13.227 | 24.319 | | | 1.00 | 59.25 | | A | C |
| ANISOU | 1229 | CA | ASP A | 199 | 9804 | 9212 | 3496 | −2349 | −384 | | | 597 | A | C |
| ATOM | 1230 | C | ASP A | 199 | 44.310 | 13.903 | 22.992 | | | 1.00 | 70.77 | | A | C |
| ANISOU | 1230 | C | ASP A | 199 | 11185 | 10881 | 4823 | −2492 | −462 | | | 784 | A | C |
| ATOM | 1231 | O | ASP A | 199 | 45.166 | 14.368 | 22.235 | | | 1.00 | 59.16 | | A | O |
| ANISOU | 1231 | O | ASP A | 199 | 9728 | 9508 | 3241 | −2397 | −302 | | | 835 | A | O |
| ATOM | 1232 | CB | ASP A | 199 | 44.672 | 14.255 | 25.452 | | | 1.00 | 58.12 | | A | C |
| ANISOU | 1232 | CB | ASP A | 199 | 9309 | 9126 | 3649 | −2257 | −444 | | | 777 | A | C |
| ATOM | 1233 | CG | ASP A | 199 | 44.193 | 13.672 | 26.788 | | | 1.00 | 75.22 | | A | C |
| ANISOU | 1233 | CG | ASP A | 199 | 11460 | 11147 | 5972 | −2258 | −534 | | | 669 | A | C |
| ATOM | 1234 | OD1 | ASP A | 199 | 43.886 | 12.465 | 26.837 | | | 1.00 | 82.68 | | A | O |
| ANISOU | 1234 | OD1 | ASP A | 199 | 12655 | 11940 | 6822 | −2331 | −556 | | | 475 | A | O |
| ATOM | 1235 | OD2 | ASP A | 199 | 44.121 | 14.425 | 27.793 | | | 1.00 | 68.22 | | A | O1− |
| ANISOU | 1235 | OD2 | ASP A | 199 | 10245 | 10238 | 5439 | −2126 | −564 | | | 761 | A | O1− |
| ATOM | 1236 | N | ASN A | 200 | 43.001 | 13.915 | 22.690 | | | 1.00 | 73.30 | | A | N |
| ANISOU | 1236 | N | ASN A | 200 | 11420 | 11276 | 5153 | −2732 | −704 | | | 901 | A | N |
| ATOM | 1237 | CA | ASN A | 200 | 42.438 | 14.644 | 21.553 | | | 1.00 | 74.06 | | A | C |
| ANISOU | 1237 | CA | ASN A | 200 | 11383 | 11583 | 5173 | −2905 | −827 | | | 1128 | A | C |
| ATOM | 1238 | C | ASN A | 200 | 41.486 | 15.718 | 22.053 | | | 1.00 | 66.84 | | A | C |
| ANISOU | 1238 | C | ASN A | 200 | 10053 | 10794 | 4548 | −2973 | −1031 | | | 1429 | A | C |
| ATOM | 1239 | O | ASN A | 200 | 40.981 | 15.651 | 23.178 | | | 1.00 | 65.28 | | A | O |
| ANISOU | 1239 | O | ASN A | 200 | 9725 | 10522 | 4558 | −2945 | −1109 | | | 1431 | A | O |
| ATOM | 1240 | CB | ASN A | 200 | 41.635 | 13.755 | 20.587 | | | 1.00 | 67.94 | | A | C |
| ANISOU | 1240 | CB | ASN A | 200 | 10855 | 10810 | 4150 | −3174 | −943 | | | 1052 | A | C |
| ATOM | 1241 | CG | ASN A | 200 | 42.471 | 12.708 | 19.924 | | | 1.00 | 66.46 | | A | C |
| ANISOU | 1241 | CG | ASN A | 200 | 11090 | 10504 | 3659 | −3124 | −745 | | | 765 | A | C |
| ATOM | 1242 | OD1 | ASN A | 200 | 42.072 | 11.552 | 19.834 | | | 1.00 | 67.77 | | A | O |
| ANISOU | 1242 | OD1 | ASN A | 200 | 11535 | 10522 | 3691 | −3252 | −783 | | | 578 | A | O |
| ATOM | 1243 | ND2 | ASN A | 200 | 43.648 | 13.096 | 19.467 | | | 1.00 | 66.40 | | A | N |
| ANISOU | 1243 | ND2 | ASN A | 200 | 11126 | 10553 | 3549 | −2932 | −520 | | | 739 | A | N |
| ATOM | 1244 | N | VAL A | 201 | 41.194 | 16.680 | 21.181 | | | 1.00 | 68.94 | | A | N |
| ANISOU | 1244 | N | VAL A | 201 | 6377 | 15233 | 4583 | −2088 | 787 | | | 548 | A | N |
| ATOM | 1245 | CA | VAL A | 201 | 40.238 | 17.711 | 21.546 | | | 1.00 | 67.74 | | A | C |
| ANISOU | 1245 | CA | VAL A | 201 | 6240 | 15038 | 4458 | −2096 | 678 | | | 889 | A | C |
| ATOM | 1246 | C | VAL A | 201 | 38.830 | 17.136 | 21.602 | | | 1.00 | 67.39 | | A | C |
| ANISOU | 1246 | C | VAL A | 201 | 6265 | 15073 | 4265 | −2155 | 531 | | | 715 | A | C |
| ATOM | 1247 | O | VAL A | 201 | 38.038 | 17.456 | 22.500 | | | 1.00 | 67.52 | | A | O |
| ANISOU | 1247 | O | VAL A | 201 | 6362 | 14834 | 4456 | −2173 | 398 | | | 818 | A | O |
| ATOM | 1248 | CB | VAL A | 201 | 40.334 | 18.891 | 20.569 | | | 1.00 | 69.85 | | A | C |
| ANISOU | 1248 | CB | VAL A | 201 | 6402 | 15540 | 4597 | −2030 | 731 | | | 1244 | A | C |
| ATOM | 1249 | CG1 | VAL A | 201 | 39.276 | 19.921 | 20.921 | | | 1.00 | 68.77 | | A | C |
| ANISOU | 1249 | CG1 | VAL A | 201 | 6297 | 15277 | 4554 | −1998 | 583 | | | 1552 | A | C |
| ATOM | 1250 | CG2 | VAL A | 201 | 41.753 | 19.478 | 20.621 | | | 1.00 | 70.14 | | A | C |
| ANISOU | 1250 | CG2 | VAL A | 201 | 6374 | 15461 | 4814 | −1994 | 897 | | | 1400 | A | C |
| ATOM | 1251 | N | THR A | 202 | 38.489 | 16.281 | 20.647 | | | 1.00 | 69.62 | | A | N |
| ANISOU | 1251 | N | THR A | 202 | 6509 | 15687 | 4256 | −2182 | 550 | | | 429 | A | N |
| ATOM | 1252 | CA | THR A | 202 | 37.164 | 15.671 | 20.663 | | | 1.00 | 69.57 | | A | C |
| ANISOU | 1252 | CA | THR A | 202 | 6547 | 15721 | 4165 | −2239 | 417 | | | 207 | A | C |
| ATOM | 1253 | C | THR A | 202 | 36.978 | 14.797 | 21.895 | | | 1.00 | 67.08 | | A | C |
| ANISOU | 1253 | C | THR A | 202 | 6396 | 15004 | 4088 | −2305 | 366 | | | −62 | A | C |
| ATOM | 1254 | O | THR A | 202 | 35.919 | 14.816 | 22.530 | | | 1.00 | 71.06 | | A | O |
| ANISOU | 1254 | O | THR A | 202 | 6974 | 15360 | 4667 | −2354 | 241 | | | −70 | A | O |
| ATOM | 1255 | CB | THR A | 202 | 36.952 | 14.836 | 19.406 | | | 1.00 | 72.63 | | A | C |
| ANISOU | 1255 | CB | THR A | 202 | 6843 | 16436 | 4319 | −2237 | 448 | | | −95 | A | C |
| ATOM | 1256 | OG1 | THR A | 202 | 37.362 | 15.594 | 18.266 | | | 1.00 | 78.79 | | A | O |
| ANISOU | 1256 | OG1 | THR A | 202 | 7490 | 17493 | 4954 | −2141 | 502 | | | 142 | A | O |
| ATOM | 1257 | CG2 | THR A | 202 | 35.500 | 14.439 | 19.286 | | | 1.00 | 73.02 | | A | C |
| ANISOU | 1257 | CG2 | THR A | 202 | 6885 | 16550 | 4308 | −2283 | 325 | | | −270 | A | C |
| ATOM | 1258 | N | LEU A | 203 | 38.001 | 14.018 | 22.241 | | | 1.00 | 66.72 | | A | N |
| ANISOU | 1258 | N | LEU A | 203 | 6413 | 14711 | 4226 | −2280 | 451 | | | −291 | A | N |
| ATOM | 1259 | CA | LEU A | 203 | 37.918 | 13.096 | 23.367 | | | 1.00 | 68.17 | | A | C |
| ANISOU | 1259 | CA | LEU A | 203 | 6775 | 14434 | 4694 | −2298 | 410 | | | −562 | A | C |
| ATOM | 1260 | C | LEU A | 203 | 37.710 | 13.851 | 24.678 | | | 1.00 | 67.47 | | A | C |
| ANISOU | 1260 | C | LEU A | 203 | 6781 | 13919 | 4935 | −2267 | 302 | | | −299 | A | C |
| ATOM | 1261 | O | LEU A | 203 | 36.740 | 13.613 | 25.417 | | | 1.00 | 60.16 | | A | O |
| ANISOU | 1261 | O | LEU A | 203 | 5969 | 12777 | 4112 | −2320 | 201 | | | −371 | A | O |
| ATOM | 1262 | CB | LEU A | 203 | 39.199 | 12.266 | 23.416 | | | 1.00 | 75.30 | | A | C |
| ANISOU | 1262 | CB | LEU A | 203 | 7715 | 15184 | 5713 | −2236 | 528 | | | −808 | A | C |
| ATOM | 1263 | CG | LEU A | 203 | 39.373 | 11.275 | 24.557 | | | 1.00 | 86.53 | | A | C |
| ANISOU | 1263 | CG | LEU A | 203 | 9338 | 16113 | 7427 | −2209 | 516 | | | −1081 | A | C |
| ATOM | 1264 | CD1 | LEU A | 203 | 38.193 | 10.300 | 24.596 | | | 1.00 | 91.62 | | A | C |
| ANISOU | 1264 | CD1 | LEU A | 203 | 10088 | 16741 | 7983 | −2318 | 484 | | | −1394 | A | C |
| ATOM | 1265 | CD2 | LEU A | 203 | 40.713 | 10.557 | 24.377 | | | 1.00 | 91.41 | | A | C |
| ANISOU | 1265 | CD2 | LEU A | 203 | 9958 | 16675 | 8101 | −2124 | 639 | | | −1302 | A | C |
| ATOM | 1266 | N | VAL A | 204 | 38.641 | 14.757 | 24.985 | | | 1.00 | 60.85 | | A | N |
| ANISOU | 1266 | N | VAL A | 204 | 5891 | 12955 | 4274 | −2185 | 332 | | | −10 | A | N |
| ATOM | 1267 | CA | VAL A | 204 | 38.563 | 15.562 | 26.200 | | | 1.00 | 58.10 | | A | C |
| ANISOU | 1267 | CA | VAL A | 204 | 5608 | 12223 | 4247 | −2147 | 237 | | | 247 | A | C |
| ATOM | 1268 | C | VAL A | 204 | 37.214 | 16.262 | 26.276 | | | 1.00 | 57.43 | | A | C |
| ANISOU | 1268 | C | VAL A | 204 | 5515 | 12228 | 4076 | −2210 | 112 | | | 460 | A | C |
| ATOM | 1269 | O | VAL A | 204 | 36.503 | 16.194 | 27.293 | | | 1.00 | 63.08 | | A | O |
| ANISOU | 1269 | O | VAL A | 204 | 6351 | 12632 | 4984 | −2234 | 1 | | | 446 | A | O |
| ATOM | 1270 | CB | VAL A | 204 | 39.730 | 16.570 | 26.248 | | | 1.00 | 57.99 | | A | C |
| ANISOU | 1270 | CB | VAL A | 204 | 5486 | 12166 | 4383 | −2067 | 311 | | | 534 | A | C |
| ATOM | 1271 | CG1 | VAL A | 204 | 39.459 | 17.645 | 27.289 | | | 1.00 | 55.56 | | A | C |
| ANISOU | 1271 | CG1 | VAL A | 204 | 5200 | 11560 | 4350 | −2044 | 212 | | | 853 | A | C |
| ATOM | 1272 | CG2 | VAL A | 204 | 41.051 | 15.850 | 26.507 | | | 1.00 | 58.13 | | A | C |
| ANISOU | 1272 | CG2 | VAL A | 204 | 5527 | 11999 | 4561 | −1987 | 405 | | | 295 | A | C |
| ATOM | 1273 | N | SER A | 205 | 36.834 | 16.931 | 25.184 | | | 1.00 | 59.39 | | A | N |
| ANISOU | 1273 | N | SER A | 205 | 5626 | 12916 | 4022 | −2228 | 131 | | | 655 | A | N |
| ATOM | 1274 | CA | SER A | 205 | 35.606 | 17.716 | 25.205 | | | 1.00 | 58.96 | | A | C |
| ANISOU | 1274 | CA | SER A | 205 | 5549 | 12978 | 3874 | −2261 | 10 | | | 887 | A | C |
| ATOM | 1275 | C | SER A | 205 | 34.384 | 16.836 | 25.420 | | | 1.00 | 66.98 | | A | C |
| ANISOU | 1275 | C | SER A | 205 | 6650 | 13986 | 4814 | −2350 | −88 | | | 586 | A | C |
| ATOM | 1276 | O | SER A | 205 | 33.416 | 17.266 | 26.057 | | | 1.00 | 72.08 | | A | O |
| ANISOU | 1276 | O | SER A | 205 | 7339 | 14501 | 5548 | −2382 | −214 | | | 702 | A | O |
| ATOM | 1277 | CB | SER A | 205 | 35.457 | 18.538 | 23.918 | | | 1.00 | 61.54 | | A | C |
| ANISOU | 1277 | CB | SER A | 205 | 5722 | 13807 | 3852 | −2233 | 59 | | | 1142 | A | C |
| ATOM | 1278 | OG | SER A | 205 | 35.619 | 17.748 | 22.752 | | | 1.00 | 64.29 | | A | O |
| ANISOU | 1278 | OG | SER A | 205 | 6000 | 14513 | 3913 | −2238 | 142 | | | 878 | A | O |
| ATOM | 1279 | N | SER A | 206 | 34.407 | 15.602 | 24.920 | | | 1.00 | 70.00 | | A | N |
| ANISOU | 1279 | N | SER A | 206 | 7054 | 14496 | 5049 | −2399 | −25 | | | 186 | A | N |
| ATOM | 1280 | CA | SER A | 206 | 33.249 | 14.742 | 25.127 | | | 1.00 | 66.46 | | A | C |
| ANISOU | 1280 | CA | SER A | 206 | 6681 | 14025 | 4547 | −2501 | −92 | | | −129 | A | C |
| ATOM | 1281 | C | SER A | 206 | 33.204 | 14.216 | 26.560 | | | 1.00 | 57.36 | | A | C |
| ANISOU | 1281 | C | SER A | 206 | 5726 | 12306 | 3762 | −2519 | −129 | | | −258 | A | C |
| ATOM | 1282 | O | SER A | 206 | 32.124 | 14.138 | 27.153 | | | 1.00 | 56.23 | | A | O |
| ANISOU | 1282 | O | SER A | 206 | 5654 | 12026 | 3684 | −2592 | −223 | | | −313 | A | O |
| ATOM | 1283 | CB | SER A | 206 | 33.267 | 13.596 | 24.120 | | | 1.00 | 62.73 | | A | C |
| ANISOU | 1283 | CB | SER A | 206 | 6162 | 13867 | 3805 | −2555 | 3 | | | −529 | A | C |
| ATOM | 1284 | OG | SER A | 206 | 34.365 | 12.747 | 24.396 | | | 1.00 | 71.83 | | A | C |
| ANISOU | 1284 | OG | SER A | 206 | 7405 | 14763 | 5124 | −2522 | 116 | | | −746 | A | C |
| ATOM | 1285 | N | LYS A | 207 | 34.361 | 13.855 | 27.133 | | | 1.00 | 62.27 | | A | N |
| ANISOU | 1285 | N | LYS A | 207 | 6437 | 12602 | 4619 | −2444 | −54 | | | −311 | A | N |
| ATOM | 1286 | CA | LYS A | 207 | 34.414 | 13.498 | 28.554 | | | 1.00 | 61.05 | | A | C |
| ANISOU | 1286 | CA | LYS A | 207 | 6478 | 11905 | 4815 | −2420 | −93 | | | −376 | A | C |
| ATOM | 1287 | C | LYS A | 207 | 33.789 | 14.580 | 29.418 | | | 1.00 | 54.39 | | A | C |
| ANISOU | 1287 | C | LYS A | 207 | 5646 | 10871 | 4147 | −2415 | −228 | | | −47 | A | C |
| ATOM | 1288 | O | LYS A | 207 | 32.980 | 14.295 | 30.304 | | | 1.00 | 57.02 | | A | O |
| ANISOU | 1288 | O | LYS A | 207 | 6113 | 10925 | 4626 | −2466 | −298 | | | −132 | A | O |
| ATOM | 1289 | CB | LYS A | 207 | 35.861 | 13.255 | 29.006 | | | 1.00 | 56.97 | | A | C |
| ANISOU | 1289 | CB | LYS A | 207 | 6016 | 11120 | 4510 | −2295 | −12 | | | −401 | A | C |
| ATOM | 1290 | CG | LYS A | 207 | 36.315 | 11.831 | 28.819 | | | 1.00 | 66.33 | | A | C |
| ANISOU | 1290 | CG | LYS A | 207 | 7305 | 12235 | 5664 | −2291 | 102 | | | −816 | A | C |
| ATOM | 1291 | CD | LYS A | 207 | 37.805 | 11.688 | 28.909 | | | 1.00 | 74.49 | | A | C |
| ANISOU | 1291 | CD | LYS A | 207 | 8331 | 13150 | 6820 | −2156 | 186 | | | −835 | A | C |
| ATOM | 1292 | CE | LYS A | 207 | 38.233 | 10.435 | 28.141 | | | 1.00 | 83.59 | | A | C |
| ANISOU | 1292 | CE | LYS A | 207 | 9504 | 14448 | 7810 | −2167 | 316 | | | −1220 | A | C |
| ATOM | 1293 | NZ | LYS A | 207 | 39.693 | 10.148 | 28.252 | | | 1.00 | 87.90 | | A | N1+ |
| ANISOU | 1293 | NZ | LYS A | 207 | 10052 | 14869 | 8477 | −2026 | 399 | | | −1293 | A | N1+ |
| ATOM | 1294 | N | PHE A | 208 | 34.152 | 15.830 | 29.172 | | | 1.00 | 51.45 | | A | N |
| ANISOU | 1294 | N | PHE A | 208 | 5138 | 10641 | 3768 | −2358 | −253 | | | 329 | A | N |
| ATOM | 1295 | CA | PHE A | 208 | 33.703 | 16.910 | 30.035 | | | 1.00 | 52.14 | | A | C |
| ANISOU | 1295 | CA | PHE A | 208 | 5234 | 10520 | 4055 | −2341 | −371 | | | 653 | A | C |
| ATOM | 1296 | C | PHE A | 208 | 32.404 | 17.562 | 29.583 | | | 1.00 | 56.75 | | A | C |
| ANISOU | 1296 | C | PHE A | 208 | 5734 | 11392 | 4435 | −2412 | −471 | | | 808 | A | C |
| ATOM | 1297 | O | PHE A | 208 | 31.812 | 18.318 | 30.356 | | | 1.00 | 51.09 | | A | O |
| ANISOU | 1297 | O | PHE A | 208 | 5041 | 10489 | 3880 | −2415 | −582 | | | 1026 | A | O |
| ATOM | 1298 | CB | PHE A | 208 | 34.798 | 17.966 | 30.152 | | | 1.00 | 56.47 | | A | C |
| ANISOU | 1298 | CB | PHE A | 208 | 5689 | 11009 | 4758 | −2241 | −334 | | | 979 | A | C |
| ATOM | 1299 | CG | PHE A | 208 | 35.953 | 17.524 | 31.004 | | | 1.00 | 59.44 | | A | C |
| ANISOU | 1299 | CG | PHE A | 208 | 6154 | 11010 | 5421 | −2150 | −285 | | | 861 | A | C |
| ATOM | 1300 | CD1 | PHE A | 208 | 36.950 | 16.711 | 30.484 | | | 1.00 | 66.07 | | A | C |
| ANISOU | 1300 | CD1 | PHE A | 208 | 6986 | 11926 | 6191 | −2107 | −161 | | | 625 | A | C |
| ATOM | 1301 | CD2 | PHE A | 208 | 36.010 | 17.868 | 32.347 | | | 1.00 | 55.08 | | A | C |
| ANISOU | 1301 | CD2 | PHE A | 208 | 5694 | 10034 | 5199 | −2096 | −368 | | | 962 | A | C |
| ATOM | 1302 | CE1 | PHE A | 208 | 38.008 | 16.280 | 31.284 | | | 1.00 | 57.89 | | A | C |
| ANISOU | 1302 | CE1 | PHE A | 208 | 6031 | 10556 | 5409 | −1999 | −125 | | | 499 | A | C |
| ATOM | 1303 | CE2 | PHE A | 208 | 37.058 | 17.448 | 33.146 | | | 1.00 | 51.66 | | A | C |
| ANISOU | 1303 | CE2 | PHE A | 208 | 5340 | 9278 | 5012 | −1985 | −333 | | | 839 | A | C |
| ATOM | 1304 | CZ | PHE A | 208 | 38.061 | 16.649 | 32.614 | | | 1.00 | 45.61 | | A | C |
| ANISOU | 1304 | CZ | PHE A | 208 | 4564 | 8596 | 4171 | −1932 | −213 | | | 606 | A | C |
| ATOM | 1305 | N | GLY A | 209 | 31.901 | 17.174 | 28.418 | | | 1.00 | 66.58 | | A | N |
| ANISOU | 1305 | N | GLY A | 209 | 6878 | 13093 | 5326 | −2460 | −441 | | | 690 | A | N |
| ATOM | 1306 | CA | GLY A | 209 | 30.677 | 17.751 | 27.897 | | | 1.00 | 59.09 | | A | C |
| ANISOU | 1306 | CA | GLY A | 209 | 5836 | 12463 | 4151 | −2502 | −543 | | | 819 | A | C |
| ATOM | 1307 | C | GLY A | 209 | 30.813 | 19.235 | 27.648 | | | 1.00 | 55.88 | | A | C |
| ANISOU | 1307 | C | GLY A | 209 | 5316 | 12148 | 3769 | −2404 | −562 | | | 1280 | A | C |
| ATOM | 1308 | O | GLY A | 209 | 29.890 | 20.013 | 27.871 | | | 1.00 | 52.05 | | A | O |
| ANISOU | 1308 | O | GLY A | 209 | 4817 | 11516 | 3445 | −2374 | −645 | | | 1461 | A | O |
| ATOM | 1309 | N | ALA A | 210 | 31.993 | 19.620 | 27.184 | | | 1.00 | 64.40 | | A | N |
| ANISOU | 1309 | N | ALA A | 210 | 6311 | 13395 | 4764 | −2333 | −450 | | | 1442 | A | N |
| ATOM | 1310 | CA | ALA A | 210 | 32.298 | 21.003 | 26.868 | | | 1.00 | 58.70 | | A | C |
| ANISOU | 1310 | CA | ALA A | 210 | 5501 | 12602 | 4201 | −2223 | −435 | | | 1846 | A | C |
| ATOM | 1311 | C | ALA A | 210 | 32.527 | 21.082 | 25.371 | | | 1.00 | 57.43 | | A | C |
| ANISOU | 1311 | C | ALA A | 210 | 5196 | 12779 | 3847 | −2122 | −379 | | | 1958 | A | C |
| ATOM | 1312 | O | ALA A | 210 | 33.151 | 20.198 | 24.790 | | | 1.00 | 57.64 | | A | O |
| ANISOU | 1312 | O | ALA A | 210 | 5164 | 12735 | 4001 | −2020 | −390 | | | 2258 | A | O |
| ATOM | 1313 | CB | ALA A | 210 | 33.532 | 21.465 | 27.623 | | | 1.00 | 58.08 | | A | C |
| ANISOU | 1313 | CB | ALA A | 210 | 5447 | 12330 | 4290 | −2206 | −333 | | | 1985 | A | C |
| ATOM | 1314 | N | SER A | 211 | 32.001 | 22.122 | 24.739 | | | 1.00 | 59.87 | | A | N |
| ANISOU | 1314 | N | SER A | 211 | 5453 | 13432 | 3861 | −2139 | −322 | | | 1706 | A | N |
| ATOM | 1315 | CA | SER A | 211 | 32.158 | 22.270 | 23.300 | | | 1.00 | 76.19 | | A | C |
| ANISOU | 1315 | CA | SER A | 211 | 7393 | 15848 | 5708 | −2037 | −255 | | | 1787 | A | C |
| ATOM | 1316 | C | SER A | 211 | 33.620 | 22.433 | 22.910 | | | 1.00 | 80.59 | | A | C |
| ANISOU | 1316 | C | SER A | 211 | 7920 | 16415 | 6287 | −1973 | −123 | | | 1979 | A | C |
| ATOM | 1317 | O | SER A | 211 | 34.369 | 23.155 | 23.565 | | | 1.00 | 79.45 | | A | O |
| ANISOU | 1317 | O | SER A | 211 | 7814 | 15975 | 6397 | −1957 | −101 | | | 2200 | A | O |
| ATOM | 1318 | CB | SER A | 211 | 31.374 | 23.487 | 22.809 | | | 1.00 | 87.70 | | A | C |
| ANISOU | 1318 | CB | SER A | 211 | 8782 | 17365 | 7174 | −1931 | −325 | | | 2014 | A | C |
| ATOM | 1319 | OG | SER A | 211 | 31.911 | 24.684 | 23.350 | | | 1.00 | 86.65 | | A | O |
| ANISOU | 1319 | OG | SER A | 211 | 8548 | 17571 | 6805 | −1820 | −253 | | | 2092 | A | O |
| ATOM | 1320 | N | GLN A | 212 | 34.018 | 21.774 | 21.825 | | | 1.00 | 94.03 | | A | N |
| ANISOU | 1320 | N | GLN A | 212 | 9542 | 18451 | 7732 | −1932 | −28 | | | 1886 | A | N |
| ATOM | 1321 | CA | GLN A | 212 | 35.389 | 21.870 | 21.334 | | | 1.00 | 73.86 | | A | C |
| ANISOU | 1321 | CA | GLN A | 212 | 6963 | 15934 | 5166 | −1899 | 126 | | | 1967 | A | C |
| ATOM | 1322 | C | GLN A | 212 | 35.714 | 23.297 | 20.906 | | | 1.00 | 68.66 | | A | C |
| ANISOU | 1322 | C | GLN A | 212 | 6280 | 15163 | 4645 | −1784 | 174 | | | 2363 | A | C |
| ATOM | 1323 | O | GLN A | 212 | 36.815 | 23.783 | 21.131 | | | 1.00 | 68.36 | | A | O |
| ANISOU | 1323 | O | GLN A | 212 | 6250 | 14967 | 4756 | −1776 | 291 | | | 2483 | A | O |
| ATOM | 1324 | CB | GLN A | 212 | 35.668 | 20.862 | 20.216 | | | 1.00 | 71.09 | | A | C |
| ANISOU | 1324 | CB | GLN A | 212 | 6538 | 15973 | 4501 | −1891 | 204 | | | 1720 | A | C |
| ATOM | 1325 | CG | GLN A | 212 | 34.441 | 20.368 | 19.476 | | | 1.00 | 70.66 | | A | C |
| ANISOU | 1325 | CG | GLN A | 212 | 6512 | 15998 | 4338 | −2000 | 146 | | | 1295 | A | C |
| ATOM | 1326 | CD | GLN A | 212 | 34.676 | 19.025 | 18.811 | | | 1.00 | 78.81 | | A | C |
| ANISOU | 1326 | CD | GLN A | 212 | 7458 | 17398 | 5090 | −1994 | 207 | | | 1005 | A | C |
| ATOM | 1327 | OE1 | GLN A | 212 | 35.773 | 18.474 | 18.874 | | | 1.00 | 87.67 | | A | O |
| ANISOU | 1327 | OE1 | GLN A | 212 | 8576 | 18575 | 6161 | −2020 | 324 | | | 837 | A | O |
| ATOM | 1328 | NE2 | GLN A | 212 | 33.642 | 18.489 | 18.174 | | | 1.00 | 75.62 | | A | N |
| ANISOU | 1328 | NE2 | GLN A | 212 | 6974 | 17243 | 4515 | −1958 | 131 | | | 923 | A | N |
| ATOM | 1329 | N | VAL A | 213 | 34.753 | 23.964 | 20.283 | | | 1.00 | 74.66 | | A | N |
| ANISOU | 1329 | N | VAL A | 213 | 7016 | 15968 | 5385 | −1696 | 96 | | | 2550 | A | N |
| ATOM | 1330 | CA | VAL A | 213 | 34.957 | 25.337 | 19.862 | | | 1.00 | 70.77 | | A | C |
| ANISOU | 1330 | CA | VAL A | 213 | 6512 | 15397 | 4982 | −1574 | 167 | | | 2895 | A | C |
| ATOM | 1331 | C | VAL A | 213 | 35.206 | 26.206 | 21.088 | | | 1.00 | 68.00 | | A | C |
| ANISOU | 1331 | C | VAL A | 213 | 6220 | 14608 | 5008 | −1573 | 130 | | | 3102 | A | C |
| ATOM | 1332 | O | VAL A | 213 | 36.042 | 27.103 | 21.053 | | | 1.00 | 68.58 | | A | O |
| ANISOU | 1332 | O | VAL A | 213 | 6302 | 14520 | 5234 | −1504 | 230 | | | 3338 | A | O |
| ATOM | 1333 | CB | VAL A | 213 | 33.751 | 25.877 | 19.078 | | | 1.00 | 73.33 | | A | C |
| ANISOU | 1333 | CB | VAL A | 213 | 6786 | 15984 | 5092 | −1460 | 119 | | | 2982 | A | C |
| ATOM | 1334 | CG1 | VAL A | 213 | 33.995 | 27.318 | 18.658 | | | 1.00 | 74.75 | | A | C |
| ANISOU | 1334 | CG1 | VAL A | 213 | 6982 | 16058 | 5362 | −1324 | 191 | | | 3337 | A | C |
| ATOM | 1335 | CG2 | VAL A | 213 | 33.479 | 25.001 | 17.868 | | | 1.00 | 76.22 | | A | C |
| ANISOU | 1335 | CG2 | VAL A | 213 | 7085 | 16763 | 5113 | −1452 | 175 | | | 2773 | A | C |
| ATOM | 1336 | N | GLU A | 214 | 34.473 | 25.951 | 22.168 | | | 1.00 | 68.86 | | A | N |
| ANISOU | 1336 | N | GLU A | 214 | 6373 | 14511 | 5281 | −1647 | −8 | | | 3006 | A | N |
| ATOM | 1337 | CA | GLU A | 214 | 34.675 | 26.729 | 23.386 | | | 1.00 | 65.76 | | A | C |
| ANISOU | 1337 | CA | GLU A | 214 | 6029 | 13698 | 5259 | −1638 | −57 | | | 3175 | A | C |
| ATOM | 1338 | C | GLU A | 214 | 36.075 | 26.501 | 23.938 | | | 1.00 | 64.27 | | A | C |
| ANISOU | 1338 | C | GLU A | 214 | 5869 | 13285 | 5266 | −1685 | 39 | | | 3157 | A | C |
| ATOM | 1339 | O | GLU A | 214 | 36.771 | 27.446 | 24.334 | | | 1.00 | 69.95 | | A | O |
| ANISOU | 1339 | O | GLU A | 214 | 6595 | 13743 | 6241 | −1628 | 99 | | | 3344 | A | O |
| ATOM | 1340 | CB | GLU A | 214 | 33.613 | 26.382 | 24.424 | | | 1.00 | 71.17 | | A | C |
| ANISOU | 1340 | CB | GLU A | 214 | 6753 | 14234 | 6053 | −1700 | −226 | | | 3070 | A | C |
| ATOM | 1341 | CG | GLU A | 214 | 32.237 | 26.915 | 24.072 | | | 1.00 | 85.00 | | A | C |
| ANISOU | 1341 | CG | GLU A | 214 | 8465 | 16141 | 7690 | −1633 | −303 | | | 3123 | A | C |
| ATOM | 1342 | CD | GLU A | 214 | 32.224 | 28.424 | 23.942 | | | 1.00 | 98.77 | | A | C |
| ANISOU | 1342 | CD | GLU A | 214 | 10194 | 17748 | 9586 | −1500 | −273 | | | 3417 | A | C |
| ATOM | 1343 | OE1 | GLU A | 214 | 33.167 | 29.069 | 24.445 | | | 1.00 | 98.79 | | A | O |
| ANISOU | 1343 | OE1 | GLU A | 214 | 10225 | 17456 | 9854 | −1475 | −224 | | | 3561 | A | O |
| ATOM | 1344 | OE2 | GLU A | 214 | 31.274 | 28.969 | 23.342 | | | 1.00 | 104.66 | | A | O1− |
| ANISOU | 1344 | OE2 | GLU A | 214 | 10904 | 18679 | 10181 | −1415 | −293 | | | 3485 | A | O1− |
| ATOM | 1345 | N | MET A | 215 | 36.509 | 25.245 | 23.910 | | | 1.00 | 61.93 | | A | N |
| ANISOU | 1345 | N | MET A | 215 | 5586 | 13093 | 4852 | −1784 | 72 | | | 2906 | A | N |
| ATOM | 1346 | CA | MET A | 215 | 37.833 | 24.905 | 24.389 | | | 1.00 | 60.37 | | A | C |
| ANISOU | 1346 | CA | MET A | 215 | 5382 | 12780 | 4775 | −1815 | 213 | | | 2856 | A | C |
| ATOM | 1347 | C | MET A | 215 | 38.867 | 25.617 | 23.547 | | | 1.00 | 62.68 | | A | C |
| ANISOU | 1347 | C | MET A | 215 | 5618 | 13111 | 5084 | −1731 | 373 | | | 3048 | A | C |
| ATOM | 1348 | O | MET A | 215 | 39.794 | 26.221 | 24.077 | | | 1.00 | 65.61 | | A | O |
| ANISOU | 1348 | O | MET A | 215 | 5993 | 13190 | 5747 | −1697 | 433 | | | 3195 | A | O |
| ATOM | 1349 | CB | MET A | 215 | 38.081 | 23.412 | 24.229 | | | 1.00 | 62.60 | | A | C |
| ANISOU | 1349 | CB | MET A | 215 | 5659 | 13308 | 4817 | −1905 | 277 | | | 2537 | A | C |
| ATOM | 1350 | CG | MET A | 215 | 37.535 | 22.534 | 25.330 | | | 1.00 | 66.60 | | A | C |
| ANISOU | 1350 | CG | MET A | 215 | 6249 | 13701 | 5354 | −2006 | 175 | | | 2318 | A | C |
| ATOM | 1351 | SD | MET A | 215 | 38.234 | 20.88 | 8 25.148 | | | 1.00 | 72.76 | | A | S |
| ANISOU | 1351 | SD | MET A | 215 | 7044 | 14664 | 5937 | −2078 | 282 | | | 1882 | A | S |
| ATOM | 1352 | CE | MET A | 215 | 39.973 | 21.248 | 25.348 | | | 1.00 | 67.92 | | A | C |
| ANISOU | 1352 | CE | MET A | 215 | 6385 | 13768 | 5652 | −2025 | 425 | | | 1881 | A | C |
| ATOM | 1353 | N | LEU A | 216 | 38.677 | 25.593 | 22.231 | | | 1.00 | 65.71 | | A | N |
| ANISOU | 1353 | N | LEU A | 216 | 5957 | 13852 | 5158 | −1695 | 447 | | | 3038 | A | N |
| ATOM | 1354 | CA | LEU A | 216 | 39.630 | 26.233 | 21.341 | | | 1.00 | 68.27 | | A | C |
| ANISOU | 1354 | CA | LEU A | 216 | 6241 | 14243 | 5455 | −1634 | 632 | | | 3185 | A | C |
| ATOM | 1355 | C | LEU A | 216 | 39.696 | 27.729 | 21.597 | | | 1.00 | 68.48 | | A | C |
| ANISOU | 1355 | C | LEU A | 216 | 6295 | 14024 | 5701 | −1543 | 651 | | | 3491 | A | C |
| ATOM | 1356 | O | LEU A | 216 | 40.772 | 28.314 | 21.598 | | | 1.00 | 72.05 | | A | O |
| ANISOU | 1356 | O | LEU A | 216 | 6740 | 14320 | 6317 | −1524 | 811 | | | 3605 | A | O |
| ATOM | 1357 | CB | LEU A | 216 | 39.279 | 25.961 | 19.879 | | | 1.00 | 80.18 | | A | C |
| ANISOU | 1357 | CB | LEU A | 216 | 7706 | 16187 | 6573 | −1601 | 684 | | | 3108 | A | C |
| ATOM | 1358 | CG | LEU A | 216 | 39.370 | 24.509 | 19.407 | | | 1.00 | 85.93 | | A | C |
| ANISOU | 1358 | CG | LEU A | 216 | 8411 | 17058 | 7182 | −1703 | 707 | | | 2767 | A | C |
| ATOM | 1359 | CD1 | LEU A | 216 | 38.850 | 24.36 | 7 17.985 | | | 1.00 | 78.33 | | A | C |
| ANISOU | 1359 | CD1 | LEU A | 216 | 7395 | 16514 | 5851 | −1690 | 758 | | | 2590 | A | C |
| ATOM | 1360 | CD2 | LEU A | 216 | 40.790 | 23.985 | 19.522 | | | 1.00 | 99.52 | | A | C |
| ANISOU | 1360 | CD2 | LEU A | 216 | 10111 | 18564 | 9138 | −1743 | 859 | | | 2736 | A | C |
| ATOM | 1361 | N | ALA A | 217 | 38.546 | 28.350 | 21.821 | | | 1.00 | 67.78 | | A | N |
| ANISOU | 1361 | N | ALA A | 217 | 6235 | 13882 | 5637 | −1492 | 502 | | | 3599 | A | N |
| ATOM | 1362 | CA | ALA A | 217 | 38.524 | 29.780 | 22.062 | | | 1.00 | 67.65 | | A | C |
| ANISOU | 1362 | CA | ALA A | 217 | 6251 | 13588 | 5864 | −1408 | 513 | | | 3849 | A | C |
| ATOM | 1363 | C | ALA A | 217 | 39.286 | 30.106 | 23.329 | | | 1.00 | 65.09 | | A | C |
| ANISOU | 1363 | C | ALA A | 217 | 5948 | 12855 | 5929 | −1444 | 534 | | | 3850 | A | C |
| ATOM | 1364 | O | ALA A | 217 | 40.053 | 31.061 | 23.376 | | | 1.00 | 65.90 | | A | O |
| ANISOU | 1364 | O | ALA A | 217 | 6060 | 12761 | 6219 | −1404 | 667 | | | 3995 | A | O |
| ATOM | 1365 | CB | ALA A | 217 | 37.092 | 30.272 | 22.170 | | | 1.00 | 66.83 | | A | C |
| ANISOU | 1365 | CB | ALA A | 217 | 6162 | 13476 | 5755 | −1357 | 335 | | | 3908 | A | C |
| ATOM | 1366 | N | GLU A | 218 | 39.055 | 29.308 | 24.363 | | | 1.00 | 62.20 | | A | N |
| ANISOU | 1366 | N | GLU A | 218 | 5594 | 12356 | 5684 | −1520 | 416 | | | 3673 | A | N |
| ATOM | 1367 | CA | GLU A | 218 | 39.727 | 29.504 | 25.642 | | | 1.00 | 63.47 | | A | C |
| ANISOU | 1367 | CA | GLU A | 218 | 5773 | 12121 | 6221 | −1532 | 412 | | | 3659 | A | C |
| ATOM | 1368 | C | GLU A | 218 | 41.237 | 29.262 | 25.640 | | | 1.00 | 75.47 | | A | C |
| ANISOU | 1368 | C | GLU A | 218 | 7246 | 13603 | 7827 | −1574 | 603 | | | 3574 | A | C |
| ATOM | 1369 | O | GLU A | 218 | 41.990 | 29.982 | 26.293 | | | 1.00 | 75.19 | | A | O |
| ANISOU | 1369 | O | GLU A | 218 | 7203 | 13262 | 8104 | −1557 | 664 | | | 3601 | A | O |
| ATOM | 1370 | CB | GLU A | 218 | 39.069 | 28.655 | 26.731 | | | 1.00 | 56.56 | | A | C |
| ANISOU | 1370 | CB | GLU A | 218 | 4941 | 11102 | 5447 | −1584 | 220 | | | 3509 | A | C |
| ATOM | 1371 | CG | GLU A | 218 | 39.656 | 28.867 | 28.116 | | | 1.00 | 61.42 | | A | C |
| ANISOU | 1371 | CG | GLU A | 218 | 5582 | 11321 | 6434 | −1580 | 189 | | | 3460 | A | C |
| ATOM | 1372 | CD | GLU A | 218 | 39.389 | 30.254 | 28.654 | | | 1.00 | 72.09 | | A | C |
| ANISOU | 1372 | CD | GLU A | 218 | 6947 | 12385 | 8057 | −1485 | 176 | | | 3620 | A | C |
| ATOM | 1373 | OE1 | GLU A | 218 | 40.314 | 30.863 | 29.225 | | | 1.00 | 80.76 | | A | O |
| ANISOU | 1373 | OE1 | GLU A | 218 | 8018 | 13325 | 9341 | −1464 | 320 | | | 3652 | A | O |
| ATOM | 1374 | OE2 | GLU A | 218 | 38.252 | 30.739 | 28.503 | | | 1.00 | 69.14 | | A | O1− |
| ANISOU | 1374 | OE2 | GLU A | 218 | 6602 | 11949 | 7718 | −1440 | 31 | | | 3686 | A | O1− |
| ATOM | 1375 | N | ASN A | 219 | 41.674 | 28.230 | 24.932 | | | 1.00 | 78.62 | | A | N |
| ANISOU | 1375 | N | ASN A | 219 | 7598 | 14314 | 7959 | −1625 | 705 | | | 3445 | A | N |
| ATOM | 1376 | CA | ASN A | 219 | 43.082 | 27.844 | 24.923 | | | 1.00 | 73.26 | | A | C |
| ANISOU | 1376 | CA | ASN A | 219 | 6849 | 13623 | 7362 | −1675 | 871 | | | 3295 | A | C |
| ATOM | 1377 | C | ASN A | 219 | 43.973 | 28.265 | 23.756 | | | 1.00 | 82.74 | | A | C |
| ANISOU | 1377 | C | ASN A | 219 | 7998 | 14985 | 8455 | −1657 | 1097 | | | 3372 | A | C |
| ATOM | 1378 | O | ASN A | 219 | 45.178 | 28.059 | 23.830 | | | 1.00 | 95.69 | | A | O |
| ANISOU | 1378 | O | ASN A | 219 | 9563 | 16580 | 10216 | −1694 | 1252 | | | 3253 | A | O |
| ATOM | 1379 | CB | ASN A | 219 | 43.181 | 26.326 | 25.058 | | | 1.00 | 64.39 | | A | C |
| ANISOU | 1379 | CB | ASN A | 219 | 5701 | 12720 | 6043 | −1753 | 841 | | | 3022 | A | C |
| ATOM | 1380 | CG | ASN A | 219 | 42.799 | 25.840 | 26.432 | | | 1.00 | 58.78 | | A | C |
| ANISOU | 1380 | CG | ASN A | 219 | 5035 | 11780 | 5517 | −1793 | 684 | | | 2897 | A | C |
| ATOM | 1381 | OD1 | ASN A | 219 | 43.374 | 26.260 | 27.428 | | | 1.00 | 60.24 | | A | O |
| ANISOU | 1381 | OD1 | ASN A | 219 | 5185 | 11706 | 5997 | −1799 | 711 | | | 2815 | A | O |
| ATOM | 1382 | ND2 | ASN A | 219 | 41.831 | 24.943 | 26.491 | | | 1.00 | 57.87 | | A | N |
| ANISOU | 1382 | ND2 | ASN A | 219 | 4992 | 11760 | 5234 | −1819 | 523 | | | 2867 | A | N |
| ATOM | 1383 | N | ASN A | 220 | 43.396 | 28.809 | 22.687 | | | 1.00 | 80.02 | | A | N |
| ANISOU | 1383 | N | ASN A | 220 | 7685 | 14828 | 7890 | −1598 | 1124 | | | 3552 | A | N |
| ATOM | 1384 | CA | ASN A | 220 | 44.159 | 29.235 | 21.499 | | | 1.00 | 82.61 | | A | C |
| ANISOU | 1384 | CA | ASN A | 220 | 7983 | 15376 | 8029 | −1579 | 1334 | | | 3621 | A | C |
| ATOM | 1385 | C | ASN A | 220 | 45.146 | 28.165 | 20.998 | | | 1.00 | 77.17 | | A | C |
| ANISOU | 1385 | C | ASN A | 220 | 7207 | 14868 | 7246 | −1648 | 1475 | | | 3383 | A | C |
| ATOM | 1386 | O | ASN A | 220 | 46.310 | 28.433 | 20.732 | | | 1.00 | 73.61 | | A | O |
| ANISOU | 1386 | O | ASN A | 220 | 6699 | 14304 | 6965 | −1674 | 1665 | | | 3359 | A | O |
| ATOM | 1387 | CB | ASN A | 220 | 44.693 | 30.693 | 21.538 | | | 1.00 | 88.14 | | A | C |
| ANISOU | 1387 | CB | ASN A | 220 | 8712 | 15823 | 8953 | −1535 | 1487 | | | 3852 | A | C |
| ATOM | 1388 | CG | ASN A | 220 | 45.834 | 30.908 | 22.513 | | | 1.00 | 90.15 | | A | C |
| ANISOU | 1388 | CG | ASN A | 220 | 8914 | 15757 | 9582 | −1590 | 1619 | | | 3771 | A | C |
| ATOM | 1389 | OD1 | ASN A | 220 | 46.845 | 30.211 | 22.479 | | | 1.00 | 92.93 | | A | O |
| ANISOU | 1389 | OD1 | ASN A | 220 | 9217 | 15997 | 10095 | −1637 | 1546 | | | 3576 | A | O |
| ATOM | 1390 | ND2 | ASN A | 220 | 45.683 | 31.902 | 23.378 | | | 1.00 | 84.96 | | A | N |
| ANISOU | 1390 | ND2 | ASN A | 220 | 8266 | 14952 | 9063 | −1582 | 1820 | | | 3909 | A | N |
| ATOM | 1391 | N | HIS A | 221 | 44.638 | 26.942 | 20.889 | | | 1.00 | 75.95 | | A | N |
| ANISOU | 1391 | N | HIS A | 221 | 7037 | 15017 | 6802 | −1676 | 1390 | | | 3185 | A | N |
| ATOM | 1392 | CA | HIS A | 221 | 45.401 | 25.776 | 20.453 | | | 1.00 | 72.96 | | A | C |
| ANISOU | 1392 | CA | HIS A | 221 | 6582 | 14812 | 6327 | −1740 | 1466 | | | 2884 | A | C |
| ATOM | 1393 | C | HIS A | 221 | 46.700 | 25.489 | 21.176 | | | 1.00 | 71.64 | | A | C |
| ANISOU | 1393 | C | HIS A | 221 | 6339 | 14394 | 6488 | −1782 | 1571 | | | 2760 | A | C |
| ATOM | 1394 | O | HIS A | 221 | 47.742 | 25.322 | 20.555 | | | 1.00 | 75.41 | | A | O |
| ANISOU | 1394 | O | HIS A | 221 | 6752 | 14848 | 7051 | −1783 | 1767 | | | 2783 | A | O |
| ATOM | 1395 | CB | HIS A | 221 | 45.612 | 25.682 | 18.950 | | | 1.00 | 80.28 | | A | C |
| ANISOU | 1395 | CB | HIS A | 221 | 7463 | 16072 | 6966 | −1727 | 1628 | | | 2834 | A | C |
| ATOM | 1396 | CG | HIS A | 221 | 46.214 | 24.377 | 18.536 | | | 1.00 | 79.83 | | A | C |
| ANISOU | 1396 | CG | HIS A | 221 | 7346 | 16262 | 6722 | −1778 | 1641 | | | 2483 | A | C |
| ATOM | 1397 | ND1 | HIS A | 221 | 45.895 | 23.190 | 19.160 | | | 1.00 | 75.02 | | A | N |
| ANISOU | 1397 | ND1 | HIS A | 221 | 6767 | 15744 | 5991 | −1813 | 1476 | | | 2269 | A | N |
| ATOM | 1398 | CD2 | HIS A | 221 | 47.124 | 24.068 | 17.584 | | | 1.00 | 81.45 | | A | C |
| ANISOU | 1398 | CD2 | HIS A | 221 | 7467 | 16625 | 6855 | −1801 | 1805 | | | 2285 | A | C |
| ATOM | 1399 | CE1 | HIS A | 221 | 46.576 | 22.205 | 18.605 | | | 1.00 | 75.67 | | A | C |
| ANISOU | 1399 | CE1 | HIS A | 221 | 6795 | 16027 | 5930 | −1850 | 1541 | | | 1949 | A | C |
| ATOM | 1400 | NE2 | HIS A | 221 | 47.328 | 22.711 | 17.645 | | | 1.00 | 78.84 | | A | N |
| ANISOU | 1400 | NE2 | HIS A | 221 | 7120 | 16475 | 6359 | −1838 | 1732 | | | 1952 | A | N |
| ATOM | 1401 | N | ASN A | 222 | 46.660 | 25.377 | 22.493 | | | 1.00 | 68.73 | | A | N |
| ANISOU | 1401 | N | ASN A | 222 | 5966 | 13853 | 6295 | −1816 | 1452 | | | 2604 | A | N |
| ATOM | 1402 | CA | ASN A | 222 | 47.888 | 25.104 | 23.217 | | | 1.00 | 80.29 | | A | C |
| ANISOU | 1402 | CA | ASN A | 222 | 7324 | 15174 | 8009 | −1842 | 1543 | | | 2393 | A | C |
| ATOM | 1403 | C | ASN A | 222 | 48.243 | 23.616 | 23.233 | | | 1.00 | 92.14 | | A | C |
| ANISOU | 1403 | C | ASN A | 222 | 8757 | 16963 | 9290 | −1876 | 1597 | | | 2052 | A | C |
| ATOM | 1404 | O | ASN A | 222 | 49.369 | 23.240 | 23.556 | | | 1.00 | 103.64 | | A | O |
| ANISOU | 1404 | O | ASN A | 222 | 10096 | 18380 | 10903 | −1879 | 1710 | | | 1843 | A | O |
| ATOM | 1405 | CE | ASN A | 222 | 47.808 | 25.654 | 24.643 | | | 1.00 | 98.25 | | A | C |
| ANISOU | 1405 | CB | ASN A | 222 | 9618 | 17095 | 10615 | −1840 | 1396 | | | 2385 | A | C |
| ATOM | 1406 | CG | ASN A | 222 | 49.181 | 25.937 | 25.238 | | | 1.00 | 113.99 | | A | C |
| ANISOU | 1406 | CG | ASN A | 222 | 11580 | 18733 | 12999 | −1805 | 1457 | | | 2539 | A | C |
| ATOM | 1407 | OD1 | ASN A | 222 | 50.210 | 25.565 | 24.668 | | | 1.00 | 116.43 | | A | O |
| ANISOU | 1407 | OD1 | ASN A | 222 | 11767 | 18922 | 13551 | −1804 | 1563 | | | 2384 | A | O |
| ATOM | 1408 | ND2 | ASN A | 222 | 49.202 | 26.601 | 26.386 | | | 1.00 | 116.50 | | A | N |
| ANISOU | 1408 | ND2 | ASN A | 222 | 11998 | 18890 | 13375 | −1770 | 1391 | | | 2809 | A | N |
| ATOM | 1409 | N | PHE A | 223 | 47.294 | 22.782 | 22.834 | | | 1.00 | 87.72 | | A | N |
| ANISOU | 1409 | N | PHE A | 223 | 8260 | 16704 | 8366 | −1890 | 1531 | | | 1971 | A | N |
| ATOM | 1410 | CA | PHE A | 223 | 47.448 | 21.321 | 22.858 | | | 1.00 | 82.07 | | A | C |
| ANISOU | 1410 | CA | PHE A | 223 | 7553 | 16124 | 7506 | −1915 | 1483 | | | 1602 | A | C |
| ATOM | 1411 | C | PHE A | 223 | 48.659 | 20.641 | 22.211 | | | 1.00 | 77.37 | | A | C |
| ANISOU | 1411 | C | PHE A | 223 | 6875 | 15736 | 6785 | −1904 | 1630 | | | 1312 | A | C |
| ATOM | 1412 | O | PHE A | 223 | 49.150 | 19.653 | 22.747 | | | 1.00 | 70.48 | | A | O |
| ANISOU | 1412 | O | PHE A | 223 | 6062 | 14768 | 5949 | −1865 | 1553 | | | 939 | A | O |
| ATOM | 1413 | CB | PHE A | 223 | 46.174 | 20.672 | 22.311 | | | 1.00 | 81.91 | | A | C |
| ANISOU | 1413 | CB | PHE A | 223 | 7637 | 16310 | 7175 | −1940 | 1343 | | | 1589 | A | C |
| ATOM | 1414 | CG | PHE A | 223 | 44.924 | 21.132 | 22.997 | | | 1.00 | 78.81 | | A | C |
| ANISOU | 1414 | CG | PHE A | 223 | 7330 | 15706 | 6908 | −1949 | 1178 | | | 1819 | A | C |
| ATOM | 1415 | CD1 | PHE A | 223 | 44.352 | 20.377 | 24.002 | | | 1.00 | 73.06 | | A | C |
| ANISOU | 1415 | CD1 | PHE A | 223 | 6697 | 14567 | 6494 | −1924 | 1023 | | | 1682 | A | C |
| ATOM | 1416 | CD2 | PHE A | 223 | 44.325 | 22.326 | 22.640 | | | 1.00 | 83.58 | | A | C |
| ANISOU | 1416 | CD2 | PHE A | 223 | 7985 | 16361 | 7411 | −1906 | 1103 | | | 2108 | A | C |
| ATOM | 1417 | CE1 | PHE A | 223 | 43.202 | 20.802 | 24.635 | | | 1.00 | 81.86 | | A | C |
| ANISOU | 1417 | CE1 | PHE A | 223 | 7887 | 15490 | 7726 | −1936 | 873 | | | 1888 | A | C |
| ATOM | 1418 | CE2 | PHE A | 223 | 43.176 | 22.759 | 23.268 | | | 1.00 | 71.01 | | A | C |
| ANISOU | 1418 | CE2 | PHE A | 223 | 6472 | 14552 | 5957 | −1899 | 938 | | | 2293 | A | C |
| ATOM | 1419 | CZ | PHE A | 223 | 42.613 | 21.996 | 24.267 | | | 1.00 | 74.79 | | A | C |
| ANISOU | 1419 | CZ | PHE A | 223 | 6988 | 14762 | 6668 | −1944 | 850 | | | 2210 | A | C |
| ATOM | 1420 | N | THR A | 224 | 49.140 | 21.116 | 21.073 | | | 1.00 | 85.28 | | A | N |
| ANISOU | 1420 | N | THR A | 224 | 7792 | 16921 | 7691 | −1902 | 1806 | | | 1438 | A | N |
| ATOM | 1421 | CA | THR A | 224 | 50.284 | 20.441 | 20.460 | | | 1.00 | 91.47 | | A | C |
| ANISOU | 1421 | CA | THR A | 224 | 8474 | 17882 | 8399 | −1900 | 1955 | | | 1139 | A | C |
| ATOM | 1422 | C | THR A | 224 | 51.539 | 20.448 | 21.337 | | | 1.00 | 93.58 | | A | C |
| ANISOU | 1422 | C | THR A | 224 | 8645 | 17868 | 9045 | −1871 | 2016 | | | 989 | A | C |
| ATOM | 1423 | O | THR A | 224 | 52.255 | 19.452 | 21.403 | | | 1.00 | 95.06 | | A | O |
| ANISOU | 1423 | O | THR A | 224 | 8798 | 18057 | 9262 | −1825 | 2043 | | | 635 | A | O |
| ATOM | 1424 | CB | THR A | 224 | 50.640 | 21.024 | 19.079 | | | 1.00 | 84.32 | | A | C |
| ANISOU | 1424 | CB | THR A | 224 | 7541 | 17200 | 7298 | −1886 | 2100 | | | 1264 | A | C |
| ATOM | 1425 | OG1 | THR A | 224 | 50.570 | 22.454 | 19.127 | | | 1.00 | 80.96 | | A | O |
| ANISOU | 1425 | OG1 | THR A | 224 | 7151 | 16617 | 6993 | −1870 | 2137 | | | 1657 | A | O |
| ATOM | 1426 | CG2 | THR A | 224 | 49.686 | 20.506 | 18.021 | | | 1.00 | 80.74 | | A | C |
| ANISOU | 1426 | CG2 | THR A | 224 | 7144 | 17094 | 6438 | −1882 | 2037 | | | 1163 | A | C |
| ATOM | 1427 | N | ALA A | 225 | 51.810 | 21.568 | 21.997 | | | 1.00 | 90.56 | | A | N |
| ANISOU | 1427 | N | ALA A | 225 | 8231 | 17206 | 8972 | −1874 | 2013 | | | 1226 | A | N |
| ATOM | 1428 | CA | ALA A | 225 | 52.997 | 21.684 | 22.844 | | | 1.00 | 92.73 | | A | C |
| ANISOU | 1428 | CA | ALA A | 225 | 8390 | 17230 | 9611 | −1835 | 2084 | | | 1092 | A | C |
| ATOM | 1429 | C | ALA A | 225 | 52.780 | 21.585 | 24.358 | | | 1.00 | 83.47 | | A | C |
| ANISOU | 1429 | C | ALA A | 225 | 7293 | 15633 | 8788 | −1759 | 1881 | | | 1006 | A | C |
| ATOM | 1430 | O | ALA A | 225 | 53.715 | 21.804 | 25.124 | | | 1.00 | 90.51 | | A | O |
| ANISOU | 1430 | O | ALA A | 225 | 8082 | 16302 | 10006 | −1713 | 1917 | | | 913 | A | O |
| ATOM | 1431 | CB | ALA A | 225 | 53.750 | 22.964 | 22.510 | | | 1.00 | 96.06 | | A | C |
| ANISOU | 1431 | CB | ALA A | 225 | 8739 | 17582 | 10176 | −1865 | 2262 | | | 1372 | A | C |
| ATOM | 1432 | N | SER A | 226 | 51.568 | 21.269 | 24.798 | | | 1.00 | 72.08 | | A | N |
| ANISOU | 1432 | N | SER A | 226 | 6022 | 14081 | 7286 | −1742 | 1673 | | | 1015 | A | N |
| ATOM | 1433 | CA | SER A | 226 | 51.296 | 21.208 | 26.238 | | | 1.00 | 64.73 | | A | C |
| ANISOU | 1433 | CA | SER A | 226 | 5168 | 12748 | 6676 | −1677 | 1493 | | | 1008 | A | C |
| ATOM | 1434 | C | SER A | 226 | 51.233 | 19.846 | 26.918 | | | 1.00 | 62.92 | | A | C |
| ANISOU | 1434 | C | SER A | 226 | 5097 | 12346 | 6463 | −1579 | 1318 | | | 652 | A | C |
| ATOM | 1435 | O | SER A | 226 | 50.841 | 19.770 | 28.076 | | | 1.00 | 60.54 | | A | O |
| ANISOU | 1435 | O | SER A | 226 | 4930 | 11778 | 6295 | −1543 | 1141 | | | 672 | A | O |
| ATOM | 1436 | CB | SER A | 226 | 50.026 | 21.985 | 26.585 | | | 1.00 | 63.08 | | A | C |
| ANISOU | 1436 | CB | SER A | 226 | 5029 | 12475 | 6463 | −1740 | 1404 | | | 1378 | A | C |
| ATOM | 1437 | OG | SER A | 226 | 48.901 | 21.412 | 25.951 | | | 1.00 | 62.59 | | A | O |
| ANISOU | 1437 | OG | SER A | 226 | 5122 | 12523 | 6137 | −1760 | 1268 | | | 1341 | A | O |
| ATOM | 1438 | N | THR A | 227 | 51.600 | 18.767 | 26.244 | | | 1.00 | 64.35 | | A | N |
| ANISOU | 1438 | N | THR A | 227 | 5271 | 12659 | 6519 | −1531 | 1374 | | | 325 | A | N |
| ATOM | 1439 | CA | THR A | 227 | 51.485 | 17.485 | 26.924 | | | 1.00 | 63.11 | | A | C |
| ANISOU | 1439 | CA | THR A | 227 | 5277 | 12299 | 6403 | −1422 | 1231 | | | −14 | A | C |
| ATOM | 1440 | C | THR A | 227 | 52.078 | 17.577 | 28.331 | | | 1.00 | 61.15 | | A | C |
| ANISOU | 1440 | C | THR A | 227 | 5040 | 11662 | 6533 | −1284 | 1136 | | | −110 | A | C |
| ATOM | 1441 | O | THR A | 227 | 53.120 | 18.202 | 28.533 | | | 1.00 | 61.68 | | A | O |
| ANISOU | 1441 | O | THR A | 227 | 4937 | 11695 | 6804 | −1247 | 1226 | | | −102 | A | O |
| ATOM | 1442 | CB | THR A | 227 | 52.178 | 16.413 | 26.097 | | | 1.00 | 65.51 | | A | C |
| ANISOU | 1442 | CB | THR A | 227 | 5557 | 12824 | 6511 | −1391 | 1329 | | | −344 | A | C |
| ATOM | 1443 | OG1 | THR A | 227 | 51.631 | 16.424 | 24.760 | | | 1.00 | 67.26 | | A | O |
| ANISOU | 1443 | OG1 | THR A | 227 | 5748 | 13439 | 6370 | −1517 | 1421 | | | −242 | A | O |
| ATOM | 1444 | CG2 | THR A | 227 | 52.012 | 15.042 | 26.760 | | | 1.00 | 64.24 | | A | C |
| ANISOU | 1444 | CG2 | THR A | 227 | 5588 | 12439 | 6380 | −1274 | 1202 | | | −691 | A | C |
| ATOM | 1445 | N | ASN A | 228 | 51.384 | 16.990 | 29.316 | | | 1.00 | 60.48 | | A | N |
| ANISOU | 1445 | N | ASN A | 228 | 5151 | 11289 | 6541 | −1207 | 959 | | | −199 | A | N |
| ATOM | 1446 | CA | ASN A | 228 | 51.729 | 17.108 | 30.743 | | | 1.00 | 56.94 | | A | C |
| ANISOU | 1446 | CA | ASN A | 228 | 4742 | 10463 | 6430 | −1061 | 841 | | | −257 | A | C |
| ATOM | 1447 | C | ASN A | 228 | 51.839 | 18.567 | 31.179 | | | 1.00 | 56.10 | | A | C |
| ANISOU | 1447 | C | ASN A | 228 | 4483 | 10276 | 6555 | −1110 | 855 | | | 53 | A | C |
| ATOM | 1448 | O | ASN A | 228 | 52.673 | 18.921 | 32.013 | | | 1.00 | 55.52 | | A | O |
| ANISOU | 1448 | O | ASN A | 228 | 4315 | 10015 | 6765 | −1001 | 840 | | | −16 | A | O |
| ATOM | 1449 | CB | ASN A | 228 | 53.012 | 16.329 | 31.087 | | | 1.00 | 57.75 | | A | C |
| ANISOU | 1449 | CB | ASN A | 228 | 4808 | 10483 | 6650 | −880 | 867 | | | −615 | A | C |
| ATOM | 1450 | CG | ASN A | 228 | 52.849 | 14.815 | 30.882 | | | 1.00 | 71.18 | | A | C |
| ANISOU | 1450 | CG | ASN A | 228 | 6699 | 12180 | 8166 | −804 | 838 | | | −934 | A | C |
| ATOM | 1451 | OD1 | ASN A | 228 | 51.733 | 14.296 | 30.974 | | | 1.00 | 72.60 | | A | O |
| ANISOU | 1451 | OD1 | ASN A | 228 | 7076 | 12284 | 8226 | −852 | 755 | | | −917 | A | O |
| ATOM | 1452 | ND2 | ASN A | 228 | 53.969 | 14.104 | 30.608 | | | 1.00 | 99.89 | | A | N |
| ANISOU | 1452 | ND2 | ASN A | 228 | 10272 | 15894 | 11787 | −689 | 917 | | | −1238 | A | N |
| ATOM | 1453 | N | ARG A | 229 | 51.023 | 19.424 | 30.571 | | | 1.00 | 56.26 | | A | N |
| ANISOU | 1453 | N | ARG A | 229 | 4472 | 10455 | 6449 | −1269 | 894 | | | 384 | A | N |
| ATOM | 1454 | CA | ARG A | 229 | 50.807 | 20.794 | 30.998 | | | 1.00 | 55.28 | | A | C |
| ANISOU | 1454 | CA | ARG A | 229 | 4252 | 10227 | 6525 | −1330 | 895 | | | 716 | A | C |
| ATOM | 1455 | C | ARG A | 229 | 49.310 | 21.022 | 31.159 | | | 1.00 | 53.69 | | A | C |
| ANISOU | 1455 | C | ARG A | 229 | 4202 | 9976 | 6224 | −1409 | 763 | | | 952 | A | C |
| ATOM | 1456 | O | ARG A | 229 | 48.476 | 20.320 | 30.583 | | | 1.00 | 54.03 | | A | O |
| ANISOU | 1456 | O | ARG A | 229 | 4371 | 10168 | 5991 | −1458 | 723 | | | 903 | A | O |
| ATOM | 1457 | CB | ARG A | 229 | 51.378 | 21.812 | 30.015 | | | 1.00 | 57.50 | | A | C |
| ANISOU | 1457 | CB | ARG A | 229 | 4327 | 10763 | 6757 | −1442 | 1111 | | | 931 | A | C |
| ATOM | 1458 | CG | ARG A | 229 | 52.868 | 21.938 | 30.115 | | | 1.00 | 62.06 | | A | C |
| ANISOU | 1458 | CG | ARG A | 229 | 4715 | 11328 | 7535 | −1379 | 1243 | | | 741 | A | C |
| ATOM | 1459 | CD | ARG A | 229 | 53.415 | 23.161 | 29.386 | | | 1.00 | 63.73 | | A | C |
| ANISOU | 1459 | CD | ARG A | 229 | 4721 | 11708 | 7784 | −1500 | 1474 | | | 990 | A | C |
| ATOM | 1460 | NE | ARG A | 229 | 54.770 | 23.465 | 29.843 | | | 1.00 | 61.42 | | A | N |
| ANISOU | 1460 | NE | ARG A | 229 | 4232 | 11318 | 7787 | −1441 | 1575 | | | 809 | A | N |
| ATOM | 1461 | CZ | ARG A | 229 | 55.861 | 22.800 | 29.463 | | | 1.00 | 75.74 | | A | C |
| ANISOU | 1461 | CZ | ARG A | 229 | 5947 | 13261 | 9569 | −1381 | 1672 | | | 496 | A | C |
| ATOM | 1462 | NH1 | ARG A | 229 | 55.763 | 21.795 | 28.599 | | | 1.00 | 75.41 | | A | N1+ |
| ANISOU | 1462 | NH1 | ARG A | 229 | 5992 | 13448 | 9212 | −1379 | 1688 | | | 341 | A | N1+ |
| ATOM | 1463 | NH2 | ARG A | 229 | 57.051 | 23.146 | 29.946 | | | 1.00 | 78.66 | | A | N |
| ANISOU | 1463 | NH2 | ARG A | 229 | 6120 | 13542 | 10225 | −1325 | 1754 | | | 323 | A | N |
| ATOM | 1464 | N | SER A | 230 | 48.955 | 21.974 | 32.006 | | | 1.00 | 56.81 | | A | N |
| ANISOU | 1464 | N | SER A | 230 | 4568 | 10167 | 6850 | −1423 | 698 | | | 1194 | A | N |
| ATOM | 1465 | CA | SER A | 230 | 47.556 | 22.214 | 32.290 | | | 1.00 | 50.24 | | A | C |
| ANISOU | 1465 | CA | SER A | 230 | 3879 | 9234 | 5978 | −1475 | 548 | | | 1387 | A | C |
| ATOM | 1466 | C | SER A | 230 | 46.873 | 23.208 | 31.375 | | | 1.00 | 51.39 | | A | C |
| ANISOU | 1466 | C | SER A | 230 | 3952 | 9627 | 5945 | −1612 | 635 | | | 1751 | A | C |
| ATOM | 1467 | O | SER A | 230 | 47.416 | 24.257 | 31.060 | | | 1.00 | 52.25 | | A | O |
| ANISOU | 1467 | O | SER A | 230 | 3905 | 9769 | 6178 | −1653 | 765 | | | 1974 | A | O |
| ATOM | 1468 | CB | SER A | 230 | 47.382 | 22.631 | 33.743 | | | 1.00 | 47.73 | | A | C |
| ANISOU | 1468 | CB | SER A | 230 | 3587 | 8542 | 6005 | −1396 | 409 | | | 1430 | A | C |
| ATOM | 1469 | OG | SER A | 230 | 47.887 | 23.922 | 33.939 | | | 1.00 | 54.39 | | A | O |
| ANISOU | 1469 | OG | SER A | 230 | 4278 | 9370 | 7019 | −1464 | 478 | | | 1739 | A | O |
| ATOM | 1470 | N | VAL A | 231 | 45.649 | 22.857 | 30.995 | | | 1.00 | 51.59 | | A | N |
| ANISOU | 1470 | N | VAL A | 231 | 4095 | 9830 | 5677 | −1676 | 572 | | | 1795 | A | N |
| ATOM | 1471 | CA | VAL A | 231 | 44.806 | 23.647 | 30.113 | | | 1.00 | 52.52 | | A | C |
| ANISOU | 1471 | CA | VAL A | 231 | 4191 | 10190 | 5574 | −1775 | 603 | | | 2122 | A | C |
| ATOM | 1472 | C | VAL A | 231 | 43.419 | 23.784 | 30.724 | | | 1.00 | 50.28 | | A | C |
| ANISOU | 1472 | C | VAL A | 231 | 4034 | 9736 | 5335 | −1795 | 411 | | | 2251 | A | C |
| ATOM | 1473 | O | VAL A | 231 | 43.089 | 23.094 | 31.679 | | | 1.00 | 48.05 | | A | O |
| ANISOU | 1473 | O | VAL A | 231 | 3850 | 9137 | 5268 | −1739 | 272 | | | 2104 | A | O |
| ATOM | 1474 | CB | VAL A | 231 | 44.691 | 23.017 | 28.719 | | | 1.00 | 54.92 | | A | C |
| ANISOU | 1474 | CB | VAL A | 231 | 4505 | 10895 | 5469 | −1819 | 683 | | | 2023 | A | C |
| ATOM | 1475 | CG1 | VAL A | 231 | 45.988 | 23.187 | 27.953 | | | 1.00 | 57.44 | | A | C |
| ANISOU | 1475 | CG1 | VAL A | 231 | 4676 | 11428 | 5722 | −1819 | 900 | | | 1989 | A | C |
| ATOM | 1476 | CG2 | VAL A | 231 | 44.319 | 21.552 | 28.829 | | | 1.00 | 54.30 | | A | C |
| ANISOU | 1476 | CG2 | VAL A | 231 | 4574 | 10777 | 5281 | −1791 | 572 | | | 1652 | A | C |
| ATOM | 1477 | N | LEU A | 232 | 42.635 | 24.719 | 30.207 | | | 1.00 | 51.03 | | A | N |
| ANISOU | 1477 | N | LEU A | 232 | 4127 | 10051 | 5209 | −1865 | 404 | | | 2521 | A | N |
| ATOM | 1478 | CA | LEU A | 232 | 41.290 | 24.966 | 30.700 | | | 1.00 | 49.17 | | A | C |
| ANISOU | 1478 | CA | LEU A | 232 | 4017 | 9634 | 5033 | −1849 | 213 | | | 2649 | A | C |
| ATOM | 1479 | C | LEU A | 232 | 40.163 | 24.390 | 29.846 | | | 1.00 | 50.12 | | A | C |
| ANISOU | 1479 | C | LEU A | 232 | 4196 | 10067 | 4782 | −1913 | 152 | | | 2640 | A | C |
| ATOM | 1480 | O | LEU A | 232 | 40.169 | 24.506 | 28.632 | | | 1.00 | 63.63 | | A | O |
| ANISOU | 1480 | O | LEU A | 232 | 5866 | 12081 | 6229 | −1899 | 227 | | | 2706 | A | O |
| ATOM | 1481 | CB | LEU A | 232 | 41.078 | 26.464 | 30.816 | | | 1.00 | 49.13 | | A | C |
| ANISOU | 1481 | CB | LEU A | 232 | 4026 | 9422 | 5219 | −1743 | 186 | | | 2886 | A | C |
| ATOM | 1482 | CG | LEU A | 232 | 42.043 | 27.197 | 31.732 | | | 1.00 | 47.52 | | A | C |
| ANISOU | 1482 | CG | LEU A | 232 | 3810 | 8827 | 5417 | −1664 | 180 | | | 2887 | A | C |
| ATOM | 1483 | CD1 | LEU A | 232 | 42.076 | 28.667 | 31.371 | | | 1.00 | 51.45 | | A | C |
| ANISOU | 1483 | CD1 | LEU A | 232 | 4299 | 9225 | 6023 | −1582 | 228 | | | 3085 | A | C |
| ATOM | 1484 | CD2 | LEU A | 232 | 41.611 | 27.012 | 33.172 | | | 1.00 | 44.54 | | A | C |
| ANISOU | 1484 | CD2 | LEU A | 232 | 3541 | 8151 | 5232 | −1635 | −13 | | | 2826 | A | C |
| ATOM | 1485 | N | ILE A | 233 | 39.205 | 23.754 | 30.513 | | | 1.00 | 48.36 | | A | N |
| ANISOU | 1485 | N | ILE A | 233 | 4081 | 9738 | 4555 | −1957 | 3 | | | 2525 | A | N |
| ATOM | 1486 | CA | ILE A | 233 | 38.008 | 23.176 | 29.913 | | | 1.00 | 49.07 | | A | C |
| ANISOU | 1486 | CA | ILE A | 233 | 4238 | 10078 | 4328 | −2013 | −77 | | | 2431 | A | C |
| ATOM | 1487 | C | ILE A | 233 | 36.832 | 24.078 | 30.282 | | | 1.00 | 51.00 | | A | C |
| ANISOU | 1487 | C | ILE A | 233 | 4519 | 10221 | 4638 | −1994 | −235 | | | 2650 | A | C |
| ATOM | 1488 | O | ILE A | 233 | 36.416 | 24.092 | 31.453 | | | 1.00 | 45.43 | | A | O |
| ANISOU | 1488 | O | ILE A | 233 | 3896 | 9193 | 4172 | −1998 | −356 | | | 2637 | A | O |
| ATOM | 1489 | CB | ILE A | 233 | 37.753 | 21.740 | 30.396 | | | 1.00 | 48.14 | | A | C |
| ANISOU | 1489 | CB | ILE A | 233 | 4265 | 9810 | 4216 | −2023 | −153 | | | 2024 | A | C |
| ATOM | 1490 | CG1 | ILE A | 233 | 38.602 | 20.702 | 29.690 | | | 1.00 | 49.95 | | A | C |
| ANISOU | 1490 | CG1 | ILE A | 233 | 4485 | 10197 | 4298 | −2006 | −32 | | | 1723 | A | C |
| ATOM | 1491 | CG2 | ILE A | 233 | 36.364 | 21.331 | 30.050 | | | 1.00 | 48.44 | | A | C |
| ANISOU | 1491 | CG2 | ILE A | 233 | 4363 | 10034 | 4007 | −2096 | −253 | | | 1945 | A | C |
| ATOM | 1492 | CD1 | ILE A | 233 | 40.039 | 21.006 | 29.647 | | | 1.00 | 50.56 | | A | C |
| ANISOU | 1492 | CD1 | ILE A | 233 | 4464 | 10219 | 4526 | −1940 | 100 | | | 1756 | A | C |
| ATOM | 1493 | N | PRO A | 234 | 36.271 | 24.863 | 29.341 | | | 1.00 | 50.40 | | A | N |
| ANISOU | 1493 | N | PRO A | 234 | 4388 | 10352 | 4409 | −1931 | −256 | | | 2796 | A | N |
| ATOM | 1494 | CA | PRO A | 234 | 35.116 | 25.691 | 29.696 | | | 1.00 | 49.30 | | A | C |
| ANISOU | 1494 | CA | PRO A | 234 | 4264 | 10082 | 4387 | −1881 | −412 | | | 2915 | A | C |
| ATOM | 1495 | C | PRO A | 234 | 33.828 | 24.891 | 29.617 | | | 1.00 | 51.11 | | A | C |
| ANISOU | 1495 | C | PRO A | 234 | 4534 | 10477 | 4408 | −1975 | −514 | | | 2755 | A | C |
| ATOM | 1496 | O | PRO A | 234 | 33.408 | 24.463 | 28.530 | | | 1.00 | 51.09 | | A | O |
| ANISOU | 1496 | O | PRO A | 234 | 4486 | 10838 | 4086 | −1999 | −477 | | | 2666 | A | O |
| ATOM | 1497 | CB | PRO A | 234 | 35.144 | 26.813 | 28.649 | | | 1.00 | 51.01 | | A | C |
| ANISOU | 1497 | CB | PRO A | 234 | 4388 | 10460 | 4532 | −1776 | −347 | | | 3114 | A | C |
| ATOM | 1498 | CG | PRO A | 234 | 36.390 | 26.584 | 27.853 | | | 1.00 | 53.06 | | A | C |
| ANISOU | 1498 | CG | PRO A | 234 | 4603 | 10894 | 4664 | −1772 | −165 | | | 3110 | A | C |
| ATOM | 1499 | CD | PRO A | 234 | 36.704 | 25.143 | 27.970 | | | 1.00 | 52.71 | | A | C |
| ANISOU | 1499 | CD | PRO A | 234 | 4590 | 10969 | 4467 | −1886 | −126 | | | 2860 | A | C |
| ATOM | 1500 | N | VAL A | 235 | 33.207 | 24.699 | 30.783 | | | 1.00 | 50.00 | | A | N |
| ANISOU | 1500 | N | VAL A | 235 | 4482 | 10053 | 4461 | −2019 | −639 | | | 2696 | A | N |
| ATOM | 1501 | CA | VAL A | 235 | 32.033 | 23.853 | 30.916 | | | 1.00 | 52.81 | | A | C |
| ANISOU | 1501 | CA | VAL A | 235 | 4905 | 10495 | 4664 | −2128 | −721 | | | 2506 | A | C |
| ATOM | 1502 | C | VAL A | 235 | 30.898 | 24.636 | 31.573 | | | 1.00 | 52.27 | | A | C |
| ANISOU | 1502 | C | VAL A | 235 | 4832 | 10207 | 4823 | −2103 | −835 | | | 2581 | A | C |
| ATOM | 1503 | O | VAL A | 235 | 31.109 | 25.398 | 32.533 | | | 1.00 | 62.35 | | A | O |
| ANISOU | 1503 | O | VAL A | 235 | 6117 | 11143 | 6431 | −2034 | −897 | | | 2689 | A | O |
| ATOM | 1504 | CB | VAL A | 235 | 32.366 | 22.559 | 31.692 | | | 1.00 | 49.50 | | A | C |
| ANISOU | 1504 | CB | VAL A | 235 | 4641 | 9944 | 4224 | −2236 | −720 | | | 2285 | A | C |
| ATOM | 1505 | CG1 | VAL A | 235 | 32.413 | 22.794 | 33.193 | | | 1.00 | 41.14 | | A | C |
| ANISOU | 1505 | CG1 | VAL A | 235 | 3676 | 8401 | 3555 | −2216 | −807 | | | 2332 | A | C |
| ATOM | 1506 | CG2 | VAL A | 235 | 31.382 | 21.471 | 31.327 | | | 1.00 | 62.52 | | A | C |
| ANISOU | 1506 | CG2 | VAL A | 235 | 6351 | 11764 | 5639 | −2320 | −765 | | | 1954 | A | C |
| ATOM | 1507 | N | THR A | 236 | 29.695 | 24.471 | 31.008 | | | 1.00 | 53.61 | | A | N |
| ANISOU | 1507 | N | THR A | 236 | 4981 | 10579 | 4808 | −2141 | −845 | | | 2491 | A | N |
| ATOM | 1508 | CA | THR A | 236 | 28.501 | 25.154 | 31.505 | | | 1.00 | 65.00 | | A | C |
| ANISOU | 1508 | CA | THR A | 236 | 6426 | 11851 | 6420 | −2127 | −901 | | | 2538 | A | C |
| ATOM | 1509 | C | THR A | 236 | 28.045 | 24.560 | 32.827 | | | 1.00 | 75.05 | | A | C |
| ANISOU | 1509 | C | THR A | 236 | 7859 | 12779 | 7876 | −2215 | −955 | | | 2410 | A | C |
| ATOM | 1510 | O | THR A | 236 | 27.655 | 25.286 | 33.753 | | | 1.00 | 79.19 | | A | O |
| ANISOU | 1510 | O | THR A | 236 | 8419 | 12979 | 8690 | −2173 | −992 | | | 2482 | A | O |
| ATOM | 1511 | CB | THR A | 236 | 27.379 | 25.032 | 30.477 | | | 1.00 | 60.90 | | A | C |
| ANISOU | 1511 | CB | THR A | 236 | 5859 | 11662 | 5619 | −2122 | −881 | | | 2452 | A | C |
| ATOM | 1512 | OG1 | THR A | 236 | 27.306 | 23.658 | 30.069 | | | 1.00 | 67.49 | | A | O |
| ANISOU | 1512 | OG1 | THR A | 236 | 6751 | 12709 | 6182 | −2214 | −864 | | | 2174 | A | O |
| ATOM | 1513 | CG2 | THR A | 236 | 27.648 | 25.925 | 29.273 | | | 1.00 | 55.56 | | A | C |
| ANISOU | 1513 | CG2 | THR A | 236 | 5041 | 11260 | 4809 | −1999 | −830 | | | 2635 | A | C |
| ATOM | 1514 | N | SER A | 237 | 28.046 | 23.236 | 32.914 | | | 1.00 | 70.58 | | A | N |
| ANISOU | 1514 | N | SER A | 237 | 7423 | 12258 | 7135 | −2305 | −955 | | | 2172 | A | N |
| ATOM | 1515 | CA | SER A | 237 | 27.749 | 22.543 | 34.157 | | | 1.00 | 68.98 | | A | C |
| ANISOU | 1515 | CA | SER A | 237 | 7428 | 11694 | 7086 | −2351 | −1004 | | | 2024 | A | C |
| ATOM | 1516 | C | SER A | 237 | 28.769 | 21.429 | 34.394 | | | 1.00 | 60.64 | | A | C |
| ANISOU | 1516 | C | SER A | 237 | 6451 | 10597 | 5992 | −2394 | −1025 | | | 1828 | A | C |
| ATOM | 1517 | O | SER A | 237 | 29.389 | 20.915 | 33.462 | | | 1.00 | 56.52 | | A | O |
| ANISOU | 1517 | O | SER A | 237 | 5844 | 10400 | 5231 | −2429 | −989 | | | 1726 | A | O |
| ATOM | 1518 | CB | SER A | 237 | 26.331 | 21.968 | 34.133 | | | 1.00 | 65.84 | | A | C |
| ANISOU | 1518 | CB | SER A | 237 | 7116 | 11322 | 6579 | −2394 | −1016 | | | 1791 | A | C |
| ATOM | 1519 | OG | SER A | 237 | 26.175 | 21.121 | 33.011 | | | 1.00 | 66.90 | | A | O |
| ANISOU | 1519 | OG | SER A | 237 | 7179 | 11836 | 6404 | −2452 | −983 | | | 1552 | A | O |
| ATOM | 1520 | N | LEU A | 238 | 28.921 | 21.050 | 35.659 | | | 1.00 | 54.51 | | A | N |
| ANISOU | 1520 | N | LEU A | 238 | 5837 | 9400 | 5475 | −2375 | −1070 | | | 1741 | A | N |
| ATOM | 1521 | CA | LEU A | 238 | 29.810 | 19.976 | 36.056 | | | 1.00 | 47.92 | | A | C |
| ANISOU | 1521 | CA | LEU A | 238 | 5070 | 8408 | 4731 | −2406 | −1054 | | | 1495 | A | C |
| ATOM | 1522 | C | LEU A | 238 | 29.481 | 18.687 | 35.297 | | | 1.00 | 49.37 | | A | C |
| ANISOU | 1522 | C | LEU A | 238 | 5305 | 8785 | 4667 | −2489 | −972 | | | 1118 | A | C |
| ATOM | 1523 | O | LEU A | 238 | 28.369 | 18.154 | 35.444 | | | 1.00 | 53.99 | | A | O |
| ANISOU | 1523 | O | LEU A | 238 | 5959 | 9366 | 5188 | −2596 | −1012 | | | 924 | A | O |
| ATOM | 1524 | CB | LEU A | 238 | 29.710 | 19.762 | 37.563 | | | 1.00 | 44.25 | | A | C |
| ANISOU | 1524 | CB | LEU A | 238 | 4802 | 7429 | 4583 | −2342 | −1092 | | | 1438 | A | C |
| ATOM | 1525 | CG | LEU A | 238 | 30.839 | 18.912 | 38.175 | | | 1.00 | 59.77 | | A | C |
| ANISOU | 1525 | CG | LEU A | 238 | 6878 | 9110 | 6723 | −2275 | −1028 | | | 1251 | A | C |
| ATOM | 1526 | CD1 | LEU A | 238 | 32.191 | 19.599 | 38.034 | | | 1.00 | 61.15 | | A | C |
| ANISOU | 1526 | CD1 | LEU A | 238 | 6929 | 9304 | 6999 | −2152 | −983 | | | 1444 | A | C |
| ATOM | 1527 | CD2 | LEU A | 238 | 30.571 | 18.586 | 39.640 | | | 1.00 | 58.93 | | A | C |
| ANISOU | 1527 | CD2 | LEU A | 238 | 6966 | 8542 | 6884 | −2240 | −1085 | | | 1179 | A | C |
| ATOM | 1528 | N | PRO A | 239 | 30.414 | 18.148 | 34.510 | | | 1.00 | 48.71 | | A | N |
| ANISOU | 1528 | N | PRO A | 239 | 5186 | 8866 | 4456 | −2450 | −851 | | | 987 | A | N |
| ATOM | 1529 | CA | PRO A | 239 | 30.156 | 16.871 | 33.835 | | | 1.00 | 41.94 | | A | C |
| ANISOU | 1529 | CA | PRO A | 239 | 4380 | 8168 | 3387 | −2528 | −763 | | | 603 | A | C |
| ATOM | 1530 | C | PRO A | 239 | 29.867 | 15.740 | 34.807 | | | 1.00 | 41.46 | | A | C |
| ANISOU | 1530 | C | PRO A | 239 | 4545 | 7709 | 3499 | −2564 | −735 | | | 295 | A | C |
| ATOM | 1531 | O | PRO A | 239 | 30.485 | 15.632 | 35.868 | | | 1.00 | 39.14 | | A | O |
| ANISOU | 1531 | O | PRO A | 239 | 4386 | 7011 | 3474 | −2471 | −733 | | | 321 | A | O |
| ATOM | 1532 | CB | PRO A | 239 | 31.456 | 16.604 | 33.066 | | | 1.00 | 45.72 | | A | C |
| ANISOU | 1532 | CB | PRO A | 239 | 4792 | 8802 | 3777 | −2448 | −639 | | | 557 | A | C |
| ATOM | 1533 | CG | PRO A | 239 | 32.483 | 17.486 | 33.730 | | | 1.00 | 46.05 | | A | C |
| ANISOU | 1533 | CG | PRO A | 239 | 4808 | 8613 | 4075 | −2323 | −653 | | | 845 | A | C |
| ATOM | 1534 | CD | PRO A | 239 | 31.725 | 18.707 | 34.135 | | | 1.00 | 45.26 | | A | C |
| ANISOU | 1534 | CD | PRO A | 239 | 4643 | 8499 | 4053 | −2342 | −780 | | | 1174 | A | C |
| ATOM | 1535 | N | LYS A | 240 | 28.918 | 14.885 | 34.415 | | | 1.00 | 44.16 | | A | N |
| ANISOU | 1535 | N | LYS A | 240 | 4926 | 8178 | 3675 | −2695 | −703 | | | −7 | A | N |
| ATOM | 1536 | CA | LYS A | 240 | 28.680 | 13.597 | 35.068 | | | 1.00 | 48.55 | | A | C |
| ANISOU | 1536 | CA | LYS A | 240 | 5703 | 8401 | 4343 | −2747 | −617 | | | −362 | A | C |
| ATOM | 1537 | C | LYS A | 240 | 29.616 | 12.581 | 34.428 | | | 1.00 | 46.22 | | A | C |
| ANISOU | 1537 | C | LYS A | 240 | 5441 | 8167 | 3951 | −2708 | −466 | | | −625 | A | C |
| ATOM | 1538 | O | LYS A | 240 | 29.315 | 12.018 | 33.374 | | | 1.00 | 56.94 | | A | O |
| ANISOU | 1538 | O | LYS A | 240 | 6715 | 9863 | 5058 | −2799 | −399 | | | −856 | A | O |
| ATOM | 1539 | CB | LYS A | 240 | 27.218 | 13.168 | 34.939 | | | 1.00 | 53.94 | | A | C |
| ANISOU | 1539 | CB | LYS A | 240 | 6397 | 9188 | 4909 | −2924 | −633 | | | −588 | A | C |
| ATOM | 1540 | CG | LYS A | 240 | 26.191 | 14.176 | 35.460 | | | 1.00 | 52.00 | | A | C |
| ANISOU | 1540 | CG | LYS A | 240 | 6116 | 8871 | 4771 | −2881 | −738 | | | −320 | A | C |
| ATOM | 1541 | CD | LYS A | 240 | 26.283 | 14.383 | 36.947 | | | 1.00 | 54.48 | | A | C |
| ANISOU | 1541 | CD | LYS A | 240 | 6611 | 8681 | 5409 | −2804 | −781 | | | −188 | A | C |
| ATOM | 1542 | CE | LYS A | 240 | 25.614 | 15.680 | 37.378 | | | 1.00 | 54.97 | | A | C |
| ANISOU | 1542 | CE | LYS A | 240 | 6609 | 8707 | 5570 | −2699 | −885 | | | 154 | A | C |
| ATOM | 1543 | NZ | LYS A | 240 | 25.958 | 15.990 | 38.818 | | | 1.00 | 55.51 | | A | N1+ |
| ANISOU | 1543 | NZ | LYS A | 240 | 6832 | 8309 | 5950 | −2571 | −926 | | | 306 | A | N1+ |
| ATOM | 1544 | N | LEU A | 241 | 30.769 | 12.365 | 35.054 | | | 1.00 | 42.94 | | A | N |
| ANISOU | 1544 | N | LEU A | 241 | 5140 | 7445 | 3732 | −2561 | −418 | | | −598 | A | N |
| ATOM | 1545 | CA | LEU A | 241 | 31.767 | 11.481 | 34.478 | | | 1.00 | 44.65 | | A | C |
| ANISOU | 1545 | CA | LEU A | 241 | 5380 | 7714 | 3872 | −2500 | −283 | | | −825 | A | C |
| ATOM | 1546 | C | LEU A | 241 | 31.309 | 10.031 | 34.511 | | | 1.00 | 52.03 | | A | C |
| ANISOU | 1546 | C | LEU A | 241 | 6496 | 8496 | 4775 | −2587 | −155 | | | −1247 | A | C |
| ATOM | 1547 | O | LEU A | 241 | 30.438 | 9.643 | 35.304 | | | 1.00 | 49.75 | | A | O |
| ANISOU | 1547 | O | LEU A | 241 | 6372 | 7926 | 4604 | −2662 | −154 | | | −1356 | A | O |
| ATOM | 1548 | CB | LEU A | 241 | 33.075 | 11.602 | 35.233 | | | 1.00 | 43.41 | | A | C |
| ANISOU | 1548 | CB | LEU A | 241 | 5300 | 7252 | 3943 | −2306 | −272 | | | −710 | A | C |
| ATOM | 1549 | CG | LEU A | 241 | 33.764 | 12.954 | 35.233 | | | 1.00 | 45.29 | | A | C |
| ANISOU | 1549 | CG | LEU A | 241 | 5360 | 7598 | 4251 | −2211 | −360 | | | −330 | A | C |
| ATOM | 1550 | CD1 | LEU A | 241 | 35.139 | 12.734 | 35.839 | | | 1.00 | 41.88 | | A | C |
| ANISOU | 1550 | CD1 | LEU A | 241 | 4999 | 6897 | 4014 | −2022 | −316 | | | −348 | A | C |
| ATOM | 1551 | CD2 | LEU A | 241 | 33.847 | 13.528 | 33.814 | | | 1.00 | 50.48 | | A | C |
| ANISOU | 1551 | CD2 | LEU A | 241 | 5773 | 8776 | 4632 | −2271 | −343 | | | −220 | A | C |
| ATOM | 1552 | N | ASP A | 242 | 31.924 | 9.224 | 33.638 | | | 1.00 | 49.18 | | A | N |
| ANISOU | 1552 | N | ASP A | 242 | 6107 | 8317 | 4262 | −2580 | −31 | | | −1492 | A | N |
| ATOM | 1553 | CA | ASP A | 242 | 31.718 | 7.780 | 33.640 | | | 1.00 | 61.67 | | A | C |
| ANISOU | 1553 | CA | ASP A | 242 | 7869 | 9727 | 5835 | −2644 | 123 | | | −1909 | A | C |
| ATOM | 1554 | C | ASP A | 242 | 32.673 | 7.148 | 34.654 | | | 1.00 | 66.37 | | A | C |
| ANISOU | 1554 | C | ASP A | 242 | 8709 | 9836 | 6674 | −2467 | 195 | | | −1966 | A | C |
| ATOM | 1555 | O | ASP A | 242 | 33.891 | 7.191 | 34.471 | | | 1.00 | 74.39 | | A | O |
| ANISOU | 1555 | O | ASP A | 242 | 9683 | 10877 | 7705 | −2314 | 217 | | | −1913 | A | O |
| ATOM | 1556 | CB | ASP A | 242 | 31.924 | 7.194 | 32.241 | | | 1.00 | 68.49 | | A | C |
| ANISOU | 1556 | CB | ASP A | 242 | 8585 | 11013 | 6423 | −2712 | 223 | | | −2160 | A | C |
| ATOM | 1557 | CG | ASP A | 242 | 31.648 | 5.684 | 32.177 | | | 1.00 | 82.35 | | A | C |
| ANISOU | 1557 | CG | ASP A | 242 | 10516 | 12603 | 8172 | −2798 | 400 | | | −2619 | A | C |
| ATOM | 1558 | OD1 | ASP A | 242 | 30.621 | 5.232 | 32.747 | | | 1.00 | 88.58 | | A | O |
| ANISOU | 1558 | OD1 | ASP A | 242 | 11444 | 13166 | 9046 | −2919 | 433 | | | −2777 | A | O |
| ATOM | 1559 | OD2 | ASP A | 242 | 32.442 | 4.948 | 31.536 | | | 1.00 | 86.95 | | A | O1− |
| ANISOU | 1559 | OD2 | ASP A | 242 | 11091 | 13281 | 8667 | −2752 | 517 | | | −2831 | A | O1− |
| ATOM | 1560 | N | GLN A | 243 | 32.119 | 6.561 | 35.724 | | | 1.00 | 61.50 | | A | N |
| ANISOU | 1560 | N | GLN A | 243 | 8347 | 8781 | 6240 | −2478 | 238 | | | −2078 | A | N |
| ATOM | 1561 | CA | GLN A | 243 | 32.899 | 5.956 | 36.797 | | | 1.00 | 58.37 | | A | C |
| ANISOU | 1561 | CA | GLN A | 243 | 8213 | 7897 | 6066 | −2287 | 302 | | | −2122 | A | C |
| ATOM | 1562 | C | GLN A | 243 | 32.560 | 4.472 | 36.948 | | | 1.00 | 67.62 | | A | C |
| ANISOU | 1562 | C | GLN A | 243 | 9641 | 8795 | 7256 | −2343 | 499 | | | −2514 | A | C |
| ATOM | 1563 | O | GLN A | 243 | 31.439 | 4.051 | 36.630 | | | 1.00 | 66.00 | | A | O |
| ANISOU | 1563 | O | GLN A | 243 | 9430 | 8664 | 6982 | −2531 | 562 | | | −2688 | A | O |
| ATOM | 1564 | CB | GLN A | 243 | 32.650 | 6.668 | 38.136 | | | 1.00 | 49.50 | | A | C |
| ANISOU | 1564 | CB | GLN A | 243 | 7203 | 6431 | 5174 | −2204 | 182 | | | −1852 | A | C |
| ATOM | 1565 | CG | GLN A | 243 | 32.934 | 8.176 | 38.167 | | | 1.00 | 42.32 | | A | C |
| ANISOU | 1565 | CG | GLN A | 243 | 6062 | 5720 | 4296 | −2149 | −4 | | | −1456 | A | C |
| ATOM | 1566 | CD | GLN A | 243 | 34.427 | 8.513 | 38.178 | | | 1.00 | 49.20 | | A | C |
| ANISOU | 1566 | CD | GLN A | 243 | 6859 | 6603 | 5233 | −1932 | −26 | | | −1328 | A | C |
| ATOM | 1567 | OE1 | GLN A | 243 | 35.295 | 7.628 | 38.170 | | | 1.00 | 51.33 | | A | O |
| ANISOU | 1567 | OE1 | GLN A | 243 | 7245 | 6742 | 5515 | −1803 | 83 | | | −1530 | A | O |
| ATOM | 1568 | NE2 | GLN A | 243 | 34.728 | 9.807 | 38.190 | | | 1.00 | 43.34 | | A | N |
| ANISOU | 1568 | NE2 | GLN A | 243 | 5915 | 6017 | 4534 | −1892 | −159 | | | −1000 | A | N |
| ATOM | 1569 | N | PRO A | 244 | 33.512 | 3.650 | 37.422 | | | 1.00 | 70.28 | | A | N |
| ANISOU | 1569 | N | PRO A | 244 | 10189 | 8812 | 7701 | −2152 | 606 | | | −2641 | A | N |
| ATOM | 1570 | CA | PRO A | 244 | 33.291 | 2.206 | 37.623 | | | 1.00 | 75.79 | | A | C |
| ANISOU | 1570 | CA | PRO A | 244 | 11103 | 9214 | 8480 | −2098 | 794 | | | −2887 | A | C |
| ATOM | 1571 | C | PRO A | 244 | 32.114 | 1.882 | 38.541 | | | 1.00 | 74.33 | | A | C |
| ANISOU | 1571 | C | PRO A | 244 | 11039 | 8727 | 8477 | −2086 | 807 | | | −2776 | A | C |
| ATOM | 1572 | O | PRO A | 244 | 32.092 | 2.435 | 39.640 | | | 1.00 | 74.30 | | A | O |
| ANISOU | 1572 | O | PRO A | 244 | 11138 | 8467 | 8624 | −1979 | 712 | | | −2559 | A | O |
| ATOM | 1573 | CB | PRO A | 244 | 34.603 | 1.738 | 38.252 | | | 1.00 | 73.27 | | A | C |
| ANISOU | 1573 | CB | PRO A | 244 | 10976 | 8587 | 8277 | −1819 | 838 | | | −2904 | A | C |
| ATOM | 1574 | CG | PRO A | 244 | 35.623 | 2.703 | 37.720 | | | 1.00 | 68.87 | | A | C |
| ANISOU | 1574 | CG | PRO A | 244 | 10197 | 8350 | 7622 | −1766 | 721 | | | −2771 | A | C |
| ATOM | 1575 | CD | PRO A | 244 | 34.922 | 4.025 | 37.652 | | | 1.00 | 63.85 | | A | C |
| ANISOU | 1575 | CD | PRO A | 244 | 9337 | 7961 | 6960 | −1897 | 544 | | | −2470 | A | C |
| HETATM | 1576 | C1 | NAG A | 1076 | 47.849 | 6.531 | 51.063 | | | 1.00 | 44.10 | | A | C |
| HETATM | 1577 | C2 | NAG A | 1076 | 47.870 | 5.043 | 50.676 | | | 1.00 | 57.73 | | A | C |
| HETATM | 1578 | C3 | NAG A | 1076 | 48.738 | 4.258 | 51.649 | | | 1.00 | 62.74 | | A | C |
| HETATM | 1579 | C4 | NAG A | 1076 | 50.134 | 4.860 | 51.736 | | | 1.00 | 73.55 | | A | C |
| HETATM | 1580 | C5 | NAG A | 1076 | 50.074 | 6.367 | 52.006 | | | 1.00 | 66.00 | | A | C |
| HETATM | 1581 | C6 | NAG A | 1076 | 51.433 | 7.025 | 51.850 | | | 1.00 | 77.39 | | A | C |
| HETATM | 1582 | C7 | NAG A | 1076 | 45.835 | 4.269 | 49.503 | | | 1.00 | 69.45 | | A | C |
| HETATM | 1583 | CE | NAG A | 1076 | 44.486 | 3.638 | 49.680 | | | 1.00 | 73.06 | | A | C |
| HETATM | 1584 | N2 | NAG A | 1076 | 46.531 | 4.472 | 50.630 | | | 1.00 | 67.87 | | A | N |
| HETATM | 1585 | O3 | NAG A | 1076 | 48.812 | 2.907 | 51.207 | | | 1.00 | 61.48 | | A | O |
| HETATM | 1586 | O4 | NAG A | 1076 | 50.834 | 4.262 | 52.824 | | | 1.00 | 86.01 | | A | O |
| HETATM | 1587 | O5 | NAG A | 1076 | 49.177 | 7.032 | 51.099 | | | 1.00 | 44.53 | | A | O |
| HETATM | 1588 | O6 | NAG A | 1076 | 51.359 | 8.424 | 51.609 | | | 1.00 | 83.18 | | A | O |
| HETATM | 1589 | O7 | NAG A | 1076 | 46.264 | 4.592 | 48.392 | | | 1.00 | 59.22 | | A | O |
| HETATM | 1590 | C1 | NAG A | 2076 | 51.704 | 3.148 | 52.511 | | | 1.00 | 89.75 | | A | C |
| HETATM | 1591 | C2 | NAG A | 2076 | 52.777 | 3.076 | 53.594 | | | 1.00 | 84.61 | | A | C |
| HETATM | 1592 | C3 | NAG A | 2076 | 53.721 | 1.911 | 53.318 | | | 1.00 | 85.99 | | A | C |
| HETATM | 1593 | C4 | NAG A | 2076 | 52.918 | 0.618 | 53.266 | | | 1.00 | 85.51 | | A | C |
| HETATM | 1594 | C5 | NAG A | 2076 | 51.825 | 0.728 | 52.199 | | | 1.00 | 92.45 | | A | C |
| HETATM | 1595 | C6 | NAG A | 2076 | 50.898 | −0.470 | 52.172 | | | 1.00 | 89.30 | | A | C |
| HETATM | 1596 | C7 | NAG A | 2076 | 53.343 | 5.169 | 54.748 | | | 1.00 | 82.17 | | A | C |
| HETATM | 1597 | C8 | NAG A | 2076 | 54.144 | 6.438 | 54.698 | | | 1.00 | 79.48 | | A | C |
| HETATM | 1598 | N2 | NAG A | 2076 | 53.500 | 4.334 | 53.709 | | | 1.00 | 82.34 | | A | N |
| HETATM | 1599 | O3 | NAG A | 2076 | 54.714 | 1.836 | 54.336 | | | 1.00 | 95.43 | | A | O |
| HETATM | 1600 | O4 | NAG A | 2076 | 53.761 | −0.499 | 53.000 | | | 1.00 | 83.01 | | A | O |
| HETATM | 1601 | O5 | NAG A | 2076 | 50.999 | 1.883 | 52.440 | | | 1.00 | 95.99 | | A | O |
| HETATM | 1602 | O6 | NAG A | 2076 | 49.529 | −0.093 | 52.076 | | | 1.00 | 83.95 | | A | O |
| HETATM | 1603 | O7 | NAG A | 2076 | 52.591 | 4.908 | 55.693 | | | 1.00 | 78.70 | | A | O |
| HETATM | 1604 | C1 | NAG A | 1123 | 47.703 | 31.112 | 51.348 | | | 1.00 | 63.05 | | A | C |
| HETATM | 1605 | C2 | NAG A | 1123 | 48.304 | 32.500 | 51.518 | | | 1.00 | 72.32 | | A | C |
| HETATM | 1606 | C3 | NAG A | 1123 | 48.864 | 32.659 | 52.930 | | | 1.00 | 76.90 | | A | C |
| HETATM | 1607 | C4 | NAG A | 1123 | 47.778 | 32.420 | 53.977 | | | 1.00 | 85.17 | | A | C |
| HETATM | 1608 | C5 | NAG A | 1123 | 47.071 | 31.072 | 53.759 | | | 1.00 | 75.98 | | A | C |
| HETATM | 1609 | C6 | NAG A | 1123 | 45.790 | 30.947 | 54.564 | | | 1.00 | 66.09 | | A | C |
| HETATM | 1610 | C7 | NAG A | 1123 | 49.136 | 33.360 | 49.363 | | | 1.00 | 71.99 | | A | C |
| HETATM | 1611 | C8 | NAG A | 1123 | 50.348 | 33.540 | 48.494 | | | 1.00 | 68.30 | | A | C |
| HETATM | 1612 | N2 | NAG A | 1123 | 49.344 | 32.750 | 50.534 | | | 1.00 | 74.04 | | A | N |
| HETATM | 1613 | O3 | NAG A | 1123 | 49.408 | 33.969 | 53.060 | | | 1.00 | 73.86 | | A | O |
| HETATM | 1614 | O4 | NAG A | 1123 | 48.360 | 32.443 | 55.283 | | | 1.00 | 97.38 | | A | O |
| HETATM | 1615 | O5 | NAG A | 1123 | 46.695 | 30.884 | 52.381 | | | 1.00 | 77.46 | | A | O |
| HETATM | 1616 | O6 | NAG A | 1123 | 45.575 | 29.627 | 55.047 | | | 1.00 | 67.23 | | A | O |
| HETATM | 1617 | O7 | NAG A | 1123 | 48.021 | 33.741 | 49.014 | | | 1.00 | 73.84 | | A | O |
| HETATM | 1618 | C1 | NAG A | 2123 | 47.809 | 33.471 | 56.171 | | | 1.00 | 101.53 | | A | C |
| HETATM | 1619 | C2 | NAG A | 2123 | 48.615 | 33.558 | 57.507 | | | 1.00 | 98.63 | | A | C |
| HETATM | 1620 | C3 | NAG A | 2123 | 48.119 | 34.727 | 58.370 | | | 1.00 | 103.69 | | A | C |
| HETATM | 1621 | C4 | NAG A | 2123 | 48.083 | 36.025 | 57.574 | | | 1.00 | 109.93 | | A | C |
| HETATM | 1622 | C5 | NAG A | 2123 | 47.220 | 35.822 | 56.331 | | | 1.00 | 112.93 | | A | C |
| HETATM | 1623 | C6 | NAG A | 2123 | 47.087 | 37.055 | 55.460 | | | 1.00 | 112.42 | | A | C |
| HETATM | 1624 | C7 | NAG A | 2123 | 49.181 | 31.201 | 57.925 | | | 1.00 | 93.84 | | A | C |
| HETATM | 1625 | C8 | NAG A | 2123 | 48.955 | 30.016 | 58.818 | | | 1.00 | 90.46 | | A | C |
| HETATM | 1626 | N2 | NAG A | 2123 | 48.513 | 32.311 | 58.250 | | | 1.00 | 93.20 | | A | N |
| HETATM | 1627 | O3 | NAG A | 2123 | 48.935 | 34.886 | 59.527 | | | 1.00 | 100.98 | | A | O |
| HETATM | 1628 | O4 | NAG A | 2123 | 47.558 | 37.076 | 58.380 | | | 1.00 | 108.32 | | A | O |
| HETATM | 1629 | O5 | NAG A | 2123 | 47.794 | 34.783 | 55.522 | | | 1.00 | 111.35 | | A | O |
| HETATM | 1630 | O6 | NAG A | 2123 | 48.292 | 37.378 | 54.780 | | | 1.00 | 114.76 | | A | O |
| HETATM | 1631 | O7 | NAG A | 2123 | 49.932 | 31.153 | 56.951 | | | 1.00 | 98.48 | | A | O |
| HETATM | 1632 | C1 | NAG A | 1144 | 54.671 | 15.656 | 35.163 | | | 1.00 | 37.73 | | A | C |
| HETATM | 1633 | C2 | NAG A | 1144 | 54.274 | 14.196 | 35.065 | | | 1.00 | 37.73 | | A | C |
| HETATM | 1634 | C3 | NAG A | 1144 | 55.503 | 13.302 | 34.960 | | | 1.00 | 50.44 | | A | C |
| HETATM | 1635 | C4 | NAG A | 1144 | 56.531 | 13.777 | 33.938 | | | 1.00 | 51.62 | | A | C |
| HETATM | 1636 | C5 | NAG A | 1144 | 56.778 | 15.283 | 34.077 | | | 1.00 | 47.74 | | A | C |
| HETATM | 1637 | C6 | NAG A | 1144 | 57.543 | 15.877 | 32.914 | | | 1.00 | 55.32 | | A | C |
| HETATM | 1638 | C7 | NAG A | 1144 | 52.472 | 12.987 | 36.222 | | | 1.00 | 51.27 | | A | C |
| HETATM | 1639 | C8 | NAG A | 1144 | 51.855 | 12.689 | 37.554 | | | 1.00 | 50.70 | | A | C |
| HETATM | 1640 | N2 | NAG A | 1144 | 53.515 | 13.814 | 36.241 | | | 1.00 | 42.75 | | A | N |
| HETATM | 1641 | O3 | NAG A | 1144 | 55.056 | 11.989 | 34.646 | | | 1.00 | 60.19 | | A | O |
| HETATM | 1642 | O4 | NAG A | 1144 | 57.685 | 12.994 | 34.250 | | | 1.00 | 60.95 | | A | O |
| HETATM | 1643 | O5 | NAG A | 1144 | 55.528 | 15.984 | 34.100 | | | 1.00 | 47.14 | | A | O |
| HETATM | 1644 | O6 | NAG A | 1144 | 56.804 | 16.883 | 32.230 | | | 1.00 | 61.36 | | A | O |
| HETATM | 1645 | O7 | NAG A | 1144 | 52.016 | 12.538 | 35.177 | | | 1.00 | 59.72 | | A | O |
| HETATM | 1646 | C1 | NAG A | 2144 | 58.955 | 12.890 | 33.530 | | | 1.00 | 83.73 | | A | C |
| HETATM | 1647 | C2 | NAG A | 2144 | 59.539 | 11.488 | 33.820 | | | 1.00 | 94.96 | | A | C |
| HETATM | 1648 | C3 | NAG A | 2144 | 60.887 | 11.315 | 33.114 | | | 1.00 | 103.58 | | A | C |
| HETATM | 1649 | C4 | NAG A | 2144 | 60.782 | 11.691 | 31.639 | | | 1.00 | 103.06 | | A | C |
| HETATM | 1650 | C5 | NAG A | 2144 | 60.091 | 13.044 | 31.444 | | | 1.00 | 93.13 | | A | C |
| HETATM | 1651 | C6 | NAG A | 2144 | 59.775 | 13.317 | 29.996 | | | 1.00 | 88.48 | | A | C |
| HETATM | 1652 | C7 | NAG A | 2144 | 58.862 | 10.457 | 35.937 | | | 1.00 | 89.36 | | A | C |
| HETATM | 1653 | C8 | NAG A | 2144 | 59.149 | 10.326 | 37.405 | | | 1.00 | 83.94 | | A | C |
| HETATM | 1654 | N2 | NAG A | 2144 | 59.684 | 11.256 | 35.247 | | | 1.00 | 91.04 | | A | N |
| HETATM | 1655 | O3 | NAG A | 2144 | 61.315 | 9.960 | 33.242 | | | 1.00 | 102.99 | | A | O |
| HETATM | 1656 | O4 | NAG A | 2144 | 62.078 | 11.733 | 31.048 | | | 1.00 | 106.07 | | A | O |
| HETATM | 1657 | O5 | NAG A | 2144 | 58.829 | 13.066 | 32.129 | | | 1.00 | 93.17 | | A | O |
| HETATM | 1658 | O6 | NAG A | 2144 | 59.024 | 12.239 | 29.453 | | | 1.00 | 86.36 | | A | O |
| HETATM | 1659 | O7 | NAG A | 2144 | 57.921 | 9.870 | 35.399 | | | 1.00 | 93.33 | | A | O |
| HETATM | 1660 | C1 | NAG A | 1228 | 54.131 | 12.661 | 30.331 | | | 1.00 | 73.34 | | A | C |
| HETATM | 1661 | C2 | NAG A | 1228 | 55.401 | 12.376 | 29.460 | | | 1.00 | 83.15 | | A | C |
| HETATM | 1662 | C3 | NAG A | 1228 | 55.578 | 10.871 | 29.213 | | | 1.00 | 92.37 | | A | C |
| HETATM | 1663 | C4 | NAG A | 1228 | 55.493 | 10.061 | 30.498 | | | 1.00 | 93.75 | | A | C |
| HETATM | 1664 | C5 | NAG A | 1228 | 54.194 | 10.379 | 31.232 | | | 1.00 | 90.93 | | A | C |
| HETATM | 1665 | C6 | NAG A | 1228 | 54.052 | 9.631 | 32.538 | | | 1.00 | 89.70 | | A | C |
| HETATM | 1666 | C7 | NAG A | 1228 | 55.953 | 14.259 | 27.962 | | | 1.00 | 62.50 | | A | C |
| HETATM | 1667 | C8 | NAG A | 1228 | 55.801 | 14.820 | 26.580 | | | 1.00 | 61.44 | | A | C |
| HETATM | 1668 | N2 | NAG A | 1228 | 55.354 | 13.082 | 28.186 | | | 1.00 | 77.40 | | A | N |
| HETATM | 1669 | O3 | NAG A | 1228 | 56.846 | 10.634 | 28.609 | | | 1.00 | 97.10 | | A | O |
| HETATM | 1670 | O4 | NAG A | 1228 | 55.536 | 8.679 | 30.152 | | | 1.00 | 98.74 | | A | O |
| HETATM | 1671 | O5 | NAG A | 1228 | 54.141 | 11.786 | 31.533 | | | 1.00 | 87.29 | | A | O |
| HETATM | 1672 | O6 | NAG A | 1228 | 55.154 | 9.888 | 33.397 | | | 1.00 | 97.52 | | A | O |
| HETATM | 1673 | O7 | NAG A | 1228 | 56.570 | 14.855 | 28.842 | | | 1.00 | 51.25 | | A | O |
| HETATM | 1674 | C1 | NAG A | 2228 | 56.712 | 7.979 | 30.648 | | | 1.00 | 105.75 | | A | C |
| HETATM | 1675 | C2 | NAG A | 2228 | 56.432 | 6.506 | 30.363 | | | 1.00 | 109.04 | | A | C |
| HETATM | 1676 | C3 | NAG A | 2228 | 57.551 | 5.638 | 30.941 | | | 1.00 | 114.26 | | A | C |
| HETATM | 1677 | C4 | NAG A | 2228 | 58.904 | 6.090 | 30.393 | | | 1.00 | 114.48 | | A | C |
| HETATM | 1678 | C5 | NAG A | 2228 | 59.110 | 7.592 | 30.626 | | | 1.00 | 116.17 | | A | C |
| HETATM | 1679 | C6 | NAG A | 2228 | 60.393 | 8.132 | 30.026 | | | 1.00 | 116.84 | | A | C |
| HETATM | 1680 | C7 | NAG A | 2228 | 54.072 | 5.897 | 30.099 | | | 1.00 | 103.32 | | A | C |
| HETATM | 1681 | C8 | NAG A | 2228 | 54.306 | 6.058 | 28.626 | | | 1.00 | 104.80 | | A | C |
| HETATM | 1682 | N2 | NAG A | 2228 | 55.133 | 6.111 | 30.886 | | | 1.00 | 105.10 | | A | N |
| HETATM | 1683 | O3 | NAG A | 2228 | 57.316 | 4.272 | 30.614 | | | 1.00 | 114.03 | | A | O |
| HETATM | 1684 | O4 | NAG A | 2228 | 59.957 | 5.358 | 31.014 | | | 1.00 | 109.16 | | A | O |
| HETATM | 1685 | O5 | NAG A | 2228 | 58.018 | 8.350 | 30.070 | | | 1.00 | 113.09 | | A | O |
| HETATM | 1686 | O6 | NAG A | 2228 | 60.410 | 8.042 | 28.608 | | | 1.00 | 118.16 | | A | O |
| HETATM | 1687 | O7 | NAG A | 2228 | 52.975 | 5.578 | 30.552 | | | 1.00 | 99.40 | | A | O |
| TER | 1688 | | NAG A | 2228 | | | | | | | | | | |
| HETATM | 1688 | O | HOH S | 1 | 31.421 | 19.873 | 44.880 | | | 1.00 | 42.21 | | S | O |
| HETATM | 1689 | O | HOH S | 3 | 41.885 | 20.609 | 55.460 | | | 1.00 | 40.39 | | S | O |
| HETATM | 1690 | O | HOH S | 4 | 60.490 | 20.808 | 51.222 | | | 1.00 | 39.49 | | S | O |
| HETATM | 1691 | | HOH S | 5 | 29.548 | 12.649 | 29.611 | | | 1.00 | 24.15 | | S | O |
| TER | 1693 | | HOH S | 5 | | | | | | | | | | |
| END | | | | | | | | | | | | | | |

What is claimed is:

1. A plant, plant part, plant cell, or plant seed comprising a modified plant LysM receptor, wherein the modified plant LysM receptor comprises a modified LysM domain with an altered affinity, selectivity, specificity, or combination thereof for an oligosaccharide or class of oligosaccharides as compared to the unmodified plant LysM receptor, wherein the modified plant LysM receptor was generated by:
(a) providing a structural model, a molecular model, a surface characteristics model, an electrostatic potential model, or combinations thereof of a donor plant LysM receptor having the desired receptor characteristic and two or more potential target plant LysM receptors, wherein the desired receptor characteristic is affinity, selectivity, specificity, or combinations thereof for the oligosaccharide or class of oligosaccharides;
(b) comparing each of the two or more potential target plant LysM receptors with the structural model, the molecular model, the surface characteristics model, the electrostatic potential model of the donor plant LysM receptor, or combinations thereof; or comparing each of the two or more potential target plant LysM receptors with the donor plant LysM receptor using structural overlay;
(c) selecting the potential target plant LysM receptor for modification;
(d) identifying one or more amino acid residues for modification in the target LysM receptor by comparing amino acid residues of a first oligosaccharide binding feature in the donor plant LysM receptor with the corresponding amino acid residues in the target plant LysM receptor; and
(e) generating a modified plant LysM receptor wherein the one or more amino acid residues in the first oligosaccharide binding feature of the target plant LysM receptor have been substituted with corresponding amino acid residues from the donor plant LysM receptor.

2. The plant, plant part, plant cell, or plant seed of claim 1, wherein the structural modeling was performed using SWISS-MODEL, PDB2PQR, APBS, PyMol, and APBS tools 2.1.

3. The plant, plant part, plant cell, or plant seed of claim 1, wherein the class of oligosaccharides is a lipo-chitooligosaccharide (LCO).

4. The plant, plant part, plant cell, or plant seed of claim 3, wherein the modified LysM domain binds the LCO with higher affinity and/or selectivity than the unmodified LysM domain.

5. The plant, plant part, plant cell, or plant seed of claim 4, wherein the higher affinity and/or selectivity is due to a hydrophobic patch interacting with the LCO or with the lipid of the LCO.

6. The plant, plant part, plant cell, or plant seed of claim 3, wherein the LCO is produced by nitrogen-fixing bacteria or by mycorrhizal fungi.

7. The plant, plant part, plant cell, or plant seed of claim 6, wherein the LCO is produced by nitrogen-fixing bacteria selected from the group consisting of Mesorhizobium loti, Mesorhizobium huakuii, Mesorhizobium mediterraneum, Mesorhizobium ciceri, Mesorhizobium spp., Rhizobium mongolense, Rhizobium tropici, Rhizobium etli phaseoli, Rhizobium giardinii, Rhizobium leguminosarum optionally R. leguminosarum trifolii, R. leguminosarum viciae, and R. leguminosarum phaseoli, Burkholderiales optionally symbionts of Mimosa, Sinorhizobium meliloti, Sinorhizobium medicae, Sinorhizobium fredii, Sinorhizobium NGR234, Azorhizobium caulinodans, Bradyrhizobium japonicum, Bradyrhizobium elkanii, Bradyrhizobium liaonginense, Frankia spp., and any combination thereof, or by or by mycorrhizal fungi selected from the group consisting of Acaulosporaceae spp., Diversisporaceae spp., Gigasporaceae spp., Pacisporaceae spp., Funneliformis spp., Glomus spp., Rhizophagus spp., Sclerocystis spp., Septoglomus spp., Claroideoglomus spp., Ambispora spp., Archaeospora spp., Geosiphon pyriformis, Paraglomus spp., other species in the division Glomeromycota, and any combination thereof.

8. The plant, plant part, plant cell, or plant seed of claim 1, wherein the LysM receptor is selected from the group consisting of a LysM chitooligosaccharide (CO) receptor, a LysM LCO receptor, and a LysM peptidoglycan (PGN) receptor.

9. The plant, plant part, plant cell, or plant seed of claim 8, wherein the LysM receptor is a LysM CO receptor or a LysM LCO receptor comprising a hydrophobic patch, and wherein the hydrophobic patch is adjacent to a chitin binding motif.

10. The plant, plant part, plant cell, or plant seed of claim 8, wherein the LysM receptor is a LysM CO receptor or a LysM LCO receptor comprising a hydrophobic patch, wherein the hydrophobic patch is within 30 Å, 20 Å, 10 Å, 7.5 Å, 5 Å, 4 Å, 3 Å, 2 Å, 1.5 Å, or 1 Å of a chitin binding motif.

11. The plant, plant part, plant cell, or plant seed of claim 8, wherein the LysM receptor is a LysM PGN receptor comprising a hydrophobic patch, and wherein the hydrophobic patch is adjacent to a glycan binding motif.

12. The plant, plant part, plant cell, or plant seed of claim 8, wherein the LysM receptor is a LysM PGN receptor comprising a hydrophobic patch, and wherein the hydrophobic patch is within 30 Å, 20 Å, 10 Å, 7.5 Å, 5 Å, 4 Å, 3 Å, 2 Å, 1.5 Å, or 1 Å of a glycan binding motif.

13. The plant, plant part, plant cell, or plant seed of claim 1, wherein the LysM receptor is not an exopolysaccharide (EPS) receptor.

14. The plant, plant part, plant cell, or plant seed of claim 1, wherein the LysM receptor is a polypeptide having at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to SEQ ID NO:34 (i.e., Lotus CERK6; BAI79273.1_LJCERK6), or wherein the LysM receptor is a polypeptide having amino acid sequence SEQ ID NO:34 (i.e., Lotus CERK6; BAI79273.1_LjCERK6).

15. The plant, plant part, plant cell, or plant seed of claim 1, wherein the modified LysM receptor comprises a LysM2 domain modified to comprise a hydrophobic patch on the surface of the LysM2 domain.

16. The plant, plant part, plant cell, or plant seed of claim 15, wherein the one or more amino acid residues in the first oligosaccharide:
(i) include amino acids corresponding to SEQ ID NO: 63 and SEQ ID NO: 64 of Lotus japonicus NFR5,
(ii) include amino acids corresponding to L147, L151, L152, L154, T156, K157, and V158 of Medicago NFP (SEQ ID NO: 1), or
(iii) the one or more amino acid residues in the first oligosaccharide binding feature include combinations of (i) and (ii).

17. The plant, plant part, plant cell, or plant seed of claim 16, wherein the one or more amino acid residues in the first oligosaccharide binding feature include amino acids corresponding to SEQ ID NO: 63 and SEQ ID NO: 64 of Lotus japonicus NFR5.

18. The plant, plant part, plant cell, or plant seed of claim 16, wherein the one or more amino acid residues in the first oligosaccharide binding feature forming the hydrophobic patch include amino acids corresponding to L147, L151, L152, L154, T156, K157, and V158 of Medicago NFP (SEQ ID NO: 1).

19. The plant, plant part, plant cell, or plant seed of claim 15, wherein the hydrophobic patch was generated by deleting at least one non-hydrophobic amino acid residue, substituting at least one amino acid residue with a more hydrophobic amino acid, or combinations thereof.

20. The plant, plant part, plant cell, or plant seed of claim 15, wherein the hydrophobic patch was generated by modifying an existing hydrophobic patch in the unmodified LysM receptor.

21. The plant, plant part, plant cell, or plant seed of claim 20, wherein the unmodified LysM receptor was modified by deleting at least one non-hydrophobic amino acid residue, substituting at least one amino acid residue with a more hydrophobic amino acid, substituting at least one hydrophobic amino acid residue with another hydrophobic amino acid residue, or combinations thereof.

22. The plant, plant part, plant cell, or plant seed of claim 1, wherein the modified plant LysM receptor comprises a first LysM1 domain modified to replace at least part of the first LysM1 domain with at least part of a second LysM1 domain.

23. The plant, plant part, plant cell, or plant seed of claim 22, wherein the one or more amino acid residues in the first oligosaccharide include amino acids corresponding to SEQ ID NO: 28 and SEQ ID NO: 29 of Lotus NFR1 and/or SEQ ID NO: 30 and SEQ ID NO: 31 of Lotus CERK6.

24. The plant, plant part, plant cell, or plant seed of claim 22, wherein the first LysM1 domain is modified by substituting a first part of the first LysM1 domain with a third part of a second LysM1 domain and/or by substituting a second part of the first LysM1 domain with a fourth part of the second LysM1 domain.

25. The plant, plant part, plant cell, or plant seed of claim 22, wherein the first LysM1 domain and the second LysM1 domain have different affinities, selectivities, and/or specificities for oligosaccharides, and the modification of the first LysM1 domain alters the affinity, selectivity, and/or specificity of the first LysM1 domain to be more like the second LysM1 domain.

26. The plant, plant part, plant cell, or plant seed of claim 24, wherein the first part and the third part correspond to SEQ ID NO:30 [Lotus CERK6 region II 43-53] or NGSNLTYISEI, SEQ ID NO: 28 [Lotus NFR1 region II 41-52] or PGVFILQNITTF; and wherein the second part and the fourth part correspond to SEQ ID NO:31 [Lotus CERK6 region IV 74-82] or ASKDSVQAG; SEQ ID NO:29 [Lotus NFR1 region IV 73-81], or LNDINIQSF.

27. The plant, plant part, plant cell, or plant seed of claim 24, wherein the first LysM1 domain is selected from the group consisting of SEQ ID NO:32 [LysM1 domain Lotus NFR1; LjNFR1/26-95], SEQ ID NO:33 [LysM1 domain Medicago LYK3; MtLYK3/25-95], and NFR1 DLALASYYILPGVFILQNITTFMQSEIVSSNDAITSYNKD KILNDINIQSFORLNIPFP (SEQ ID NO: 106); and the second LysM1 domain is CERK6:
(SEQ ID NO: 107)
ALAQASYYLLNGSNLTYISEIMQSSLLTKPEDIVSYNQDTIASKDSV
QAGQRINVPFP.

28. The plant, plant part, plant cell, or plant seed of claim 27, wherein
the first part is selected from SEQ ID NO:30 [Lotus CERK6 region II 43-53] and NGSNLTYISEI;
wherein the second part is selected from SEQ ID NO:31 [Lotus CERK6 region IV 74-82] and ASKDSVQAG;
wherein the third part is selected from SEQ ID NO:28 [Lotus NFR 1 region II 41-52] and PGVFILQNITTF; and
wherein the fourth part is selected from SEQ ID NO:29 [Lotus NFR1 region IV 73-81] and LNDINIQSF.

29. The plant, plant part, plant cell, or plant seed of claim 24, wherein the first LysM1 domain is further modified by substituting a fifth part of the first LysM1 domain with a sixth part of a second LysM1 domain.

30. The plant, plant part, plant cell, or plant seed of claim 29, wherein the first LysM1 domain is SEQ ID NO:32 [LysM1 domain Lotus NFR1; LjNFR1/26-95] and the second LysM1 domain is SEQ ID NO:33 [LysM1 domain Medicago LYK3; MtLYK3/25-95].

31. The plant, plant part, plant cell, or plant seed of claim 29, wherein the fifth part is SEQ ID NO: 53 [Lotus NFR1 region III 59-62; LjNFR1/56-92], and wherein the sixth part is SEQ ID NO: 46 [Medicago LYK3 region III 57-62; MtLYK3/57-62].

32. The plant, plant part, plant cell, or plant seed of claim 29, wherein the first LysM1 domain is modified by substituting a seventh part of the first LysM1 domain, wherein the seventh part spans the first part of the first LysM1 domain, the second part of the first LysM1 domain, and the fifth part of the first LysM1 domain, with an eighth part of the second LysM1 domain, wherein the eighth part spans the third part of the second LysM1 domain, the fourth part of the second LysM1 domain, and the sixth part of the second LysM1 domain.

33. The plant, plant part, plant cell, or plant seed of claim 32, wherein the seventh part of the first LysM1 domain is SEQ ID NO: 51 [Lotus NFR1 regions II-IV 41-82; LjNFR1/41-82], and wherein the eighth part of the second LysM1 domain is SEQ ID NO: 104 [Medicago LYK3 regions II-IV 41-82; MtLYK3/41-82].

34. The plant, plant part, plant cell, or plant seed of claim 29, wherein the first LysM1 domain is SEQ ID NO:33 [LysM1 domain Medicago LYK3; MtLYK3/25-95] and the second LysM1 domain is SEQ ID NO:32 [LysM1 domain Lotus NFR1; LjNFR1/26-95].

35. The plant, plant part, plant cell, or plant seed of claim 34, wherein the fifth part is SEQ ID NO: 46 [Medicago LYK3 region III 57-62; MtLYK3/57-62], and wherein the sixth part is SEQ ID NO: 53 [Lotus NFR 1 region III 59-62; LjNFR1/56-92].

36. The plant, plant part, plant cell, or plant seed of claim 34, wherein the first LysM1 domain is modified by substituting a seventh part of the first LysM1 domain, wherein the seventh part spans the first part of the first LysM1 domain, the second part of the first LysM1 domain, and the fifth part of the first LysM1 domain, with an eighth part of the second LysM1 domain, wherein the eighth part spans the third part of the second LysM1 domain, the fourth part of the second LysM1 domain, and the sixth part of the second LysM1 domain.

37. The plant, plant part, plant cell, or plant seed of claim 36, wherein the seventh part of the first LysM1 domain is SEQ ID NO: 104 [Medicago LYK3 regions II-IV 41-82; MtLYK3/41-82], and wherein the eighth part of the second LysM1 domain is SEQ ID NO: 51 [Lotus NFR1 regions II-IV 41-82; LjNFR1/41-82].

38. The plant, plant part, plant cell, or plant seed of claim 22, wherein the entire first LysM1 domain was replaced with the entire second LysM1 domain.

39. The plant, plant part, plant cell, or plant seed of claim 22, wherein 80% or fewer, 70% or fewer, 60% or fewer, 50% or fewer, 40% or fewer, 30% or fewer, or 20% or fewer of amino acid residues in the first LysM1 domain were substituted or deleted with the corresponding amino acid residues of the second LysM1 domain and the entire LysM1 domain in the unmodified plant LysM receptor was not substituted with another entire LysM2 domain to generate the modified plant LysM receptor.

40. A plant, plant part, plant cell, or plant seed comprising a modified plant LysM receptor, wherein the modified plant LysM receptor comprises a LysM2 domain modified to comprise a hydrophobic patch on the surface of the LysM2 domain, wherein one or more amino acid residues in a first oligosaccharide binding feature of the plant LysM receptor have been substituted with corresponding amino acid residues from a donor plant LysM receptor; wherein
(i) the one or more amino acid residues in the first oligosaccharide binding feature include amino acids corresponding to SEQ ID NO: 63 and SEQ ID NO: 64 of Lotus japonicus NFR5,
(ii) the one or more amino acid residues in the first oligosaccharide binding feature include amino acids corresponding to L147, L151, L152, L154, T156, K157, and V158 of Medicago NFP (SEQ ID NO: 1), or
(iii) the one or more amino acid residues in the first oligosaccharide binding feature include combinations of (i) and (ii).

41. A plant, plant part, plant cell, or plant seed comprising a modified plant LysM receptor, wherein the modified plant LysM receptor comprises a first LysM1 domain modified to replace at least part of the first LysM1 domain with at least part of a second LysM1 domain, wherein one or more amino acid residues in a first oligosaccharide binding feature of the plant LysM receptor have been substituted with corresponding amino acid residues from a donor plant LysM receptor; wherein the one or more amino acid residues in the first oligosaccharide include amino acids corresponding to SEQ ID NO: 28 and SEQ ID NO: 29 of Lotus NFR1 and/or SEQ ID NO: 30 and SEQ ID NO: 31 of Lotus CERK6.
